SEQUENCE LISTING (31 sequences)

SEQ ID NO: 1 - protein, 585 aa, Homo sapiens [sequence omitted]

SEQ ID NO: 2 - protein, 593 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGHHHHHH

SEQ ID NO: 3 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 484 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 5 aa, synthetic construct
GGGGS

SEQ ID NO: 6 - protein, 10 aa, synthetic construct
GGGGSGGGGS

SEQ ID NO: 7 - protein, 15 aa, synthetic construct
GGGGSGGGGSGGGGS

SEQ ID NO: 8 - protein, 20 aa, synthetic construct
GGGGSGGGGSGGGGSGGGGS

SEQ ID NO: 9 - protein, 25 aa, synthetic construct
GGGGSGGGGSGGGGSGGGGSGGGGS

SEQ ID NO: 10 - protein, 93 aa, synthetic construct
PVITTPLETVDALVEEVATFMCAVESYPQPEISWTRNKILIKLFDTRYSIRENGQLLTILSVEDSDDGIYCCTANNGVGGAVESCGALQVKMK

SEQ ID NO: 11 - protein, 188 aa, synthetic construct
EKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVE

SEQ ID NO: 12 - protein, 221 aa, synthetic construct
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVNWTCATRLFPDFFTRVALYVDWIRSTLR

SEQ ID NO: 13 - protein, 211 aa, synthetic construct
SEHETRLEAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVNFTKVLLQYTGHITWTPPAIFKSYCEIIVTHFPFDEQNCSMKLGTRTYDGSAVAINPESDQPDLSNFMESGEWVIKESRGWKHSVTYSSSPDTPYLDITYHFVMQRLP

SEQ ID NO: 14 - protein, 73 aa, synthetic construct
EEVRKLKARVDELERIRRSILPYGDSMDRIEKDRLQGMAPAAGADLDKIGLHSDSQEELWMFVRKKLMMEQEN

SEQ ID NO: 15 - protein, 239 aa, synthetic construct
MGCSSPPCECHQEEDFRVTCKDIQRIPSLPPSTQTLKLIETCLRTIPSHAFSNLPNISRIYVSIDVTLQQLESHSFYNLSKVTHIEIRNTPNLTYIDPDALKELPLLKFLGIFNTGLKMFPPLTKVYSTEIFFILEITDNPYMTSIPRNAFQGLCNETLTLKLYNNGFTSVQGYAFNGTKLDAVYLNKNKYLTVIDKDAFGGVYSGPSLLDVSQTSVTALPSKGLEHLKELRARNTWTL

SEQ ID NO: 16 - protein, 339 aa, synthetic construct
PVITTPLETVDALVEEVATFMCAVESYPQPEISWTRNKILIKLFDTRYSIRENGQLLTILSVEDSDDGIYCCTANNGVGGAVESCGALQVKMKIEGRGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 434 aa, synthetic construct
EKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEIEGRGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 788 aa, synthetic construct
EKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - protein, 829 aa, synthetic construct
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDAGGPLICDGIIQGIDSFVNWTCATRLFPDFFTRVALYVDWIRSTLRGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGHHHHHH

SEQ ID NO: 20 - protein, 811 aa, synthetic construct
SEHETRLEAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVNFTKVLLQYTGHITWTPPAIFKSYCEIIVTHFPFDEQNCTMKLGTRTYDGSAVAINPESDQPDLSNFMESGEWVIKESRGWKHSVTYSSSPDTPYLDITYHFVMQRLPGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 21 - protein, 563 aa, synthetic construct
MDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEEVRKLKARVDELERIRRSILPYGDSMDRIEKDRLQGMAPAAGADLDKIGLHSDSQEELWMFVRKKLMMEQEN

SEQ ID NO: 22 - protein, 480 aa, synthetic construct
GCSSPPCECHQEEDFRVTCKDIQRIPSLPPSTQTLKLIETCLRTIPSHAFSNLPNISRIYVSIDVTLQQLESHSFYNLSKVTHIEIRNTPNLTYIDPDALKELPLLKFLGIFNTGLKMFPPLTKVYSTEIFFILEITDNPYMTSIPRNAFQGLCNETLTLKLYNNGFTSVQGYAFNGTKLDAVYLNKNKYLTVIDKDAFGGVYSGPSLLDVSQTSVTALPSKGLEHLKELRARNTWTLGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 851 aa, synthetic construct
MGCSSPPCECHQEEDFRVTCKDIQRIPSLPPSTQTLKLIETCLRTIPSHAFSNLPNISRIYVSIDVTLQQLESHSFYNLSKVTHIEIRNTPNLTYIDPDALKELPLLKFLGIFNTGLKMFPPLTKVYSTEIFFILEITDNPYMTSIPRNAFQGLCNETLTLKLYNNGFTSVQGYAFNGTKLDAVYLNKNKYLTVIDKDAFGGVYSGPSLLDVSQTSVTALPSKGLEHLKELRARNTWTLIEGRGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGHHHHHH

SEQ ID NO: 24 - protein, 609 aa, Homo sapiens [sequence omitted]

SEQ ID NO: 25 - protein, 585 aa, Homo sapiens [sequence omitted]

SEQ ID NO: 26 - protein, 205 aa, Homo sapiens [sequence omitted]

SEQ ID NO: 27 - protein, 399 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 28 - protein, 410 aa, synthetic construct
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 29 - protein, 614 aa, synthetic construct
EEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 30 - protein, 604 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 31 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0224719 A1
(43) Pub. Date: Aug. 6, 2026

(54) LYSOSOMAL TARGETING BIFUNCTIONAL MOLECULES FOR DEGRADATION OF AUTOANTIBODIES

(71) Applicant: Lycia Therapeutics, Inc., South San Francisco, CA (US)

(72) Inventors: Darrin Anthony Lindhout, Mountain View, CA (US); Tao Chen, Palo Alto, CA (US); Richard Glynne, South San Francisco, CA (US); Jeffrey Iwig, South San Francisco, CA (US); Jason G. Lewis, Castro Valley, CA (US); Diana Li, Daly City, CA (US); Nicholas Lind, Lafayette, CA (US); Matthew Shurtleff, Redwood City, CA (US); Steven Staben, Emerald Hills, CA (US); Sarah Michelle Totten, Danville, CA (US); Eric D. Turtle, Belmont, CA (US)

(21) Appl. No.: 19/148,946

(22) PCT Filed: Jan. 17, 2024
(86) PCT No.: PCT/US2024/011901

§ 371 Date: Jul. 17, 2025

Related U.S. Application Data
(60) Provisional application No. 63/480,509, filed on Jan. 18, 2023.
(60) Provisional application No. 63/518,537, filed on Aug. 9, 2023.

(51) Int. Cl. A61K 47/66; A61K 47/54; A61K 47/64; A61K 47/68

(52) U.S. Cl. CPC A61K 47/66; A61K 47/549; A61K 47/6425; A61K 47/643; A61K 47/6889; C07K 2319/30

(57) ABSTRACT

The present disclosure provides bifunctional molecules including a moiety that targets a lysosomal targeting molecule, a linker, and a polypeptide that specifically binds a extracellular target molecule such as an antibody that is targeted for degradation.

### CROSS-REFERENCE TO RELATED APPLICATIONS

This application claims the benefit under 35 U.S.C. 119(e) of U.S. Provisional Application No. 63/480,509, filed Jan. 18, 2023 and 63/518,537, filed on Aug. 9, 2023, each of which are incorporated by reference in their entirety.

### BACKGROUND

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches.

Autoimmune disease occurs when the body's immune response mistakenly targets an individual's tissue, eventually leading to organ destruction or dysfunction. Autoantibodies recognize autoantigens and are often associated with autoimmune diseases. Conventional treatments for autoimmune diseases can control the overactive immune response and reduce inflammation or pain but often cause undesirable side effects.

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches. Thus, there is a need for therapies that can target a wider range of proteins.

Lysosome-targeting chimeras (LYTACs) are bifunctional molecules that provide for selective protein degradation via binding to cell surface receptors. The first LYTACs targeted extracellular proteins for degradation via engaging the cation-independent mannose-6-phosphate receptor (CI-M6PR) or the asialoglycoprotein receptor (ASGPR). ASGPR is the transmembrane glycoprotein receptor found primarily in hepatocytes which plays an important role in serum glycoprotein homeostasis by mediating the endocytosis and lysosomal degradation of glycoproteins with exposed terminal galactose or N-acetylgalactosamine (GalNAc) residues. ASGPR cycles between endosomes and the cell surface.

### SUMMARY

The present disclosure provides a class of lysosomal targeting bifunctional molecules that include a ligand moiety that specifically binds to a lysosomal targeting receptor on the surface of a cell (also referred to as a cell internalizing receptor), a linker and a polypeptide that specifically binds to an extracellular target molecule.

The inventors have demonstrated that bifunctional molecules (also referred to as conjugates) of this disclosure having a particular configuration of ligand binding moieties (X) with a linker of desired valency and/or length can specifically bind with high affinity to both the internalizing receptor and an extracellular target molecule such as an autoantibody simultaneously and exhibit high uptake activity of the target molecule. The conjugates of this disclosure can provide for sequestering and degrading of a target molecule in the cell's lysosome.

The lysosomal targeting bifunctional molecules bind extracellular target molecules via the target-binding polypeptide and bind cell internalizing receptor via the ligand moiety. Binding of the ligand moiety to cell internalizing receptor can trigger internalization of the bifunctional molecule and bound target molecule. The bifunctional molecule described herein can then facilitate transport of the into a cell and can facilitate sequestration and/or degradation of the target autoantibody in the cell's lysosome. In some embodiments, the extracellular target molecule is an autoantibody, and the polypeptide of the bifunctional molecule is a protein antigen of the autoantibody.

Also provided herein are compositions comprising such bifunctional molecules and methods of using the bifunctional molecules to target an extracellular target molecule of interest for sequestration and/or lysosomal degradation.

### BRIEF DESCRIPTION OF THE DRAWINGS

FIG. 1 illustrates an exemplary mechanism of action of an exemplary bifunctional molecule (e.g., target binding conjugate) to bind a lysosomal targeting receptor on the surface of a cell (also referred to as a cell internalizing receptor), to facilitate transport into the cell and facilitate sequestration and/or degradation of the target molecule (e.g., autoantibody) in the cell's lysosome.

FIGS. 2A-2D shows a schematic illustration of various displays of immunogenic domains capable of binding to antibodies. The domains can be designed and expressed as genetic fusions containing N/C-terminal tags including human Fc (FIGS. 2A & 2B), human serum albumin (FIG. 2C), and as a human serum albumin containing a 6×-poly-histidine (FIG. 2D) as described herein according to various embodiments of the present disclosure.

FIGS. 3A-3B illustrate in vitro uptake of patient derived autoantibodies in human hepatocellular carcinoma (HepG2) cells using target binding conjugates described herein.

FIGS. 4A-4D illustrate selective autoantibody uptake with an exemplary target binding conjugate.

FIGS. 5A-C show the ability of MuSK conjugates to mediate lysosomal delivery of patient-derived pathogenic anti-MuSK antibodies.

FIGS. 6A-6B show the ability of conjugates to clear the patient-derived antibody 13-3B5 from mouse serum.

FIGS. 7A-B illustrate in vitro uptake of patient derived autoantibodies in human hepatocellular carcinoma (HepG2) cells using thermostable TSHR variant-based target binding conjugates with Fe carrier polypeptide as described herein.

FIGS. 8A-8B illustrate uptake of patient-derived autoantibodies using thermostable TSHR variant-based target binding conjugates fused to HSA as described herein in human HepG2 cells.

FIGS. 9A-9B show AChR conjugate-mediated cellular uptake of the patient-derived antibody MAB637 and the pathogenic tool antibody MAB35.

FIG. 10 illustrates the ability of an AChR conjugate to induce degradation of pathogenic antibodies in Hep G2 cells.

FIGS. 11A-11B show BP180 conjugate-mediated cellular uptake of the patient-derived antibody 3-30G.

FIG. 12 illustrates the ability of a BP180 conjugate to induce degradation of the pathogenic antibody 3-30G in Hep G2 cells.

FIGS. 13A-13B show PR3 conjugate-mediated cellular uptake of the pathogenic antibody 4A5.

FIG. 14 illustrates the ability of a PR3 conjugate to induce degradation of the pathogenic antibody 4A5 in Hep G2 cells.

### DETAILED DESCRIPTION

### Lysosomal Targeting Bifunctional Molecules

As summarized above, this disclosure provides lysosomal targeting bifunctional molecules (also referred to as LYTACs) that target disease causing extracellular target molecules, e.g., autoantibodies, for degradation. The lysosomal targeting bifunctional molecules include a ligand moiety that specifically binds to a lysosomal targeting receptor (i.e., an internalizing receptor), and which is linked to a bait polypeptide that specifically binds target molecules, e.g., autoantibodies. In certain embodiments, the lysosomal targeting receptor is a lysosomal cell surface receptor (e.g., asialoglycoprotein receptor [ASGPR], or cation-independent mannose-6-phosphate receptor [CI-M6PR]).

In some embodiments, a LYTAC binds pathogenic autoantibodies in circulation and forms a ternary complex with the cell internalizing receptor, e.g., a liver-specific internalizing receptor, ASGPR. After clathrin-mediated endocytosis, the protein complex progresses through the endocytic pathway whereby the cell internalizing receptor (e.g., ASGPR) dissociates due to decreasing pH and Ca²⁺ levels and is recycled to the cell surface. A LYTAC and the pathogenic antibody continue to the lysosome where they are degraded by lysosomal proteases.

The inventors have demonstrated that bifunctional molecules (e.g., conjugates) of this disclosure having a particular configuration of ligand binding moieties (X) with a linker of desired valency and/or length can specifically bind with high affinity to both the internalizing receptor and a target autoantibody simultaneously and exhibit high uptake activity of the target autoantibody. The conjugates of this disclosure can provide for sequestering and degrading of a target autoantibody in the cell's lysosome.

This disclosure thus includes autoantibody degrading bifunctional molecules of formula (I):

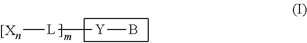

or a prodrug thereof, or a pharmaceutically acceptable salt thereof, wherein:
X is a moiety that binds to a lysosomal targeting molecule;
n is 1 to 20 (e.g., 1 to 10, 1 to 6, or 1 to 4 for M6PR ligands, or e.g., 1 to 3 for GalNAc ligands);
L is a linker:
m is 1 to 10 (e.g., 1 to 6, or 1 to 4, where m can be a discrete loading or an average loading (i.e. ligand to polypeptide (LPR) ratio));
Y is an optional carrier polypeptide; and
B is a polypeptide that specifically binds an extracellular target molecule (e.g., autoantibody).

In some embodiments of formula (I), the bifunctional molecule is of formula (Ia):

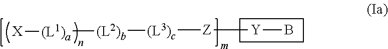

wherein:
Z is residual moiety resulting from the covalent linkage of a chemoselective ligation group of the linker to a compatible group of Y—B;
the linker L is comprised of optional linking moieties L¹, L² and L³ that together provide a linear or branched linker between X and Y—B, wherein L¹ and L³ are independently a linear linking moiety, and L² is a branched linking moiety, wherein a, b and c are independently 0 or 1;
n is 1, 2, or 3, wherein:
   when n is 1, b is 0 and at least one of a and c is 1; and
   when n is 2 or 3, a, b and c are each 1; and
m is the average number of (Xₙ-L) moieties conjugated to Y—B, wherein m is in the range from about 1 to about 80 (e.g., m is 1 to 20, 1 to 10, 1 to 8, 2 to 8, 3 to 6, or 4 to 5, or m is from 1 to 3, such as 1, 2 or 3).

In certain embodiments of formula (Ia), Z is a residual moiety resulting from the covalent linkage of a thiol-reactive chemoselective ligation group to one or more cysteine residue(s) of Y—B; or Z is a residual moiety resulting from the covalent linkage of an amine-reactive chemoselective ligation group to one or more lysine residue(s) of Y—B.

In some embodiments target binding conjugate is of formula (II′):

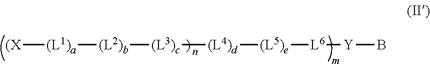

or a prodrug thereof, or a salt thereof,
wherein:
n is 1 to 3:
m is 1 to 3;
X, Y, and B are each independently as defined herein;
each L¹ to L⁶ is independently a linking moiety which together provide a linear or branched linker between Z¹ and Y; and
a, b, c, d, and e are each independently 1, 2, 3, 4, or 5.

In certain embodiments of formula (I)-(Ia), Y is absent and the linker is connected directly to polypeptide B. In certain embodiments of formula (I)-(Ia), a carrier polypeptide Y is present and the linker is connected directly to Y and/or polypeptide B.

In certain embodiments of formula (Ia), Y—B is a chimeric fusion protein including a carrier polypeptide and a polypeptide that specifically binds the target autoantibody.

Each of the components of the lysosomal targeting bifunctional molecules, preferred configurations of such molecules, and methods of using the same, are now described in greater detail.

### Lysosomal Targeting Moiety

A lysosomal targeting receptor is a cell surface receptor that provides for internalization of the conjugate compounds of this disclosure.

In some embodiments, the lysosomal targeting receptor is selected from asialoglycoprotein receptor (ASGPR), cation independent mannose-6-phosphate receptor (CI-M6PR also referred to herein as M6PR), folate receptor, CD63, sortilin, IFITM3, molecules in the endosome/lysosome pathway, LIMP-1, and LIMP-2. In some embodiments, the lysosomal targeting receptor is ASGPR. In some embodiments, the lysosomal targeting receptor is CI-M6PR. In some embodiments, the lysosomal targeting receptor is folate receptor.

A variety of ligand moieties that binds to a lysosomal targeting molecule can be utilized in the conjugate compounds of this disclosure.

ASGPR binding compounds and conjugates are described in International Publication WO2023/288033, filed Jul. 14, 2022, the disclosure of which is herein incorporated by reference in its entirety.

CI-M6PR binding compounds and conjugates are described in International Publication WO2023/288015, filed Jul. 14, 2022, the disclosure of which is herein incorporated by reference in its entirety.

ASGPR binding compounds and conjugates, or CI-M6PR binding compounds and conjugates are described in International Publication WO2022/142377, filed Jan. 8, 2021, the disclosure of which is herein incorporated by reference in its entirety.

Folate receptor binding compounds and conjugates are described in International Publication WO2022/150721, filed Jan. 10, 2022, the disclosure of which is herein incorporated by reference in its entirety.

### ASGPR Ligands

An asialoglycoprotein receptor (ASGPR) ligand moiety is a moiety that binds to ASGPR (i.e., also referred to as an ASGPR binding moiety) and, via the ASGPR, facilitates internalization of the bifunctional molecule of which it is a part, plus any bound target molecule, e.g., autoantibody. The ASGPR ligand moieties of this disclosure can be connected via a linker to a polypeptide construct without impacting the specific binding to, or function of, the cell surface ASGPR. The inventors have demonstrated that lysosomal targeting bifunctional molecules of this disclosure which include one or more linked ASGPR ligand moieties can utilize the functions of cell surface ASGPRs in a biological system, e.g., for internalization and/or sequestration of autoantibody to the lysosome of a cell, and subsequent lysosomal degradation, e.g., in the methods of this disclosure.

ASGPR, also known as the Ashwell Morell receptor, is a transmembrane glycoprotein receptor found primarily in hepatocytes which mediates the endocytosis and lysosomal degradation of glycoproteins with exposed terminal galactose or N-acetylgalactosamine (GalNAc) residues. ASGPR cycles between intracellular endosomes and the cell surface. In some embodiments, the ASGPR is Homo sapiens asialoglycoprotein receptor 1 (ASGR1) (see, e.g., NCBI Reference Sequence: NM_001197216).

In some embodiments, the ASGPR binding moiety (X) includes an amino sugar ring analog of galactose (e.g., N-acetylgalactosamine, or analogs thereof) that is connected to a linker scaffold via an optional linking moiety at the 1-, 2- or 6-position of the sugar ring analog. In some embodiments, the linking moiety includes an oxygen, sulfur, nitrogen or carbon atom connected at the 1-position of the ring. In some embodiments, the linking moiety includes an oxygen, sulfur, nitrogen or carbon atom connected at the 2-position of the ring. In some embodiments, the linking moiety includes an oxygen, sulfur, nitrogen or carbon atom connected at the 1-position of the ring. In certain embodiments, the linking moiety connected at the 1-, 2-, or 6-position of the ring includes an optionally substituted aryl or heteroaryl group. In certain embodiments, the amino sugar ring analog of galactose has a bicyclic structure.

ASGPR ligand moieties of interest which can be adapted for use in the conjugates of this disclosure are described in International Application No. WO 2023/288033, filed Jul. 14, 2022, the disclosure of which is herein incorporated by reference in its entirety. Preferred ASGPR ligand moieties of interest are described below.

In some embodiments, the ASGPR ligand moieties (e.g., (X-L)ₙ of formula (I)) of the bifunctional molecule specifically bind to ASGPR with an affinity (K_{d}) of 300 nM or less, such as 100 nM or less, 30 nM or less, 10 nM or less, 3 nM or less, or 1 nM or less. The terms “binds,” “binds to,” “specifically binds” or “specifically binds to” in this context are used interchangeably.

In some embodiments of the conjugates described herein, X is an asialoglycoprotein receptor (ASGPR) binding moiety of formula (II):

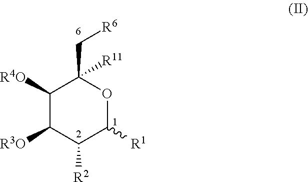

wherein:
R¹ is selected from —Z¹—*, —H, —OH, optionally substituted (C₁-C₆)alkyl, —OCH₃, —OCH₂CH═CH, optionally substituted —S—(C₁-C₆)alkyl, optionally substituted aryl, optionally substituted heteroaryl, optionally substituted —S-aryl, and optionally substituted —S-heteroaryl;
R² is selected from —Z¹—*, —NHCOCH₃, —NHCOCF₃, —NHCOCH₂CF₃, —OH, —NHR, and optionally substituted triazole;
R⁶ is selected from —Z¹—*, —OH, —OR, optionally substituted (C₁-C₆)alkyl, —OC(O)R, —C(O)NHR, —NR^{xx}R^{yy}, optionally substituted aryl, optionally substituted heteroaryl, —NHCOR, and —NRCOR;
each R is independently optionally substituted (C₁-C₆)alkyl, optionally substituted aryl, or optionally substituted heteroaryl;
R^{xx} and R^{yy} are independently H, optionally substituted (C₁-C₆)alkyl, or R^{xx} and R^{yy} can cyclize to form an optionally substituted heterocyclyl;
wherein one of R¹, R², and R⁶ is —Z¹—*, and “*” represents a point of connection of Z¹ to the linker (L);
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring;
Z¹ is a linking moiety selected from —Z¹¹—, —Z¹¹-A¹-, -A²-, —NR²¹CO—, —CONR¹—, —NR²¹SO₂—, —SO₂NR²¹—, —NR²¹C(═O)NR²¹—, and —NR²¹C(═S)NR²¹—;
—Z¹¹— is —O—, —S—, —N(R²¹)—, or —C(R²²)₂;
-A¹- and -A²- are optionally substituted heterocyclylene, optionally substituted arylene, or optionally substituted heteroarylene;
each R²¹ is independently selected from H, optionally substituted (C₁-C₆)alkyl, —COR, and optionally substituted heteroaryl; and
each R²² is independently selected from H, halogen, and optionally substituted (C₁-C₆)alkyl.

In some embodiments, X is represented by formula (a-II):

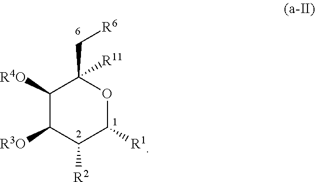

In some embodiments, the lysosomal targeting bifunctional molecules of this disclosure (e.g., of formula (I)-(Ia)) can include an ASGPR ligand moiety of formula (II):

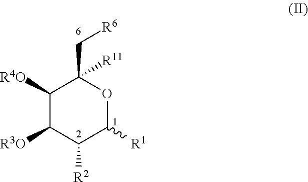

wherein:
R¹ is selected from —Z¹—*, —H, —OH, —CH₃, —OCH₃, and —OCH₂CH═CH;
R² is selected from —Z¹—*, —NHCOCH₃, —NHCOCF₃, —NHCOCH₂CF₃, —OH, and optionally substituted triazole;
R⁶ is selected from —Z¹—*, —OH, —OC(O)R, —C(O)NHR, and optionally substituted triazole, where R is optionally substituted (C₁-C₆)alkyl or optionally substituted aryl:
wherein one of R¹, R², and R⁶ is —Z¹—*, and “*” represents a point of connection of Z¹ to the linker (L);
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring:
Z¹ is a linking moiety selected from —Z¹¹—, —Z¹¹-A¹-, -A²-, —NR²¹CO—, —CONR²¹—, —NR²¹SO₂—, —SO₂NR²¹—, —NR²¹C(═O)NR²¹—, and —NR²¹C(═S)NR²¹—;
—Z¹¹— is —O—, —S—, —N(R²¹)—, or —C(R²²)₂;
-A¹- and -A²- are optionally substituted arylene or optionally substituted heteroarylene;
each R²¹ is independently selected from H, and optionally substituted (C₁-C₆)alkyl; and
each R²² is independently selected from H, halogen (e.g., F) and optionally substituted (C₁-C₆)alkyl.

In some embodiments of formula (II):
i) when n is 3, R6 is OH, R2 is —NHCOCH3, R3-R4 are H, and R1 is Z1, then Z1 is not O;
ii) when n is 2 or 3, R⁶ is OAc, R² is —NHCOCH₃, R³-R⁴ are Ac, and R¹ is Z¹, then Z¹ is not O;
iii) when n is 2 or 3, R⁶ is Obz, R² is —NHCOCH₃, R³-R⁴ are Bz, and R¹ is Z¹, then Z¹ is not O;
iv) when n is 3, R⁶ is OH, R² is —NHCOCH₃, R³-R⁴ are H, and R¹ is Z¹, and Z¹¹ is O, then L comprises a backbone of at least 16 consecutive atoms to a branching point;
v) when n is 3, R⁶ is Z¹, where Z¹ is O, and R³-R⁴ are H, then R¹ is not —CH₃—OCH₃, or —OCH₂CH═CH; and
vi) when R¹¹ is a group of the formula —CH₂O— that forms a bridge (i.e., is cyclically linked) to the 1-position carbon atom on the sugar ring, R² is —NHCOCH₃, R³-R⁴ are H, then R¹ and R³ are not Z¹.

In some embodiments, R¹ is —Z¹—*, —H, or (C₁-C₆)alkyl. In some embodiments. R¹ is —Z¹—*, —H, or n-propyl.

In some embodiments, R² is —Z¹—* or —NHCOCH₃.

In some embodiments, R³ and R⁴ are each —H.

In some embodiments, L comprises of 10 to 60 consecutive branched or linear chain atoms.

In some embodiments, L is of formula (IIb′):

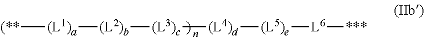

wherein:
each L¹ to L⁶ is independently a linking moiety which together provide a linear or branched linker between Z¹ and Y:
a, b, c, d, and e are each independently 1, 2, 3, 4, or 5;
** represents the point of attachment to L¹ of X via Z¹; and
*** represents the point of attachment to Y.

In some embodiments, L is of formula (IIb′):

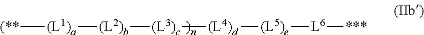

wherein:
n is 1, 2, or 3;
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z; and
*** represents the point of attachment to Y.

In some embodiments, each L1 to L5 independently comprises one or more linking moieties independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50;
L⁶ is a linking group comprising one or more linking moieties independently selected from —C₁₋₂₀-alkylene-, —NR¹⁶C(O)—C₁₋₆-alkylene-, —C(O)NR¹⁶—C₁₋₆-alkylene-, —NR¹⁶—C₁₋₆-alkylene-, —NR¹⁶C(O)NR¹⁶—C₁₋₆-alkylene-, —NR¹⁶C(S)NR¹⁶—C₁₋₆-alkylene-, —C₁₋₆-alkylene-NR¹⁶C(O)—, —C₁₋₆-alkylene-C(O)NR¹⁶—, —C₁₋₆-alkylene-NR¹⁶—, —C₁₋₆-alkylene-NR¹⁶C(O)NR¹⁶—, —C₁₋₆-alkylene-NR¹⁶C(S)NR¹⁶—, —O(CH₂)ₚ—, —(OCH₂CH₂)ₚ—, —NR¹⁶C(O)—, —C(O)NR¹⁶—, —NHS(O)₂—, —S(O)₂NH—, —C(O)—, —S(O)₂—, —O—, —S—, monocyclic heteroaryl, monocyclic aryl, monocyclic heterocycle, amino acid residue, or —NR¹⁶—; and
each R¹⁶ is independently —H, optionally substituted (C₁-C₆)alkyl, optionally substituted aryl, optionally substituted monocyclic heteroaryl or monocyclic heteroaryl.

In some embodiments, each L1 to L5 is independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C( )NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein
each L¹ to L⁵ is independently optionally substituted with one to five halo;
each p is independently 1 to 50; and
L⁶ is

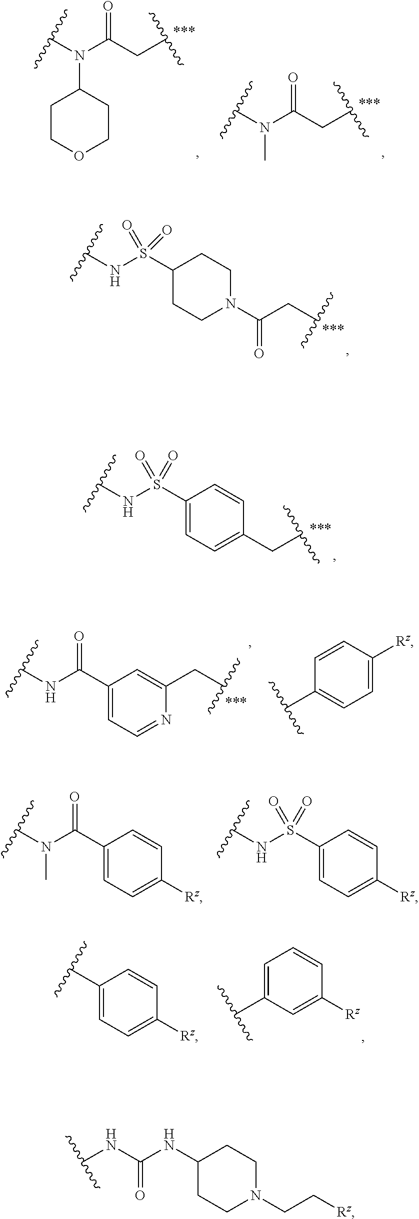

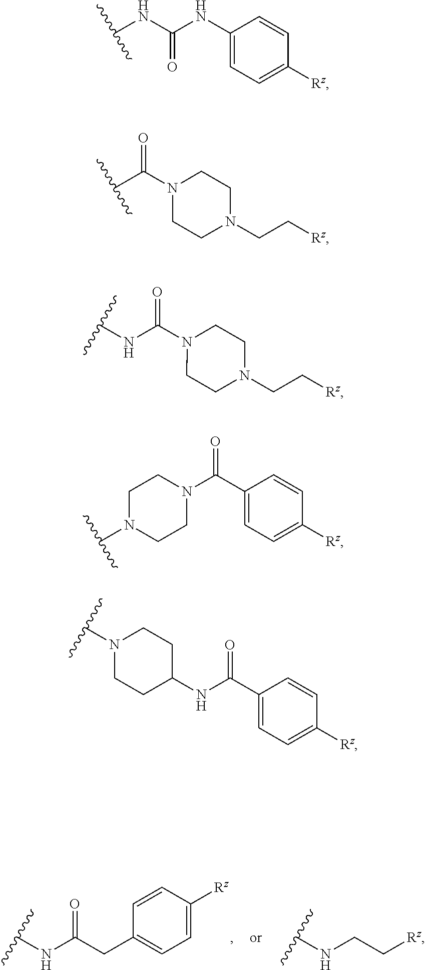

wherein R^{z} is

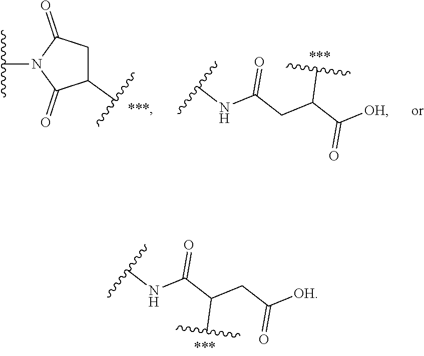 [SMILES: *C1CC(=O)N(*)C1=O ; *NC(=O)CC(*)C(=O)O ; *NC(=O)C(*)CC(=O)O]

### 1-Linked ASGPR Ligand Moieties

In some embodiments, the ASGPR binding moiety (X) of the compounds of this disclosure can be described by formula (IIa):

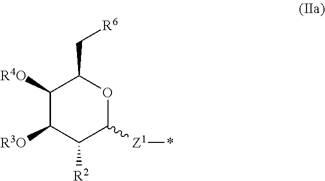

wherein R², R³, R⁴, R⁶ and Z are as defined herein. In some embodiments of formula (IIa), R⁶ is selected from —OH, —OC(O)R, and —C(O)NHR; and R² is selected from NHCOCH₃, NHCOCF₃, and —NHCOCH₂CF₃.

In some embodiments of formula (II), Z¹ is in a beta configuration, and can be described by formula (IIa-1):

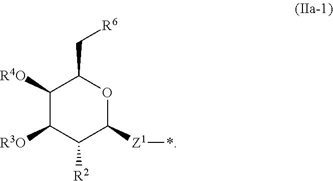

In some embodiments of formula (II), Z¹ is in an alpha configuration, and can be described by formula (IIa-2)

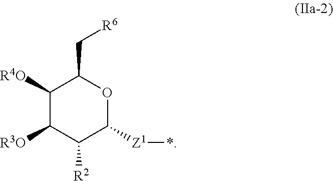

In certain embodiments of formula (IIa), (IIa-1) or (IIa-2), Z¹ is —Z¹¹-A-, wherein A¹- is optionally substituted arylene or optionally substituted heteroarylene. In certain cases, A¹ is an optionally substituted heteroarylene. In certain cases, the heteroarylene is a 5 or 6-membered heteroarylene. In certain cases, the heteroarylene is a 5-membered heteroarylene. In certain cases, the 5-membered heteroarylene is a triazole. In certain cases, the triazole is a 1,2,3-triazole moiety.

In some embodiments, the ASGPR binding moiety (X) of the compounds of this disclosure can be described by formula (IIIa) or (IIIb):

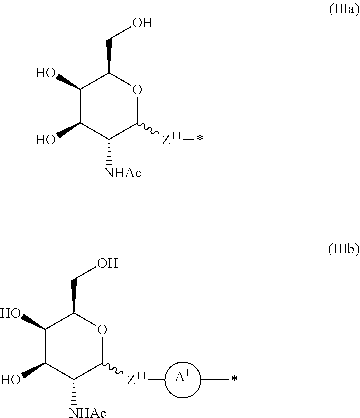

wherein:
—Z¹¹— is —O—, —S—, —N(R²¹)—, or —C(R²²)₂—, where each R²² is independently selected from H, halogen (e.g., F) and optionally substituted (C₁-C₆)alkyl, and R²¹ is H or optionally substituted (C₁-C₆)alkyl; and
-A¹- is arylene, substituted arylene, heteroarylene, or substituted heteroarylene.

In some embodiments of formula (IIIa) or (IIIb), Z¹¹ is —S—.

In some embodiments, Z¹¹ is —C(R²²)₂—. In some embodiments. Z¹¹ is —CH₂—.

In certain embodiments, Z¹¹ is —C(R²²)₂, where at least one R²² is H. In certain cases, both R²² are H. In certain embodiments Z¹¹ is —O—. In certain embodiments, Z¹¹ is —S—. In certain embodiments cases, Z¹¹ is —N(R²¹), where R²¹ is H or (C₁-C₃)alkyl.

In certain embodiments, -A¹- is triazole.

In certain embodiments, Z¹ is —C(R²²)₂-triazole-. In certain embodiments, Z¹ is:

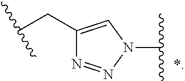 [SMILES: *Cc1cn(*)nn1]

In certain embodiments, Z¹ is:

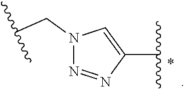 [SMILES: *Cn1cc(*)nn1]

In certain embodiments of formula (IIa), (IIa-1) or (IIa-2). Z¹ is Z¹¹. In certain cases, Z¹¹ is —C(R²²)₂. In certain cases, at least one R²² is H. In certain cases, both R²² are H, and Z¹¹ is —CH₂—. In certain cases Z¹¹ is —O—. In certain cases, Z¹¹ is —S—. In certain other cases, Z¹¹ is —N(R²¹), where R²¹ is H or (C₁-C₃)alkyl.

In certain embodiments of formula (IIa), (IIa-1) or (IIa-2), Z¹ is monocyclic 5 or 6-membered heteroaryl or aryl. In certain cases, Z¹ is

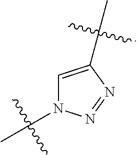 [SMILES: *c1cn(*)nn1]

In certain cases, Z¹ is

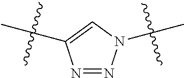 [SMILES: *c1cn(*)nn1]

In certain embodiments of formula (IIa) (IIa-1) or (IIa-2), Z¹ is selected from —O—, —S—, —C(R²²)₂—, —NR²¹—, —CONR²¹— and

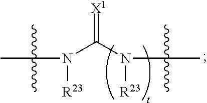

wherein:
X¹ is O or S:
t is 0 or 1;
R²¹ and each R²³ is independently selected from H, and optionally substituted (C₁-C₆)alkyl (e.g., CO₃)-alkyl, such as methyl); and
each R²² is independently selected from H, halogen (e.g., F) and optionally substituted (C₁-C₆)alkyl.

In certain embodiments of formula (IIa), (IIa-1), or (IIa-2), Z¹ is optionally substituted (C₁-C₆)alkyl. In some cases of Z¹ the alkyl is methyl. In some cases of Z¹ the alkyl is ethyl. In some cases of Z¹, the alkyl is propyl. In some cases of Z¹, the alkyl is butyl. In some cases of Z¹, the alkyl is pentyl. In some cases of Z¹, the alkyl is hexyl.

In certain embodiments, the ASGPR binding moiety (X) of formula (IIa-1) is selected from one of the following structures:

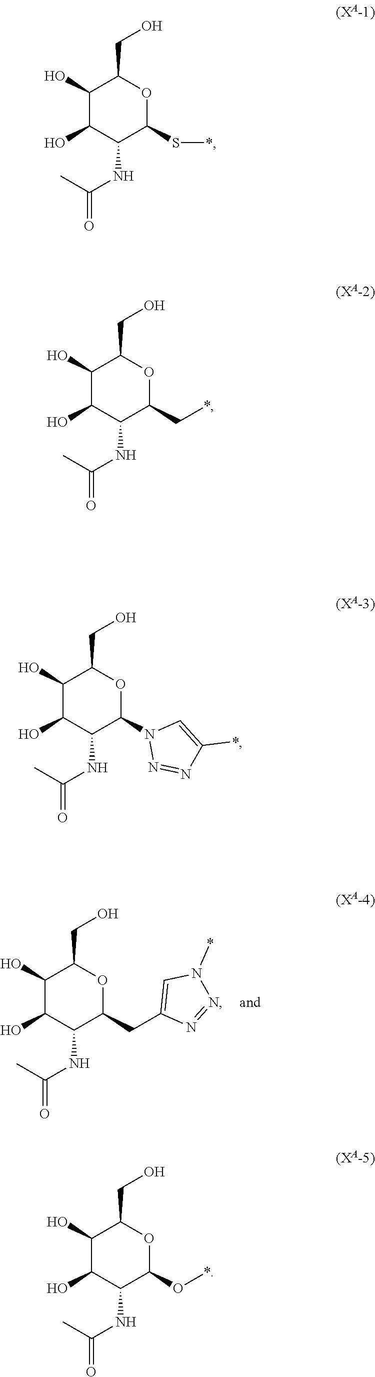

In some embodiments of formula (IIa-2). Z¹ is in a beta configuration and X is of formula (IIIb-2):

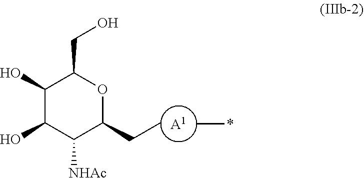

wherein: -A¹- is arylene, substituted arylene, heteroarylene, or substituted heteroarylene.

In some embodiments of formula (IIIb-2), A¹ is a triazole. In some embodiments of formula (IIIb-2), X is of formula (XA-4)

In some embodiments of formula (IIa-1), Z¹ is in a beta configuration at the 1-position carbon of the galactosamine ring. In some embodiments of formula (IIa-1), Z¹ is S, and each X is of formula (X^{A}-1). In some embodiments of formula (IIa-1), each X is of formula (X^{A}-2). In some embodiments of formula (IIa-1), each X is of formula (X^{A}-3). In some embodiments of formula (IIa-1), each X is of formula (X^{A}-4). In some embodiments of formula (IIa-1), each X is of formula (X^{A}-5).

In certain embodiments, the compound of formula (IIa-2) is selected from one of the following structures:

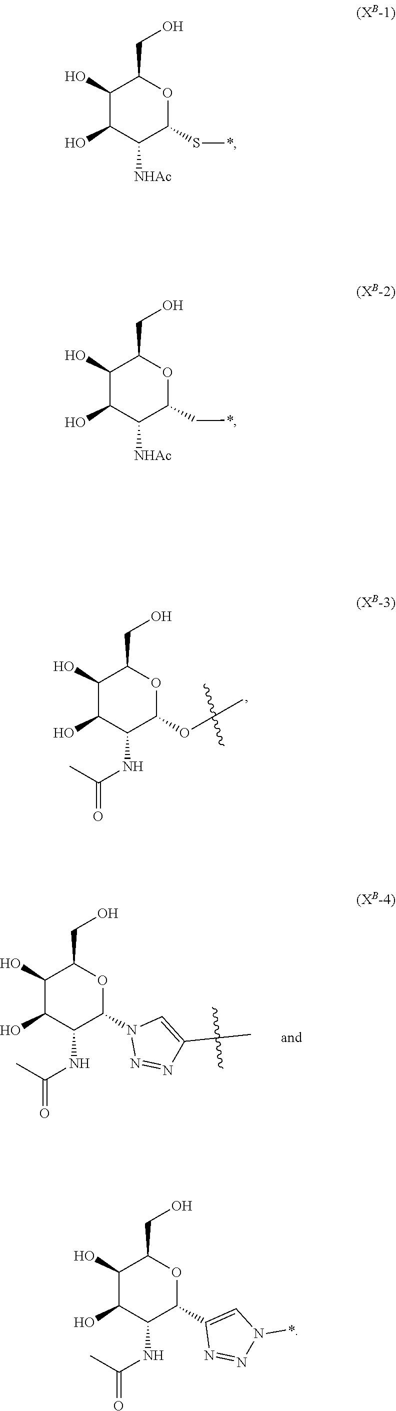

In some embodiments of formula (IIa-2), each X is of formula (X^{B}-1).

In some embodiments of formula (IIa-2), each X is of formula (X^{B}-2).

In some embodiments of formula (IIa-2), each X is of formula (X^{B}-3).

In some embodiments of formula (IIa-2), each X is of formula (X^{B}-4).

In some embodiments of formula (IIa-2), Z¹ is in an alpha configuration and X is of formula (IIIb-1):

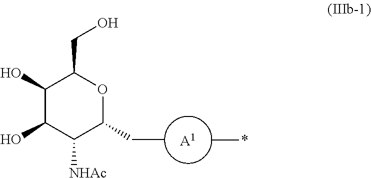

wherein -A¹- is arylene, substituted arylene, heteroarylene, or substituted heteroarylene.

In certain embodiments of formula (IIIb-1), A¹ is an optionally substituted heteroarylene. In certain cases, the heteroarylene is a 5 or 6-membered heteroarylene. In certain cases, the heteroarylene is a 5-membered heteroarylene. In certain cases, the 5-membered heteroarylene is a triazole. In certain cases, the triazole is a 1,2,3-triazole moiety.

In certain embodiments, the X of formula (IIIb-1) is selected from one of the following structures:

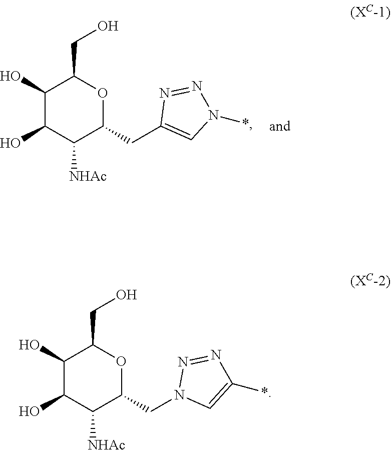

In some embodiments of formula (IIIb-1), each X is of formula (X^{C}-1).

In some embodiments of formula (IIIb-1), each X is of formula (X^{C}-2).

Exemplary ligand moieties that bind ASGPR, and synthons thereof, which can be utilized in the compounds of this disclosure are shown in Tables 1-5. In certain embodiments, the compound of formula (IIa) is a compound shown in Table 1:

TABLE 1
Exemplary ASGPR binding moieties (X) of formula (IIa)

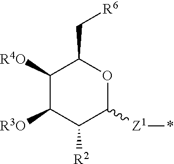

| # | R⁶ | R² | R⁴ | R³ | Z¹ |
|---|---|---|---|---|---|
| X1 | —OH | —NHC(O)CH₃ | H | H | —O— |
| X2 | —OH | —NHC(O)CH₃ | H | H | —S— |
| X3 | —OH | —NHC(O)CH₃ | H | H | —CH₂— |
| X4 | —OH | —NHC(O)CH₃ | H | H | 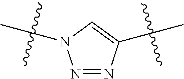 [SMILES: *c1cn(*)nn1] |
| X4.1 | —OH | —NHC(O)CH₃ | H | H | 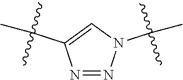 [SMILES: *c1cn(*)nn1] |
| X5 | —OH | —NHC(O)CH₃ | H | —C(O)CH(CH₃)₂ | —O— |
| X5.1 | —OH | —NHC(O)CH₃ | H | H | —NH— |

In some embodiments of any one of X1-X5.1, Z¹ is in the alpha configuration such that the ASGPR binding moiety X1-X5.1 is derived from formula (IIa-2):

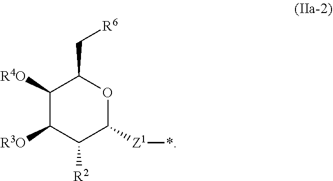

### 2-Linked ASGPR Ligand Moieties

In some embodiments, the ASGPR binding moiety (X) is linked via the 2-postion of the sugar analog. In some embodiments, the ASGPR binding moiety (X) has a reduced ring carbon at the 1-position relative to a galactosamine derived sugar. In some embodiments, the ASGPR binding moiety (X) of the bifunctional molecules of this disclosure is described by formula (IIb):

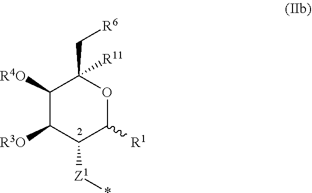

wherein R¹, R³, R⁴, R⁶, R¹¹, and Z¹ are as defined herein.

In some embodiments, the ASGPR binding moiety (X) of the compounds of this disclosure are described by formula (IIb′):

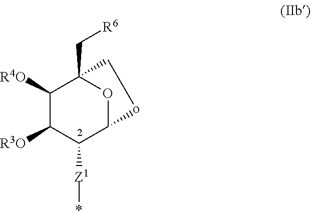

wherein R³-R⁴, R⁶, and Z¹ are as defined herein.

In some embodiments, the ASGPR binding moiety (X) of the compounds of this disclosure are described by formula (IVa):

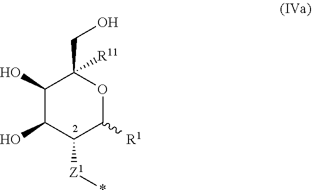

wherein R¹, R¹¹, and Z¹ are as defined herein.

In some embodiments of formulae (IIb), (IIb′) or (IVa), Z¹ is selected from optionally substituted —(C(R²²)₂)_{q}-heteroarylene, and

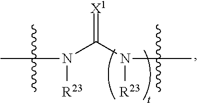

wherein q is 0 or 1.

In some embodiments of formulae (IIb), (IIb′) or (IVa). Z¹ is optionally substituted —(C(R²²)₂)_{q}-triazole wherein q is 0 or 1.

In some embodiments of formulae (IIb), (IIb′) or (IVa), Z¹ is

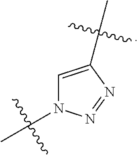 [SMILES: *c1cn(*)nn1]

In some embodiments, Z¹ is

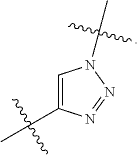 [SMILES: *c1cn(*)nn1]

In some embodiments of formulae (IIb), (IIb′) or (IVa), Z¹ is

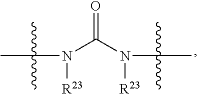

wherein R²³ is H, or C₍₁₋₃₎-alkyl.

In some embodiments of formulae (IIb), (IIb′) or (IVa), Z¹ is —NR²³CO—, wherein R²³ is H or C₍₁₋₃₎-alkyl.

In certain embodiments of formula of formulae (IIb), (IIb′) or (IVa), Z¹ is selected from optionally substituted —(C(R²²)₂)_{q}-heteroaryl, and

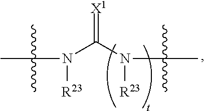

wherein q is 0 or 1.

In certain embodiments of formula of formulae (IIb), (IIb′) or (IVa), Z¹ is optionally substituted —(C(R²²)₂)_{q}-triazole wherein q is 0 or 1. In certain cases, Z¹ is

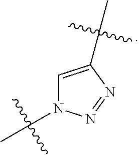 [SMILES: *c1cn(*)nn1]

In certain cases of formulae (IIb), (IIb′) or (IVa), Z¹ is

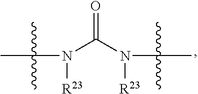

wherein R²³ is H, or C₍₁₋₃₎-alkyl.

In certain cases of formulae (IIb), (IIb′) or (IVa), Z¹ is —NR²³CO—, wherein R²³ is H or C₍₁₋₃₎-alkyl.

In certain embodiments of formula of formulae (IIb), (IIb′) or (IVa), Z¹ is monocyclic 5 or 6-membered heteroarylene or arylene. In certain cases, Z¹ is

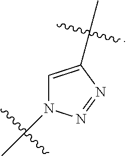 [SMILES: *c1cn(*)nn1]

In certain embodiments of formula of formulae (IIb), (IIb′) or (IVa), Z¹ is selected from —O—, —S—, —C(R²²)₂—, —NR²¹—, —CONR²¹—, and

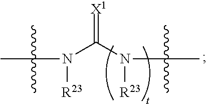

wherein:
X¹ is O or S:
t is 0 or 1:
R²¹ and each R²³ is independently selected from H, and optionally substituted (C₁-C₆)alkyl (e.g., C₍₁₋₃₎-alkyl, such as methyl); and
each R²² is independently selected from H, halogen (e.g., F) and optionally substituted (C₁-C₆)alkyl.

In certain embodiments, the compound of formula of formulae (IIb), (IIb′) or (IVa) is selected from one of the following structures:

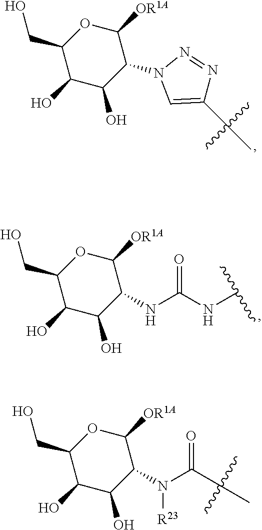

wherein R^{1A} is independently H or (C₁₋₃)alkyl.

In some embodiments, the ASGPR binding moiety (X) of the compounds of this disclosure can be described by formula (IVb) or (IVc):

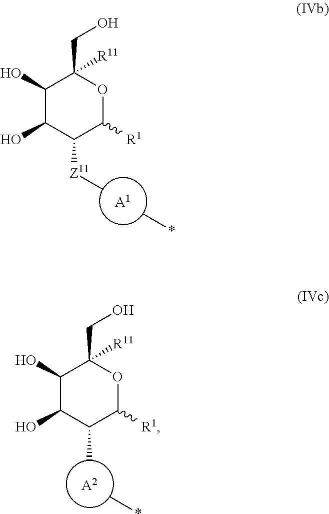

wherein:
—Z¹¹— is —O—, —S—, —N(R²¹)—, or —C(R²²)₂;
-A¹- and -A²- are optionally substituted arylene or optionally substituted heteroarylene;
each R²¹ is independently selected from H, and optionally substituted (C₁-C₆)alkyl; and
each R²² is independently selected from H, halogen (e.g., F) and optionally substituted (C₁-C₆)alkyl. In some embodiments of formula (IVb) or (IVc), R¹ is H.

In some embodiments, —Z¹—* or —Z¹-L- comprises

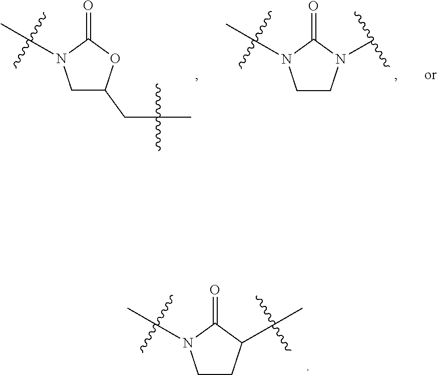 [SMILES: *CC1CN(*)C(=O)O1 ; *N1CCN(*)C1=O ; *C1CCN(*)C1=O]

In certain embodiments of formula (IIb), R¹¹ is H and the compound is of Table 2:

TABLE 2
Exemplary ASGPR binding moieties (X) of formula (IIb)

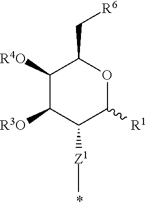

| # | R⁶ | R¹ | R⁴ | R³ | Z¹ |
|---|---|---|---|---|---|
| X9 | —OH | —OCH₃ | H | H | 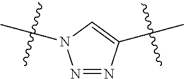 [SMILES: *c1cn(*)nn1] |
| X10 | —OH | —OCH₃ | H | H | —NH(CO)NH— |
| X11 | —OH | —OCH₃ | H | H | —NHC(O)— |

In certain embodiments, the compound of formula (Iib) is a compound shown in Table 3; In certain embodiments, the compound of formula (Iib), the configuration at C1 (i.e., R¹) is alpha. In certain embodiments, the compound of formula (Iib), the configuration at C1 (i.e., R¹) is beta.

TABLE 3
Other exemplary ASGPR binding moieties (X) of formula (Iib)

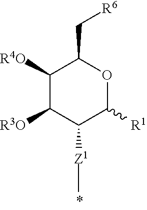

| # | R⁶ | R¹ | R⁴ | R³ | Z¹ |
|---|---|---|---|---|---|
| X12 | —OH | H | H | H | 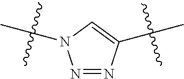 [SMILES: *c1cn(*)nn1] |
| X13 | —OH | H | H | H | 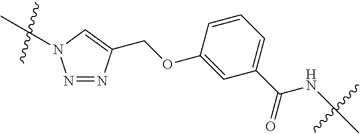 [SMILES: *NC(=O)c1cccc(OCc2cn(*)nn2)c1] |
| X14 | —OH | H | H | H | 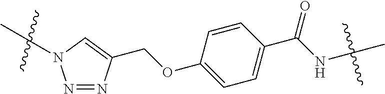 [SMILES: *NC(=O)c1ccc(OCc2cn(*)nn2)cc1] |
| X15 | —OH | H | H | H | —CH₂— |
| X16 | —OH | H | H | H | 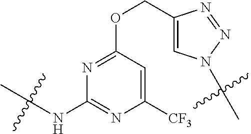 [SMILES: *Nc1nc(OCc2cn(*)nn2)cc(C(F)(F)F)n1] |
| X17 | —OH | H | H | H | 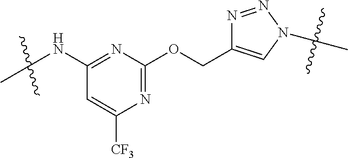 [SMILES: *Nc1cc(C(F)(F)F)nc(OCc2cn(*)nn2)n1] |
| X18 | —OH | H | H | H | 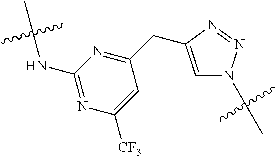 [SMILES: *Nc1nc(Cc2cn(*)nn2)cc(C(F)(F)F)n1] |
| X19 | —OH | H | H | H | 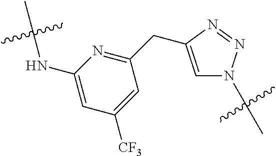 [SMILES: *Nc1cc(C(F)(F)F)cc(Cc2cn(*)nn2)n1] |
| X20 | —OH | H | H | H | 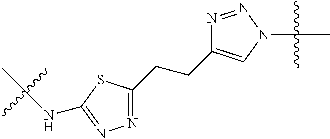 [SMILES: *Nc1nnc(CCc2cn(*)nn2)s1] |
| X21 | —OH | H | H | H | 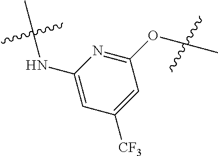 [SMILES: *Nc1cc(C(F)(F)F)cc(O*)n1] |
| X22 | —OH | H | H | H | 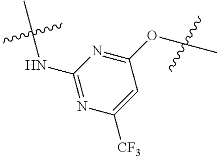 [SMILES: *Nc1nc(O*)cc(C(F)(F)F)n1] |
| X23 | —OH | H | H | H | 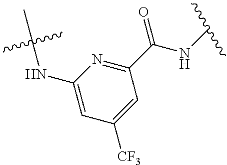 [SMILES: *NC(=O)c1cc(C(F)(F)F)cc(N*)n1] |
| X24 | —OH | H | H | H | —O— |

In certain embodiments, the compound of formula (Id′) is a compound shown in Table 4:

TABLE 4
Other exemplary ASGPR binding moieties (X) of formula (Iib′)

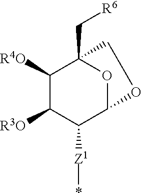

| # | R¹ | R⁴ | R⁵ | Z¹ |
|---|---|---|---|---|
| X25 | —OH | H | H | 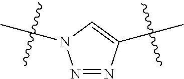 [SMILES: *c1cn(*)nn1] |

In some embodiments, the ASGPR binding moiety (X) of the compounds of this disclosure can be described by formula (IVb-1) or (IVc-1):

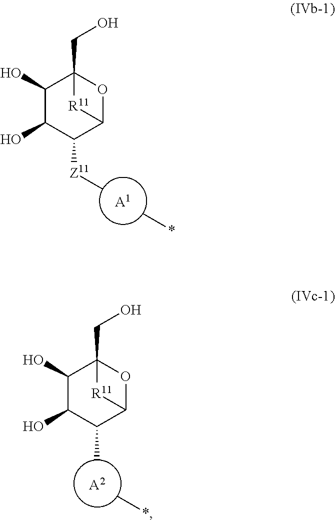

wherein R¹¹ is the bridging moiety that connects the 5-position carbon to the 1-position carbon.

In some embodiments of formulae (IVb), or (IVb-1), Z¹¹ is —C(R²²)₂. In certain cases, at least one R²² is H. In certain cases, both R²² are H. In certain cases, Z¹¹ is —O—. In certain cases, Z¹¹ is —S—. In certain cases, Z¹¹ is —N(R²¹), where R²¹ is H or (C₁-C₃)alkyl.

In certain embodiments of formulae (IVb), (IVc), (IVb-1) or (IVc-1), -A¹- and -A²- are each independently an optionally substituted heteroarylene. In certain cases, the heteroarylene is a 5 or 6-membered heteroarylene. In certain cases, the heteroarylene is a 5-membered heteroarylene. In certain cases, the heteroarylene is a 6-membered heteroarylene.

In some embodiments of formulae (IVb), or (IVb-1), the A¹ ring is a 5-membered heteroarylene selected from triazole, thiadiazole, thiophene, oxazole, isoxazole, isothiazole, thiazole, oxadiazole, and furan. In certain cases, the A¹ ring is a 6-membered heteroarylene selected from pyridine, pyrimidine, pyridazine, pyrazine, and triazine. In certain cases, the A¹ ring is triazole. In certain cases, the A¹ ring is pyridine. In certain cases, the A¹ ring is pyrimidine. In certain cases, the A¹ ring is thiadiazole. In certain cases, the A¹ ring is a 5 or 6-membered arylene or heteroarylene that is further substituted with one or more substituents. In some cases, the A¹ ring is further substituted with one or more substituents selected from halogen, (C₁-C₆)alkyl and substituted (C₁-C₆)alkyl (e.g., CF₃).

In some embodiments of any one of formulae (IVc), or (IVc-1), the A² ring is a 5-membered heteroarylene selected from triazole, thiadiazole, thiophene, oxazole, isoxazole, isothiazole, thiazole, oxadiazole, and furan. In certain cases, the A² ring is a 6-membered heteroarylene selected from pyridine, pyrimidine, pyridazine, pyrazine, and triazine. In certain cases, the A² ring is triazole. In certain cases, the A² ring is pyridine. In certain cases, the A² ring is pyrimidine. In certain cases, the A² ring is thiadiazole. In certain cases, the A² ring is a 5 or 6-membered arylene or heteroarylene that is further substituted with one or more substituents. In some cases, the A² ring is further substituted with one or more substituents selected from halogen, (C₁-C₆)alkyl and substituted (C₁-C₆)alkyl (e.g., CF₃).

In certain embodiments of formulae (IVb) or (IVb-1), —Z¹¹-A¹- is a monocyclic 5 or 6-membered heteroarylene of one of the following structures:

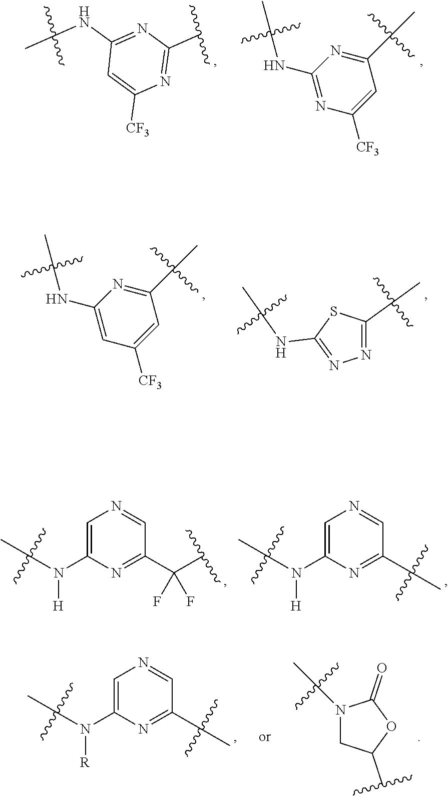

In certain embodiments of formulae (IVc) or (IVc-1), -A²- is a monocyclic 5 or 6-membered heteroarylene of the following structure:

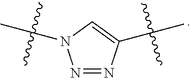 [SMILES: *c1cn(*)nn1]

It is understood that a variety of substituents can be utilized to connect a particular —Z¹¹-A¹- group to an adjacent linker. In certain embodiments of formulae (IVb) or (IVb-1), —Z¹¹-A¹- is a monocyclic 5 or 6-membered heteroarylene that is attached to a linking moiety as shown in one of the following structures:

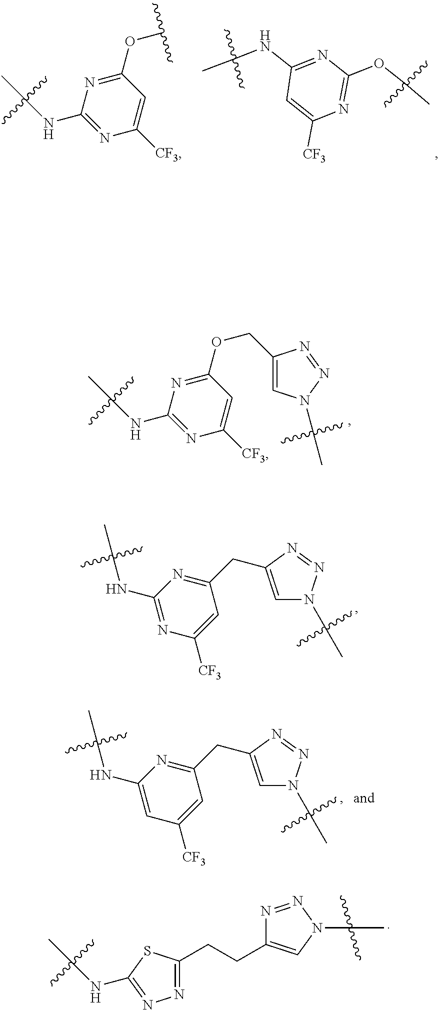

In certain embodiments of formulae (IVc) or (IVc-1), —Z¹¹-A¹- is a monocyclic 5 or 6-membered heteroarylene that is attached to a linking moiety as shown in one of the following structures:

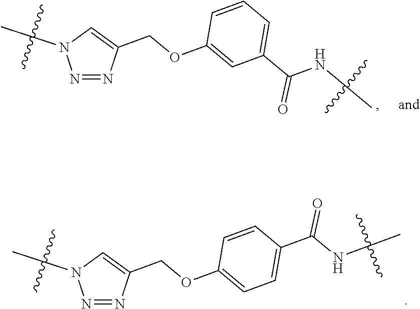 [SMILES: *NC(=O)c1cccc(OCc2cn(*)nn2)c1 ; *NC(=O)c1ccc(OCc2cn(*)nn2)cc1]

In some embodiments of the compound of formula of formulae (IIb), or (IVa)-(IVc)R¹ is H, such that the compound of formula of formulae (IIb), or (IVa)-(IVc) has no non-hydrogen substituents at the 1-position of the sugar ring.

In some embodiments, the compound of formula (IIb) is of any one of formulae (IVd)-(IVg):

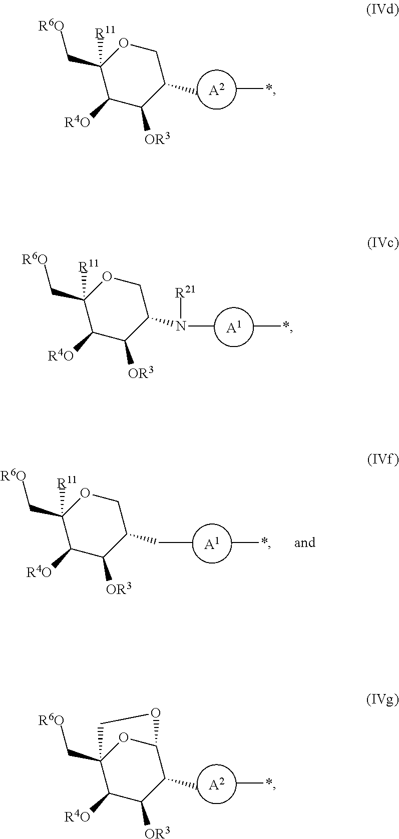

wherein the A¹ and A² rings, R⁶, R⁴, R³, R¹¹, and R²¹ are as defined herein.

In some embodiments of any one of formulae (IVd)-(IVg), the A¹ ring is a 5 or 6-membered arylene or heteroarylene. In certain cases, the A¹ ring is a 5-membered heteroarylene selected from triazole, thiadiazole, thiophene, oxazole, isoxazole, isothiazole, thiazole, oxadiazole, and furan. In certain cases, the A¹ ring is a 6-membered heteroarylene selected from pyridine, pyrimidine, pyridazine, pyrazine, and triazine. In certain cases, the A¹ ring is triazole. In certain cases, the A¹ ring is pyridine. In certain cases, the A¹ ring is pyrimidine. In certain cases, the A¹ ring is thiadiazole. In certain cases, the A¹ ring is a 5 or 6-membered arylene or heteroarylene that is further substituted with one or more substituents. In some cases, the A¹ ring is further substituted with one or more substituents selected from halogen, (C₁-C₆)alkyl and substituted (C₁-C₆)alkyl (e.g., CF₃).

In some embodiments of any one of formulae (IVd)-(IVg), the A² ring is a 5 or 6-membered arylene or heteroarylene. In certain cases, the A² ring is a 5-membered heteroarylene selected from triazole, thiadiazole, thiophene, oxazole, isoxazole, isothiazole, thiazole, oxadiazole, and furan. In certain cases, the A² ring is a 6-membered heteroarylene selected from pyridine, pyrimidine, pyridazine, pyrazine, and triazine. In certain cases, the A² ring is triazole. In certain cases, the A² ring is pyridine. In certain cases, the A² ring is pyrimidine. In certain cases, the A² ring is thiadiazole. In certain cases, the A² ring is a 5 or 6-membered arylene or heteroarylene that is further substituted with one or more substituents. In some cases, the A² ring is further substituted with one or more substituents selected from halogen, (C₁-C₆)alkyl and substituted (C₁-C₆)alkyl (e.g., CF₃).

In some embodiments of any one of formulae (IVd)-(IVg), the A¹ or A² ring is absent.

In some embodiments of any one of formulae (IVd)-(IVg), the A¹ or A² ring is phenylene or substituted phenylene.

In some embodiments of formula (IVd), the A² ring is a 5 or 6-membered heteroarylene. In certain cases of formula (IVd), the A² ring is a 5-membered heteroarylene. In certain embodiments of formula (IVd), the A² ring is triazole. In certain embodiments of (IVd), the A² ring is absent.

In some embodiments of formula (IVe), the A¹ ring is a 5 or 6-membered heteroarylene and R²¹ is H. In certain embodiments of formula (IVe), the A ring is triazole. In certain cases of formula (IVc), the A¹ ring is pyridine. In certain cases of formula (IVe), the A¹ ring is pyrimidine. In certain cases of formula (IVe), the A¹ ring is thiadiazole. In some embodiments of formula (IVe), the A¹ ring is absent and R²¹ is H or optionally substituted acyl. In some cases, R²¹ is —COCH₃. In some cases, R²¹ is H.

In some embodiments of formula (IVf), the A¹ ring is a 5 or 6-membered heteroarylene. In certain cases of formula (IVf), the A¹ ring is a 5-membered heteroarylene. In certain embodiments of formula (IVf), the A¹ ring is triazole. In certain embodiments of (IVf), the A¹ ring is absent.

In some embodiments of formula (IVg), the A² ring is a 5 or 6-membered heteroarylene. In certain cases of formula (IVg), the A² ring is a 5-membered heteroarylene. In certain embodiments of formula (IVg), the A² ring is triazole. In certain embodiments of (IVg), the A² ring is absent.

In some cases, the ASGPR binding moiety (X) of the compounds of this disclosure can be described by any one of formulae (IVh)-(Ivk):

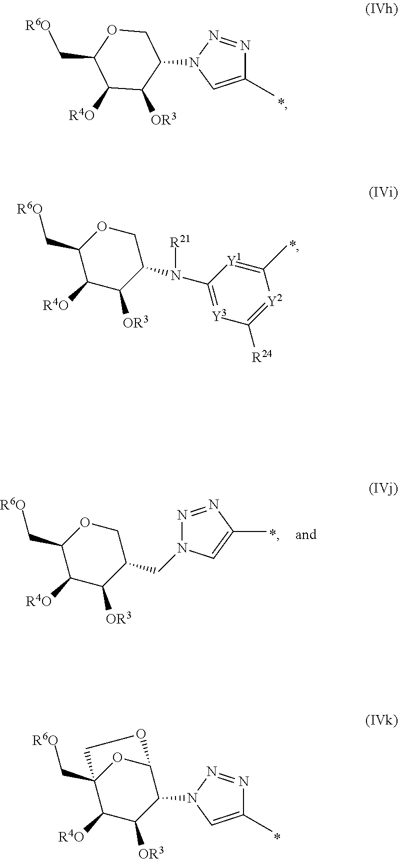

wherein:
R⁶, R⁴, R³, and R²¹ are as defined herein:
Y¹-Y³ are each independently N or CR²⁵; and
R²⁴ and R²⁵ are each independently selected from H, optionally substituted C₍₁₋₆₎-alkyl, optionally substituted fluoroalkyl, and halogen.

In certain embodiments, the ASGPR binding moiety (X) of the compounds of this disclosure can be described by any one of formulae (IVl)-(IVm):

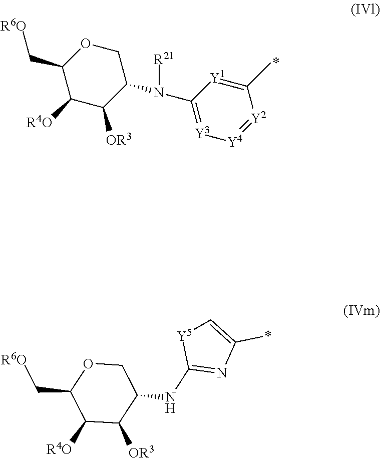

wherein:
R⁶, R⁴, R³, and R²¹ are as defined herein;
Y¹-Y³ are each independently N or CR²⁵;
Y⁵ is N or CR²⁴;
Y⁵ is S, O, or NH; and
R²⁴ and R²⁵ are each independently selected from H, optionally substituted C₍₁₋₆₎-alkyl, optionally substituted fluoroalkyl, and halogen.

In some embodiments of formula (IVi) at least one of Y¹ to Y³ is N. In some cases, at least two of Y¹ to Y³ are N. In certain cases, Y¹ and Y³ are N and Y² is CR²⁵. In certain cases, Y¹ and Y² are N and Y³ is CR²⁵. In certain cases, Y¹ and Y² are CR²⁵ and Y³ is N.

In certain embodiments of any one of formulae (IVd)-(IVk) R⁶ is H.

In some embodiments of any one of formulae (IVd)-(IVk), R⁴ and R³ are each H. In certain cases, at least one of R⁴-R³ is a promoiety. In certain embodiments, R⁴ and R³ are cyclically linked to form a promoiety (e.g., as described herein).

In some embodiments, the compound of formula (IVi) is of formula (IVi-1):

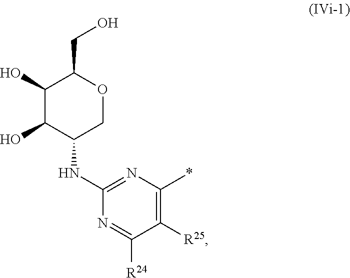

wherein R²⁴ and R²⁵ are independently selected from H, halogen, (C₁-C₆)alkyl and substituted (C₁-C₆)alkyl (e.g., CF₃).

In some embodiments of formula (IVi)-(IVi-1), R²⁵ is H. In certain cases. R²⁵ is C₍₁₋₃₎-alkyl, or C₍₁₋₃₎-fluoroalkyl. In some cases, the fluoroalkyl is CF₃. In some embodiments of formula (IVi) or (IVi-1). R²⁴ is H. In certain cases, R²⁴ is C₍₁₋₃₎-alkyl, or C₍₁₋₃₎-fluoroalkyl. In some cases, the fluoroalkyl is CF₃.

In some embodiments, the compound of formula (IVi-1) is of formula (X^{D}):

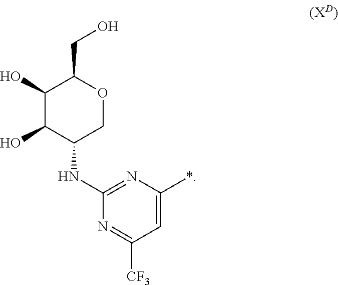

In some embodiments, the compound of formula (IVk-1) is of formula (X^{E}):

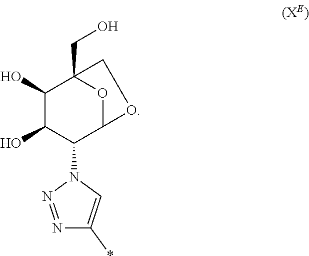 [SMILES: *c1cn([C@H]2C3OC[C@](CO)(O3)[C@H](O)[C@@H]2O)nn1]

In certain embodiments, the compound of formula (IVl) is of formula (IVl-1):

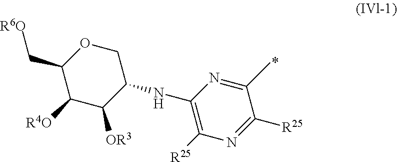

wherein:
R⁶, R⁴, R³, and R²¹ are as defined herein:
Y¹-Y⁴ are each independently N or CR²⁵;
Y⁵ is S, O, or NH; and
each R²⁵ is independently selected from H, optionally substituted C₍₁₋₆₎-alkyl, optionally substituted fluoroalkyl, and halogen.

In certain embodiments, each R²⁵ is H.

In certain embodiments, the ASGPR binding moiety (X) of the compounds of this disclosure can be described by one of the following structures:

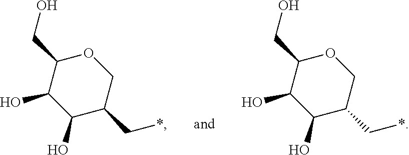

In certain embodiments, the ASGPR binding moiety (X) of the compounds of this disclosure can be described by one of the following structures:

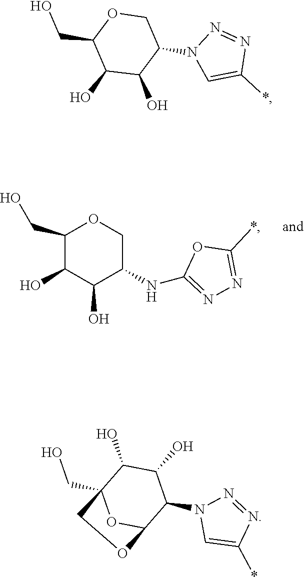

In certain embodiments, the ASGPR binding moiety (X) of the compounds of this disclosure can be described by one of the following structures:

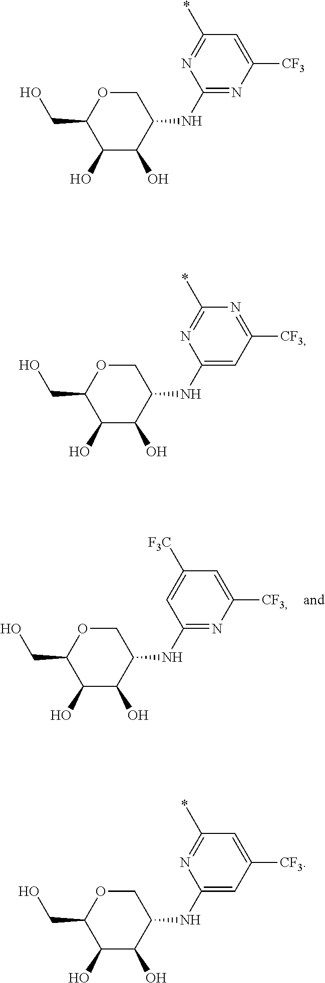 [SMILES: *c1cc(C(F)(F)F)nc(N[C@H]2CO[C@H](CO)[C@H](O)[C@@H]2O)n1 ; *c1nc(N[C@H]2CO[C@H](CO)[C@H](O)[C@@H]2O)cc(C(F)(F)F)n1 ; OC[C@H]1OC[C@H](Nc2cc(C(F)(F)F)cc(C(F)(F)F)n2)[C@@H](O)[C@H]1O ; *c1cc(C(F)(F)F)cc(N[C@H]2CO[C@H](CO)[C@H](O)[C@@H]2O)n1]

In certain embodiments of formula (IIb), R¹ R³, R⁴, and R¹¹ are H, and R⁶ is OH.

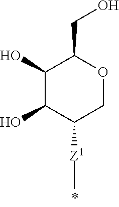

wherein Z¹ is —NH—, —CH₂—, —S— or —O—.

In certain embodiments of formula (IIb′), R³, R⁴ are H. and R⁶ is OH:

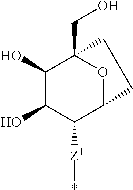

wherein Z¹ is —NH—, —CH₂—, —S—, —O—, triazole, e.g.

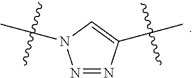 [SMILES: *c1cn(*)nn1]

6-linked ASGPR ligand moieties

In some embodiments, the ASGPR binding moiety (X) is linked via the 6-postion of the sugar analog. In some embodiments, the ASGPR binding moiety (X) has a reduced ring carbon at the 1-position relative to a galactosamine derived sugar.

In some embodiments, the ASGPR binding moiety (X) of the compounds of this disclosure can be described by formula (IIc):

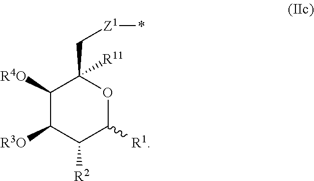

wherein R¹-R⁴ and Z¹ are as defined herein.

In certain embodiments of formula (IIc), Z¹ is selected from —O—, —S—, —CONR²¹—, and optionally substituted —(C(R²²)₂)_{q} heteroarylene, wherein q is 0 or 1. In certain cases, Z¹ is —O—. In certain other cases, Z¹ is optionally substituted —(C(R²²)₂)_{q}-triazole wherein q is 0 or 1. In certain cases, Z¹ is

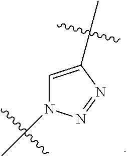 [SMILES: *c1cn(*)nn1]

In certain embodiments of formula (IIc), Z¹ is —Z¹¹-A¹-, wherein -A¹- is or optionally substituted -A¹- or optionally substituted arylene. In certain cases. -A¹- is an optionally substituted heteroarylene. In certain cases, the heteroarylene is a 5 or 6-membered heteroarylene. In certain cases, the heteroarylene is a 5-membered heteroarylene. In certain cases, the 5-membered heteroarylene is a triazole. In certain cases, the triazole is a 1,2,3-triazole moiety. In certain cases, Z¹¹ is —C(R²²)₂. In certain cases, at least one R²² is H. In certain cases, both R²² are H. In certain cases Z¹¹ is —O—. In certain cases, Z¹¹ is —S—. In certain other cases, Z¹¹ is —N(R²¹), where R²¹ is H or (C₁-C₃)alkyl. In certain cases, Z¹ is —C(R²²)₂-triazole-. In certain cases, Z¹ is:

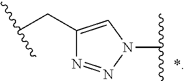 [SMILES: *Cc1cn(*)nn1]

In certain embodiments of formula (IIc), Z¹ is Z¹¹. In certain cases, Z¹¹ is —C(R²²)₂. In certain cases, at least one R²² is H. In certain cases, both R²² are H, and Z¹¹ is —CH₂—. In certain cases Z¹¹ is —O—. In certain cases, Z¹¹ is —S—. In certain other cases, Z¹¹ is —N(R²¹), where R²¹ is H or (C₁-C₃)alkyl.

In certain embodiments of formula (IIc), Z¹ is monocyclic 5 or 6-membered heteroarylene or arylene. In certain cases, Z¹ is

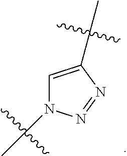 [SMILES: *c1cn(*)nn1]

In certain embodiments of formula (IIc), Z¹ is selected from —O—, —S—, —C(R²²)₂—, —N(R²¹)—CON(R²¹)—, and

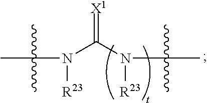

wherein:
X¹ is O or S;
t is 0 or 1;
R²¹ and each R²³ is independently selected from H, and optionally substituted (C₁-C₆)alkyl (e.g., C₍₁₋₃₎-alkyl, such as methyl); and
each R²² is independently selected from H, halogen (e.g., F) and optionally substituted (C₁-C₆)alkyl.

In certain embodiments, the compound of formula (IIc) is the following structure:

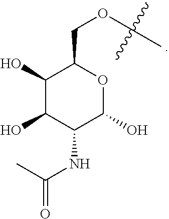 [SMILES: *OC[C@H]1O[C@H](O)[C@H](NC(C)=O)[C@@H](O)[C@H]1O]

In certain embodiments, the compound of formula (IIc) is the following structure:

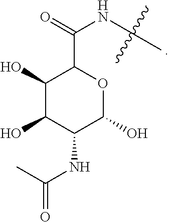 [SMILES: *NC(=O)C1O[C@H](O)[C@H](NC(C)=O)[C@@H](O)[C@H]1O]

In certain embodiments of formula (IIc), R¹¹ is H and the compound is of Table 5:

TABLE 5
Exemplary ASGPR binding moieties (X) of formula (IIc)

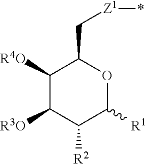

| # | R¹ | R² | R⁴ | R³ | Z¹ |
|---|---|---|---|---|---|
| X6 | —OH | —NHC(O)CH₃ | H | H | —O— |
| X7 | —OCH₃ | —NHC(O)CH₃ | H | H | —C(O)NH— |
| X8 | —OCH₃ | —NHC(O)CH₃ | H | H | —O— |
| | H | —NHC(O)CH₃ | H | H | —NH(O)C— |
| | nPr | —NHC(O)CH₃ | H | H | —NH(O)C— |

In some embodiments, the ASGPR binding moiety (X) of the compounds of this disclosure can be described by formula (IId):

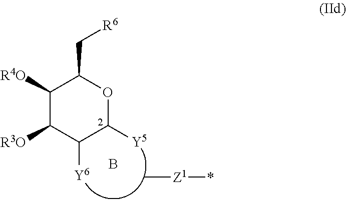

wherein:
R⁶, R⁴, R³ and Z¹ are as defined herein;
Y⁶ and Y⁵ are each independently selected from —O—, —S—, NR²¹—, and —C(R²²)₂;
R²¹ is selected from H, optionally substituted (C₁-C₆)alkyl, and —C(O)R²²;
each R²² is independently selected from H, halogen and optionally substituted (C₁-C₆)alkyl; and
ring B is a 5 or 6-membered optionally substituted cyclic group. In some embodiments of formula (IId), Y⁵ is connected to the sugar ring via an alpha configuration. In some embodiments of formula (IId), Y is connected to the sugar ring via a beta configuration.

In some embodiments, the ASGPR binding moiety (X) of the compounds of this disclosure can be described by formula (IId′):

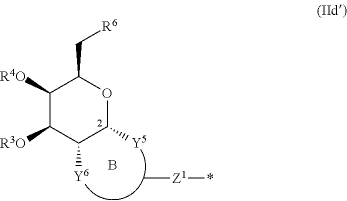

wherein:
R⁶, R⁴, R³ and Z¹ are as defined herein;
Y⁵ and Y⁶ are each independently selected from —O—, —S—, NR²¹—, and —C(R²²)₂;
R²³ is selected from H, optionally substituted (C₁-C₆)alkyl, and —C(O)R²²;
each R²² is independently selected from H, halogen and optionally substituted (C₁-C₆)alkyl; and
ring B is a 5 or 6-membered optionally substituted cyclic group.

In some embodiments of formula (IId)-(IId′) Y⁵ is O. In certain cases, Y⁵ is S. In certain cases. Y⁵ is —NR²¹—. In certain cases, Y⁵ is —C(R²²)₂ and each R²² is H.

In some embodiments of formula (IId)-(IId′) Y⁶ is —NR²¹— where R²¹ is H. In certain cases, Y⁶ is —NR²¹— where R²¹ is —C(O)R²². In some cases, R²² is methyl.

In some embodiments of formula (IId)-(IId′) the B ring is a 5 or 6-membered heterocycle. In some cases, the B ring is a 5-membered heterocycle. In some cases, the B ring is a 6-membered heterocycle.

In some embodiments of formula (IId)-(IId′) Z¹ is Z¹¹, where Z¹¹ is selected from —O—, —S—, NR²¹—, and —C(R²²)₂. In some cases, Z¹ is —O—. In some cases, Z¹ is —S—. In some cases, Z¹ is NR²¹ where R²¹ is H. In some cases, Z¹ is —C(R²²) where each R²² is H.

In some embodiments of formula (IId)-(IId′) Z¹ is optionally substituted Z¹¹-heteroarylene or optionally substituted Z¹¹-arylene. In some embodiments, Z¹ is CH₂-heteroarylene or CH₂-arylene. In some embodiments of formula (IId)-(IId′) Z¹ is optionally substituted amide. In some embodiments of formula (IId)-(IId′) Z¹ is optionally substituted sulfonamide. In some embodiments of formula (IId)-(IId′) Z is optionally substituted urea or optionally substituted thiourea.

In some embodiments, the compound of formula (IId)-(IId′) has one of the following structures:

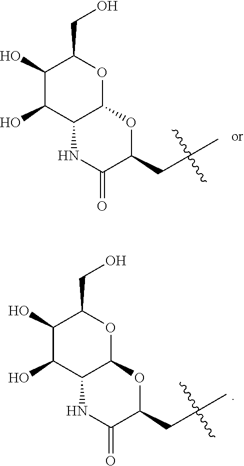 [SMILES: *C[C@@H]1O[C@H]2O[C@H](CO)[C@H](O)[C@H](O)[C@H]2NC1=O ; *C[C@@H]1O[C@@H]2O[C@H](CO)[C@H](O)[C@H](O)[C@H]2NC1=O]

In certain embodiments of any one of formulae (IIa), (IIb) or (IId), R⁶ is OH. In certain other cases. R⁶ is —OC(O)R. In certain cases, R⁶ is —C(O)NHR, where R is an optionally substituted alkyl. In certain cases, R terminates in an alkenyl or an alkynyl group. In certain other cases R⁶ is optionally substituted triazole. In certain cases, the triazole is of the following structure:

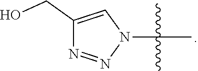 [SMILES: *n1cc(CO)nn1]

In certain embodiments of (Ila), and (IIc), R² is —NHCOCH₃. In certain other embodiments, R² is —NHCOCF₃. In certain other embodiments, R² is —NHCOCH₂CF₃. In certain cases, R² is —OH. In certain other cases. R² is an optionally substituted triazole. In certain cases, the triazole in of the following structure:

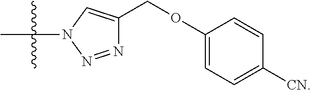 [SMILES: *n1cc(COc2ccc(C#N)cc2)nn1]

In certain embodiments when R⁶ or R² is a substituted triazole, the triazole is a 1,2,3-trizole, and the substituent is at the 4 or 5-position. In certain cases, the substituent on the triazole moiety includes but is not limited to, an optionally substituted (C₁₋₆)alkyl, optionally substituted (C₁₋₆)alkoxy, optionally substituted aryl, optionally substituted heteroaryl, optionally substituted alkaryl, and an optionally substituted alkyheteroaryl. It will be understood that any convenient substituent can be included in the triazole moiety, see, e.g., triazole moieties disclosed in Mamidayala et al, J. Am. Chem. Soc. 2012, 134, 1978-1981.

It is understood that the Z¹, Z¹¹, and Z¹¹—Ar linking moieties can be considered part of the X group of formula (I). In the ASGPR binding moieties (X) as described herein, —Z¹— can be linked to an -L¹- moiety (e.g., of the linker as described herein) via a variety of bonds and linking moieties, depending on the method of preparation. In some embodiments, the subject compounds comprise a —Z¹-L¹- moiety selected from:

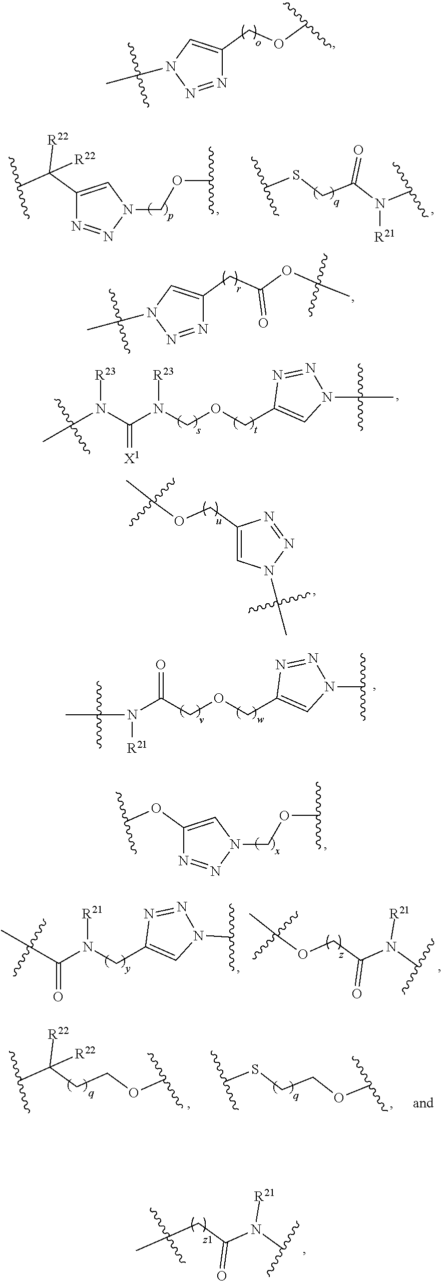

wherein each R²¹ is independently selected from H, and optionally substituted (C₁-C₆)alkyl; each R²² is independently selected from H, halogen (e.g., F) and optionally substituted (C₁-C₆)alkyl; and o, p, q, r, s, t, u, v, w, x, y, z and z1 are each independently 1 to 6. In certain embodiments, o, p, q, r, s, t, u, v, w, x, y, z and z1 are each independently 1 to 6.

In certain embodiments, the Z¹-L¹- group is

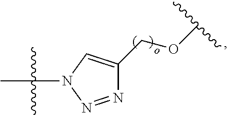

and o is 1 or 2.

In certain embodiments, the Z¹-L¹- group is

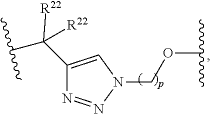

each R²² is H, and p is 1 or 2.

In certain embodiments, the Z¹-L¹- group is

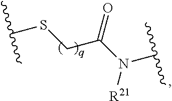

where q is 1-3.

In certain embodiments, the Z¹-L¹- group is

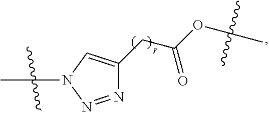

where r is 1-3.

In certain embodiments, the Z¹-L¹- group is

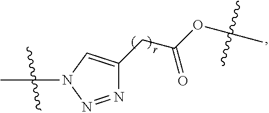

where r is 1-3.

In certain embodiments, the Z¹-L¹- group is

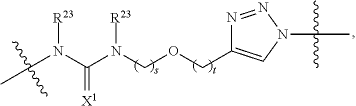

where s and t are each independently 1-3.

In certain embodiments, the Z¹-L¹- group is

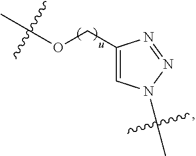

where u is 1-3.

In certain embodiments, the Z¹-L¹- group is

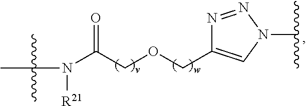

where v and w are each independently is 1-3.

In certain embodiments, the Z¹-L¹- group is

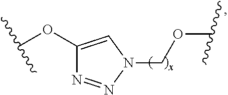

where x is 0-3.

In certain embodiments, the Z¹-L¹- group is

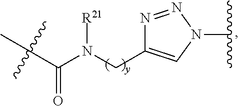

where y is 1-3.

In certain embodiments, the Z¹-L¹- group is

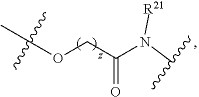

where R²¹ is H, and z is 1-4.

In certain embodiments, the Z¹-L¹- group is

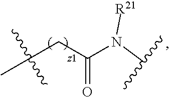

where R²¹ is H, and z1 is 1-4.

In certain embodiments, the Z¹-L¹- group is

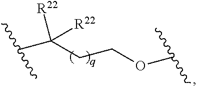

where each R²² is H, and q is 0-3. In certain embodiments, the Z¹-L¹- group is

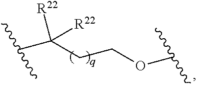

where each R²² is H, and q is 1-3.

In certain embodiments, the Z¹-L¹- group is

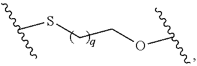

where q is 1-3.

In certain embodiments, the subject compounds comprise a —Z¹-L- group selected from:

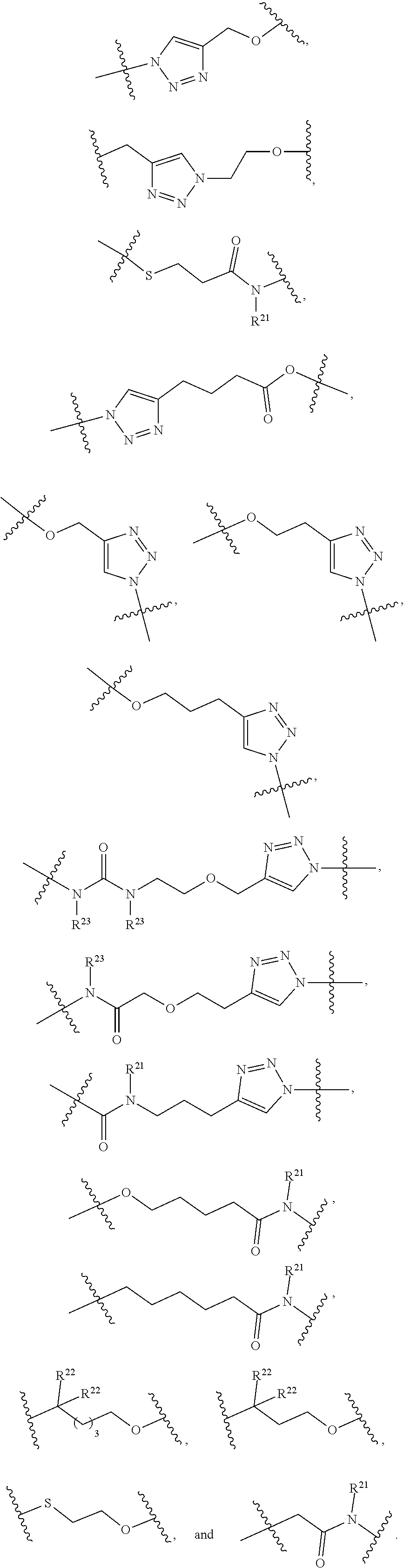

In certain embodiments, the Z¹-L¹- group is

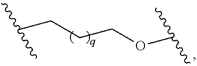

where q is 1-3. In certain cases, q is 1. In certain cases, q is 2. In certain cases, q is 3.

In certain embodiments, the Z¹-L¹- group is

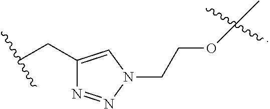 [SMILES: *Cc1cn(CCO*)nn1]

In certain embodiments, the Z¹-L¹- group is

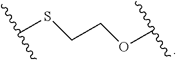 [SMILES: *OCCS*]

In certain embodiments, the Z¹-L¹- group is

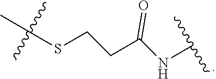 [SMILES: *NC(=O)CCS*]

In certain embodiments, —Z¹-L¹- comprises an optionally substituted —NH-heteroarylene-. In certain embodiments the heteroarylene is a triazole. In certain cases, the heteroarylene is pyridine. In certain cases, the heteroarylene is pyrimidine. In certain cases, the heteroarylene is thiadiazole.

In certain embodiments, the —Z¹-L¹- comprises a group selected from:

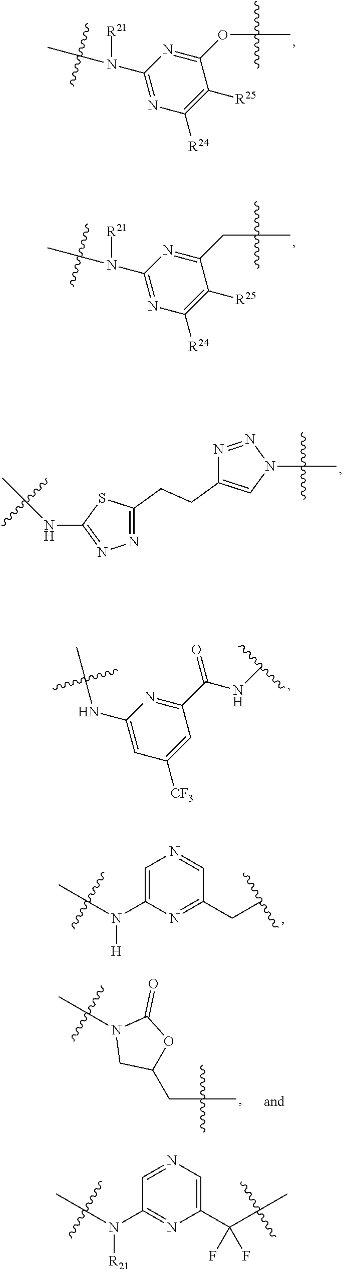

wherein each R²¹ is independently selected from H, optionally substituted (C₁-C₆)alkyl, and optionally substituted acyl; and R²⁴ and R²⁵ are each independently selected from H, optionally substituted C₍₁₋₆₎-alkyl, optionally substituted fluoroalkyl, and halogen.

In certain embodiments, the —Z¹-L¹- comprises a group selected from:

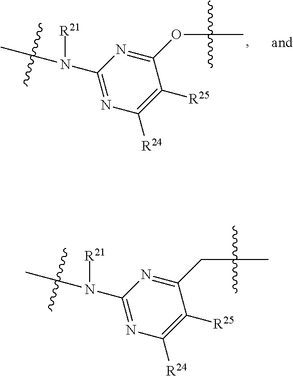

wherein R²⁴ and R²⁵ are each independently selected from H, optionally substituted C₍₁₋₆₎-alkyl, optionally substituted fluoroalkyl, and halogen; and each R²¹ is independently selected from H, optionally substituted (C₁-C₆)alkyl, and optionally substituted acyl. In certain cases, R²⁷ is H. In certain cases, R²⁴ is C₍₁₋₃₎-alkyl, or C₍₁₋₃₎-fluoroalkyl. In some cases, the fluoroalkyl is CF₃. In certain cases, R²⁵ is C₍₁₋₃₎-alkyl, or C₍₁₋₃₎-fluoroalkyl. In some cases, the fluoroalkyl is CF₃.

In certain embodiments, —Z¹-L¹- is

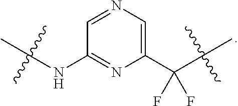 [SMILES: *Nc1cncc(C(*)(F)F)n1]

It is understood that a variety of substituents and chemistries can be utilized to connect a particular X ligand moiety (e.g., as described herein) to an adjacent linker. In some embodiments, a linking moiety of the linker comprises a triazole that derives from a Click chemistry conjugation. In certain embodiments, the ASGPR ligand moiety (X) is attached to a linking moiety as shown in one of the following structures:

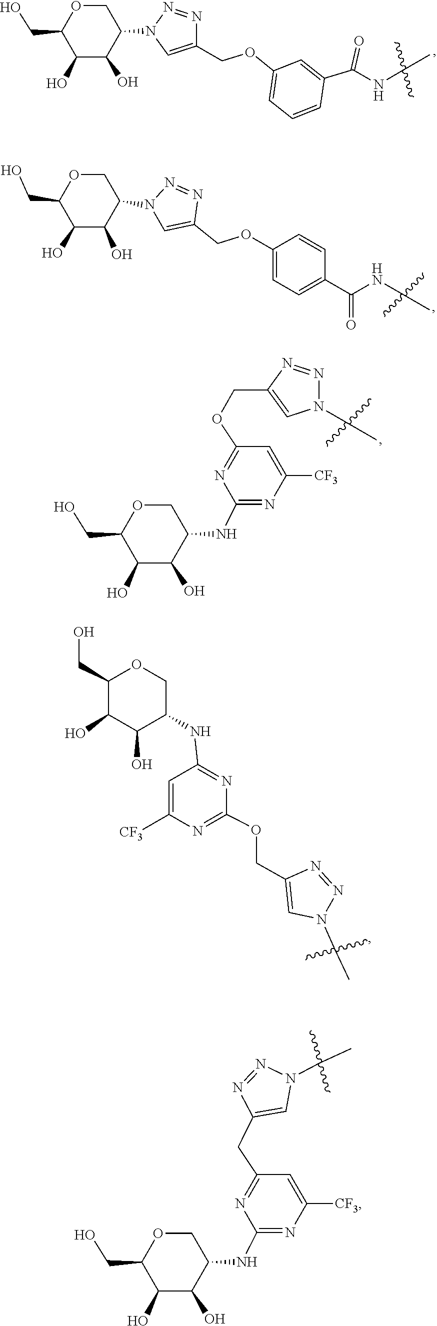 [SMILES: *NC(=O)c1cccc(OCc2cn([C@H]3CO[C@H](CO)[C@H](O)[C@@H]3O)nn2)c1 ; *NC(=O)c1ccc(OCc2cn([C@H]3CO[C@H](CO)[C@H](O)[C@@H]3O)nn2)cc1 ; *n1cc(COc2cc(C(F)(F)F)nc(N[C@H]3CO[C@H](CO)[C@H](O)[C@@H]3O)n2)nn1 ; *n1cc(COc2nc(N[C@H]3CO[C@H](CO)[C@H](O)[C@@H]3O)cc(C(F)(F)F)n2)nn1 ; *n1cc(Cc2cc(C(F)(F)F)nc(N[C@H]3CO[C@H](CO)[C@H](O)[C@@H]3O)n2)nn1]

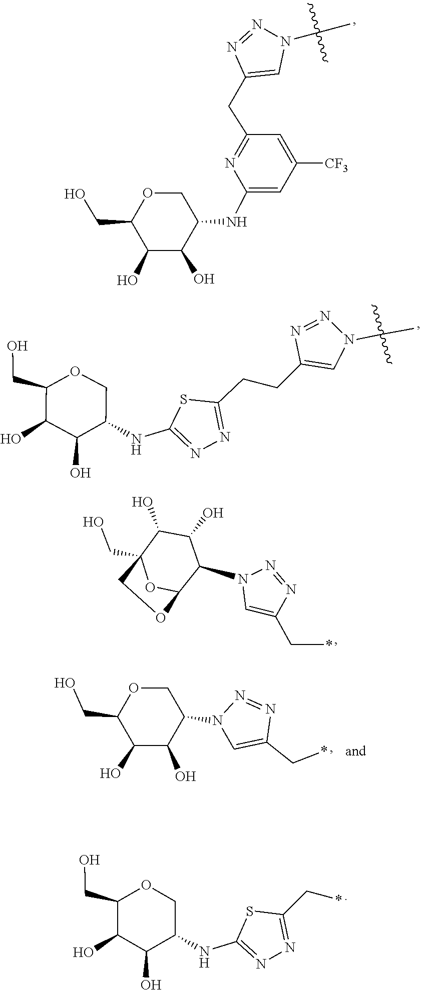

In certain embodiments of formula (IIb), R¹ R³, R⁴, and R¹¹ are H, and R⁶ is OH:

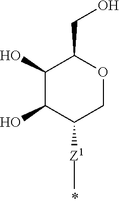

wherein Z¹ is triazole, —NH-heteroaryl (e.g., —NH— attached to pyridine or pyrimidine), —NH—, —O—, or —CH₂—, and/or Z¹ is attached to a linking moiety as shown in one of the following structures:

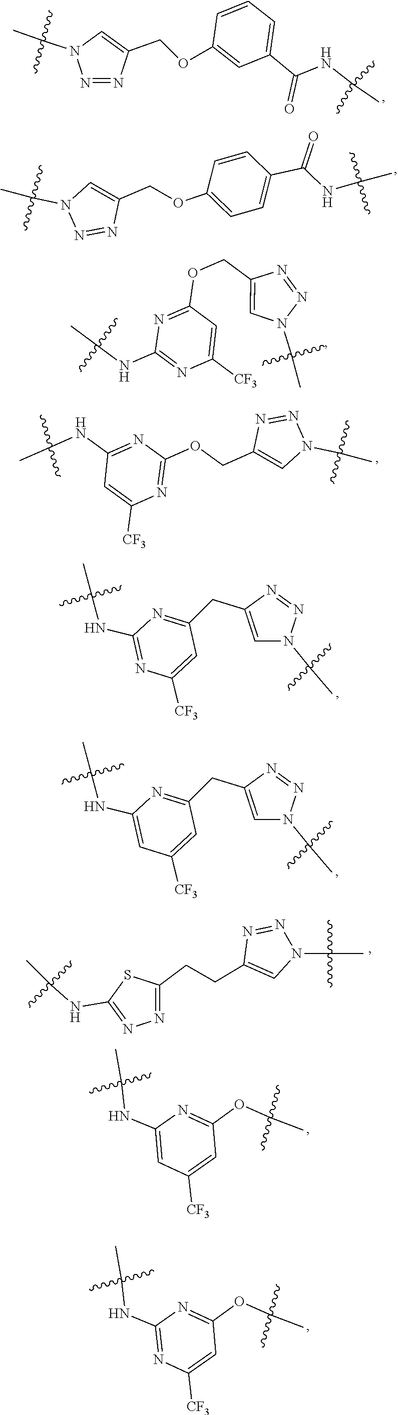

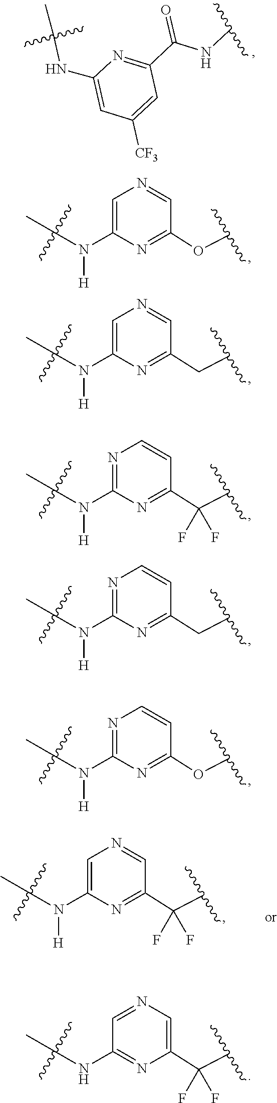

In certain embodiments, of formula (Iib), R¹ R³, R⁴, and R¹¹ are H, and R⁶ is OH:

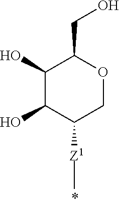

wherein Z¹ is attached to a linking moiety as shown in one of the following structures

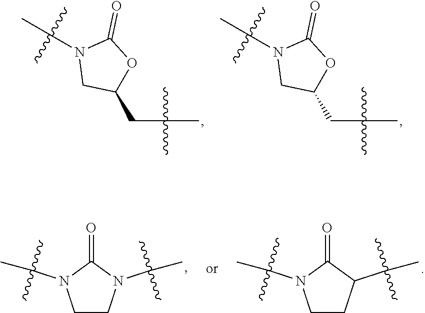 [SMILES: *C[C@H]1CN(*)C(=O)O1 ; *C[C@@H]1CN(*)C(=O)O1 ; *N1CCN(*)C1=O ; *C1CCN(*)C1=O]

### M6PR Binding Moiety

As summarized above, the M6PR binding moieties (also referred to as M6PR ligand moieties) of this disclosure can be linked to a variety of moieties of interest without impacting the specific binding to, and function of, the cell surface M6PR. The inventors have demonstrated that M6PR binding moieties having particular structures described below provide for high affinity binding to cell surface M6PRs, and when configured via a linker according to the bifunctional compounds of this disclosure can utilize the functions of cell surface M6PRs in a biological system, e.g., for internalization, and/or degradation of a target molecule such as an autoantibody.

The terms “mannose-6-phosphate receptor” and “M6PR” refer to receptors of the family of mannose-6-phosphate receptors. M6PRs are transmembrane glycoprotein receptors that target enzymes to lysosomes in cells. MP6R endogenously transports proteins bearing N-glycans capped with mannose-6-phosphate (M6P) residues to lysosomes, and cycles between endosomes, the cell surface, and the Golgi complex. See, e.g., Ghosh et al., Nat. Rev. Mol. Cell Biol. 2003; 4: 202-213. The family of M6PRs includes the cation independent mannose-6-phosphate receptor (CI-M6PR). The CI-M6PR is also referred to as the insulin-like growth factor 2 receptor (IGF2R) and is encoded in humans by the IGF2R gene (see, e.g., NCBI Reference Sequence: NM_000876.3, and NCBI Gene ID: 3482). The CI-M6PR binds insulin-like growth factor 2 (IGF-2) and mannose-6-phosphate (M6P)-tagged proteins. The compounds of this disclosure can specifically bind to a cell surface M6PR, for example, an internalizing CI-M6PR cell surface receptor. In particular embodiments, the surface CI-M6PR is a human CI-M6PR. It is understood that the terms M6PR and CI-M6PR are used interchangeably when referring to the binding properties of the M6PR binding moieties and compounds of this disclosure.

A compound comprising such M6PR binding moiety (X) (e.g., as described herein), may bind to other receptors, for example, may bind with lower affinity as determined by, e.g., immunoassays or other assays known in the art. In a specific embodiment, X, or a compound as described herein including such X specifically binds to a cell surface CI-M6PR with an affinity that is at least 2 logs. 2.5 logs, 3 logs, 4 logs or greater than the affinity when X or the compound bind to another cell surface receptor. In a specific embodiment, X, or a compound as described herein comprising X, specifically binds to CI-M6PR with an affinity (K_{d}) 20 mM or less. In particular embodiments, such binding is with an affinity (K_{d}) is 10 mM or less, 1 mM or less, 100 uM or less, 10 uM or less, 1 uM or less, 100 nM or less, 10 nM or less, or 1 nM or less. The terms “binds,” “binds to,” “specifically binds” or “specifically binds to” in this context are used interchangeably.

The M6PR binding compounds of this disclosure include a moiety (X) (e.g., as described herein) which is a D-mannopyranose analog that specifically binds to the cell surface receptor M6PR. The M6PR binding compounds can be monovalent or multivalent (e.g., bivalent or trivalent or of higher valency), where a monovalent compound includes a single M6PR ligand moiety, and a monovalent compound includes two or more such moieties.

### Alpha-Linked Pyranose Ring

The M6PR binding moiety of the compounds of this disclosure can include a linked pyranose ring described by formula (II):

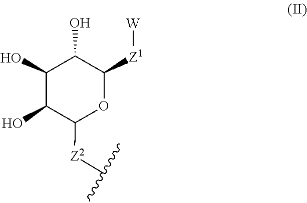

where:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

In some embodiments of formula (II), Z² is a linking moiety connected to the pyranose sugar ring at the anomeric or 1-position with an alpha-configuration as shown in formula (IIa) below:

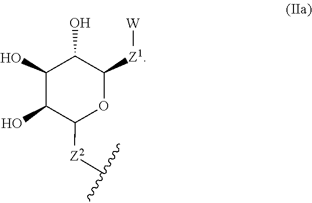

### Beta-Linked Pyranose Ring

The inventors have demonstrated that although M6PR binding compounds having a M6PR binding moiety with an anomeric alpha-configuration of formula (IIa) can provide good binding and internalization activity at the receptor, in some cases it is possible to impart more potent binding and internalization activity at the M6PR by configuring the central pyranose sugar ring of the M6PR binding moiety with a beta-configuration at the anomeric position. In some embodiments, such M6PR binding compounds can provide for increased stability at the pyranose ring.

Accordingly, in some embodiments of formula (II), Z² is a linking moiety connected to the sugar ring at the anomeric or 1-position with a beta-configuration as shown in formula (IIb) below:

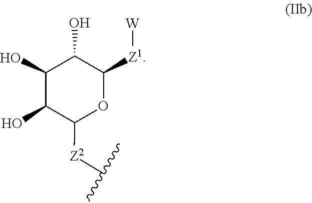

### M6PR Binding Compounds

Although moieties of formula (II) can exhibit binding activity for the M6PR, the inventors have demonstrated that when particular types of cyclic groups are linked with a particular configuration adjacent to the pyranose ring of formula (II) via the linking moiety Z², a M6PR binding moiety of desirable binding activity can be produced.

Accordingly, in some embodiments of formula (II), the M6PR binding moiety (X) can be described by formula (III):

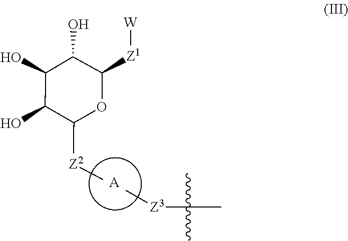

or a prodrug thereof, or a salt thereof, wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
A is independently an optionally substituted cyclic group; and
Z³ is independently a linking moiety.

In some embodiments of formula (II)-(III), W is a non-hydrolyzable hydrophilic head group.

In some embodiments of formula (II)-(III), Z² is optionally substituted ethylene. In some embodiments of formula (II)-(III), Z² is optionally substituted ethenylene.

In some embodiments of formula (II)-(III), Z² is O. In some embodiments of formula (II)-(III), Z² is S. In some embodiments of formula (II)-(III), Z² is —NR²¹—. In some embodiments of formula (II)-(III), Z² is —C(R²²)₂—, wherein each R²² is independently selected from H, halogen (e.g., F) and optionally substituted (C₁-C₆)alkyl. In some embodiments of formula (II)-(III), Z² is —CH₂—.

In some embodiments of formula (II)-(III), A is optionally substituted aryl, optionally substituted heteroaryl, optionally substituted heterocycle, or optionally substituted cycloalkyl. In some embodiments of formula (II)-(III), A is independently an optionally substituted aryl or heteroaryl linking moiety (e.g., monocyclic or bicyclic aryl or heteroaryl, optionally substituted).

Exemplary Z³ linking moieties of formula (II)-(III) are described herein.

Such M6PR-binding moieties of formula (III) can be attached to a moiety or molecule of interest to produce a bifunctional compound that undergoes effective M6PR-mediated cell internalization. The inventors have further demonstrated that when the moiety or molecule of interest is a target protein-binding moiety, the M6PR binding compound also provides for M6PR mediated internalization and/or degradation of bound target protein.

Accordingly, in some embodiments of formula (XI), the M6PR binding compound is of formula (XII):

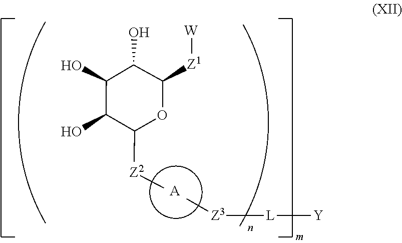

or a prodrug thereof, or a salt thereof,
wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²¹ is independently selected from H, halogen (e.g., F) and optionally substituted (C₁-C₆)alkyl;
A is independently an optionally substituted cyclic group;
Z³ is independently a linking moiety;
n is 1 to 500;
L is a linker:
Y is a moiety of interest; and
m is 1 to 100.

In some embodiments of formula (XI)-(XII), m is 1, and the cell surface M6PR binding compound is of formula (XIII):

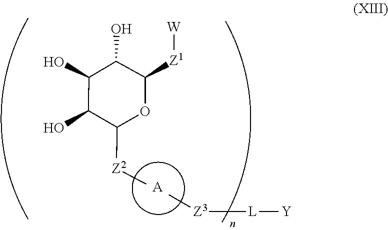

or a prodrug thereof, or a salt thereof,
wherein:
W is a hydrophilic head group;
Z¹ is selected from optionally substituted (C₁-C₃)alkylene and optionally substituted ethenylene;
Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl:
A is independently an optionally substituted cyclic group;
Z³ is independently a linking moiety;
n is 1 to 500;
L is a linker; and
Y is a moiety of interest (e.g., as described herein).

In some embodiments of formula (XIII). Y is a chemoselective ligation group. In some embodiments of formula (XIII), n is 1. In some embodiments of formula (XIII), Y is a chemoselective ligation group connected to “n” M6PR binding moieties (Xn-) via a single linker -L-. In some embodiments of formula (XIII), n is 2, 3, 4, or 5. In some embodiments of formula (XIII), n is 5-10. In some embodiments of formula (XIII), n is 10-100, such as 20-80, or 20-50. In some embodiments of formula (XIII), when n is 5 or more, then L is a polypeptide containing linker (e.g., as described herein).

In some embodiments of formula (XII)-(XIII), when n is 1 and A is phenyl, then: i) L comprises a backbone of at least 16 consecutive atoms (e.g., at least 18 consecutive atoms, or at least 20 consecutive atoms, in some cases up to about 200 consecutive atoms); ii) Y is a biomolecule; and/or ii) Z³ is amide, sulfonamide, urea or thiourea linking moiety to linker L.

In some embodiments of formula (XII), Z² is a linking moiety connected to the sugar ring at the anomeric or 1-position with an alpha-configuration as shown in formula (IIa) such that the compound is of formula (XIIa):

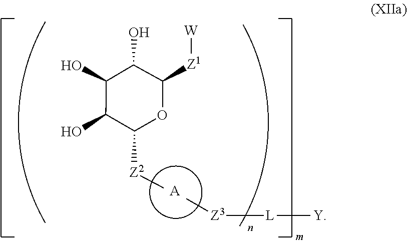

In some embodiments of formula (XII). Z² is a linking moiety connected to the sugar ring at the anomeric or 1-position with a beta-configuration as shown in formula (IIb), such that the compound is of formula (XIIb):

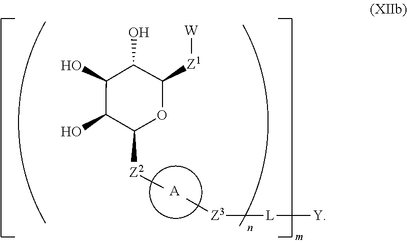

In some embodiments of formula (XI)-(XIIb), multiple M6PR binding moieties, e.g., of formula (III), are linked via multiple linkers L to different ligation sites on a moiety of interest Y. In some embodiments, when Y is a biomolecule, the compound of formula (XI)-(XIIb) can be referred to as a conjugate.

### Hydrophilic Head Group and Linking Moieties

In some embodiments of formula (II)-(XIII), the M6PR binding moiety (X) includes an analog of a D-mannopyranose ring, with a hydrophilic head group, or a precursor or prodrug thereof, that is connected via a linking moiety (Z¹) to the 5-position of the sugar ring. The linking moiety can be of 1-6 atoms in length, such as 1-5, 1-4 or 1-3 atoms in length, e.g., 1 or 2 atoms in length. It is understood that the length of the linking moiety can be selected in conjunction with the hydrophilic head group.

The hydrophilic head group (W) can be any suitable negatively charged group, or salt thereof. In some embodiments, the hydrophilic head group is a neutral, polar, hydrophilic group. In general, the hydrophilic head group is capable of hydrogen bonding or electrostatic interactions with the M6PR, under aqueous or physiological conditions, similar to those of the phosphate group of M6P. The hydrophilic head group can be a bioisostere (e.g., a structural or functional mimic) of the 6-phosphate group of the naturally occurring mannose-6-phosphate ligand. In some embodiments, the hydrophilic head group is non-hydrolyzable, i.e., a functional group that is stable against its cleavage (e.g., chemically or enzymatically) under physiological conditions, from the Z¹ linking moiety and/or pyranose ring of X to which the hydrophilic head group is attached.

The hydrophilic head group is generally a small group, such as a heteroatom containing functional group, or single heterocyclic ring, and in some cases has a MW of less than 200, such as less than 150, or less than 100.

In some embodiments, the hydrophilic head group is a phosphonate, or a bioisostere thereof, such as a carboxylate or malonate. In some embodiments, the hydrophilic head group is a thiophosphonate.

In some embodiments of formula (II)-(XIII), the hydrophilic head group is not a phosphate, thiophosphate or dithiophosphate, as such groups would have phosphate ester linkages to the compound which can be unstable and susceptible to cleavage under physiological conditions (e.g., by phosphatases in a biological system or chemically). For example, the 6-phosphate ester group of M6P exhibits undesirable stability as compared to a phosphonate analog, or other more stable head group. This disclosure provides alternative non-hydrolyzable head groups in addition to phosphonate which retain binding and internalization activity of the resulting M6PR binding compound.

In any one of the embodiments of formula (II)-(XIII), the hydrophilic head group W is selected from —OH, —CR²R²OH, —NR³P═O(OH)₂, —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

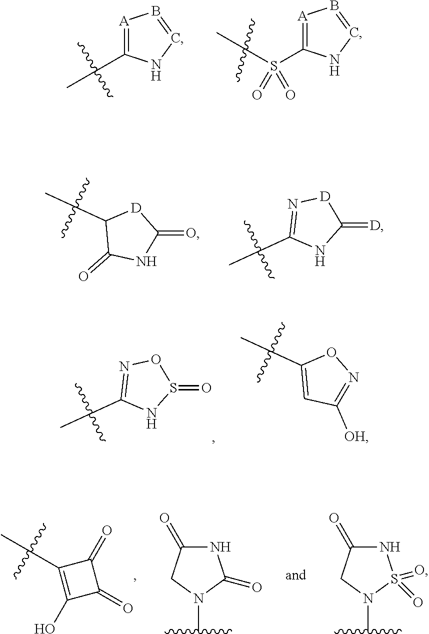

or a salt thereof,
wherein:
R¹ and R² are independently hydrogen, SR³, halo, or CN, and R³ and R⁴ are independently H, C₁₋₆alkyl or substituted C₁₋₆alkyl (e.g., —CF₃ or —CH₂CF₃);
A, B, and C are each independently CH or N; and
D is each independently O or S.

In some embodiments of formula (II)-(XIII), the hydrophilic head group W is phosphate or thiophosphate, e.g., —OP═O(OH)₂, —SP═O(OH)₂, —OP═O(SH)(OH), —SP═O(SH)(OH), —OP═S(OH)₂, —OP═O(N(R³)₂)(OH), or —OP═O(R³)(OH), or a salt thereof. In some embodiments of formula (II)-(XIII), the hydrophilic head group W is non-hydrolyzable, and accordingly, is not selected from phosphate or thiophosphate, e.g., —OP═O(OH)₂, —SP═O(OH)₂, —OP═O(SH)(OH), —SP═O(SH)(OH), —OP═S(OH)₂, —OP═O(N(R³)₂)(OH), or —OP═O(R³)(OH), or a salt thereof.

In some embodiments of formula (II)-(XIII), the hydrophilic head group W is charged. e.g., capable of forming a salt under aqueous or physiological conditions. In some embodiments of formula (II)-(XIII), the hydrophilic head group W is selected from —NR³P═O(OH)₂, —P═O(OH)₂, —P═S(OH)₂, —P═O(SH)(OH), —P═S(SH)(OH), P(═O)R¹OH, —PH(═O)OH, —(CR²R²)—P═O(OH)₂, —COOH, —CH(COOH)₂, —CR¹R²COOH, and —NHC(O)CO₂H.

In some embodiments of formula (JJ)-(XIII), the hydrophilic head group W is phosphonate or thiophosphonate (e.g., —P═O(OH)₂, —P═S(OH)₂, —P═O(SH)(OH), or —P═S(SH)(OH), or a salt thereof). In some embodiments of formula (II)-(XIII), the hydrophilic head group W is phosphonate or a salt thereof. In some embodiments of formula (II)-(XIII), the hydrophilic head group W is —CO₂H or a salt thereof. In some embodiments of formula (II)-(XIII), the hydrophilic head group W is malonate (e.g., —CH(COOH)₂ or a salt thereof).

In some embodiments of formula (II)-(XIII), the hydrophilic head group W is selected from —SOOH (i.e., —SO₃H), —S(O)OH, —OSO₂OH, and —NHSO₃H. In some embodiments of formula (II)-(XIII), the hydrophilic head group W is sulfonate (e.g., —SO₃H or a salt thereof).

In some embodiments, the hydrophilic head group W is neutral hydrophilic. In some embodiments of formula (II)-(XIII), the hydrophilic head group W is selected from —OH, —CR²R²OH, —CN, —CONH₂, —CONHR³, —CONR³R⁴, —CONH(OH), —CONH(OR³), —CONHSO₂R³, —SO₂R³, —SOR³R⁴, —SO₂NH₂, —SO₂NHR³, —SO₂NR³R⁴, —SO₂NHCOR³, —NHCOR³, —NHSO₂NHR³, —NHC(O)NHS(O)₂R³, and —NHSO₂R³.

In some embodiments of formula (II)-(XIII), the hydrophilic head group W comprises a heterocycle, such as

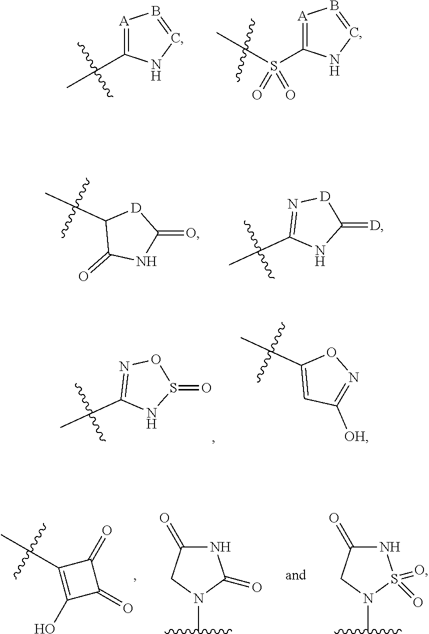

or a salt thereof,
wherein A, B, and C are each independently CH or N; and D is each independently O or S.

In some embodiments of formula (II)-(XIII), the hydrophilic head group W comprises a 5-membered heterocycle, such as

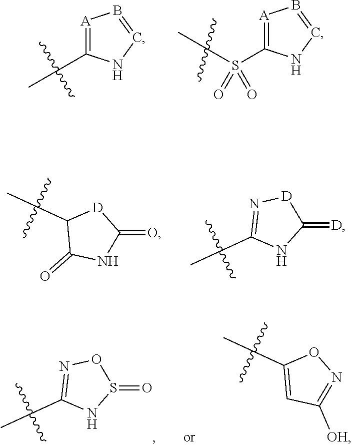

or a salt thereof.

In some embodiments of formula (II)-(XIII), the hydrophilic head group W is linked to the pyranose ring via a Z¹ that is selected from optionally substituted (C₁-C₂)alkylene and optionally substituted ethenylene. The Z¹ can be selected in conjunction with W so as to provide a desired spacing between the 5-position of the ring and the charged or polar center of W. For example, when W is a malonate having a CH atom linking the two carboxylic acid groups, Z¹ can be methylene, which together provide a desirable two carbon spacer between the ring and the COOH groups.

In some embodiments of formula (II)-(XIII), Z¹ is methylene or substituted methylene. In some embodiments of formula (II)-(XIII), Z¹ is ethyl or substituted ethyl. In some embodiments of formula (II)-(XIII), Z¹ is ethenylene or substituted ethenylene. In some embodiments of formula (II)-(XIII), Z¹ is substituted with one or more halogen, e.g., fluoro.

In some embodiments of formula (III), the M6PR binding moiety (X) is described by one of formula (IV-1) to (IV-3):

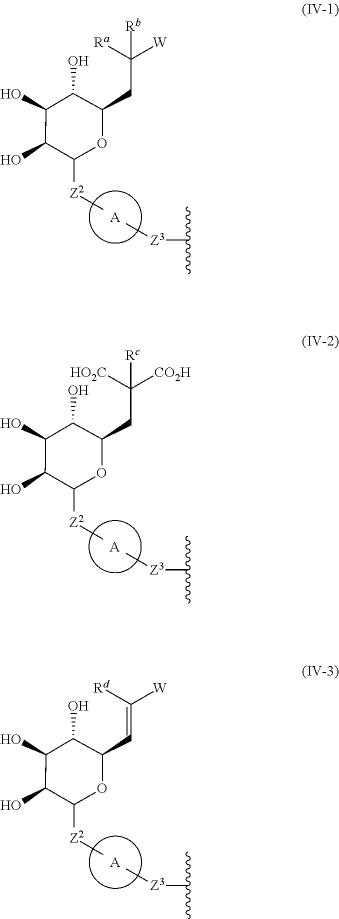

wherein R^{a}, R^{b}, R^{c} and R^{d} are independently H or F.

In some embodiments of formula (IV-1) to (IV-3), Z² is O.

In some embodiments of formula (IV-1) to (IV-3), Z² is S.

In some embodiments of formula (IV)-1 to (IV-3), Z² is —NR²¹—.

In some embodiments of formula (IV-1) to (IV-3), Z² is —C(R²²)₂—, wherein each R²² is independently selected from H, halogen (e.g., F) and optionally substituted (C₁-C₆)alkyl. In some embodiments of formula (IV-1) to (IV-3), Z² is —CH₂—.

In some embodiments of formula (IV-1) to (IV-3), R^{a}, R^{b}, R^{c} and R^{d} are each H.

In some embodiments of formula (IV-1) R^{a} is H and R^{b} is F. In some embodiments of formula (IV-1) R^{a} and R^{b} are each F.

In some embodiments of formula (IV-2) R^{c} is H. In some embodiments of formula (IV-2) R^{c} is F.

In some embodiments of formula (IV-3) R^{d} is H. In some embodiments of formula (IV-3) R^{d} is F.

In some embodiments of formula (IV-1) and (IV-3), W is selected from —P═O(OH)₂, —P═S(OH)₂, —P═O(SH)(OH), —P═S(SH)(OH), and —COOH, or a salt thereof. In some embodiments of formula (IV-1) and (IV-3). W is —P═O(OH)₂, or a salt thereof. In some embodiments of formula (IV-1) and (IV-3). W is COOH, or a salt thereof.

In some embodiments of formula (IV-1) R^{a} and R^{b} are each F, and W is —P═O(OH)₂, or a salt thereof. In some embodiments of formula (IV-1) R^{a} and R^{b} are each H, and W is —P═O(OH)₂, or a salt thereof. In some embodiments of formula (IV-1) R^{a} is F, R^{b} is H, and W is —P═O(OH)₂, or a salt thereof.

In some embodiments of formula (IV-1) to (IV-3), Z² is linked to the anomeric position of the pyranose ring with an alpha-configuration. In such cases, the M6PR binding moiety (X) of (IV-1) to (IV-3) can be referred to as formula (IV-A1) to (IV-A3), respectively.

In some embodiments of formula (IV-AT) to (IV-A3), Z² is S. In some embodiments of formula (IV-A1) to (IV-A3), Z² is O. In some embodiments of formula (IV-AT) to (IV-A3), Z² is —CH₂—. In some embodiments of formula (IV-A1) to (IV-A3), Z² is —CF₂—.

In some embodiments of formula (IV-A1) and (IV-A3), W is selected from —P═O(OH)₂, —P═S(OH)₂, —P═O(SH)(OH), —P═S(SH)(OH), and —COOH, or a salt thereof. In some embodiments of formula (IV-A1) and (IV-A3), W is —P═O(OH)₂, or a salt thereof. In some embodiments of formula (IV-A1) and (IV-A3), W is COOH, or a salt thereof.

In some embodiments of formula (IV-A1) R^{a} and R^{b} are each F, and W is —P═O(OH)₂, or a salt thereof. In some embodiments of formula (IV-A1) R^{a} and R^{b} are each H, and W is —P═O(OH)₂, or a salt thereof. In some embodiments of formula (IV-A1) R^{a} is F. R^{b} is H. and W is —P═O(OH)₂, or a salt thereof.

In some embodiments of formula (IV-1) to (IV-3), Z² is linked to the anomeric position of the pyranose ring with a beta-configuration. The inventors demonstrated that a compound including a M6PR binding moiety having a β-glycoside configuration can have at least equivalent binding and/or cellular uptake activity as compared to a conjugate having the corresponding α-glycoside configuration. In some embodiments, such M6PR binding moieties having a β-glycoside configuration can provide increased stability as compared to a reference compound having a β-glycoside configuration. Accordingly, in some embodiments of formula (IV), the M6PR binding moiety (X) is described by one of formula (IV-B1) to (IV-B3):

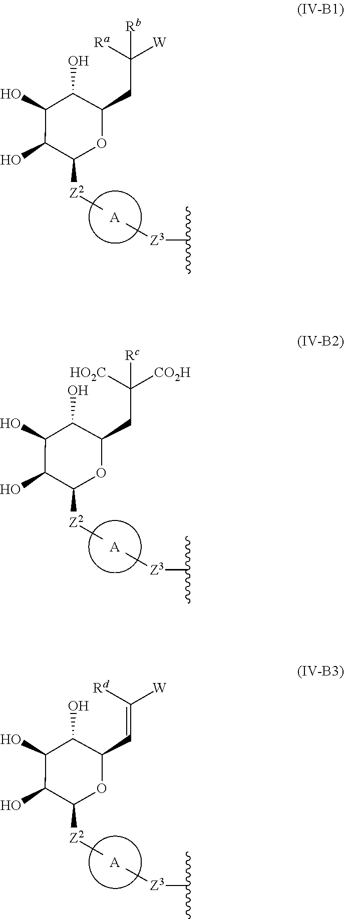

wherein R^{a}, R^{b}, R^{c} and R^{d} are independently H or F.

In some embodiments of formula (IV-B1) to (IV-B3), Z² is S. In some embodiments of formula (IV-B1) to (IV-B3), Z² is O. In some embodiments of formula (IV-B1) to (IV-B3), Z² is —CH₂—. In some embodiments of formula (IV-B1) to (IV-B3), Z² is —CF₂—.

In some embodiments of formula (IV-B1) and (IV-B3), W is selected from —P═O(OH)₂, —P═S(OH)₂, —P═O(SH)(OH), —P═S(SH)(OH), and —COOH, or a salt thereof.

In some embodiments of formula (IV-B1) and (IV-B3), W is selected from —P═O(OH)₂, —P═S(OH)₂, —P═O(SH)(OH), —P═S(SH)(OH), and —COOH, or a salt thereof. In some embodiments of formula (IV-B1) and (IV-B3). W is —P═O(OH)₂, or a salt thereof. In some embodiments of formula (IV-B1) and (IV-B3), W is COOH, or a salt thereof.

In some embodiments of formula (IV-B1) R^{a} and R^{b} are each F, and W is —P═O(OH)₂, or a salt thereof. In some embodiments of formula (IV-B1) R^{a} and R^{b} are each H, and W is —P═O(OH)₂, or a salt thereof. In some embodiments of formula (IV-B1) R^{a} is F, R^{b} is H, and W is —P═O(OH)₂, or a salt thereof.

The inventors demonstrated that a conjugate including M6PR binding moiety having a β-S-glycoside configuration can have at least equivalent or superior binding and/or cellular uptake activity as compared to a conjugate having the corresponding α-S-glycoside configuration, or to a conjugate having an α-O-glycoside configuration. See FIG. 19.

Accordingly, in some embodiments of formula (IV-B1) to (IV-B3), the M6PR binding moiety (X) is described by one of formula (IV-BS1) to (IV-BS3):

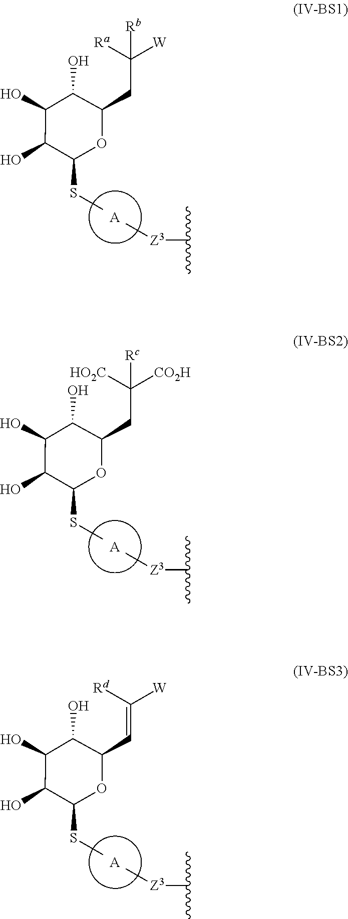

wherein R^{a}, R^{b}, R^{c} and R^{d} are independently H or F.

In some embodiments of formula (IV-BS1) to (IV-BS3), R^{a}, R^{b}, R^{c} and R^{d} are each H.

In some embodiments of formula (IV-BS1) R^{a} is H and R^{b} is F. In some embodiments of formula (IV-BS1) R^{a} and R^{b} are each F.

In some embodiments of formula (IV-BS2) R^{c} is H. In some embodiments of formula (IV-B2) R^{c} is F.

In some embodiments of formula (IV-BS3) R^{d} is H. In some embodiments of formula (IV-BS3) R^{d} is F.

In some embodiments of formula (IV-BS1) to (IV-BS3), Z² is S. In some embodiments of formula (IV-BS1) to (IV-BS3), Z² is O. In some embodiments of formula (IV-BS1) to (IV-BS3), Z² is —CH₂—. In some embodiments of formula (IV-BS1) to (IV-BS3), Z² is —CF₂—.

In some embodiments of formula (IV-BS1) and (IV-BS3), W is selected from —P═O(OH)₂, —P═S(OH)₂, —P═O(SH)(OH), —P═S(SH)(OH), and —COOH, or a salt thereof. In some embodiments of formula (IV-BS1) and (IV-BS3), W is —P═O(OH)₂, or a salt thereof. In some embodiments of formula (IV-BS1) and (IV-BS3), W is COOH, or a salt thereof.

In some embodiments of formula (IV-BS1) R^{a} and R^{b} are each F, and W is —P═O(OH)₂, or a salt thereof. In some embodiments of formula (IV-BS1) R^{a} and R^{b} are each H, and W is —P═O(OH)₂, or a salt thereof. In some embodiments of formula (IV-BS1) R^{a} is F, R^{b} is H, and W is —P═O(OH)₂, or a salt thereof.

In some embodiments, the mannose ring or analog thereof of the M6PR binding moiety can be incorporated into the compounds of this disclosure by attachment of a linking moiety to the Z² group attached at the anomeric or 1-position of the sugar ring.

In some embodiments, the M6PR binding moiety is incorporated into the compounds of this disclosure by attachment of a linker to the Z³ group attached to the cyclic group A. It is understood that in the compounds of formula (III), the cyclic group attached to Z² can be considered part of the M6PR binding moiety (X) and provide for a desirable binding property to the M6PR.

### Cyclic Group A

The A cyclic group of formula (III)-(XIII) can be a monocyclic or bicyclic group. A bicyclic group of interest can be a fused bicyclic group or a bicyclic group containing two monocyclic linked via a covalent bond. The A cyclic group of formula (III)-(XIII) can be optionally substituted aryl, optionally substituted heteroaryl, optionally substituted heterocycle (e.g., saturated heterocycle), or optionally substituted cycloalkyl.

The A cyclic group of formula (III)-(XIII) can be a monocyclic aryl or monocyclic heteroaryl group. In some embodiments of formula (III)-(XIII), A is a 5-membered monocyclic heteroaryl group. In some embodiments of formula (III)-(XIII), A is a 6-membered monocyclic aryl or heteroaryl group. In some embodiments of formula (III)-(XIII), A can be a multicyclic aryl or multicyclic heteroaryl group, such as a bicyclic aryl or bicyclic heteroaryl group. In some embodiments of formula (III)-(XIII). A is a fused bicyclic group. In some embodiments of formula (III)-(XIII), A is a bicyclic group comprising two aryl and/or heteroaryl monocyclic rings connected via a covalent bond. In some embodiments of formula (III)-(XIII), A is a bicyclic aryl or bicyclic heteroaryl group having two 6-membered rings. In some embodiments of formula (III)-(XIII), A is a bicyclic aryl or bicyclic heteroaryl group having one 6-membered ring that is connected via a covalent bond or fused to a 5-membered ring.

In some embodiments of formula (III)-(XIII), A is selected from optionally substituted phenyl, optionally substituted pyridyl, optionally substituted biphenyl, optionally substituted naphthalene, optionally substituted quinoline, optionally substituted triazole and optionally substituted phenylene-triazole.

In some embodiments of formula (III)-(XIII), A is not phenyl (also referred to as phenylene in the context of formula (III), e.g., 1,4-phenylene).

In some embodiments of formula (III)-(XIII), A is substituted with at least one OH substituent. In some embodiments of formula (III)-(XIII), A is substituted with 1, 2, or more OH groups. In some embodiments of formula (III)-(XIII), A is substituted with at least one optionally substituted (C₁-C₆)alkyl.

In some embodiments of formula (III)-(XIII), A is optionally substituted 1,4-phenylene, optionally substituted 1,3-phenylene, or optionally substituted 2,5-pyridylene.

In some embodiments of formula (III)-(XIII), A is selected from:

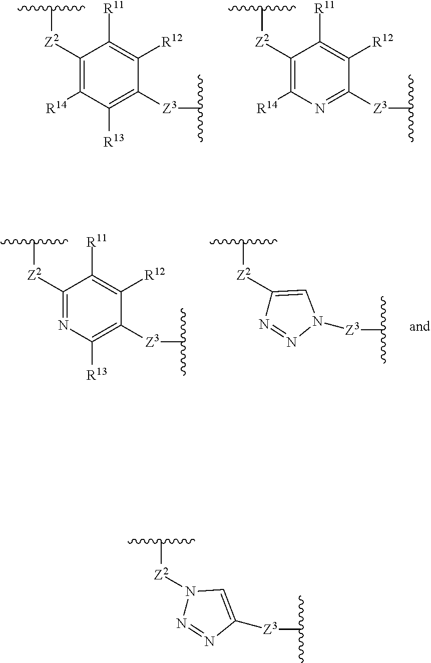

wherein:
R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
R²⁵ is independently selected from H, and optionally substituted (C₁-C₆)alkyl.

In some embodiments of formula (III)-(XIII), A is optionally substituted fused bicyclic aryl or optionally substituted fused bicyclic heteroaryl.

In some embodiments of formula (III)-(XIII), A is optionally substituted naphthalene or optionally substituted quinoline.

In some embodiments of formula (III)-(XIII), A is selected from:

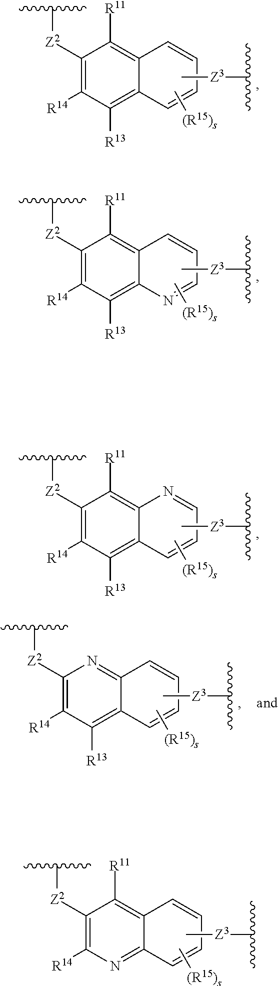

wherein:
R¹¹ and R¹³ to R¹⁴ is independently selected from H, halogen. OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

In some embodiments of formula (II)-(XIII), A is selected from:

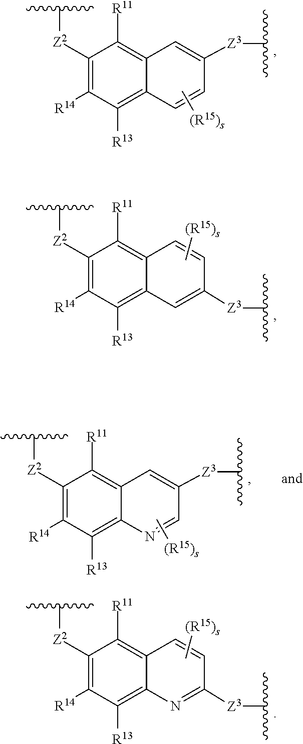

In some embodiments of formula (III)-(XIII), A is optionally substituted bicyclic aryl or optionally substituted bicyclic heteroaryl of following formula:

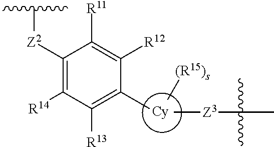

or a salt thereof, wherein:
Cy is independently monocyclic aryl or monocyclic heteroaryl;
R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 4; and
each R²¹ is independently selected from H, and optionally substituted (C₁-C₆)alkyl.

In some embodiments, when Cy is optionally substituted phenyl, then A is optionally substituted biphenyl of the formula:

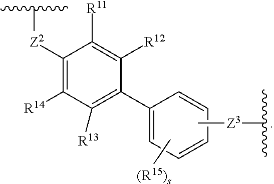

In some embodiments of formula (III)-(XIII), A is selected from:

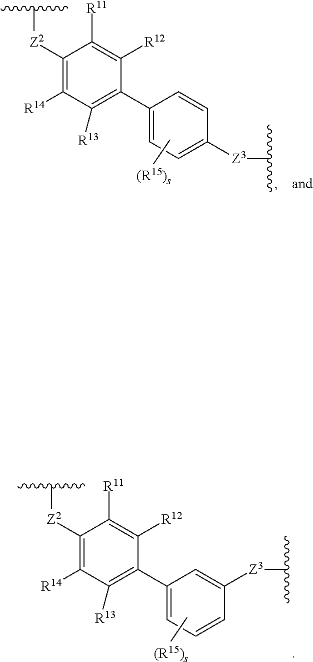

In some embodiments, when Cy is triazole, then A is selected from:

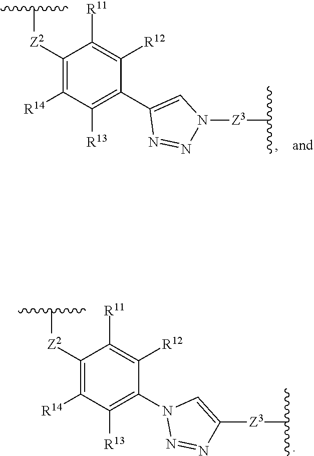

In some embodiments, at least one of R¹¹ to R¹⁵ is OH (e.g., at least two are OH).

In some embodiments, R¹¹ to R¹⁵ are each H.

### Linking Moiety Z³

The linking moiety Z³ can be any convenient linking moiety that connects the linker L to the cyclic ring A. In some embodiments of formula (III)-(XIII), Z³ is has a backbone of 3 atoms or less.

In some embodiments of formula (III)-(XIII), Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (III)-(XIII), Z³ is a covalent bond connecting A to L.

In some embodiments of formula (III)-(XIII), Z³ is optionally substituted amido, urea or thiourea.

In some embodiments of formula (III)-(XIII), Z³ is

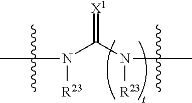

wherein:
X¹ is O or S;
t is 0 or 1; and
each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is urea or thiourea.

In some embodiments of formula (III)-(XIII), Z³ is —N(R²³)SO₂— or —SO₂N(R²³)—. In some embodiments of formula (III)-(XIII), Z³ is —NHSO₂— or —SO₂NH—.

In some embodiments of formula (III)-(XIII), Z³ is —N(R²³)CO— or —CON(R²³)—. In some embodiments of formula (III)-(XIII), Z³ is —NHCO— or —CONH—.

In some embodiments of formula (III)-(XIII), Z³ is —NHC(═X¹)NH—, wherein X¹ is O or S. In some embodiments. X¹ is O (i.e., Z³ is —NHC(═O)NH—). In some embodiments. X¹ is S.

In some embodiments of formula (III)-(XIII), Z³ is optionally substituted triazole. When Z³ is optionally substituted triazole, it can be synthetically derived from click chemistry conjugation of an azido containing precursor and an alkyne containing precursor of the compound.

In some embodiments, Z³ is selected in combination with cyclic group A and/or linking moiety Z to provide desirable M6PR binding and internalization properties for X.

In some embodiments of formula (III)-(XIII), -A-Z³— is selected from:

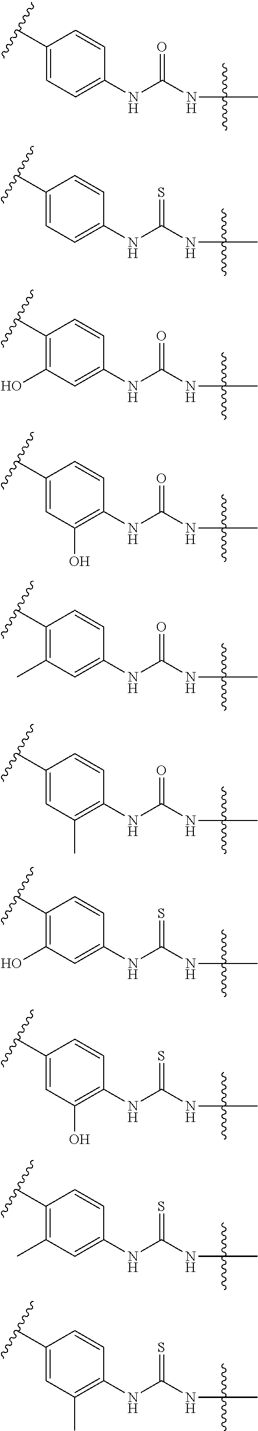

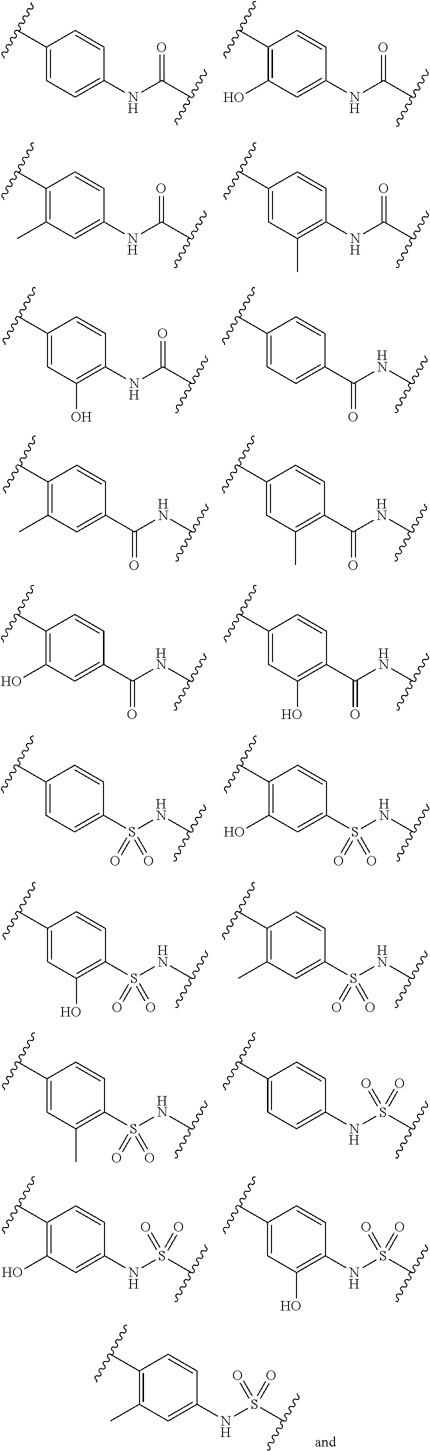

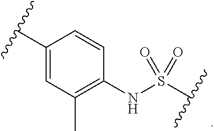 [SMILES: *c1ccc(NS(*)(=O)=O)c(C)c1]

In some embodiments of formula (III)-(XIII), -A-Z³— is selected from:

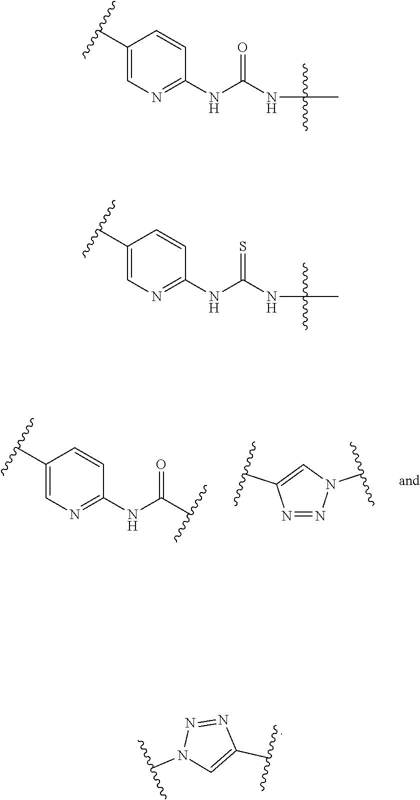 [SMILES: *NC(=O)Nc1ccc(*)cn1 ; *NC(=S)Nc1ccc(*)cn1 ; *C(=O)Nc1ccc(*)cn1 ; *c1cn(*)nn1 ; *c1cn(*)nn1]

In some embodiments of formula (III)-(XIII), -A-Z³— is selected from:

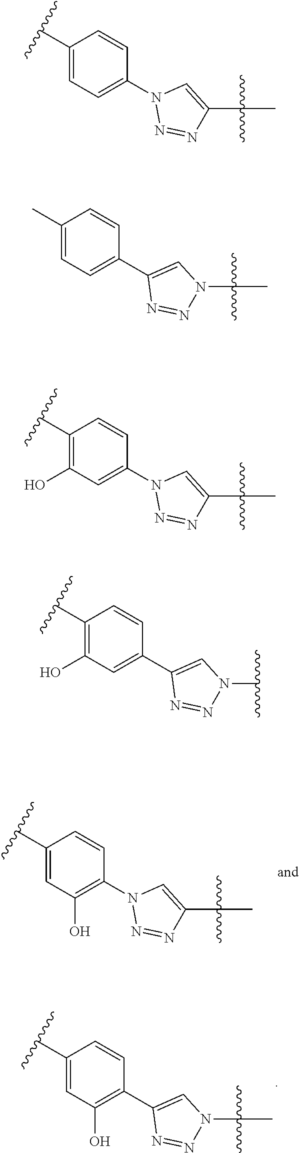 [SMILES: *c1ccc(-n2cc(*)nn2)cc1 ; *n1cc(-c2ccc(C)cc2)nn1 ; *c1cn(-c2ccc(*)c(O)c2)nn1 ; *c1ccc(-c2cn(*)nn2)cc1O ; *c1ccc(-n2cc(*)nn2)c(O)c1 ; *c1ccc(-c2cn(*)nn2)c(O)c1]

In some embodiments of formula (II)-(XIb), -A-Z³— is selected from:

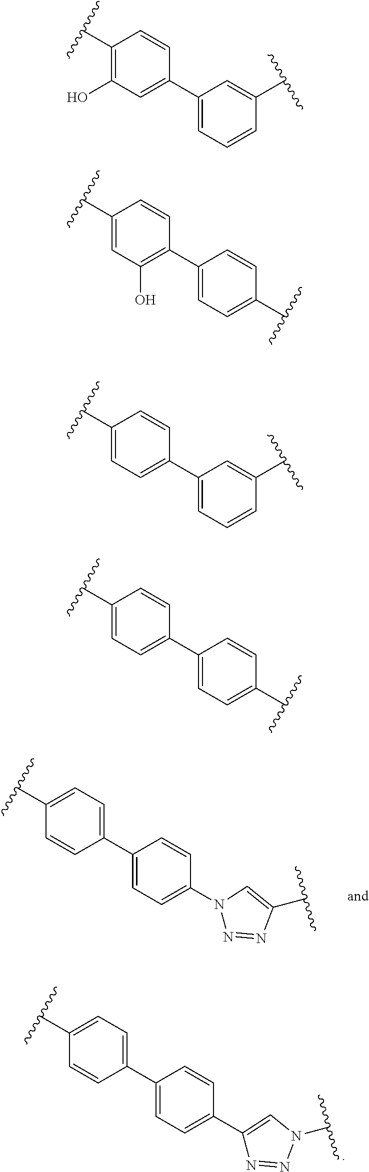 [SMILES: *c1cccc(-c2ccc(*)c(O)c2)c1 ; *c1ccc(-c2ccc(*)cc2O)cc1 ; *c1ccc(-c2cccc(*)c2)cc1 ; *c1ccc(-c2ccc(*)cc2)cc1 ; *c1ccc(-c2ccc(-n3cc(*)nn3)cc2)cc1 ; *c1ccc(-c2ccc(-c3cn(*)nn3)cc2)cc1]

In some embodiments of formula (III)-(XIII), -A-Z³— is selected from:

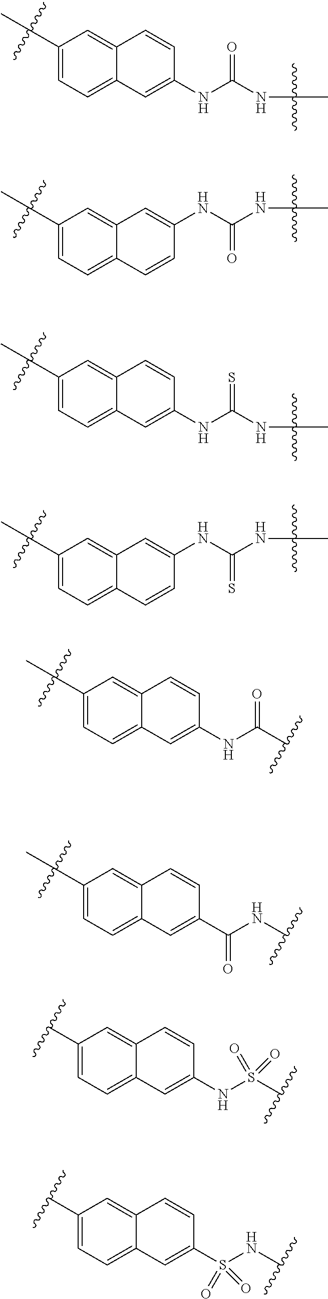

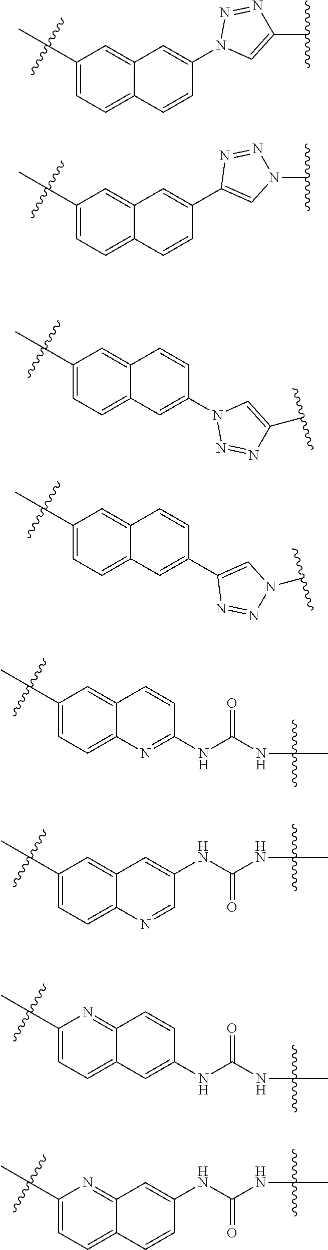

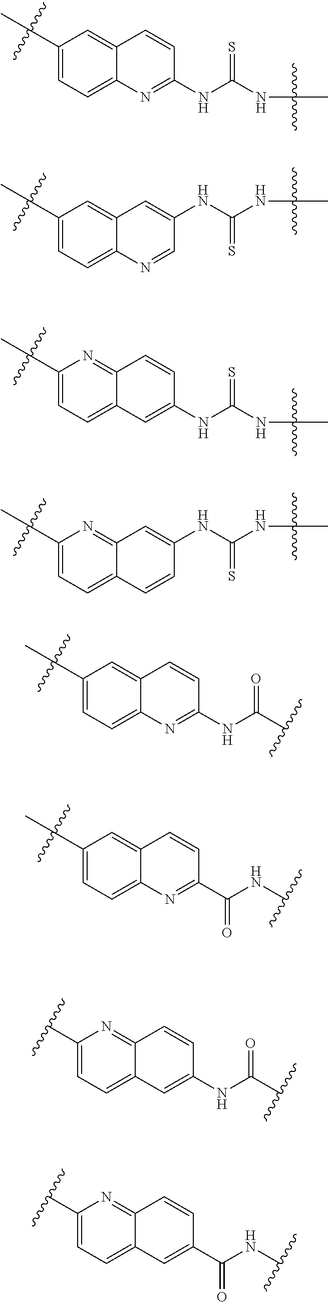

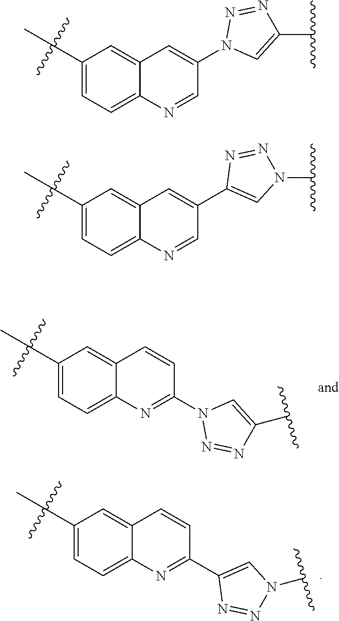 [SMILES: *c1ccc2ncc(-n3cc(*)nn3)cc2c1 ; *c1ccc2ncc(-c3cn(*)nn3)cc2c1 ; *c1ccc2nc(-n3cc(*)nn3)ccc2c1 ; *c1ccc2nc(-c3cn(*)nn3)ccc2c1]

In some embodiments of formula (III)-(XIII), Z² is O.

In some embodiments of formula (III)-(XIII), Z² is S.

In some embodiments of formula (III)-(XIII), Z² is —NR²¹—.

In some embodiments of formula (III)-(XIII), Z² is —C(R²²)₂—, wherein each R²² is independently selected from H, halogen (e.g., F) and optionally substituted (C₁-C₆)alkyl. In some embodiments, Z² is —CH₂—. In some embodiments, Z² is —CHF—. In some embodiments, Z² is —CF₂—.

In some embodiments of formula (III)-(XIII), Z²-A-Z³— is

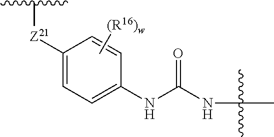

wherein:
Z¹ is O, S, or —C(R²²)₂—;
R¹⁶ is OH or CH₃; and
w is 0 to 4 (e.g., w is 0, 1, or 2).

In some embodiments, Z²¹ is S or O. In some embodiments, Z²¹ is —CH₂—. In some embodiments, Z²¹ is —CHF—. In some embodiments, Z²¹ is —CF₂—. In some embodiments, R¹⁶ is OH and w is 1. In some embodiments, R¹⁶ is CH₃ and w is 1. In some embodiments, w is 0.

In some embodiments of formula (III)-(XIII), —Z²-A-Z³— is:

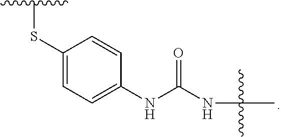 [SMILES: *NC(=O)Nc1ccc(S*)cc1]

In some embodiments of formula (III)-(XIII), —Z²-A-Z³— is

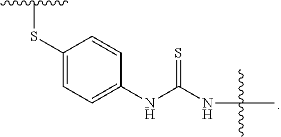 [SMILES: *NC(=S)Nc1ccc(S*)cc1]

In some embodiments of formula (III)-(XIII), —Z²-A-Z³— is

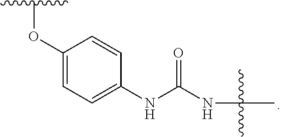 [SMILES: *NC(=O)Nc1ccc(O*)cc1]

In some embodiments of formula (III)-(XIII), —Z²-A-Z³— is

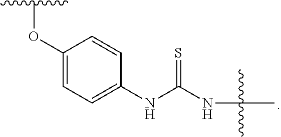 [SMILES: *NC(=S)Nc1ccc(O*)cc1]

In some embodiments of formula (III)-(XIII), —Z²-A-Z³— is

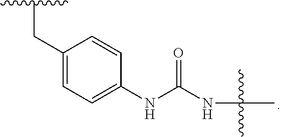 [SMILES: *Cc1ccc(NC(=O)N*)cc1]

In some embodiments of formula (III)-(XIII), —Z²-A-Z³— is

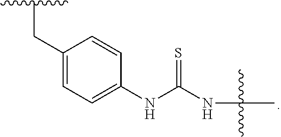 [SMILES: *Cc1ccc(NC(=S)N*)cc1]

### Prodrugs

Aspects of this disclosure include prodrugs of any of the ASGPR and M6PR binding moieties described herein that are incorporated into the compounds and conjugates of this disclosure.

The term “prodrug” refers to an agent which is converted into the drug in vivo by some physiological or chemical process (e.g., a prodrug on being brought to the physiological pH is converted to the desired drug form).

Prodrugs forms of any of the ASGPR or M6PR binding moieties described herein can be useful because, for example, can lead to particular therapeutic benefits as a consequence of an extension of the half-life of the resulting compound or conjugate in the body or a reduction in the active dose required.

Pro-drugs can also be useful in some situations, as they may be easier to administer than the parent drug. They may, for instance, be bioavailable by oral administration whereas the parent drug is not. The pro-drug may also have improved solubility in pharmacological compositions over the parent drug.

Prodrug derivative of a ASGPR or M6PR binding moiety generally includes a promoiety substituent at a suitable labile site of the compound. The promoiety refers to the group that is removed by enzymatic or chemical reactions, when a prodrug is converted to the drug in vivo.

In some embodiments, the promoiety is a group attached via an ester linkage to a hydroxyl group of the compound or drug.

In some embodiments, a prodrug derivative of one or more of the hydroxyl groups of the sugar ring may be incorporated into the compounds. For example, an ester promoiety can be incorporated at one or more of the hydroxyl groups at the 3 and/or 4 positions of the sugar (e.g., as described herein). In some embodiments, the hydroxyl groups at the 3 and 4 positions of the sugar are cyclically linked to form a promoiety (e.g., as described herein).

### Linkers

The terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), n¬butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest (Y), e.g., a biomolecule (e.g., an antibody) and the other for conjugation to a moiety (noted as X) that binds to a ASGPR cell surface receptor. When Y is a polypeptide, the polypeptide conjugation reactive terminus of the linker is in some cases a site that is capable of conjugation to the polypeptide through a cysteine thiol or lysine amine group on the polypeptide, and so is can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, heteroaryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure. In certain cases, the linker comprises one or more heteroaryl cyclic structures, e.g., a triazole, such as a 1,2,3-traizole.

In certain embodiments, L is between about 10 Å and about 20 Å in length. In certain embodiments, L is between about 15 Å and about 20 Å in length. In certain embodiments. L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to X, an optionally substituted heteroarylene linked to X, an alkylene group linked to X, or a heteroatom linked to X. In certain embodiments, L is a linker between about 10 Å and about 500 Å, which comprises an optionally substituted arylene linked to X, or optionally substituted heteroarylene linked to X, an alkylene group linked to X, or a heteroatom linked to X. In certain embodiments, L is a linker between about 10 Å and about 400 Å, which comprises an optionally substituted arylene linked to X, or optionally substituted heteroarylene linked to X, an alkylene group linked to X, or a heteroatom linked to X. In certain embodiments, L is a linker between about 10 Å and about 200 Å, which comprises an optionally substituted arylene linked to X, or optionally substituted heteroarylene linked to X, an alkylene group linked to X, or a heteroatom linked to X.

In certain embodiments, linker L separates X and Y (or Z¹) by a chain of 4 to 500 consecutive atoms. In certain embodiments, linker L separates X and Y (or Z¹) by a chain of 4 to 50 consecutive atoms. In certain embodiments, linker L separates X and Y (or Z¹) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z¹) by a chain of 6 to 50 consecutive atoms. In certain embodiments, linker L separates X and Y (or Z¹) by a chain of 11 to 50 consecutive atoms. In certain embodiments, linker L separates X and Y (or Z¹) by a chain of 16 to 50 consecutive atoms. In certain embodiments, linker L separates X and Y (or Z¹) by a chain of 21 to 50 consecutive atoms. In certain embodiments, linker L separates X and Y (or Z¹) by a chain of 26 to 50 consecutive atoms. In certain embodiments, linker L separates X and Y (or Z¹) by a chain of 31 to 50 consecutive atoms. In certain embodiments, linker L separates X and Y (or Z¹) by a chain of 36 to 50 consecutive atoms. In certain embodiments, linker L separates X and Y (or Z¹) by a chain of 41 to 50 consecutive atoms. In certain embodiments, linker L separates X and Y (or Z¹) by a chain of 46 to 50 consecutive atoms.

In certain embodiments, linker L separates X and Y (or Z¹) by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L is a chain of 5 to 500 consecutive atoms separating X and Y (or Z¹) and which comprises an optionally substituted arylene linked to X, optionally substituted heteroarylene linked to X, optionally substituted alkylene linked to X, or heteroatom linked to X. In certain embodiments, linker L is a chain of 7 to 500 consecutive atoms separating X and Y (or Z¹) and which comprises an optionally substituted arylene linked to X, optionally substituted heteroarylene linked to X, optionally substituted alkylene linked to X, or heteroatom linked to X. In certain embodiments, linker L is a chain of 10 to 500 consecutive atoms separating X and Y (or Z¹) and which comprises an optionally substituted arylene linked to X, optionally substituted heteroarylene linked to X, optionally substituted alkylene linked to X, or heteroatom linked to X. In certain embodiments, linker L is a chain of 15 to 400 consecutive atoms separating X and Y (or Z¹) and which comprises an optionally substituted arylene linked to X, optionally substituted heteroarylene linked to X, optionally substituted alkylene linked to X, or heteroatom linked to X.

In certain embodiments, linker L is a chain of 5 to 500 consecutive atoms separating X and Y (or Z¹) and which comprises an alkylene, a heteroatom, or optionally substituted heteroarylene linked to X. In certain embodiments, linker L is a chain of 7 to 500 consecutive atoms separating X and Y (or Z¹) and which comprises an alkylene, a heteroatom, or optionally substituted heteroarylene linked to X. In certain embodiments, linker L is a chain of 10 to 500 consecutive atoms separating X and Y (or Z¹) and which comprises an comprises an alkylene, a heteroatom, or optionally substituted heteroarylene linked to X. In certain embodiments, linker L is a chain of 15 to 400 consecutive atoms separating X and Y (or Z¹) and which comprises an alkylene, a heteroatom, or optionally substituted heteroarylene linked to X.

In certain embodiments, linker L is a chain of 5 to 500 consecutive atoms separating X and Y (or Z¹) and which comprises an optionally substituted triazole linked to X. In certain embodiments, linker L is a chain of 7 to 500 consecutive atoms separating X and Y (or Z¹) and which comprises an optionally substituted triazole linked to X. In certain embodiments, linker L is a chain of 10 to 500 consecutive atoms separating X and Y (or Z) and which comprises an optionally substituted triazole linked to X. In certain embodiments, linker L is a chain of 15 to 400 consecutive atoms separating X and Y (or Z) and which comprises an optionally substituted triazole linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted alkylene linked to X, or a heteroatom linked to X.

It is understood that the linker may be considered as connecting directly to a Z¹ group of a ASGPR binding moiety (X) (e.g., as described herein). In some embodiments of any of formulae (Ia)-(Ip), the linker may be considered as connecting directly to the Z¹ group. Alternatively, the —Z¹-L¹- group (e.g., as described herein) can be considered part of a linking moiety that connects L to Y. The disclosure is meant to include all such configurations of ASGPR binding moiety (X) and linker (L).

In some embodiments of formula (I)-(Ia), L is a linker of formula (VI):

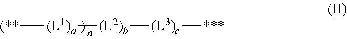

wherein
L¹ and L³ are independently a linker, and L² is a branched linking moiety, wherein L¹ to L³ together provide a linear or branched linker between X and Y;
a, b and c are independently 0 or 1;
** represents the point of attachment to L¹ of X via Z¹; and
*** represents the point of attachment to Y;
   wherein:
when n is 1, a is 1, and b is 0;
when n is >1, a is 1, and b is 1.

In some embodiments of formula (I), L is a linker of formula (XI):

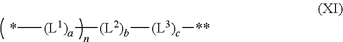

wherein
each L¹ and L³ are independently a linear linking moiety, and L² is a branched linking moiety, wherein L¹ to L³ together provide a linear or branched linker between X and Y—B;
a, b and c are independently 0 or 1;
* represents the point of attachment of L¹ to X via Z¹; and
** represents the point of conjugation of the linker L to Y—B;
wherein:
when n is 1, b is 0 and at least one of a and c is 1; and
when n is 2 or 3, a, b and c are each 1.

In some embodiments of the linker of formula (XI), n is 1, a is 1, b is 0, and c is 1, such that the linker L is of formula (XIa):

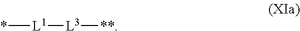

In some embodiments of the linker of formula (XI), n is 1, a is 1, b is 0, and c is 0, such that the linker L is of formula:

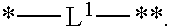

In certain embodiments, the linear linker of formula (XIa) has a backbone of 10 or more consecutive atoms covalently linking X to Y—B via Z¹, such as a backbone of 12 or more consecutive atoms, 14 or more consecutive atoms, or 16 or more consecutive atoms, and in some cases, up to 100 consecutive atoms. In certain embodiments of formula (XIa), the linear linker separates X (or Z¹) and Y—B by a chain of 20 to 50 consecutive atoms. In certain embodiments of formula (Xa), the linear linker separates X (or Z¹) and Y—B by a chain of 30 to 60 consecutive atoms.

In some embodiments of the linker of formula (XI), n is 2, a is 1, b is 1, and c is 1, such that the linker L is of formula (XIb):

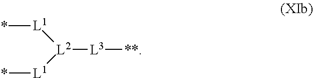

In some embodiments of the linker of formula (XI), n is 3, a is 1, b is 1, and c is 1, such that the linker L is of formula (XIc):

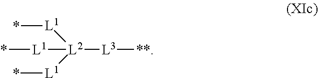

In some embodiments of the linker of any one of formulae (XI) or (XIa)-(XIc), each L¹ is of formula (XII):

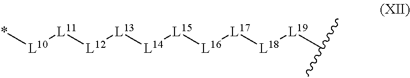

wherein:
L¹⁰ is a linking moiety, and * represents the point of attachment of L¹ to X via Z¹; and
L¹¹ to L¹⁹ are independently absent or a linking moiety,
wherein L¹⁰ to L¹⁹ of each L¹ are each independently selected from —C₁₋₆-alkylene-, —C₁₋₁₂-alkylene-, —C₁₋₂₀-alkylene-, —NHCO—C₁₋₆-alkylene-, —CONH—C₁₋₆-alkylene-, —NH—C₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ, —(OCH₂CH₂)ₚ—, —NHCO—, —CONH—, —NHSO₂—, —SO₂NH—, —NHCONH—, —NHCSNH—, —CO—, —SO₂—, —O—, —S—, pyrrolidine-2,5-dione, 1,2,3-triazole, —NH—, —N(C₁₋₆-alkyl)-, and —N(CH₃)—, wherein each p is independently 1 to 50, such as 1 to 20, 1 to 12, 1 to 10, 1 to 8, or 1 to 6, e.g., 1, 2, 3, 4, 5 or 6.

In certain embodiments of formula (XII), the linking moiety L¹ includes a linear backbone of 6 to 40 consecutive atoms, such as 10 to 40, 10 to 30, 16 to 30, or 20 to 30 consecutive atoms. In certain embodiments of formula (XII), the linking moiety L¹ includes a linear backbone of each L¹ comprises a linear backbone of 6 to 20 consecutive atoms, such as 6 to 16 consecutive atoms, such as 8, 9, 10, 11, 12, 13, 14, 15 or 16 consecutive atoms.

In certain embodiments, the linking moiety of formula (XII) includes one or repeating ethylene glycol moieties (e.g., —CH₂CH₂O— or —OCH₂CH₂—). In certain cases, the linking moiety of formula (XII) includes 1 to 10 ethylene glycol moieties, such as 1, 2, 3, 4, 5 or 6 ethylene glycol moieties.

In certain embodiments, the linking moiety of formula (XII) includes one or more triazole (e.g., 1,2,3-triazole) containing linking moieties. It is understood that the triazole may be derived from an azido-alkyne click chemistry and thus have two possible orientations depending on the method of synthesis:

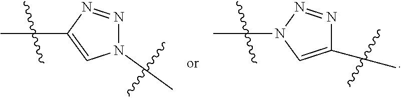 [SMILES: *c1cn(*)nn1 ; *c1cn(*)nn1]

In certain embodiments, the triazole containing linking moiety is:

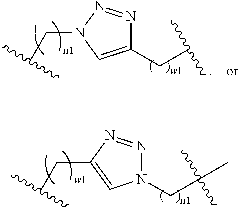

wherein w1 and u1 are independently 0 to 12, such as 0, 1, 2, 3, 4, 5 or 6.

In some embodiments of the linker of formula (XI), b is 1 and L² is of the formula (XIIIa) or (XIIIb):

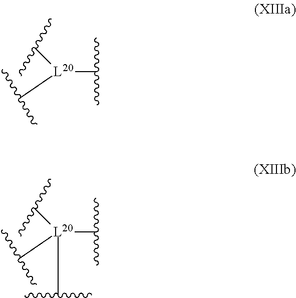

wherein:
L²⁰ is a branched linking moiety including one or more linking moieties independently selected from amino acid residue (e.g., a residue such as Gly, Ala, beta-Ala, Lys, Om, Asp, Glu, Ser, Cys, or a derivative thereof), —NH—CH[(CH₂)_{q}]₂O— or —NH—C[(CH₂)_{q}]₃O—,

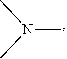

-C₁₋₆-alkylene-, —NHCO—, —CONH—, —NHSO₂—, —SO₂NH—, —CO—, —SO₂—, —O—, —S—, pyrrolidine-2,5-dione, 1,2,3-triazole, —NH—, and -Nme-, —NHC(═O)NH—, —NHC(═S)NH—, —O(CH₂)ₚ—, and —(OCH₂CH₂)ₚ—;
wherein each p is independently 1 to 50, and q is 1-6.

In some embodiments of the linker of formula (XI), b is 1 and the linking moiety L² is selected from one of (L2A)-(L2D):

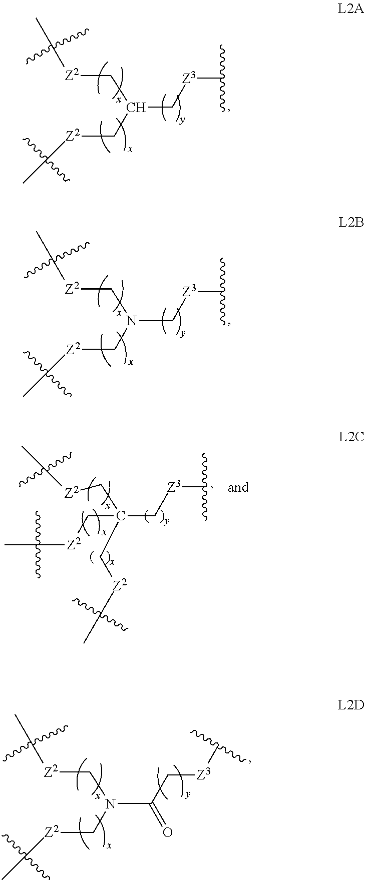

wherein:
each Z² and Z³ is independently absent or selected from —NHCO—, —CONH—, —CO—, —O—, —NH—, and -Nme-;
x is 1 to 12 (e.g., 1 to 6, or 1 to 3); and
y is 0 to 12 (e.g., 1 to 6, or 1 to 3).

In some embodiments of any one of L2A-L2D, Z² is —NHCO—. In some embodiments of any one of L2A-L2D, Z² is —CONH—. In some embodiments of any one of L2A-L2D, Z² is —CO—. In some embodiments of any one of L2A-L2D, Z² is —O—. In some embodiments of any one of L2A-L2D, Z^{Z} is NH—. In some embodiments of any one of L2A-L2D, Z² is -Nme-. In some embodiments of any one of L2A-L2D, Z² is absent.

In some embodiments of any one of L2A-L2D. Z³ is —NHCO—. In some embodiments of any one of L2A-L2D, Z³ is —CONH—. In some embodiments of any one of L2A-L2D, Z³ is —CO—. In some embodiments of any one of L2A-L2D. Z³ is —O—. In some embodiments of any one of L2A-L2D, Z³ is —NH—. In some embodiments of any one of L2A-L2D, Z³ is -Nme-. In some embodiments of any one of L2A-L2D, Z³ is absent.

In some embodiments of L2A, Z² is —O—, y is 0 and the linking moiety is of the structure L2Ai:

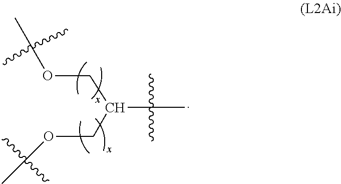

In some embodiments of L2B, Z² is —O— or —CO—, and the linking moiety is of the structure L2Bi or L2Bii:

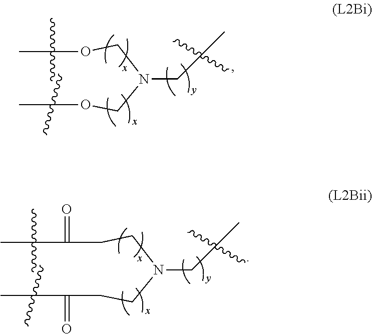

In some embodiments of L2C, Z² is —O—, —CO—, —NHCO—, or —NH—, and the linking moiety is of the structure L2Ci, L2Cii, L2Ciii, or L2Civ:

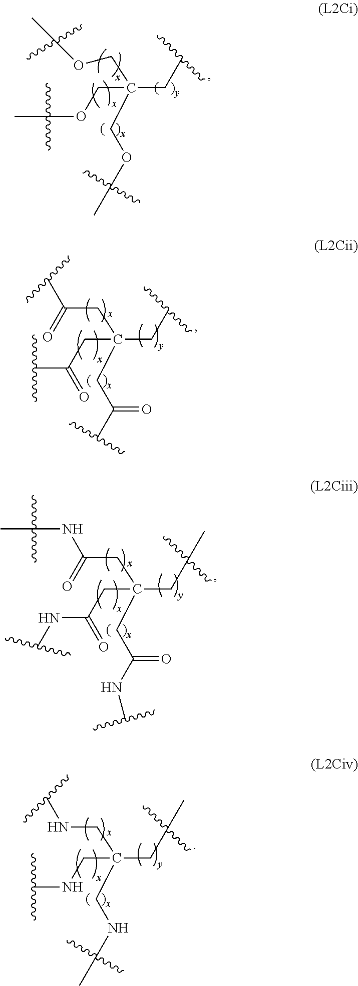

In some embodiments of L2D, Z² is absent and the linking moiety is of the structure L2Di:

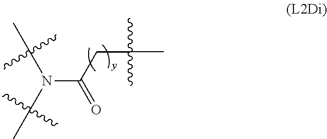

In some embodiments, of any one of formulae L2A-L2Di, x is 1 to 6. In some cases, x is 1 to 3. In some cases, x is 1. In some cases, x is 2. In some cases, x is 3.

In some embodiments of any one of formulae L2A-L2Di, y is 0 to 6. In some cases, y is 0 to 3. In some cases, y is 0. In some cases, y is 1. In some cases, y is 2. In some cases, y is 3.

In certain embodiments of formula (XI), b is 1 and the linking moiety L² is selected from:

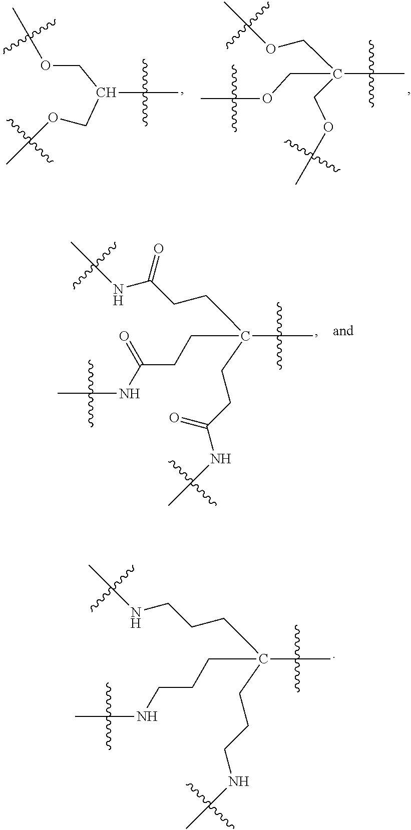

In some embodiments of the linker of formula (XI), b is 1 and the linking moiety L² is of the formula (XIV):

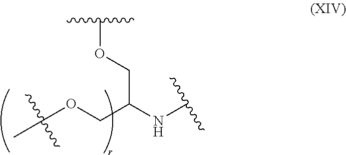

wherein:
r is 1 or 2; and
when n is 2, r is 1,
when n is 3, r is 2.

In some embodiments of the linker of formula (XI), b is 1 and the linking moiety L² is of the formula (Xva) or (XVb):

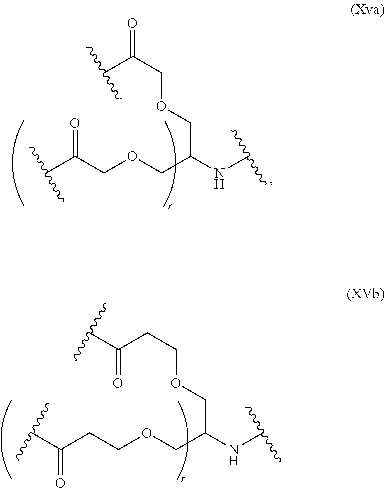

wherein:
r is 1 or 2; and
when n is 2, r is 1,
when n is 3, r is 2.

In some embodiments L² is of formula (XIIa) or (XIIIb) and L² includes two 2 or more amino acid residues (e.g., 3 or more, or 4 or more amino acid residues, linear or dendrimer). In some embodiments. L² includes 4 or more amino acid residues that are branched linking moieties selected from Lys, Orn, Asp, Glu, Ser, and Cys (e.g., where the sidechain, amino and carboxylic acid are each linked to an adjacent moiety).

In some embodiments of the linker of any one of formulae (XI) or (Xa)-(Xc), each L³ is of the formulae (XVI):

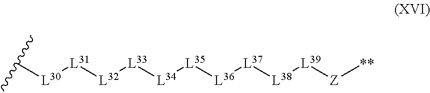

wherein:
L³⁰ to L³⁹ are independently absent or a linking moiety; and
Z is a residual moiety resulting from the covalent linkage of a chemoselective ligation group of the linker to a compatible group of Y—B;
wherein L³⁰ to L³⁹ are each independently selected from —C₁₋₂₀-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —NHCONH—, —NHCSNH—, —CO—, —SO₂—, —O—, —S—, pyrrolidine-2,5-dione, 1,2,3-triazole, —NH—, and -Nme-, wherein each p is independently 1 to 50.

In certain embodiments, the linking moiety of formula (XVI) includes a linear backbone of 6 to 40 consecutive atoms, such as 10 to 40, 10 to 30, or 20 to 30 consecutive atoms.

In certain embodiments, the linking moiety of formula (XVI) includes repeating ethylene glycol moieties (e.g., —CH₂CH₂O— or —OCH₂CH₂—). In certain cases, the linking moiety of formula (XVI) includes 2 to 20 ethylene glycol moieties, such as 2 to 15, 2 to 10, 3 to 20, 3 to 15, 3 to 10, 4 to 15, 5 to 15 or 5 to 10 ethylene glycol moieties. In some instances, the linking moiety of formula (XVI) includes 2 or more ethylene glycol moieties, such as 3 or more, 4 or more, 5 or more, 6 or more, 7 or more, or even more ethylene glycol moieties.

In certain embodiments, the linking moiety of formula (XVI) includes one or more triazole linking moieties. In some instances, the linker includes one or more 1,2,3-triazole linking moieties. In certain cases, the one or more 1,2,3-triazoel moieties is selected from one of the following structures:

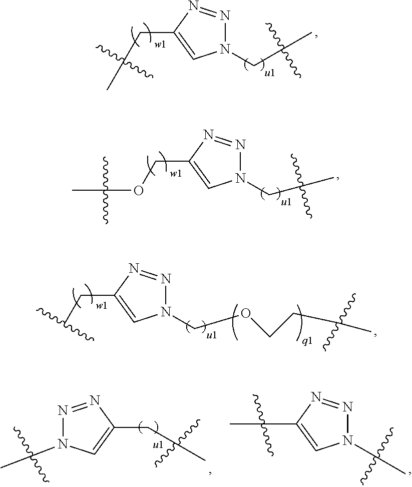

wherein w1, u1 and q1 are independently 1 to 25 (e.g., 1 to 12, such as 1 to 6).

In certain embodiments, the linking moiety L³ includes (C₁₀-C₂₀-alkylene (e.g., C₁₂-alkylene), or —(OCH₂CH₂)ₚ—, where p is 1 to 25, such as 3 to 25, 5 to 24, 7 to 25, 10 to 25, 15 to 25 or 20 to 24.

In some embodiments, the linker L is of formula XVII:

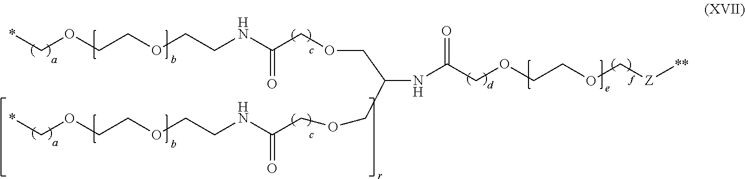

wherein:
a is 0 to 12 (e.g., 2 to 6, or 2, or 3);
b is 1 to 6 (e.g., 1, 2, or 3);
c is 1 to 6 (e.g., 1, 2, or 3);
r is 1 or 2;
d is 1 to 6 (e.g., 1, 2, or 3);
e is b is 1 to 6 (e.g., 1, 2, or 3);
f is 1 to 6 (e.g., 1, 2, or 3); and
Z is a residual moiety resulting from the covalent linkage of a chemoselective ligation group (e.g., as described herein) of a linker precursor to a compatible group of Y—B.

In some embodiments of the formula XVII, Z is a residual moiety resulting from the covalent linkage (e.g., via a thioether bond) of a thiol-reactive chemoselective ligation group to one or more cysteine residue(s) of Y—B. In some embodiments, the thiol-reactive chemoselective ligation group includes maleimide, bromomaleimide, haloacetamide, vinyl sulfone, or thiolactone. In some embodiments, the thiol-reactive group is selected from one of the following structures:

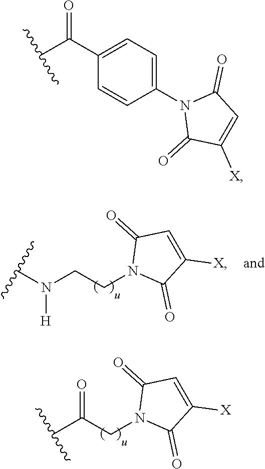

wherein:
u is 1 to 11 (e.g., 1 to 5);
v is 1 to 11 (e.g., 1 to 5); and
X is H or Br.

In some embodiments of formula XVII, Z is a residual moiety resulting from the covalent linkage (e.g., via an amide bond) of an amine-reactive chemoselective ligation group to one or more lysine residue(s) of Y—B. In some embodiments, the amine-reactive chemoselective ligation group includes an active ester (e.g., N-hydroxysuccinimidyl (NHS) ester, sulfo-NHS ester, pentafluorophenyl (PFP) ester, tetrafluorophenyl (TFP) ester, or the like).

In some embodiments, the linker L includes one of (XVIIIa)-(XVIIIc):

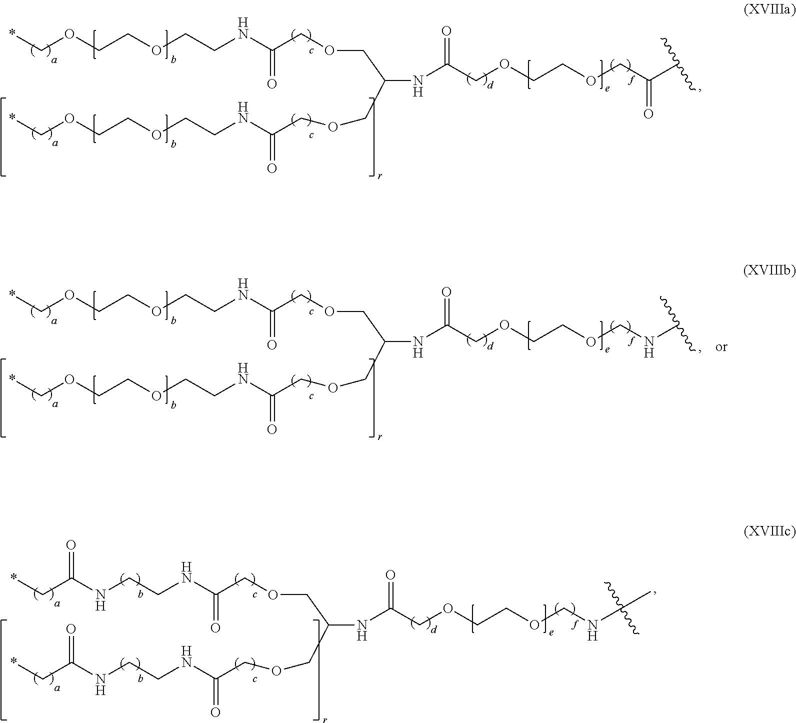

wherein:
a is 0 to 12 (e.g., 2 to 6, or 2, or 3);
b is 1 to 6 (e.g., 1, 2, or 3);
c is 1 to 6 (e.g., 1, 2, or 3);
r is 1 or 2:
d is 1 to 6 (e.g., 1, 2, or 3);
e is b is 1 to 6 (e.g., 1, 2, or 3); and
f is 1 to 6 (e.g., 1, 2, or 3).

In some embodiments of any one of formulae (XVII) or (XVIIIa)-(XVIIIc), a is 2 to 6, such as 2 to 3. In some embodiments, a is 2. In some embodiments, a is 3. In some embodiments, a is 4. In some embodiments, a is 5. In some embodiments a is 6.

In some embodiments of any one of formulae (XVII) or (XVIIIa)-(XVIIIc), b is 1 to 4, such as 1 to 3. In some embodiments, b is 1. In some embodiments, b is 2. In some embodiments, b is 3.

In some embodiments of any one of formulae (XVII) or (XVIIIa)-(XVIIIc), c is 1 to 4, such as 1 to 3. In some embodiments, c is 1. In some embodiments, c is 2. In some embodiments, c is 3.

In some embodiments of any one of formulae (XVII) or (XVIIIa)-(XVIIIc), r is 1. In some embodiments, r is 2.

In some embodiments of any one of formulae (XVII) or (XVIIIa)-(XVIIIc), d is 1 to 4, such as 1 to 3. In some embodiments, d is 1. In some embodiments, d is 2. In some embodiments, d is 3.

In some embodiments of any one of formulae (XVII) or (XVIIIa)-(XVIIIc), e is 1 to 5, such as 1 to 3. In some embodiments, e is 1. In some embodiments, e is 2. In some embodiments, e is 3. In some embodiments, e is 4. In some embodiments, e is 5.

In some embodiments of any one of formulae (XVII) or (XVIIIa)-(XVIIIc), f is 1 to 4, such as 1 to 3. In some embodiments, f is 1. In some embodiments, f is 2. In some embodiments, f is 3.

In some embodiments of any one of formulae (XVII) or (XVIIIa)-(XVIIIc), a is 1-4; b is 1-4; c is 1-3; r is 1; d is 1-3; e is 1-6; and f is 1-3.

In some embodiments of any one of formulae (XVII) or (XVIIIa)-(XVIIIc), a is 1-4; b is 1-4; c is 1-3; r is 2; d is 1-3; e is 1-6; and f is 1-3.

In some embodiments of any one of formulae (XVII) or (XVIIIa)-(XVIIIc), a is 2; b is 1; c is 2; r is 1; d is 2; e is 3; and f is 2.

In some embodiments of any one of formulae (XVII) or (XVIIIa)-(XVIIIc), a is 2; b is 1; c is 2; r is 2; d is 2; e is 3; and f is 2.

In some embodiments of any one of formulae (XVII) or (XVIIIa)-(XVIIIc), a is 4; b is 1; c is 2; r is 1; d is 2; e is 3; and f is 2.

In some embodiments of any one of formulae (XVII) or (XVIIIa)-(XVIIIc), a is 4; b is 1; c is 2; r is 2; d is 2; e is 3; and f is 2.

In some embodiments of any one of formulae (XVII) or (XVIIIa)-(XVIIIc), a is 2; b is 2; c is 2; r is 1; d is 2; e is 3; and f is 2.

In some embodiments of any one of formulae (XVII) or (XVIIIa)-(XVIIIc), a is 2; b is 2; c is 2; r is 2; d is 2; e is 3; and f is 2.

In some embodiments of any one of formulae (XVII) or (XVIIIa)-(XVIIIc), a is 0; b is 3; c is 2; r is 2; d is 2; e is 3; and f is 2.

In some embodiments of any one of formulae (XVII) or (XVIIIa)-(XVIIIc), a is 2; b is 4; c is 2; r is 2; d is 2:e is 3; and f is 2.

In some embodiments of any one of formulae (XVII) or (XVIIIa)-(XVIIIc), a is 2; b is 4; c is 2; r is 1; d is 2; e is 3; and f is 2.

In some embodiments, the linker L includes L^{A}:

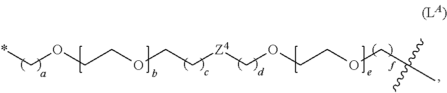

wherein:
Z⁴ is selected from —NHC(O)NH—, —NHC(O)—, —C(O)NH—, —O—, —NH—;
a is 0 to 12 (e.g., 2 to 6, or 2, or 3);
b is 1 to 6 (e.g., 1, 2, or 3);
c is 1 to 6 (e.g., 1, 2, or 3);
d is 1 to 6 (e.g., 1, 2, or 3);
e is b is 1 to 6 (e.g., 1, 2, or 3); and
f is 1 to 6 (e.g., 1, 2, or 3).

In some embodiments of L^{A}, Z⁴ is —NHC(O)NH—. In some cases, Z⁴ is —NHC(O)—. In some cases, Z⁴ is —C(O)NH—. In some cases, Z⁴ is —O—. In some cases, Z⁴ is —NH—.

In some embodiments of L^{A}, a is 1-4; b is 1-4; c is 1-3; d is 1-3; e is 1-6; and f is 1-3. In some embodiments, a is 4; b is 1; c is 2; d is 2; e is 5; and f is 2.

In some embodiments, Z⁴ is —NHC(O)NH— and a is 1-4; b is 1-4; c is 1-3; r is 1; d is 1-3; e is 1-6; and f is 1-3. In some embodiments, Z⁴ is —NHC(O)— and a is 1-4; b is 1-4; c is 1-3; r is 1; d is 1-3; e is 1-6; and f is 1-3.

In some embodiments, the linker L includes L^{B}:

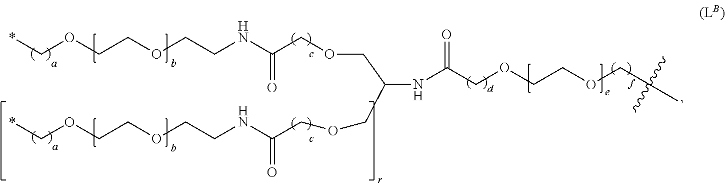

wherein:
a is 0 to 12 (e.g., 2 to 6, or 2, or 3);
b is 1 to 6 (e.g., 1, 2, or 3);
c is 1 to 6 (e.g., 1, 2, or 3);
r is 1 or 2;
d is 1 to 6 (e.g., 1, 2, or 3);
e is b is 1 to 6 (e.g., 1, 2, or 3); and
f is 1 to 6 (e.g., 1, 2, or 3).

In some embodiments of L^{B}, a is 1-4; b is 1-4; c is 1-3; r is 1; d is 1-3; e is 1-6; and f is 1-3. In some embodiments, a is 4; b is 1; c is 2; r is 1; d is 2; e is 5; and f is 2. In some embodiments, a is 2; b is 1; c is 2; r is 1; d is 2; e is 3; and f is 2. In some embodiments, a is 4; b is 1; c is 2; r is 1; d is 2; e is 3; and f is 2. In some embodiments, a is 1; b is 2; c is 2; r is 1; d is 2; e is 3; and f is 2. In some embodiments, a is 0; b is 3; c is 2; r is 1; d is 2; e is 3; and f is 2.

In some embodiments of L^{B}, a is 1-4; b is 1-4; c is 1-3; r is 2; d is 1-3; e is 1-6; and f is 1-3. In some embodiments, a is 2; b is 1; c is 2; r is 2; d is 2; e is 3; and f is 2. In some embodiments, a is 4; b is 1; c is 2; r is 2; d is 2; e is 3; and f is 2. In some embodiments, a is 1; b is 2; c is 2; r is 2; d is 2; e is 3; and f is 2. In some embodiments, a is 0; b is 3; c is 2; r is 2; d is 2; e is 3; and f is 2.

In some embodiments, the linker L includes L^{C}:

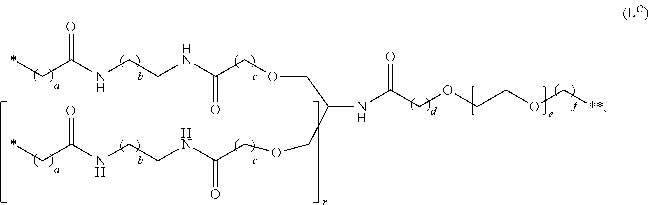

wherein:
a is 0 to 12 (e.g., 1 to 6, 2 to 6, or 2, or 3);
b is 1 to 6 (e.g., 1 to 4, such as 1, 2, or 3);
c is 1 to 6 (e.g., 1 to 3, such as 1, 2, or 3);
r is 1 or 2;
d is 1 to 6 (e.g., 1 to 3, such as 1, 2, or 3);
e is b is 1 to 6 (e.g., 1, 2, or 3); and
f is 1 to 6 (e.g., 1 to 3, such as 1, 2, or 3).

In some embodiments of L^{c}, a is 1-4; b is 1-4; c is 1-3; r is 1; d is 1-3; e is 1-6; and f is 1-3. In some embodiments, a is 2; b is 4; c is 2; r is 1; d is 2; e is 5; and f is 2.

In some embodiments of L, a is 1-4; b is 1-4; c is 1-3; r is 2; d is 1-3; e is 1-6; and f is 1-3. In some embodiments, a is 2; b is 4; c is 2; r is 2; d is 2; e is 5; and f is 2.

In certain embodiments of the ASGPR binding moiety (X) as described herein, —Z¹— is linked to an -L¹- moiety (e.g., of the linker as described herein). In some embodiments, the subject compounds comprise a —Z¹-L¹- moiety comprising a linking moiety selected from:

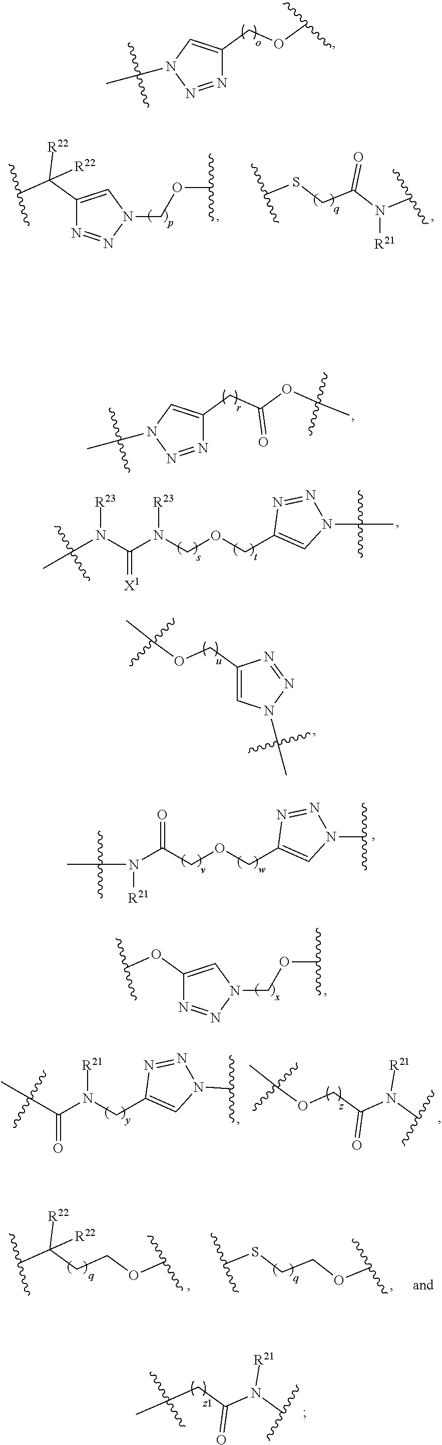

wherein each R²¹ is independently selected from H, and optionally substituted (C₁-C₆)alkyl; each R²² is independently selected from H, halogen (e.g., F) and optionally substituted (C₁-C₆)alkyl; and o, p, q, r, s, t, u, y, w, x, y, z and z1 are each independently 1 to 6.

In certain embodiments, the Z¹-L¹- group is

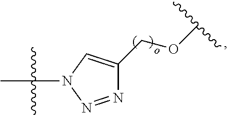

and o is 1 or 2.

In certain embodiments, the Z¹-L¹- group is

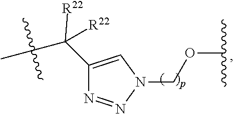

each R²² is H, and p is 1 or 2.

In certain embodiments, the Z¹-L¹- group is

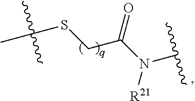

where q is 1-3.

In certain embodiments, the Z¹-L¹- group is

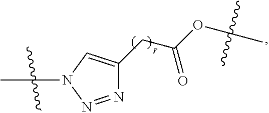

where r is 1-3.

In certain embodiments, the Z¹-L¹- group is

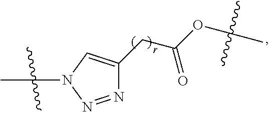

where r is 1-3.

In certain embodiments, the Z¹-L¹- group is

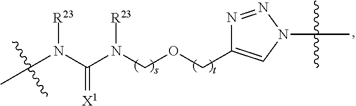

where s and t are each independently 1-3.

In certain embodiments, the Z¹-L¹- group is

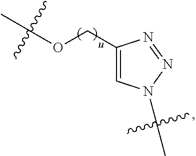

where u is 1-3.

In certain embodiments, the Z¹-L¹- group is

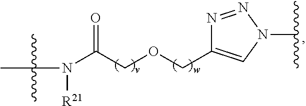

where v and w are each independently is 1-3.

In certain embodiments, the Z¹-L¹- group is

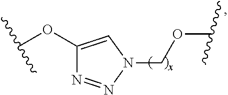

where x is 0-3.

In certain embodiments, the Z¹-L¹- group is

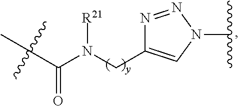

where y is 1-3.

In certain embodiments, the Z¹-L¹- group is

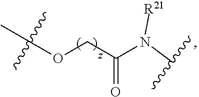

where R²¹ is H, and z is 1-4.

In certain embodiments, the Z¹-L¹- group is

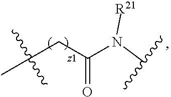

where R²¹ is H, and z1 is 1-4.

In certain embodiments, the Z¹-L¹- group is

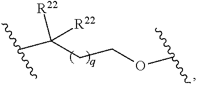

where each R²² is H, and q is 1-3.

In certain embodiments, the Z¹-L¹- group is

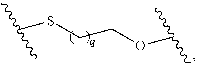

where q is 1-3.

In certain embodiments, the subject compounds comprise a —Z¹-L- group comprising a linking moiety selected from:

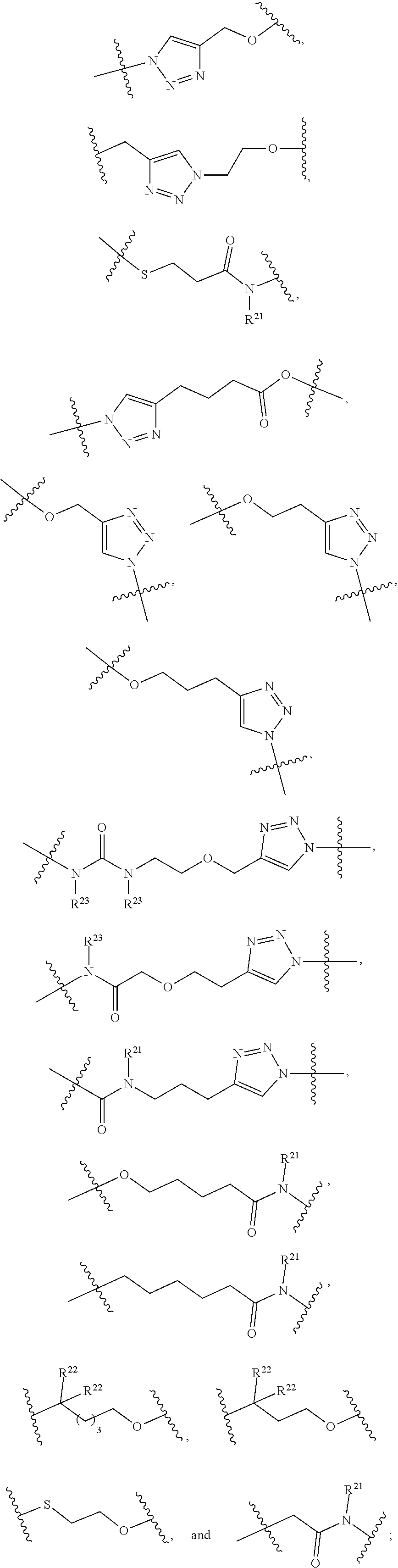

where R²¹ is independently selected from H, and optionally substituted (C₁-C₆)alkyl (e.g., methyl); and each R²² is independently selected from H, halogen (e.g., F) and optionally substituted (C₁-C₆)alkyl (e.g., methyl). In certain embodiments. R²¹ is H. In certain embodiments, each R²² is H.

In certain embodiments, the —Z¹-L¹- group is

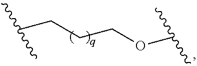

where q is 1-3. In certain cases, q is 1. In certain cases, q is 2. In certain cases, q is 3.

In certain embodiments, the —Z¹-L¹- group is

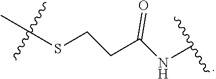 [SMILES: *NC(=O)CCS*]

In certain embodiments, —Z¹-L¹- includes an optionally substituted —NH-heteroarylene-. In certain embodiments, the heteroarylene is a triazole. In certain cases, the heteroarylene is pyridine. In certain cases, the heteroarylene is pyrimidine. In certain cases, the heteroarylene is thiadiazole.

In certain embodiments, the —Z¹-L¹- includes a group selected from:

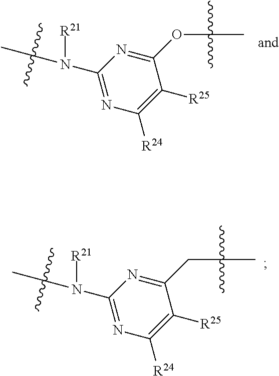

wherein R²⁴ and R²⁵ are each independently selected from H, optionally substituted C₍₁₋₆₎-alkyl, optionally substituted fluoroalkyl, and halogen; and each R²¹ is independently selected from H, optionally substituted (C₁-C₆)alkyl, and optionally substituted alkanoyl. In certain cases, R²¹ is H. In certain cases, R²⁴ is C₍₁₋₃₎-alkyl, or C₍₁₋₃₎-fluoroalkyl. In some cases, the fluoroalkyl is CF₃. In certain cases, R²¹ is C₍₁₋₃₎-alkyl, or C₍₁₋₃₎-fluoroalkyl. In some cases, the fluoroalkyl is CF₃.

In some embodiments, the linker includes a polypeptide scaffold where some or all of the sidechain groups of the amino acid residues of such a polypeptide scaffold have been modified to attach a X binding moiety (e.g., as described herein). It is understood that X binding moieties (e.g., as described herein) can be conjugated to amino acid residues, such as Asp, Lys, Orn, Glu, and Ser, of a polypeptide containing linker via a convenient conjugation chemistry. In some embodiments, the linker contains a polylysine polypeptide. In some embodiments, the linker contains a polyornithine polypeptide. In some embodiments, the linker contains a polyserine polypeptide. In some embodiments, the linker contains a polyaspartate polypeptide. The polypeptide backbone of such a linker can be a randomly polymerized polymer having an average length, or a polymer of defined length prepared e.g., in a controlled stepwise fashion. In some cases, the polypeptide linker has a length of 10-100 amino acid residues, such as 20-90, or 20-50 amino acid residues. In some embodiments, the N-terminal or C-terminal of the polypeptide linker is modified to include a linking moiety to an additional X binding moiety (e.g., as described herein). In some embodiments, the N-terminal or C-terminal of the polypeptide linker segment is modified with one or more linking moieties (e.g., as described herein) suitable for attachment to a protein construct (Y—B) including a polypeptide that specifically binds target autoantibody.

In certain embodiments of the linker of formula (II), L¹ to L³ each independently comprise one or more linking moieties independently selected from —C₁₋₂₀-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2,5-dione, piperazine or piperidine ring as described herein), amino acid residue (naturally or non-naturally occurring amino acid residue), —NH—, and -Nine-, wherein each p is independently 1 to 50.

In certain embodiments of the linker of formula (II), any of L¹-L³ comprises repeating ethylene glycol moieties (e.g., —CH₂CH₂O— or —OCH₂CH₂—). In certain cases, the linker of formula (II) comprises 1 to 25 ethylene glycol moieties, such as 3 to 25, 5 to 25, 7 to 25, 10 to 25, 15 to 25, 17 to 25, 20 to 25 or 22 to 25 ethylene glycol moieties. In some instances, the linker of formulae (II) comprises 3 or more ethylene glycol moieties, such as 5 or more, 7 or more, 10 or more, 15 or more, 20 or more, or even more ethylene glycol moieties.

In certain embodiments of the linker of formula (II), any of L¹-L³ comprises one or more triazole linking moieties. In some instances, the linker comprises one or more 1,2,3-triazole linking moieties. In certain cases, the one or more 1,2,3-triazole moieties is selected from one of the following structures:

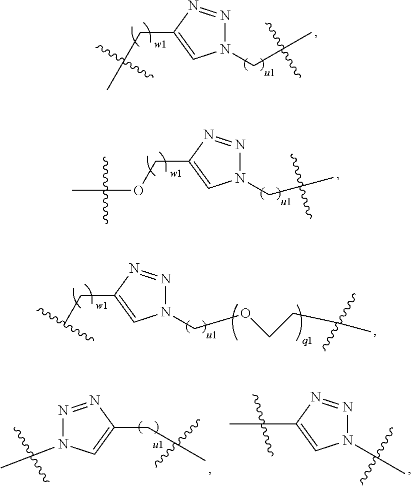

wherein w1, u1 and q1 are independently 1 to 25 (e.g., 1 to 12, such as 1 to 6).

In certain embodiments of the linker of formula (II), n is 1, such that b is 0, and the linker is of the formula (IIa):

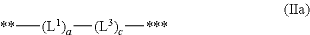

wherein
L¹ and L³ are independently a linker (e.g., as described herein), wherein L¹ to L³ together provide a linear linker between X and Y;
a is 1;
c is 0 or 1:
** represents the point of attachment to L¹ of X via Z¹; and
*** represents the point of attachment to Y.

In certain embodiments of the linker of formula (IIa), the linear linker has a backbone of 20 or more consecutive atoms covalently linking X to Y via Z, such as a backbone of 25 or more consecutive atoms, or 30 or more consecutive atoms, and in some cases, up to 100 consecutive atoms. In certain embodiments of formula (IIa), the linear linker separates X and Y (or Z¹) by a chain of 20 to 50 consecutive atoms. In certain embodiments, the linear linker separates X and Y (or Z¹) by a chain of 21 to 50 consecutive atoms, by a chain of 22 to 50 consecutive atoms, by a chain of 23 to 50 consecutive atoms, by a chain of 24 to 50 consecutive atoms, by a chain of 25 to 50 consecutive atoms, by a chain of 26 to 50 consecutive atoms, by a chain of 27 to 50 consecutive atoms, by a chain of 28 to 50 consecutive atoms, or by a chain of 29 to 50 consecutive atoms. In certain embodiments of formula (IIa), the linear linker separates X and Y (or Z¹) by a chain of 30 to 60 consecutive atoms. In certain embodiments, the linear linker separates X and Y (or Z¹) by a chain of 31 to 60 consecutive atoms. In certain embodiments, the linear linker separates X and Y (or Z¹) by a chain of 32 to 60 consecutive atoms. In certain embodiments, the linear linker separates X and Y (or Z¹) by a chain of 33 to 60 consecutive atoms. In certain embodiments, the linear linker separates X and Y (or Z¹) by a chain of 34 to 60 consecutive atoms. In certain embodiments, the linear linker L separates X and Y (or Z¹) by a chain of 35 to 50 consecutive atoms. In certain embodiments, the linear linker L separates X and Y (or Z¹) by a chain of 36 to 50 consecutive atoms. In certain embodiments, the linear linker L separates X and Y (or Z¹) by a chain of 41 to 50 consecutive atoms. In certain embodiments, the linear linker L separates X and Y (or Z¹) by a chain of 46 to 50 consecutive atoms.

In certain other embodiments of formula (II), n is 2 or more, such that L¹ to L³ together provide a branched linker between X and Y.

In certain embodiments of formula (II), n is 2 or more, and L² is selected from:

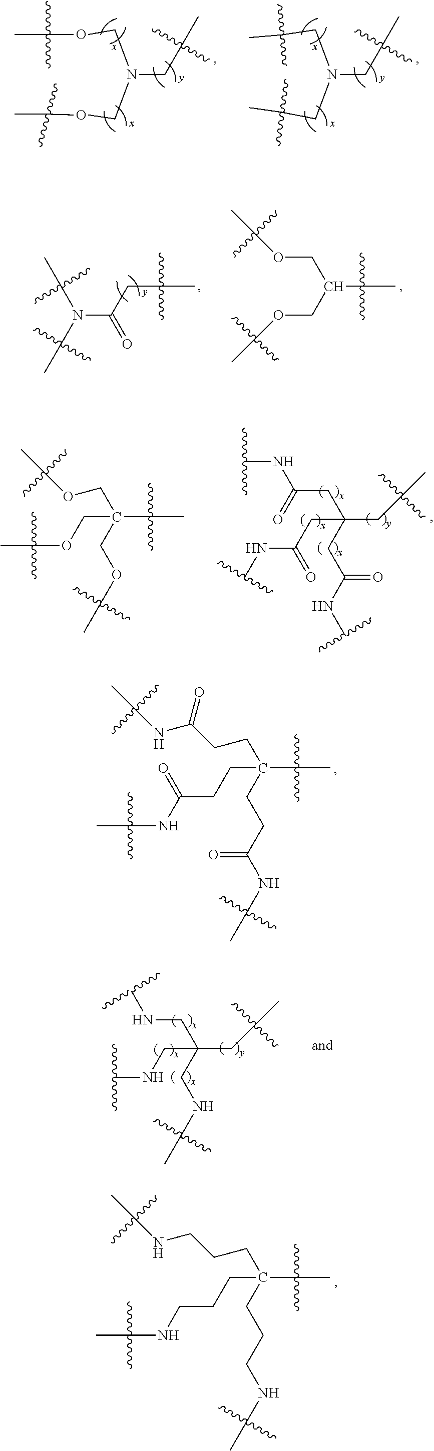

wherein each x and y are independently 1 to 10.

In certain embodiments of formula (II), L¹-L² comprises a backbone of 14 or more consecutive atoms between X and the branching atom, such as 14 to 50, 14 to 40, 14 to 35 or 14 to 30 consecutive atoms between X and the branching atom.

In certain embodiments of formula (II) or (IIa), L³ comprises a backbone of 10 to 80 consecutive atoms, such as 12 to 70, 12 to 60, or 12 to 50 consecutive atoms.

In certain embodiments of formula (II) or (IIa), wherein L³ comprises a linking moiety selected from (C₁₀-C₂₆-alkylene (e.g., C₁₂-alkylene), or —(OCH₂CH₂)ₚ—, where p is 1 to 25, such as 3 to 25, 5 to 24, 7 to 25, 10 to 25, 15 to 25 or 20 to 24.

In certain embodiments, L is of formula (IIb):

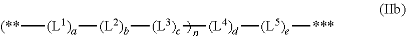

wherein
each L¹ to L⁵ is independently a linking moiety which together provide a linear or branched linker between Z and Y;
a, b, c, d, and e are each independently 0, 1, or 2;
** represents the point of attachment to L¹ of X via Z¹; and
*** represents the point of attachment to Y;
wherein:
when n is 1, a is 1, and c is 0; and
when n is >1, a is 1, and c is 1.

In certain embodiments of the linker of formula (IIb), L¹ to L⁵ each independently comprise one or more linking moieties independently selected from —C₁₋₂₀-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—(OCH₂CH₂)ₚ—, —NHCO—, —CONH—, —NHSO₂—, —SO₂NH—, —CO—, —SO₂—, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2.5-dione, piperazine or piperidine ring as described herein), amino acid residue (naturally or non-naturally occurring amino acid residue), —NH—, and -Nme-, wherein each p is independently 1 to 50.

In certain embodiments of formula (IIb), -(L¹)ₐ- comprises an optionally substituted alkyl or ethylene glycol linking moiety. In certain cases, L¹ comprises an optionally substituted —C₁₋₆-alkylene-. In certain cases, L¹ comprises an ethylene glycol linking moiety.

In certain embodiments of formula (IIb), L¹ is independently selected from: —C₁₋₆-alkylene-, —(CH₂CH₂O)ₜ—, —C₁₋₆-alkylene-NR⁴CO—, —C₁₋₆-alkyleneCONH—, or OCH₂, wherein t is 1 to 20; and R⁴ is independently selected from H, and optionally substituted (C₁-C₆)alkyl. In certain cases. L¹ is —C₁₋₆-alkylene-, such as —C₁₋₃-alkylene-. In certain cases, L¹ is —(CH₂CH₂O)—, where t is 1 to 20, such as 1 to 15, 1 to 10, 1 to 8, 1 to 6, or 1 to 4. In certain cases, L¹ is —C₁₋₆-alkylene-NR⁴CO—. In certain cases, L¹ is —C₁₋₆-alkyleneCONH—. In certain cases, L¹ is or OCH₂.

In some embodiments of formula (IIb), one or more L¹ is independently —CH₂O—; —(CH₂CH₂O)ₜ—, —NR⁴CO—, —C₁₋₆-alkylene-,

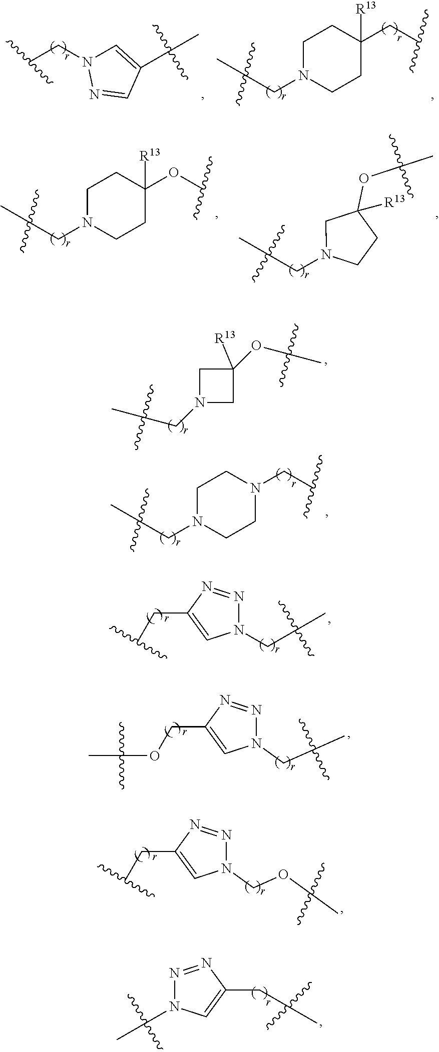

wherein: R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR²¹, —COOR²¹, —CONHR²¹, and —NHCOR²¹;
each r independently 0 to 20, and any of the L¹ moieties are optionally further substituted.

In certain embodiments of formula (IIb), L² is independently selected from:
—NR^{4′}CO—C₁₋₆-alkylene-, —CONR^{4′}—C₁₋₆-alkylene,

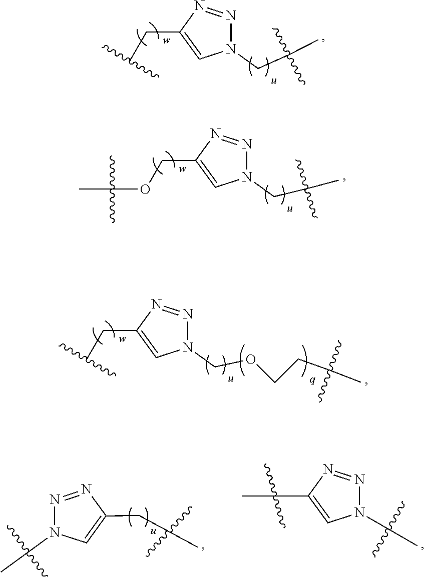

—OCH₂—, and —(OCH₂CH₂)_{q}—, wherein q is 1 to 10, u is 0 to 10, w is 1 to 10, and R^{4′} is independently selected from H. and optionally substituted (C₁-C₆)alkyl. In certain cases, L² is —NR^{4′}CO—C₁₋₆-alkylene-. In certain cases, L² is —CONR^{4′}—C₁₋₆-alkylene.

In certain cases, L² is

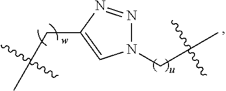

where w is 1 and u is 0 or 1.

In certain cases, L² is

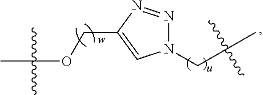

where w is 1 and u is 0 or 1.

In certain cases, L² is

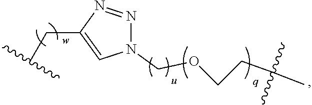

where w is 1, u is 0 or 1, and q is 1.

In certain cases, L² is

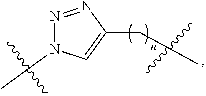

where u is 0 or 1.

In certain cases, L² is

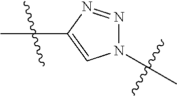 [SMILES: *c1cn(*)nn1]

In certain embodiments, L² is —OCH₂—. In certain other embodiments. L² is (OCH_{Z}CH_{Z})_{q}—, and q is 1 to 10, 1 to 9, 1 to 8, 1 to 7, 1 to 6, 1 to 5, 1 to 4, 1 to 3 or 1 to 2. In certain cases, q is 2 to 8, such as 2 to 6, 4 to 6, or 2 to 4.

In certain embodiments of formula (IIb), L⁴ is absent or independently selected from —C₁₋₆-alkylene-, —(CH₂CH₂O)ₜ—, —C₁₋₆-alkylene-NHCO—, —C₁₋₆-alkyleneCONH—, or OCH₂, wherein t is 1 to 20. In certain cases, L⁴ is absent. In certain cases. L⁴ is —C₁₋₆-alkylene-. In certain cases. L⁴ is —(CH₂CH₂O)ₜ—, where t is 1 to 20, such as 1 to 15, 1 to 12, 1 to 10, 1 to 8, 1 to 6, 1 to 4 or 1 to 3. In certain cases, L4 is —C₁₋₆-alkylene-NHCO—. In certain cases, L⁴ is —C₁₋₆-alkyleneCONH—. In certain cases, L⁴ is OCH₂.

In some embodiments of the subject compounds, n is 1 and L³ in formula (IIb) is absent.

In certain embodiments of the subject compounds, n is 2 or more, and L³ of formula (IIb) is a branched linking moiety.

Accordingly, in some embodiments of formula (IIb). L³ is a branched linking moiety, e.g., a divalent, or a trivalent linking moiety. For example, an L³ linking moiety can be of the one of the following general formula:

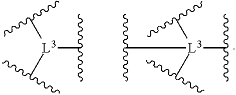

In some embodiments of formula (IIb), the branched linking moiety can be of higher valency and be described by one of the one of the following general formula:

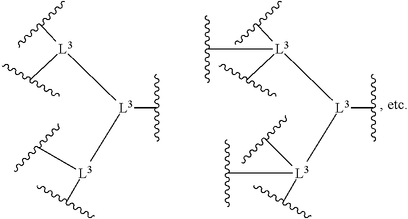

where any two L³ groups can be directed linked or connected via optional linear linking moieties (e.g., as described herein).

In some embodiments of formula (IIb), the branched linking moiety can include one, two or more L³ linking moieties, each being trivalent moieties, which when linked together can provide for multiple branching points for covalent attachment of the ligands and be described by the following general formula:

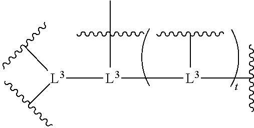

where t is 0 to 500, such as 0 to 100, 0 to 20, or 0 to 10.

In some embodiments, the branched linking moiety (e.g., L³) comprises one or more of: an amino acid residue (e.g., Asp, Lys, Orn, Glu), N-substituted amido (—N(−)C(═O)—), tertiary amino, polyol (e.g., O-substituted glycerol), and the like.

In some embodiments of formula (IIb), one or more L³ is a branching moiety selected from

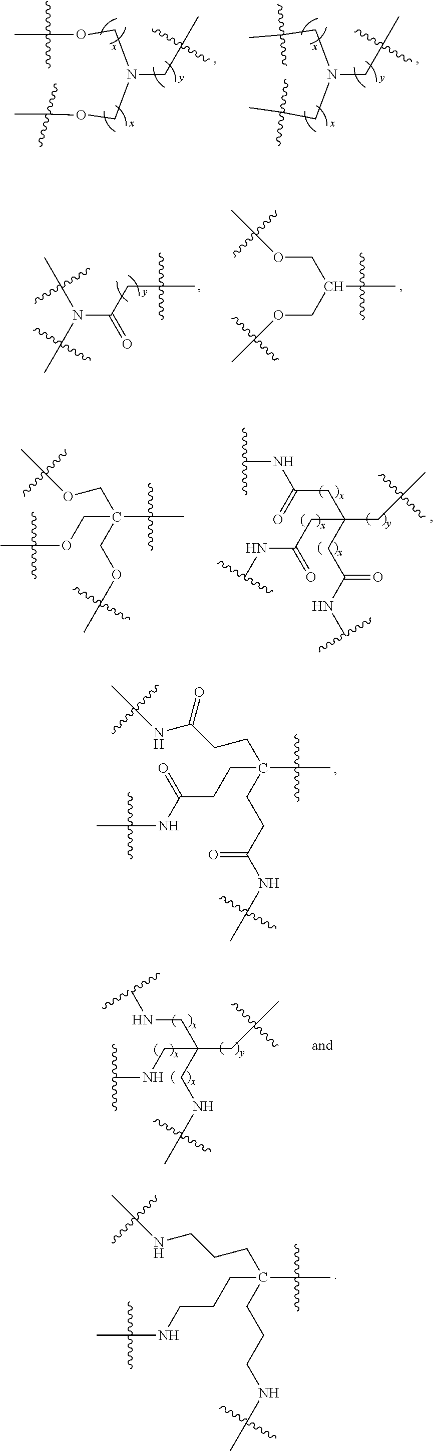

wherein each x and y are each independently 1 to 10, such as 1-6, 1-3, e.g., 1 or 2. In some cases, each x is 1, 2 or 3, e.g., 2.

In some embodiments of formula (IIb), L⁵ is selected from —CH₂O—; —(CH₂CH₂O)ₜ—, —NR⁴CO—, —C₁₋₆-alkylene-.

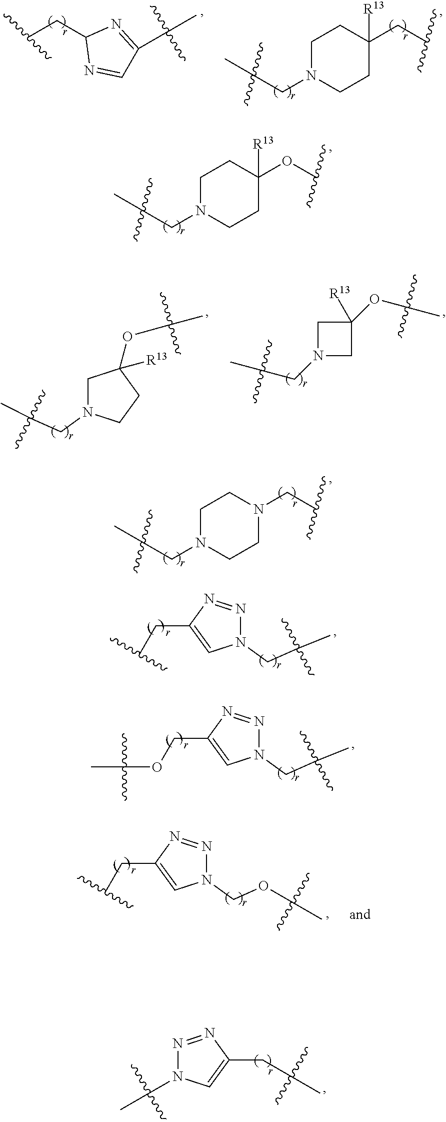

wherein:
R¹³ is selected from H, halogen, OH, optionally substituted (C₁-C₆)alkyl, optionally substituted (C₁-C₆)alkoxy, COOH, NO₂, CN, NH₂, —N(R²¹)₂, —OCOR^{2′}, —COOR²¹, —CONHR²¹, and —NHCOR²¹;
each r independently 0 to 20, and any of the L moieties are optionally further substituted.

In certain cases, L⁵ is —CH₂O—. In certain cases, L⁵ is —(CH₂CH₂O)ₜ—, where t is 1 to 20, such as 1-15, 1-12, 1-10, 1-8, 1-6, or 1 to 4. In certain cases. L⁵ is —NR⁴CO—, where R⁴ is H, or optionally substituted (C₁-C₆)alkyl. In certain cases. L⁵ is —C₁₋₆-alkylene-.

In certain cases, L⁵ is

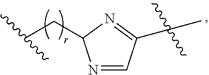

where r is 0 to 20, such as 0 to 15, 0 to 10, 0 to 8, or 0 to 5.

In certain cases, L⁵ is

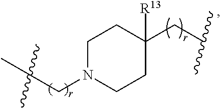

where each r is independently 0 to 20, such as 0 to 15, 0 to 10, 0 to 8, or 0 to 5 and R¹³ is H, or optionally substituted (C₁-C₆)alkyl.

In certain cases, L⁵ is

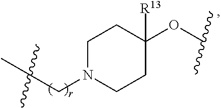

where r is 0 to 20, such as 0 to 15, 0 to 10, 0 to 8, or 0 to 5 and R¹³ is H, or optionally substituted (C₁-C₆)alkyl.

In certain cases, L⁵ is

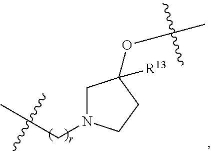

where r is 0 to 20, such as 0 to 15, 0 to 10, 0 to 8, or 0 to 5, and R¹³ is H, or optionally substituted (C₁-C₆)alkyl.

In certain cases, L⁵ is

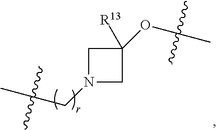

where r is 0 to 20, such as 0 to 15, 0 to 10, 0 to 8, or 0 to 5, and R¹³ is H, or optionally substituted (C₁-C₆)alkyl.

In certain cases, L⁵ is

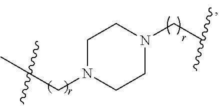

where each r is independently 0 to 20, such as 0 to 15, 0 to 10, 0 to 8, or 0 to 5.

In certain cases, L⁵ is

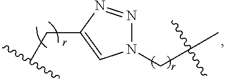

where each r is independently 0 to 20, such as 0 to 15, 0 to 10, 0 to 8, or 0 to 5.

In certain cases, L⁵ is

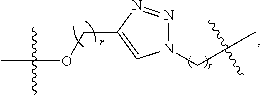

where each r is independently 0 to 20, such as 0 to 15, 0 to 10, 0 to 8, or 0 to 5.

In certain cases, L⁵ is

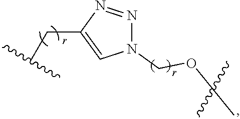

where each r is independently 0 to 20, such as 0 to 15, 0 to 10, 0 to 8, or 0 to 5.

In certain cases, L⁵ is

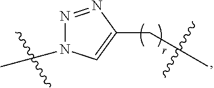

where r is 0 to 20, such as 0 to 15, 0 to 10, 0 to 8, or 0 to 5.

In some embodiments of formula (IIb), L⁵ comprises one or more of: an amino acid residue (e.g., Asp, Lys, Orn, Glu), an amino acid analogue, N-substituted amido (—N(−)C(═O)—), tertiary amino, polyol (e.g., 0-substituted glycerol), and the like. Analogs of an amino acid, include but not limited to, unnatural amino acids, as well as other modifications known in the art. The amino acid includes L-amino acids. D-amino acids, or both, and may contain any of a variety of amino acid modifications or analogs known in the art.

In some embodiments of formula (IIb), L¹-L⁵ comprises one or more of the following units:

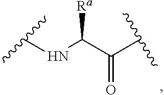

where R^{a} is (C₁-C₆)alkyl or substituted (C₁-C₆)alkyl, e.g., a (C₁-C₆)alkyl optionally substituted with amine, a tertiary amine, optionally substituted alkoxy, optionally substituted carboxyl, optionally substituted aryl, or optionally substituted heteroaryl. It is understood that R^{a} can be linked to a M6PR binding moiety.

In some embodiments, the linker includes a polypeptide scaffold where some or all of the sidechain groups of the amino acid residues have been modified to attach a X binding moiety (e.g., as described herein). It is understood that X binding moieties (e.g., as described herein) can be conjugated to amino acid residues, such as Asp. Lys, Orn, Glu, and Ser, of a polypeptide containing linker via a convenient conjugation chemistry. In some embodiments, the linker contains a polylysine polypeptide. In some embodiments, the linker contains a polyornithine polypeptide. In some embodiments, the linker contains a polyserine polypeptide. In some embodiments, the linker contains a polyaspartate polypeptide. The polypeptide can be a randomly polymerized polymer having an average length, or a polymer of defined length prepared e.g., in a controlled stepwise fashion. In some cases, the polypeptide linker segment has a length of 10-100 amino acid residues, such as 20-90, or 20-50 amino acid residues. In some embodiments, the N-terminal or C-terminal of the polypeptide linker segment is modified to include a linking unit to an additional M6PR binding moiety (e.g., as described herein). In some embodiments, the N-terminal or C-terminal of the polypeptide linker segment is modified with one or more linking units (e.g., as described herein) suitable for attachment to a Y moiety of interest.

In certain embodiments of formula (IIb), a is 1. In certain cases, at least one of b, c, d, and e is not 0. In certain cases, b is 1 or 2. In certain cases, c is 1 or 2. In certain cases, e is 1 or 2. In certain cases, b, d and e are independently 1 or 2. In certain cases, a, b, d, and e are each 1, and c is 0.

In certain embodiments of formula (II), (IIa) or (IIb), the linker comprises 20 to 100 consecutive atoms, such as 20 to 90, 20 to 80, 20 to 70, 20 to 60, 20 to 50, 20 to 40 or 20 to 30 consecutive atoms. In certain cases, the linker comprises 25 to 100 consecutive atoms, such as 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive atoms.

In certain embodiments of formula (II), (IIa) or (IIb), the linker comprises 25 or more consecutive atoms, such as 26 or more, 27 or more, 28 or more, 29 or more or 30 or more consecutive atoms. In certain embodiments of formula (II), (IIa) or (IIb), the linker comprises 30 or more consecutive atoms, such as 31 or more, 32 or more, 33 or more, 34 or more, 35 or more, 36 or more, 37, or more, 38 or more, 39 or more, 40 or even more consecutive atoms.

The inventors have demonstrated that compounds of this disclosure having a particular configuration with a linker of desired valency and length can specifically bind with high affinity to both the receptor and a target simultaneously, and exhibit high uptake activity of a target. The conjugates of this disclosure can thus provide for sequestering of a target protein in the cell's lysosome and degrading of the target protein.

### Exemplary Linkers and Linking Moieties

Exemplary linkers and linking moieties that can be utilized in the preparation of compounds of this disclosure (e.g., that link the ASGPR ligand (X) to the moiety of interest (Y)) are shown in Tables 6-8.

In certain embodiments, the linker is a linear linker or linking moiety as shown in Table 6.

TABLE 6
Exemplary linear linkers and linking moieties

| Linker No. | Linker structure |
|---|---|
| L1 | 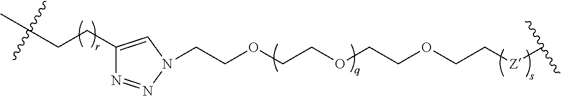 |
| | r is 0 to 10, q is 0 to 20, s is 0 or 1, Z′ is CO, NHCO, CONH or NH |
| L1.1 | 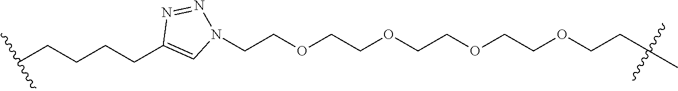 [SMILES: *CCCCc1cn(CCOCCOCCOCCOCC*)nn1] |
| L1.2 | 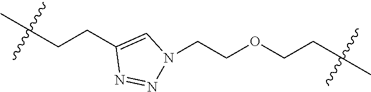 [SMILES: *CCOCCn1cc(CC*)nn1] |
| L1.3 | 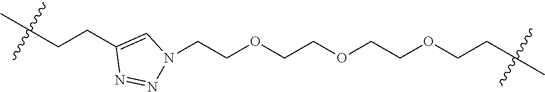 [SMILES: *CCOCCOCCOCCn1cc(CC*)nn1] |
| L1.4 | 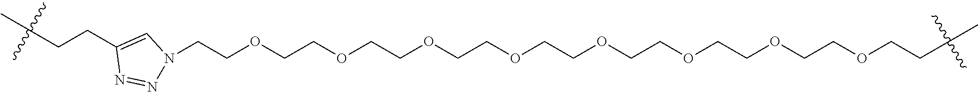 [SMILES: *CCOCCOCCOCCOCCOCCOCCOCCOCCn1cc(CC*)nn1] |
| L1.5 | 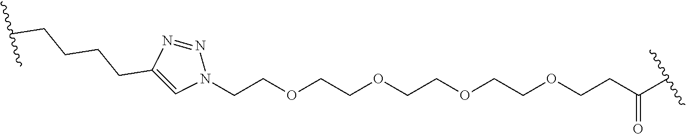 [SMILES: *CCCCc1cn(CCOCCOCCOCCOCCC(*)=O)nn1] |
| L1.6 | 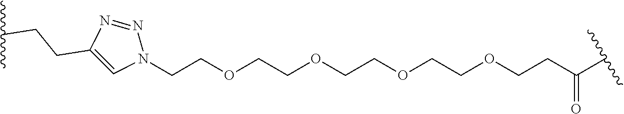 [SMILES: *CCc1cn(CCOCCOCCOCCOCCC(*)=O)nn1] |
| L1.7 | 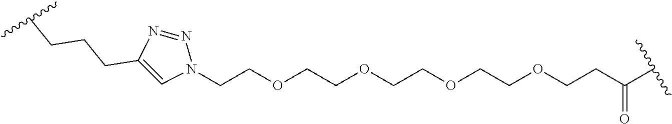 [SMILES: *CCCc1cn(CCOCCOCCOCCOCCC(*)=O)nn1] |
| L1.8 | 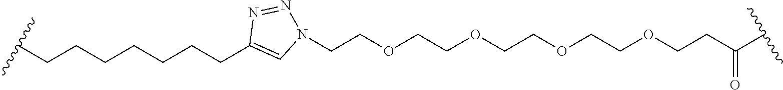 [SMILES: *CCCCCCCc1cn(CCOCCOCCOCCOCCC(*)=O)nn1] |
| L1.9 | 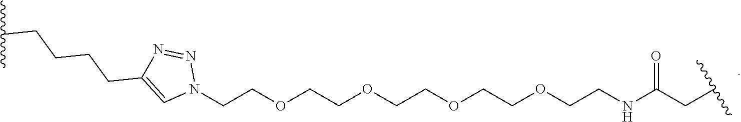 [SMILES: *CCCCc1cn(CCOCCOCCOCCOCCNC(=O)C*)nn1] |
| L1.10 | 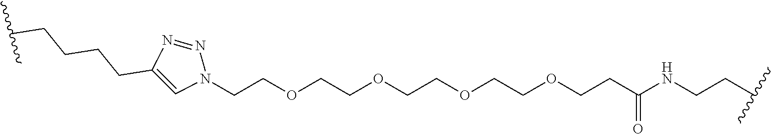 [SMILES: *CCCCc1cn(CCOCCOCCOCCOCCC(=O)NCC*)nn1] |
| L1.11 | 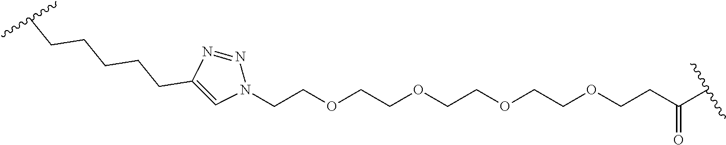 [SMILES: *CCCCCc1cn(CCOCCOCCOCCOCCC(*)=O)nn1] |
| L2 | 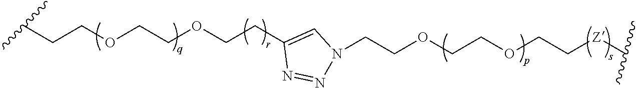 |
| | r is 0 to 10, p and q are 0 to 20, s is 0 or 1, Z′ is CO, NHCO, CONH or NH |
| L2.1 | 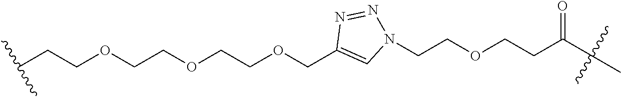 [SMILES: *CCOCCOCCOCc1cn(CCOCCC(*)=O)nn1] |
| L3 | 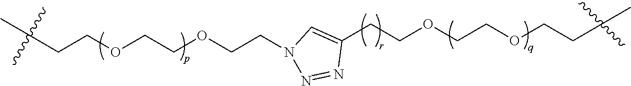 |
| | r is 0 to 10, p and q are independently 0 to 20 |
| L4 | 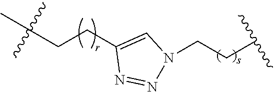 |
| | r is 0 to 10, s is 1 to 10 |
| L5 | 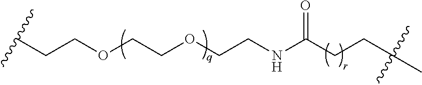 |
| | or |
| | 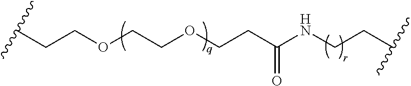 |
| | where r is 0 to 10, q is 0 to 20 |
| L5.1 | 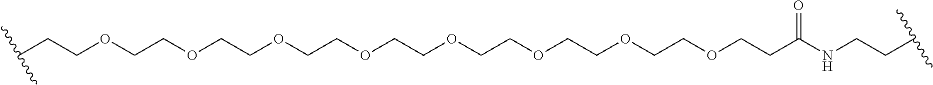 [SMILES: *CCNC(=O)CCOCCOCCOCCOCCOCCOCCOCCOCC*] |
| L6 | 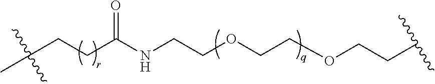 |
| | or |
| | 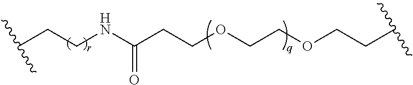 |
| | r is 0 to 10, q is 0 to 20 |
| L7 | 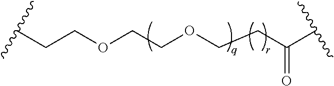 |
| | r is 0 to 10, q is 0 to 20 |
| L7.1 | 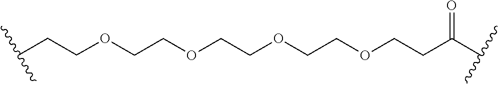 [SMILES: *CCOCCOCCOCCOCCC(*)=O] |
| L7.2 | 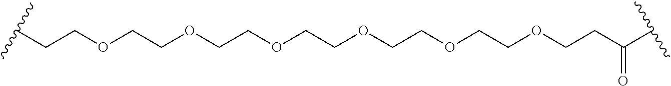 [SMILES: *CCOCCOCCOCCOCCOCCOCCC(*)=O] |
| L7.3 | 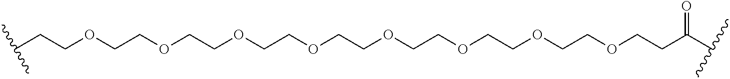 [SMILES: *CCOCCOCCOCCOCCOCCOCCOCCOCCC(*)=O] |
| L8 | 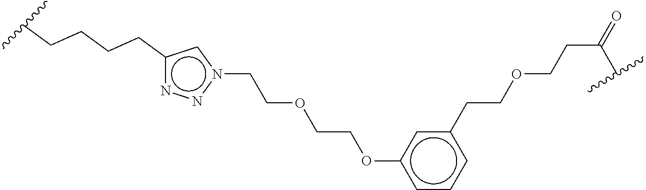 |
| L9 | 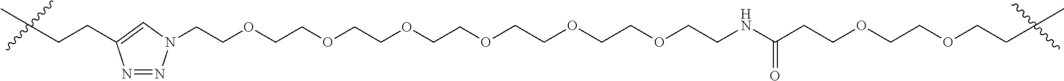 [SMILES: *CCOCCOCCC(=O)NCCOCCOCCOCCOCCOCCOCCn1cc(CC*)nn1] |
| L10 | 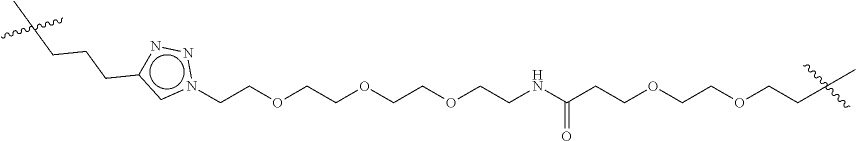 |
| L11 | 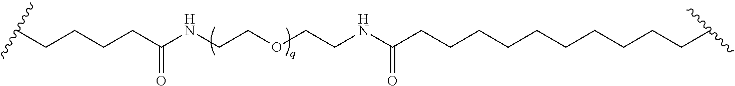 |
| | q is 0 to 10 |
| L12 | 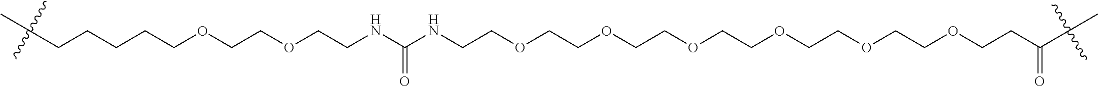 [SMILES: *CCCCCOCCOCCNC(=O)NCCOCCOCCOCCOCCOCCOCCC(*)=O] |
| L13 | 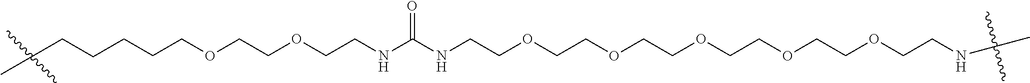 [SMILES: *CCCCCOCCOCCNC(=O)NCCOCCOCCOCCOCCOCCN*] |

Table 7 includes various linker component synthetic precursors (e.g., linear and branched linker precursors) that can be utilized in the preparation of the subject compounds.

TABLE 7
Linker component synthetic precursors

| Reagent # | Structure |
|---|---|
| LC1 | 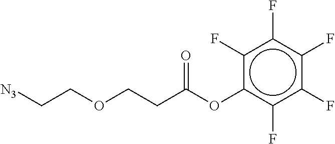 |
| LC1.1 | 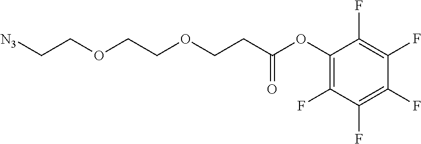 [SMILES: [N-]=[N+]=NCCOCCOCCC(=O)Oc1c(F)c(F)c(F)c(F)c1F] |
| LC2 | 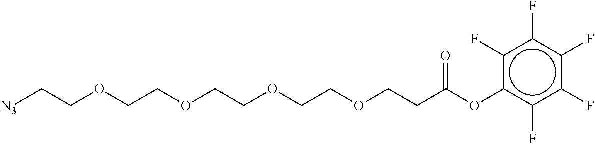 |
| LC3 | 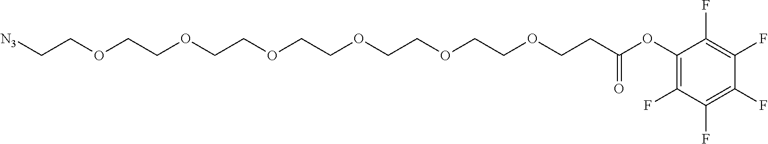 [SMILES: [N-]=[N+]=NCCOCCOCCOCCOCCOCCOCCC(=O)Oc1c(F)c(F)c(F)c(F)c1F] |
| LC3.1 | 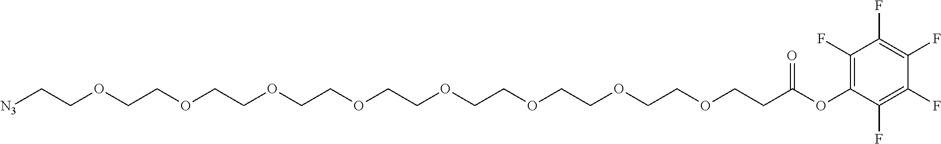 [SMILES: [N-]=[N+]=NCCOCCOCCOCCOCCOCCOCCOCCOCCC(=O)Oc1c(F)c(F)c(F)c(F)c1F] |
| LC3.2 | 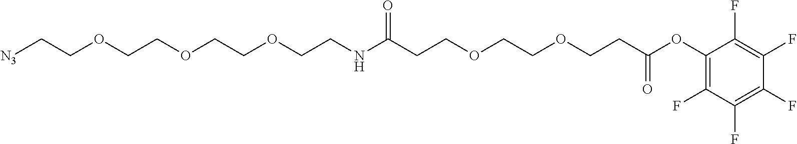 [SMILES: [N-]=[N+]=NCCOCCOCCOCCNC(=O)CCOCCOCCC(=O)Oc1c(F)c(F)c(F)c(F)c1F] |
| LC4 | 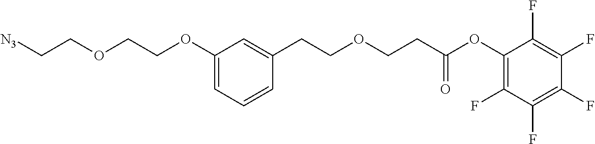 [SMILES: [N-]=[N+]=NCCOCCOc1cccc(CCOCCC(=O)Oc2c(F)c(F)c(F)c(F)c2F)c1] |
| LC5 | 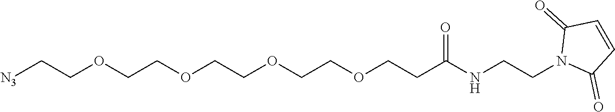 [SMILES: [N-]=[N+]=NCCOCCOCCOCCOCCC(=O)NCCN1C(=O)C=CC1=O] |
| LC6 | 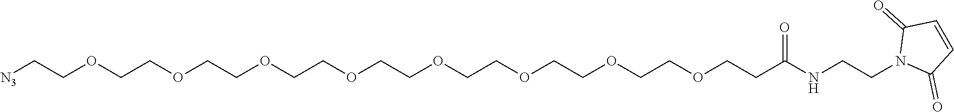 [SMILES: [N-]=[N+]=NCCOCCOCCOCCOCCOCCOCCOCCOCCC(=O)NCCN1C(=O)C=CC1=O] |
| LC7 | 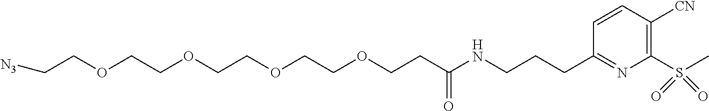 [SMILES: CS(=O)(=O)c1nc(CCCNC(=O)CCOCCOCCOCCOCCN=[N+]=[N-])ccc1C#N] |
| LC8 | 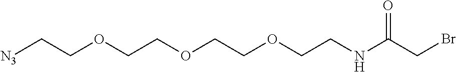 [SMILES: [N-]=[N+]=NCCOCCOCCOCCNC(=O)CBr] |
| LC9 | 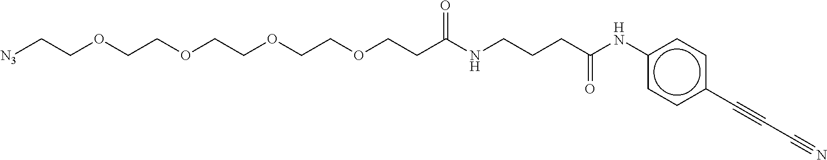 |
| LC9.1 | 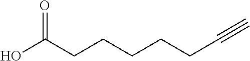 |
| LC10 | 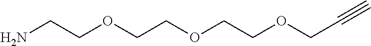 |
| LC10.1 | 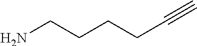 |
| LC10.2 | 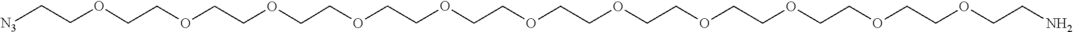 [SMILES: [N-]=[N+]=NCCOCCOCCOCCOCCOCCOCCOCCOCCOCCOCCOCCN] |
| LC10.3 | 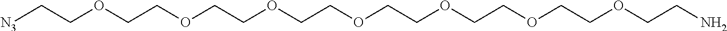 [SMILES: [N-]=[N+]=NCCOCCOCCOCCOCCOCCOCCOCCN] |
| LC10.4 | 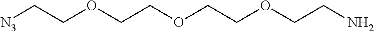 [SMILES: [N-]=[N+]=NCCOCCOCCOCCN] |
| LC10.5 | 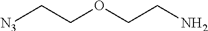 |
| LC10.6 | 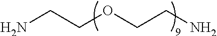 |
| LC10.7 | 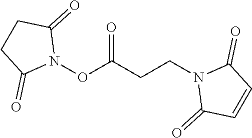 [SMILES: O=C(CCN1C(=O)C=CC1=O)ON1C(=O)CCC1=O] |
| LC10.8 | 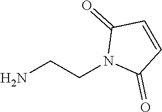 |
| LC11 | 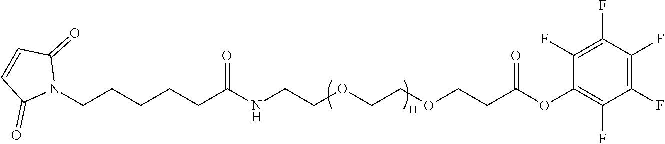 |
| LC12 | 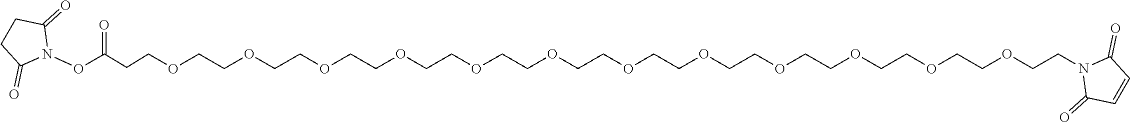 [SMILES: O=C(CCOCCOCCOCCOCCOCCOCCOCCOCCOCCOCCOCCOCCN1C(=O)C=CC1=O)ON1C(=O)CCC1=O] |
| LC13 | 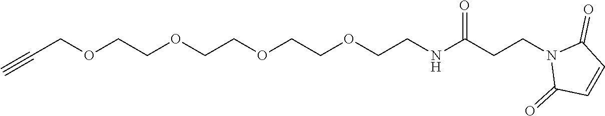 [SMILES: C#CCOCCOCCOCCOCCNC(=O)CCN1C(=O)C=CC1=O] |
| LC14 | 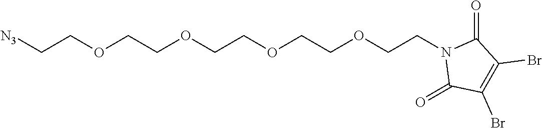 [SMILES: [N-]=[N+]=NCCOCCOCCOCCOCCN1C(=O)C(Br)=C(Br)C1=O] |
| LC15 | 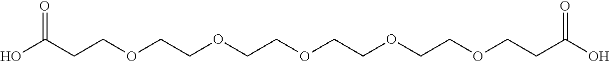 [SMILES: O=C(O)CCOCCOCCOCCOCCOCCC(=O)O] |
| LC16 | 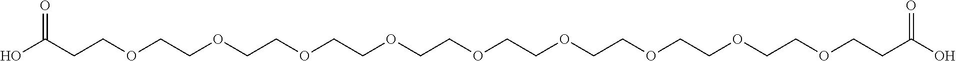 [SMILES: O=C(O)CCOCCOCCOCCOCCOCCOCCOCCOCCOCCC(=O)O] |
| LC17(1-3) | 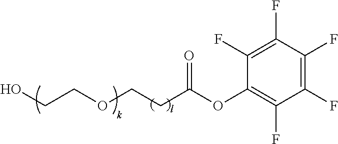 |
| | k = 4, 1 = 0 |
| | k = 0, 1 = 12 |
| | k = 2, 1 = 6 |
| LC18 | 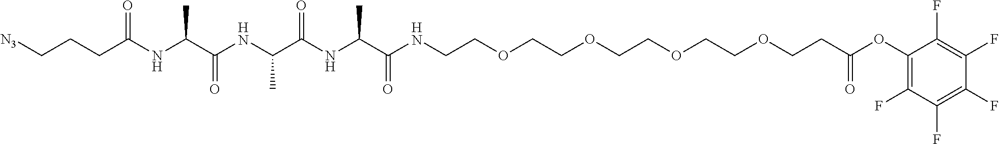 [SMILES: C[C@H](NC(=O)CCCN=[N+]=[N-])C(=O)N[C@@H](C)C(=O)N[C@@H](C)C(=O)NCCOCCOCCOCCOCCC(=O)Oc1c(F)c(F)c(F)c(F)c1F] |
| LC19 | 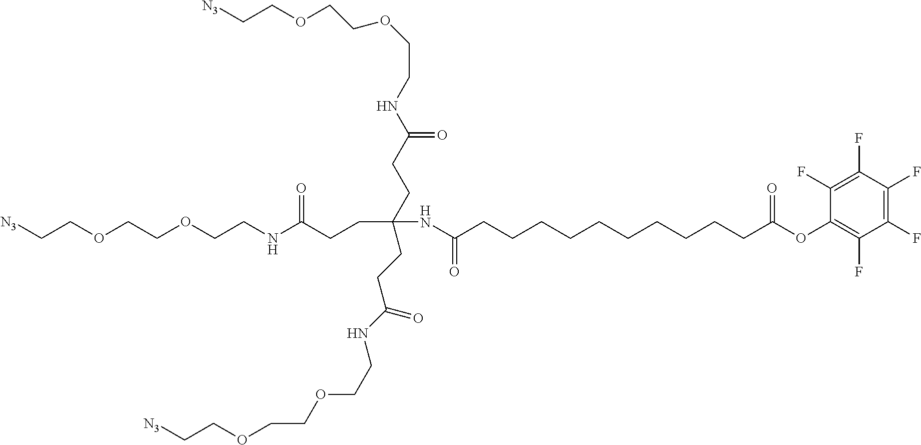 [SMILES: [N-]=[N+]=NCCOCCOCCNC(=O)CCC(CCC(=O)NCCOCCOCCN=[N+]=[N-])(CCC(=O)NCCOCCOCCN=[N+]=[N-])NC(=O)CCCCCCCCCCC(=O)Oc1c(F)c(F)c(F)c(F)c1F] |
| LC19.1 | 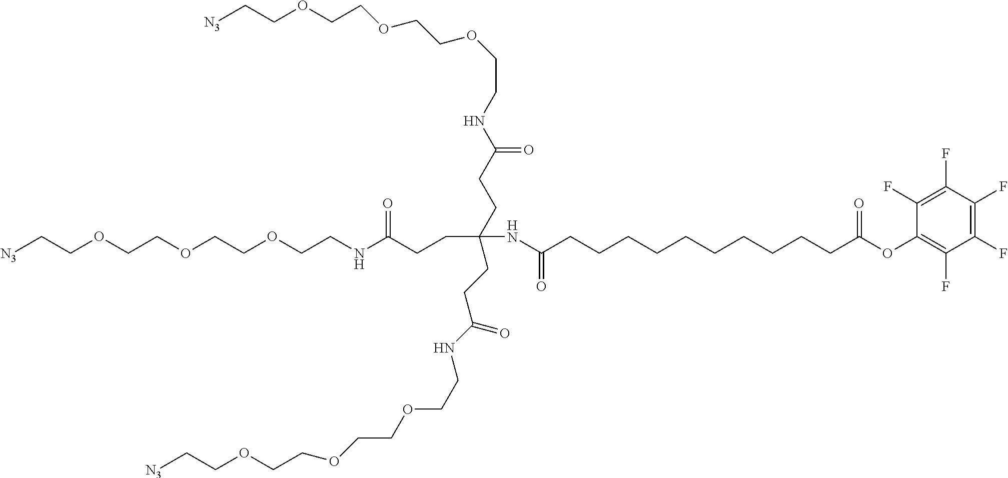 [SMILES: [N-]=[N+]=NCCOCCOCCOCCNC(=O)CCC(CCC(=O)NCCOCCOCCOCCN=[N+]=[N-])(CCC(=O)NCCOCCOCCOCCN=[N+]=[N-])NC(=O)CCCCCCCCCCC(=O)Oc1c(F)c(F)c(F)c(F)c1F] |
| LC20 | 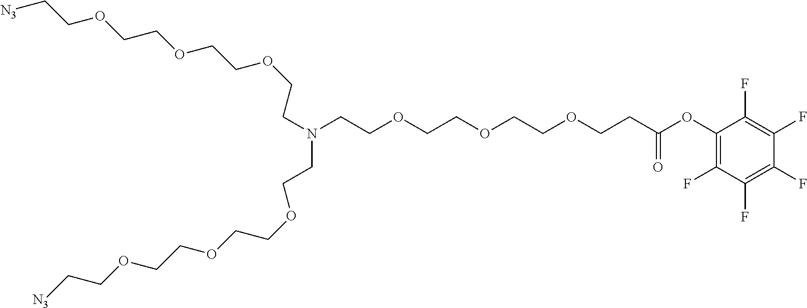 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F] |
| LC21 | 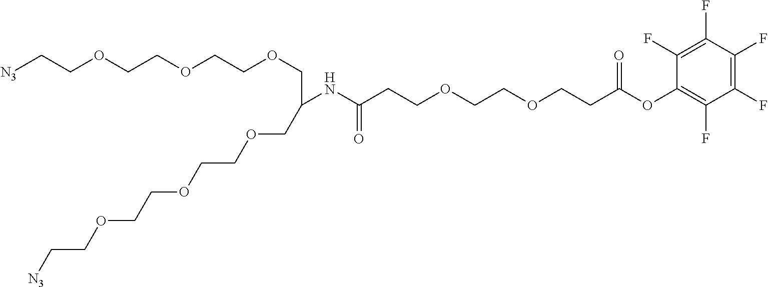 [SMILES: [N-]=[N+]=NCCOCCOCCOCC(COCCOCCOCCN=[N+]=[N-])NC(=O)CCOCCOCCC(=O)Oc1c(F)c(F)c(F)c(F)c1F] |
| LC22 | 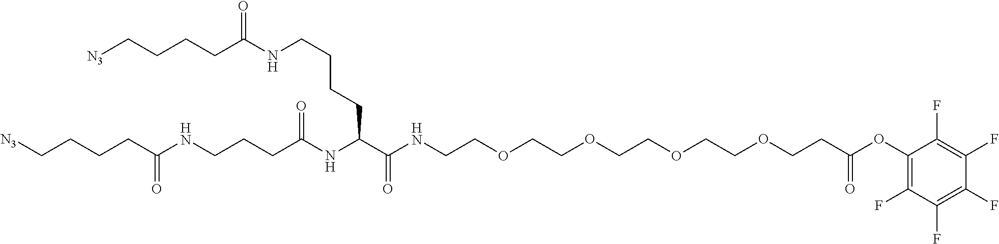 [SMILES: [N-]=[N+]=NCCCCC(=O)NCCCC[C@H](NC(=O)CCCNC(=O)CCCCN=[N+]=[N-])C(=O)NCCOCCOCCOCCOCCC(=O)Oc1c(F)c(F)c(F)c(F)c1F] |
| LC23 | 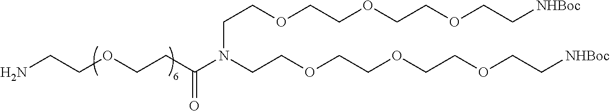 |
| LC24 | 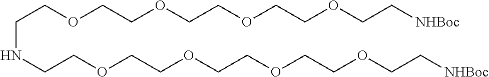 [SMILES: CC(C)(C)OC(=O)NCCOCCOCCOCCOCCNCCOCCOCCOCCOCCNC(=O)OC(C)(C)C] |
| LC25 | 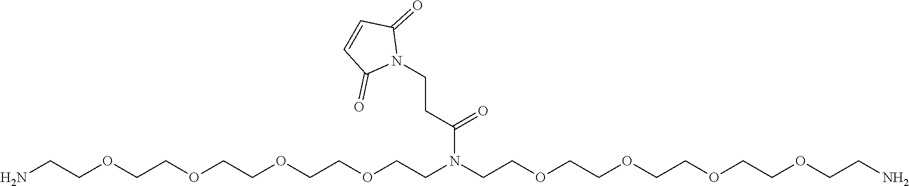 [SMILES: NCCOCCOCCOCCOCCN(CCOCCOCCOCCOCCN)C(=O)CCN1C(=O)C=CC1=O] |
| LC26 | 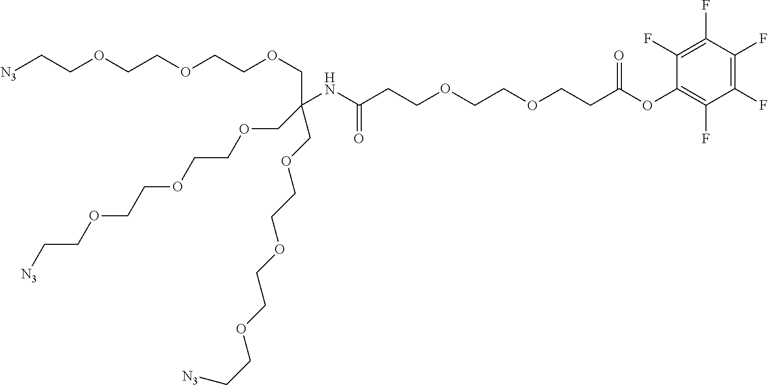 [SMILES: [N-]=[N+]=NCCOCCOCCOCC(COCCOCCOCCN=[N+]=[N-])(COCCOCCOCCN=[N+]=[N-])NC(=O)CCOCCOCCC(=O)Oc1c(F)c(F)c(F)c(F)c1F] |
| LC27 | 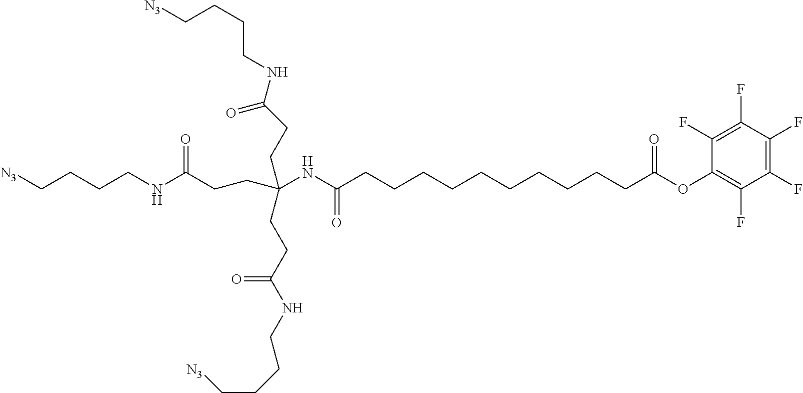 [SMILES: [N-]=[N+]=NCCCCNC(=O)CCC(CCC(=O)NCCCCN=[N+]=[N-])(CCC(=O)NCCCCN=[N+]=[N-])NC(=O)CCCCCCCCCCC(=O)Oc1c(F)c(F)c(F)c(F)c1F] |
| LC28 | 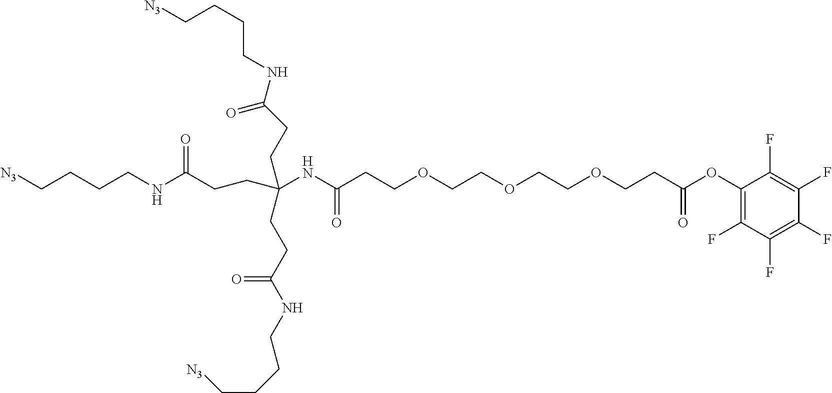 [SMILES: [N-]=[N+]=NCCCCNC(=O)CCC(CCC(=O)NCCCCN=[N+]=[N-])(CCC(=O)NCCCCN=[N+]=[N-])NC(=O)CCOCCOCCOCCC(=O)Oc1c(F)c(F)c(F)c(F)c1F] |
| LC29 | 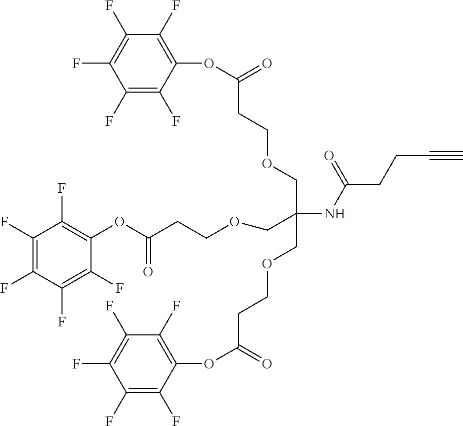 [SMILES: C#CCCC(=O)NC(COCCC(=O)Oc1c(F)c(F)c(F)c(F)c1F)(COCCC(=O)Oc1c(F)c(F)c(F)c(F)c1F)COCCC(=O)Oc1c(F)c(F)c(F)c(F)c1F] |
| LC30 | 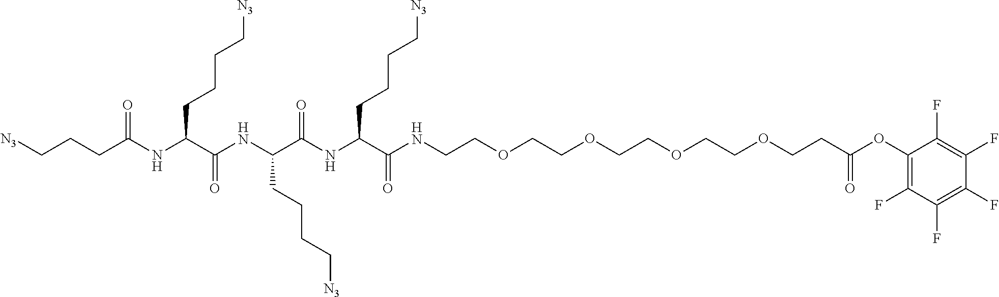 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC31 | 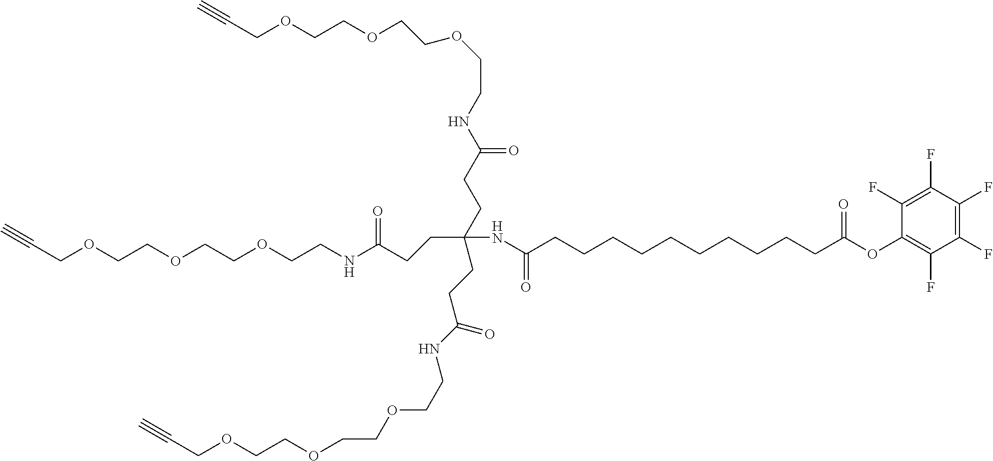 [SMILES: C#CCOCCOCCOCCNC(=O)CCC(CCC(=O)NCCOCCOCCOCC#C)(CCC(=O)NCCOCCOCCOCC#C)NC(=O)CCCCCCCCCCC(=O)Oc1c(F)c(F)c(F)c(F)c1F] |
| LC32 | 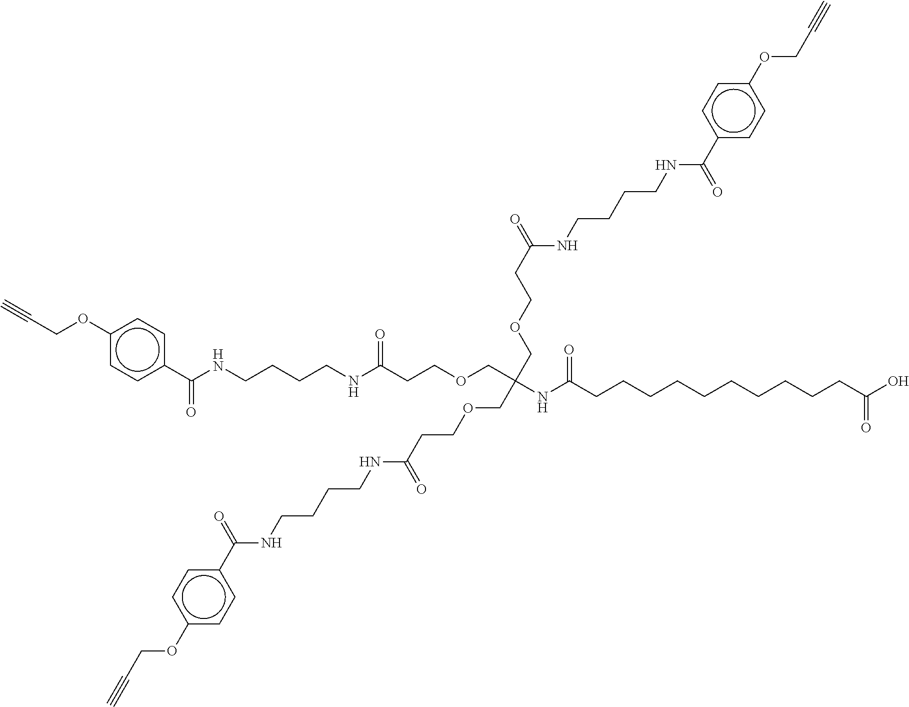 |
| LC33 | 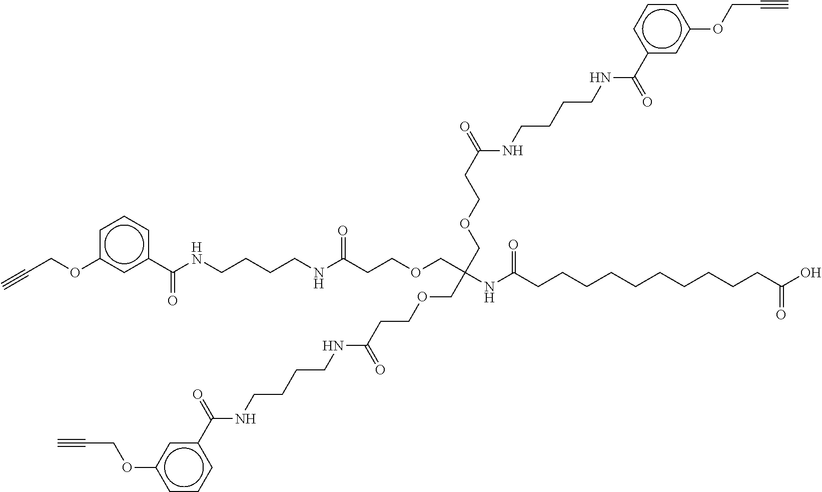 |
| LC34 | 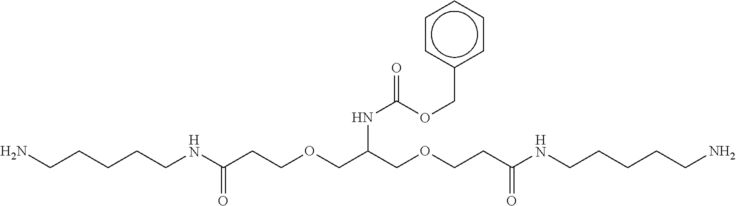 |
| LC35 | 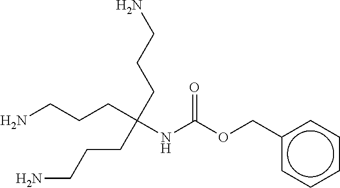 |
| LC36 | 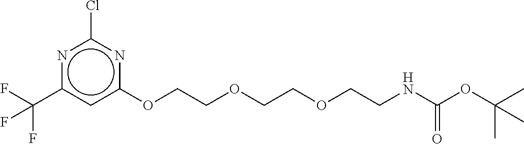 |
| LC37 | 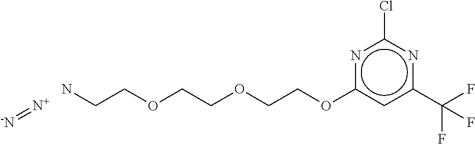 |

In certain embodiments, the linker is a branched linker or linking moiety as shown in Table 8.

TABLE 8
Exemplary branched linkers and branched linking moieties

| Linker | |
|---|---|
| No. | Linker structure |
| L21 | 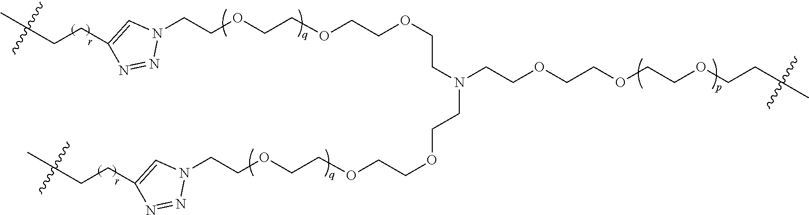 |
| | r is 0 to 10, q and p are independently 0 to 20 |
| L22 | 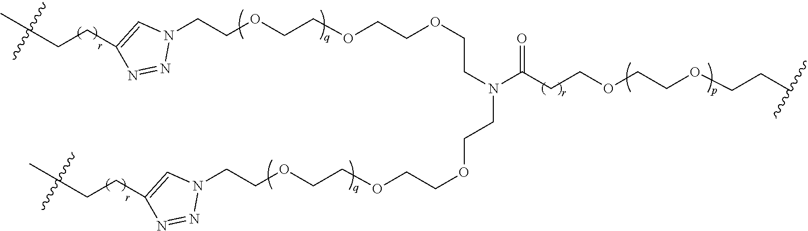 |
| | each r is independently 0 to 10, q and p are independently 0 to 20 |
| L23 | 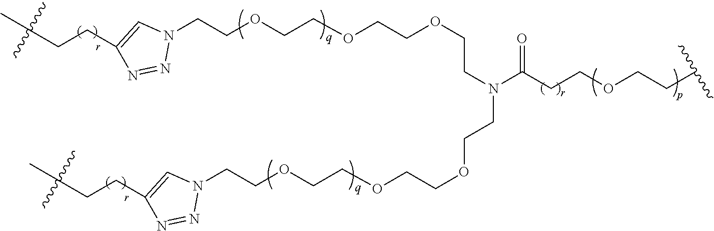 |
| | each r is independently 0 to 10, q and p are independently 0 to 20 |
| L24 | 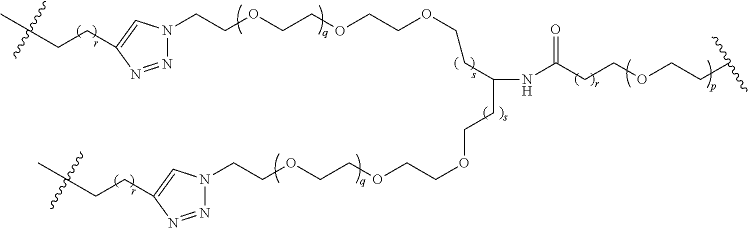 |
| | each r is independently 0 to 10, s is 0 or 1, q and p are independently 0 to 20 |
| L25 | 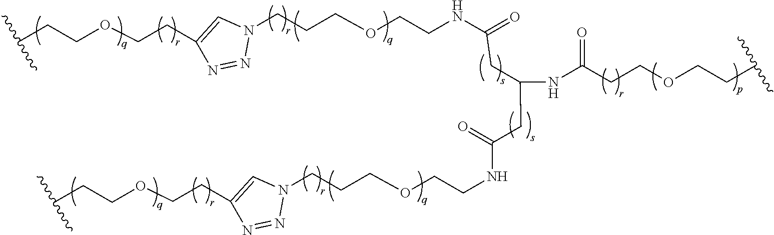 |
| | each r is independently 0 to 10, s is 0 or 1, each q and p is independently 0 to 20 |
| L26 | 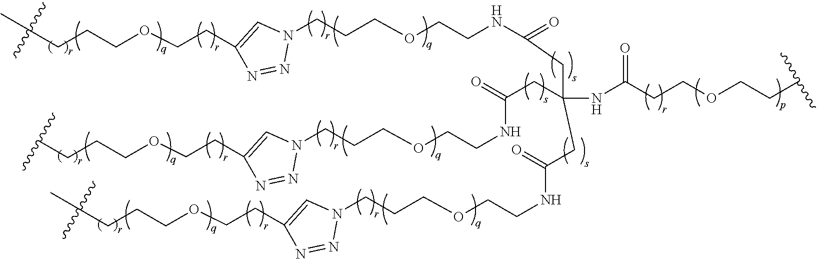 |
| | each r is independently 0 to 10, s is 0 or 1, each q and p is independently 0 to 20 |
| L26.1 | 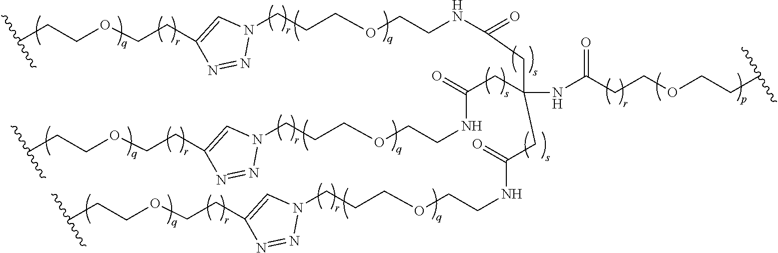 |
| | each r is independently 0 to 10, s is 0 or 1, each q and p is independently 0 to 20 |
| L27 | 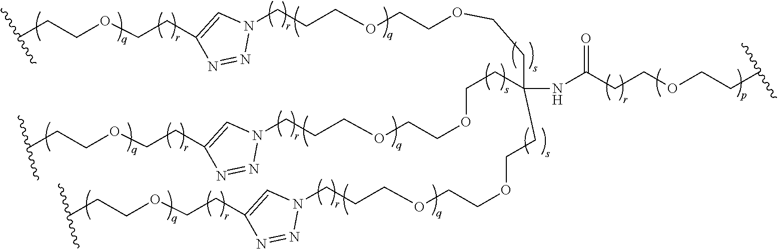 |
| | each r is independently 0 to 10, s is 0 or 1, each q and p is independently 0 to 20 |
| L28 | 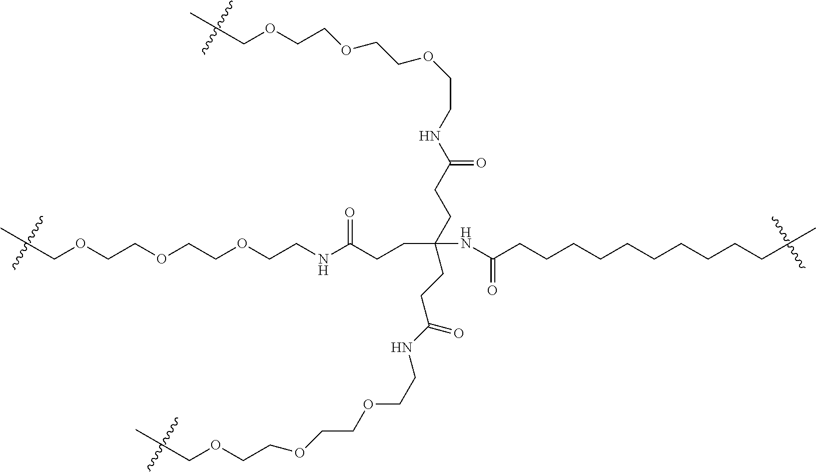 [SMILES: *CCCCCCCCCCC(=O)NC(CCC(=O)NCCOCCOCCOC*)(CCC(=O)NCCOCCOCCOC*)CCC(=O)NCCOCCOCCOC*] |
| L29 | 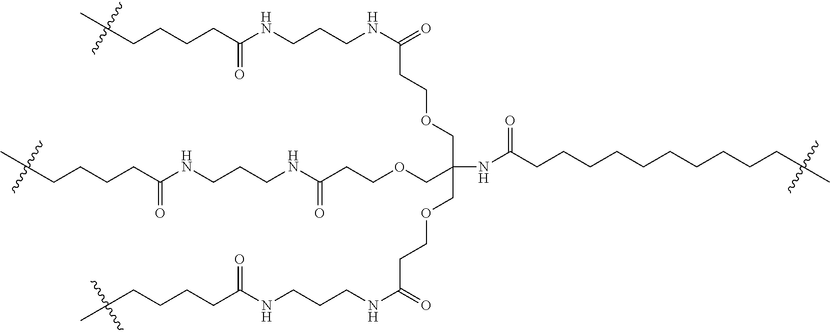 [SMILES: *CCCCCCCCCCC(=O)NC(COCCC(=O)NCCCNC(=O)CCCC*)(COCCC(=O)NCCCNC(=O)CCCC*)COCCC(=O)NCCCNC(=O)CCCC*] |
| L30 | 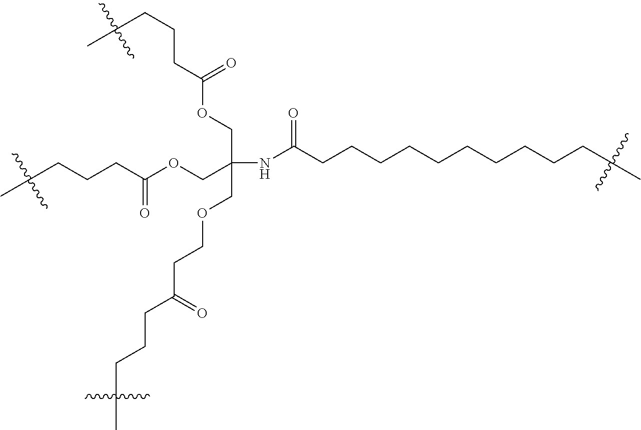 [SMILES: *CCCCCCCCCCC(=O)NC(COCCC(=O)CCC*)(COC(=O)CCC*)COC(=O)CCC*] |
| L31 | 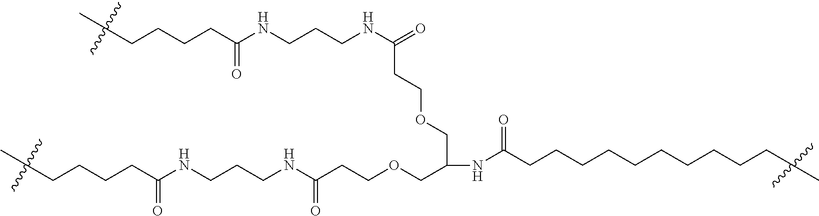 [SMILES: *CCCCCCCCCCC(=O)NC(COCCC(=O)NCCCNC(=O)CCCC*)COCCC(=O)NCCCNC(=O)CCCC*] |
| L32 | 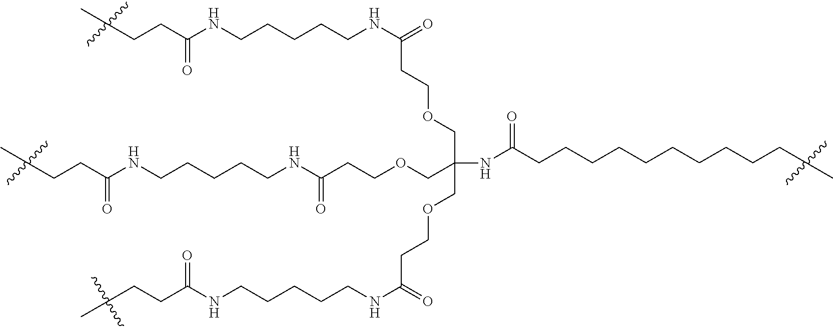 [SMILES: *CCCCCCCCCCC(=O)NC(COCCC(=O)NCCCCCNC(=O)CC*)(COCCC(=O)NCCCCCNC(=O)CC*)COCCC(=O)NCCCCCNC(=O)CC*] |
| L33 | 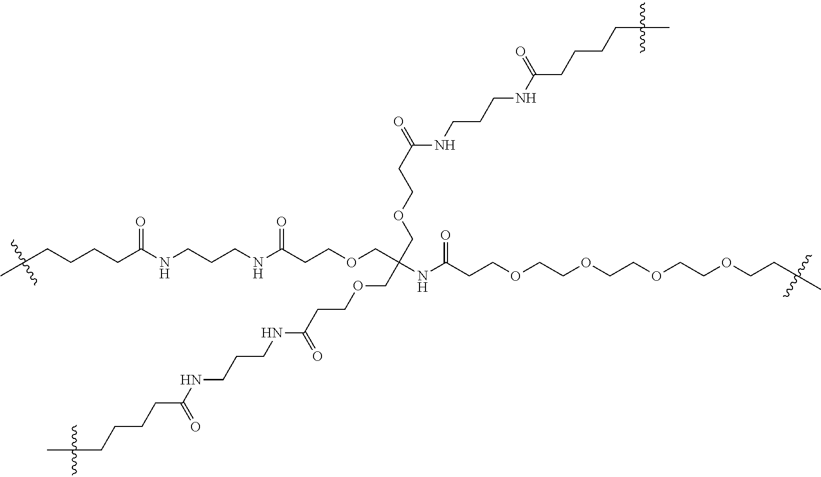 [SMILES: *CCCCC(=O)NCCCNC(=O)CCOCC(COCCC(=O)NCCCNC(=O)CCCC*)(COCCC(=O)NCCCNC(=O)CCCC*)NC(=O)CCOCCOCCOCCOCC*] |
| L34 | 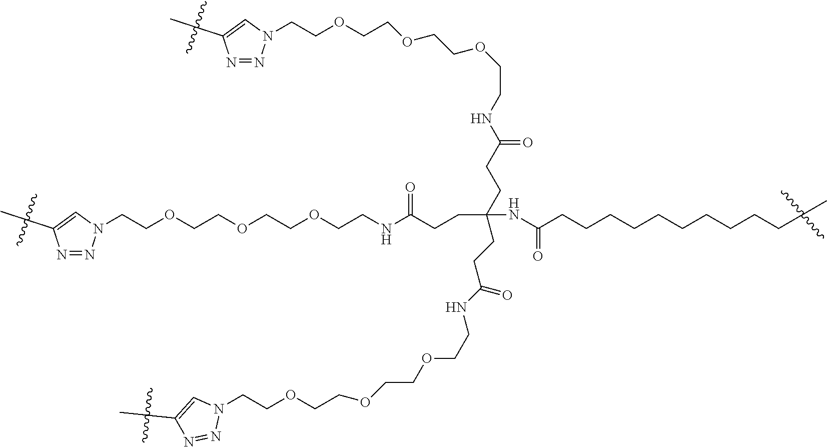 [SMILES: *CCCCCCCCCCC(=O)NC(CCC(=O)NCCOCCOCCOCCn1cc(*)nn1)(CCC(=O)NCCOCCOCCOCCn1cc(*)nn1)CCC(=O)NCCOCCOCCOCCn1cc(*)nn1] |
| L35 L36 | 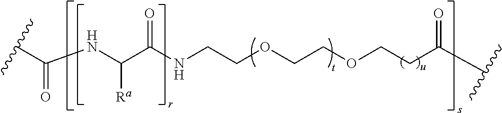 |
| | 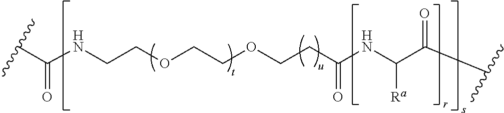 |
| | where r is 1-3, t is 3-5, u is 0 or 1, and s is 2-5 |

Table 9 illustrates exemplary synthetic precursors of linker components that are used to prepare compounds of this disclosure. e.g., via a conjugation chemistry. It is understood that a variety of homologs of the structures shown in Table 9 are also encompassed by this disclosure that provide for linkers of a variety of lengths. It is understood that alternative chemoselective ligation groups and other chemical functional groups can also be incorporated as needed to prepare a desired linker.

TABLE 9
Linker component synthetic precursors

| Reagent | |
|---|---|
| # | Structure |
| LC1 | 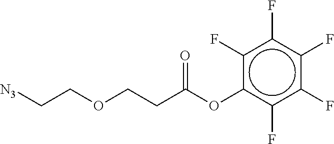 |
| LC2 | 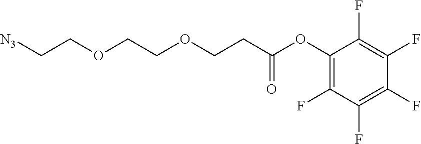 [SMILES: [N-]=[N+]=NCCOCCOCCC(=O)Oc1c(F)c(F)c(F)c(F)c1F] |
| LC3 | 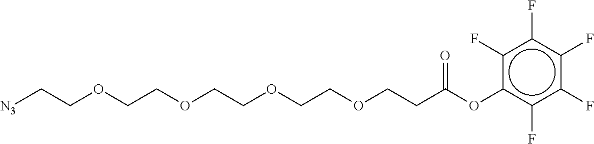 |
| LC4 | 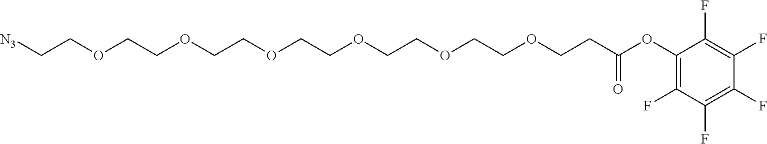 [SMILES: [N-]=[N+]=NCCOCCOCCOCCOCCOCCOCCC(=O)Oc1c(F)c(F)c(F)c(F)c1F] |
| LC5 | 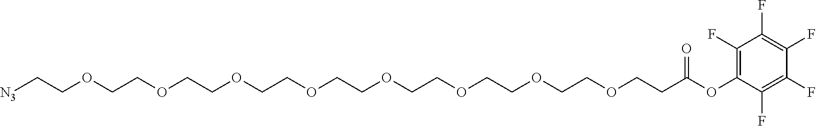 [SMILES: [N-]=[N+]=NCCOCCOCCOCCOCCOCCOCCOCCOCCC(=O)Oc1c(F)c(F)c(F)c(F)c1F] |
| LC6 | 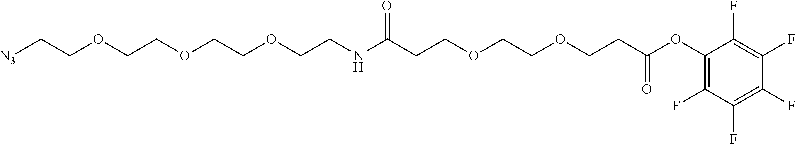 [SMILES: [N-]=[N+]=NCCOCCOCCOCCNC(=O)CCOCCOCCC(=O)Oc1c(F)c(F)c(F)c(F)c1F] |
| LC7 | 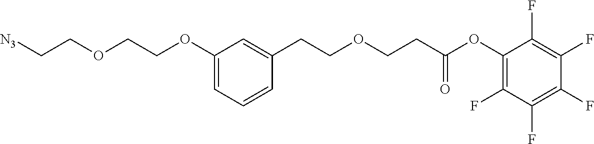 [SMILES: [N-]=[N+]=NCCOCCOc1cccc(CCOCCC(=O)Oc2c(F)c(F)c(F)c(F)c2F)c1] |
| LC8 | 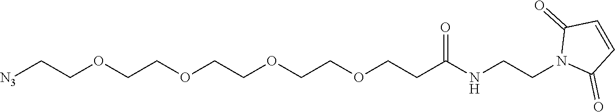 [SMILES: [N-]=[N+]=NCCOCCOCCOCCOCCC(=O)NCCN1C(=O)C=CC1=O] |
| LC9 | 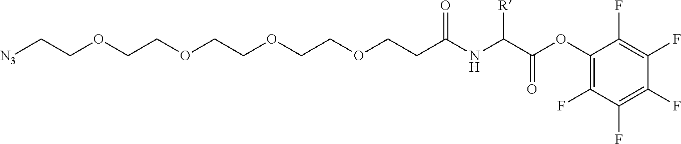 |
| LC10 | 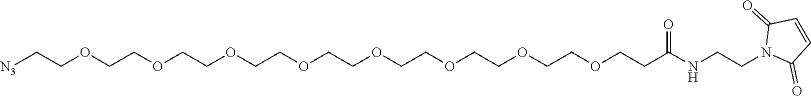 [SMILES: [N-]=[N+]=NCCOCCOCCOCCOCCOCCOCCOCCOCCC(=O)NCCN1C(=O)C=CC1=O] |
| LC11 | 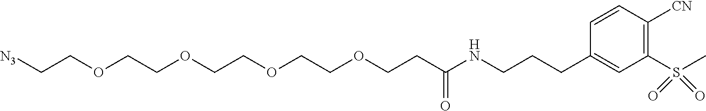 [SMILES: CS(=O)(=O)c1cc(CCCNC(=O)CCOCCOCCOCCOCCN=[N+]=[N-])ccc1C#N] |
| LC12 | 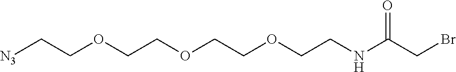 [SMILES: [N-]=[N+]=NCCOCCOCCOCCNC(=O)CBr] |
| LC13 | 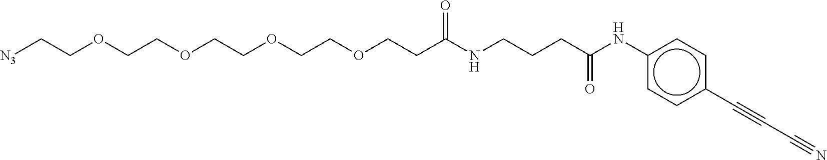 |
| LC14 | 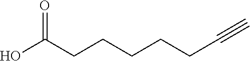 |
| LC15 | 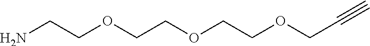 |
| LC16 | 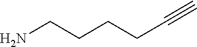 |
| LC17 | 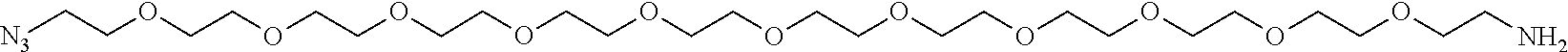 [SMILES: [N-]=[N+]=NCCOCCOCCOCCOCCOCCOCCOCCOCCOCCOCCOCCN] |
| LC18 | 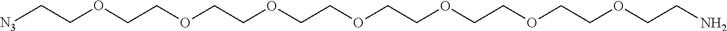 [SMILES: [N-]=[N+]=NCCOCCOCCOCCOCCOCCOCCOCCN] |
| LC19 | 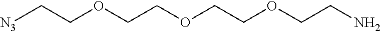 [SMILES: [N-]=[N+]=NCCOCCOCCOCCN] |
| LC20 | 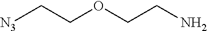 |
| LC21 | 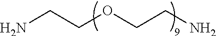 |
| LC22 | 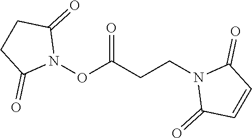 [SMILES: O=C(CCN1C(=O)C=CC1=O)ON1C(=O)CCC1=O] |
| LC23 | 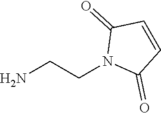 |
| LC24 | 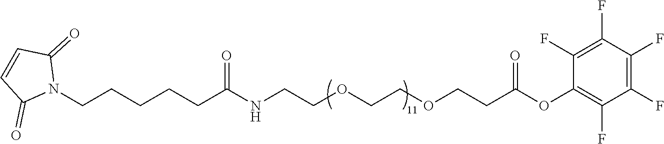 |
| LC25 | 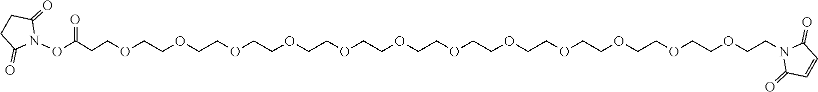 [SMILES: O=C(CCOCCOCCOCCOCCOCCOCCOCCOCCOCCOCCOCCOCCN1C(=O)C=CC1=O)ON1C(=O)CCC1=O] |
| LC26 | 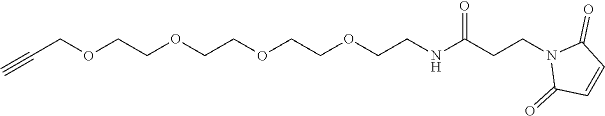 [SMILES: C#CCOCCOCCOCCOCCNC(=O)CCN1C(=O)C=CC1=O] |
| LC27 | 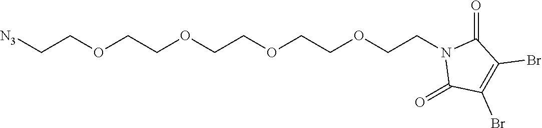 [SMILES: [N-]=[N+]=NCCOCCOCCOCCOCCN1C(=O)C(Br)=C(Br)C1=O] |
| LC28 | 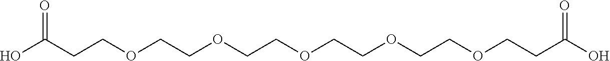 [SMILES: O=C(O)CCOCCOCCOCCOCCOCCC(=O)O] |
| LC29 | 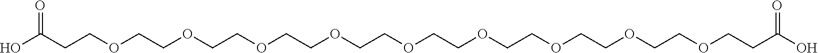 [SMILES: O=C(O)CCOCCOCCOCCOCCOCCOCCOCCOCCOCCC(=O)O] |
| LC30 | 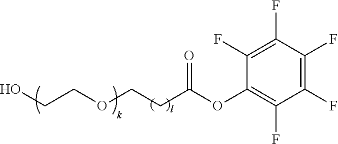 |
| | k = 4, l = 0 |
| | k = 0, l = 12 |
| | k = 2, l = 6 |
| LC31 | 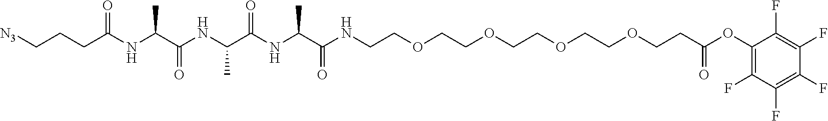 [SMILES: C[C@H](NC(=O)CCCN=[N+]=[N-])C(=O)N[C@@H](C)C(=O)N[C@@H](C)C(=O)NCCOCCOCCOCCOCCC(=O)Oc1c(F)c(F)c(F)c(F)c1F] |
| LC32 | 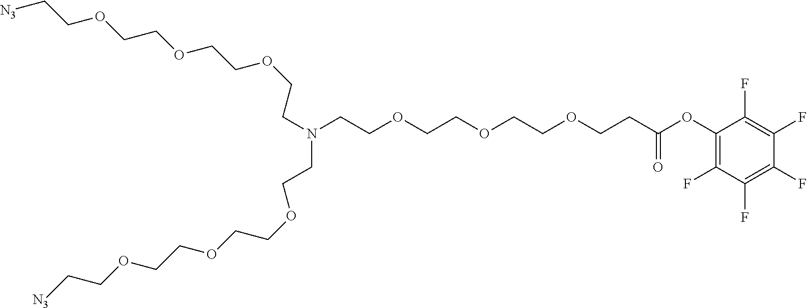 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F] |
| LC33 | 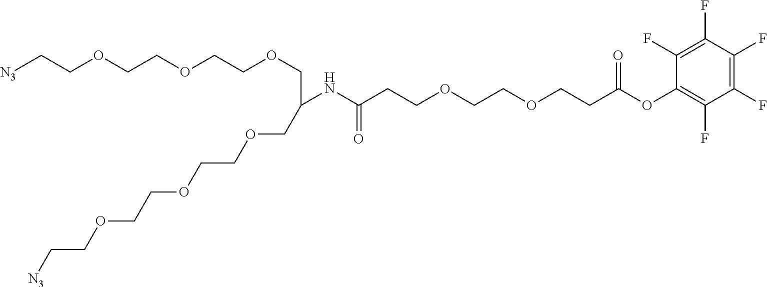 [SMILES: [N-]=[N+]=NCCOCCOCCOCC(COCCOCCOCCN=[N+]=[N-])NC(=O)CCOCCOCCC(=O)Oc1c(F)c(F)c(F)c(F)c1F] |
| LC34 | 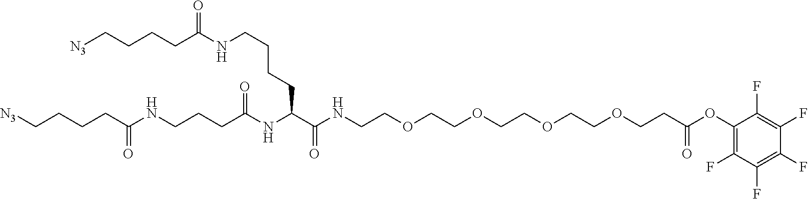 [SMILES: [N-]=[N+]=NCCCCC(=O)NCCCC[C@H](NC(=O)CCCNC(=O)CCCCN=[N+]=[N-])C(=O)NCCOCCOCCOCCOCCC(=O)Oc1c(F)c(F)c(F)c(F)c1F] |
| LC35 | 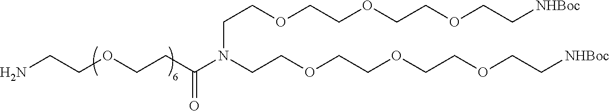 |
| LC36 | 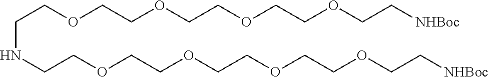 [SMILES: CC(C)(C)OC(=O)NCCOCCOCCOCCOCCNCCOCCOCCOCCOCCNC(=O)OC(C)(C)C] |
| LC37 | 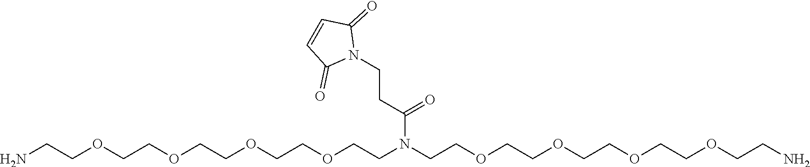 [SMILES: NCCOCCOCCOCCOCCN(CCOCCOCCOCCOCCN)C(=O)CCN1C(=O)C=CC1=O] |
| LC38 | 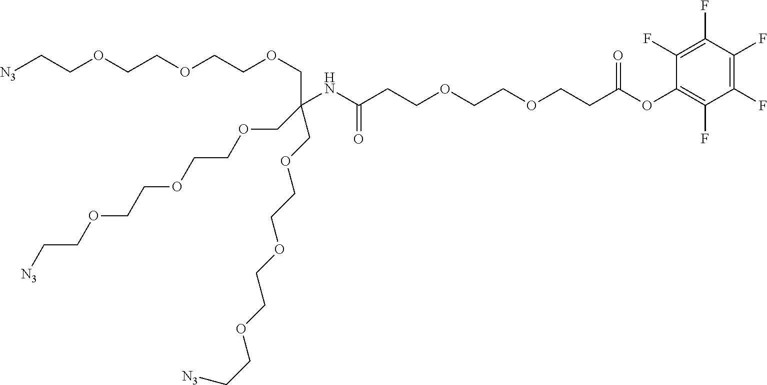 [SMILES: [N-]=[N+]=NCCOCCOCCOCC(COCCOCCOCCN=[N+]=[N-])(COCCOCCOCCN=[N+]=[N-])NC(=O)CCOCCOCCC(=O)Oc1c(F)c(F)c(F)c(F)c1F] |
| LC39 | 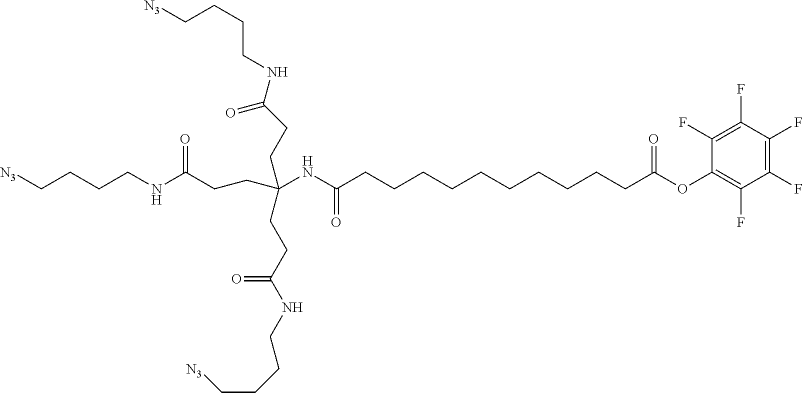 [SMILES: [N-]=[N+]=NCCCCNC(=O)CCC(CCC(=O)NCCCCN=[N+]=[N-])(CCC(=O)NCCCCN=[N+]=[N-])NC(=O)CCCCCCCCCCC(=O)Oc1c(F)c(F)c(F)c(F)c1F] |
| LC40 | 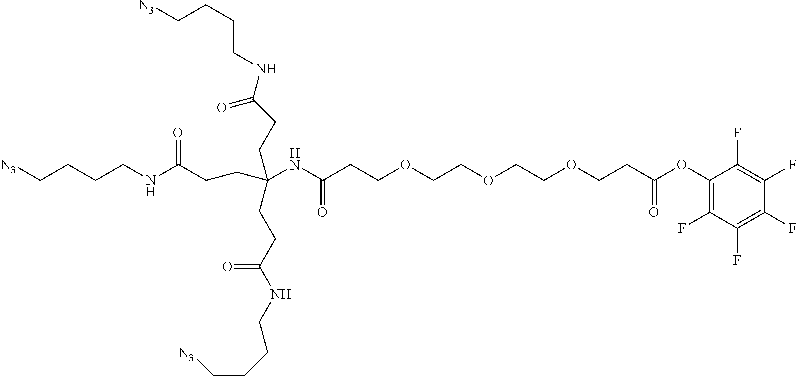 [SMILES: [N-]=[N+]=NCCCCNC(=O)CCC(CCC(=O)NCCCCN=[N+]=[N-])(CCC(=O)NCCCCN=[N+]=[N-])NC(=O)CCOCCOCCOCCC(=O)Oc1c(F)c(F)c(F)c(F)c1F] |
| LC41 | 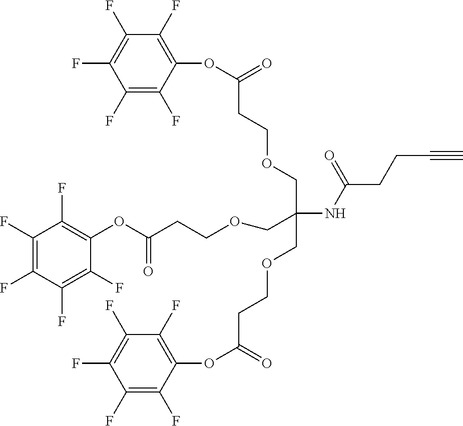 [SMILES: C#CCCC(=O)NC(COCCC(=O)Oc1c(F)c(F)c(F)c(F)c1F)(COCCC(=O)Oc1c(F)c(F)c(F)c(F)c1F)COCCC(=O)Oc1c(F)c(F)c(F)c(F)c1F] |
| LC42 | 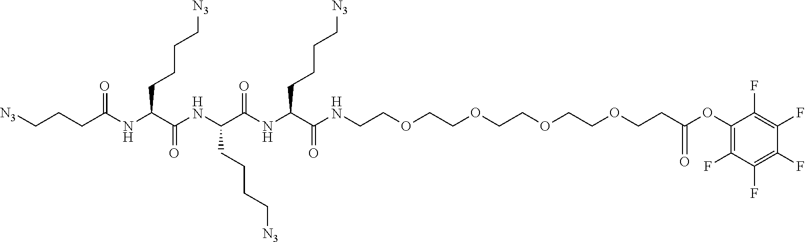 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC43 | 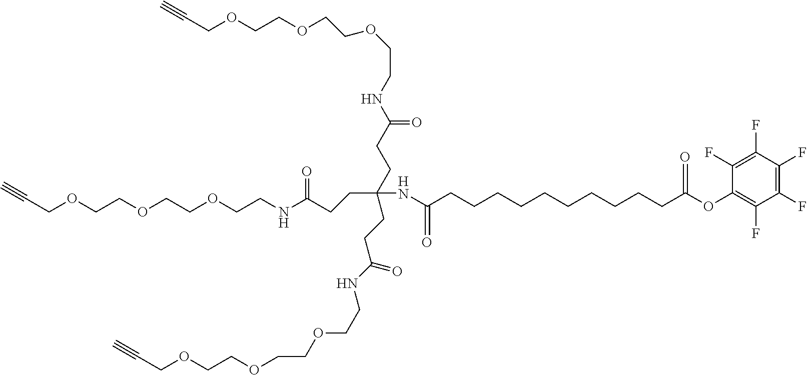 [SMILES: C#CCOCCOCCOCCNC(=O)CCC(CCC(=O)NCCOCCOCCOCC#C)(CCC(=O)NCCOCCOCCOCC#C)NC(=O)CCCCCCCCCCC(=O)Oc1c(F)c(F)c(F)c(F)c1F] |
| LC44 | 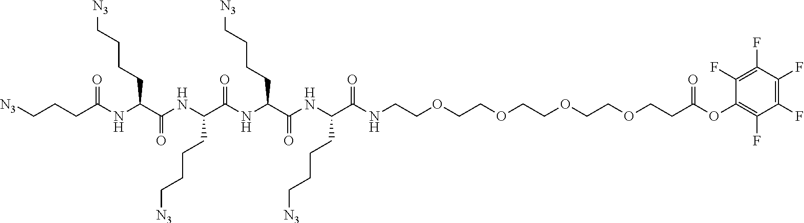 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| | where R is H, or protecting group, and s is 1, 2, 3, 5-10, or 10-100, or 20-50. |
| LC45 | 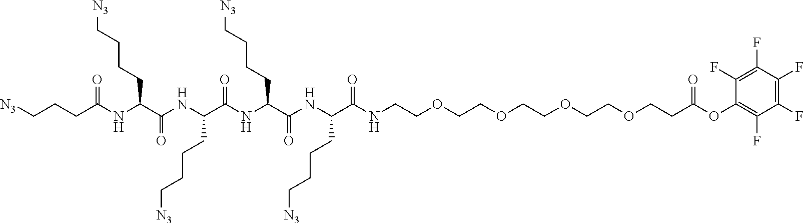 [SMILES: [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC46 | 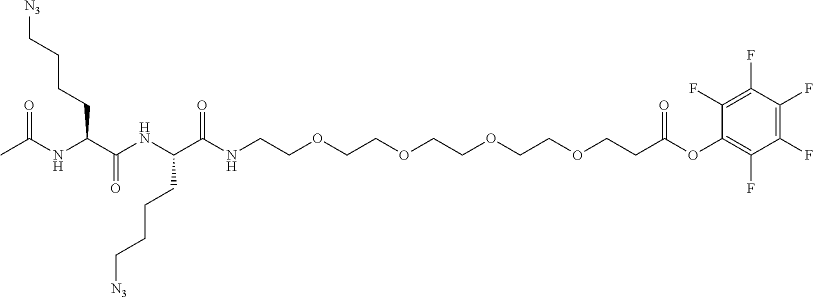 [SMILES: CC(=O)N[C@@H](CCCCN=[N+]=[N-])C(=O)N[C@@H](CCCCN=[N+]=[N-])C(=O)NCCOCCOCCOCCOCCC(=O)Oc1c(F)c(F)c(F)c(F)c1F] |
| LC47 | 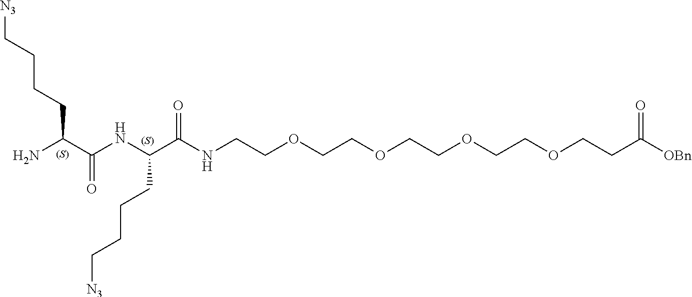 |
| LC48 | 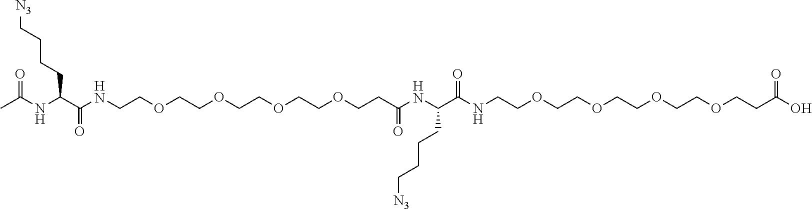 [SMILES: CC(=O)N[C@@H](CCCCN=[N+]=[N-])C(=O)NCCOCCOCCOCCOCCC(=O)N[C@@H](CCCCN=[N+]=[N-])C(=O)NCCOCCOCCOCCOCCC(=O)O] |
| LC49 LC50 LC51 LC52 LC53 | 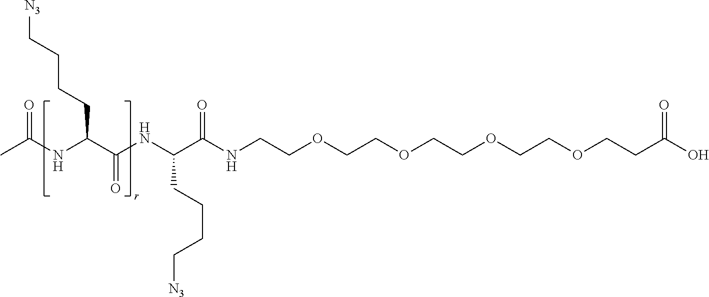 |
| | LC49 where r is 0 |
| | LC50 where r is 1 |
| | LC51 where r is 2 |
| | LC52 where r is 3 |
| | LC53 where r is 4 |
| LC54 LC55 LC56 LC57 LC58 LC59 LC60 | 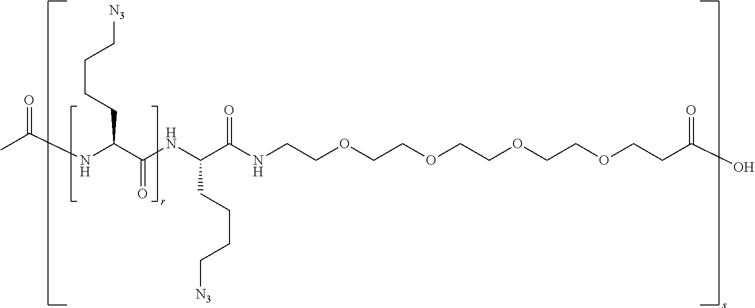 |
| | LC54 where r is 0, s is 2 |
| | LC55 where r is 1, s is 2 |
| | LC56 where r is 2, s is 2 |
| | LC57 where r is 0, s is 3 |
| | LC58 where r is 1, s is 3 |
| | LC59 where r is 2, s is 3 |
| | LC60 where r is 0-4, s is 4-20 |
| L61 L62 | 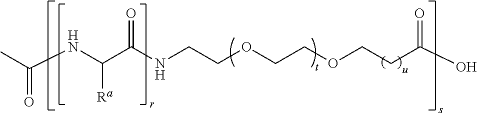 |
| | 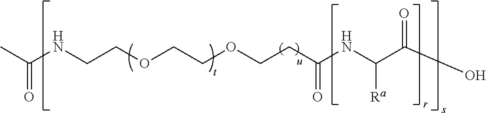 |
| | where r is 1-3, t is 3-5, u is 0 or 1, and s is 2-5 |

### Chemoselective Ligation Group

A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, tyrosine specific conjugation chemistry (e.g., e-Y-CLICK), methionine specific conjugation chemistry (e.g., oxaziridine-based or ReACT chemistry), reductive amination, dialkyl squarate chemistry, etc.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

In some instances, chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances, Y is a precursor of the reactive functionality or function group capable of conjugation to a compatible group of a second moiety. For example, a carboxylic acid is a precursor of an active ester chemoselective ligation group.

In certain embodiments of formula (I), Y is a reactive moiety capable forming a covalent bond to a polypeptide (e.g., with an amino acid sidechain of a polypeptide having a compatible reactive group). The reactive moiety can be referred to as a chemoselective ligation group.

In certain embodiments of formula (I), Y is a thio-reactive chemoselective ligation group (e.g., as described in Table 10). In some cases, Y can produce a residual moiety Z resulting from the covalent linkage of a thiol-reactive chemoselective ligation group to one or more cysteine residue(s) of a protein, e.g., Ab.

In certain embodiments of formula (I), Y is a Cys-reactive chemoselective ligation group (e.g., a maleimide derivative as described in table 10). In some cases, the Cys-reactive chemoselective ligation group includes a maleimide group. In some embodiments, the chemoselective ligation group includes a maleimide group of Table 10. e.g., mal-1 to mal-7.

In certain embodiments of formula (I), Y is an amino-reactive chemoselective ligation group (e.g., as described in Table 10). In some cases, Y can produce a residual moiety Z resulting from the covalent linkage of an amine-reactive chemoselective ligation group to one or more lysine residue(s) a protein, e.g., Ab.

In certain embodiments of formula (I), Y is a Lys-reactive chemoselective ligation group (e.g., an active ester as described in table 10). In some embodiments the Lys-reactive chemoselective ligation group is a PFP ester.

Exemplary chemoselective ligation groups, and synthetic precursors thereof, which may be adapted for use in the compounds of this disclosure are shown in Table 10.

TABLE 10
Exemplary chemoselective ligation groups and precursors

| Groups | Exemplary structures | |
|---|---|---|
| carboxylic acid or active ester | 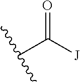 | |
| | where J is selected from —OH, —Cl, —Br, —I, —F, —OH, —O—N-succinimide, | |
| | —O-(4-nitrophenyl), —O-pentafluorophenyl, —O-tetrafluorophenyl, and | |
| | —O-—C(O)—OR^{J′}, and R^{J′} is —C₁-C₈ alkyl or -aryl, | |
| | 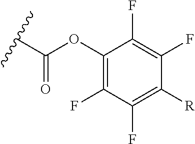 | |
| | R is H or F, | |
| | 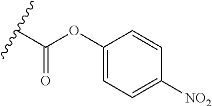 [SMILES: *C(=O)Oc1ccc([N+](=O)[O-])cc1] | 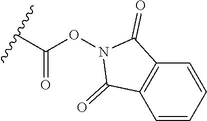 [SMILES: *C(=O)ON1C(=O)c2ccccc2C1=O] |
| | 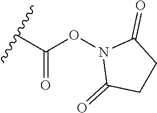 [SMILES: *C(=O)ON1C(=O)CCC1=O] | 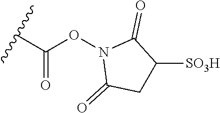 [SMILES: *C(=O)ON1C(=O)CC(S(=O)(=O)O)C1=O] |
| | 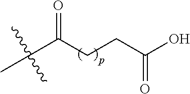 | 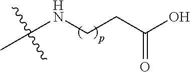 |
| | where p is 0 to 6 | |
| maleimide | 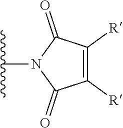 | |
| | where each R′ is independently hydrogen or halogen (e.g., bromo) | |
| | 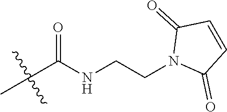 [SMILES: *C(=O)NCCN1C(=O)C=CC1=O] (mal-1) | 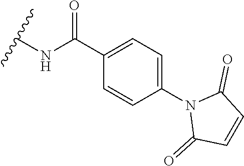 [SMILES: *NC(=O)c1ccc(N2C(=O)C=CC2=O)cc1] |
| | | (mal-2) |
| | 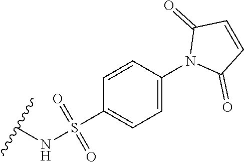 [SMILES: *NS(=O)(=O)c1ccc(N2C(=O)C=CC2=O)cc1] | 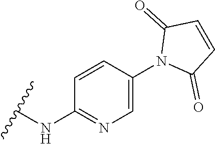 [SMILES: *Nc1ccc(N2C(=O)C=CC2=O)cn1] |
| | (mal-3) | (mal-4) |
| | 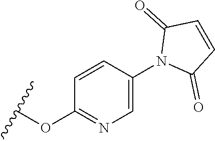 [SMILES: *Oc1ccc(N2C(=O)C=CC2=O)cn1] | 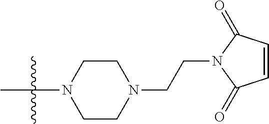 [SMILES: *N1CCN(CCN2C(=O)C=CC2=O)CC1] |
| | (mal-5) | (mal-6) |
| | 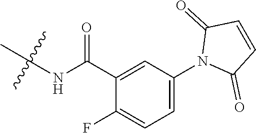 [SMILES: *NC(=O)c1cc(N2C(=O)C=CC2=O)ccc1F] | 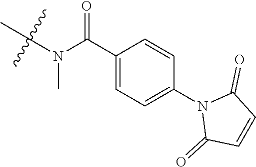 [SMILES: *N(C)C(=O)c1ccc(N2C(=O)C=CC2=O)cc1] |
| | (mal-7) | (mal-8) |
| | 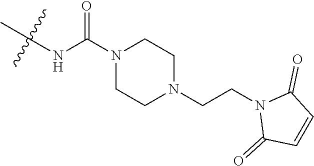 [SMILES: *NC(=O)N1CCN(CCN2C(=O)C=CC2=O)CC1] | 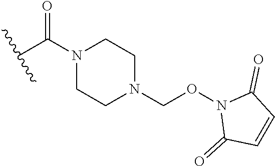 [SMILES: *C(=O)N1CCN(CON2C(=O)C=CC2=O)CC1] |
| | (mal-9) | (mal-10) |

| | 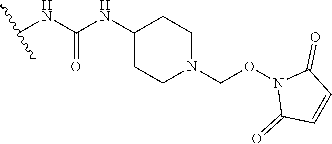 [SMILES: *NC(=O)NC1CCN(CON2C(=O)C=CC2=O)CC1] | 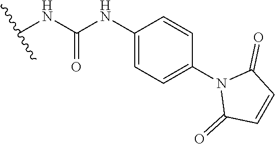 [SMILES: *NC(=O)Nc1ccc(N2C(=O)C=CC2=O)cc1] |
|---|---|---|
| | (mal-11) | (mal-12) |
| | 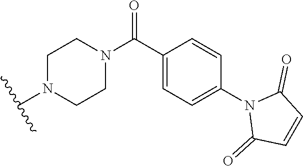 [SMILES: *N1CCN(C(=O)c2ccc(N3C(=O)C=CC3=O)cc2)CC1] (mal-13) | 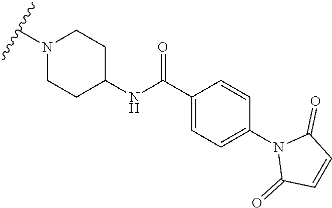 [SMILES: *N1CCC(NC(=O)c2ccc(N3C(=O)C=CC3=O)cc2)CC1] |
| | | (mal-14) |
| isocyanate or | —NCS | |
| isothiocyanate | —NCO | |
| | 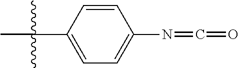 [SMILES: *c1ccc(N=C=O)cc1] | |
| | 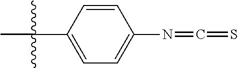 [SMILES: *c1ccc(N=C=S)cc1] | |
| alkyl halide alkyl tosylate | 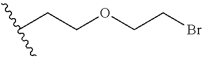 [SMILES: *CCOCCBr] | |
| | 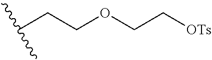 [SMILES: *CCOCCOS(=O)(=O)c1ccc(C)cc1] | |
| aldehyde | 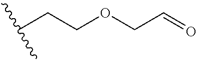 [SMILES: *CCOCC=O] | |
| haloactamide or alpha-leaving group acetamide | 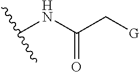 | |
| | where G is selected from —Cl, —Br, —I, —O-mesyl, and —O-tosyl | |
| haloacetamide or alpha-leaving group acetamide | 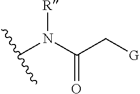 | |
| | where G is selected from —Cl, —Br, —I, —O-mesyl, and —O-tosyl; R″ is | |
| | (C₁-C₆)alkyl, heterocyclyl (e.g., 4-tetrahydro2H-pyran), or aryl | |
| | 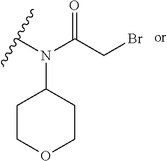 [SMILES: *N(C(=O)CBr)C1CCOCC1 ; "or"] | 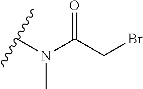 [SMILES: *N(C)C(=O)CBr] |

| 2-sulfonylpyridine | 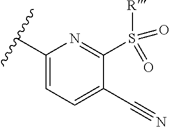 | | |
|---|---|---|---|
| | where R′′′ is alkyl | | |
| diazirine | 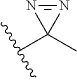 [SMILES: *C1(C)N=N1] | | |
| sulfonyl halide or vinyl sulfone | 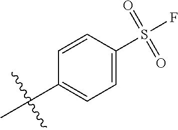 [SMILES: *c1ccc(S(=O)(=O)F)cc1] | 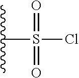 [SMILES: *S(=O)(=O)Cl] | 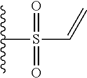 [SMILES: *S(=O)(=O)C=C] |

| | | | |
|---|---|---|---|
| hydrazide hydrazine hydroxylamino | 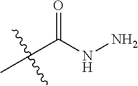 [SMILES: *C(=O)NN] | 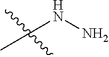 [SMILES: *NN] | 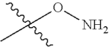 [SMILES: *ON] |

| pyridyl disulfide | 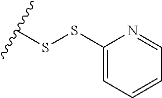 [SMILES: *SSc1ccccn1] | | |
|---|---|---|---|
| (HIPS) hydrazinyl- indolyl group, or (aza-HIPS) hydrazinyl- pyrrolo-pyridinyl group | 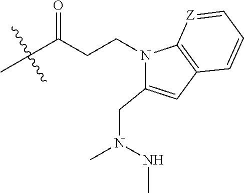 | | |
| | where Z is CH or N | | |
| alkyne or cyclooctyne | 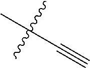 [SMILES: *C#C] | 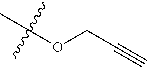 [SMILES: *OCC#C] | 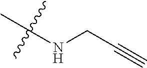 [SMILES: *NCC#C] |

| | |
|---|---|
| 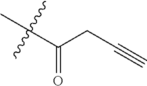 [SMILES: *C(=O)CC#C] | 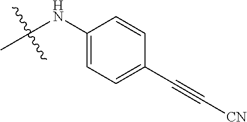 [SMILES: *Nc1ccc(C#CC#N)cc1] |
| 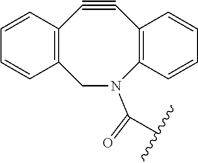 [SMILES: *C(=O)N1Cc2ccccc2C#Cc2ccccc21] | 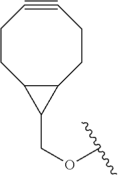 [SMILES: *OCC1C2CCC#CCCC21] |

| | | | | |
|---|---|---|---|---|
| azide | 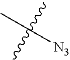 [SMILES: *N=[N+]=[N-]] | 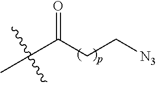 | 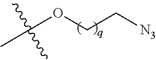 | 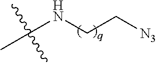 |

| | where p is 0 to 6 and where q is 1 to 6 | | | |
|---|---|---|---|---|
| amine | 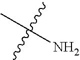 [SMILES: *N] | 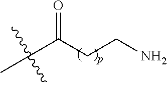 | 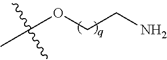 | 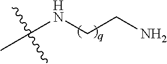 |

where p is 0 to 6 and where q is 1 to 6

In Table 10, [symbol] can represent a point of attachment of Y to a linking moiety or a linked X moiety.

[symbol]

Table 11 shows exemplary residual moieties, wherein the “***” indicates the point of attachment of Y.

TABLE 11
Exemplary residual moieties from chemoselective
ligation groups and precursors

| Groups | Exemplary residual moieties |
|---|---|
| carboxylic acid or active ester | 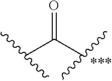 [SMILES: *C(*)=O] |
| maleimide | 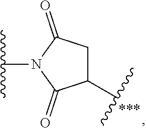 [SMILES: *C1CC(=O)N(*)C1=O] |
| | 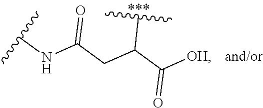 |
| | 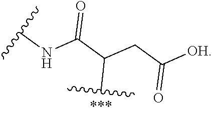 [SMILES: *NC(=O)C(*)CC(=O)O] |
| isocyanate or | Urea or thiourea |
| isothiocyanate | |
| alkyl halide | Direct bond |
| alkyl tosylate | |
| aldehyde | Imine |
| haloacetamide or | Direct bond |
| alpha-leaving group | |
| acetamide | |

Exemplary ASGPR-Binding Compounds with Chemoselective Ligation Group for Preparing Conjugates

This disclosure includes compounds of formula (I) which compounds can be prepared from a precursor ligand-linker compound including:
(1) one or more particular ASGPR ligand (X) (e.g., as described herein, such as ligands X1-X20 of Tables 1-5) or a particular ASGPR ligand (X) (e.g., as described herein),
(2) a linker including one or more linking moieties (e.g., as described herein, such as any one or more of the linking moieties of Tables 6-8); and
(3) a chemoselective ligation group (Y) e.g., as described herein, such as any one of the groups of Table 10).

Table 12 illustrates various monovalent ASGPR ligand-linker compounds for use in preparing conjugates of the disclosure.

TABLE 12
Exemplary ASGPR ligand-linker compounds for use in conjugates
Xₙ-L-Y

| | | | | effective | Chemo- |
|---|---|---|---|---|---|
| | | | | length L to | selective |
| Cmpd # | X | L | n | Y conjugate | ligation |
| 1101 (I-117) | X1 | | 1 | 10 | PFP ester |
| 1102 (I-115) | X1 | | 1 | 19 | PFP ester |
| 1103 | X1 | | 1 | 25 | PFP ester |
| 1104 (I-133) | X1 | | 1 | 29 | maleimide |
| 1105 | X1 | | 1 | 28 | PFP ester |
| 1106 (I-112) | X1 | | 1 | 26 | PFP ester |
| 1107 (I-146) | X1 | | 1 | 35 | PFP ester |
| 1108 (I-118) | X6 | | 1 | 10 | PFP ester |
| 1109 (I-116) | X6 | | 1 | 19 | PFP ester |
| 1110 (I-113) | X6 | | 1 | 26 | NHS ester |
| 1111 (I-147 ) | X5 | L10 | 1 | 27 | PFP ester |
| 1112 (I-148) | X5 | L1 | 1 | 26 | PFP ester |
| 1113 (I-149) | X1 | L11 | 1 | 23 | PFP ester |
| 1114 (I-150) | X1 | L11 | 1 | 26 | PFP ester |
| 1115 (I-151) | X1 | L11 | 1 | 29 | PFP ester |

PFP is pentafluorophenyl
TFP is tetrafluorophenyl
NHS is N-hydroxysuccinimde ester

Tables 13 illustrates various multivalent ASGPR ligand-linker compounds for use in conjugates of the disclosure.

TABLE 13
Exemplary Multimeric ASGPR Ligand-linker compounds
Xₙ-L-Y

| | | | X¹ to | branch to | |
|---|---|---|---|---|---|
| Cmpd # | X | n | branch length | Y length | Y |
| 1218 (I-143) | X4 | 3 | 14 to C | 12 to C═O | PFP ester |
| 1303 (I-136) | X8 | 3 | 16 to C | 12 to C═O | PFP ester |
| 1219 (I-157) | X4 | 3 | 6 to N | 12 to C═O | PFP ester |
| 1213 (I-137) | X1 | 3 | 15 to C | 81 to C═O | PFP ester |
| 1211 (I-129) | X1 | 3 | 15 to C | 33 to C═O | PFP ester |
| 1203 (I-144) | X1 | 2 | 15 to CH | 12 to C═O | PFP ester |
| 1215 (I-141) | X2 | 3 | 15 to C | 12 to C═O | PFP ester |
| 1208 (I-145) | X1 | 3 | 15 to C | 16 to C═O | PFP ester |
| 1216 (I-140) | X3 | 3 | 18 to C | 12 to C-O | PFP ester |
| 1401 (I-153) | X10 | 3 | 14 to C | 12 to C═O | PFP ester |
| 1204 (I-111) | X1 | 2 | 16 to N | 11 to C═O | PFP ester |
| 1402 (I-154) | X11 | 3 | 19 to C | 12 to C═O | PFP ester |
| 1403 (I-155) | X12 | 3 | 19 to C | 12 to C═O | PFP ester |
| 1246 | X2 | 3 | 17 to C | 12 to phenyl group | mal-2 |
| | (α) | | | | |
| 1248 | X3 | 3 | 15 to C | 17 to piperazine | mal-6 |
| | (α) | | | | |
| 1250 | X3 | 3 | 17 to C | 19 to C═O | mal-1 |
| | (α) | | | | |
| 1225A | X2 | 3 | 16 to C | 12 to C═O | mal-1 |
| | (a) | | | | |
| 1251 | X3 | 3 | 17 to C | 12 to C═O | mal-1 |
| | (α) | | | | |
| 1252 | X1 | 3 | 18 to C | 18 to phenyl | mal-2 |
| 1249 | X3 | 3 | 17 to C | 17 to piperazine | mal-6 |
| | (α) | | | | |
| 1258 | X3 | 3 | 21 to C | 18 to phenyl | mal-2 |
| | (a) | | | | |
| 1921 | X22 | 3 | 18 to C | 18 to phenyl | mal-2 |
| 1253 | X2 | 3 | 15 to C | 18 to phenyl | mal-2 |
| | (α) | | | | |
| 1255 | X3 | 3 | 16 to C | 18 to phenyl | mal-2 |
| | (β) | | | | |
| 1257 | X2 | 3 | 17 to C | 19 to phenyl | mal-7 |
| | (α) | | | | |
| 1247 | X3 | 3 | 18 to C | 18 to phenyl | mal-2 |
| | (α) | | | | |
| 1254 | X2 | 3 | 17 to C | 18 to phenyl | mal-2 |
| | (α) | | | | |
| 1259 | X3 | 3 | 16 to C | 18 to phenyl | mal-2 |
| | (α) | | | | |
| 1915A | X25 | 3 | 19 to C | 18 to phenyl | mal-2 |

Tables 14-17 illustrate several exemplary ASGPR binding compounds of this disclosure that include a chemoselective ligation group, or a precursor thereof. It is understood that this disclosure includes Y (e.g., as described herein) conjugates of each of the exemplary compounds of Tables 14-17. For example, conjugates where the chemoselective ligation group has been conjugated to a different Y, such as an antibody or antibody fragment for a target protein.

The chemoselective ligation group of such compounds can be utilized to connect to another Y moiety of interest (e.g., as described below). It is understood that any of these compounds can also be prepared de novo to include an alternative Y moiety of interest (e.g., as described below) rather than the chemoselective ligation group. In some embodiments, such compounds are referred to as a conjugate, e.g., a biomolecule conjugate that specifically binds a target protein.

TABLE 14
Example ASGPR binding compounds having chemoselective ligation group

| # | Structure |
|---|---|
| 1101 (I-117) | 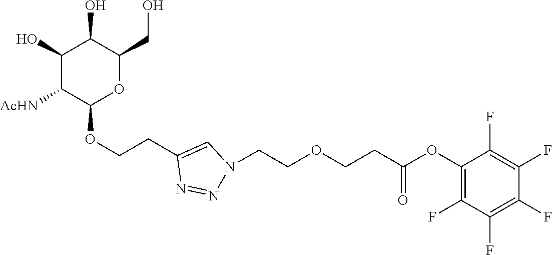 [SMILES: CC(=O)N[C@H]1[C@H](OCCc2cn(CCOCCC(=O)Oc3c(F)c(F)c(F)c(F)c3F)nn2)O[C@H](CO)[C@H](O)[C@@H]1O] |
| 1102 (I-115) | 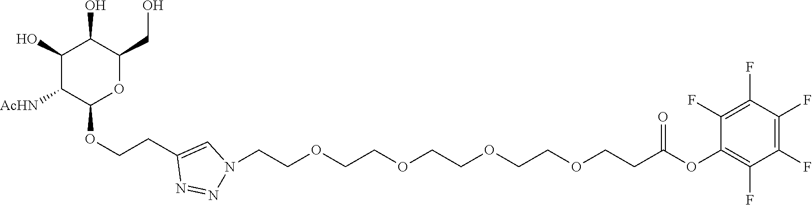 [SMILES: CC(=O)N[C@H]1[C@H](OCCc2cn(CCOCCOCCOCCOCCC(=O)Oc3c(F)c(F)c(F)c(F)c3F)nn2)O[C@H](CO)[C@H](O)[C@@H]1O] |
| 1103 | 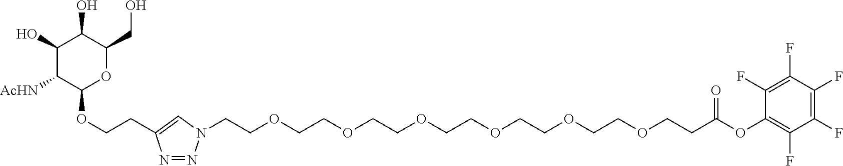 [SMILES: CC(=O)N[C@H]1[C@H](OCCc2cn(CCOCCOCCOCCOCCOCCOCCC(=O)Oc3c(F)c(F)c(F)c(F)c3F)nn2)O[C@H](CO)[C@H](O)[C@@H]1O] |
| 1104 (I-133) | 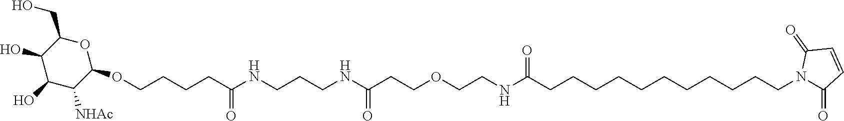 [SMILES: CC(=O)N[C@H]1[C@H](OCCCCC(=O)NCCCNC(=O)CCOCCNC(=O)CCCCCCCCCCCN2C(=O)C=CC2=O)O[C@H](CO)[C@H](O)[C@@H]1O] |
| 1105 | 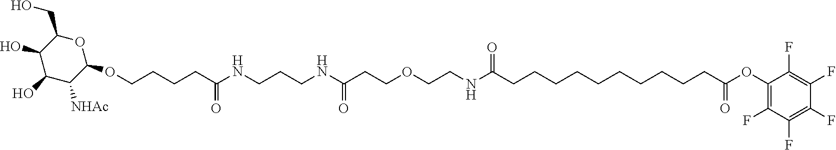 [SMILES: CC(=O)N[C@H]1[C@H](OCCCCC(=O)NCCCNC(=O)CCOCCNC(=O)CCCCCCCCCCC(=O)Oc2c(F)c(F)c(F)c(F)c2F)O[C@H](CO)[C@H](O)[C@@H]1O] |
| 1106 (I-112) | 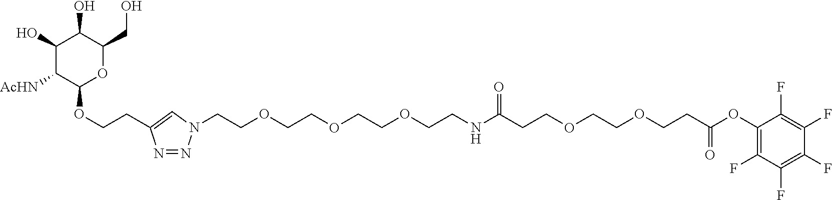 [SMILES: CC(=O)N[C@H]1[C@H](OCCc2cn(CCOCCOCCOCCNC(=O)CCOCCOCCC(=O)Oc3c(F)c(F)c(F)c(F)c3F)nn2)O[C@H](CO)[C@H](O)[C@@H]1O] |
| 1107 (I-146) | 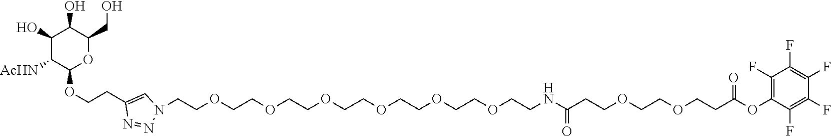 [SMILES: CC(=O)N[C@H]1[C@H](OCCc2cn(CCOCCOCCOCCOCCOCCOCCNC(=O)CCOCCOCCC(=O)Oc3c(F)c(F)c(F)c(F)c3F)nn2)O[C@H](CO)[C@H](O)[C@@H]1O] |
| 1108 (I-118) | 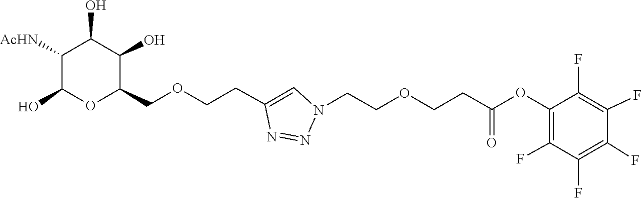 [SMILES: CC(=O)N[C@@H]1[C@@H](O)[C@@H](O)[C@@H](COCCc2cn(CCOCCC(=O)Oc3c(F)c(F)c(F)c(F)c3F)nn2)O[C@H]1O] |
| 1109 (I-116) | 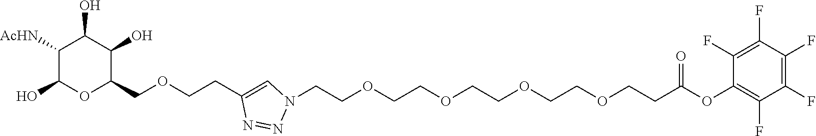 [SMILES: CC(=O)N[C@@H]1[C@@H](O)[C@@H](O)[C@@H](COCCc2cn(CCOCCOCCOCCOCCC(=O)Oc3c(F)c(F)c(F)c(F)c3F)nn2)O[C@H]1O] |
| 1110 (I-113) | 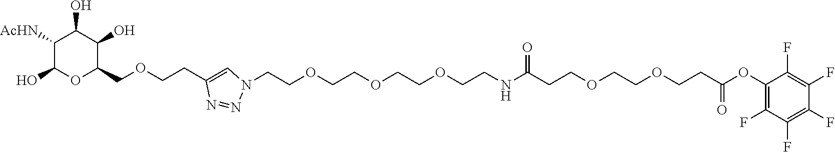 [SMILES: CC(=O)N[C@@H]1[C@@H](O)[C@@H](O)[C@@H](COCCc2cn(CCOCCOCCOCCNC(=O)CCOCCOCCC(=O)Oc3c(F)c(F)c(F)c(F)c3F)nn2)O[C@H]1O] |
| 1111 (I-147) | 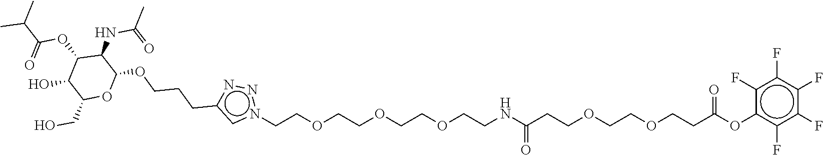 |
| 1112 (I-148) | 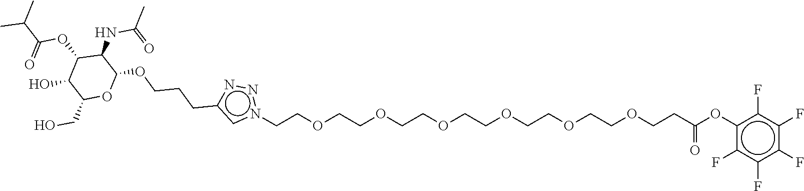 |
| 1113 (I-149) | 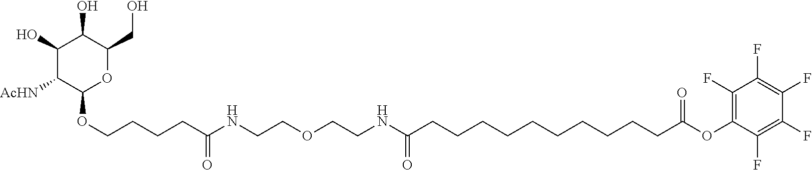 [SMILES: CC(=O)N[C@H]1[C@H](OCCCCC(=O)NCCOCCNC(=O)CCCCCCCCCCC(=O)Oc2c(F)c(F)c(F)c(F)c2F)O[C@H](CO)[C@H](O)[C@@H]1O] |
| 1114 (I-150) | 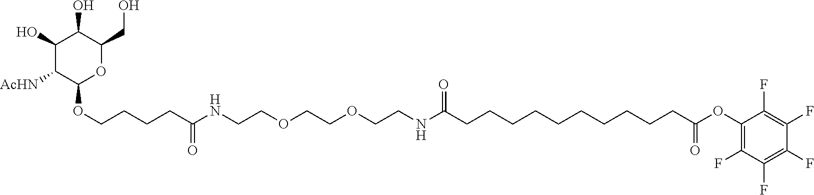 [SMILES: CC(=O)N[C@H]1[C@H](OCCCCC(=O)NCCOCCOCCNC(=O)CCCCCCCCCCC(=O)Oc2c(F)c(F)c(F)c(F)c2F)O[C@H](CO)[C@H](O)[C@@H]1O] |
| 1115 (I-151) | 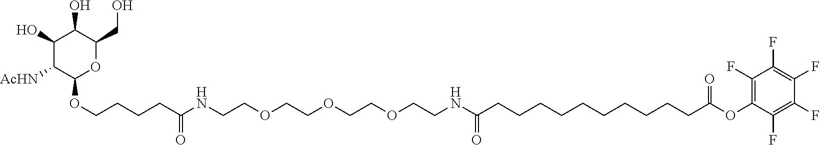 [SMILES: CC(=O)N[C@H]1[C@H](OCCCCC(=O)NCCOCCOCCOCCNC(=O)CCCCCCCCCCC(=O)Oc2c(F)c(F)c(F)c(F)c2F)O[C@H](CO)[C@H](O)[C@@H]1O] |
| 1116 (I-164) | 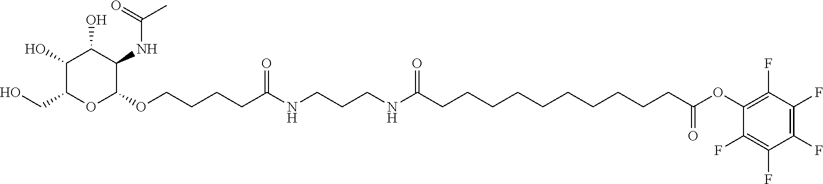 [SMILES: CC(=O)N[C@H]1[C@H](OCCCCC(=O)NCCCNC(=O)CCCCCCCCCCC(=O)Oc2c(F)c(F)c(F)c(F)c2F)O[C@H](CO)[C@H](O)[C@@H]1O] |
| 1117 (I-168) | 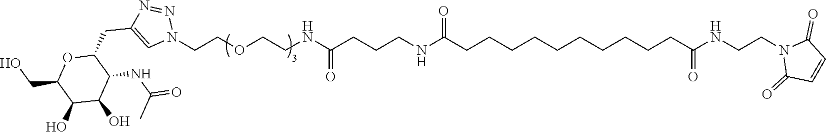 |
| 1118 (I-169) | 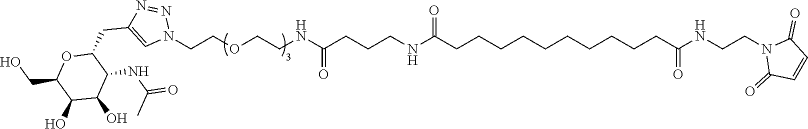 |
| 1119 | 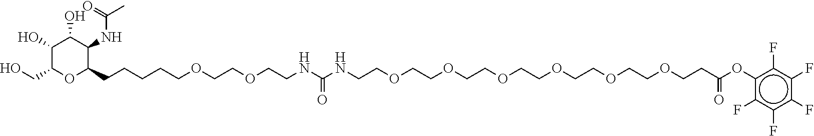 |
| 1120 | 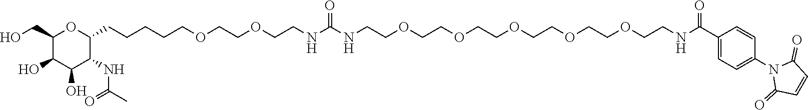 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)c1ccc(N2C(=O)C=CC2=O)cc1] |
| 1121 | 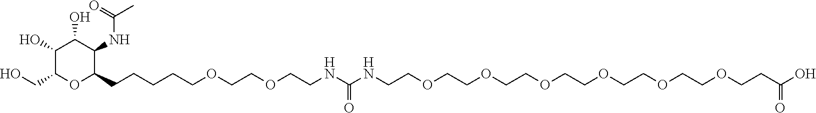 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)O] |
| 1122 | 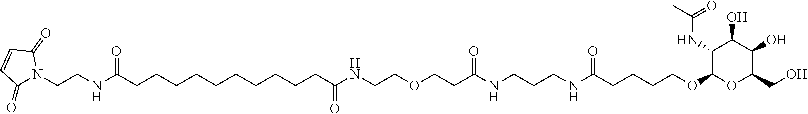 [SMILES: CC(=O)N[C@H]1[C@H](OCCCCC(=O)NCCCNC(=O)CCOCCNC(=O)CCCCCCCCCCC(=O)NCCN2C(=O)C=CC2=O)O[C@H](CO)[C@H](O)[C@@H]1O] |
| 1123 | 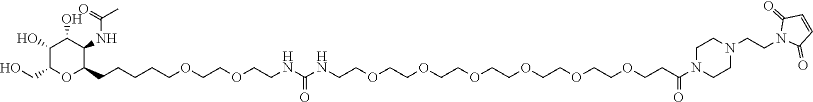 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)N1CCN(CCN2C(=O)C=CC2=O)CC1] |
| 1124 | 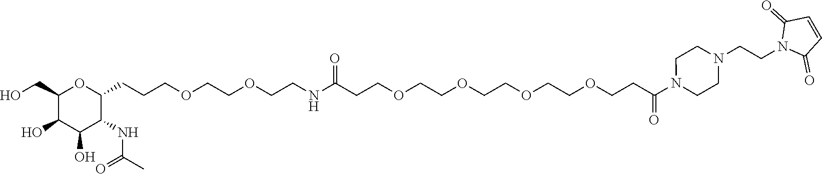 [SMILES: CC(=O)N[C@@H]1[C@@H](O)[C@@H](O)[C@@H](CO)O[C@@H]1CCCOCCOCCNC(=O)CCOCCOCCOCCOCCC(=O)N1CCN(CCN2C(=O)C=CC2=O)CC1] |

TABLE 15
Multivalent ASGPR binding compounds having chemoselective ligation group and X
group of formula (Ib)

| # | Structure |
|---|---|
| 1201 | 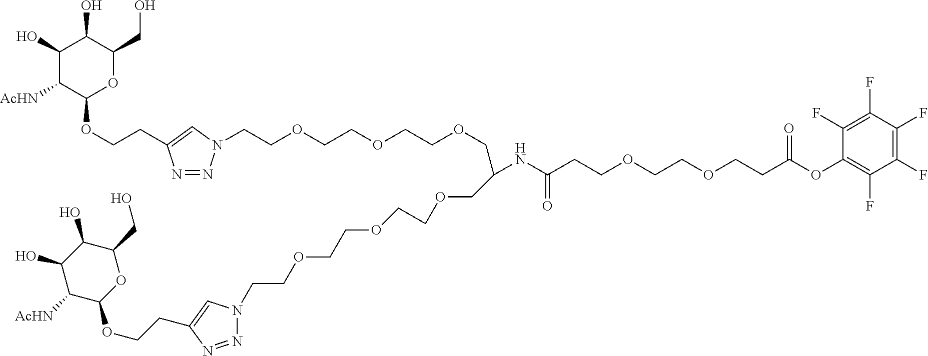 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O] |
| 1202 (I-131) | 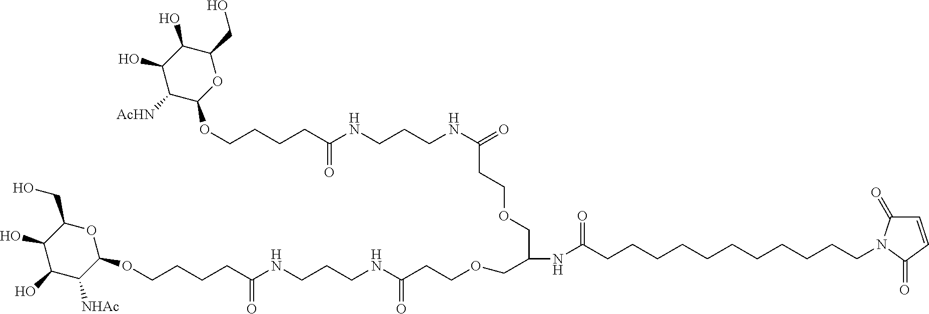 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 1203 (I-144) | 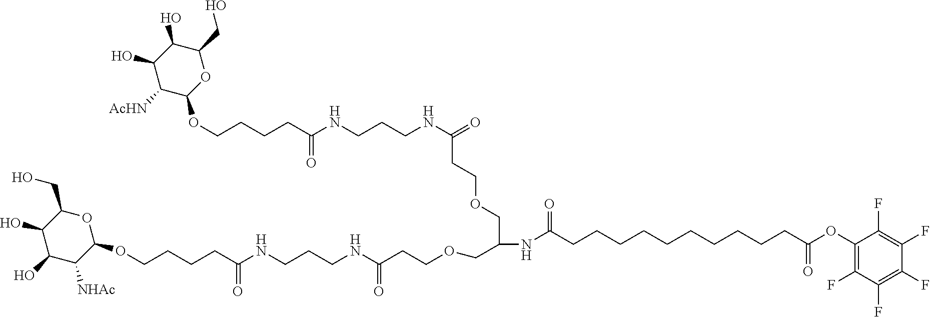 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 1204 (I-111) | 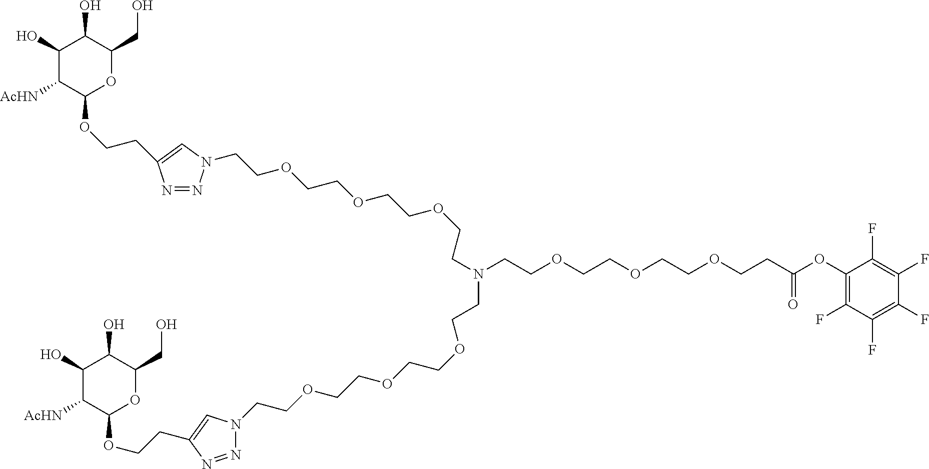 [SMILES: CC(=O)N[C@H]1[C@H](OCCc2cn(CCOCCOCCOCCN(CCOCCOCCOCCC(=O)Oc3c(F)c(F)c(F)c(F)c3F)CCOCCOCCOCCn3cc(CCO[C@@H]4O[C@H](CO)[C@H](O)[C@H](O)[C@H]4NC(C)=O)nn3)nn2)O[C@H](CO)[C@H](O)[C@@H]1O] |
| 1217 | 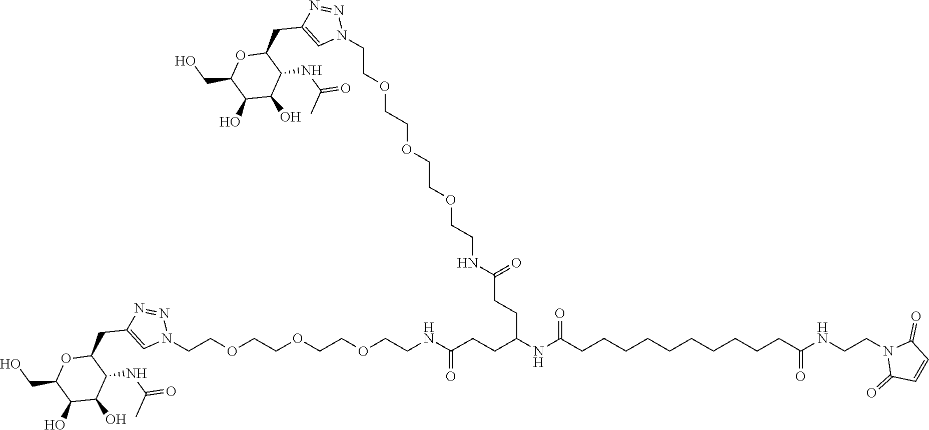 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1217A | 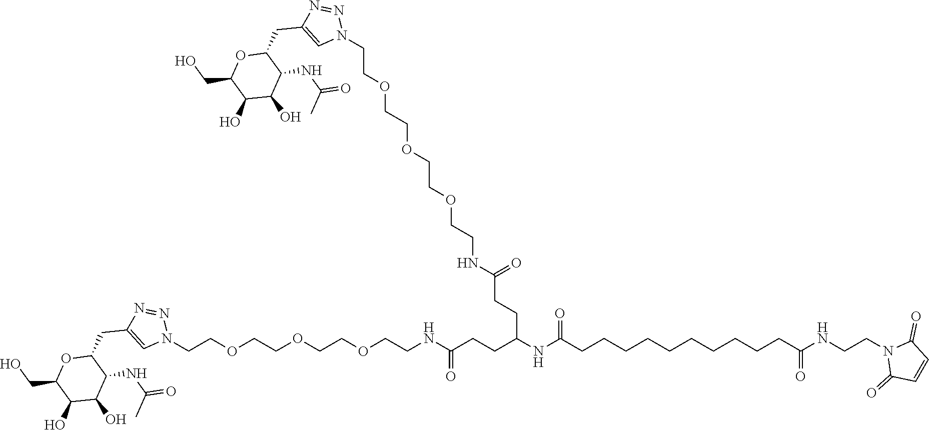 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1235 | 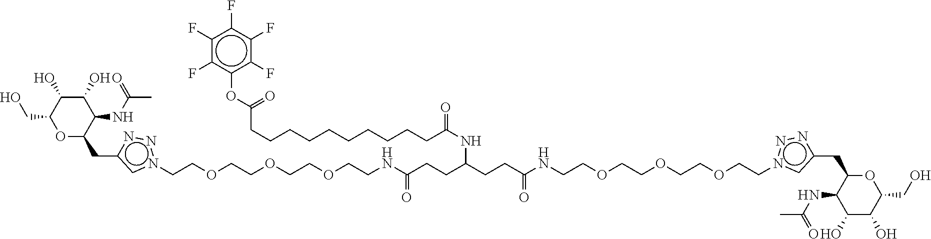 |
| 1236 | 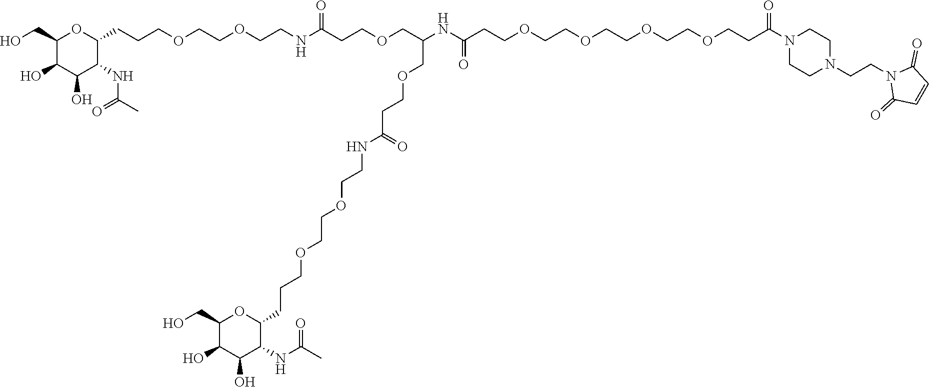 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)NC(=O)CCOCCOCCOCCOCCC(=O)N1CCN(CCN2C(=O)C=CC2=O)CC1] |
| 1236A | 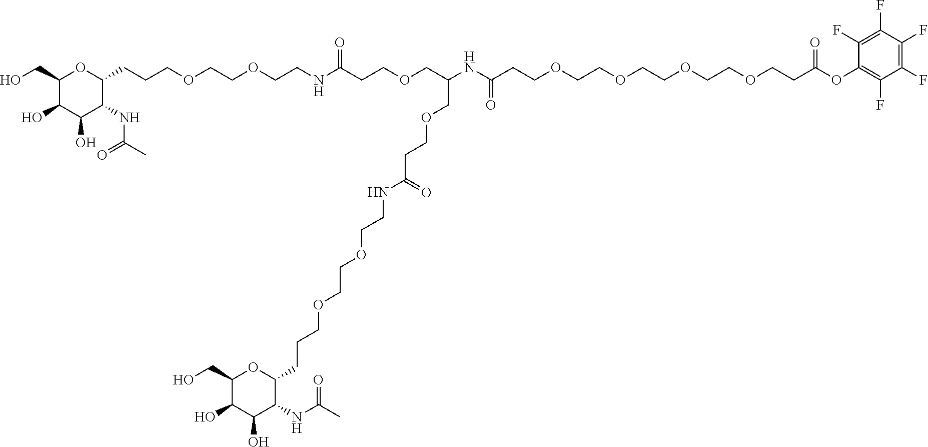 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 1237 | 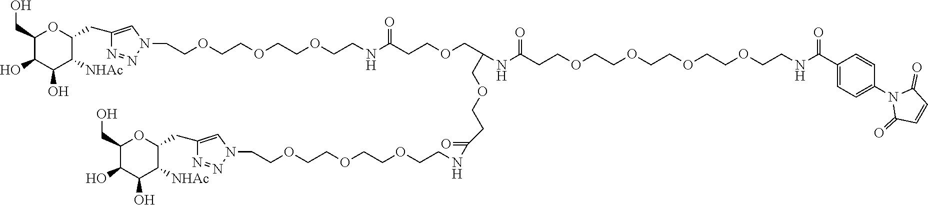 [SMILES: CC(=O)N[C@@H]1[C@@H](O)[C@@H](O)[C@@H](CO)O[C@@H]1Cc1cn(CCOCCOCCOCCNC(=O)CCOCC(COCCC(=O)NCCOCCOCCOCCn2cc(C[C@H]3O[C@H](CO)[C@H](O)[C@H](O)[C@H]3NC(C)=O)nn2)NC(=O)CCOCCOCCOCCOCCNC(=O)c2ccc(N3C(=O)C=CC3=O)cc2)nn1] |
| 1238 | 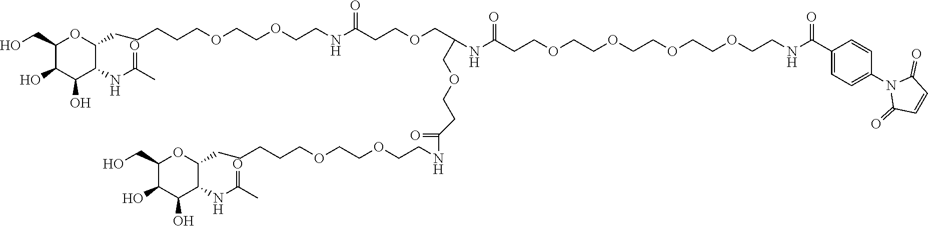 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1239 | 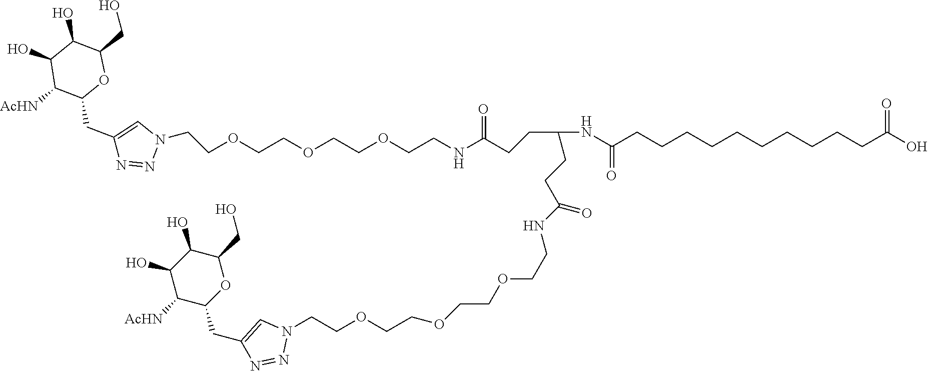 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)O)nn1] |
| 1240 | 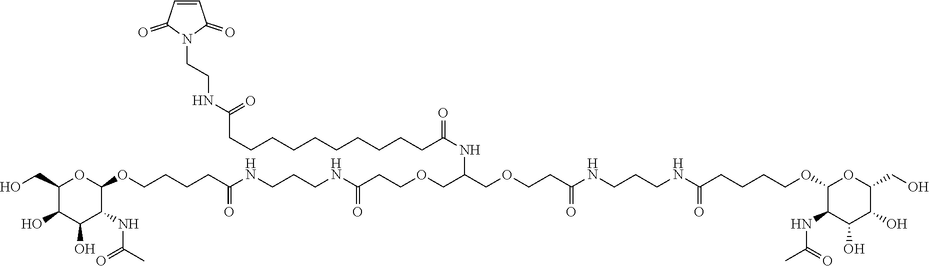 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O] |
| 1241 | 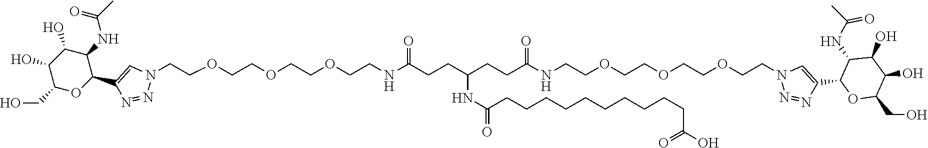 [SMILES: CC(=O)N[C@@H]1[C@@H](O)[C@@H](O)[C@@H](CO)O[C@@H]1c1cn(CCOCCOCCOCCNC(=O)CCC(CCC(=O)NCCOCCOCCOCCn2cc([C@H]3O[C@H](CO)[C@H](O)[C@H](O)[C@H]3NC(C)=O)nn2)NC(=O)CCCCCCCCCCC(=O)O)nn1] |
| 1242 | 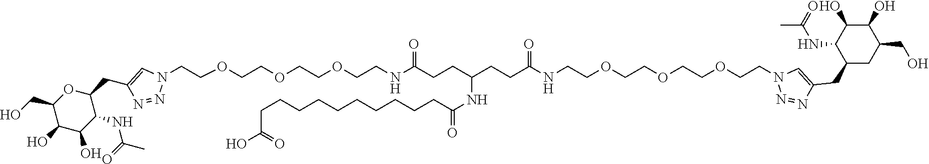 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3C[C@H](CO)[C@H](O)[C@H](O)[C@H]3NC(C)=O)nn2)NC(=O)CCCCCCCCCCC(=O)O)nn1] |

Trimeric ligands

| | |
|---|---|
| 1205 (I-127) | 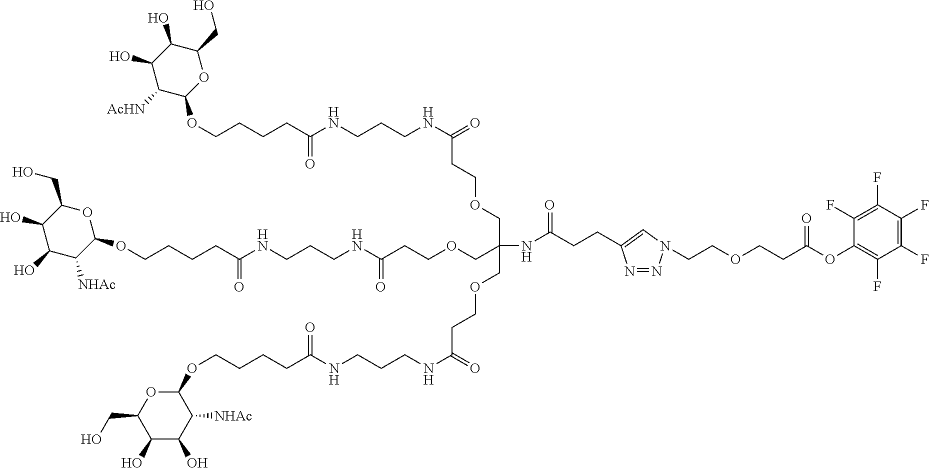 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCc2cn(CCOCCC(=O)Oc3c(F)c(F)c(F)c(F)c3F)nn2)O[C@H](CO)[C@H](O)[C@@H]1O] |
| 1206 | 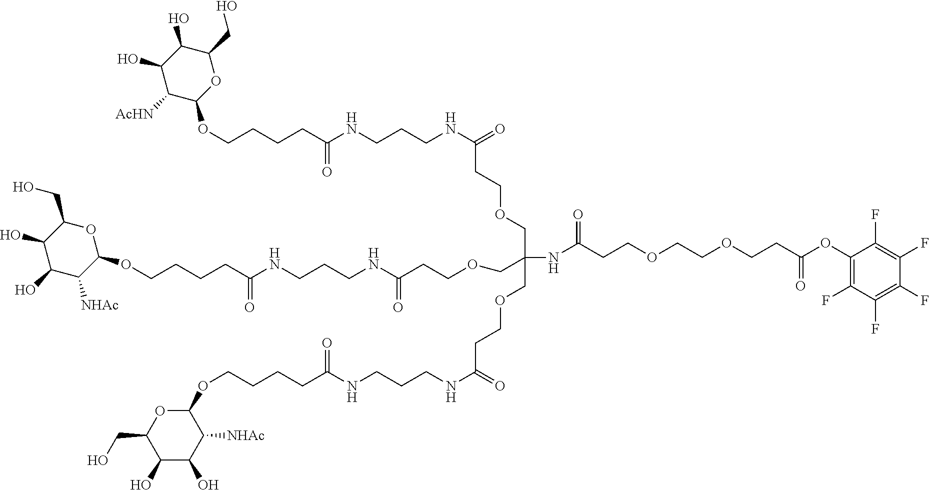 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCC(=O)Oc2c(F)c(F)c(F)c(F)c2F)O[C@H](CO)[C@H](O)[C@@H]1O] |
| 1207 (I-107) | 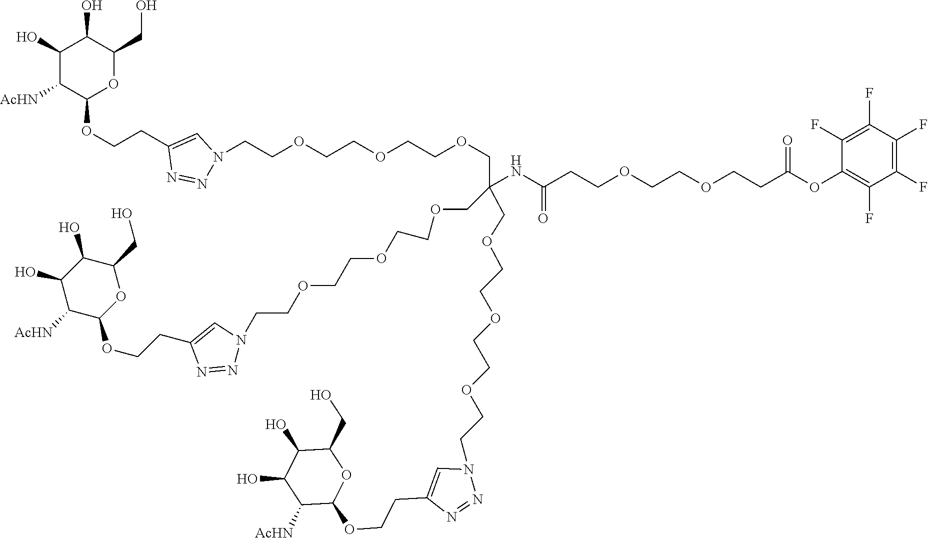 [SMILES: CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O] |
| 1208 (I-145) | 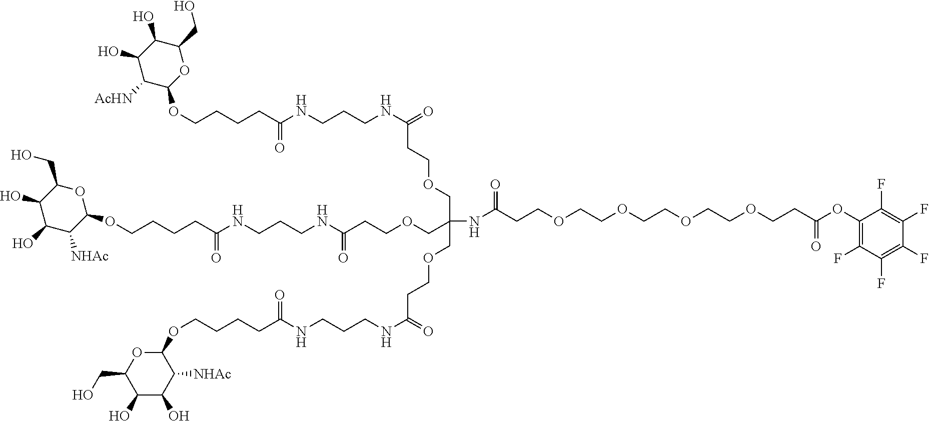 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCC(=O)Oc2c(F)c(F)c(F)c(F)c2F)O[C@H](CO)[C@H](O)[C@@H]1O] |
| 1209 | |
| (I-124) | |
| 1209-C | 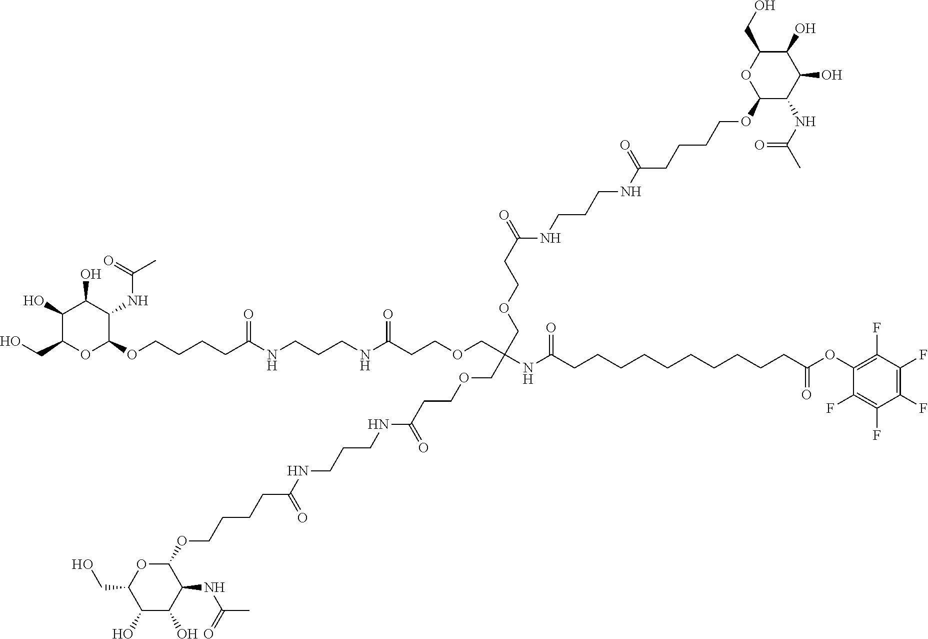 [SMILES: CC(=O)N[C@@H]1[C@@H](OCCCCC(=O)NCCCNC(=O)CCOCC(COCCC(=O)NCCCNC(=O)CCCCO[C@H]2O[C@@H](CO)[C@@H](O)[C@@H](O)[C@@H]2NC(C)=O)(COCCC(=O)NCCCNC(=O)CCCCO[C@H]2O[C@@H](CO)[C@@H](O)[C@@H](O)[C@@H]2NC(C)=O)NC(=O)CCCCCCCCCCC(=O)Oc2c(F)c(F)c(F)c(F)c2F)O[C@@H](CO)[C@@H](O)[C@H]1O] |
| 1210 | |
| (I-123) | |
| 1210A | 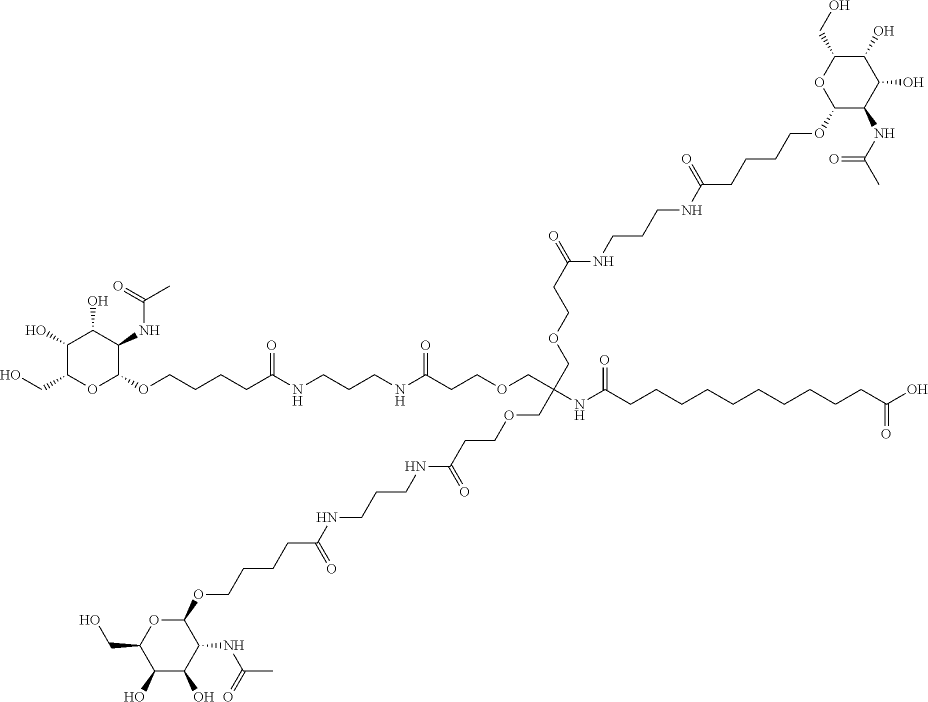 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)O)O[C@H](CO)[C@H](O)[C@@H]1O] |
| 1211 (I-129) | 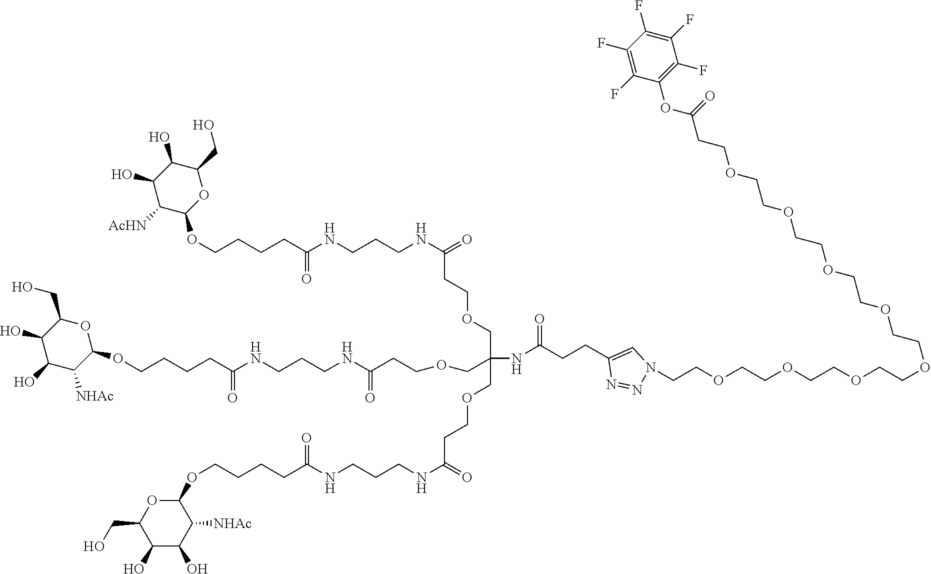 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCc2cn(CCOCCOCCOCCOCCOCCOCCOCCOCCC(=O)Oc3c(F)c(F)c(F)c(F)c3F)nn2)O[C@H](CO)[C@H](O)[C@@H]1O] |
| 1212 I-125 | 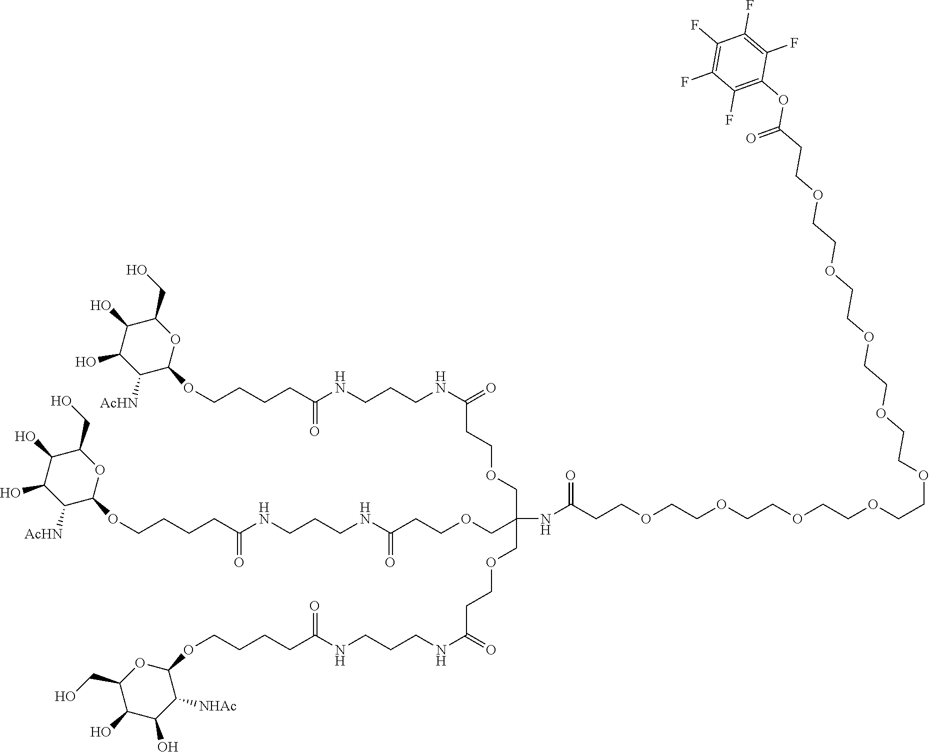 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCOCCOCCOCCOCCOCCC(=O)Oc2c(F)c(F)c(F)c(F)c2F)O[C@H](CO)[C@H](O)[C@@H]1O] |
| 1213 (I-137) | 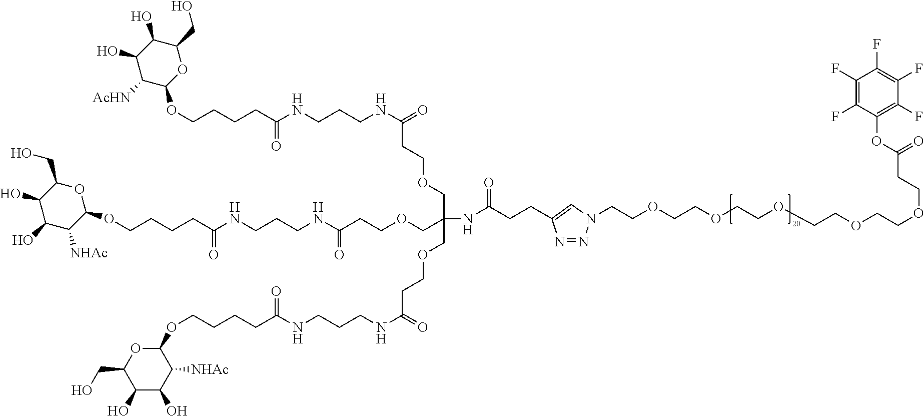 |
| 1214 (I-135) | 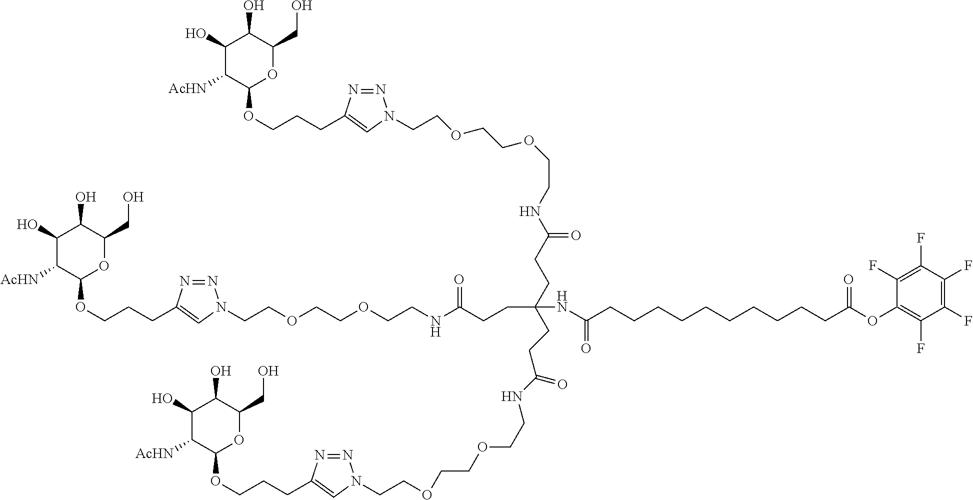 [SMILES: CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](O)[C@H]4NC(C)=O)nn3)(CCC(=O)NCCOCCOCCn3cc(CCCO[C@@H]4O[C@H](CO)[C@H](O)[C@H](O)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1O] |
| 1215 (I-141) | 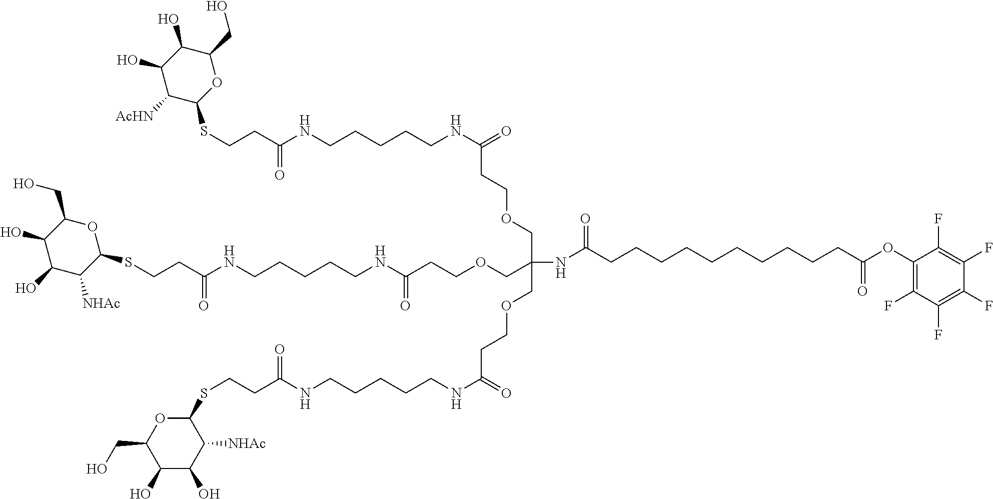 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCCCCCCCCCC(=O)Oc1c(F)c(F)c(F)c(F)c1F] |
| 1216 (I-140) | 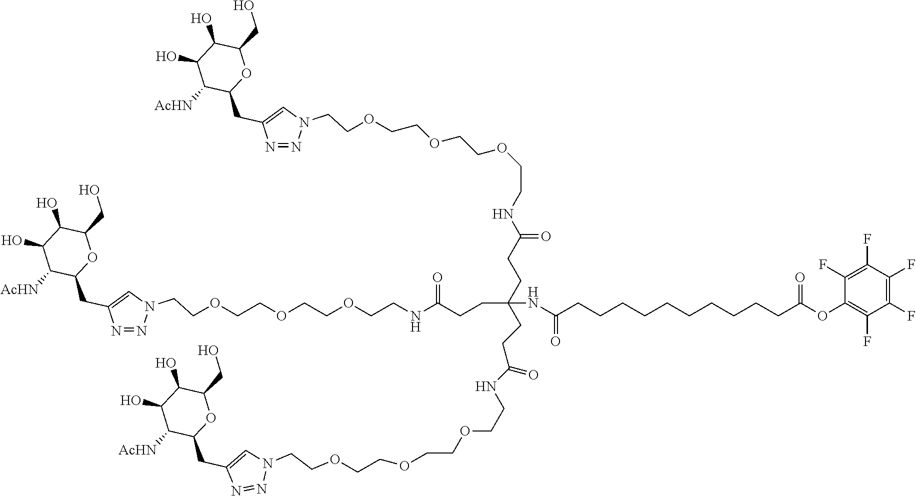 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1216A | 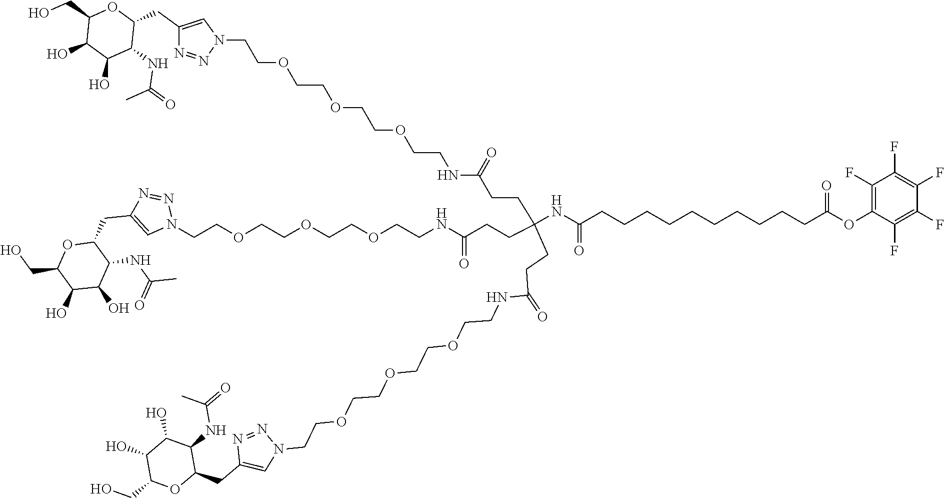 [SMILES: CC(=O)N[C@@H]1[C@@H](O)[C@@H](O)[C@@H](CO)O[C@@H]1Cc1cn(CCOCCOCCOCCNC(=O)CCC(CCC(=O)NCCOCCOCCOCCn2cc(C[C@H]3O[C@H](CO)[C@H](O)[C@H](O)[C@H]3NC(C)=O)nn2)(CCC(=O)NCCOCCOCCOCCn2cc(C[C@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1218 (I-143) | 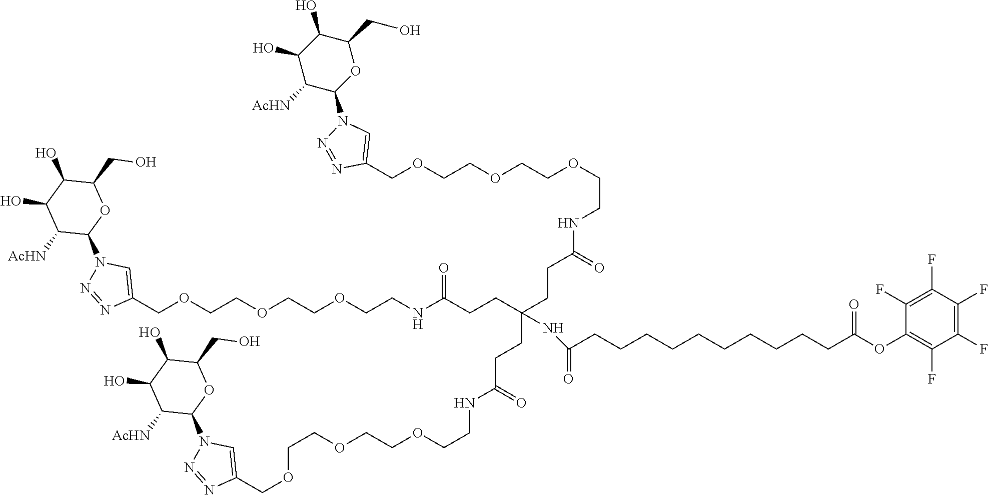 [SMILES: CC(=O)N[C@@H]1[C@@H](O)[C@@H](O)[C@@H](CO)O[C@H]1n1cc(COCCOCCOCCNC(=O)CCC(CCC(=O)NCCOCCOCCOCc2cn([C@@H]3O[C@H](CO)[C@H](O)[C@H](O)[C@H]3NC(C)=O)nn2)(CCC(=O)NCCOCCOCCOCc2cn([C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1219 (I-157) | 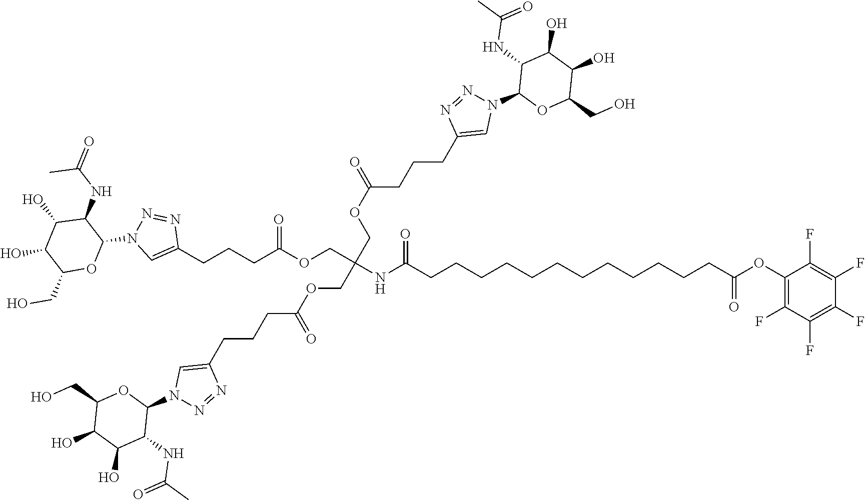 [SMILES: CC(=O)N[C@@H]1[C@@H](O)[C@@H](O)[C@@H](CO)O[C@H]1n1cc(CCCC(=O)OCC(COC(=O)CCCc2cn([C@@H]3O[C@H](CO)[C@H](O)[C@H](O)[C@H]3NC(C)=O)nn2)(COC(=O)CCCc2cn([C@@H]3O[C@H](CO)[C@H](O)[C@H](O)[C@H]3NC(C)=O)nn2)NC(=O)CCCCCCCCCCCCC(=O)Oc2c(F)c(F)c(F)c(F)c2F)nn1] |
| 1220 (I-158) | 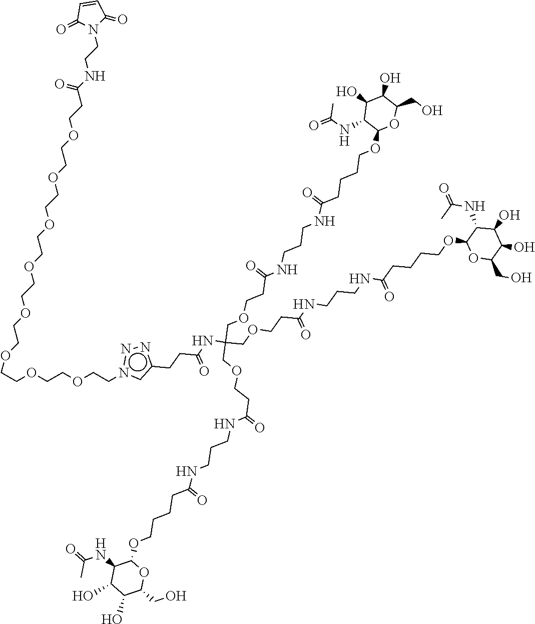 |
| 1221 (I-138) | 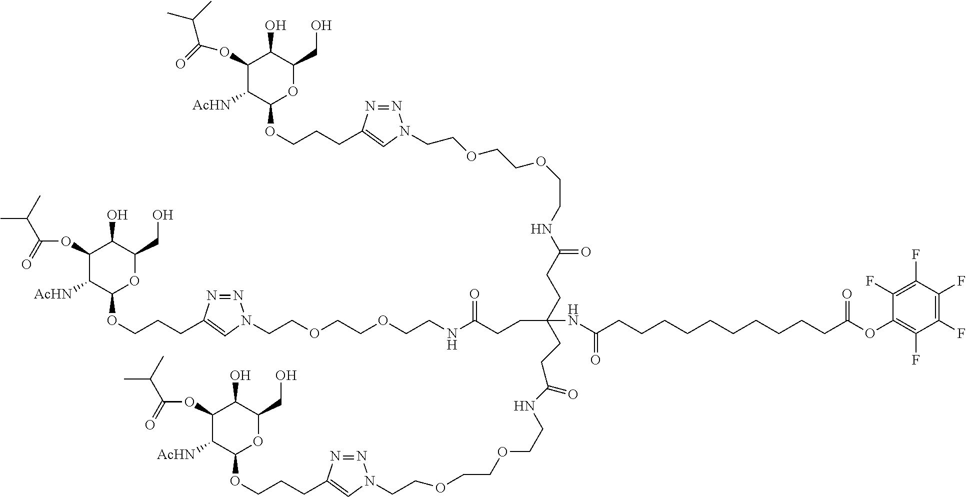 [SMILES: CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](OC(=O)C(C)C)[C@H]4NC(C)=O)nn3)(CCC(=O)NCCOCCOCCn3cc(CCCO[C@@H]4O[C@H](CO)[C@H](O)[C@H](OC(=O)C(C)C)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1OC(=O)C(C)C] |
| 1222 (I-159) | 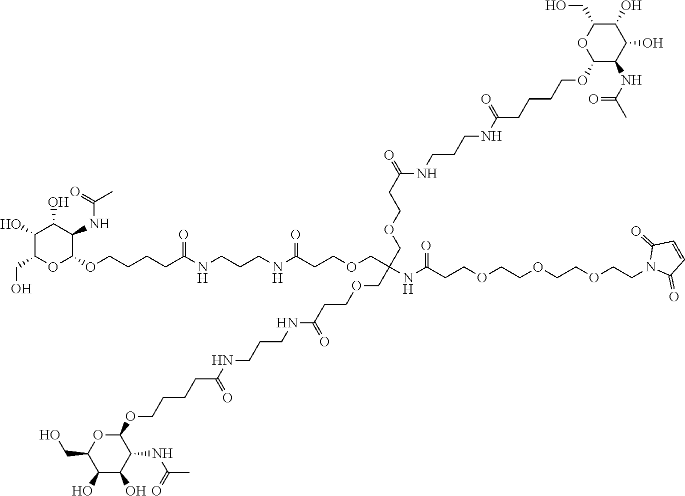 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCN2C(=O)C=CC2=O)O[C@H](CO)[C@H](O)[C@@H]1O] |
| 1223 (I-160) | 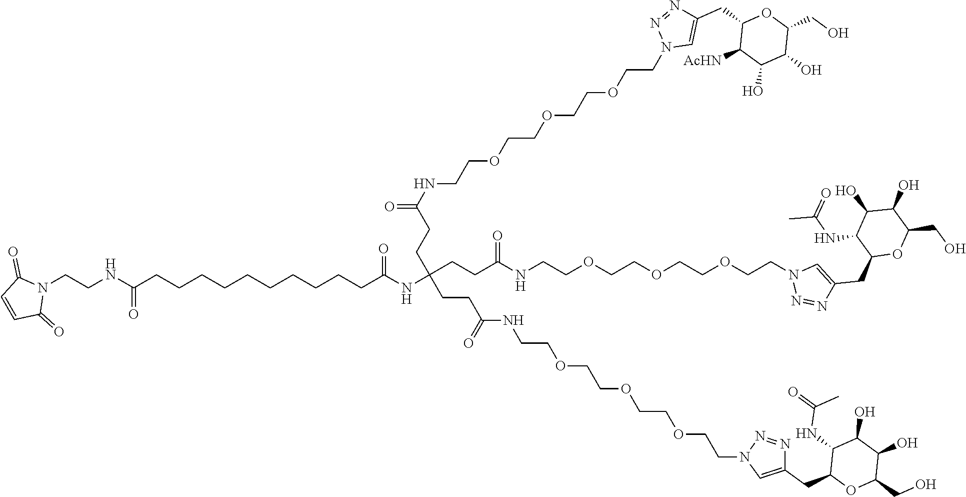 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1224 (I-161) | 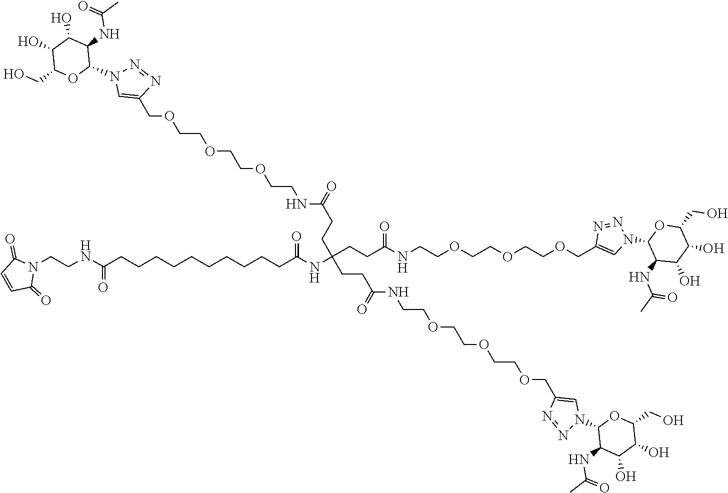 [SMILES: CC(=O)N[C@@H]1[C@@H](O)[C@@H](O)[C@@H](CO)O[C@H]1n1cc(COCCOCCOCCNC(=O)CCC(CCC(=O)NCCOCCOCCOCc2cn([C@@H]3O[C@H](CO)[C@H](O)[C@H](O)[C@H]3NC(C)=O)nn2)(CCC(=O)NCCOCCOCCOCc2cn([C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1225 (I-162) | 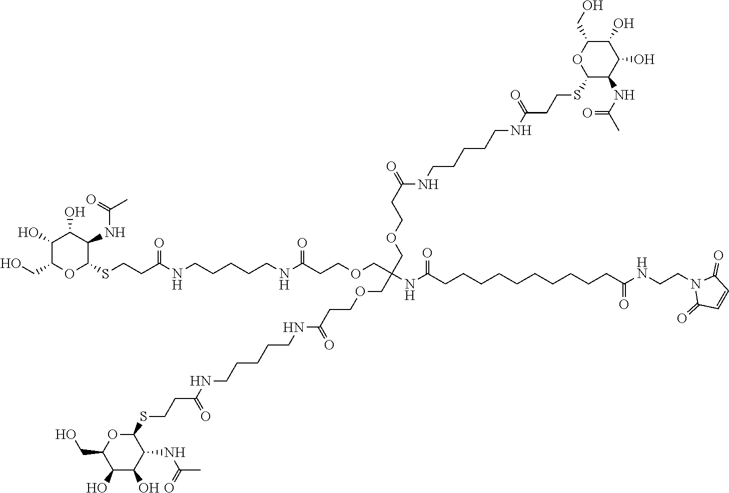 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCCCCCCCCCC(=O)NCCN1C(=O)C=CC1=O] |
| 1225A | 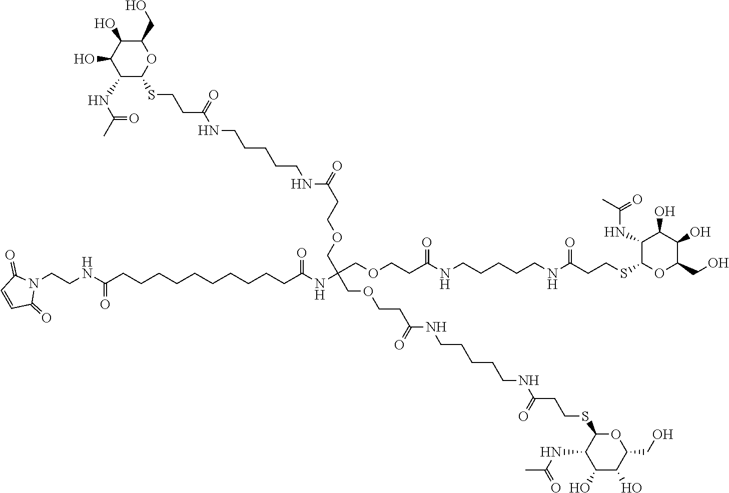 [SMILES: CC(=O)N[C@@H]1[C@@H](O)[C@@H](O)[C@@H](CO)O[C@@H]1SCCC(=O)NCCCCCNC(=O)CCOCC(COCCC(=O)NCCCCCNC(=O)CCS[C@H]1O[C@H](CO)[C@H](O)[C@H](O)[C@H]1NC(C)=O)(COCCC(=O)NCCCCCNC(=O)CCS[C@H]1O[C@H](CO)[C@H](O)[C@H](O)[C@@H]1NC(C)=O)NC(=O)CCCCCCCCCCC(=O)NCCN1C(=O)C=CC1=O] |
| 1226 (I-163) | 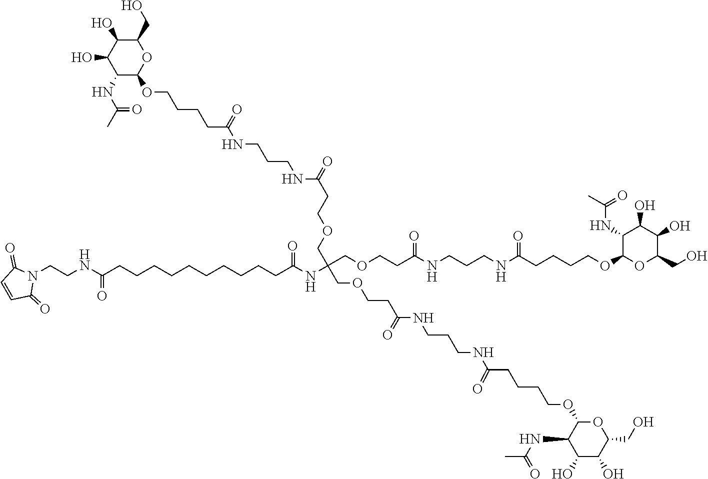 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O] |
| Com- pound Y | 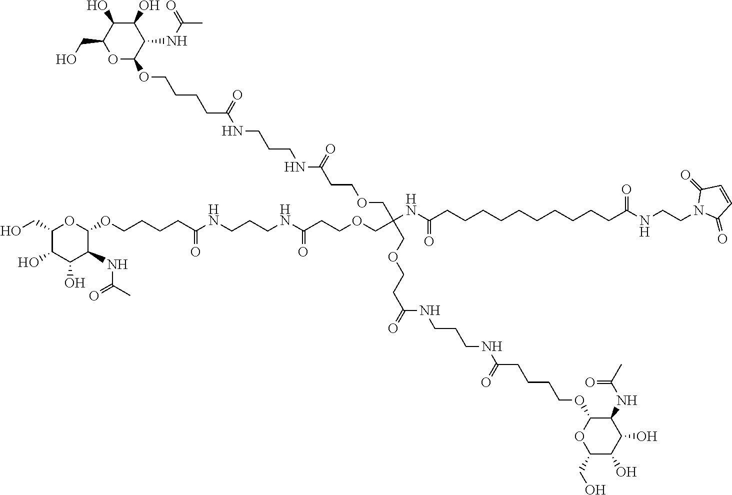 [SMILES: CC(=O)N[C@@H]1[C@@H](OCCCCC(=O)NCCCNC(=O)CCOCC(COCCC(=O)NCCCNC(=O)CCCCO[C@H]2O[C@@H](CO)[C@@H](O)[C@@H](O)[C@@H]2NC(C)=O)(COCCC(=O)NCCCNC(=O)CCCCO[C@H]2O[C@@H](CO)[C@@H](O)[C@@H](O)[C@@H]2NC(C)=O)NC(=O)CCCCCCCCCCC(=O)NCCN2C(=O)C=CC2=O)O[C@@H](CO)[C@@H](O)[C@H]1O] |
| 1227 (I-170) | 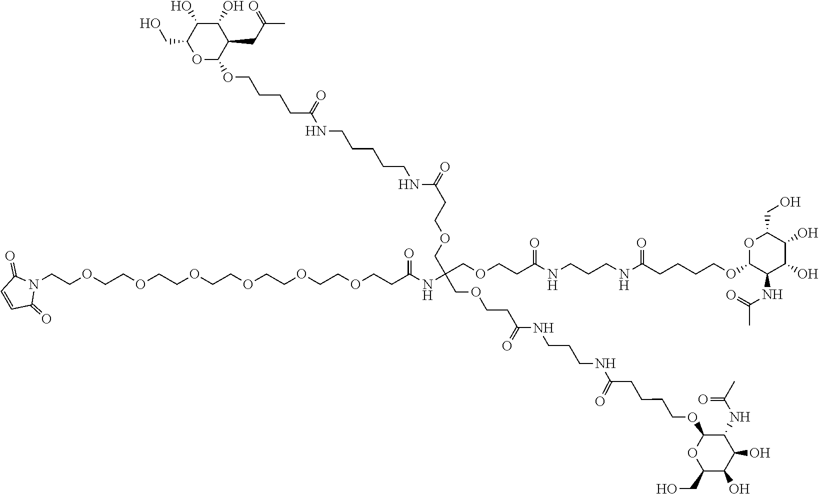 [SMILES: CC(=O)C[C@H]1[C@H](OCCCCC(=O)NCCCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCOCCOCCN2C(=O)C=CC2=O)O[C@H](CO)[C@H](O)[C@@H]1O] |
| 1228 | 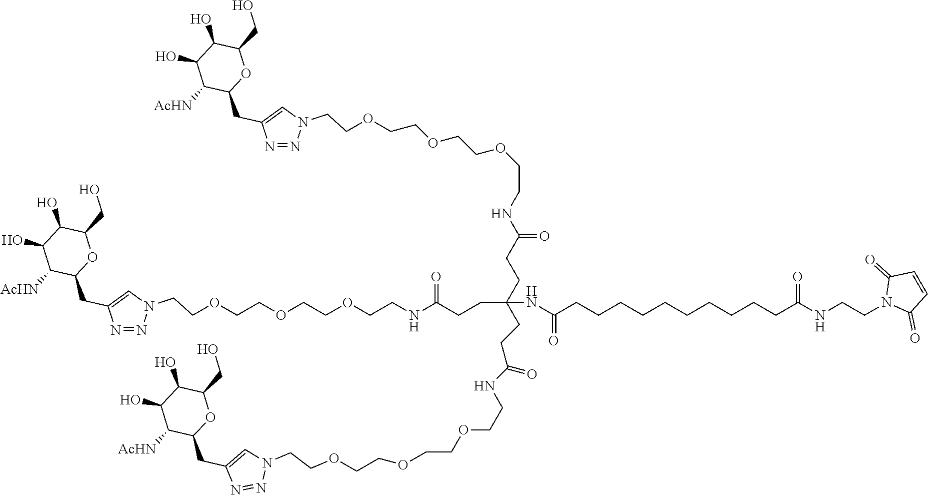 [SMILES: CC(=O)N[C@@H]1[C@@H](O)[C@@H](O)[C@@H](CO)O[C@H]1Cc1cn(CCOCCOCCOCCNC(=O)CCC(CCC(=O)NCCOCCOCCOCCn2cc(C[C@@H]3O[C@H](CO)[C@H](O)[C@H](O)[C@H]3NC(C)=O)nn2)(CCC(=O)NCCOCCOCCOCCn2cc(C[C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1228A | 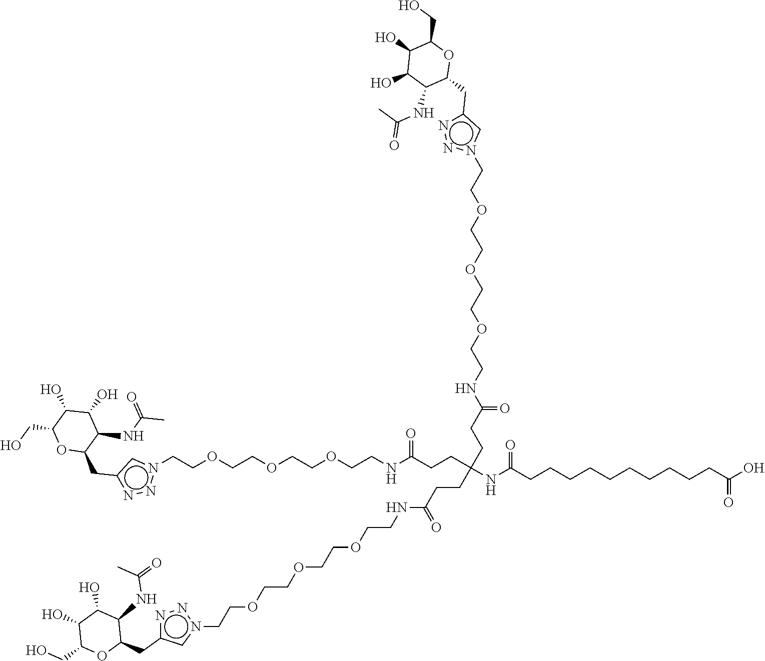 |
| 1228B | 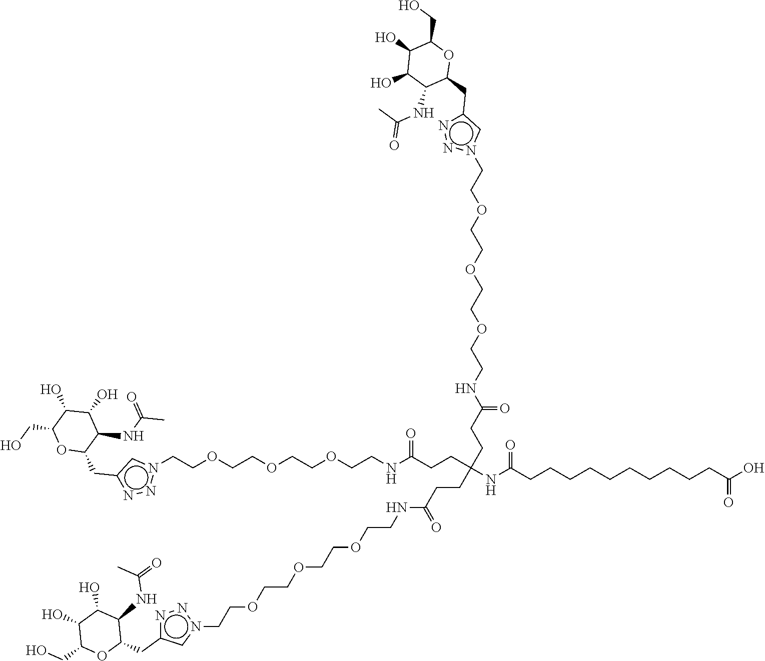 |
| 1229 | 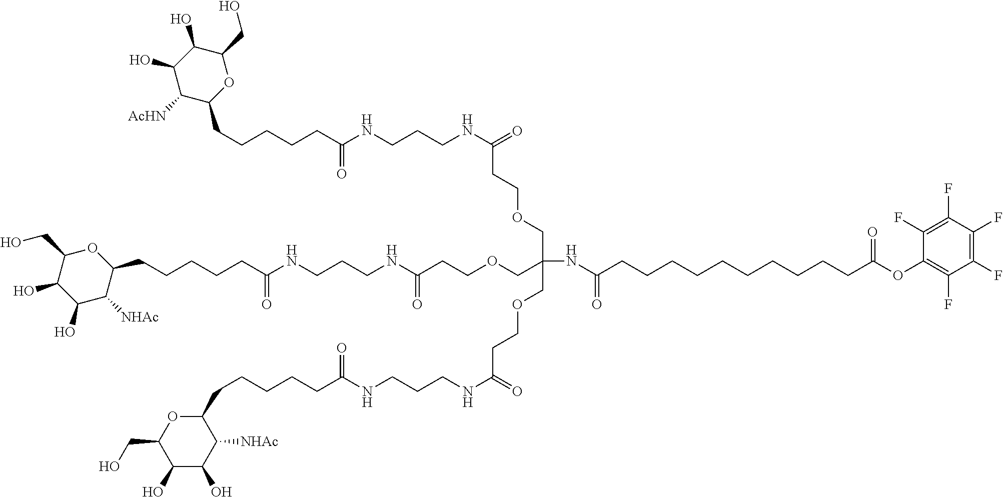 [SMILES: CC(=O)N[C@@H]1[C@@H](O)[C@@H](O)[C@@H](CO)O[C@H]1CCCCCC(=O)NCCCNC(=O)CCOCC(COCCC(=O)NCCCNC(=O)CCCCC[C@@H]1O[C@H](CO)[C@H](O)[C@H](O)[C@H]1NC(C)=O)(COCCC(=O)NCCCNC(=O)CCCCC[C@@H]1O[C@H](CO)[C@H](O)[C@H](O)[C@H]1NC(C)=O)NC(=O)CCCCCCCCCCC(=O)Oc1c(F)c(F)c(F)c(F)c1F] |
| 1230 | 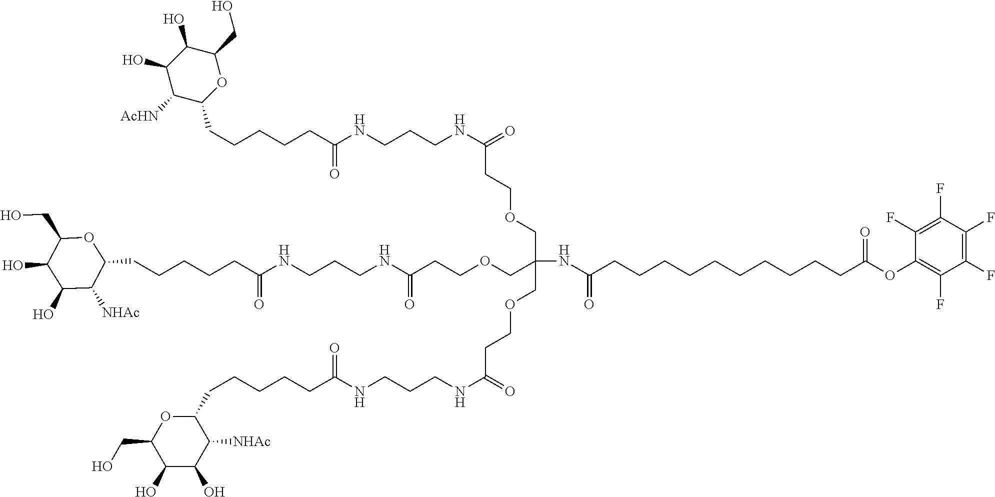 [SMILES: CC(=O)N[C@@H]1[C@@H](O)[C@@H](O)[C@@H](CO)O[C@@H]1CCCCCC(=O)NCCCNC(=O)CCOCC(COCCC(=O)NCCCNC(=O)CCCCC[C@H]1O[C@H](CO)[C@H](O)[C@H](O)[C@H]1NC(C)=O)(COCCC(=O)NCCCNC(=O)CCCCC[C@H]1O[C@H](CO)[C@H](O)[C@H](O)[C@H]1NC(C)=O)NC(=O)CCCCCCCCCCC(=O)Oc1c(F)c(F)c(F)c(F)c1F] |
| 1231 | 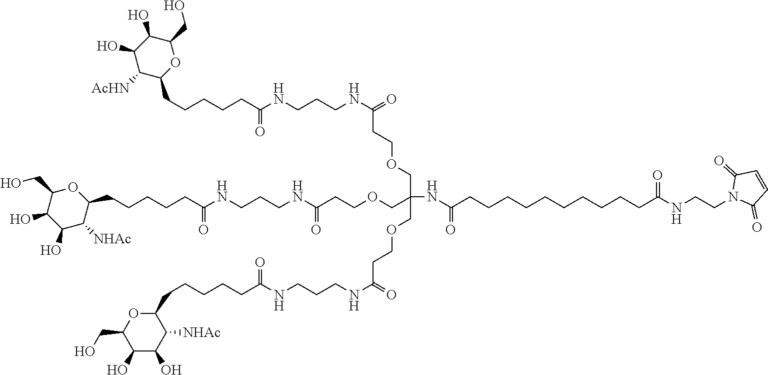 [SMILES: CC(=O)N[C@@H]1[C@@H](O)[C@@H](O)[C@@H](CO)O[C@H]1CCCCCC(=O)NCCCNC(=O)CCOCC(COCCC(=O)NCCCNC(=O)CCCCC[C@@H]1O[C@H](CO)[C@H](O)[C@H](O)[C@H]1NC(C)=O)(COCCC(=O)NCCCNC(=O)CCCCC[C@@H]1O[C@H](CO)[C@H](O)[C@H](O)[C@H]1NC(C)=O)NC(=O)CCCCCCCCCCC(=O)NCCN1C(=O)C=CC1=O] |
| 1232 | 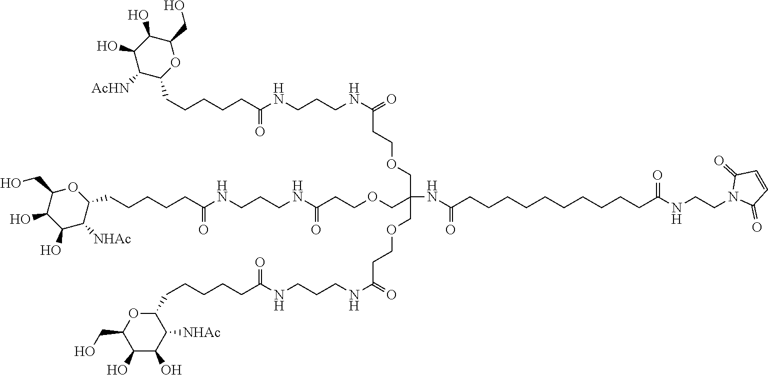 [SMILES: CC(=O)N[C@@H]1[C@@H](O)[C@@H](O)[C@@H](CO)O[C@@H]1CCCCCC(=O)NCCCNC(=O)CCOCC(COCCC(=O)NCCCNC(=O)CCCCC[C@H]1O[C@H](CO)[C@H](O)[C@H](O)[C@H]1NC(C)=O)(COCCC(=O)NCCCNC(=O)CCCCC[C@H]1O[C@H](CO)[C@H](O)[C@H](O)[C@H]1NC(C)=O)NC(=O)CCCCCCCCCCC(=O)NCCN1C(=O)C=CC1=O] |
| 1232A | 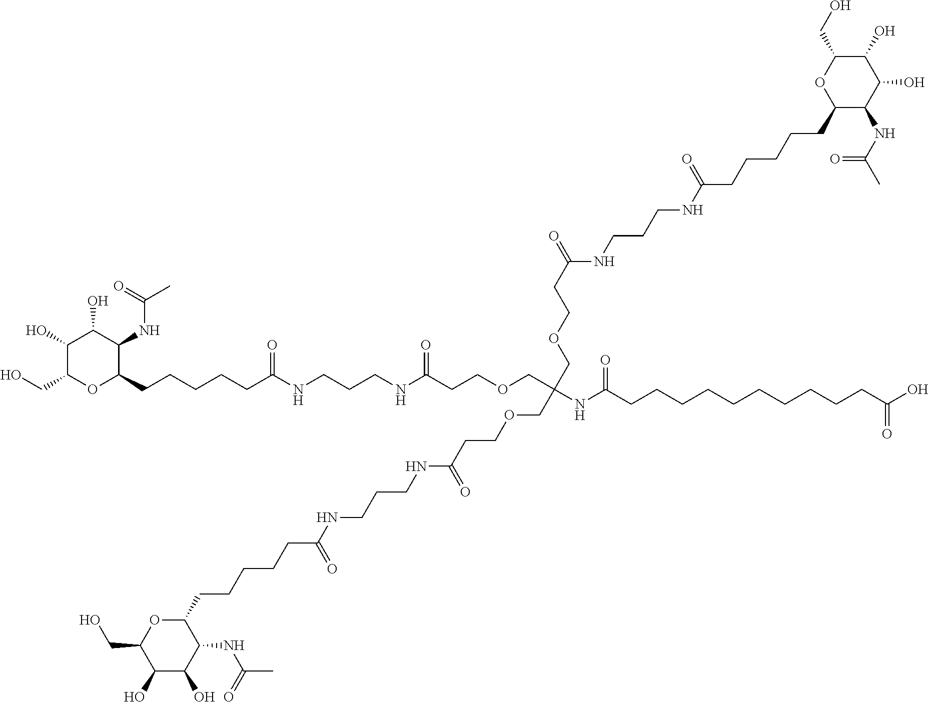 [SMILES: CC(=O)N[C@@H]1[C@@H](O)[C@@H](O)[C@@H](CO)O[C@@H]1CCCCCC(=O)NCCCNC(=O)CCOCC(COCCC(=O)NCCCNC(=O)CCCCC[C@H]1O[C@H](CO)[C@H](O)[C@H](O)[C@H]1NC(C)=O)(COCCC(=O)NCCCNC(=O)CCCCC[C@H]1O[C@H](CO)[C@H](O)[C@H](O)[C@H]1NC(C)=O)NC(=O)CCCCCCCCCCC(=O)O] |
| 1233 | 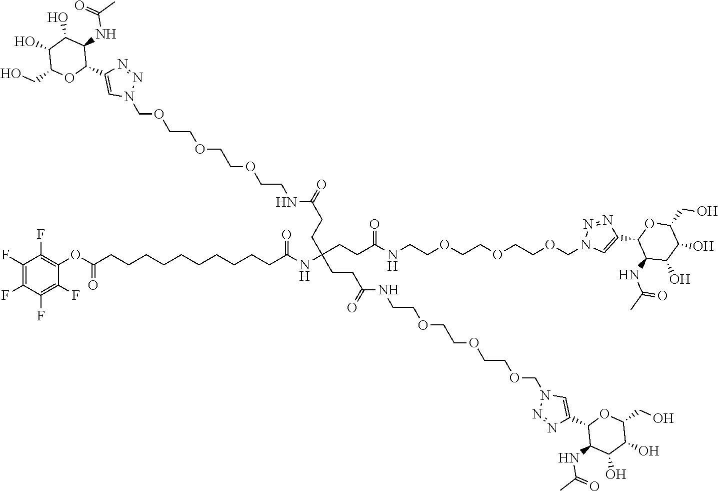 [SMILES: CC(=O)N[C@@H]1[C@@H](O)[C@@H](O)[C@@H](CO)O[C@H]1c1cn(COCCOCCOCCNC(=O)CCC(CCC(=O)NCCOCCOCCOCn2cc([C@@H]3O[C@H](CO)[C@H](O)[C@H](O)[C@H]3NC(C)=O)nn2)(CCC(=O)NCCOCCOCCOCn2cc([C@@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1233B | 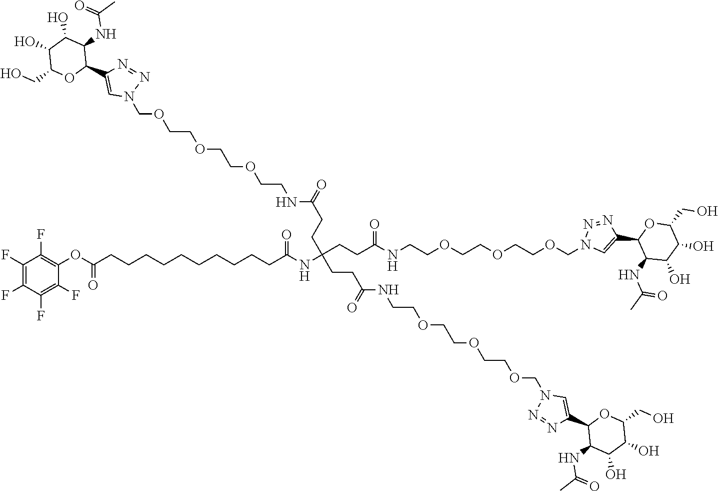 [SMILES: CC(=O)N[C@@H]1[C@@H](O)[C@@H](O)[C@@H](CO)O[C@@H]1c1cn(COCCOCCOCCNC(=O)CCC(CCC(=O)NCCOCCOCCOCn2cc([C@H]3O[C@H](CO)[C@H](O)[C@H](O)[C@H]3NC(C)=O)nn2)(CCC(=O)NCCOCCOCCOCn2cc([C@H]3O[C@H](CO)[C@H](O)[C@H](O)[C@H]3NC(C)=O)nn2)NC(=O)CCCCCCCCCCC(=O)Oc2c(F)c(F)c(F)c(F)c2F)nn1] |
| 1234 | 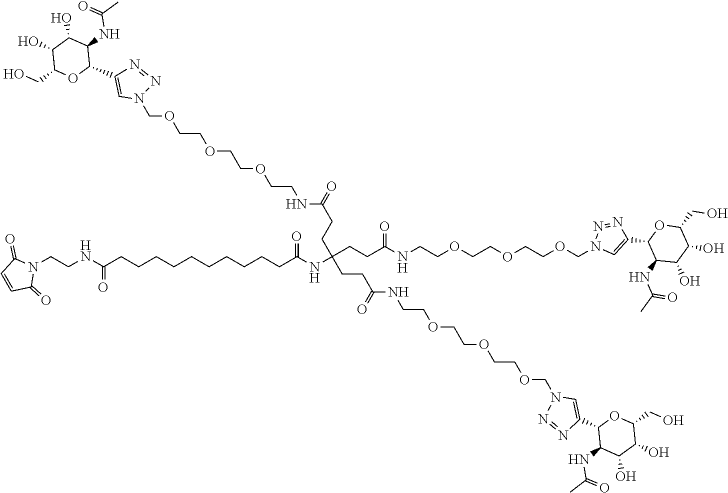 [SMILES: CC(=O)N[C@@H]1[C@@H](O)[C@@H](O)[C@@H](CO)O[C@H]1c1cn(COCCOCCOCCNC(=O)CCC(CCC(=O)NCCOCCOCCOCn2cc([C@@H]3O[C@H](CO)[C@H](O)[C@H](O)[C@H]3NC(C)=O)nn2)(CCC(=O)NCCOCCOCCOCn2cc([C@@H]3O[C@H](CO)[C@H](O)[C@H](O)[C@H]3NC(C)=O)nn2)NC(=O)CCCCCCCCCCC(=O)NCCN2C(=O)C=CC2=O)nn1] |
| 1234B | 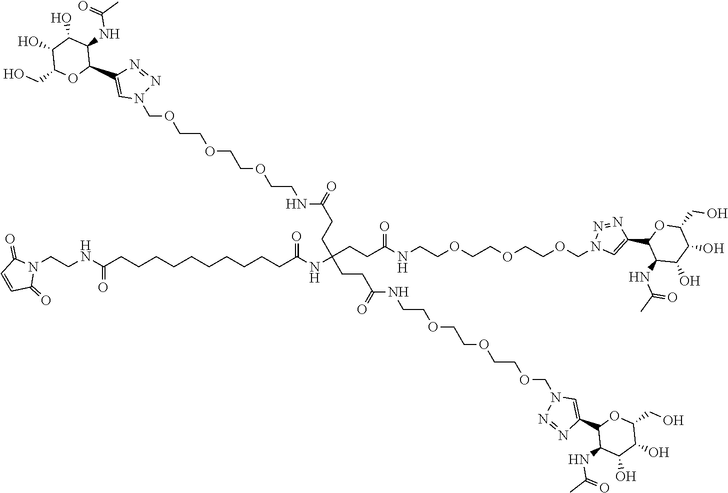 |
| 1243 | 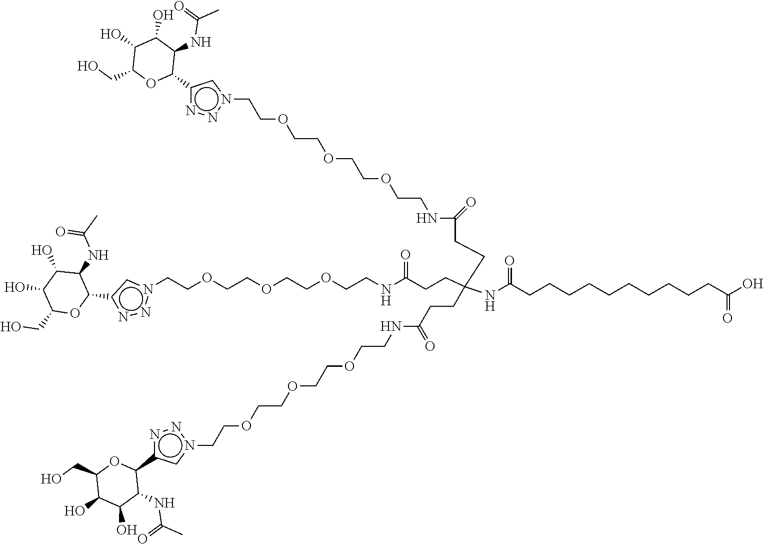 |
| 1244 | 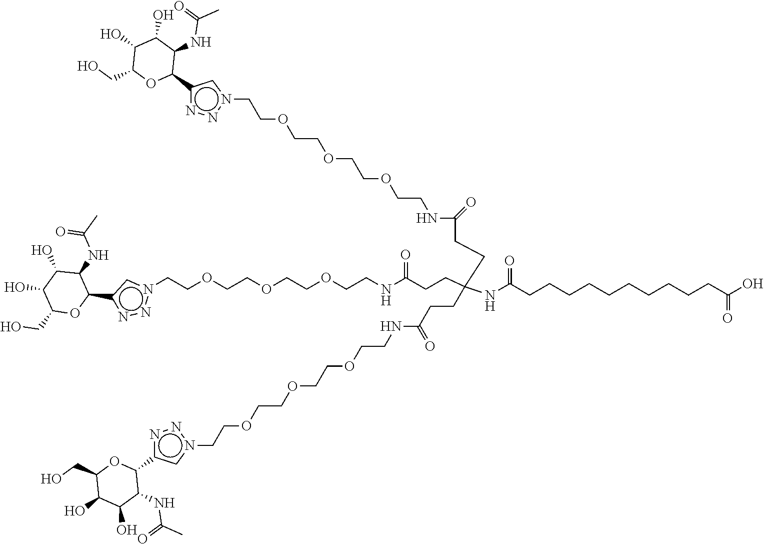 |
| 1245 | 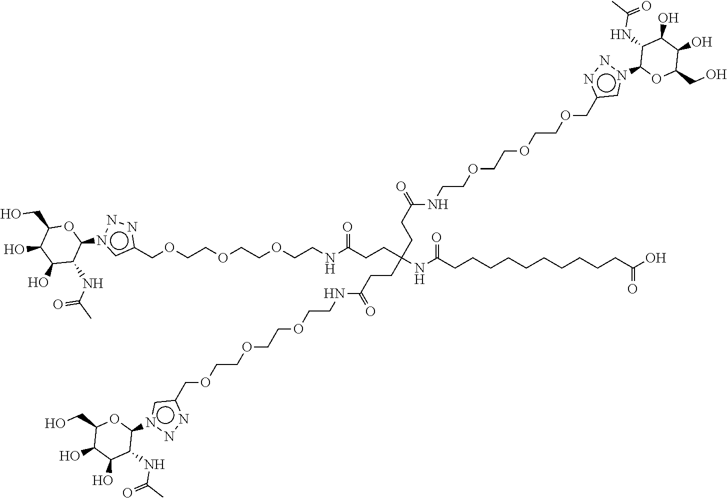 |
| 1246 | 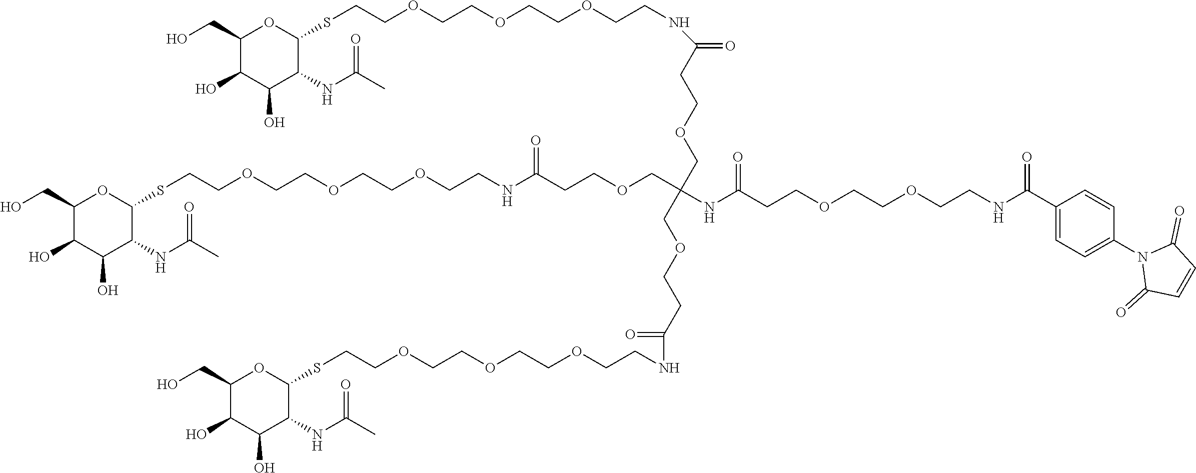 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCNC(=O)c1ccc(N2C(=O)C=CC2=O)cc1] |
| 1247 | 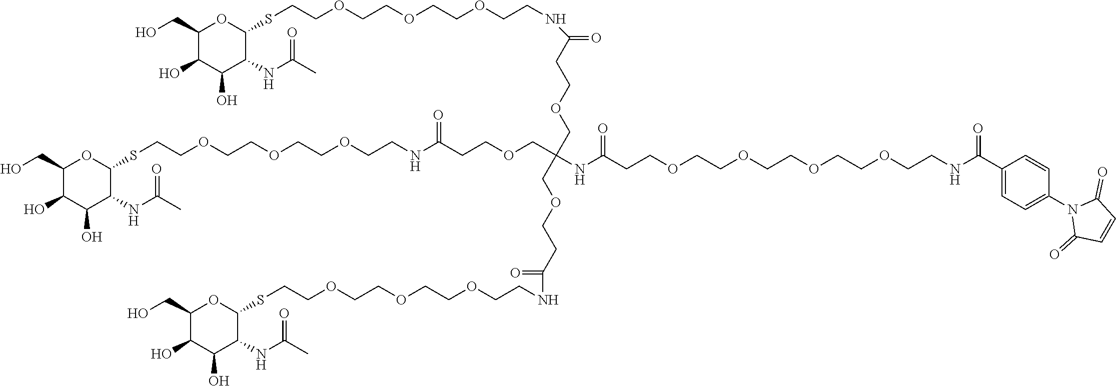 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1248 | 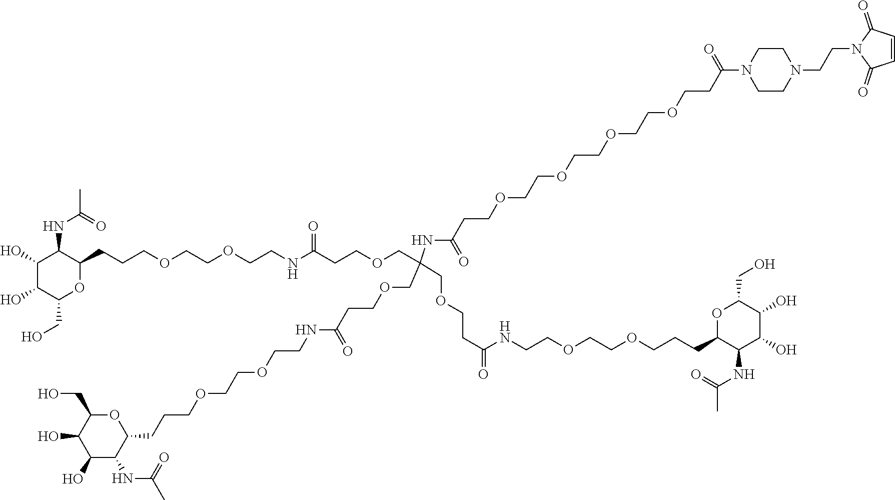 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCC(=O)N1CCN(CCN2C(=O)C=CC2=O)CC1] |
| 1249 | 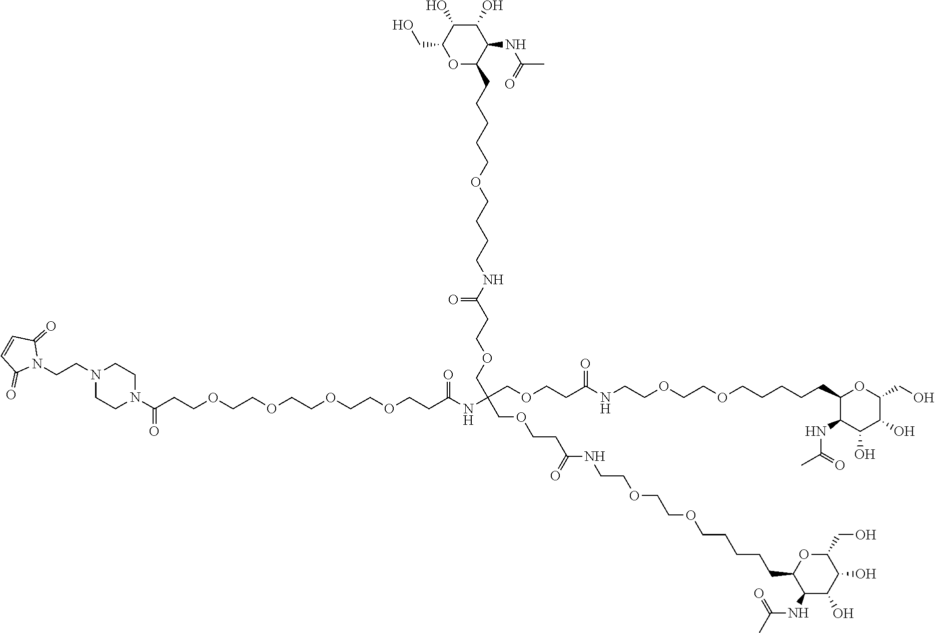 [SMILES: CC(=O)N[C@@H]1[C@@H](O)[C@@H](O)[C@@H](CO)O[C@@H]1CCCCCOCCCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCC(=O)N1CCN(CCN2C(=O)C=CC2=O)CC1] |
| 1250 | 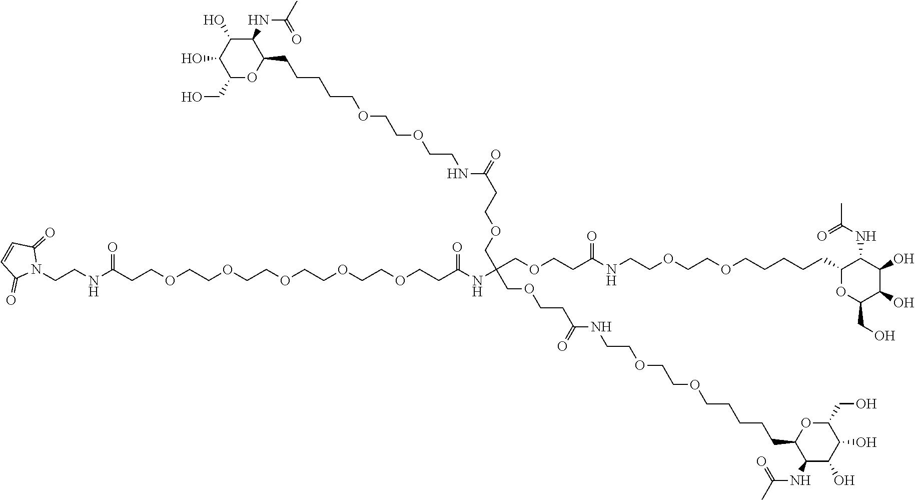 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCOCCC(=O)NCCN1C(=O)C=CC1=O] |
| 1251 | 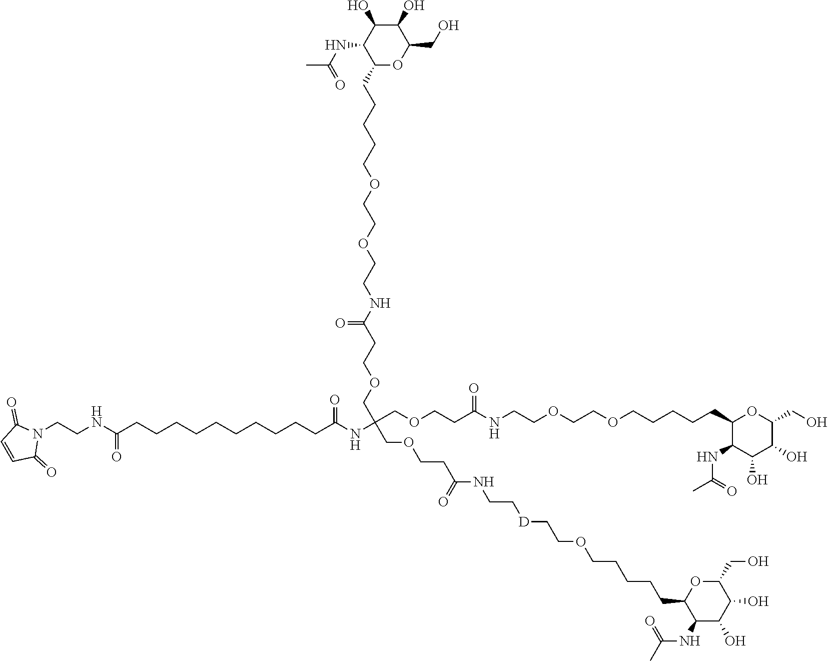 |
| 1252 | 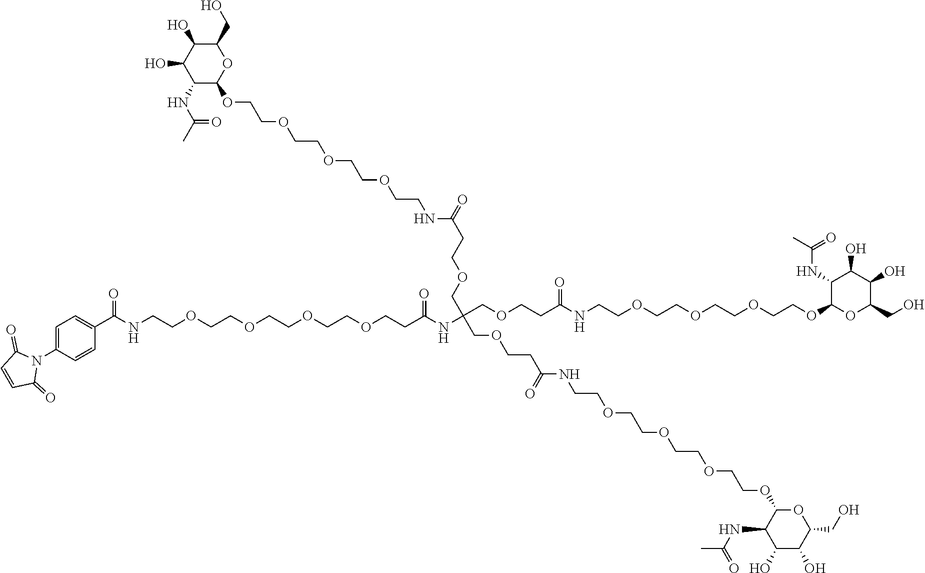 [SMILES: CC(=O)N[C@H]1[C@H](OCCOCCOCCOCCNC(=O)CCOCC(COCCC(=O)NCCOCCOCCOCCO[C@@H]2O[C@H](CO)[C@H](O)[C@H](O)[C@H]2NC(C)=O)(COCCC(=O)NCCOCCOCCOCCO[C@@H]2O[C@H](CO)[C@H](O)[C@H](O)[C@H]2NC(C)=O)NC(=O)CCOCCOCCOCCOCCNC(=O)c2ccc(N3C(=O)C=CC3=O)cc2)O[C@H](CO)[C@H](O)[C@@H]1O] |
| 1253 | 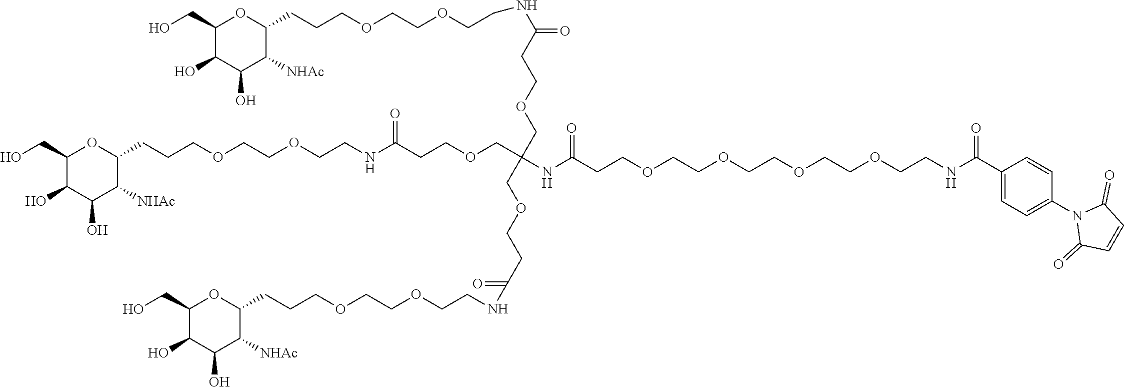 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1253A | 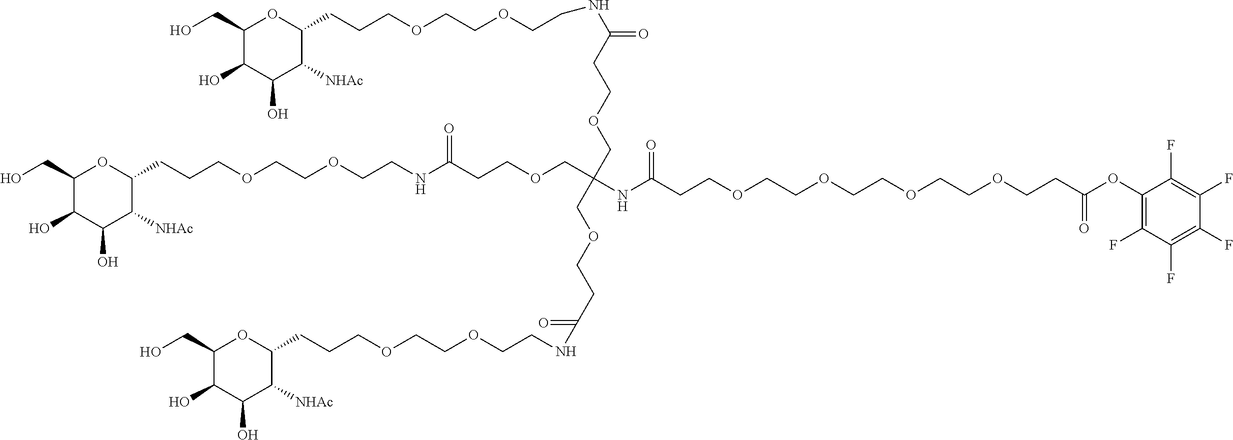 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 1254 | 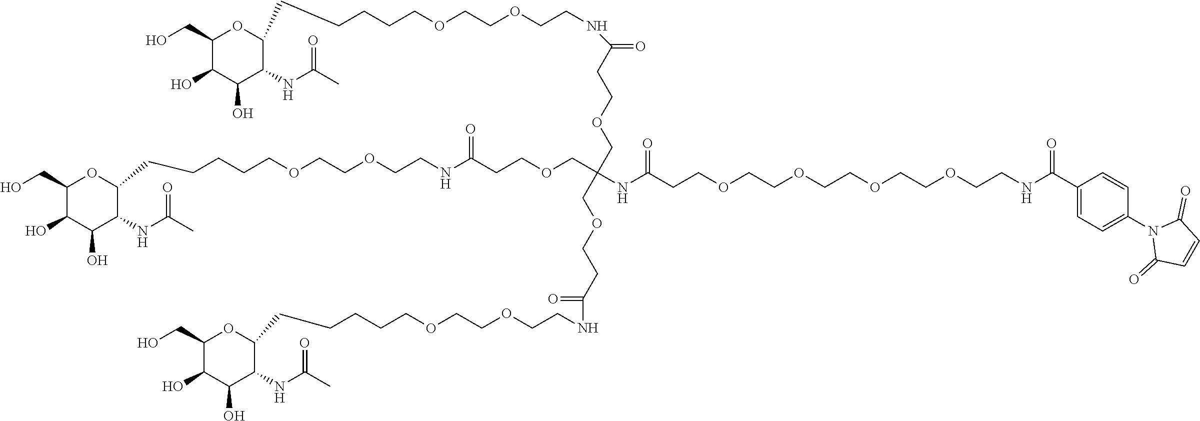 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1255 | 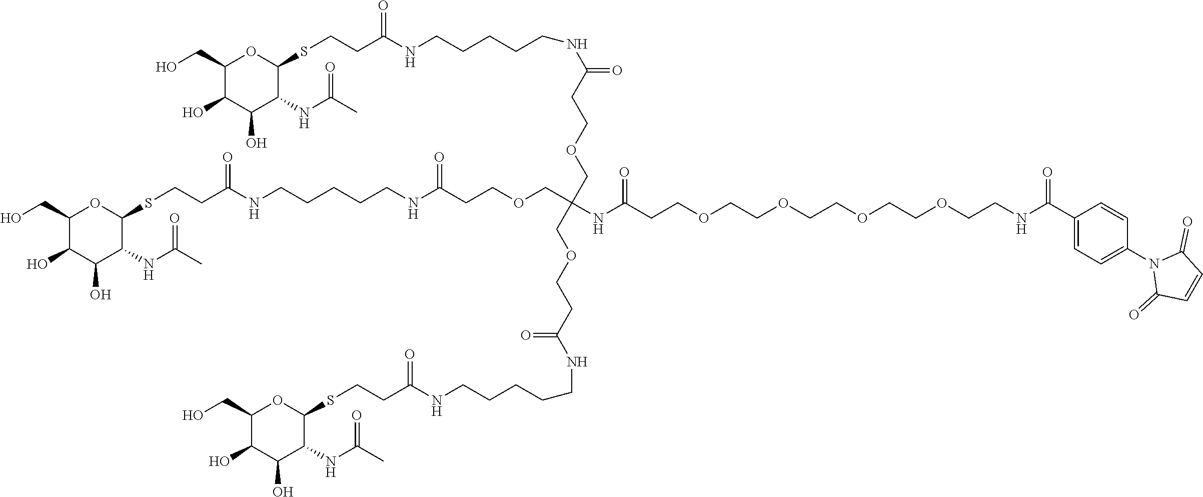 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1256 | 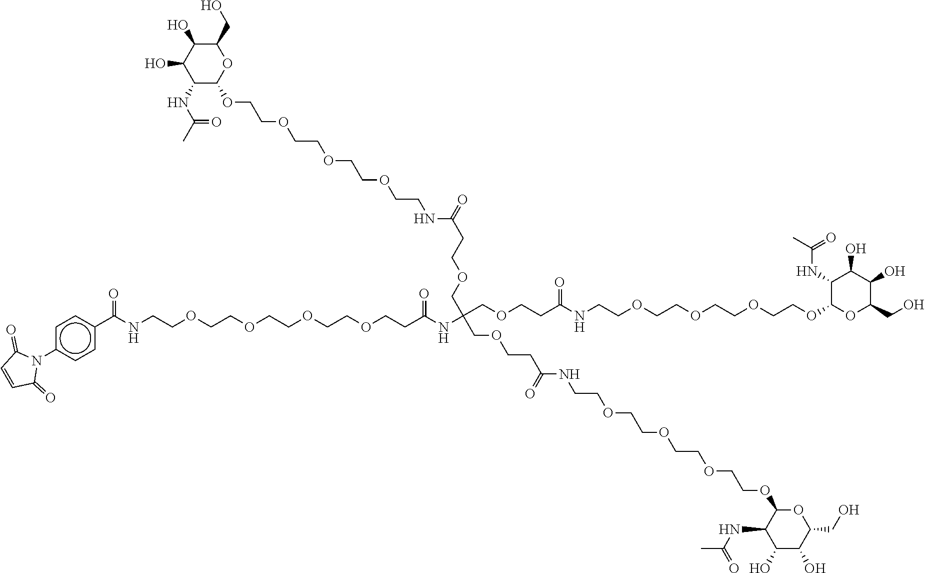 |
| 1257 | 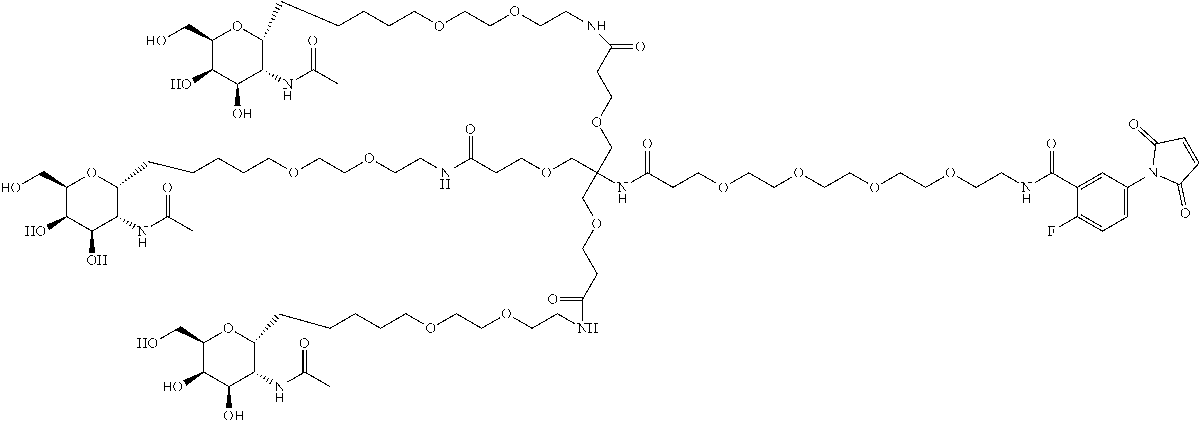 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1cc(N2C(=O)C=CC2=O)ccc1F] |
| 1258 | 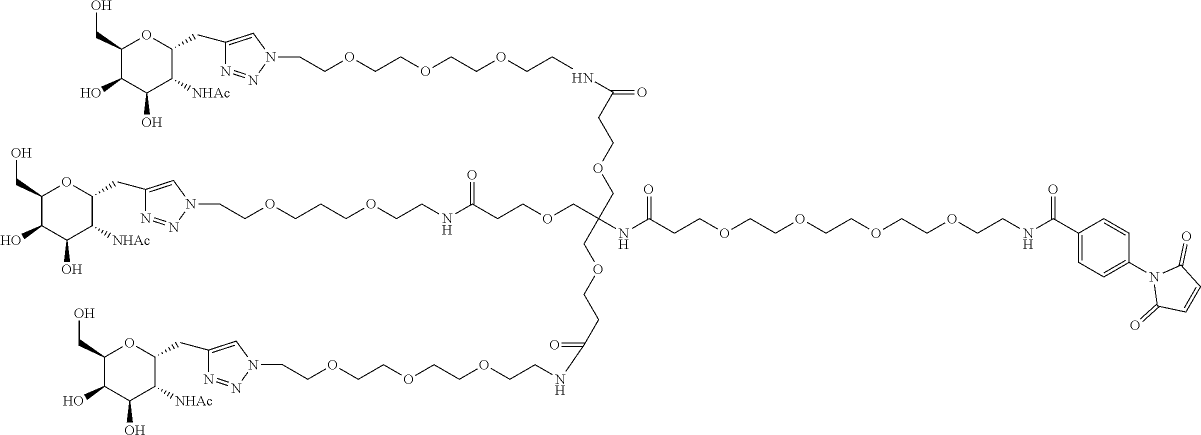 [SMILES: CC(=O)N[C@@H]1[C@@H](O)[C@@H](O)[C@@H](CO)O[C@@H]1Cc1cn(CCOCCCOCCNC(=O)CCOCC(COCCC(=O)NCCOCCOCCOCCn2cc(C[C@H]3O[C@H](CO)[C@H](O)[C@H](O)[C@H]3NC(C)=O)nn2)(COCCC(=O)NCCOCCOCCOCCn2cc(C[C@H]3O[C@H](CO)[C@H](O)[C@H](O)[C@H]3NC(C)=O)nn2)NC(=O)CCOCCOCCOCCOCCNC(=O)c2ccc(N3C(=O)C=CC3=O)cc2)nn1] |
| 1259 | 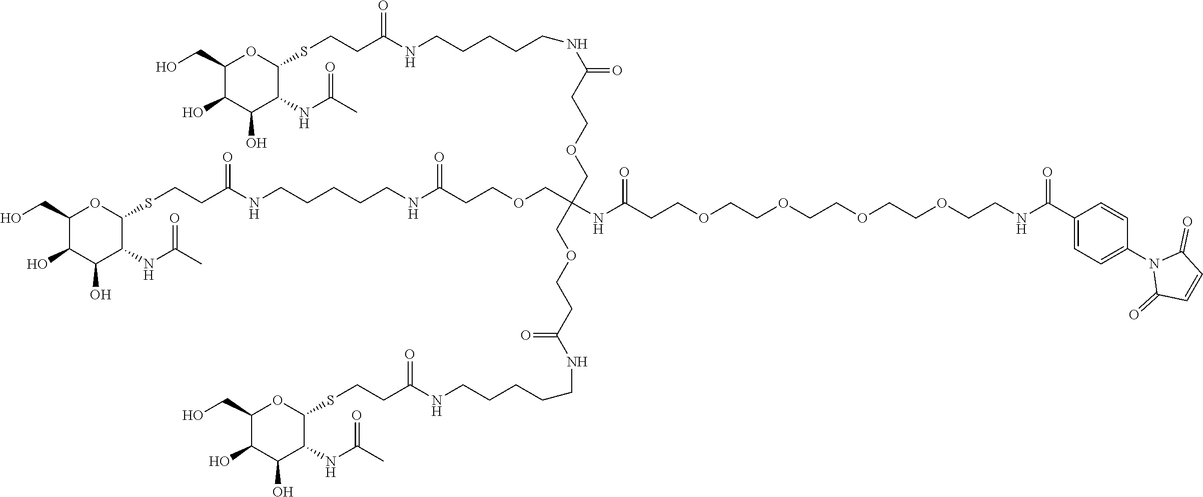 [SMILES: CC(=O)N[C@@H]1[C@@H](O)[C@@H](O)[C@@H](CO)O[C@@H]1SCCC(=O)NCCCCCNC(=O)CCOCC(COCCC(=O)NCCCCCNC(=O)CCS[C@H]1O[C@H](CO)[C@H](O)[C@H](O)[C@H]1NC(C)=O)(COCCC(=O)NCCCCCNC(=O)CCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 1260 (32-11) | 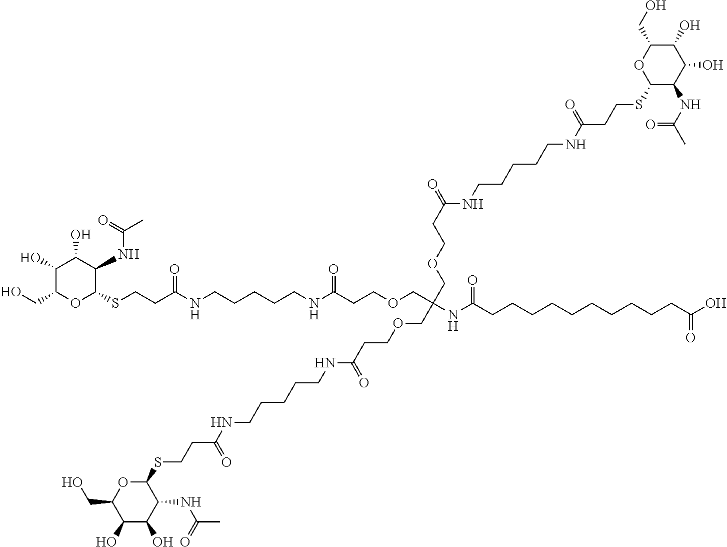 [SMILES: CC(=O)N[C@@H]1[C@@H](O)[C@@H](O)[C@@H](CO)O[C@H]1SCCC(=O)NCCCCCNC(=O)CCOCC(COCCC(=O)NCCCCCNC(=O)CCS[C@@H]1O[C@H](CO)[C@H](O)[C@H](O)[C@H]1NC(C)=O)(COCCC(=O)NCCCCCNC(=O)CCS[C@@H]1O[C@H](CO)[C@H](O)[C@H](O)[C@H]1NC(C)=O)NC(=O)CCCCCCCCCCC(=O)O] |
| 1254 | 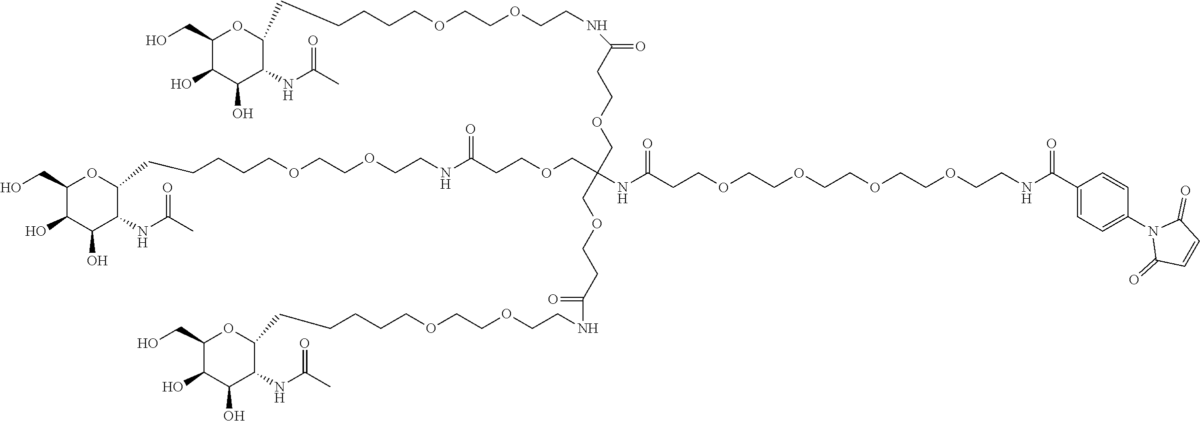 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2346 | 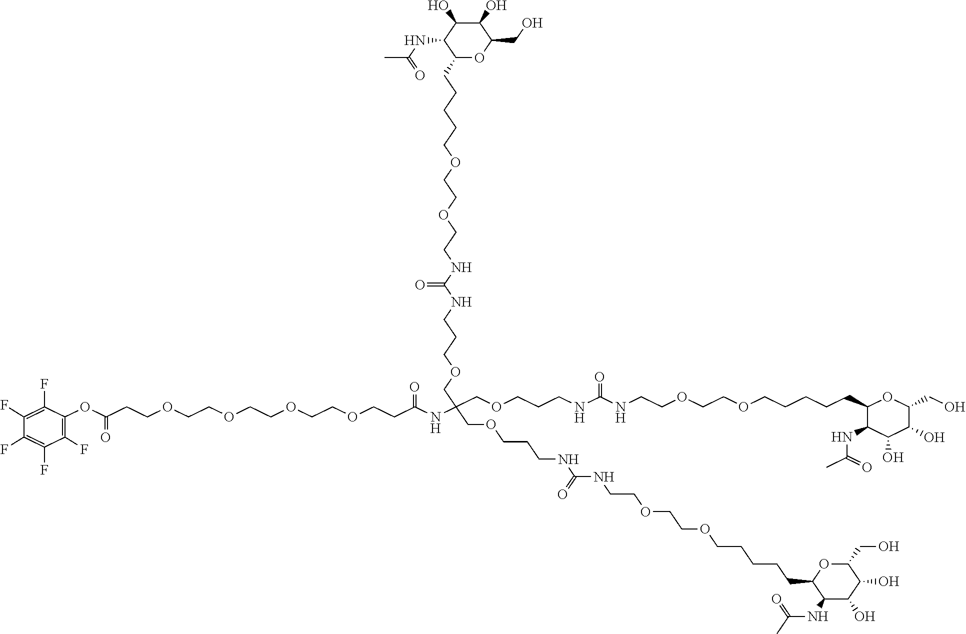 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)(COCCCNC(=O)NCCOCCOCCCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2378 | 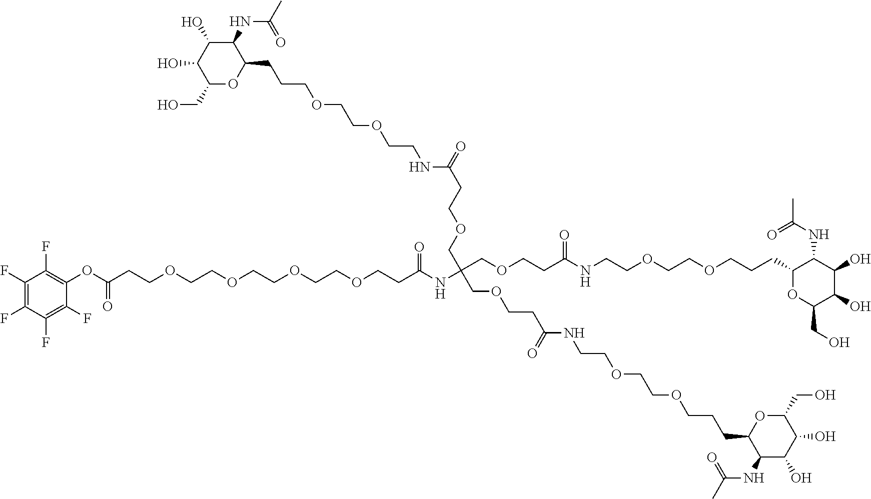 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |

TABLE 16
Multivalent ASGPR binding compounds having chemoselective ligation group and X
group of formula (Ic)

| # | Structure |
|---|---|
| 1301 (I- 110) | 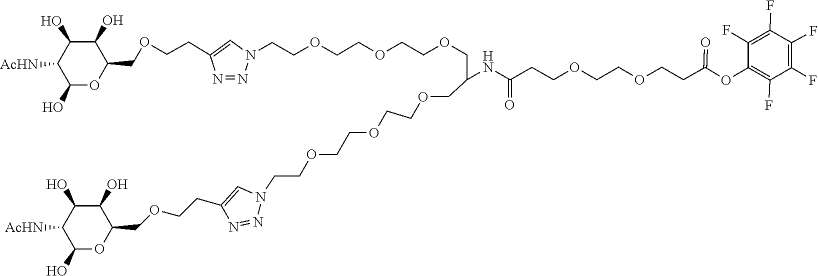 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |

Trimeric ligands

| | |
|---|---|
| 1302 (I- 108) | 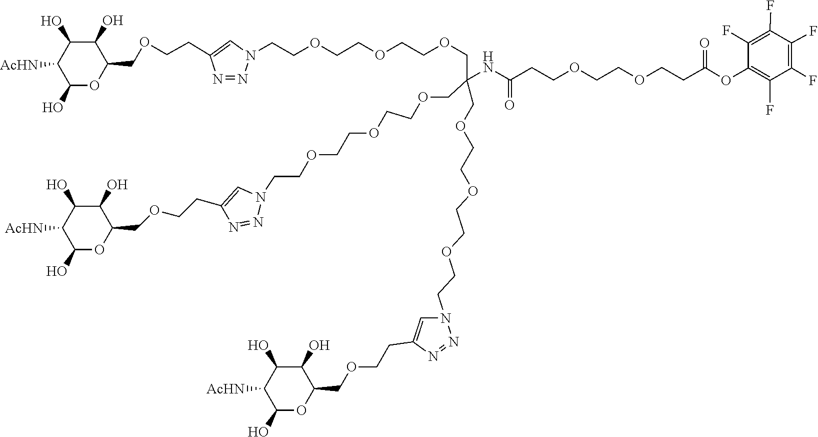 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |
| 1303 (I- 136) | 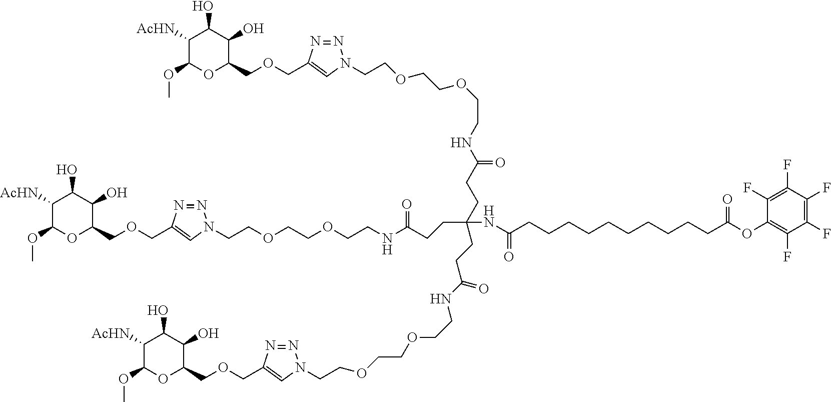 [SMILES: CO[C@@H]1O[C@H](COCc2cn(CCOCCOCCNC(=O)CCC(CCC(=O)NCCOCCOCCn3cc(COC[C@H]4O[C@@H](OC)[C@H](NC(C)=O)[C@@H](O)[C@H]4O)nn3)(CCC(=O)NCCOCCOCCn3cc(COC[C@H]4O[C@@H](OC)[C@H](NC(C)=O)[C@@H](O)[C@H]4O)nn3)NC(=O)CCCCCCCCCCC(=O)Oc3c(F)c(F)c(F)c(F)c3F)nn2)[C@H](O)[C@H](O)[C@H]1NC(C)=O] |
| 1304 (I- 152) | 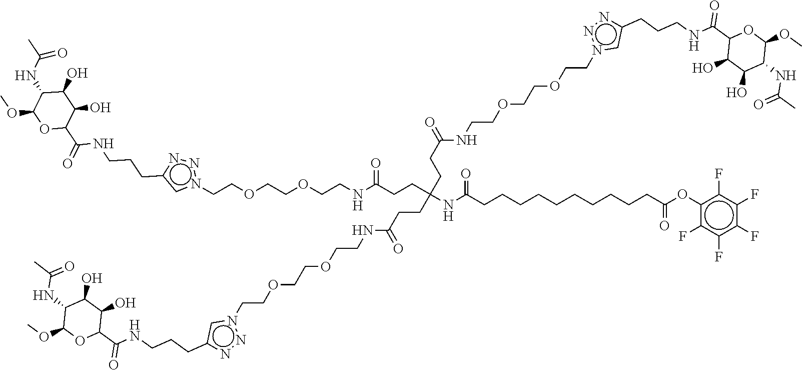 |

TABLE 17
Multivalent ASGPR binding compounds having chemoselective ligation group and X
group of formula (Id)

| # | Structure |
|---|---|
| 1401 (I- 153) | 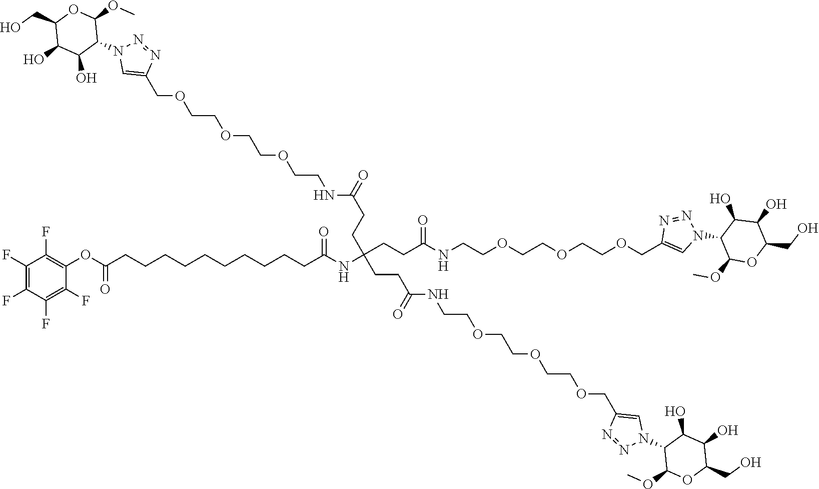 [SMILES: CO[C@@H]1O[C@H](CO)[C@H](O)[C@H](O)[C@H]1n1cc(COCCOCCOCCNC(=O)CCC(CCC(=O)NCCOCCOCCOCc2cn([C@H]3[C@H](OC)O[C@H](CO)[C@H](O)[C@@H]3O)nn2)(CCC(=O)NCCOCCOCCOCc2cn([C@H]3[C@H](OC)O[C@H](CO)[C@H](O)[C@@H]3O)nn2)NC(=O)CCCCCCCCCCC(=O)Oc2c(F)c(F)c(F)c(F)c2F)nn1] |
| 1402 (I- 154) | 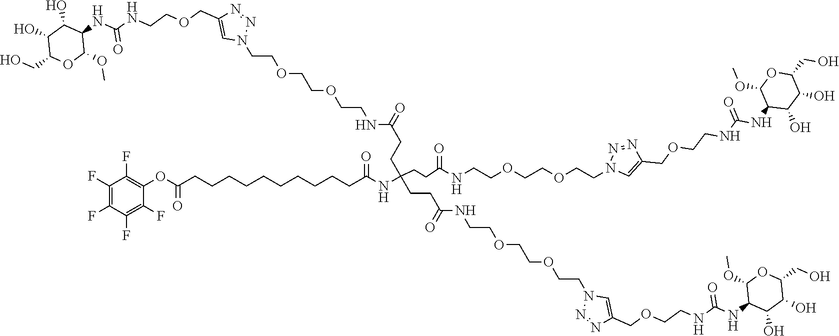 [SMILES: CO[C@@H]1O[C@H](CO)[C@H](O)[C@H](O)[C@H]1NC(=O)NCCOCc1cn(CCOCCOCCNC(=O)CCC(CCC(=O)NCCOCCOCCn2cc(COCCNC(=O)N[C@H]3[C@H](OC)O[C@H](CO)[C@H](O)[C@@H]3O)nn2)(CCC(=O)NCCOCCOCCn2cc(COCCNC(=O)N[C@H]3[C@H](OC)O[C@H](CO)[C@H](O)[C@@H]3O)nn2)NC(=O)CCCCCCCCCCC(=O)Oc2c(F)c(F)c(F)c(F)c2F)nn1] |
| 1403 (I- 155) | 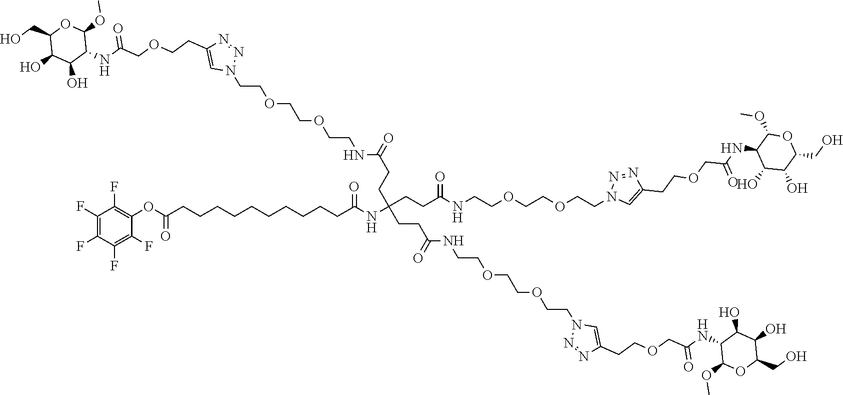 [SMILES: CO[C@@H]1O[C@H](CO)[C@H](O)[C@H](O)[C@H]1NC(=O)COCCc1cn(CCOCCOCCNC(=O)CCC(CCC(=O)NCCOCCOCCn2cc(CCOCC(=O)N[C@H]3[C@H](OC)O[C@H](CO)[C@H](O)[C@@H]3O)nn2)(CCC(=O)NCCOCCOCCn2cc(CCOCC(=O)N[C@H]3[C@H](OC)O[C@H](CO)[C@H](O)[C@@H]3O)nn2)NC(=O)CCCCCCCCCCC(=O)Oc2c(F)c(F)c(F)c(F)c2F)nn1] |

The present disclosure is meant to encompass stereoisomers of any one of the compounds described herein. In some instance, the compound includes an enantiomer of the D-N-acetylgalactosamine (GalNAc), or an analog or derivative of GalNAc.

### Other Exemplary Compounds

Table 18 illustrates exemplary ASGPR binding compounds of this disclosure that include a binding moiety, or a precursor thereof.

TABLE 18
Multivalent ASGPR binding compounds having protein targeting group

| # | Structure |
|---|---|
| 1404 (I- 156) | 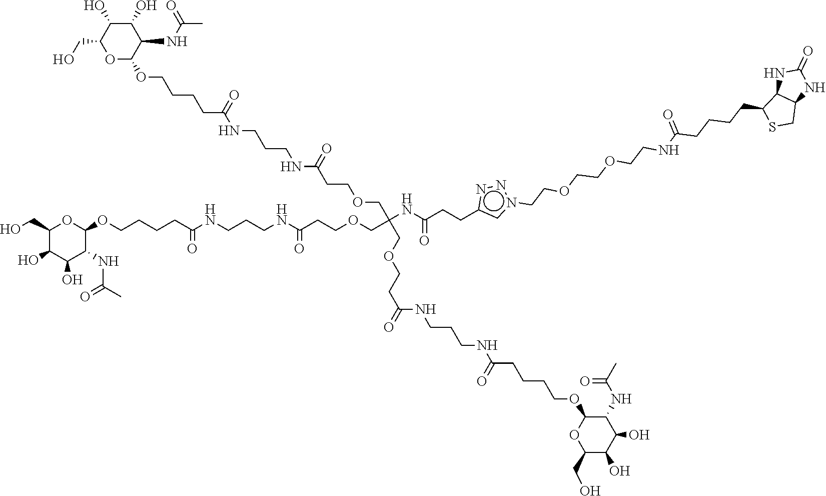 |
| 1405 (I- 139) | 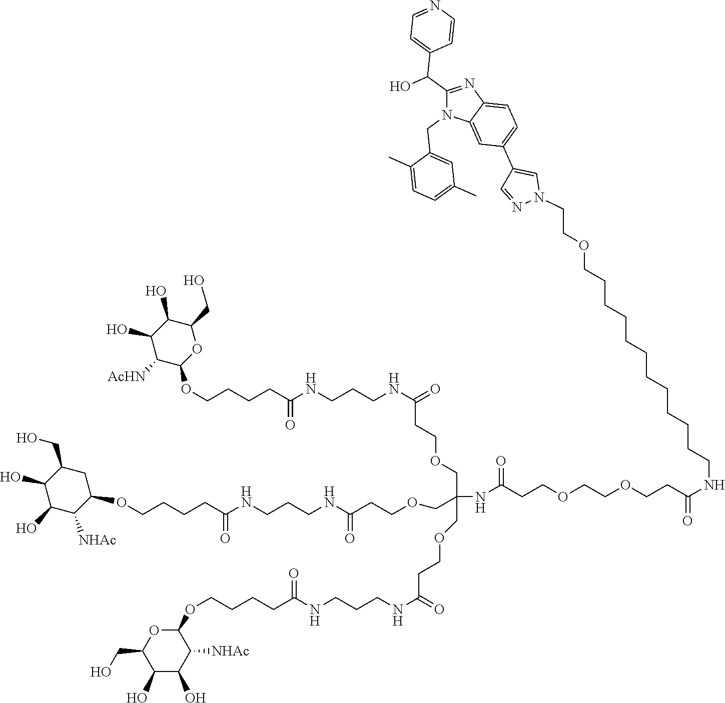 [SMILES: CC(=O)N[C@@H]1[C@@H](O)[C@@H](O)[C@@H](CO)C[C@H]1OCCCCC(=O)NCCCNC(=O)CCOCC(COCCC(=O)NCCCNC(=O)CCCCO[C@@H]1O[C@H](CO)[C@H](O)[C@H](O)[C@H]1NC(C)=O)(COCCC(=O)NCCCNC(=O)CCCCO[C@@H]1O[C@H](CO)[C@H](O)[C@H](O)[C@H]1NC(C)=O)NC(=O)CCOCCOCCC(=O)NCCCCCCCCCCCCOCCn1cc(-c2ccc3nc(C(O)c4ccncc4)n(Cc4cc(C)ccc4C)c3c2)cn1] |
| 1406 (I- 142) | 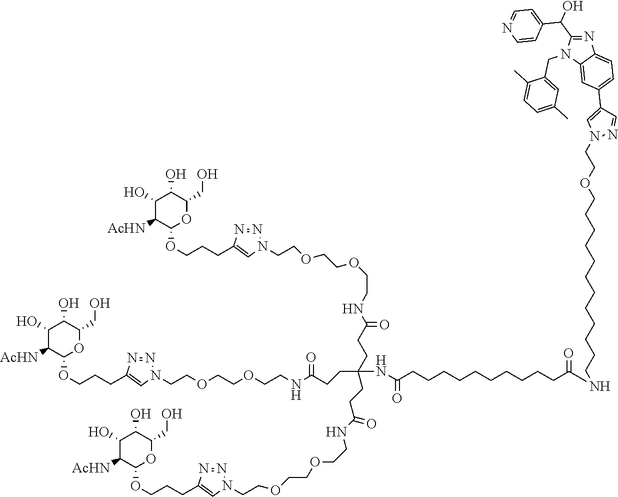 [SMILES: CC(=O)N[C@@H]1[C@@H](OCCCc2cn(CCOCCOCCNC(=O)CCC(CCC(=O)NCCOCCOCCn3cc(CCCO[C@H]4O[C@@H](CO)[C@@H](O)[C@@H](O)[C@@H]4NC(C)=O)nn3)(CCC(=O)NCCOCCOCCn3cc(CCCO[C@H]4O[C@@H](CO)[C@@H](O)[C@@H](O)[C@@H]4NC(C)=O)nn3)NC(=O)CCCCCCCCCCC(=O)NCCCCCCCCCCCCOCCn3cc(-c4ccc5nc(C(O)c6ccncc6)n(Cc6cc(C)ccc6C)c5c4)cn3)nn2)O[C@@H](CO)[C@@H](O)[C@H]1O] |

Table 19 illustrates exemplary trivalent ASGPR binding intermediate compounds of this disclosure including X groups of formula (Ie).

TABLE 19
Multivalent ASGPR binding compounds including X groups of formula (Ie)

| # | Structure |
|---|---|
| 1901 (I- 171) | 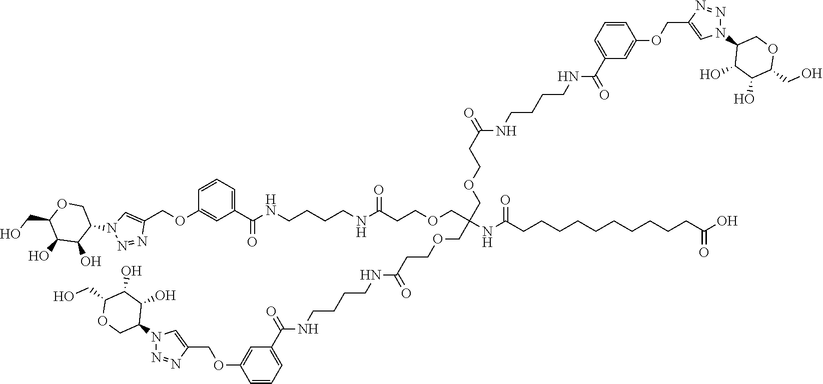 [SMILES: O=C(O)CCCCCCCCCCC(=O)NC(COCCC(=O)NCCCCNC(=O)c1cccc(OCc2cn([C@H]3CO[C@H](CO)[C@H](O)[C@@H]3O)nn2)c1)(COCCC(=O)NCCCCNC(=O)c1cccc(OCc2cn([C@H]3CO[C@H](CO)[C@H](O)[C@@H]3O)nn2)c1)COCCC(=O)NCCCCNC(=O)c1cccc(OCc2cn([C@H]3CO[C@H](CO)[C@H](O)[C@@H]3O)nn2)c1] |
| 1902 (I- 172) | 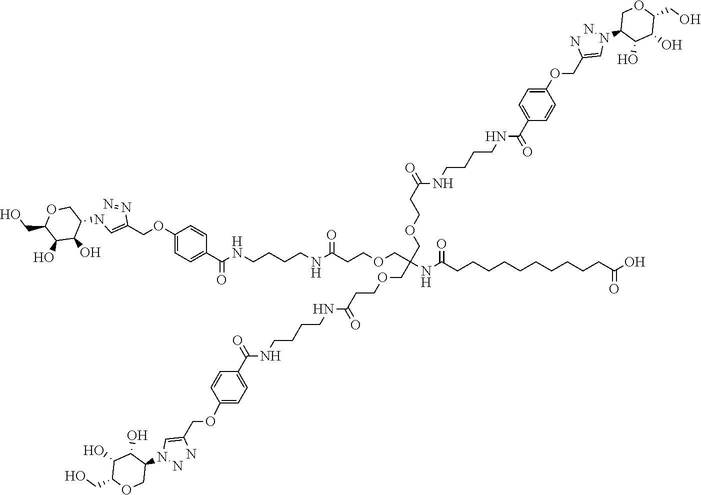 [SMILES: O=C(O)CCCCCCCCCCC(=O)NC(COCCC(=O)NCCCCNC(=O)c1ccc(OCc2cn([C@H]3CO[C@H](CO)[C@H](O)[C@@H]3O)nn2)cc1)(COCCC(=O)NCCCCNC(=O)c1ccc(OCc2cn([C@H]3CO[C@H](CO)[C@H](O)[C@@H]3O)nn2)cc1)COCCC(=O)NCCCCNC(=O)c1ccc(OCc2cn([C@H]3CO[C@H](CO)[C@H](O)[C@@H]3O)nn2)cc1] |
| 1904 | 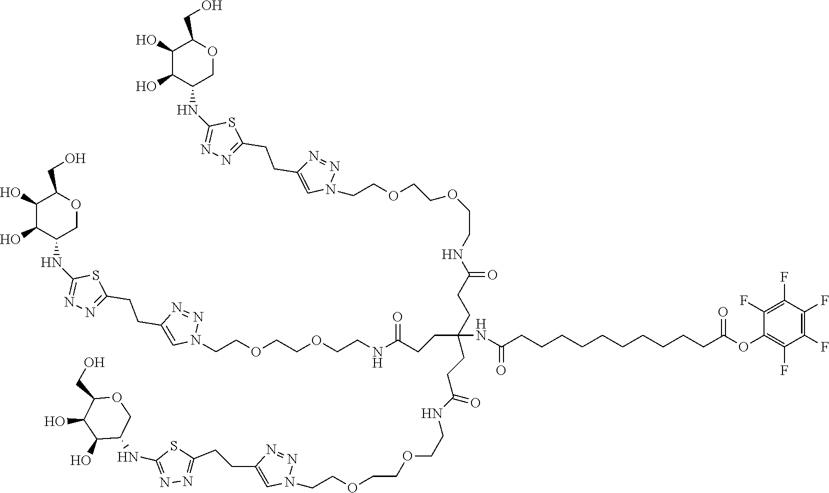 [SMILES: O=C(CCC(CCC(=O)NCCOCCOCCn1cc(CCc2nnc(N[C@H]3CO[C@H](CO)[C@H](O)[C@@H]3O)s2)nn1)(CCC(=O)NCCOCCOCCn1cc(CCc2nnc(N[C@H]3CO[C@H](CO)[C@H](O)[C@@H]3O)s2)nn1)NC(=O)CCCCCCCCCCC(=O)Oc1c(F)c(F)c(F)c(F)c1F)NCCOCCOCCn1cc(CCc2nnc(N[C@H]3CO[C@H](CO)[C@H](O)[C@@H]3O)s2)nn1] |
| 1905 | 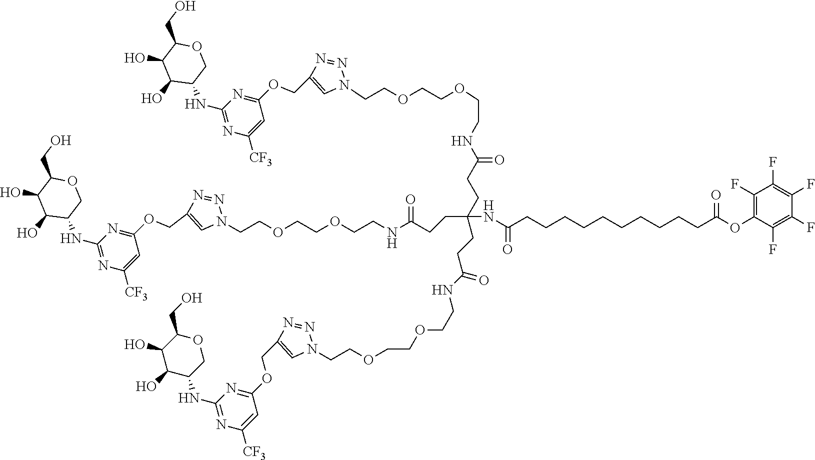 [SMILES: O=C(CCC(CCC(=O)NCCOCCOCCn1cc(COc2cc(C(F)(F)F)nc(N[C@H]3CO[C@H](CO)[C@H](O)[C@@H]3O)n2)nn1)(CCC(=O)NCCOCCOCCn1cc(COc2cc(C(F)(F)F)nc(N[C@H]3CO[C@H](CO)[C@H](O)[C@@H]3O)n2)nn1)NC(=O)CCCCCCCCCCC(=O)Oc1c(F)c(F)c(F)c(F)c1F)NCCOCCOCCn1cc(COc2cc(C(F)(F)F)nc(N[C@H]3CO[C@H](CO)[C@H](O)[C@@H]3O)n2)nn1] |
| 1905A | 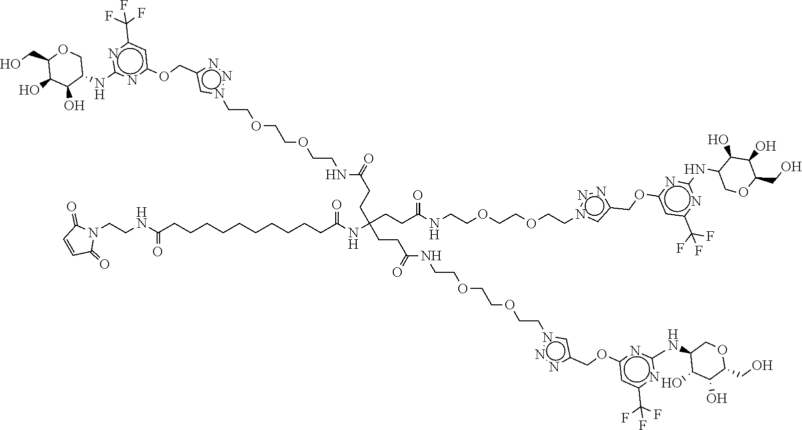 |
| 1906 | 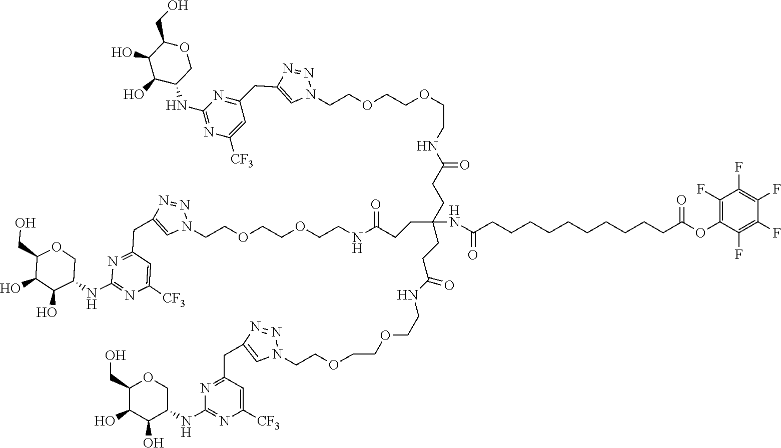 [SMILES: O=C(CCC(CCC(=O)NCCOCCOCCn1cc(Cc2cc(C(F)(F)F)nc(N[C@H]3CO[C@H](CO)[C@H](O)[C@@H]3O)n2)nn1)(CCC(=O)NCCOCCOCCn1cc(Cc2cc(C(F)(F)F)nc(N[C@H]3CO[C@H](CO)[C@H](O)[C@@H]3O)n2)nn1)NC(=O)CCCCCCCCCCC(=O)Oc1c(F)c(F)c(F)c(F)c1F)NCCOCCOCCn1cc(Cc2cc(C(F)(F)F)nc(N[C@H]3CO[C@H](CO)[C@H](O)[C@@H]3O)n2)nn1] |
| 1907 | 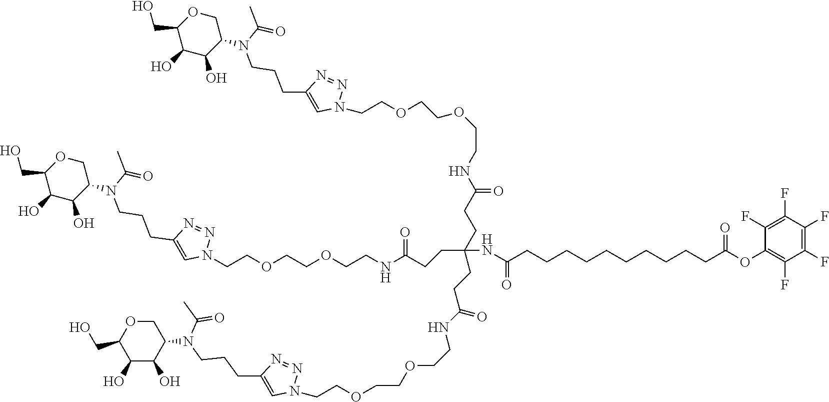 [SMILES: CC(=O)N(CCCc1cn(CCOCCOCCNC(=O)CCC(CCC(=O)NCCOCCOCCn2cc(CCCN(C(C)=O)[C@H]3CO[C@H](CO)[C@H](O)[C@@H]3O)nn2)(CCC(=O)NCCOCCOCCn2cc(CCCN(C(C)=O)[C@H]3CO[C@H](CO)[C@H](O)[C@@H]3O)nn2)NC(=O)CCCCCCCCCCC(=O)Oc2c(F)c(F)c(F)c(F)c2F)nn1)[C@H]1CO[C@H](CO)[C@H](O)[C@@H]1O] |
| 1908 | 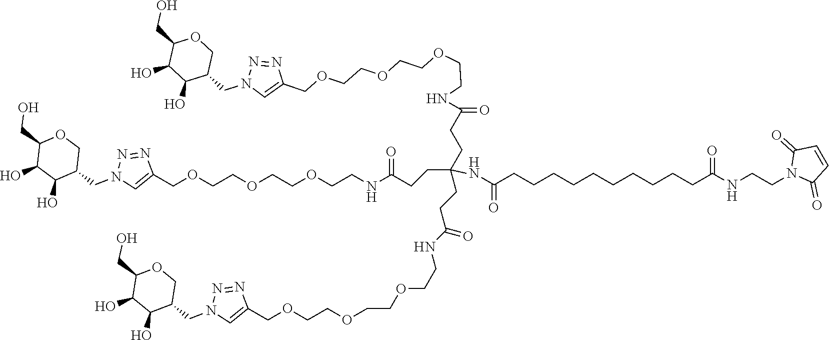 [SMILES: O=C(CCCCCCCCCCC(=O)NC(CCC(=O)NCCOCCOCCOCc1cn(C[C@H]2CO[C@H](CO)[C@H](O)[C@@H]2O)nn1)(CCC(=O)NCCOCCOCCOCc1cn(C[C@H]2CO[C@H](CO)[C@H](O)[C@@H]2O)nn1)CCC(=O)NCCOCCOCCOCc1cn(C[C@H]2CO[C@H](CO)[C@H](O)[C@@H]2O)nn1)NCCN1C(=O)C=CC1=O] |
| 1909 | 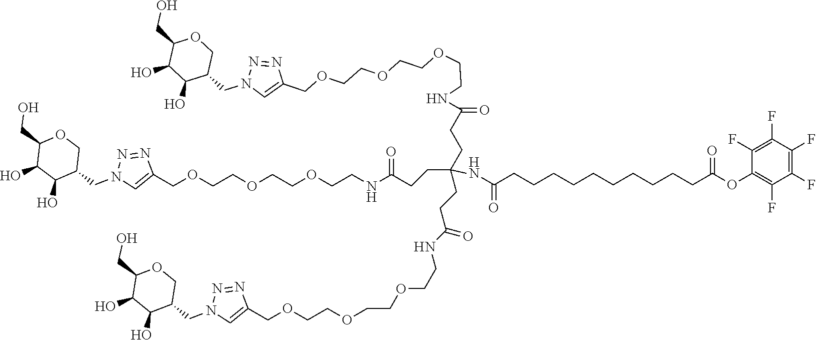 [SMILES: O=C(CCC(CCC(=O)NCCOCCOCCOCc1cn(C[C@H]2CO[C@H](CO)[C@H](O)[C@@H]2O)nn1)(CCC(=O)NCCOCCOCCOCc1cn(C[C@H]2CO[C@H](CO)[C@H](O)[C@@H]2O)nn1)NC(=O)CCCCCCCCCCC(=O)Oc1c(F)c(F)c(F)c(F)c1F)NCCOCCOCCOCc1cn(C[C@H]2CO[C@H](CO)[C@H](O)[C@@H]2O)nn1] |
| 1910 | 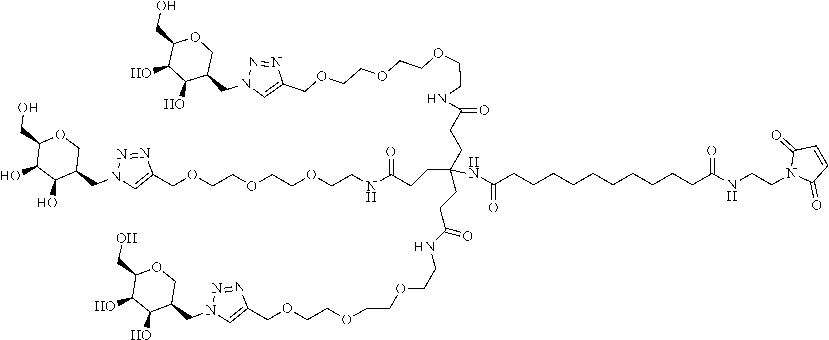 [SMILES: O=C(CCCCCCCCCCC(=O)NC(CCC(=O)NCCOCCOCCOCc1cn(C[C@@H]2CO[C@H](CO)[C@H](O)[C@@H]2O)nn1)(CCC(=O)NCCOCCOCCOCc1cn(C[C@@H]2CO[C@H](CO)[C@H](O)[C@@H]2O)nn1)CCC(=O)NCCOCCOCCOCc1cn(C[C@@H]2CO[C@H](CO)[C@H](O)[C@@H]2O)nn1)NCCN1C(=O)C=CC1=O] |
| 1911 | 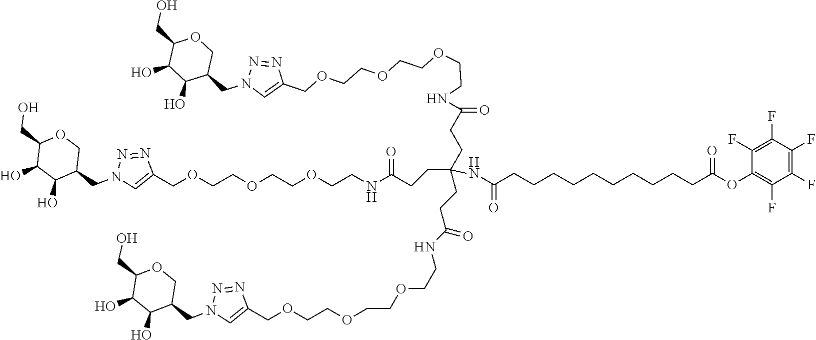 [SMILES: O=C(CCC(CCC(=O)NCCOCCOCCOCc1cn(C[C@@H]2CO[C@H](CO)[C@H](O)[C@@H]2O)nn1)(CCC(=O)NCCOCCOCCOCc1cn(C[C@@H]2CO[C@H](CO)[C@H](O)[C@@H]2O)nn1)NC(=O)CCCCCCCCCCC(=O)Oc1c(F)c(F)c(F)c(F)c1F)NCCOCCOCCOCc1cn(C[C@@H]2CO[C@H](CO)[C@H](O)[C@@H]2O)nn1] |
| 1912 | 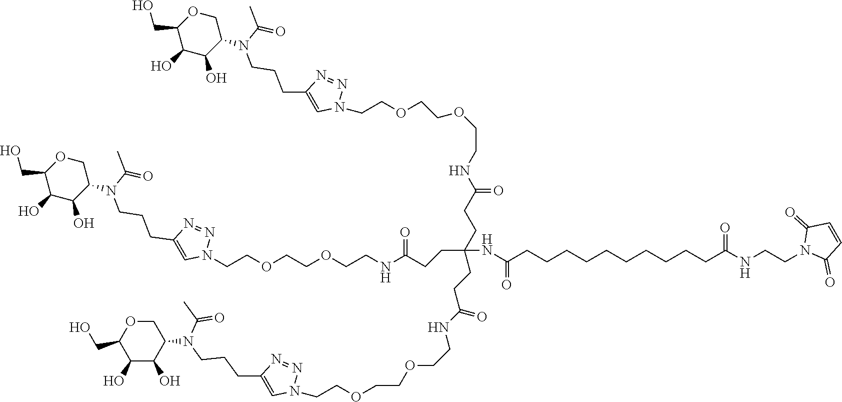 [SMILES: CC(=O)N(CCCc1cn(CCOCCOCCNC(=O)CCC(CCC(=O)NCCOCCOCCn2cc(CCCN(C(C)=O)[C@H]3CO[C@H](CO)[C@H](O)[C@@H]3O)nn2)(CCC(=O)NCCOCCOCCn2cc(CCCN(C(C)=O)[C@H]3CO[C@H](CO)[C@H](O)[C@@H]3O)nn2)NC(=O)CCCCCCCCCCC(=O)NCCN2C(=O)C=CC2=O)nn1)[C@H]1CO[C@H](CO)[C@H](O)[C@@H]1O] |
| 1913 | 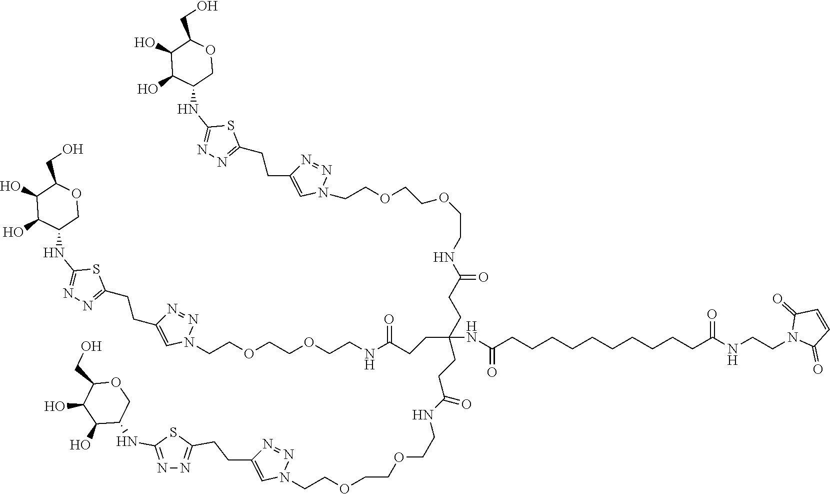 [SMILES: O=C(CCCCCCCCCCC(=O)NC(CCC(=O)NCCOCCOCCn1cc(CCc2nnc(N[C@H]3CO[C@H](CO)[C@H](O)[C@@H]3O)s2)nn1)(CCC(=O)NCCOCCOCCn1cc(CCc2nnc(N[C@H]3CO[C@H](CO)[C@H](O)[C@@H]3O)s2)nn1)CCC(=O)NCCOCCOCCn1cc(CCc2nnc(N[C@H]3CO[C@H](CO)[C@H](O)[C@@H]3O)s2)nn1)NCCN1C(=O)C=CC1=O] |
| 1914 | 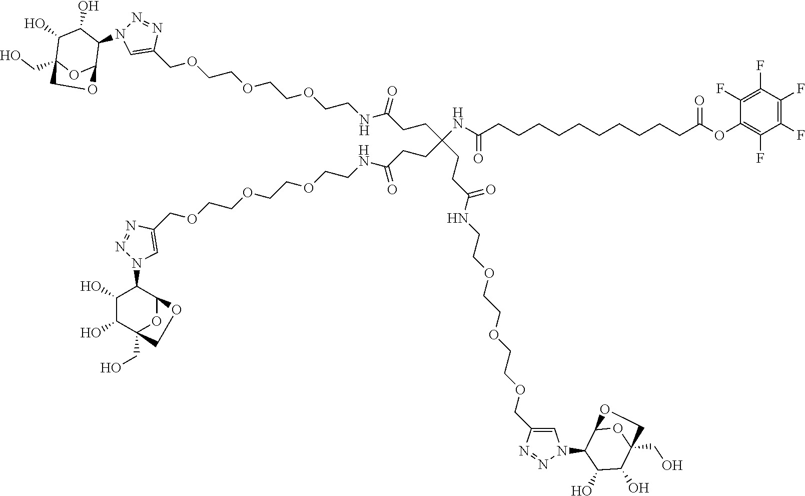 [SMILES: O=C(CCC(CCC(=O)NCCOCCOCCOCc1cn([C@H]2[C@H]3OC[C@](CO)(O3)[C@H](O)[C@@H]2O)nn1)(CCC(=O)NCCOCCOCCOCc1cn([C@H]2[C@H]3OC[C@](CO)(O3)[C@H](O)[C@@H]2O)nn1)NC(=O)CCCCCCCCCCC(=O)Oc1c(F)c(F)c(F)c(F)c1F)NCCOCCOCCOCc1cn([C@H]2[C@H]3OC[C@](CO)(O3)[C@H](O)[C@@H]2O)nn1] |
| 1915 | 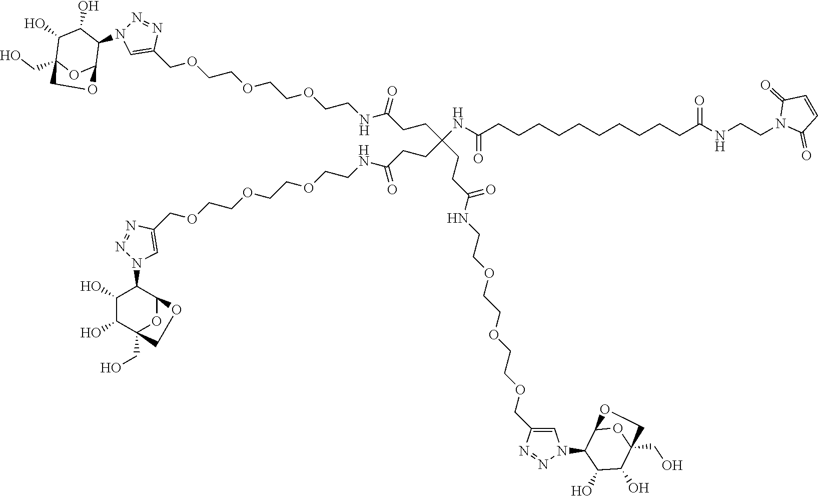 [SMILES: O=C(CCCCCCCCCCC(=O)NC(CCC(=O)NCCOCCOCCOCc1cn([C@H]2[C@H]3OC[C@](CO)(O3)[C@H](O)[C@@H]2O)nn1)(CCC(=O)NCCOCCOCCOCc1cn([C@H]2[C@H]3OC[C@](CO)(O3)[C@H](O)[C@@H]2O)nn1)CCC(=O)NCCOCCOCCOCc1cn([C@H]2[C@H]3OC[C@](CO)(O3)[C@H](O)[C@@H]2O)nn1)NCCN1C(=O)C=CC1=O] |
| 1915A | 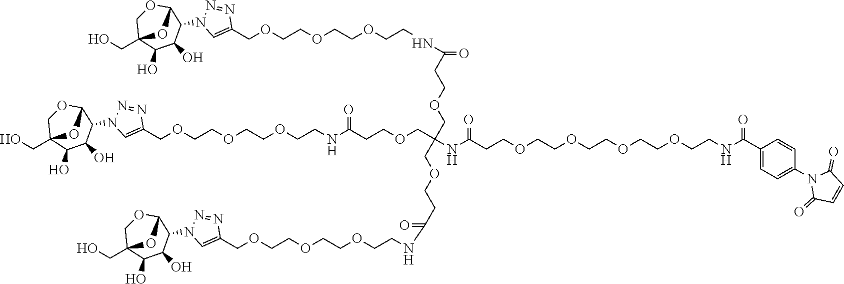 [SMILES: O=C(CCOCC(COCCC(=O)NCCOCCOCCOCc1cn([C@H]2[C@H]3OC[C@](CO)(O3)[C@H](O)[C@@H]2O)nn1)(COCCC(=O)NCCOCCOCCOCc1cn([C@H]2[C@H]3OC[C@](CO)(O3)[C@H](O)[C@@H]2O)nn1)NC(=O)CCOCCOCCOCCOCCNC(=O)c1ccc(N2C(=O)C=CC2=O)cc1)NCCOCCOCCOCc1cn([C@H]2[C@H]3OC[C@](CO)(O3)[C@H](O)[C@@H]2O)nn1] |
| 1916 | 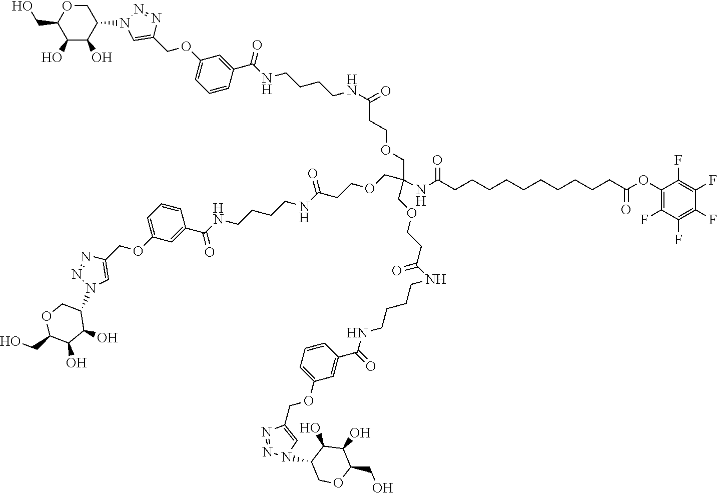 [SMILES: O=C(CCOCC(COCCC(=O)NCCCCNC(=O)c1cccc(OCc2cn([C@H]3CO[C@H](CO)[C@H](O)[C@@H]3O)nn2)c1)(COCCC(=O)NCCCCNC(=O)c1cccc(OCc2cn([C@H]3CO[C@H](CO)[C@H](O)[C@@H]3O)nn2)c1)NC(=O)CCCCCCCCCCC(=O)Oc1c(F)c(F)c(F)c(F)c1F)NCCCCNC(=O)c1cccc(OCc2cn([C@H]3CO[C@H](CO)[C@H](O)[C@@H]3O)nn2)c1] |
| 1917 | 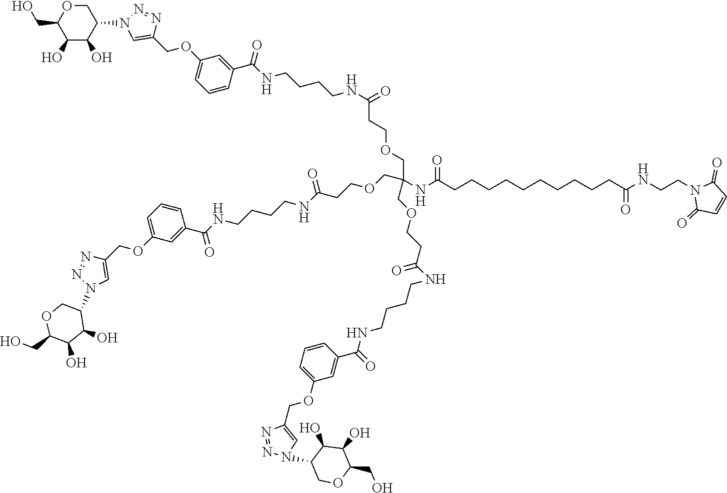 [SMILES: O=C(CCCCCCCCCCC(=O)NC(COCCC(=O)NCCCCNC(=O)c1cccc(OCc2cn([C@H]3CO[C@H](CO)[C@H](O)[C@@H]3O)nn2)c1)(COCCC(=O)NCCCCNC(=O)c1cccc(OCc2cn([C@H]3CO[C@H](CO)[C@H](O)[C@@H]3O)nn2)c1)COCCC(=O)NCCCCNC(=O)c1cccc(OCc2cn([C@H]3CO[C@H](CO)[C@H](O)[C@@H]3O)nn2)c1)NCCN1C(=O)C=CC1=O] |
| 1918 (I- 165) | 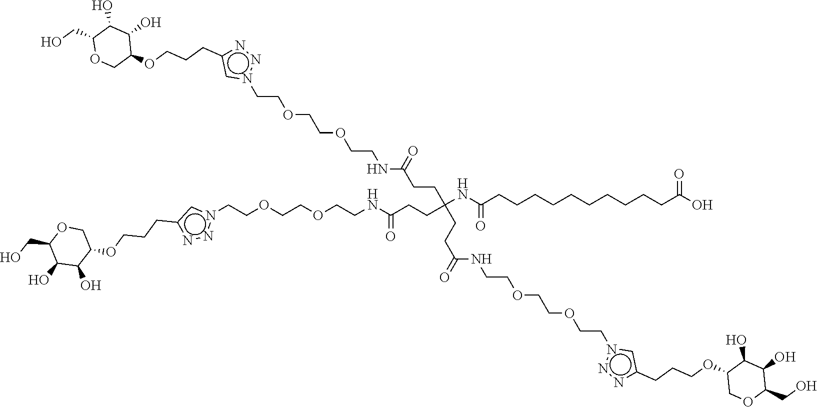 |
| 1919 (I- 166) | 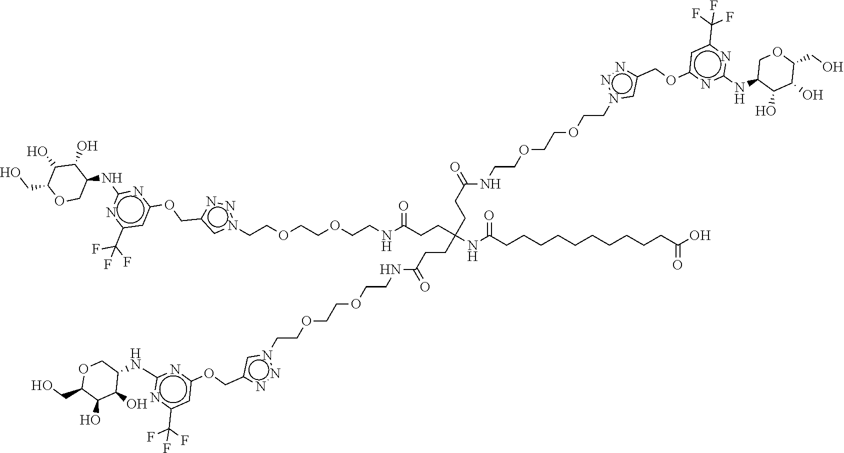 |
| 1920 (I- 167) | 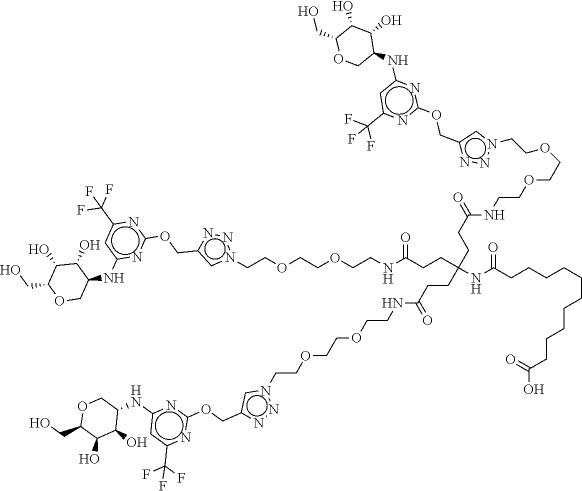 |
| 1921 | 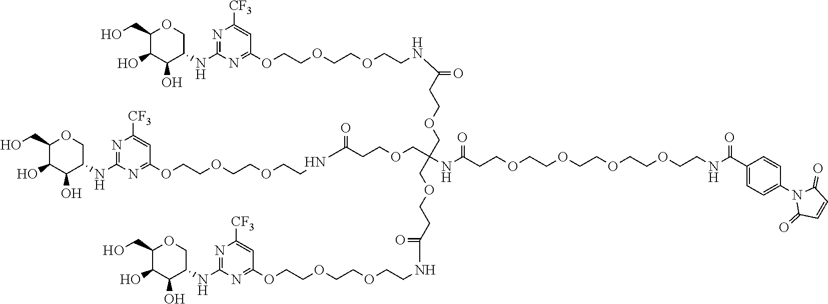 [SMILES: O=C(CCOCC(COCCC(=O)NCCOCCOCCOc1cc(C(F)(F)F)nc(N[C@H]2CO[C@H](CO)[C@H](O)[C@@H]2O)n1)(COCCC(=O)NCCOCCOCCOc1cc(C(F)(F)F)nc(N[C@H]2CO[C@H](CO)[C@H](O)[C@@H]2O)n1)NC(=O)CCOCCOCCOCCOCCNC(=O)c1ccc(N2C(=O)C=CC2=O)cc1)NCCOCCOCCOc1cc(C(F)(F)F)nc(N[C@H]2CO[C@H](CO)[C@H](O)[C@@H]2O)n1] |
| 1921A | 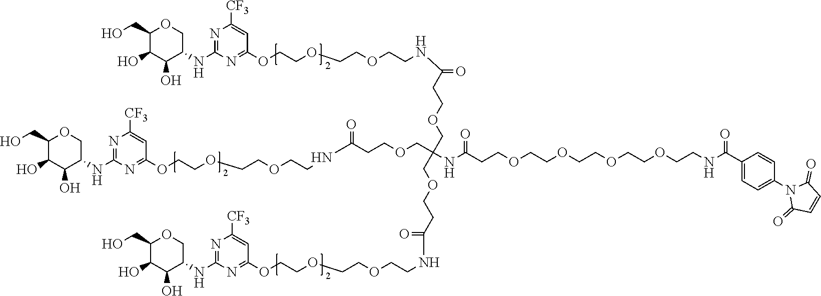 |
| 1922 | 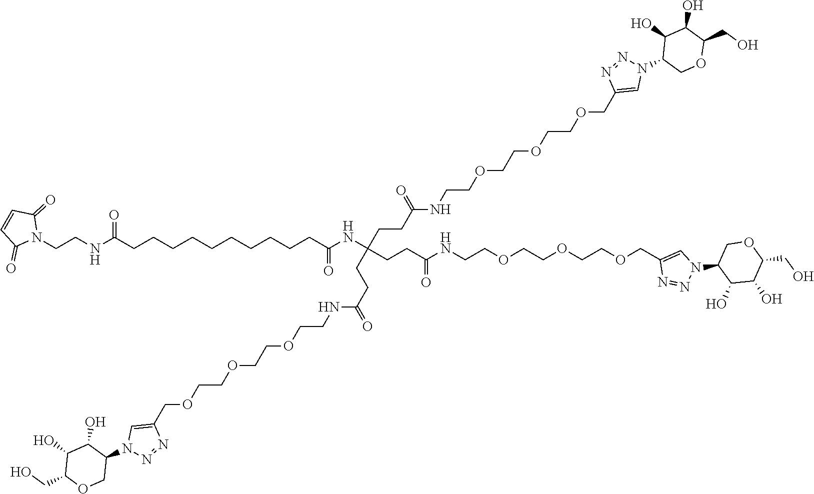 [SMILES: O=C(CCCCCCCCCCC(=O)NC(CCC(=O)NCCOCCOCCOCc1cn([C@H]2CO[C@H](CO)[C@H](O)[C@@H]2O)nn1)(CCC(=O)NCCOCCOCCOCc1cn([C@H]2CO[C@H](CO)[C@H](O)[C@@H]2O)nn1)CCC(=O)NCCOCCOCCOCc1cn([C@H]2CO[C@H](CO)[C@H](O)[C@@H]2O)nn1)NCCN1C(=O)C=CC1=O] |
| 1923 | 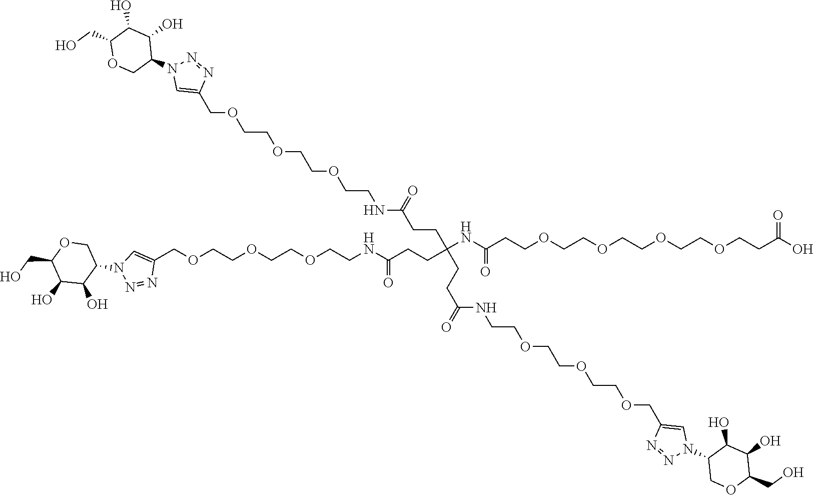 [SMILES: O=C(O)CCOCCOCCOCCOCCC(=O)NC(CCC(=O)NCCOCCOCCOCc1cn([C@H]2CO[C@H](CO)[C@H](O)[C@@H]2O)nn1)(CCC(=O)NCCOCCOCCOCc1cn([C@H]2CO[C@H](CO)[C@H](O)[C@@H]2O)nn1)CCC(=O)NCCOCCOCCOCc1cn([C@H]2CO[C@H](CO)[C@H](O)[C@@H]2O)nn1] |
| 1924 | 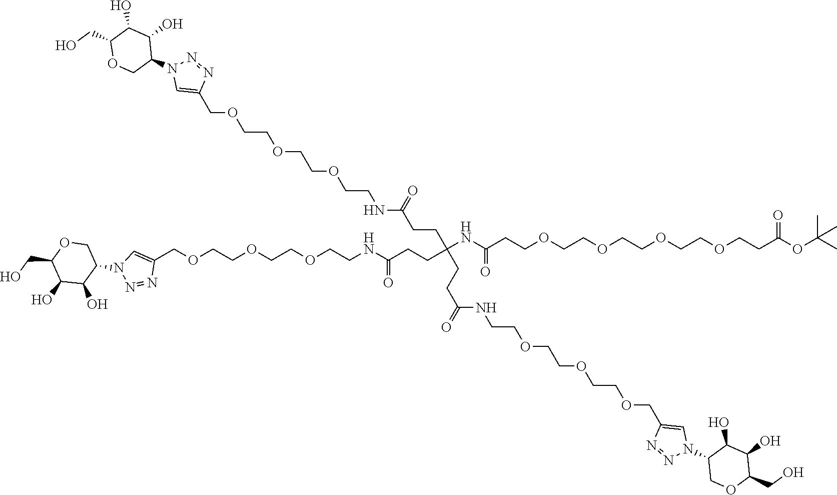 [SMILES: CC(C)(C)OC(=O)CCOCCOCCOCCOCCC(=O)NC(CCC(=O)NCCOCCOCCOCc1cn([C@H]2CO[C@H](CO)[C@H](O)[C@@H]2O)nn1)(CCC(=O)NCCOCCOCCOCc1cn([C@H]2CO[C@H](CO)[C@H](O)[C@@H]2O)nn1)CCC(=O)NCCOCCOCCOCc1cn([C@H]2CO[C@H](CO)[C@H](O)[C@@H]2O)nn1] |
| 1925 | 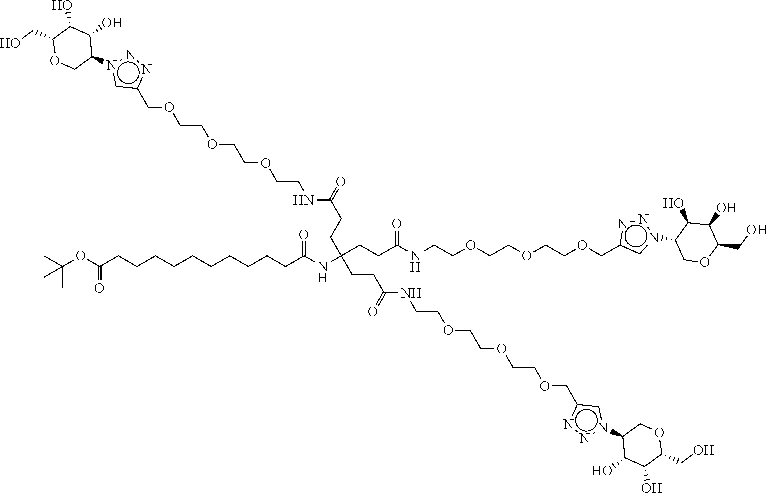 |
| 1926 | 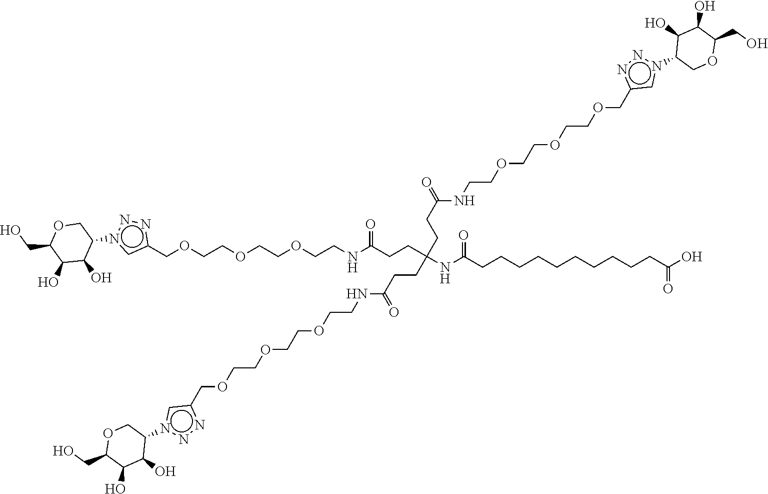 |

Table 20 illustrates exemplary monovalent ASGPR binding intermediate compounds of this disclosure that include a promoiety and X groups that are of formula (Ib).

TABLE 20
Multivalent ASGPR binding intermediate compounds of formula (Ib)

| # | Structure |
|---|---|
| 2001 (I-173) | 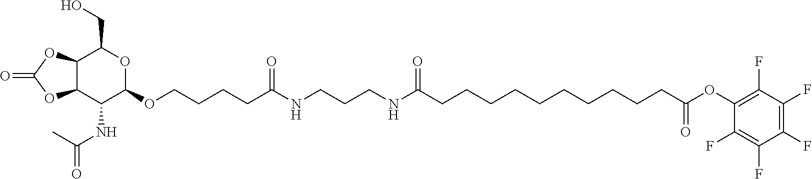 [SMILES: CC(=O)N[C@H]1[C@H](OCCCCC(=O)NCCCNC(=O)CCCCCCCCCCC(=O)Oc2c(F)c(F)c(F)c(F)c2F)O[C@H](CO)[C@@H]2OC(=O)O[C@H]12] |

Table 21 illustrates exemplary ASGPR binding intermediate compounds of this disclosure that include X groups that are of formula (In).

TABLE 21
ASGPR binding intermediate compounds including X groups of formula (In)

| # | Structure |
|---|---|
| 2101 | 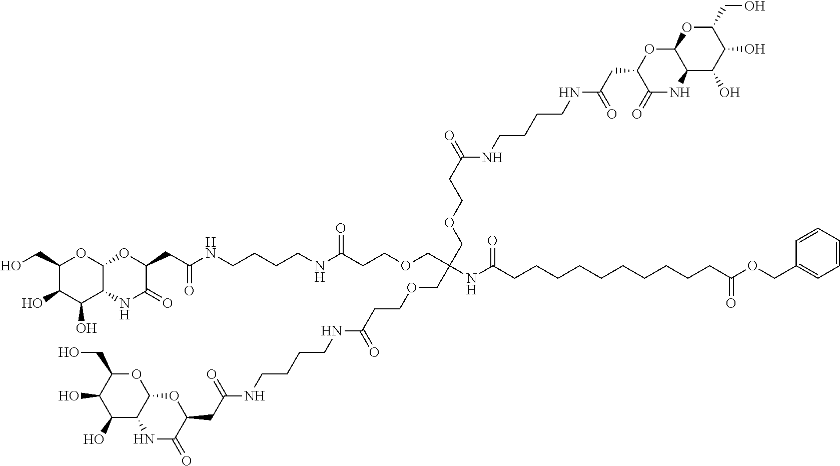 [SMILES: O=C(CCOCC(COCCC(=O)NCCCCNC(=O)C[C@@H]1O[C@H]2O[C@H](CO)[C@H](O)[C@H](O)[C@H]2NC1=O)(COCCC(=O)NCCCCNC(=O)C[C@@H]1O[C@H]2O[C@H](CO)[C@H](O)[C@H](O)[C@H]2NC1=O)NC(=O)CCCCCCCCCCC(=O)OCc1ccccc1)NCCCCNC(=O)C[C@@H]1O[C@H]2O[C@H](CO)[C@H](O)[C@H](O)[C@H]2NC1=O] |

Table 21A illustrates exemplary ASGPR binding intermediate compounds.

TABLE 21A
ASGPR binding intermediate compounds

| # | Structure |
|---|---|
| 2301 | 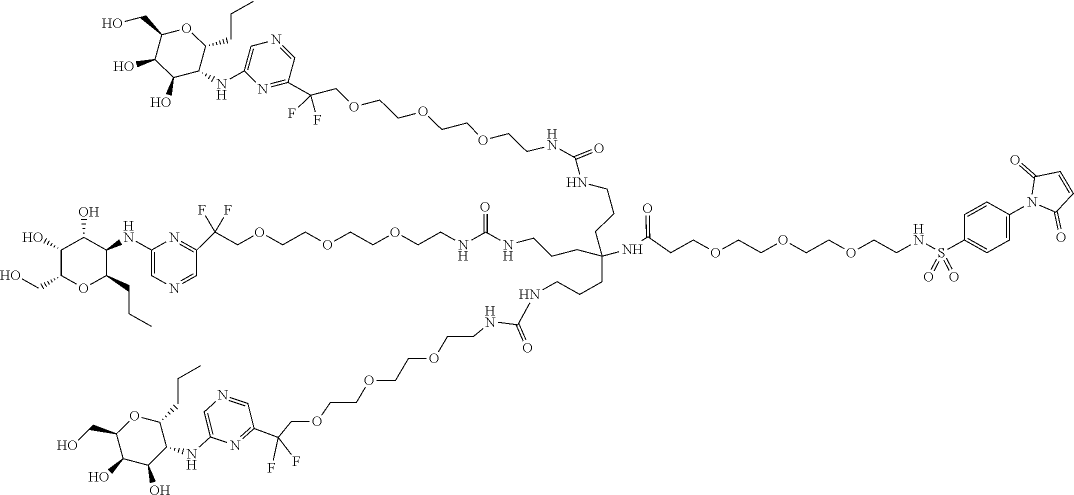 [SMILES: CCC[C@H]1O[C@H](CO)[C@H](O)[C@H](O)[C@H]1Nc1cncc(C(F)(F)COCCOCCOCCNC(=O)NCCCC(CCCNC(=O)NCCOCCOCCOCC(F)(F)c2cncc(N[C@@H]3[C@@H](O)[C@@H](O)[C@@H](CO)O[C@@H]3CCC)n2)(CCCNC(=O)NCCOCCOCCOCC(F)(F)c2cncc(N[C@@H]3[C@@H](O)[C@@H](O)[C@@H](CO)O[C@@H]3CCC)n2)NC(=O)CCOCCOCCOCCNS(=O)(=O)c2ccc(N3C(=O)C=CC3=O)cc2)n1] |
| 2302 | 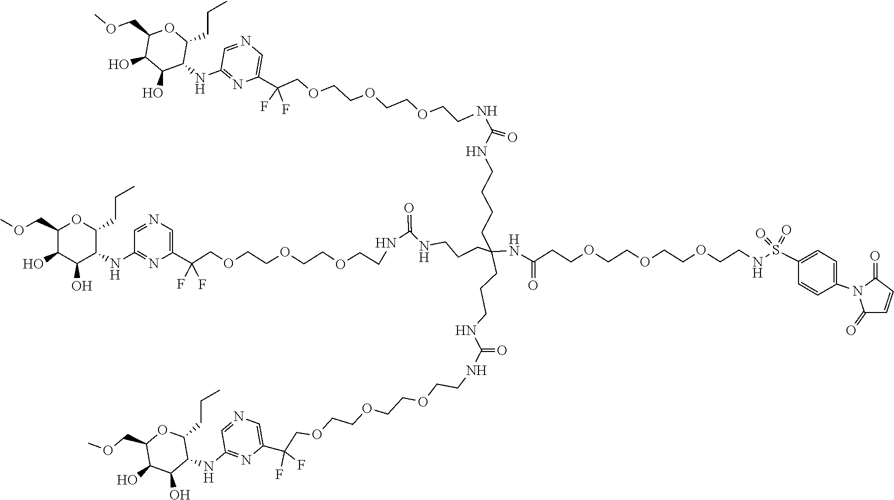 [SMILES: CCC[C@H]1O[C@H](COC)[C@H](O)[C@H](O)[C@H]1Nc1cncc(C(F)(F)COCCOCCOCCNC(=O)NCCCCC(CCCNC(=O)NCCOCCOCCOCC(F)(F)c2cncc(N[C@@H]3[C@@H](O)[C@@H](O)[C@@H](COC)O[C@@H]3CCC)n2)(CCCNC(=O)NCCOCCOCCOCC(F)(F)c2cncc(N[C@@H]3[C@@H](O)[C@@H](O)[C@@H](COC)O[C@@H]3CCC)n2)NC(=O)CCOCCOCCOCCNS(=O)(=O)c2ccc(N3C(=O)C=CC3=O)cc2)n1] |
| 2303 | 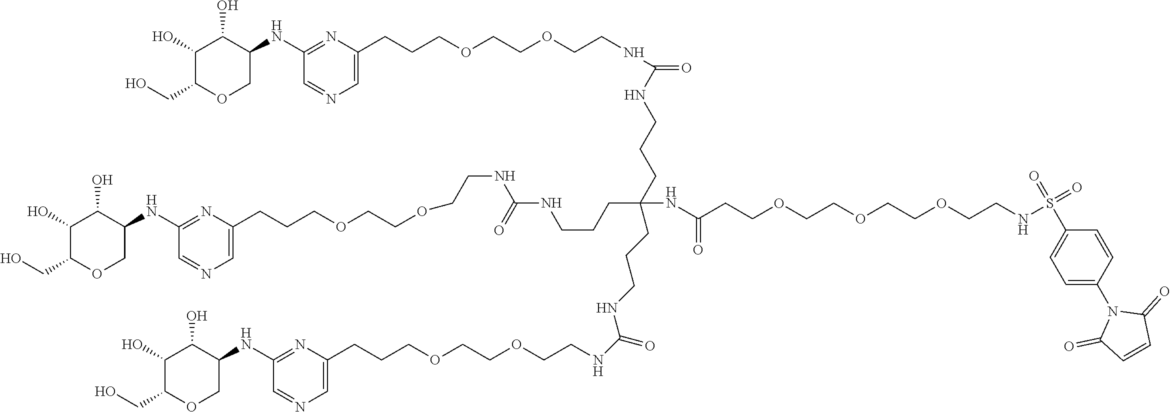 [SMILES: O=C(CCOCCOCCOCCNS(=O)(=O)c1ccc(N2C(=O)C=CC2=O)cc1)NC(CCCNC(=O)NCCOCCOCCCc1cncc(N[C@H]2CO[C@H](CO)[C@H](O)[C@@H]2O)n1)(CCCNC(=O)NCCOCCOCCCc1cncc(N[C@H]2CO[C@H](CO)[C@H](O)[C@@H]2O)n1)CCCNC(=O)NCCOCCOCCCc1cncc(N[C@H]2CO[C@H](CO)[C@H](O)[C@@H]2O)n1] |
| 2304 | 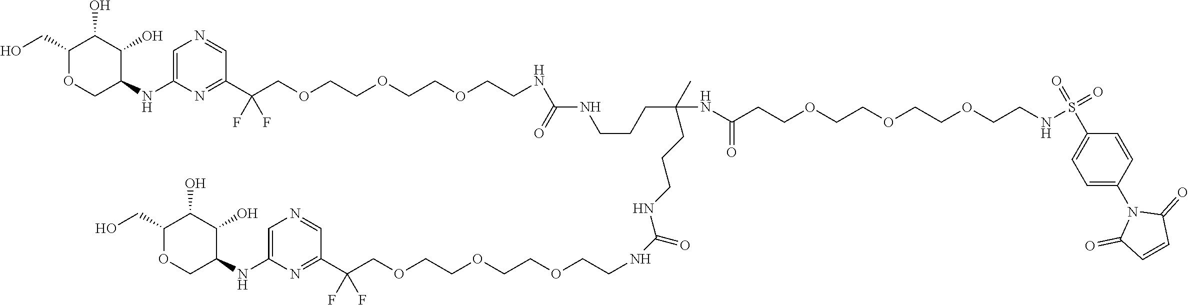 [SMILES: CC(CCCNC(=O)NCCOCCOCCOCC(F)(F)c1cncc(N[C@H]2CO[C@H](CO)[C@H](O)[C@@H]2O)n1)(CCCNC(=O)NCCOCCOCCOCC(F)(F)c1cncc(N[C@H]2CO[C@H](CO)[C@H](O)[C@@H]2O)n1)NC(=O)CCOCCOCCOCCNS(=O)(=O)c1ccc(N2C(=O)C=CC2=O)cc1] |
| 2305 | 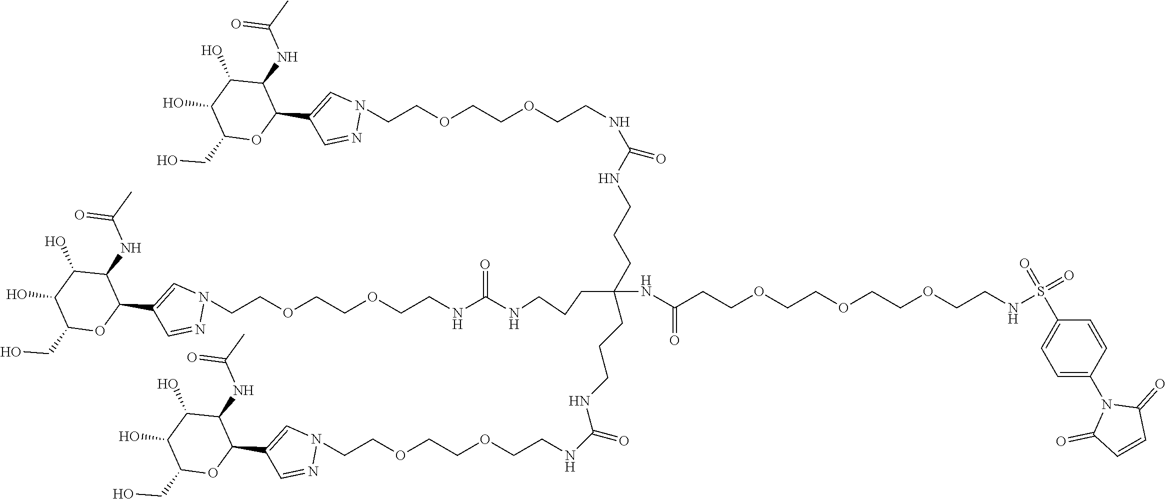 [SMILES: CC(=O)N[C@@H]1[C@@H](O)[C@@H](O)[C@@H](CO)O[C@@H]1c1cnn(CCOCCOCCNC(=O)NCCCC(CCCNC(=O)NCCOCCOCCn2cc([C@H]3O[C@H](CO)[C@H](O)[C@H](O)[C@H]3NC(C)=O)cn2)(CCCNC(=O)NCCOCCOCCn2cc([C@H]3O[C@H](CO)[C@H](O)[C@H](O)[C@H]3NC(C)=O)cn2)NC(=O)CCOCCOCCOCCNS(=O)(=O)c2ccc(N3C(=O)C=CC3=O)cc2)c1] |
| 2306 | 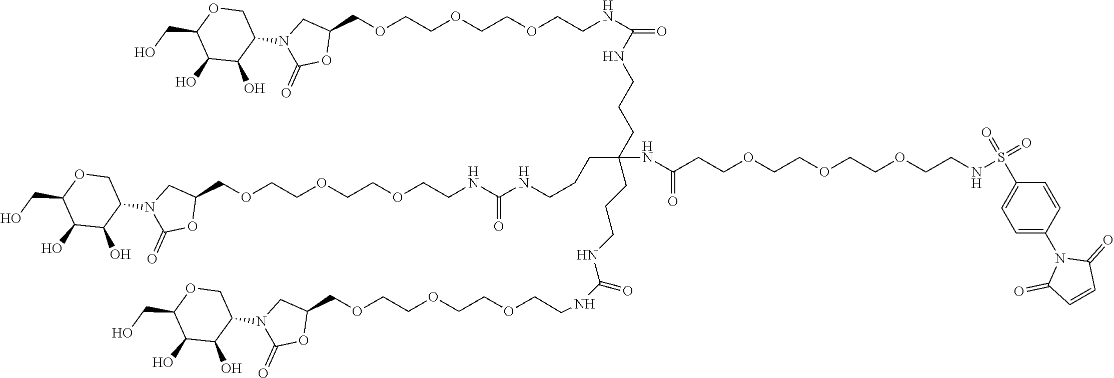 [SMILES: O=C(CCOCCOCCOCCNS(=O)(=O)c1ccc(N2C(=O)C=CC2=O)cc1)NC(CCCNC(=O)NCCOCCOCCOC[C@@H]1CN([C@H]2CO[C@H](CO)[C@H](O)[C@@H]2O)C(=O)O1)(CCCNC(=O)NCCOCCOCCOC[C@@H]1CN([C@H]2CO[C@H](CO)[C@H](O)[C@@H]2O)C(=O)O1)CCCNC(=O)NCCOCCOCCOC[C@@H]1CN([C@H]2CO[C@H](CO)[C@H](O)[C@@H]2O)C(=O)O1] |
| 2307 | 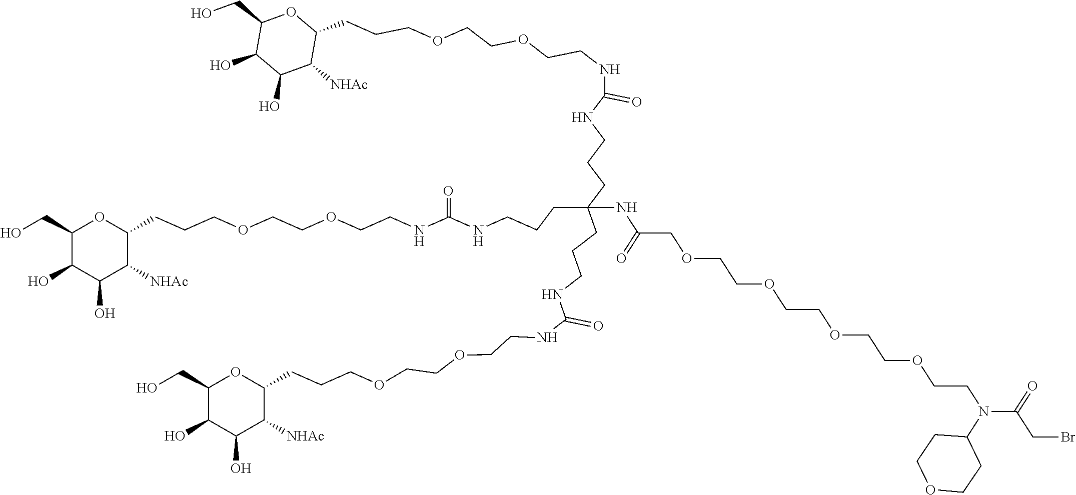 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)COCCOCCOCCOCCN(C(=O)CBr)C1CCOCC1] |
| 2308 | 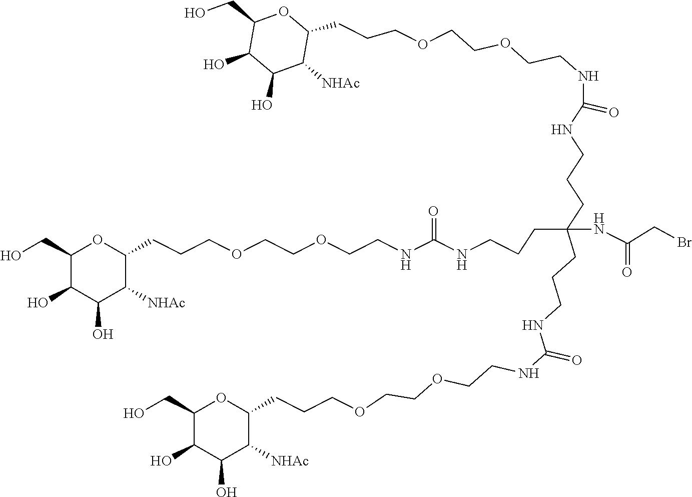 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)CBr] |
| 2309 | 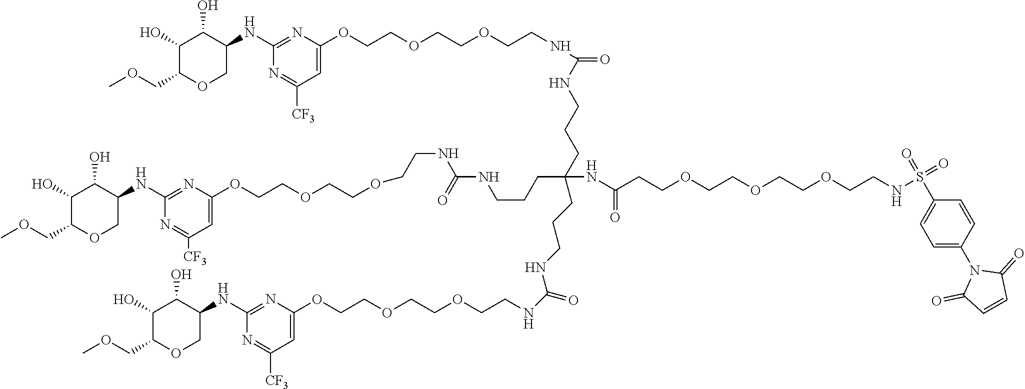 [SMILES: COC[C@H]1OC[C@H](Nc2nc(OCCOCCOCCNC(=O)NCCCC(CCCNC(=O)NCCOCCOCCOc3cc(C(F)(F)F)nc(N[C@H]4CO[C@H](COC)[C@H](O)[C@@H]4O)n3)(CCCNC(=O)NCCOCCOCCOc3cc(C(F)(F)F)nc(N[C@H]4CO[C@H](COC)[C@H](O)[C@@H]4O)n3)NC(=O)CCOCCOCCOCCNS(=O)(=O)c3ccc(N4C(=O)C=CC4=O)cc3)cc(C(F)(F)F)n2)[C@@H](O)[C@H]1O] |
| 2310 | 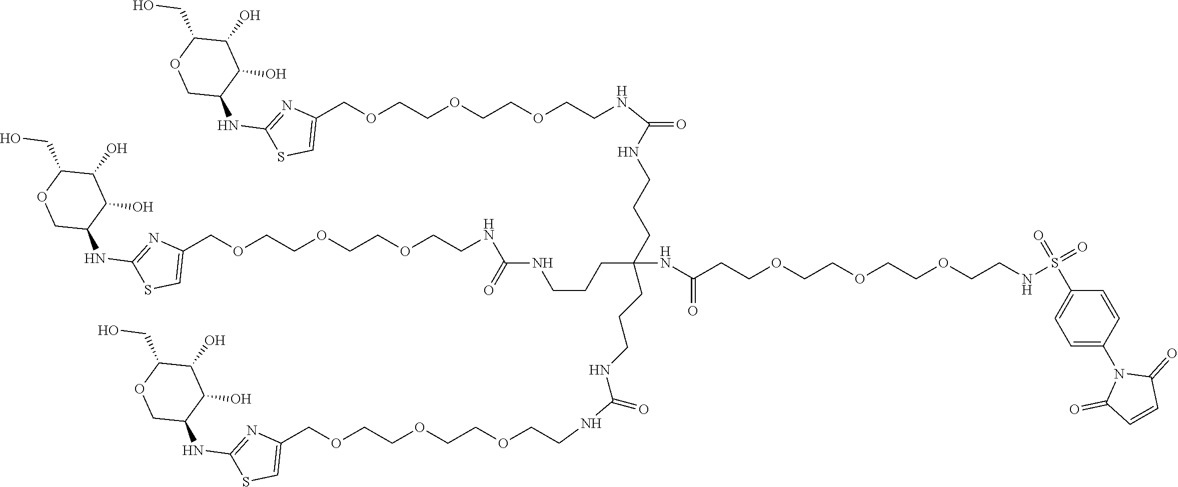 [SMILES: O=C(CCOCCOCCOCCNS(=O)(=O)c1ccc(N2C(=O)C=CC2=O)cc1)NC(CCCNC(=O)NCCOCCOCCOCc1csc(N[C@H]2CO[C@H](CO)[C@H](O)[C@@H]2O)n1)(CCCNC(=O)NCCOCCOCCOCc1csc(N[C@H]2CO[C@H](CO)[C@H](O)[C@@H]2O)n1)CCCNC(=O)NCCOCCOCCOCc1csc(N[C@H]2CO[C@H](CO)[C@H](O)[C@@H]2O)n1] |
| 2311 | 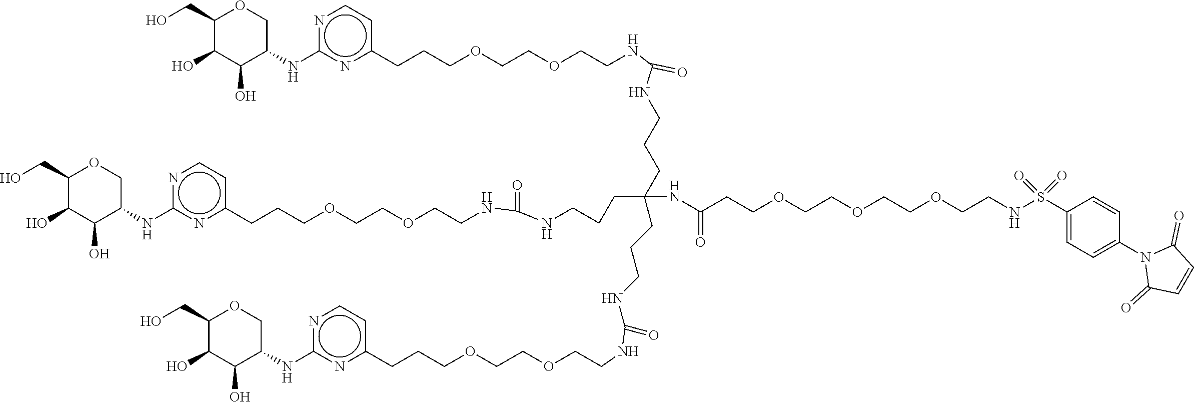 |
| 2312 | 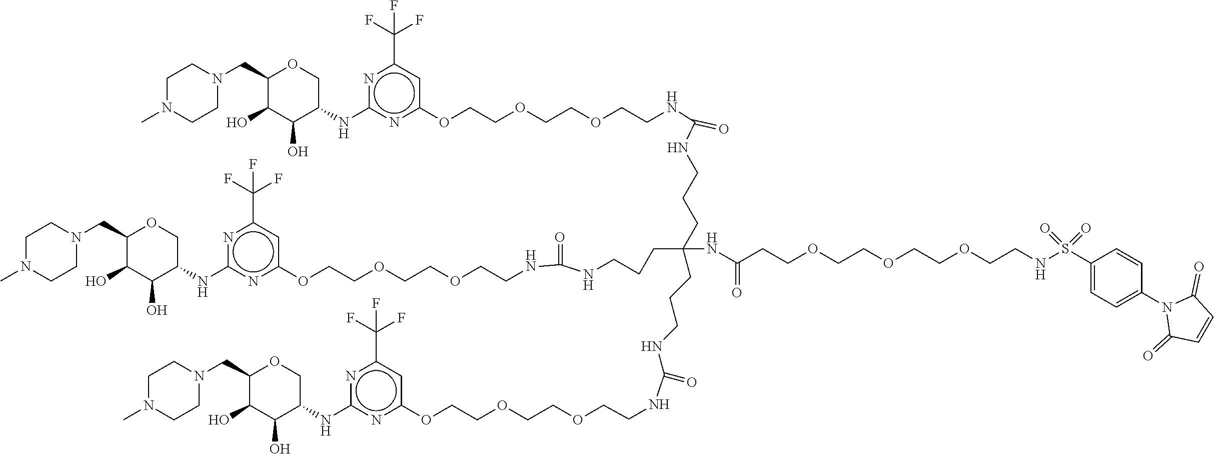 |
| 2313 | 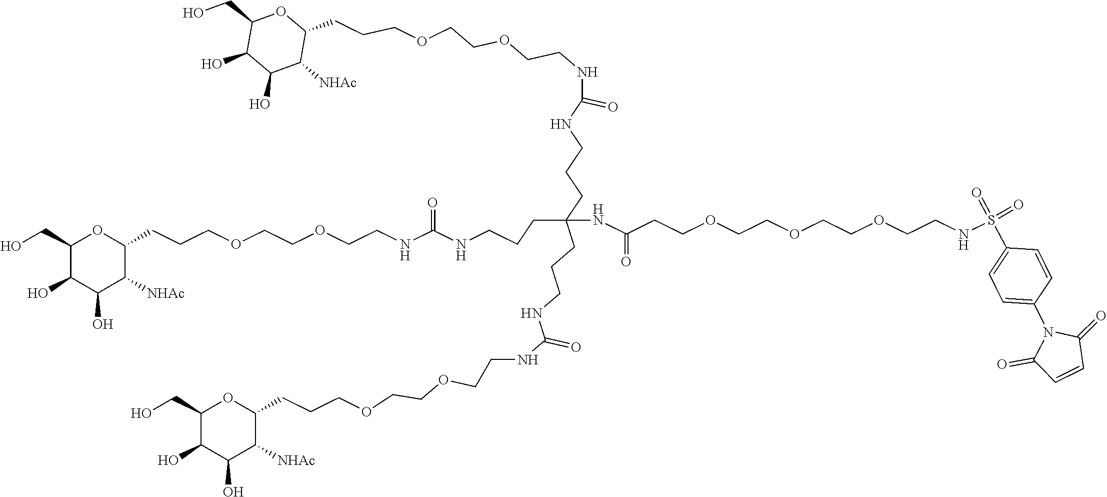 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)CCOCCOCCOCCNS(=O)(=O)c1ccc(N2C(=O)C=CC2=O)cc1] |
| 2314 | 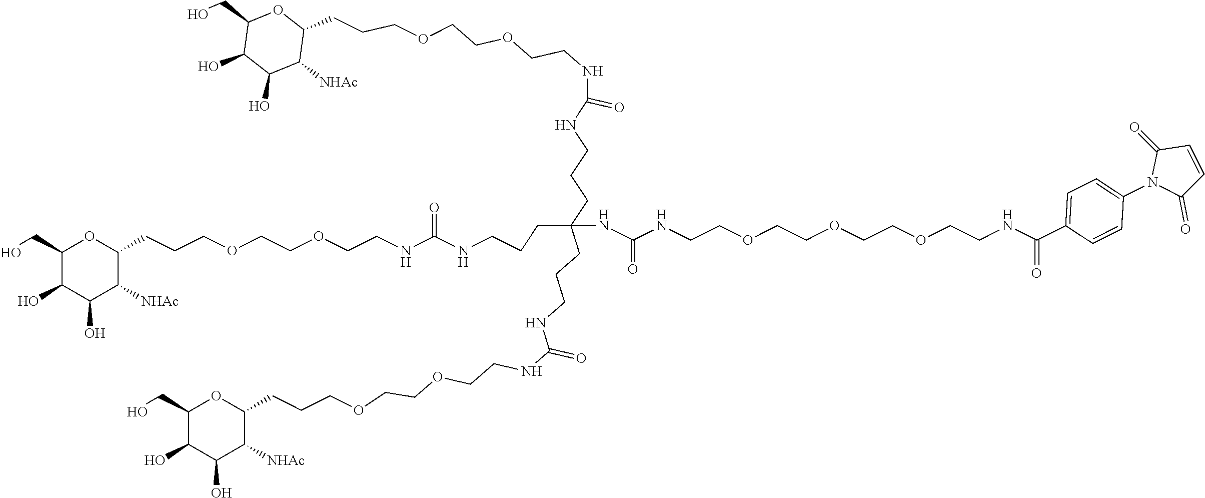 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2315 | 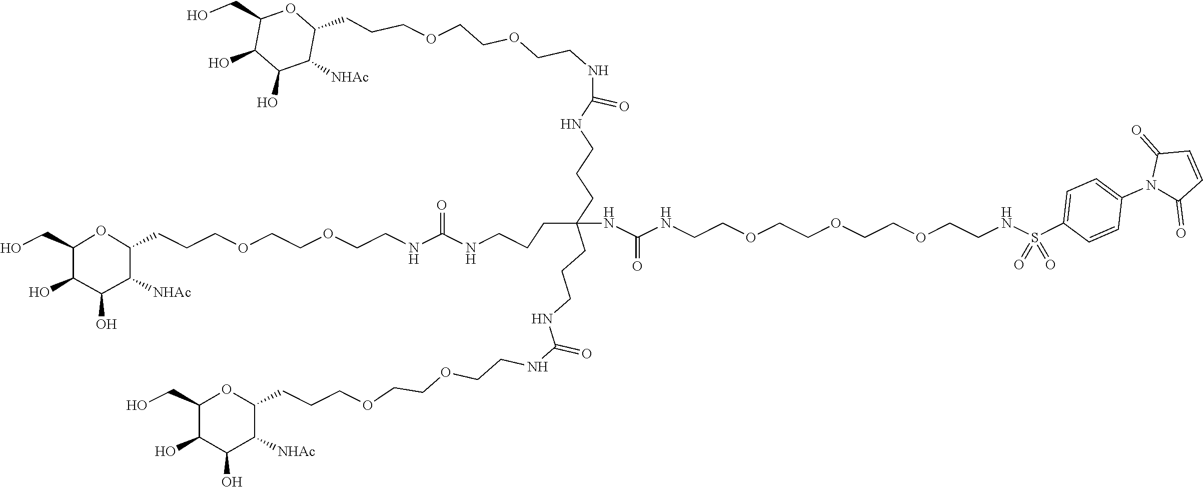 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCNS(=O)(=O)c1ccc(N2C(=O)C=CC2=O)cc1] |
| 2316 | 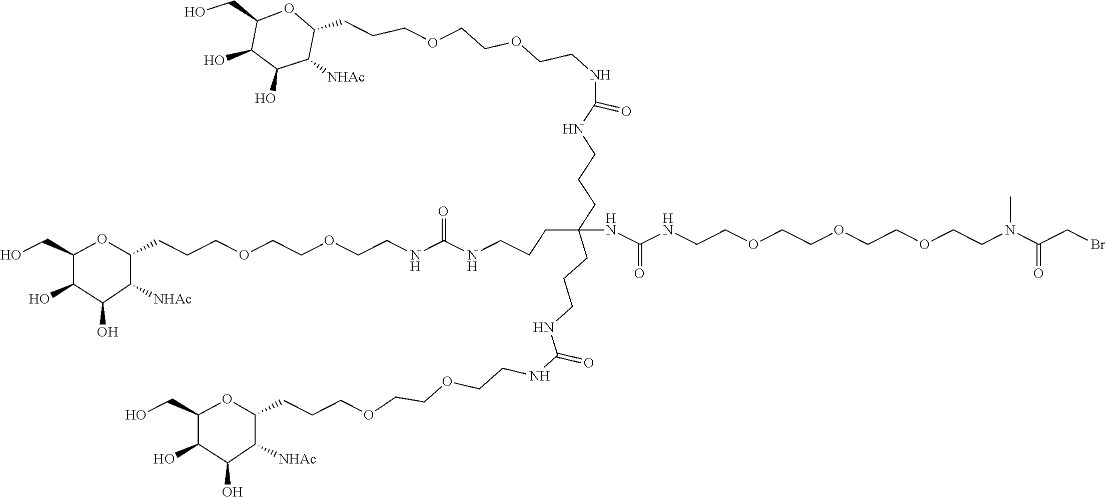 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCN(C)C(=O)CBr] |
| 2317 | 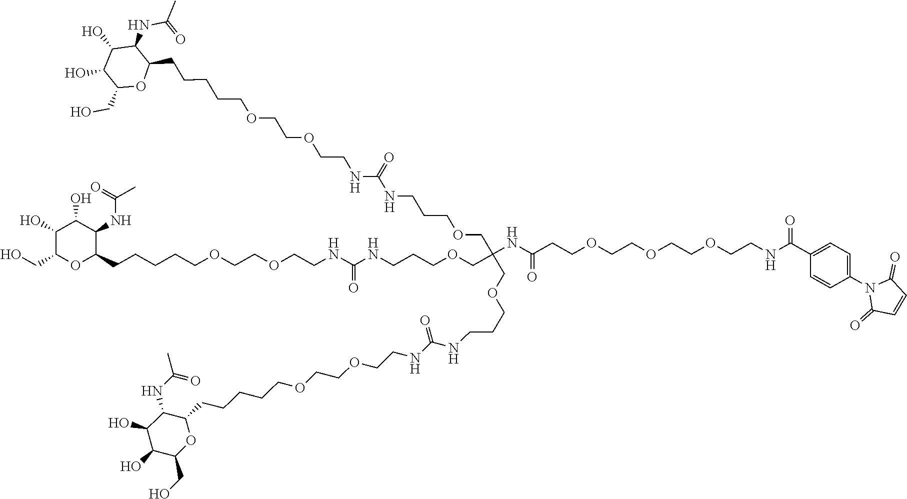 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@@H]1O[C@@H](CO)[C@@H](O)[C@@H](O)[C@@H]1NC(C)=O)(COCCCNC(=O)NCCOCCOCCCCC[C@H]1O[C@H](CO)[C@H](O)[C@H](O)[C@H]1NC(C)=O)NC(=O)CCOCCOCCOCCNC(=O)c1ccc(N2C(=O)C=CC2=O)cc1] |
| 2318 | 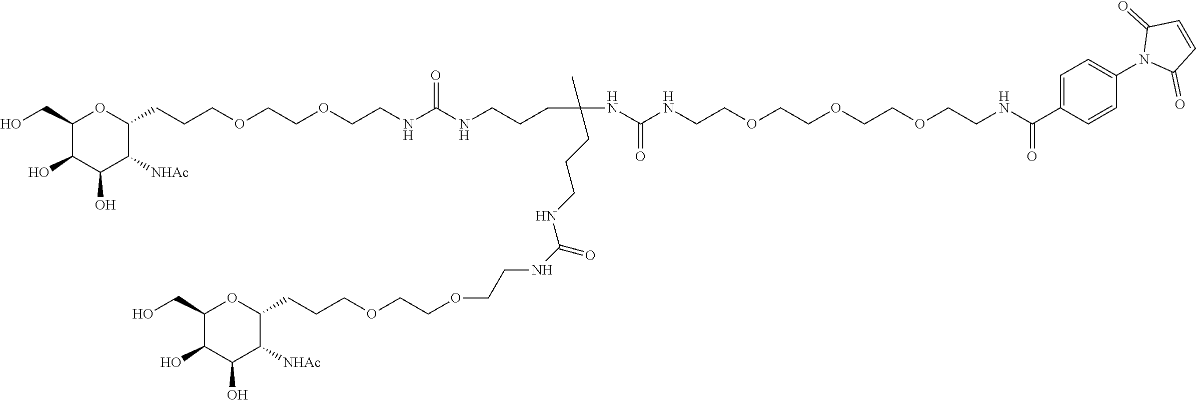 [SMILES: CC(=O)N[C@@H]1[C@@H](O)[C@@H](O)[C@@H](CO)O[C@@H]1CCCOCCOCCNC(=O)NCCCC(C)(CCCNC(=O)NCCOCCOCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2319- C | 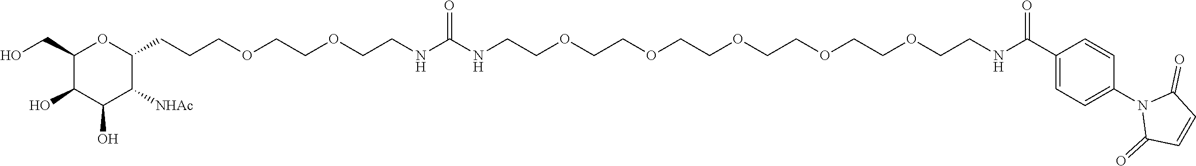 [SMILES: CC(=O)N[C@@H]1[C@@H](O)[C@@H](O)[C@@H](CO)O[C@@H]1CCCOCCOCCNC(=O)NCCOCCOCCOCCOCCOCCNC(=O)c1ccc(N2C(=O)C=CC2=O)cc1] |
| 2320 | 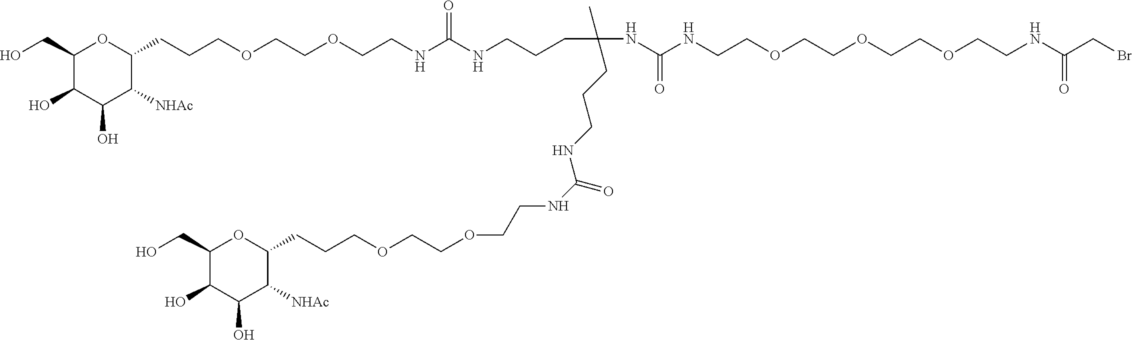 [SMILES: CC(=O)N[C@@H]1[C@@H](O)[C@@H](O)[C@@H](CO)O[C@@H]1CCCOCCOCCNC(=O)NCCCC(C)(CCCNC(=O)NCCOCCOCCC[C@H]1O[C@H](CO)[C@H](O)[C@H](O)[C@H]1NC(C)=O)NC(=O)NCCOCCOCCOCCNC(=O)CBr] |
| 2321 | 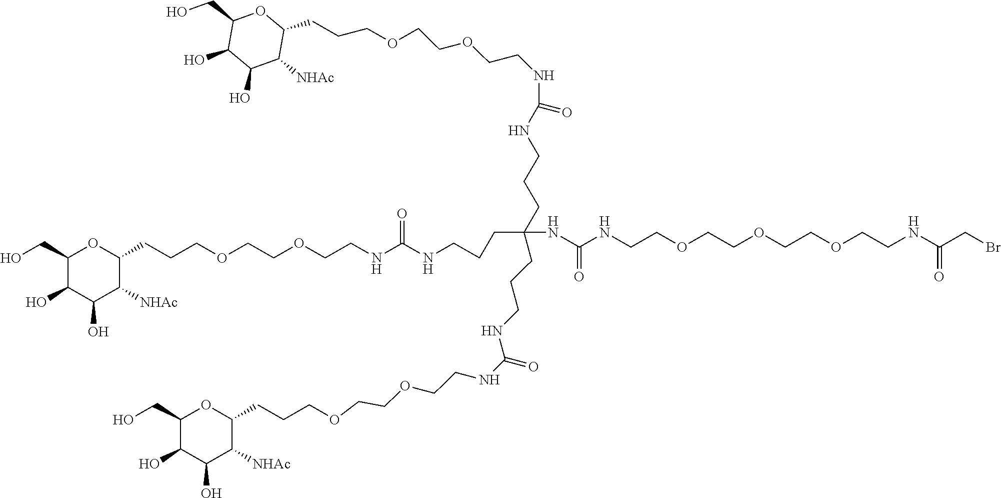 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCNC(=O)CBr] |
| 2322 | 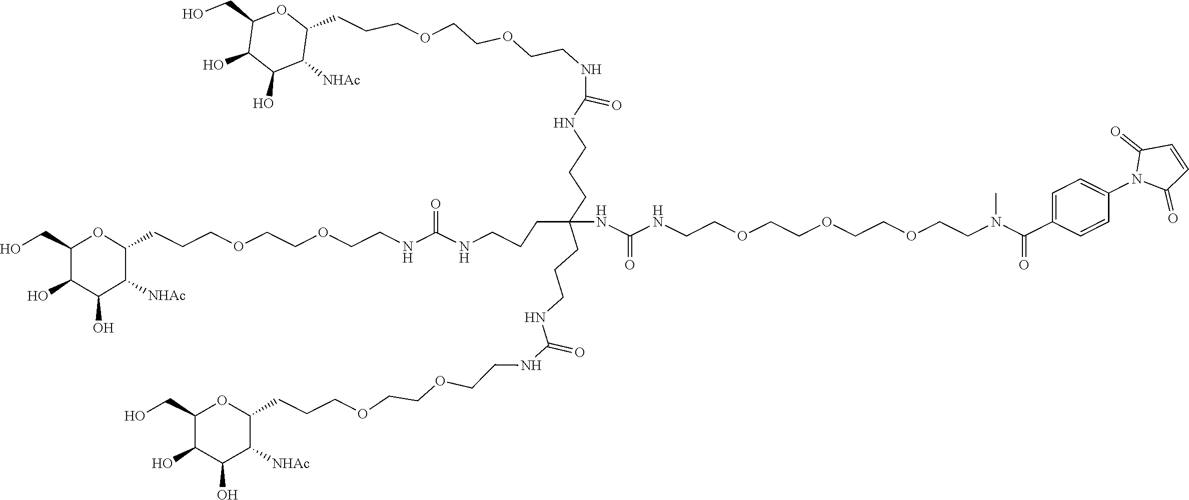 [SMILES: CC(=O)N[C@@H]1[C@@H](O)[C@@H](O)[C@@H](CO)O[C@@H]1CCCOCCOCCNC(=O)NCCCC(CCCNC(=O)NCCOCCOCCC[C@H]1O[C@H](CO)[C@H](O)[C@H](O)[C@H]1NC(C)=O)(CCCNC(=O)NCCOCCOCCC[C@H]1O[C@H](CO)[C@H](O)[C@H](O)[C@H]1NC(C)=O)NC(=O)NCCOCCOCCOCCN(C)C(=O)c1ccc(N2C(=O)C=CC2=O)cc1] |
| 2323 | 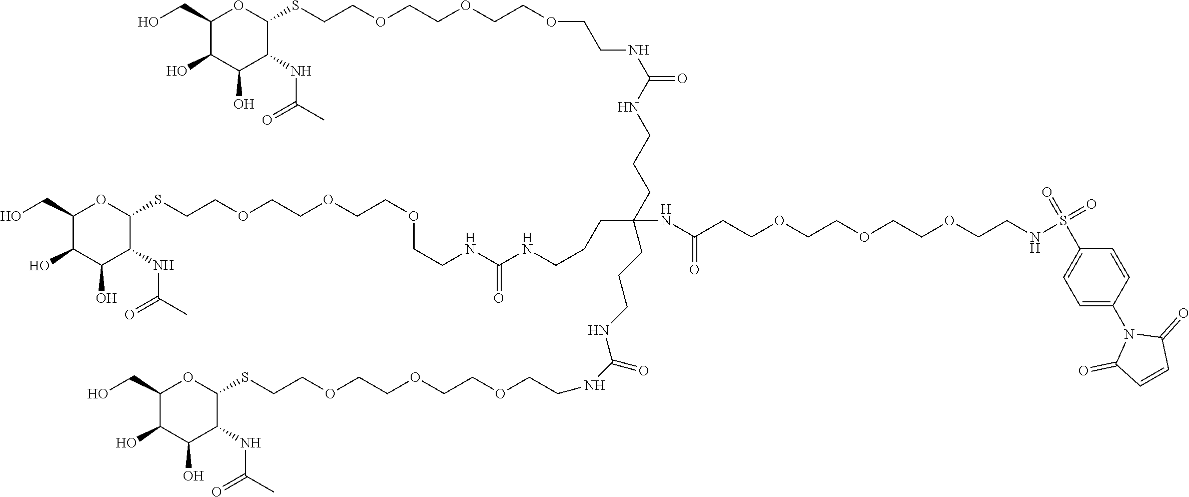 [SMILES: CC(=O)N[C@@H]1[C@@H](O)[C@@H](O)[C@@H](CO)O[C@@H]1SCCOCCOCCOCCNC(=O)NCCCC(CCCNC(=O)NCCOCCOCCOCCS[C@H]1O[C@H](CO)[C@H](O)[C@H](O)[C@H]1NC(C)=O)(CCCNC(=O)NCCOCCOCCOCCS[C@H]1O[C@H](CO)[C@H](O)[C@H](O)[C@H]1NC(C)=O)NC(=O)CCOCCOCCOCCNS(=O)(=O)c1ccc(N2C(=O)C=CC2=O)cc1] |
| 2324 | 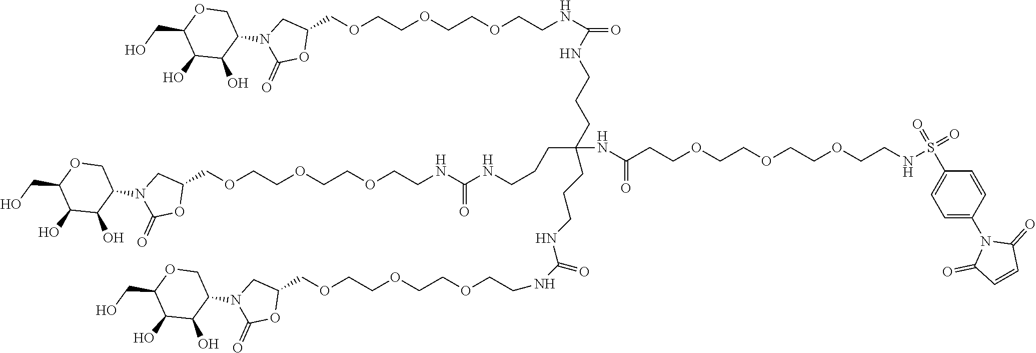 [SMILES: O=C(CCOCCOCCOCCNS(=O)(=O)c1ccc(N2C(=O)C=CC2=O)cc1)NC(CCCNC(=O)NCCOCCOCCOC[C@H]1CN([C@H]2CO[C@H](CO)[C@H](O)[C@@H]2O)C(=O)O1)(CCCNC(=O)NCCOCCOCCOC[C@H]1CN([C@H]2CO[C@H](CO)[C@H](O)[C@@H]2O)C(=O)O1)CCCNC(=O)NCCOCCOCCOC[C@H]1CN([C@H]2CO[C@H](CO)[C@H](O)[C@@H]2O)C(=O)O1] |
| 2325 | 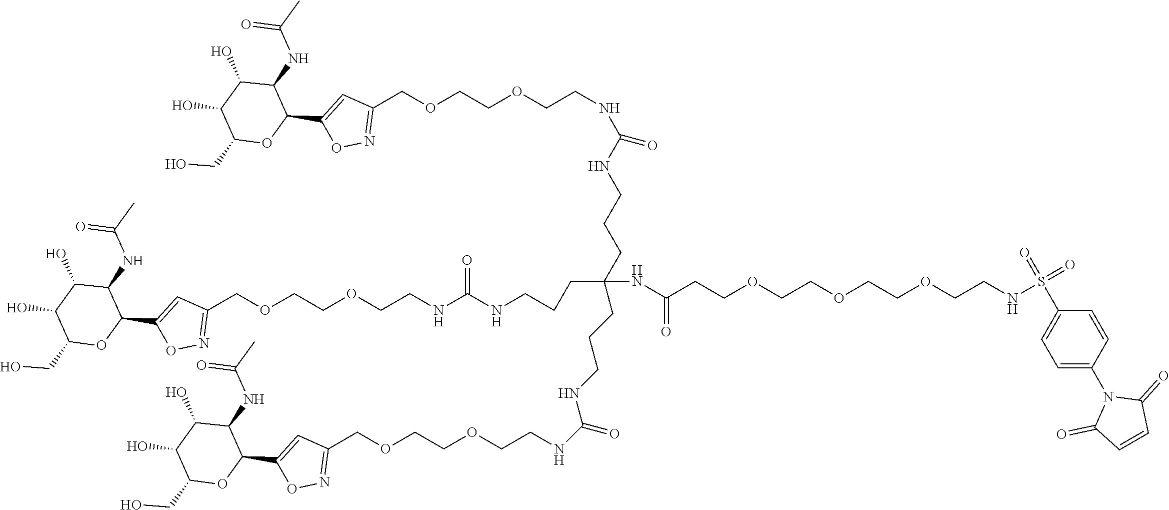 [SMILES: CC(=O)N[C@@H]1[C@@H](O)[C@@H](O)[C@@H](CO)O[C@@H]1c1cc(COCCOCCNC(=O)NCCCC(CCCNC(=O)NCCOCCOCc2cc([C@H]3O[C@H](CO)[C@H](O)[C@H](O)[C@H]3NC(C)=O)on2)(CCCNC(=O)NCCOCCOCc2cc([C@H]3O[C@H](CO)[C@H](O)[C@H](O)[C@H]3NC(C)=O)on2)NC(=O)CCOCCOCCOCCNS(=O)(=O)c2ccc(N3C(=O)C=CC3=O)cc2)no1] |
| 2326 | 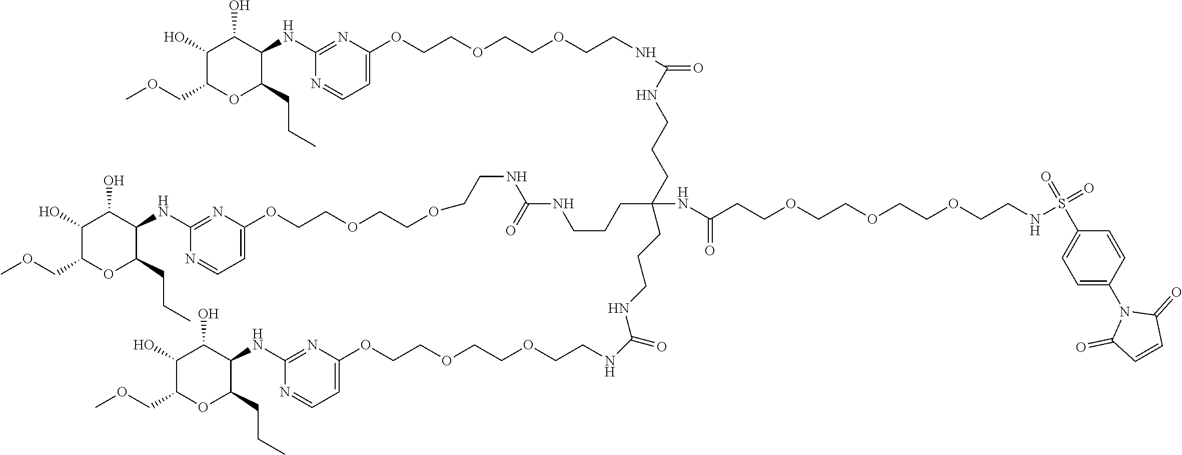 [SMILES: CCC[C@H]1O[C@H](COC)[C@H](O)[C@H](O)[C@H]1Nc1nccc(OCCOCCOCCNC(=O)NCCCC(CCCNC(=O)NCCOCCOCCOc2ccnc(N[C@@H]3[C@@H](O)[C@@H](O)[C@@H](COC)O[C@@H]3CCC)n2)(CCCNC(=O)NCCOCCOCCOc2ccnc(N[C@@H]3[C@@H](O)[C@@H](O)[C@@H](COC)O[C@@H]3CCC)n2)NC(=O)CCOCCOCCOCCNS(=O)(=O)c2ccc(N3C(=O)C=CC3=O)cc2)n1] |
| 2327 | 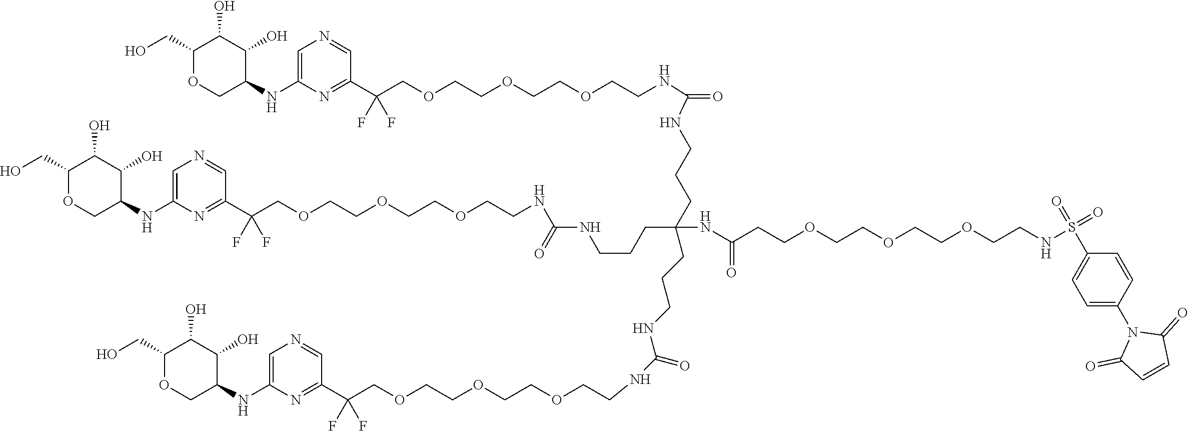 [SMILES: O=C(CCOCCOCCOCCNS(=O)(=O)c1ccc(N2C(=O)C=CC2=O)cc1)NC(CCCNC(=O)NCCOCCOCCOCC(F)(F)c1cncc(N[C@H]2CO[C@H](CO)[C@H](O)[C@@H]2O)n1)(CCCNC(=O)NCCOCCOCCOCC(F)(F)c1cncc(N[C@H]2CO[C@H](CO)[C@H](O)[C@@H]2O)n1)CCCNC(=O)NCCOCCOCCOCC(F)(F)c1cncc(N[C@H]2CO[C@H](CO)[C@H](O)[C@@H]2O)n1] |
| 2328 | 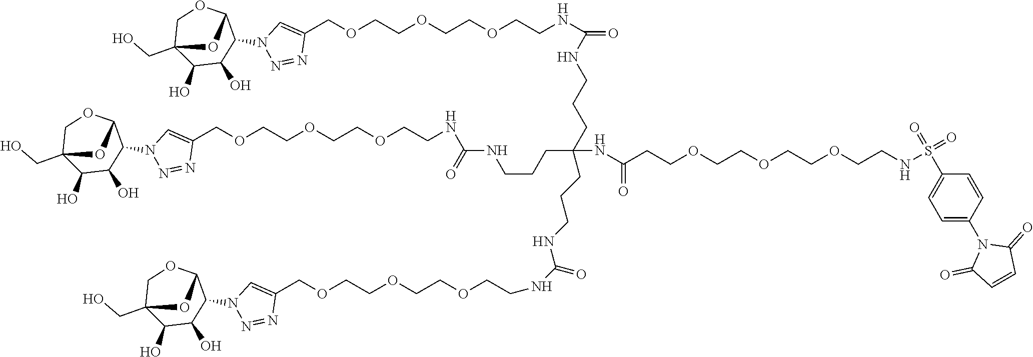 [SMILES: O=C(CCOCCOCCOCCNS(=O)(=O)c1ccc(N2C(=O)C=CC2=O)cc1)NC(CCCNC(=O)NCCOCCOCCOCc1cn([C@H]2[C@H]3OC[C@](CO)(O3)[C@H](O)[C@@H]2O)nn1)(CCCNC(=O)NCCOCCOCCOCc1cn([C@H]2[C@H]3OC[C@](CO)(O3)[C@H](O)[C@@H]2O)nn1)CCCNC(=O)NCCOCCOCCOCc1cn([C@H]2[C@H]3OC[C@](CO)(O3)[C@H](O)[C@@H]2O)nn1] |
| 2329 | 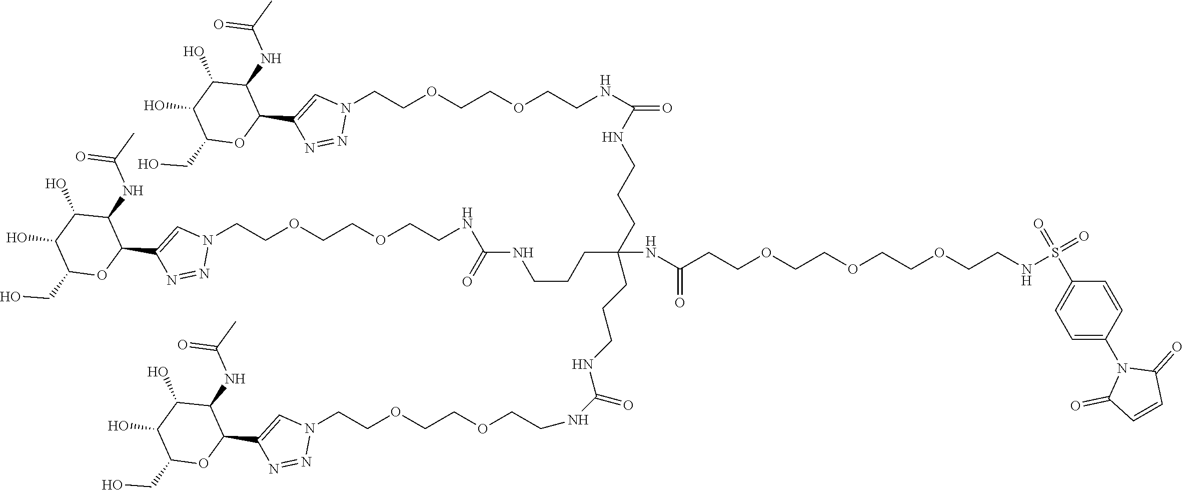 [SMILES: CC(=O)N[C@@H]1[C@@H](O)[C@@H](O)[C@@H](CO)O[C@@H]1c1cn(CCOCCOCCNC(=O)NCCCC(CCCNC(=O)NCCOCCOCCn2cc([C@H]3O[C@H](CO)[C@H](O)[C@H](O)[C@H]3NC(C)=O)nn2)(CCCNC(=O)NCCOCCOCCn2cc([C@H]3O[C@H](CO)[C@H](O)[C@H](O)[C@H]3NC(C)=O)nn2)NC(=O)CCOCCOCCOCCNS(=O)(=O)c2ccc(N3C(=O)C=CC3=O)cc2)nn1] |
| 2330 | 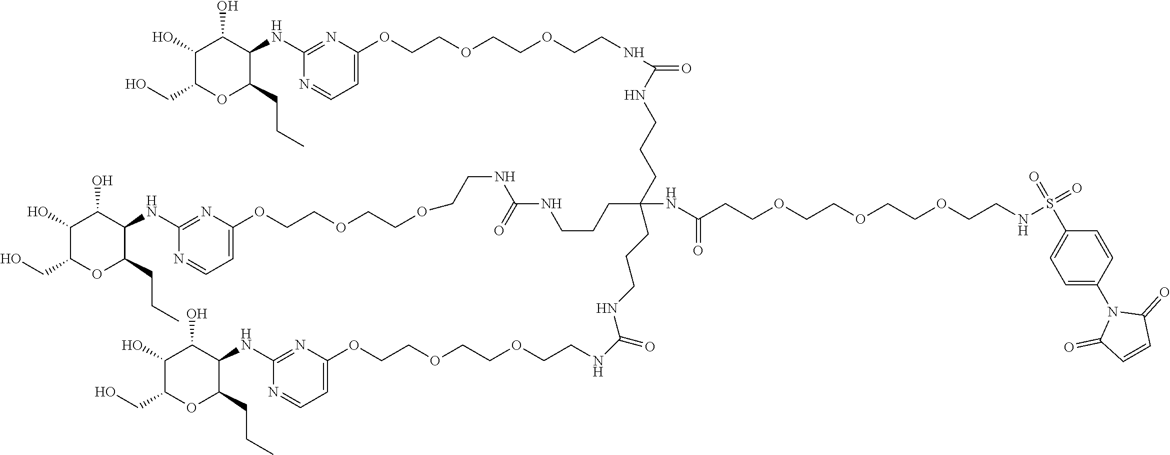 [SMILES: CCC[C@H]1O[C@H](CO)[C@H](O)[C@H](O)[C@H]1Nc1nccc(OCCOCCOCCNC(=O)NCCCC(CCCNC(=O)NCCOCCOCCOc2ccnc(N[C@@H]3[C@@H](O)[C@@H](O)[C@@H](CO)O[C@@H]3CCC)n2)(CCCNC(=O)NCCOCCOCCOc2ccnc(N[C@@H]3[C@@H](O)[C@@H](O)[C@@H](CO)O[C@@H]3CCC)n2)NC(=O)CCOCCOCCOCCNS(=O)(=O)c2ccc(N3C(=O)C=CC3=O)cc2)n1] |
| 2331 | 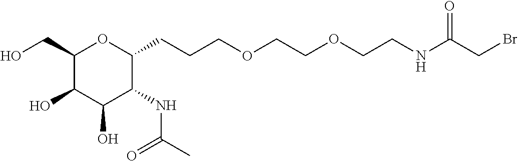 [SMILES: CC(=O)N[C@@H]1[C@@H](O)[C@@H](O)[C@@H](CO)O[C@@H]1CCCOCCOCCNC(=O)CBr] |
| 2332 | 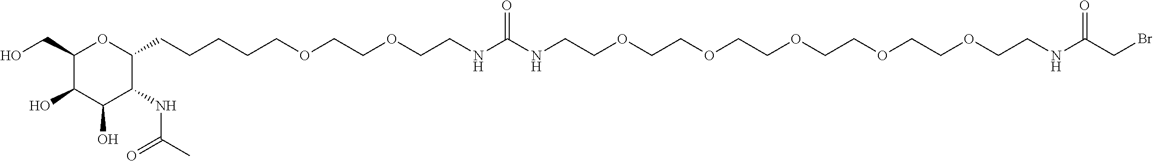 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)CBr] |
| 2333 | 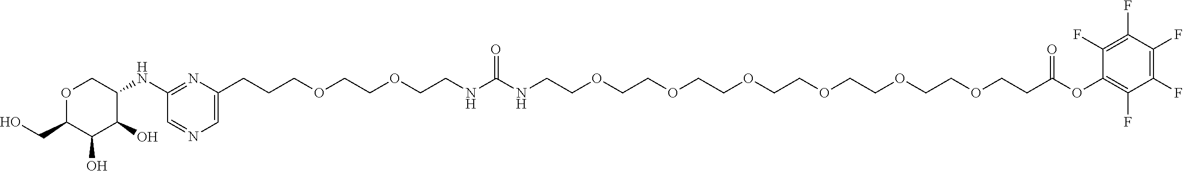 [SMILES: O=C(NCCOCCOCCCc1cncc(N[C@H]2CO[C@H](CO)[C@H](O)[C@@H]2O)n1)NCCOCCOCCOCCOCCOCCOCCC(=O)Oc1c(F)c(F)c(F)c(F)c1F] |
| 2334 | 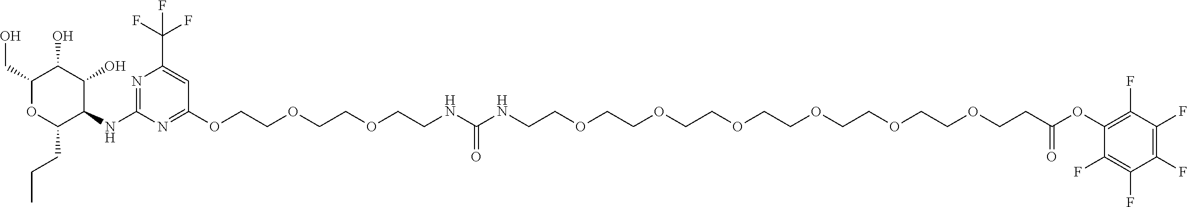 [SMILES: CCC[C@@H]1O[C@H](CO)[C@H](O)[C@H](O)[C@H]1Nc1nc(OCCOCCOCCNC(=O)NCCOCCOCCOCCOCCOCCOCCC(=O)Oc2c(F)c(F)c(F)c(F)c2F)cc(C(F)(F)F)n1] |
| 2335 | 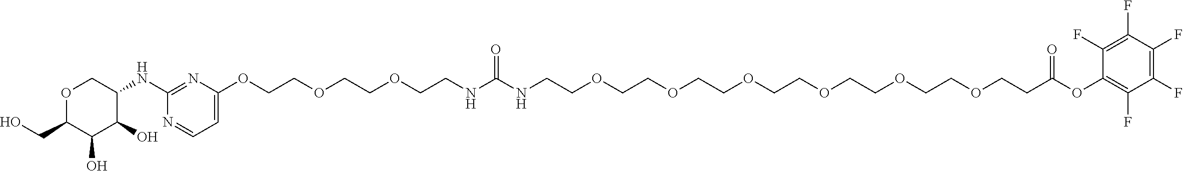 [SMILES: O=C(NCCOCCOCCOCCOCCOCCOCCC(=O)Oc1c(F)c(F)c(F)c(F)c1F)NCCOCCOCCOc1ccnc(N[C@H]2CO[C@H](CO)[C@H](O)[C@@H]2O)n1] |
| 2336 | 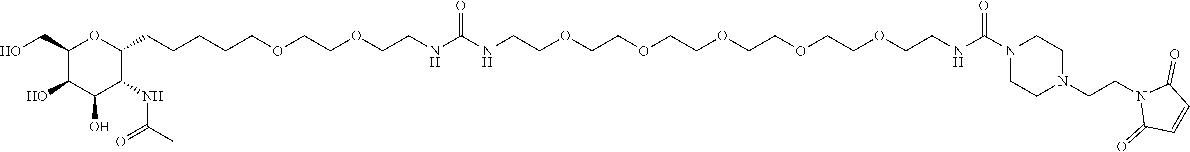 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)N1CCN(CCN2C(=O)C=CC2=O)CC1] |
| 2337 | 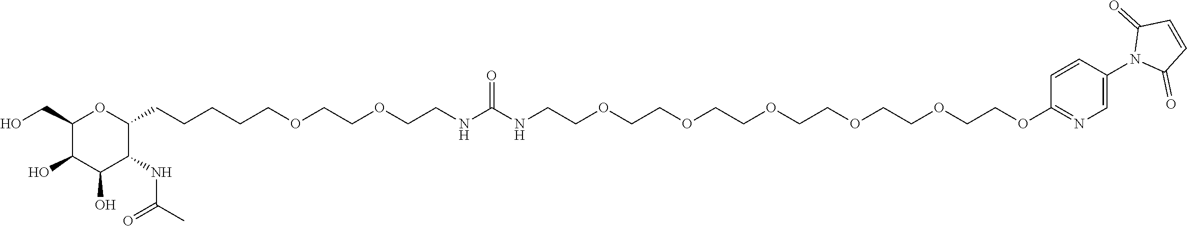 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOc1ccc(N2C(=O)C=CC2=O)cn1] |
| 2338 | 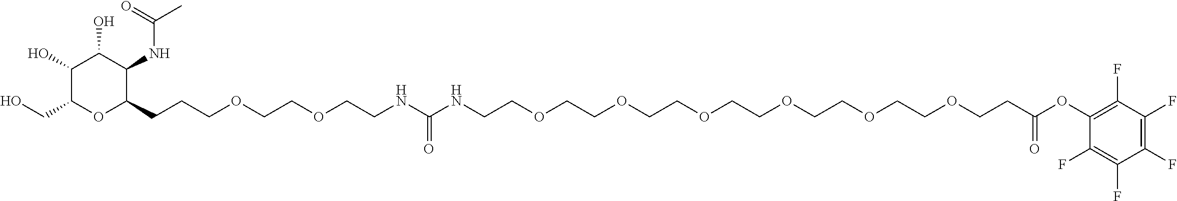 [SMILES: CC(=O)N[C@@H]1[C@@H](O)[C@@H](O)[C@@H](CO)O[C@@H]1CCCOCCOCCNC(=O)NCCOCCOCCOCCOCCOCCOCCC(=O)Oc1c(F)c(F)c(F)c(F)c1F] |
| 2339 | 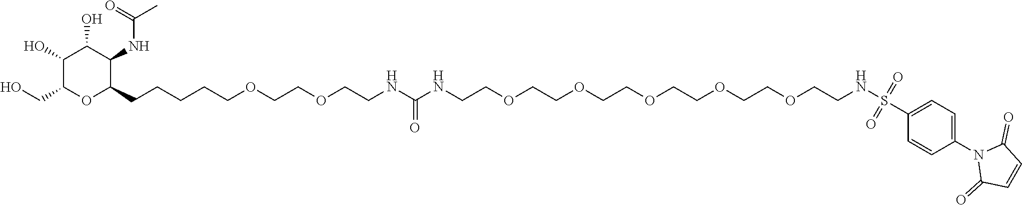 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNS(=O)(=O)c1ccc(N2C(=O)C=CC2=O)cc1] |
| 2340 | 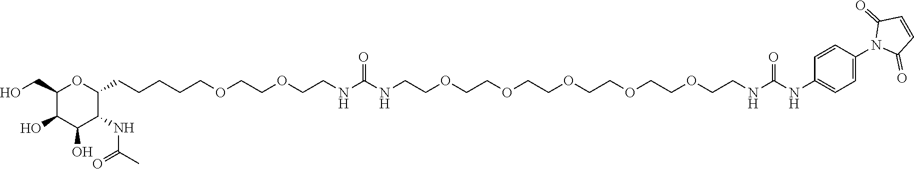 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)Nc1ccc(N2C(=O)C=CC2=O)cc1] |
| 2341 | 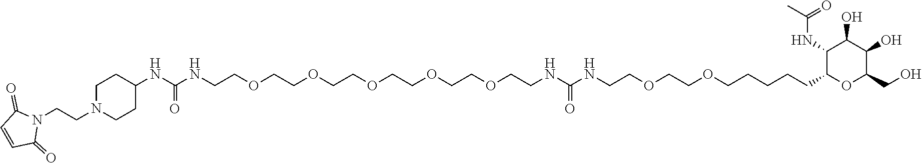 [SMILES: CC(=O)N[C@@H]1[C@@H](O)[C@@H](O)[C@@H](CO)O[C@@H]1CCCCCOCCOCCNC(=O)NCCOCCOCCOCCOCCOCCNC(=O)NC1CCN(CCN2C(=O)C=CC2=O)CC1] |
| 2342 | 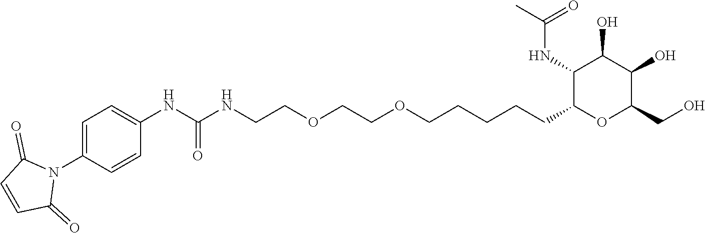 [SMILES: CC(=O)N[C@@H]1[C@@H](O)[C@@H](O)[C@@H](CO)O[C@@H]1CCCCCOCCOCCNC(=O)Nc1ccc(N2C(=O)C=CC2=O)cc1] |
| 2343 | 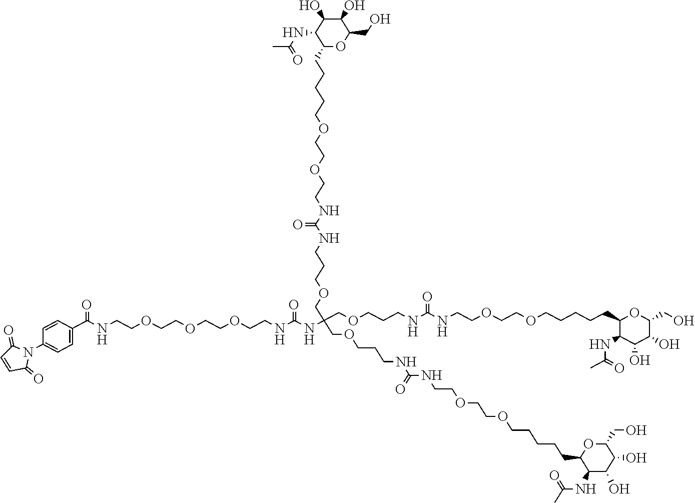 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)(COCCCNC(=O)NCCOCCOCCCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2344 | 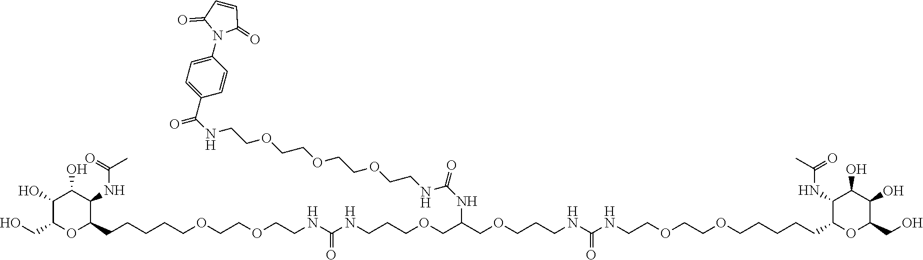 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)NC(=O)NCCOCCOCCOCCNC(=O)c1ccc(N2C(=O)C=CC2=O)cc1] |
| 2345 | 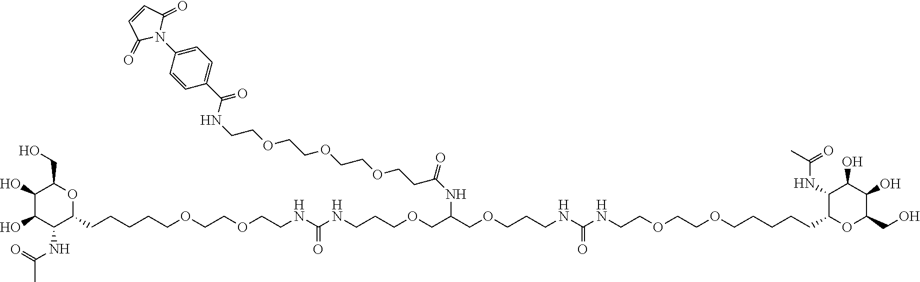 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)NC(=O)CCOCCOCCOCCNC(=O)c1ccc(N2C(=O)C=CC2=O)cc1] |
| 2346 | 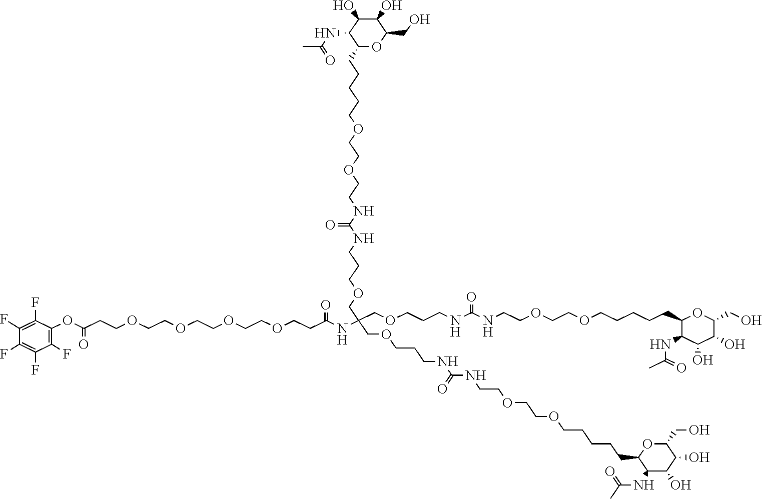 [SMILES: CC(=O)N[C@@H]1[C@@H](O)[C@@H](O)[C@@H](CO)O[C@@H]1CCCCCOCCOCCNC(=O)NCCCOCC(COCCCNC(=O)NCCOCCOCCCCC[C@H]1O[C@H](CO)[C@H](O)[C@H](O)[C@H]1NC(C)=O)(COCCCNC(=O)NCCOCCOCCCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2347 | 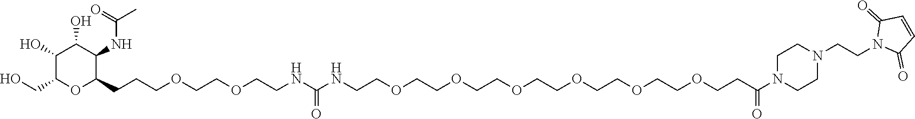 [SMILES: CC(=O)N[C@@H]1[C@@H](O)[C@@H](O)[C@@H](CO)O[C@@H]1CCCOCCOCCNC(=O)NCCOCCOCCOCCOCCOCCOCCC(=O)N1CCN(CCN2C(=O)C=CC2=O)CC1] |
| 2348 | 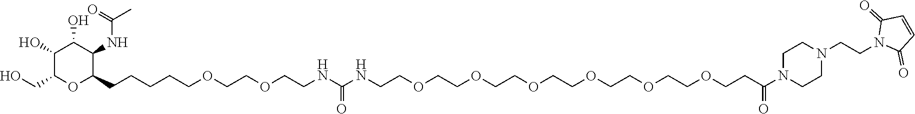 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)N1CCN(CCN2C(=O)C=CC2=O)CC1] |
| 2349 | 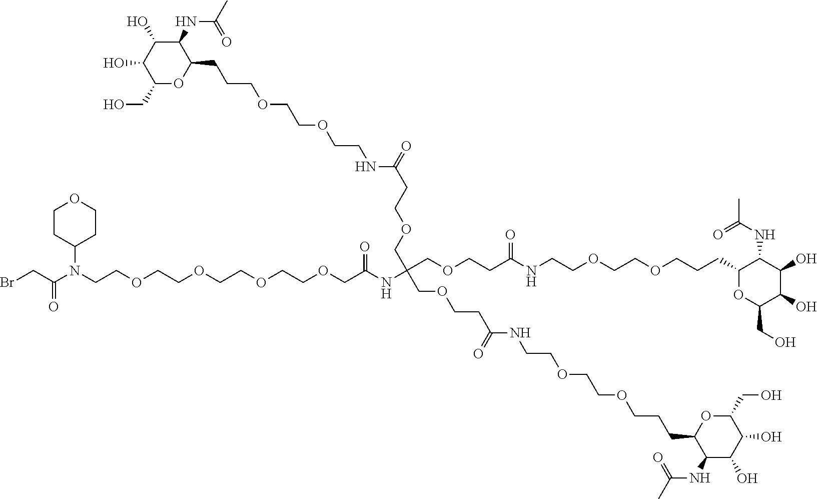 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)COCCOCCOCCOCCN(C(=O)CBr)C1CCOCC1] |
| 2350 | 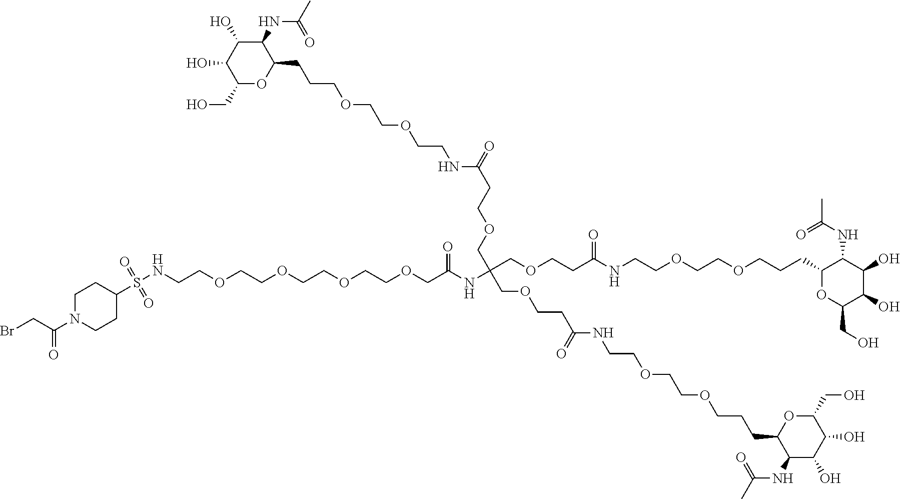 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)COCCOCCOCCOCCNS(=O)(=O)C1CCN(C(=O)CBr)CC1] |
| 2351 | 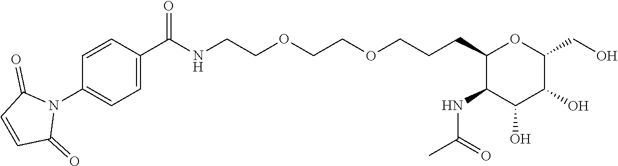 [SMILES: CC(=O)N[C@@H]1[C@@H](O)[C@@H](O)[C@@H](CO)O[C@@H]1CCCOCCOCCNC(=O)c1ccc(N2C(=O)C=CC2=O)cc1] |
| 2352 | 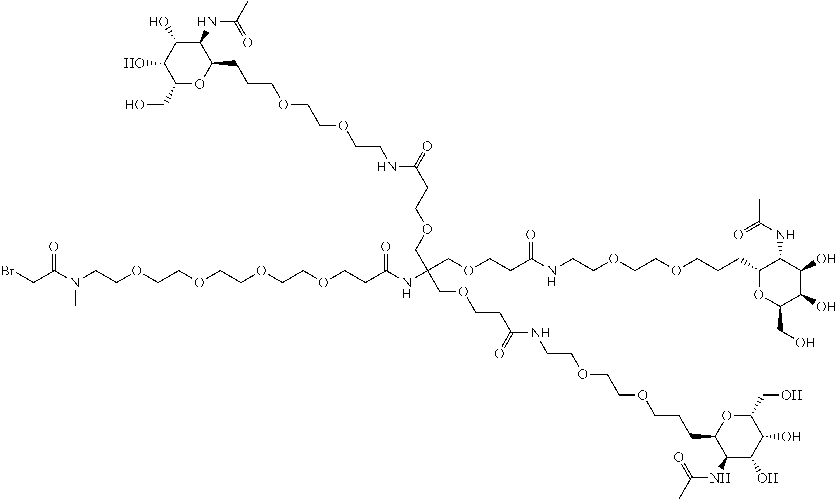 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCN(C)C(=O)CBr] |
| 2353 | 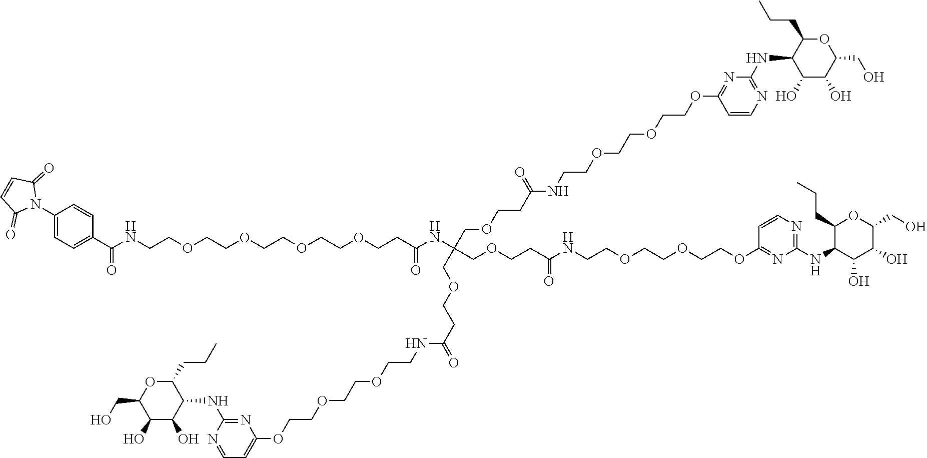 [SMILES: CCC[C@H]1O[C@H](CO)[C@H](O)[C@H](O)[C@H]1Nc1nccc(OCCOCCOCCNC(=O)CCOCC(COCCC(=O)NCCOCCOCCOc2ccnc(N[C@@H]3[C@@H](O)[C@@H](O)[C@@H](CO)O[C@@H]3CCC)n2)(COCCC(=O)NCCOCCOCCOc2ccnc(N[C@@H]3[C@@H](O)[C@@H](O)[C@@H](CO)O[C@@H]3CCC)n2)NC(=O)CCOCCOCCOCCOCCNC(=O)c2ccc(N3C(=O)C=CC3=O)cc2)n1] |
| 2354 | 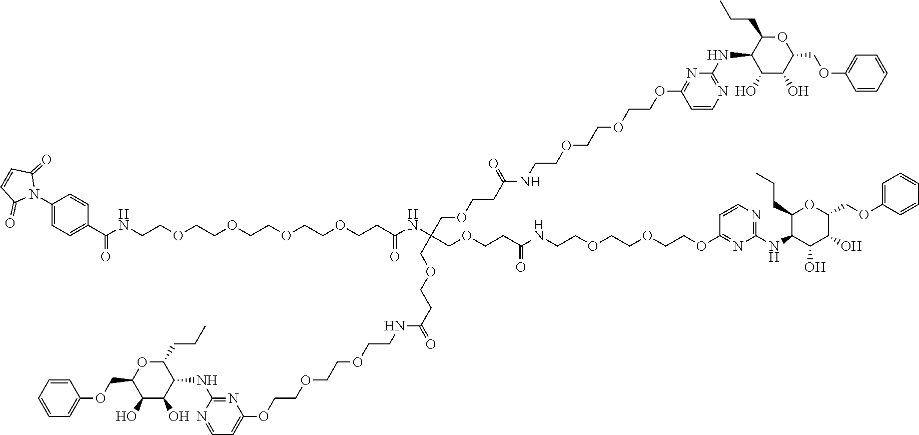 [SMILES: CCC[C@H]1O[C@H](COc2ccccc2)[C@H](O)[C@H](O)[C@H]1Nc1nccc(OCCOCCOCCNC(=O)CCOCC(COCCC(=O)NCCOCCOCCOc2ccnc(N[C@@H]3[C@@H](O)[C@@H](O)[C@@H](COc4ccccc4)O[C@@H]3CCC)n2)(COCCC(=O)NCCOCCOCCOc2ccnc(N[C@@H]3[C@@H](O)[C@@H](O)[C@@H](COc4ccccc4)O[C@@H]3CCC)n2)NC(=O)CCOCCOCCOCCOCCNC(=O)c2ccc(N3C(=O)C=CC3=O)cc2)n1] |
| 2355 | 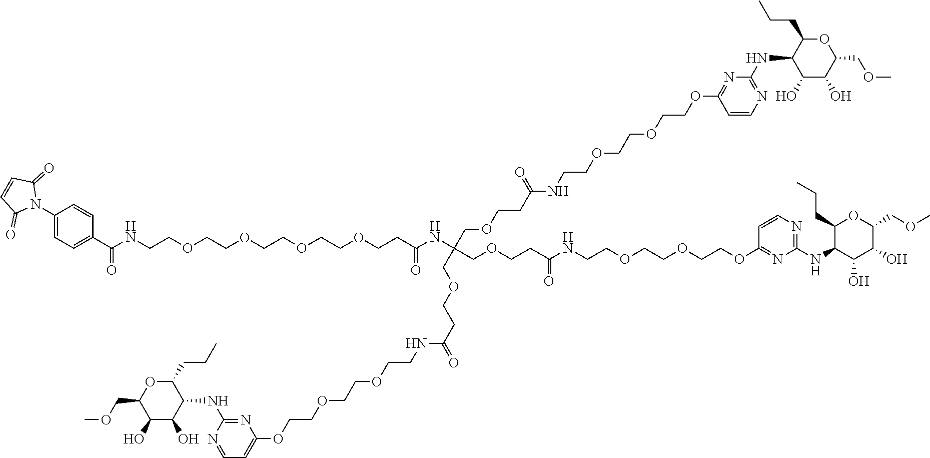 [SMILES: CCC[C@H]1O[C@H](COC)[C@H](O)[C@H](O)[C@H]1Nc1nccc(OCCOCCOCCNC(=O)CCOCC(COCCC(=O)NCCOCCOCCOc2ccnc(N[C@@H]3[C@@H](O)[C@@H](O)[C@@H](COC)O[C@@H]3CCC)n2)(COCCC(=O)NCCOCCOCCOc2ccnc(N[C@@H]3[C@@H](O)[C@@H](O)[C@@H](COC)O[C@@H]3CCC)n2)NC(=O)CCOCCOCCOCCOCCNC(=O)c2ccc(N3C(=O)C=CC3=O)cc2)n1] |
| 2356 | 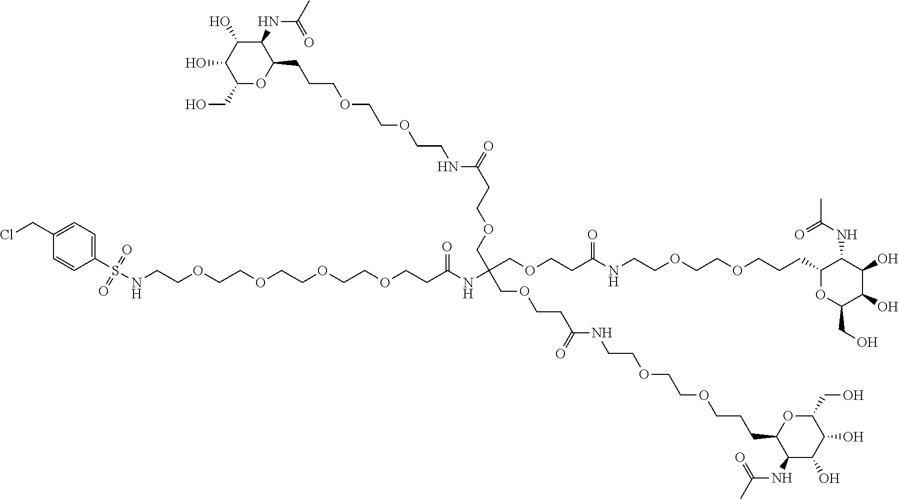 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNS(=O)(=O)c1ccc(CCl)cc1] |
| 2357 | 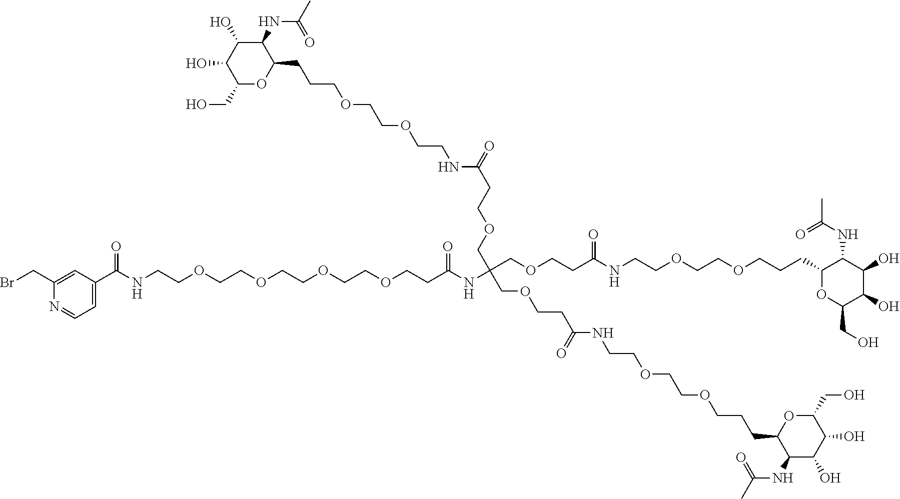 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)c1ccnc(CBr)c1] |
| 2358 | 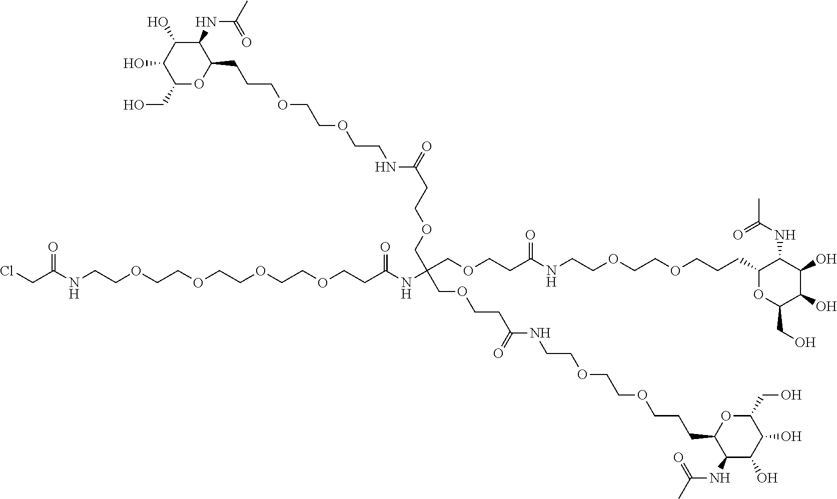 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CCl] |
| 2359 | 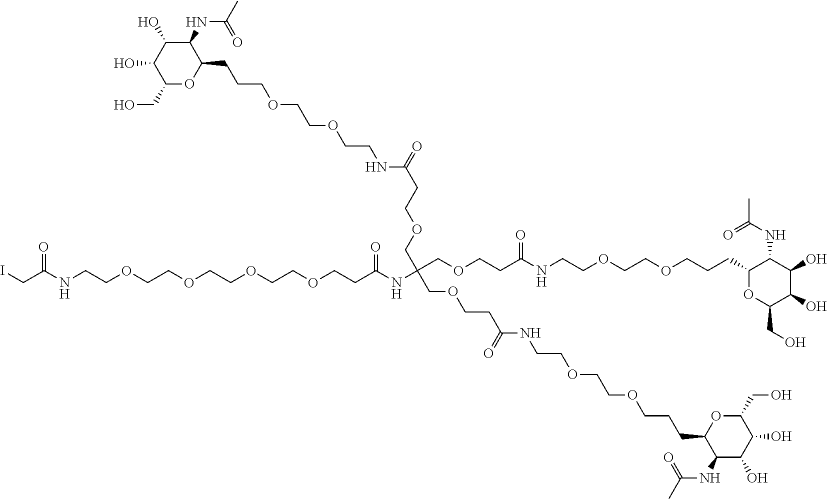 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CI] |
| 2360 | 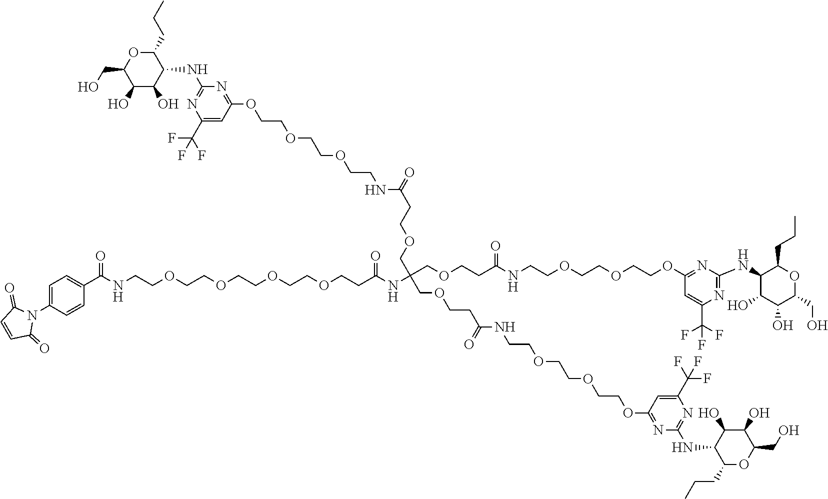 [SMILES: CCC[C@H]1O[C@H](CO)[C@H](O)[C@H](O)[C@H]1Nc1nc(OCCOCCOCCNC(=O)CCOCC(COCCC(=O)NCCOCCOCCOc2cc(C(F)(F)F)nc(N[C@@H]3[C@@H](O)[C@@H](O)[C@@H](CO)O[C@@H]3CCC)n2)(COCCC(=O)NCCOCCOCCOc2cc(C(F)(F)F)nc(N[C@@H]3[C@@H](O)[C@@H](O)[C@@H](CO)O[C@@H]3CCC)n2)NC(=O)CCOCCOCCOCCOCCNC(=O)c2ccc(N3C(=O)C=CC3=O)cc2)cc(C(F)(F)F)n1] |
| 2361 | 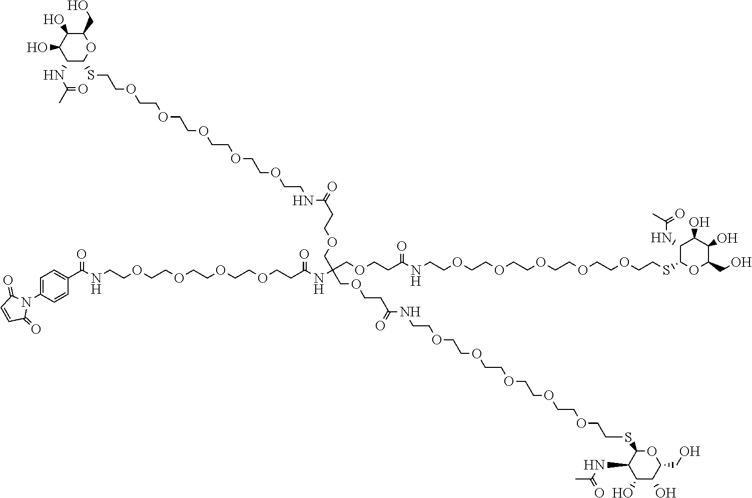 [SMILES: CC(=O)N[C@@H]1[C@@H](O)[C@@H](O)[C@@H](CO)O[C@@H]1SCCOCCOCCOCCOCCOCCNC(=O)CCOCC(COCCC(=O)NCCOCCOCCOCCOCCOCCS[C@H]1O[C@H](CO)[C@H](O)[C@H](O)[C@H]1NC(C)=O)(COCCC(=O)NCCOCCOCCOCCOCCOCCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2362 | 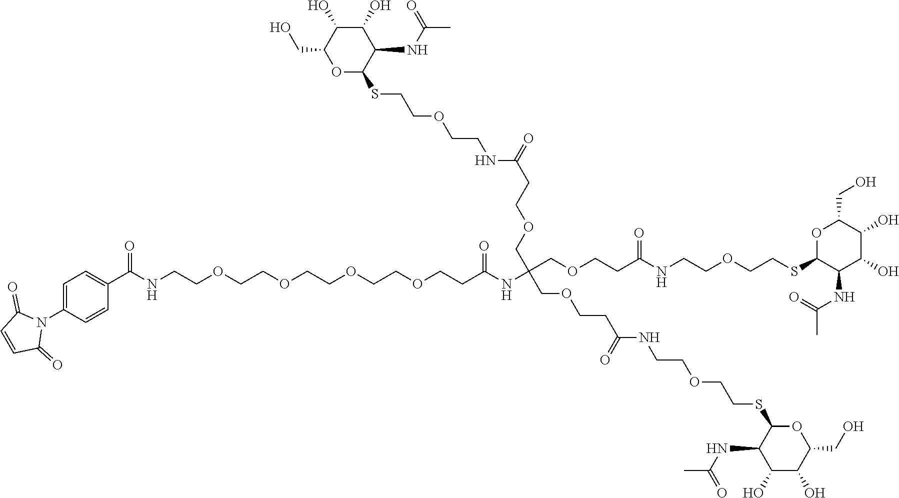 [SMILES: CC(=O)N[C@@H]1[C@@H](O)[C@@H](O)[C@@H](CO)O[C@@H]1SCCOCCNC(=O)CCOCC(COCCC(=O)NCCOCCS[C@H]1O[C@H](CO)[C@H](O)[C@H](O)[C@H]1NC(C)=O)(COCCC(=O)NCCOCCS[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2363 | 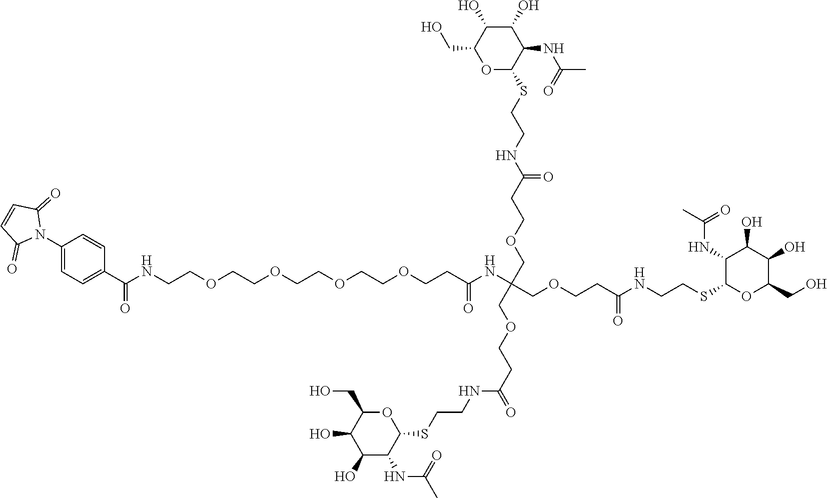 [SMILES: CC(=O)N[C@@H]1[C@@H](O)[C@@H](O)[C@@H](CO)O[C@@H]1SCCNC(=O)CCOCC(COCCC(=O)NCCS[C@H]1O[C@H](CO)[C@H](O)[C@H](O)[C@H]1NC(C)=O)(COCCC(=O)NCCS[C@@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1] |
| 2364 | 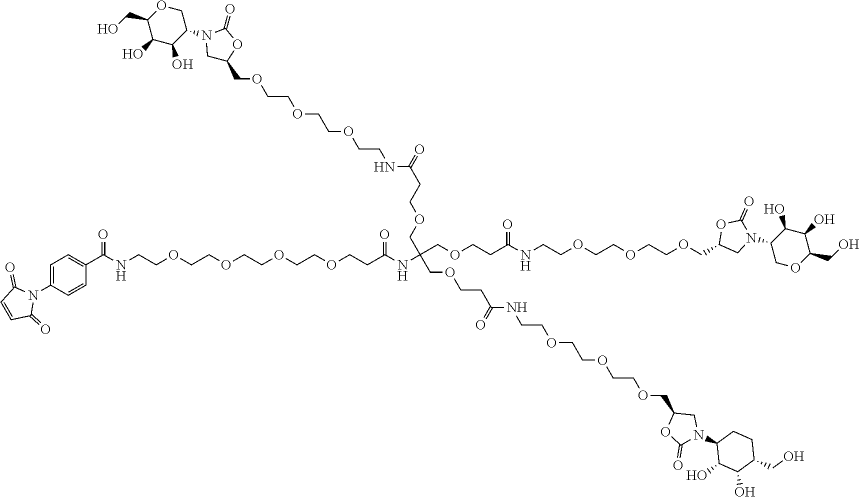 [SMILES: O=C(CCOCC(COCCC(=O)NCCOCCOCCOC[C@H]1CN([C@H]2CO[C@H](CO)[C@H](O)[C@@H]2O)C(=O)O1)(COCCC(=O)NCCOCCOCCOC[C@H]1CN([C@H]2CC[C@H](CO)[C@H](O)[C@@H]2O)C(=O)O1)NC(=O)CCOCCOCCOCCOCCNC(=O)c1ccc(N2C(=O)C=CC2=O)cc1)NCCOCCOCCOC[C@H]1CN([C@H]2CO[C@H](CO)[C@H](O)[C@@H]2O)C(=O)O1] |
| 2365 | 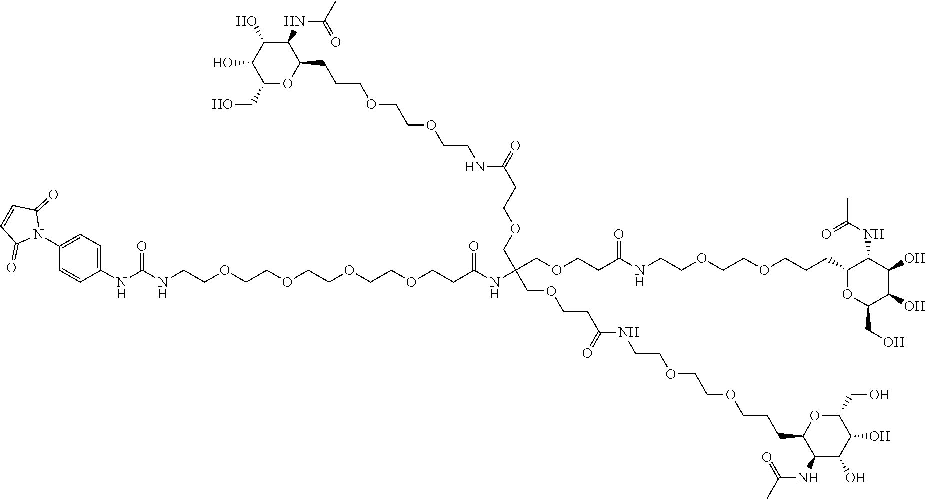 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)Nc1ccc(N2C(=O)C=CC2=O)cc1] |
| 2366 | 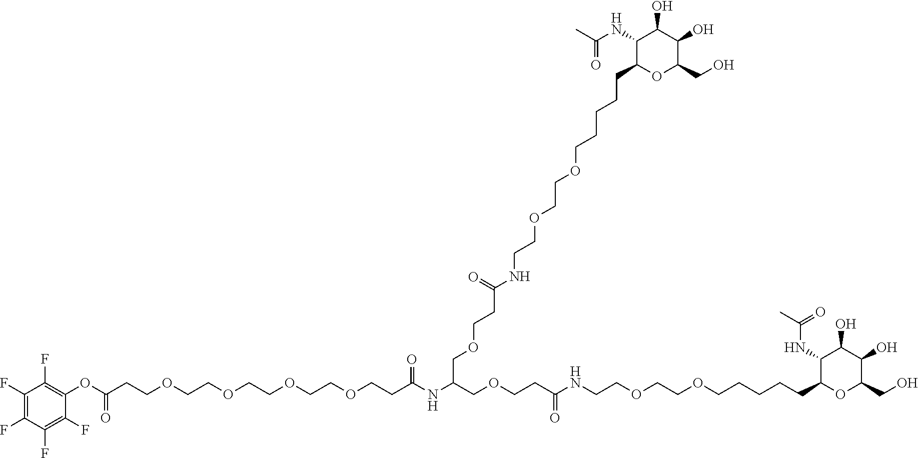 [SMILES: CC(=O)N[C@@H]1[C@@H](O)[C@@H](O)[C@@H](CO)O[C@H]1CCCCCOCCOCCNC(=O)CCOCC(COCCC(=O)NCCOCCOCCCCC[C@@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2367 | 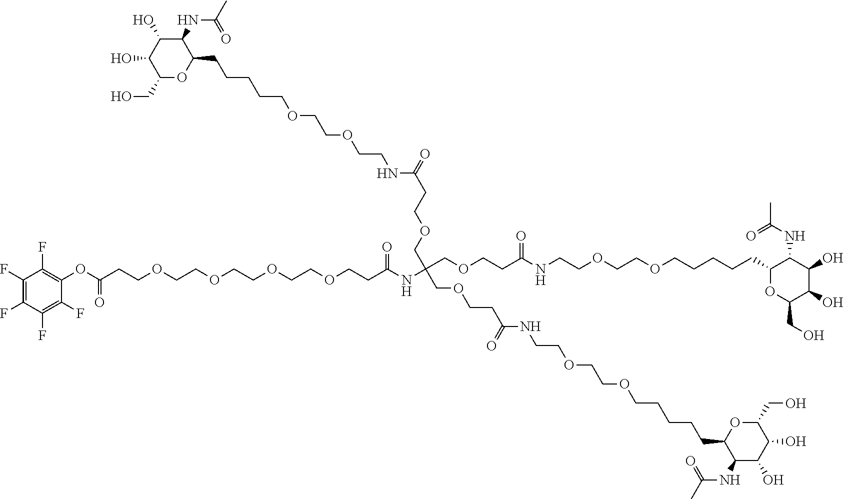 [SMILES: CC(=O)N[C@@H]1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)[C@H]1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2368 | 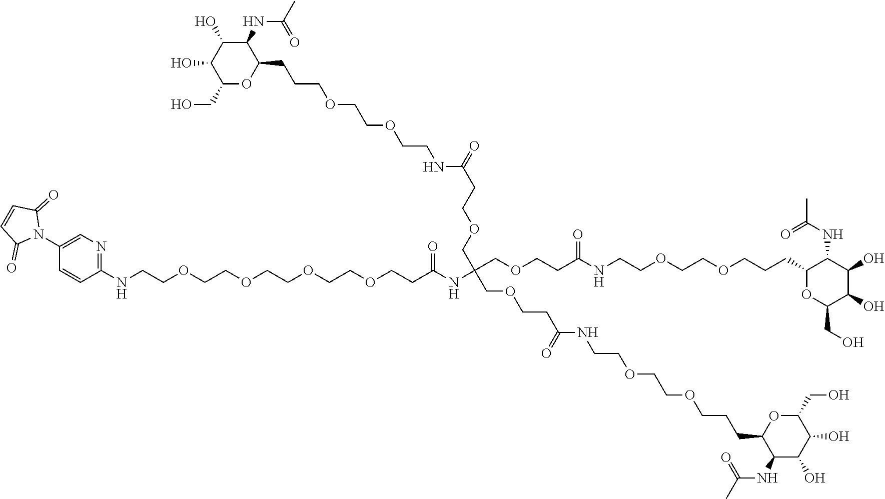 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNc1ccc(N2C(=O)C=CC2=O)cn1] |
| 2369 | 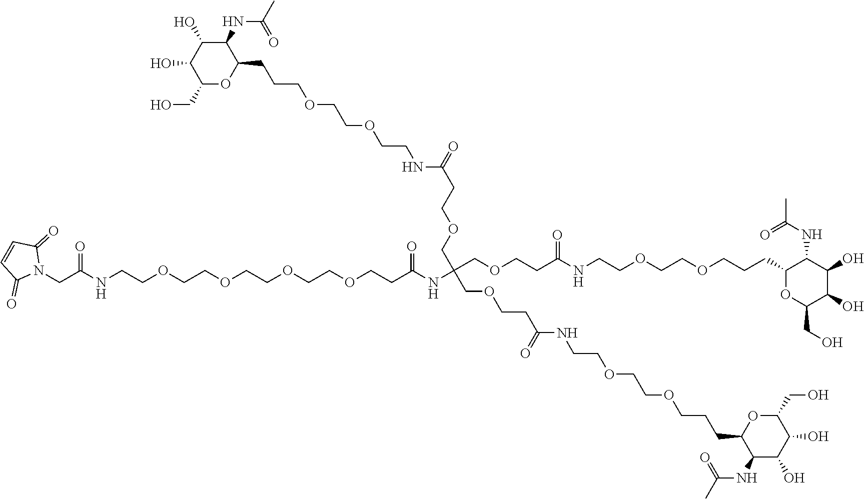 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CN1C(=O)C=CC1=O] |
| 2370 | 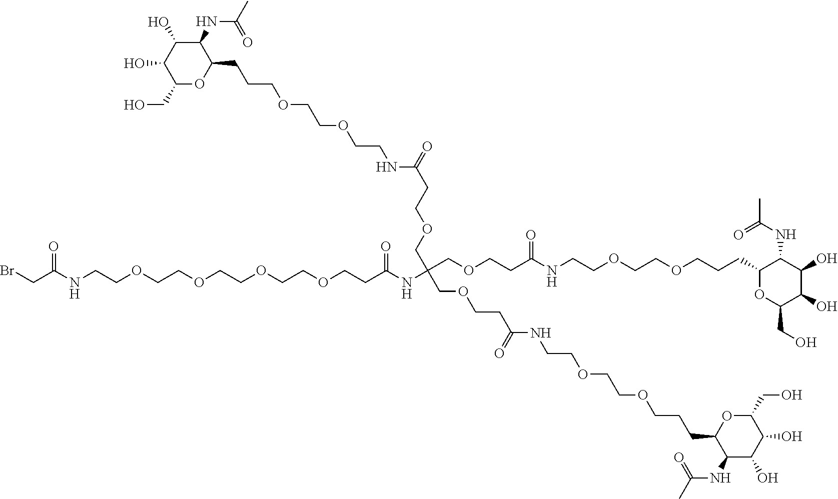 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)CBr] |
| 2371 | 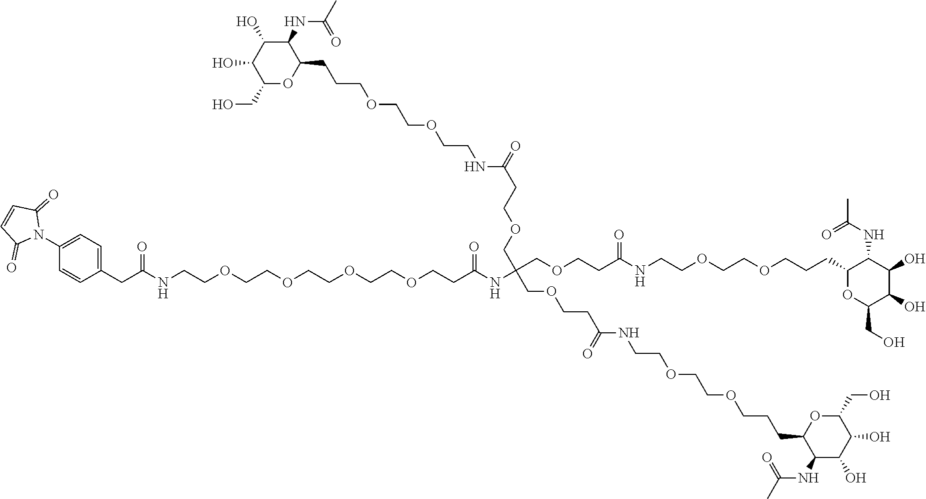 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)Cc1ccc(N2C(=O)C=CC2=O)cc1] |
| 2372 | 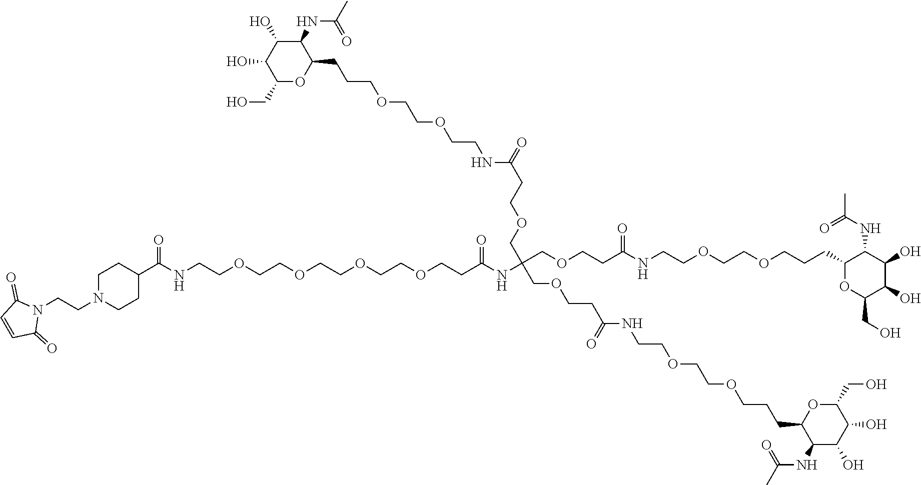 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)C1CCN(CCN2C(=O)C=CC2=O)CC1] |
| 2373 | 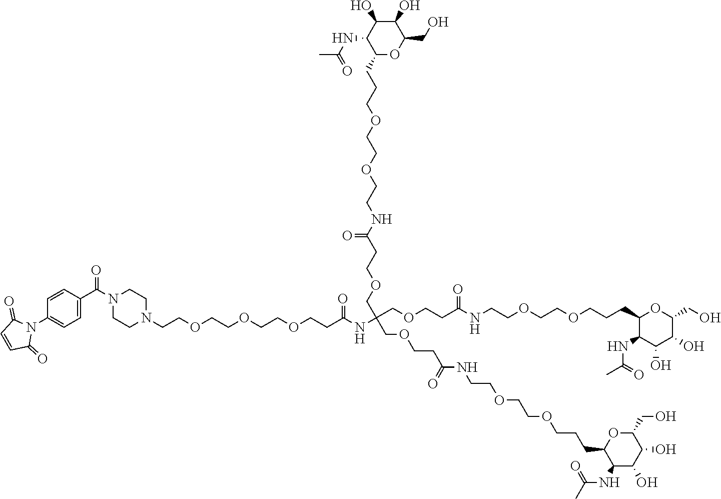 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCN1CCN(C(=O)c2ccc(N3C(=O)C=CC3=O)cc2)CC1] |
| 2374 | 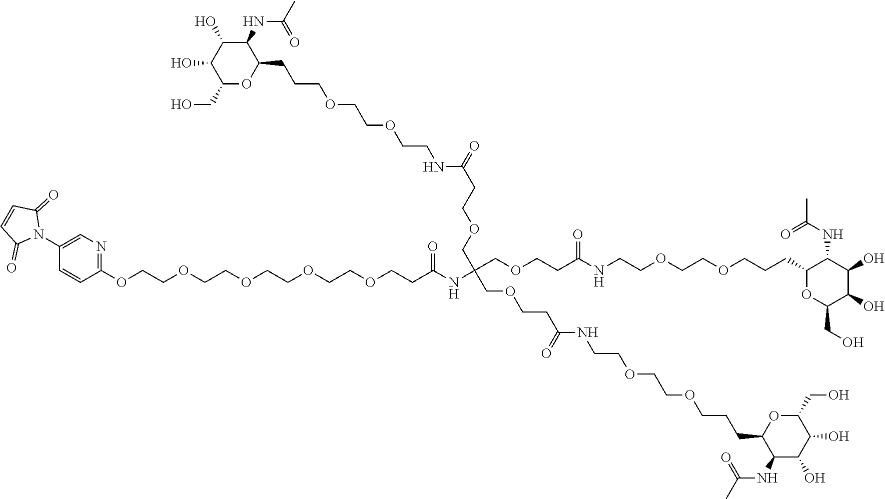 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCOc1ccc(N2C(=O)C=CC2=O)cn1] |
| 2375 | 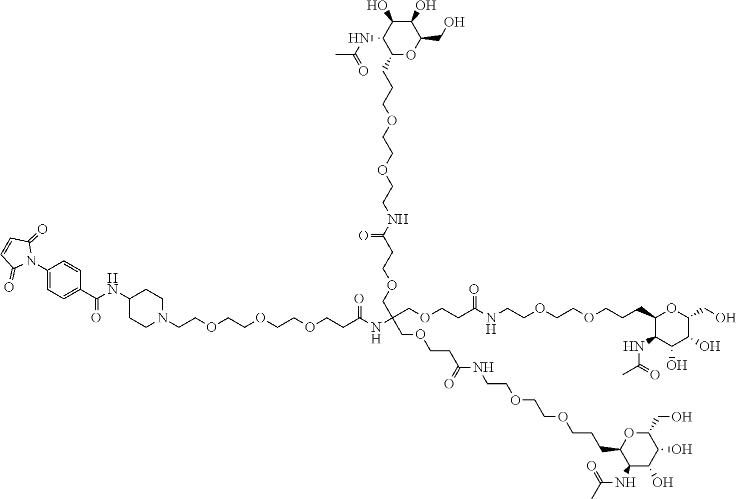 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCN1CCC(NC(=O)c2ccc(N3C(=O)C=CC3=O)cc2)CC1] |
| 2376 | 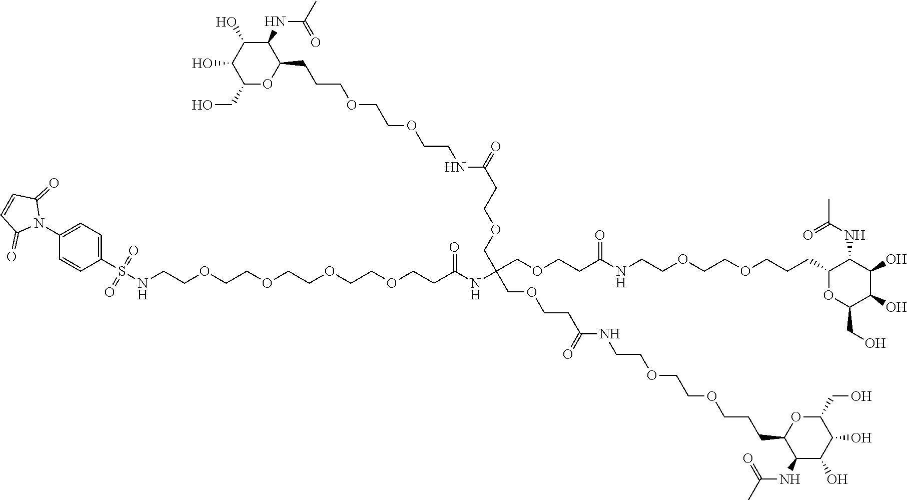 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNS(=O)(=O)c1ccc(N2C(=O)C=CC2=O)cc1] |
| 2377 | 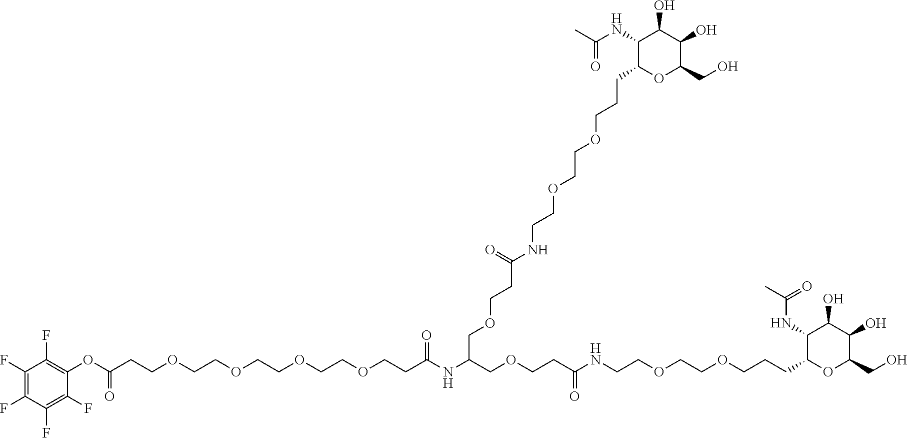 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2378 | 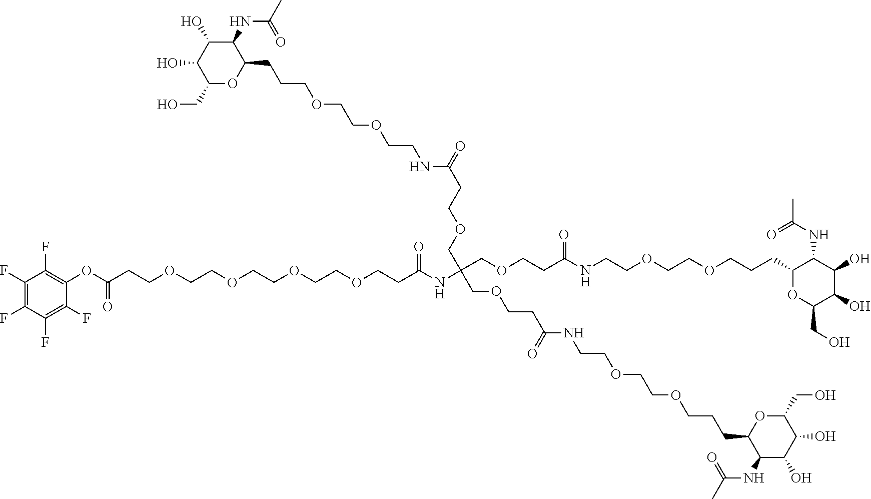 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCC(=O)Oc1c(F)c(F)c(F)c(F)c1F] |
| 2379 | 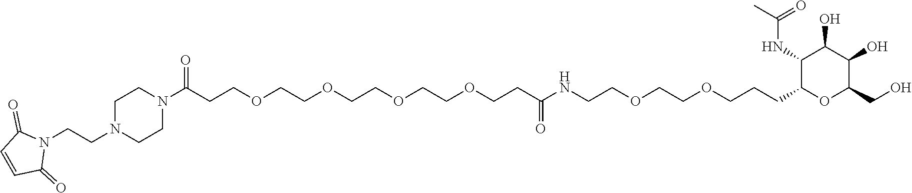 [SMILES: CC(=O)N[C@@H]1[C@@H](O)[C@@H](O)[C@@H](CO)O[C@@H]1CCCOCCOCCNC(=O)CCOCCOCCOCCOCCC(=O)N1CCN(CCN2C(=O)C=CC2=O)CC1] |
| 2380 | 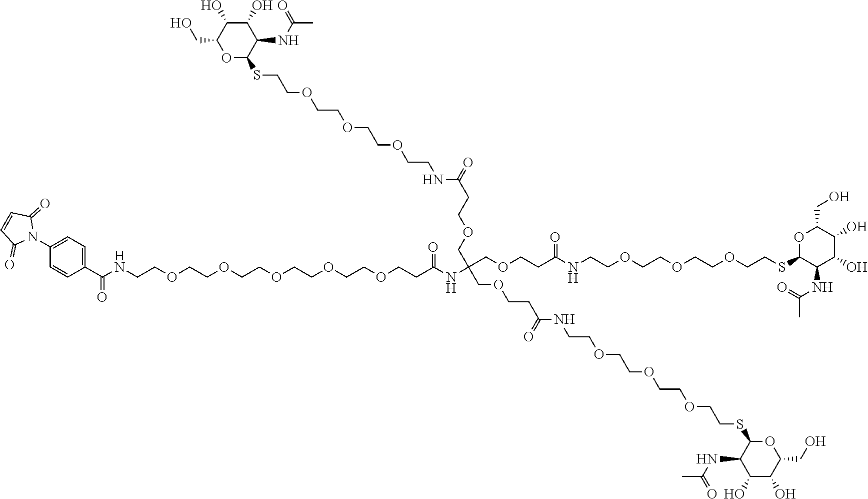 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCOCCOCCOCCNC(=O)c1ccc(N2C(=O)C=CC2=O)cc1] |
| 2381 | 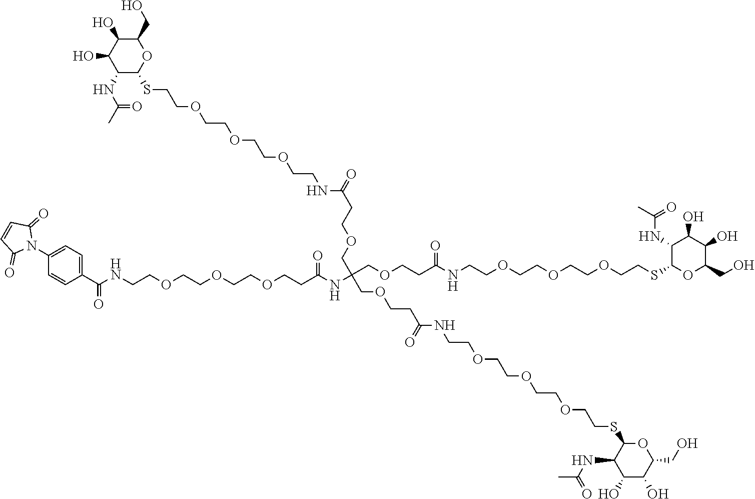 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCOCCOCCNC(=O)c1ccc(N2C(=O)C=CC2=O)cc1] |
| 2382 | 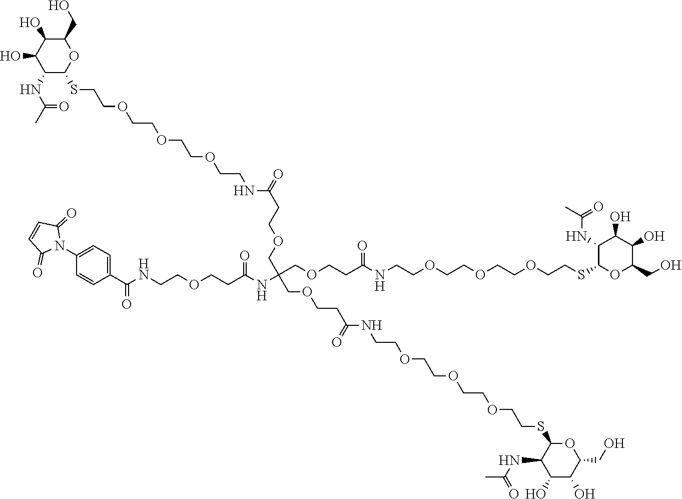 [SMILES: CC(=O)N[C@@H]1[C@@H](O)[C@@H](O)[C@@H](CO)O[C@@H]1SCCOCCOCCOCCNC(=O)CCOCC(COCCC(=O)NCCOCCOCCOCCS[C@H]1O[C@H](CO)[C@H](O)[C@H](O)[C@H]1NC(C)=O)(COCCC(=O)NCCOCCOCCOCCS[C@H]1O[C@H](CO)[C@H](O)[C@H](O)[C@H]1NC(C)=O)NC(=O)CCOCCNC(=O)c1ccc(N2C(=O)C=CC2=O)cc1] |
| 2383 | 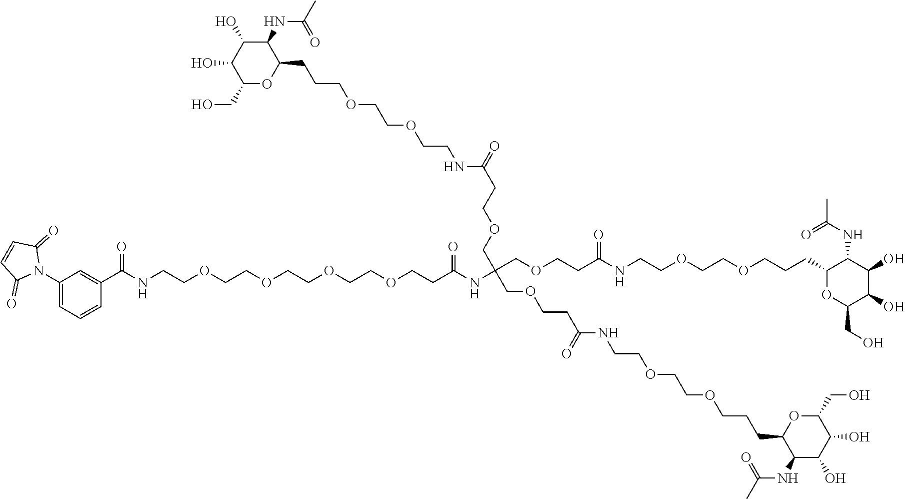 [SMILES: CC(=O)N[C@@H]1[C@@H](O)[C@@H](O)[C@@H](CO)O[C@@H]1CCCOCCOCCNC(=O)CCOCC(COCCC(=O)NCCOCCOCCC[C@H]1O[C@H](CO)[C@H](O)[C@H](O)[C@H]1NC(C)=O)(COCCC(=O)NCCOCCOCCC[C@H]1O[C@H](CO)[C@H](O)[C@H](O)[C@H]1NC(C)=O)NC(=O)CCOCCOCCOCCOCCNC(=O)c1cccc(N2C(=O)C=CC2=O)c1] |
| 2384 | 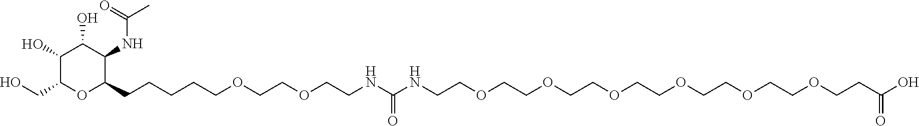 [SMILES: CC(=O)N[C@@H]1[C@@H](O)[C@@H](O)[C@@H](CO)O[C@@H]1CCCCCOCCOCCNC(=O)NCCOCCOCCOCCOCCOCCOCCC(=O)O] |
| 2385 | 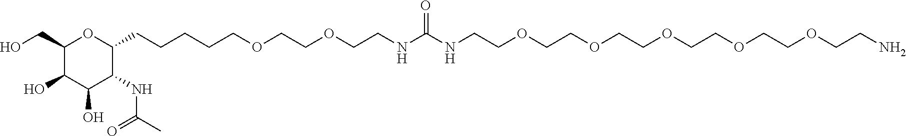 [SMILES: CC(=O)N[C@@H]1[C@@H](O)[C@@H](O)[C@@H](CO)O[C@@H]1CCCCCOCCOCCNC(=O)NCCOCCOCCOCCOCCOCCN] |
| 2386 | 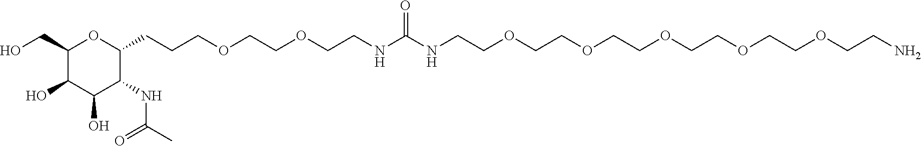 [SMILES: CC(=O)N[C@@H]1[C@@H](O)[C@@H](O)[C@@H](CO)O[C@@H]1CCCOCCOCCNC(=O)NCCOCCOCCOCCOCCOCCN] |
| 2401- C | 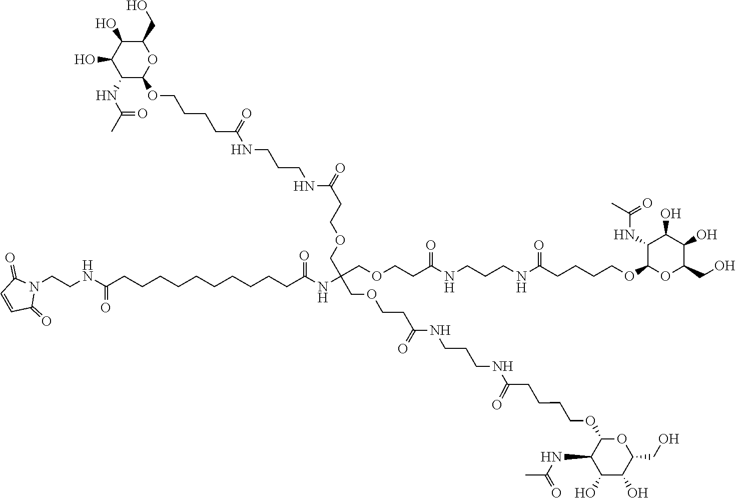 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O] |
| 2402- C | 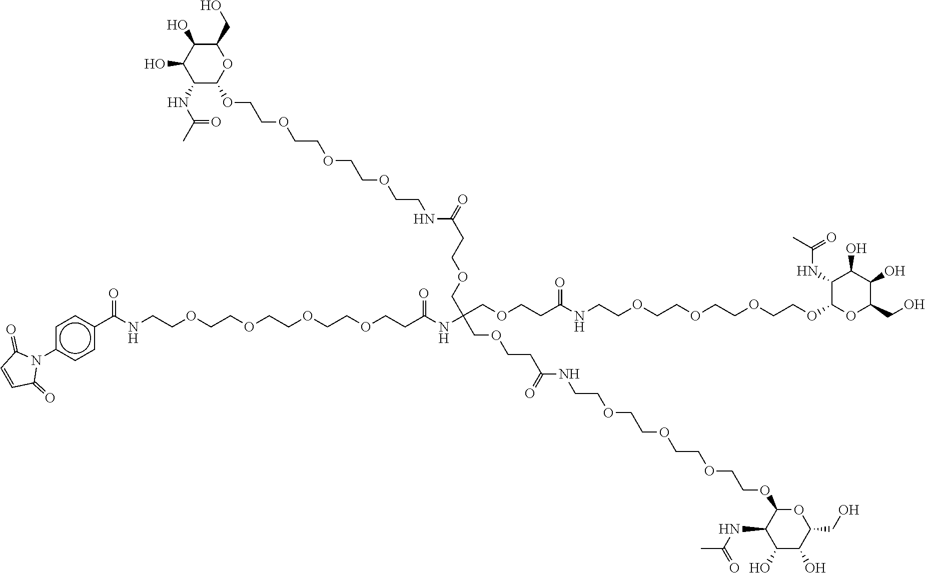 |
| 2403 | 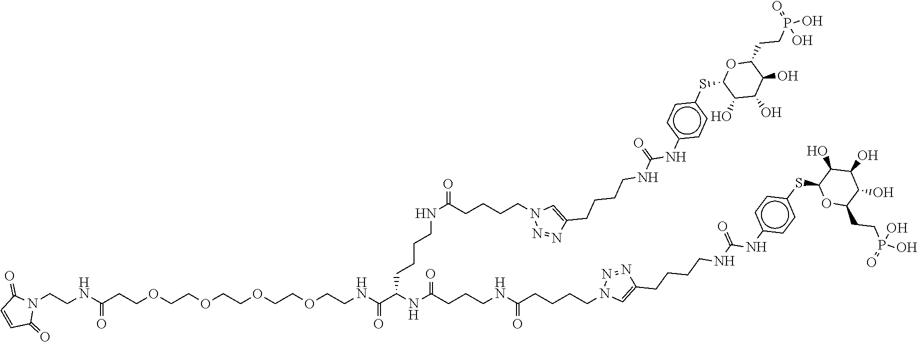 |

Exemplary CI-M6PR-Binding Compounds with Chemoselective Ligation Group for Preparing Conjugates

Exemplary M6PR binding moieties, X, of formula (I)-(XIII) which can be utilized in the preparation of compounds and conjugates of this disclosure are shown in Table 22.

TABLE 22
Exemplary M6PR binding moieties, X

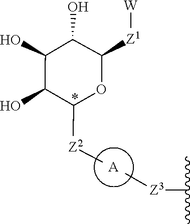

| # | W | Z¹ | Z² | * | A | Z³ |
|---|---|---|---|---|---|---|
| X1 | —P(═O)(OH)₂ | —CH₂CH₂— | —O— | alpha | 1,4-phenylene | —NHCO— |
| X2 | —P(═O)(OH)₂ | —CH₂CH₂— | —O— | alpha | 1,4-phenylene | —NHC(═S)NH— |
| X3 | —P(═O)(OH)₂ | —CH₂CH₂— | —O— | alpha | 1,4-phenylene | —NHC(═O)NH— |
| X4 | —P(═O)(OH)₂ | —CH₂CH₂— | —O— | alpha | 1,4-phenylene | —CH₂— |
| X5 | —P(═O)(OH)₂ | —CH₂CH₂— | —O— | alpha | 1,4-phenylene | —OCH₂— |
| X6 | —P(═O)(OH)₂ | —CH₂CH₂— | —O— | alpha | 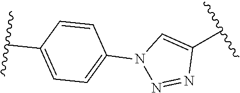 [SMILES: *c1ccc(-n2cc(*)nn2)cc1] | — |
| X7 | —P(═O)(OH)₂ | —CH₂CH₂— | —O— | alpha | 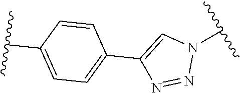 [SMILES: *c1ccc(-c2cn(*)nn2)cc1] | — |
| X8 | —P(═O)(OH)₂ | —CH₂CH₂— | —O— | alpha | 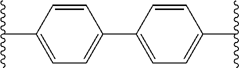 [SMILES: *c1ccc(-c2ccc(*)cc2)cc1] | — |
| X9 | —P(═O)(OH)₂ | —CH₂CH₂— | —O— | alpha | 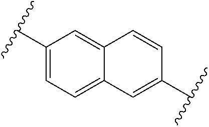 [SMILES: *c1ccc2cc(*)ccc2c1] | —NHCO— |
| X10 | —P(═O)(OH)₂ | —CH₂CH₂— | —O— | alpha | 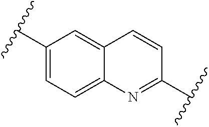 [SMILES: *c1ccc2nc(*)ccc2c1] | —NHCO— |
| X11 | —P(═O)(OH)₂ | —CH₂CH₂— | —S— | alpha | 1,4-phenylene | —NHC(═O)NH— |
| X12 | —P(═O)(OH)₂ | —CH₂CH₂— | —O— | alpha | 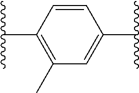 [SMILES: *c1ccc(*)c(C)c1] | —NHC(═O)NH— |
| X13 | —P(═O)(OH)₂ | —CH₂CH₂— | —O— | alpha | 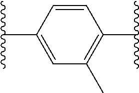 [SMILES: *c1ccc(*)c(C)c1] | —NHC(═O)NH— |
| X14 | —P(═O)(OH)₂ | —CH₂CH₂— | —O— | alpha | 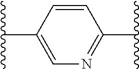 [SMILES: *c1ccc(*)nc1] | —NHC(═O)NH— |
| X15 | —P(═O)(OH)₂ | —CH₂CH₂— | —O— | alpha | 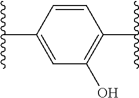 [SMILES: *c1ccc(*)c(O)c1] | —NHC(═O)NH— |
| X16 | —P(═O)(OH)₂ | —CH₂CH₂— | —O— | alpha | 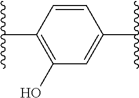 [SMILES: *c1ccc(*)c(O)c1] | —NHC(═O)NH— |
| X17 | —P(═O)(OH)₂ | —CH₂CF₂— | —O— | alpha | 1,4-phenylene | —NHCO— |
| X18 | —P(═O)(OH)₂ | —CH₂CF₂— | —O— | alpha | 1,4-phenylene | —NHC(═S)NH— |
| X19 | —COOH | —CH₂CH₂— | —O— | alpha | 1,4-phenylene | —NHC(═S)NH— |
| X20 | —COOH | —CH═CH— | —O— | alpha | 1,4-phenylene | —NHC(═O)NH— |
| X21 | —CH(COOH)₂ | —CH₂— | —O— | alpha | 1,4-phenylene | —NHCO— |
| X22 | —CH(COOH)₂ | —CH₂— | —O— | alpha | 1,4-phenylene | —NHC(═S)NH— |
| X23 | —CH(COOH)₂ | —CH₂— | —O— | alpha | 1,4-phenylene | —NHC(═O)NH— |
| X24 | —SO₃H | —CH₂CH₂— | —O— | alpha | 1,4-phenylene | —NHCO— |
| X25 | —SO₃H | —CH₂CH₂— | —O— | alpha | 1,4-phenylene | —NHC(═S)NH— |
| X26 | —P(═O)(OH)₂ | —CH₂CH₂— | —CH₂— | alpha | 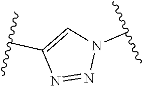 [SMILES: *c1cn(*)nn1] | — |
| X27 | —P(═O)(OH)₂ | —CH₂— | —CH₂— | alpha | 1,4-phenylene | —NHC(═O)NH— |
| X28 | —NHC(═O)CO₂H | —CH₂— | —O— | alpha | 1,4-phenylene | —NHC(═O)NH— |
| X29 | 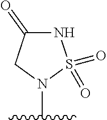 [SMILES: *N1CC(=O)NS1(=O)=O] | —CH₂— | —O— | alpha | 1,4-phenylene | —NHC(═O)NH— |
| X30 | —NHSO₂Me | —CH₂— | —O— | alpha | 1,4-phenylene | —NHC(═O)NH— |
| X31 | —NHSO₂NH₂ | —CH₂— | —O— | alpha | 1,4-phenylene | —NHC(═O)NH— |
| X32 | —NHC(═O)NHSO₂Me | —CH₂— | —O— | alpha | 1,4-phenylene | —NHC(═O)NH— |
| X33 | —NHSO₃H | —CH₂— | —O— | alpha | 1,4-phenylene | —NHC(═O)NH— |
| X34 | —P(═O)(OH)₂ | —CH₂CH₂— | —O— | alpha | 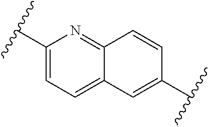 [SMILES: *c1ccc2nc(*)ccc2c1] | —NHC(═O)NH— |
| X35 | —P(═O)(OH)₂ | —CH₂CH₂— | —O— | alpha | 1,4-phenylene | —CONH— |
| X36 | —P(═O)(OH)₂ | —CH₂CH₂— | —O— | alpha | 1,4-phenylene | —NHSO₂— |
| X37 | —P(═O)(OH)₂ | —CH₂CH₂— | —O— | alpha | 1,4-phenylene | —SO₂NH— |
| X38 | —NHSO₂CF₃ | —CH₂— | —O— | alpha | 1,4-phenylene | —NHC(═O)NH— |
| X39 | —P(═O)(OH)₂ | —CH₂CH₂— | —CF₂— | alpha | 1,4-phenylene | —NHC(═O)NH— |
| X40 | —P(═O)(OH)₂ | —CH₂CH₂— | —CF₂— | alpha | 1,4-phenylene | —NHC(═S)NH— |
| X41 | —P(═O)(OH)₂ | —CH₂CH₂— | —CH₂— | alpha | 1,4-phenylene | —NHC(═S)NH— |
| X42 | —P(═O)(OH)₂ | —CH₂CH₂— | —S— | alpha | 1,4-phenylene | —NHC(═S)NH— |
| X43 | —P(═O)(OH)₂ | —CH₂CH₂— | —S— | alpha | 1,4-phenylene | —NHCO— |
| X44 | —P(═O)(OH)₂ | —CH₂CH₂— | —S— | alpha | 1,4-phenylene | —CONH— |
| X45 | —P(═O)(OH)₂ | —CH₂CH₂— | —S— | alpha | 1,4-phenylene | —NHSO₂— |
| X46 | —P(═O)(OH)₂ | —CH₂CH₂— | —S— | alpha | 1,4-phenylene | —SO₂NH— |
| X47 | —P(═O)(OH)₂ | —CH₂CH₂— | —S— | alpha | 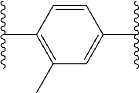 [SMILES: *c1ccc(*)c(C)c1] | —NHC(═O)NH— |
| X48 | —P(═O)(OH)₂ | —CH₂CH₂— | —S— | alpha | 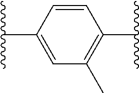 [SMILES: *c1ccc(*)c(C)c1] | —NHC(═O)NH— |
| X49 | —P(═O)(OH)₂ | —CH₂CH₂— | —S— | alpha | 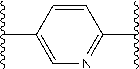 [SMILES: *c1ccc(*)nc1] | —NHC(═O)NH— |
| X50 | —P(═O)(OH)₂ | —CH₂CH₂— | —S— | alpha | 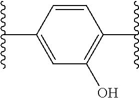 [SMILES: *c1ccc(*)c(O)c1] | —NHC(═O)NH— |
| X51 | —P(═O)(OH)₂ | —CH₂CH₂— | —S— | alpha | 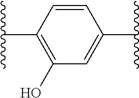 [SMILES: *c1ccc(*)c(O)c1] | —NHC(═O)NH— |

Beta configuration moieties

| | | | | | | |
|---|---|---|---|---|---|---|
| X1* | —P(═O)(OH)₂ | —CH₂CH₂— | —O— | beta | 1,4-phenylene | —NHCO— |
| X2* | —P(═O)(OH)₂ | —CH₂CH₂— | —O— | beta | 1,4-phenylene | —NHC(═S)NH— |
| X3* | —P(═O)(OH)₂ | —CH₂CH₂— | —O— | beta | 1,4-phenylene | —NHC(═O)NH— |
| X4* | —P(═O)(OH)₂ | —CH₂CH₂— | —O— | beta | 1,4-phenylene | —CH₂— |
| X5* | —P(═O)(OH)₂ | —CH₂CH₂— | —O— | beta | 1,4-phenylene | —OCH₂— |
| X6* | —P(═O)(OH)₂ | —CH₂CH₂— | —O— | beta | 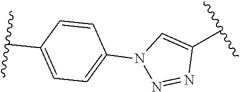 [SMILES: *c1ccc(-n2cc(*)nn2)cc1] | — |
| X7* | —P(═O)(OH)₂ | —CH₂CH₂— | —O— | beta | 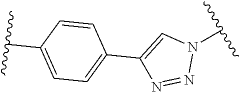 [SMILES: *c1ccc(-c2cn(*)nn2)cc1] | — |
| X8* | —P(═O)(OH)₂ | —CH₂CH₂— | —O— | beta | 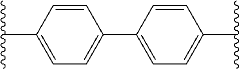 [SMILES: *c1ccc(-c2ccc(*)cc2)cc1] | — |
| X9* | —P(═O)(OH)₂ | —CH₂CH₂— | —O— | beta | 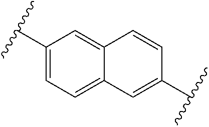 [SMILES: *c1ccc2cc(*)ccc2c1] | —NHCO— |
| X10* | —P(═O)(OH)₂ | —CH₂CH₂— | —O— | beta | 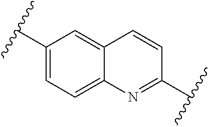 [SMILES: *c1ccc2nc(*)ccc2c1] | —NHCO— |
| X11* | —P(═O)(OH)₂ | —CH₂CH₂— | —S— | beta | 1,4-phenylene | —NHC(═O)NH— |
| X12* | —P(═O)(OH)₂ | —CH₂CH₂— | —O— | beta | 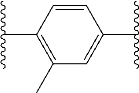 [SMILES: *c1ccc(*)c(C)c1] | —NHC(═O)NH— |
| X13* | —P(═O)(OH)₂ | —CH₂CH₂— | —O— | beta | 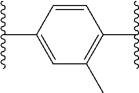 [SMILES: *c1ccc(*)c(C)c1] | —NHC(═O)NH— |
| X14* | —P(═O)(OH)₂ | —CH₂CH₂— | —O— | beta | 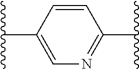 [SMILES: *c1ccc(*)nc1] | —NHC(═O)NH— |
| X15* | —P(═O)(OH)₂ | —CH₂CH₂— | —O— | beta | 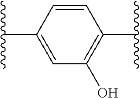 [SMILES: *c1ccc(*)c(O)c1] | —NHC(═O)NH— |
| X16* | —P(═O)(OH)₂ | —CH₂CH₂— | —O— | beta | 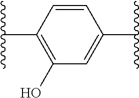 [SMILES: *c1ccc(*)c(O)c1] | —NHC(═O)NH— |
| X17* | —P(═O)(OH)₂ | —CH₂CF₂— | —O— | beta | 1,4-phenylene | —NHCO— |
| X18* | —P(═O)(OH)₂ | —CH₂CF₂ | —O— | beta | 1,4-phenylene | —NHC(═S)NH— |
| X19* | —COOH | —CH₂CH₂— | —O— | beta | 1,4-phenylene | —NHC(═S)NH— |
| X20* | —COOH | —CH═CH— | —O— | beta | 1,4-phenylene | —NHC(═O)NH— |
| X21* | —CH(COOH)₂ | —CH₂— | —O— | beta | 1,4-phenylene | —NHCO— |
| X22* | —CH(COOH)₂ | —CH₂— | —O— | beta | 1,4-phenylene | —NHC(═S)NH— |
| X23* | —CH(COOH)₂ | —CH₂— | —O— | beta | 1,4-phenylene | —NHC(═O)NH— |
| X24* | —SO₃H | —CH₂CH₂— | —O— | beta | 1,4-phenylene | —NHCO— |
| X25* | —SO₃H | —CH₂CH₂— | —O— | beta | 1,4-phenylene | —NHC(═S)NH— |
| X26* | —P(═O)(OH)₂ | —CH₂CH₂— | —CH₂— | beta | 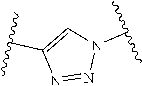 [SMILES: *c1cn(*)nn1] | — |
| X27* | —P(═O)(OH)₂ | —CH₂CH₂— | —CH₂— | beta | 1,4-phenylene | —NHC(═O)NH— |
| X28* | —NHC(═O)CO₂H | —CH₂CH₂— | —O— | beta | 1,4-phenylene | —NHC(═O)NH— |
| X29* | 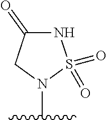 [SMILES: *N1CC(=O)NS1(=O)=O] | —CH₂— | —O— | beta | 1,4-phenylene | —NHC(═O)NH— |
| X30* | —NHSO₂Me | —CH₂— | —O— | beta | 1,4-phenylene | —NHC(═O)NH— |
| X31* | —NHSO₂NH₂ | —CH₂— | —O— | beta | 1,4-phenylene | —NHC(═O)NH— |
| X32* | —NHC(═O)NHSO₂Me | —CH₂— | —O— | beta | 1,4-phenylene | —NHC(═O)NH— |
| X33* | —NHSO₃H | —CH₂— | —O— | beta | 1,4-phenylene | —NHC(═O)NH— |
| X34* | —P(═O)(OH)₂ | —CH₂CH₂— | —O— | beta | 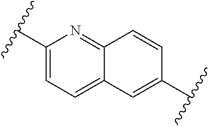 [SMILES: *c1ccc2nc(*)ccc2c1] | —NHC(═O)NH— |
| X35* | —P(═O)(OH)₂ | —CH₂CH₂— | —O— | beta | 1,4-phenylene | —CONH— |
| X36* | —P(═O)(OH)₂ | —CH₂CH₂— | —O— | beta | 1,4-phenylene | —NHSO₂— |
| X37* | —P(═O)(OH)₂ | —CH₂CH₂— | —O— | beta | 1,4-phenylene | —SO₂NH— |
| X38* | —NHSO₂CF₃— | —CH₂— | —O— | beta | 1,4-phenylene | —NHC(═O)NH— |
| X39* | —P(═O)(OH)₂ | —CH₂CH₂— | —CF₂— | beta | 1,4-phenylene | —NHC(═O)NH— |
| X40* | —P(═O)(OH)₂ | —CH₂CH₂— | —CF₂— | beta | 1,4-phenylene | —NHC(═S)NH— |
| X41* | —P(═O)(OH)₂ | —CH₂CH₂— | —CH₂— | beta | 1,4-phenylene | —NHC(═S)NH— |
| X42* | —P(═O)(OH)₂ | —CH₂CH₂— | —S— | beta | 1,4-phenylene | —NHC(═S)NH— |
| X43* | —P(═O)(OH)₂ | —CH₂CH₂— | —S— | beta | 1,4-phenylene | —NHCO— |
| X44* | —P(═O)(OH)₂ | —CH₂CH₂— | —S— | beta | 1,4-phenylene | —CONH— |
| X45* | —P(═O)(OH)₂ | —CH₂CH₂— | —S— | beta | 1,4-phenylene | —NHSO₂— |
| X46* | —P(═O)(OH)₂ | —CH₂CH₂— | —S— | beta | 1,4-phenylene | —SO₂NH— |
| X47* | —P(═O)(OH)₂ | —CH₂CH₂— | —S— | beta | 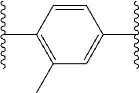 [SMILES: *c1ccc(*)c(C)c1] | —NHC(═O)NH— |
| X48* | —P(═O)(OH)₂ | —CH₂CH₂— | —S— | beta | 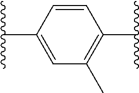 [SMILES: *c1ccc(*)c(C)c1] | —NHC(═O)NH— |
| X49* | —P(═O)(OH)₂ | —CH₂CH₂— | —S— | beta | 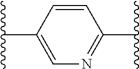 [SMILES: *c1ccc(*)nc1] | —NHC(═O)NH— |
| X50* | —P(═O)(OH)₂ | —CH₂CH₂— | —S— | beta | 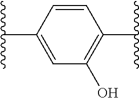 [SMILES: *c1ccc(*)c(O)c1] | —NHC(═O)NH— |
| X51* | —P(═O)(OH)₂ | —CH₂CH₂— | —S— | beta | 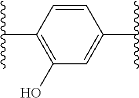 [SMILES: *c1ccc(*)c(O)c1] | —NHC(═O)NH— |

alpha refers to the following configuration: 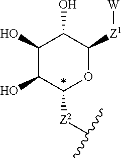
*beta refers to the following configuration:

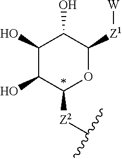

Exemplary synthons or synthetic precursors which can be utilized in the preparation of compounds of this disclosure to incorporate a desired M6PR binding moiety of interest are shown in Table 23. It is understood that alternative synthons, including homologs and analogs of the ones shown in Table 23 are possible depending on the M6PR binding moiety and linker that is selected. It is understood that the synthons of Table 23 can include structural precursors of linking moiety Z³, and a structural element that becomes part of the linker (L) in the compounds and conjugates of this disclosure. It is understood that based on the exemplary synthetic precursors of Table 23, synthons corresponding to any of the M6PR binding moieties of Table 22 can be utilized to prepare compounds of this disclosure.

TABLE 23
Exemplary Synthetic precursors for M6PR binding moieties

| Exemplary M6PR binding moiety (X) | | Exemplary Synthetic precursor(s) |
|---|---|---|
| # | Structure | Structure |
| X1 | 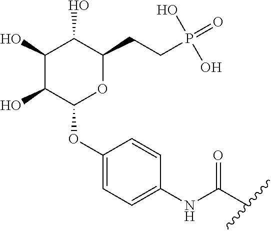 [SMILES: *C(=O)Nc1ccc(O[C@H]2O[C@H](CCP(=O)(O)O)[C@@H](O)[C@H](O)[C@@H]2O)cc1] | 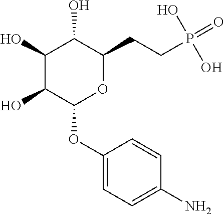 [SMILES: Nc1ccc(O[C@H]2O[C@H](CCP(=O)(O)O)[C@@H](O)[C@H](O)[C@@H]2O)cc1] |
| | | 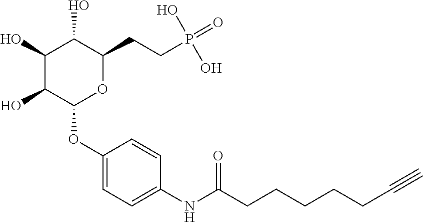 [SMILES: C#CCCCCCC(=O)Nc1ccc(O[C@H]2O[C@H](CCP(=O)(O)O)[C@@H](O)[C@H](O)[C@@H]2O)cc1] |
| X2 | 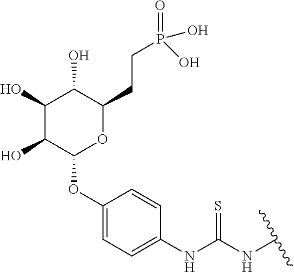 [SMILES: *NC(=S)Nc1ccc(O[C@H]2O[C@H](CCP(=O)(O)O)[C@@H](O)[C@H](O)[C@@H]2O)cc1] | 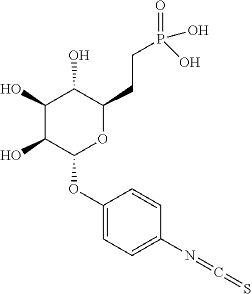 [SMILES: O=P(O)(O)CC[C@H]1O[C@H](Oc2ccc(N=C=S)cc2)[C@@H](O)[C@@H](O)[C@@H]1O] |
| | | 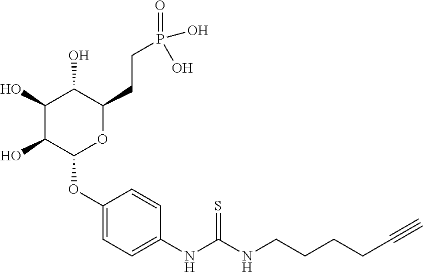 [SMILES: C#CCCCCNC(=S)Nc1ccc(O[C@H]2O[C@H](CCP(=O)(O)O)[C@@H](O)[C@H](O)[C@@H]2O)cc1] |
| X3 | 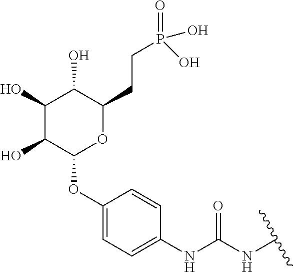 [SMILES: *NC(=O)Nc1ccc(O[C@H]2O[C@H](CCP(=O)(O)O)[C@@H](O)[C@H](O)[C@@H]2O)cc1] | 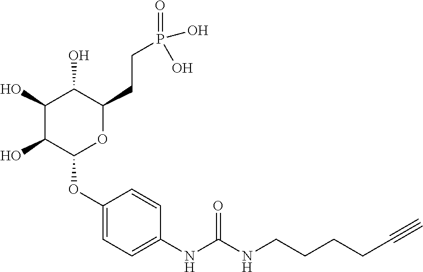 [SMILES: C#CCCCCNC(=O)Nc1ccc(O[C@H]2O[C@H](CCP(=O)(O)O)[C@@H](O)[C@H](O)[C@@H]2O)cc1] |
| X4 | 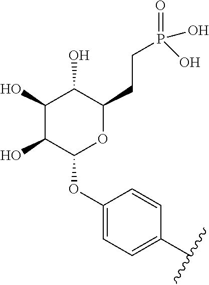 [SMILES: *c1ccc(O[C@H]2O[C@H](CCP(=O)(O)O)[C@@H](O)[C@H](O)[C@@H]2O)cc1] | 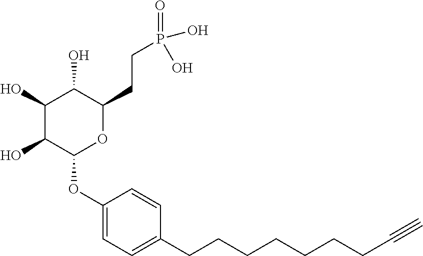 [SMILES: C#CCCCCCCCc1ccc(O[C@H]2O[C@H](CCP(=O)(O)O)[C@@H](O)[C@H](O)[C@@H]2O)cc1] |
| | | 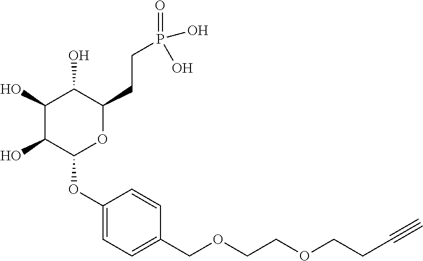 [SMILES: C#CCCOCCOCc1ccc(O[C@H]2O[C@H](CCP(=O)(O)O)[C@@H](O)[C@H](O)[C@@H]2O)cc1] |
| X5 | 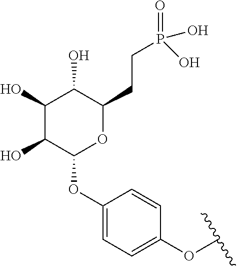 [SMILES: *Oc1ccc(O[C@H]2O[C@H](CCP(=O)(O)O)[C@@H](O)[C@H](O)[C@@H]2O)cc1] | 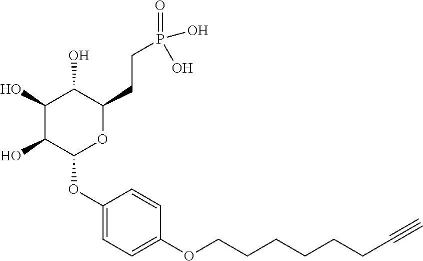 [SMILES: C#CCCCCCCOc1ccc(O[C@H]2O[C@H](CCP(=O)(O)O)[C@@H](O)[C@H](O)[C@@H]2O)cc1] |
| X6 | 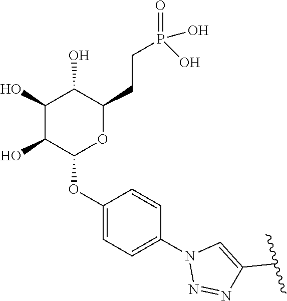 [SMILES: *c1cn(-c2ccc(O[C@H]3O[C@H](CCP(=O)(O)O)[C@@H](O)[C@H](O)[C@@H]3O)cc2)nn1] | 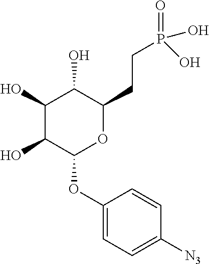 [SMILES: [N-]=[N+]=Nc1ccc(O[C@H]2O[C@H](CCP(=O)(O)O)[C@@H](O)[C@H](O)[C@@H]2O)cc1] |
| X7 | 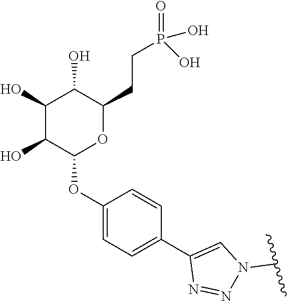 [SMILES: *n1cc(-c2ccc(O[C@H]3O[C@H](CCP(=O)(O)O)[C@@H](O)[C@H](O)[C@@H]3O)cc2)nn1] | 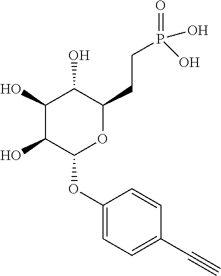 [SMILES: C#Cc1ccc(O[C@H]2O[C@H](CCP(=O)(O)O)[C@@H](O)[C@H](O)[C@@H]2O)cc1] |
| X8 | 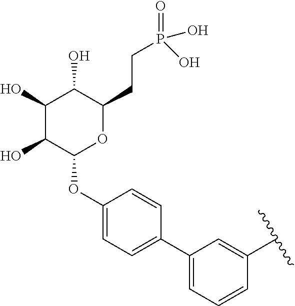 [SMILES: *c1cccc(-c2ccc(O[C@H]3O[C@H](CCP(=O)(O)O)[C@@H](O)[C@H](O)[C@@H]3O)cc2)c1] | 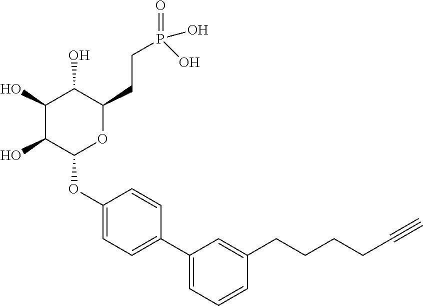 [SMILES: C#CCCCCc1cccc(-c2ccc(O[C@H]3O[C@H](CCP(=O)(O)O)[C@@H](O)[C@H](O)[C@@H]3O)cc2)c1] |
| X9 | 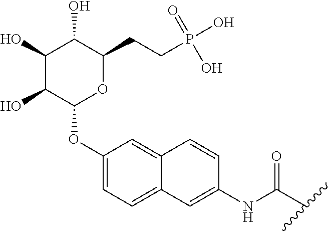 [SMILES: *C(=O)Nc1ccc2cc(O[C@H]3O[C@H](CCP(=O)(O)O)[C@@H](O)[C@H](O)[C@@H]3O)ccc2c1] | 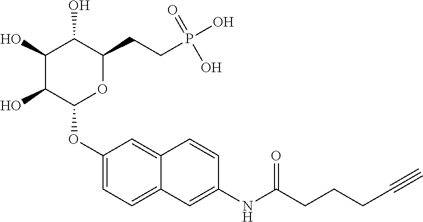 [SMILES: C#CCCCC(=O)Nc1ccc2cc(O[C@H]3O[C@H](CCP(=O)(O)O)[C@@H](O)[C@H](O)[C@@H]3O)ccc2c1] |
| X10 | 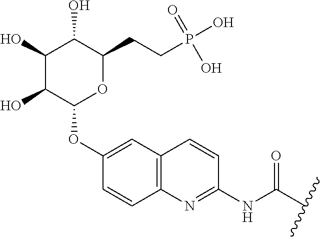 [SMILES: *C(=O)Nc1ccc2cc(O[C@H]3O[C@H](CCP(=O)(O)O)[C@@H](O)[C@H](O)[C@@H]3O)ccc2n1] | 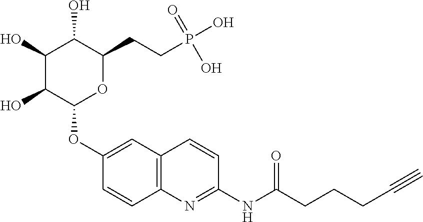 [SMILES: C#CCCCC(=O)Nc1ccc2cc(O[C@H]3O[C@H](CCP(=O)(O)O)[C@@H](O)[C@H](O)[C@@H]3O)ccc2n1] |
| X11 | 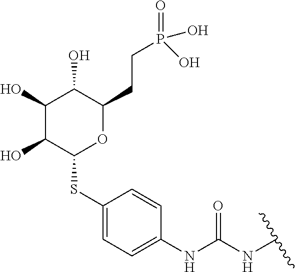 [SMILES: *NC(=O)Nc1ccc(S[C@H]2O[C@H](CCP(=O)(O)O)[C@@H](O)[C@H](O)[C@@H]2O)cc1] | 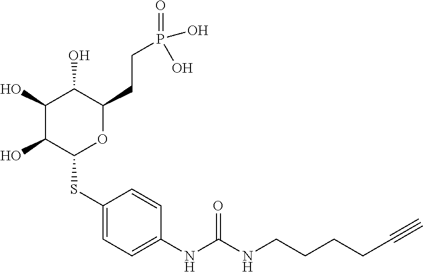 [SMILES: C#CCCCCNC(=O)Nc1ccc(S[C@H]2O[C@H](CCP(=O)(O)O)[C@@H](O)[C@H](O)[C@@H]2O)cc1] |
| X11* | 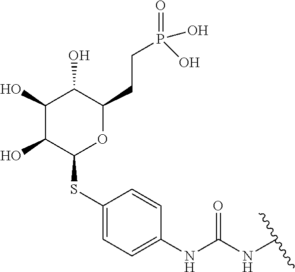 [SMILES: *NC(=O)Nc1ccc(S[C@@H]2O[C@H](CCP(=O)(O)O)[C@@H](O)[C@H](O)[C@@H]2O)cc1] | 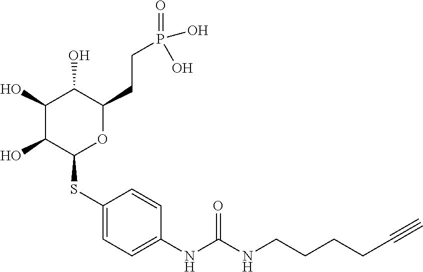 [SMILES: C#CCCCCNC(=O)Nc1ccc(S[C@@H]2O[C@H](CCP(=O)(O)O)[C@@H](O)[C@H](O)[C@@H]2O)cc1] |
| X12 | 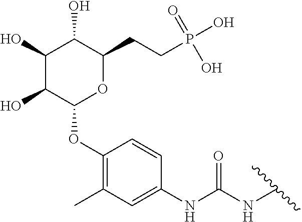 [SMILES: *NC(=O)Nc1ccc(O[C@H]2O[C@H](CCP(=O)(O)O)[C@@H](O)[C@H](O)[C@@H]2O)c(C)c1] | 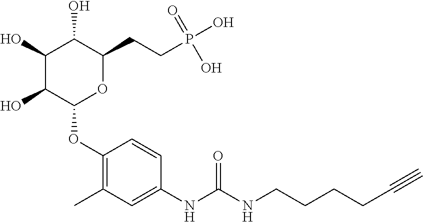 [SMILES: C#CCCCCNC(=O)Nc1ccc(O[C@H]2O[C@H](CCP(=O)(O)O)[C@@H](O)[C@H](O)[C@@H]2O)c(C)c1] |
| X13 | 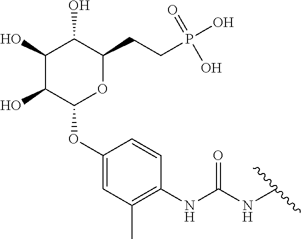 [SMILES: *NC(=O)Nc1ccc(O[C@H]2O[C@H](CCP(=O)(O)O)[C@@H](O)[C@H](O)[C@@H]2O)cc1C] | 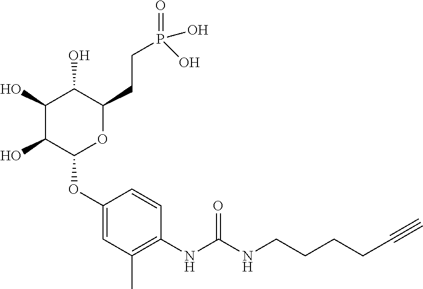 [SMILES: C#CCCCCNC(=O)Nc1ccc(O[C@H]2O[C@H](CCP(=O)(O)O)[C@@H](O)[C@H](O)[C@@H]2O)cc1C] |
| X14 | 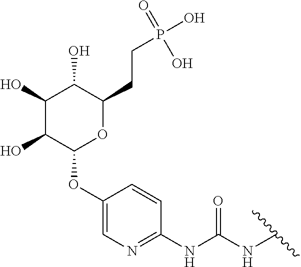 [SMILES: *NC(=O)Nc1ccc(O[C@H]2O[C@H](CCP(=O)(O)O)[C@@H](O)[C@H](O)[C@@H]2O)cn1] | 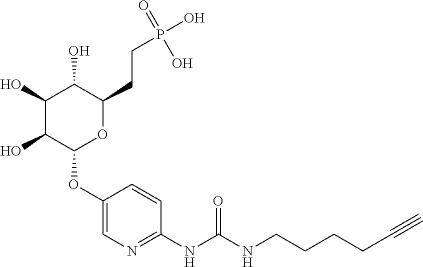 [SMILES: C#CCCCCNC(=O)Nc1ccc(O[C@H]2O[C@H](CCP(=O)(O)O)[C@@H](O)[C@H](O)[C@@H]2O)cn1] |
| X15 | 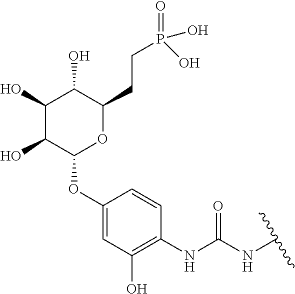 [SMILES: *NC(=O)Nc1ccc(O[C@H]2O[C@H](CCP(=O)(O)O)[C@@H](O)[C@H](O)[C@@H]2O)cc1O] | 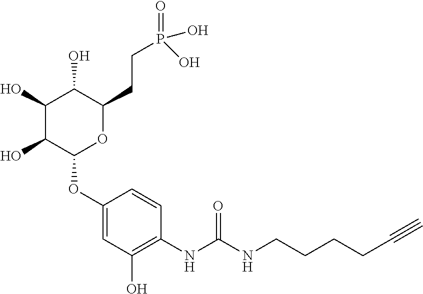 [SMILES: C#CCCCCNC(=O)Nc1ccc(O[C@H]2O[C@H](CCP(=O)(O)O)[C@@H](O)[C@H](O)[C@@H]2O)cc1O] |
| X16 | 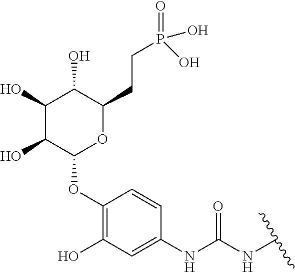 [SMILES: *NC(=O)Nc1ccc(O[C@H]2O[C@H](CCP(=O)(O)O)[C@@H](O)[C@H](O)[C@@H]2O)c(O)c1] | 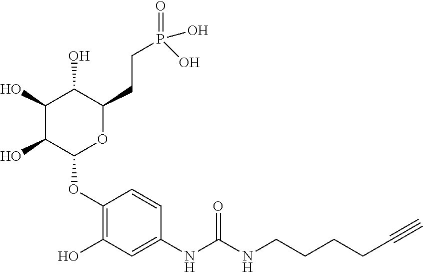 [SMILES: C#CCCCCNC(=O)Nc1ccc(O[C@H]2O[C@H](CCP(=O)(O)O)[C@@H](O)[C@H](O)[C@@H]2O)c(O)c1] |
| X17 | 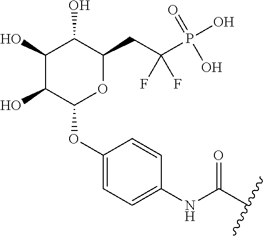 [SMILES: *C(=O)Nc1ccc(O[C@H]2O[C@H](CC(F)(F)P(=O)(O)O)[C@@H](O)[C@H](O)[C@@H]2O)cc1] | 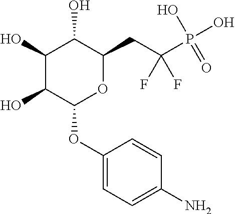 [SMILES: Nc1ccc(O[C@H]2O[C@H](CC(F)(F)P(=O)(O)O)[C@@H](O)[C@H](O)[C@@H]2O)cc1] |
| X18 | 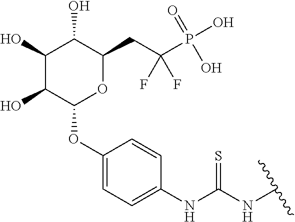 [SMILES: *NC(=S)Nc1ccc(O[C@H]2O[C@H](CC(F)(F)P(=O)(O)O)[C@@H](O)[C@H](O)[C@@H]2O)cc1] | 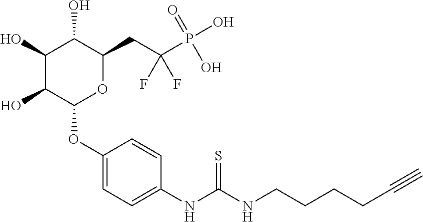 [SMILES: C#CCCCCNC(=S)Nc1ccc(O[C@H]2O[C@H](CC(F)(F)P(=O)(O)O)[C@@H](O)[C@H](O)[C@@H]2O)cc1] |
| X19 | 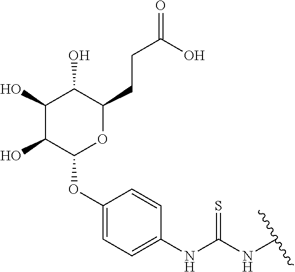 [SMILES: *NC(=S)Nc1ccc(O[C@H]2O[C@H](CCC(=O)O)[C@@H](O)[C@H](O)[C@@H]2O)cc1] | 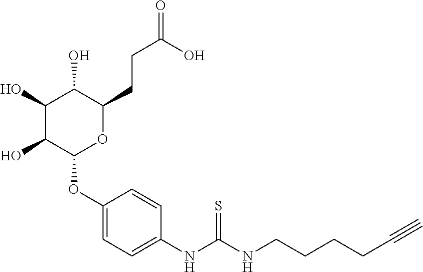 [SMILES: C#CCCCCNC(=S)Nc1ccc(O[C@H]2O[C@H](CCC(=O)O)[C@@H](O)[C@H](O)[C@@H]2O)cc1] |
| X20 | 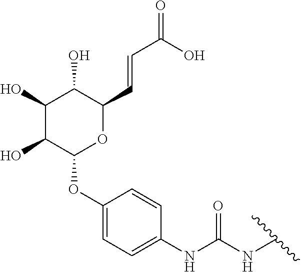 [SMILES: *NC(=O)Nc1ccc(O[C@H]2O[C@H](/C=C/C(=O)O)[C@@H](O)[C@H](O)[C@@H]2O)cc1] | 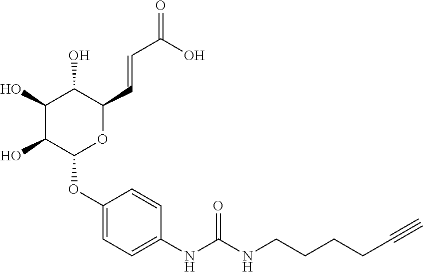 [SMILES: C#CCCCCNC(=O)Nc1ccc(O[C@H]2O[C@H](/C=C/C(=O)O)[C@@H](O)[C@H](O)[C@@H]2O)cc1] |
| X21 | 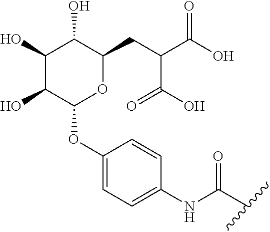 [SMILES: *C(=O)Nc1ccc(O[C@H]2O[C@H](CC(C(=O)O)C(=O)O)[C@@H](O)[C@H](O)[C@@H]2O)cc1] | 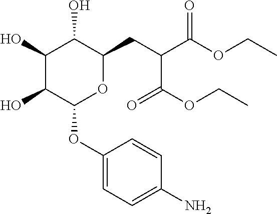 [SMILES: CCOC(=O)C(C[C@H]1O[C@H](Oc2ccc(N)cc2)[C@@H](O)[C@@H](O)[C@@H]1O)C(=O)OCC] |
| X22 | 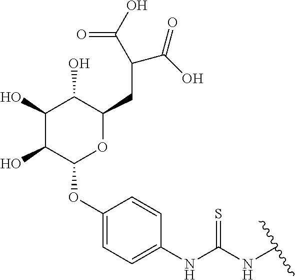 [SMILES: *NC(=S)Nc1ccc(O[C@H]2O[C@H](CC(C(=O)O)C(=O)O)[C@@H](O)[C@H](O)[C@@H]2O)cc1] | 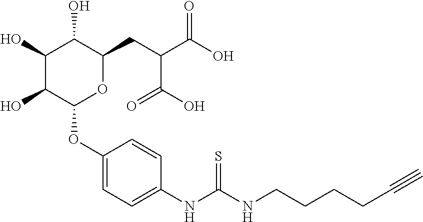 [SMILES: C#CCCCCNC(=S)Nc1ccc(O[C@H]2O[C@H](CC(C(=O)O)C(=O)O)[C@@H](O)[C@H](O)[C@@H]2O)cc1] |
| X23 | 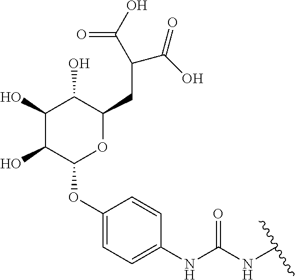 [SMILES: *NC(=O)Nc1ccc(O[C@H]2O[C@H](CC(C(=O)O)C(=O)O)[C@@H](O)[C@H](O)[C@@H]2O)cc1] | 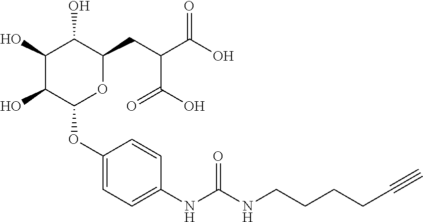 [SMILES: C#CCCCCNC(=O)Nc1ccc(O[C@H]2O[C@H](CC(C(=O)O)C(=O)O)[C@@H](O)[C@H](O)[C@@H]2O)cc1] |
| X24 | 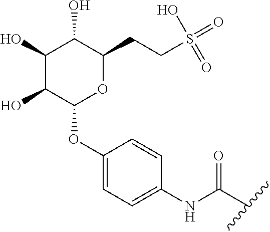 [SMILES: *C(=O)Nc1ccc(O[C@H]2O[C@H](CCS(=O)(=O)O)[C@@H](O)[C@H](O)[C@@H]2O)cc1] | 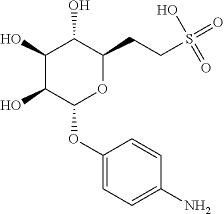 [SMILES: Nc1ccc(O[C@H]2O[C@H](CCS(=O)(=O)O)[C@@H](O)[C@H](O)[C@@H]2O)cc1] |
| X25 | 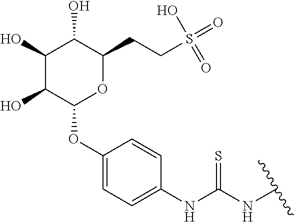 [SMILES: *NC(=S)Nc1ccc(O[C@H]2O[C@H](CCS(=O)(=O)O)[C@@H](O)[C@H](O)[C@@H]2O)cc1] | 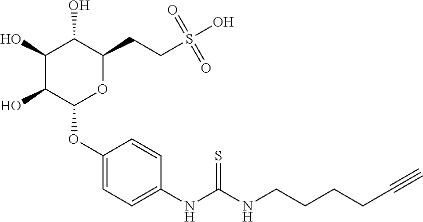 [SMILES: C#CCCCCNC(=S)Nc1ccc(O[C@H]2O[C@H](CCS(=O)(=O)O)[C@@H](O)[C@H](O)[C@@H]2O)cc1] |
| X26 | 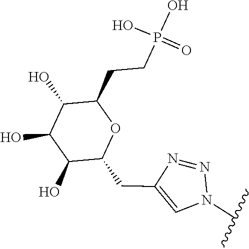 [SMILES: *n1cc(C[C@H]2O[C@H](CCP(=O)(O)O)[C@@H](O)[C@H](O)[C@@H]2O)nn1] | 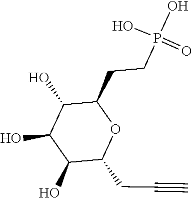 [SMILES: C#CC[C@H]1O[C@H](CCP(=O)(O)O)[C@@H](O)[C@H](O)[C@@H]1O] |
| X27 | 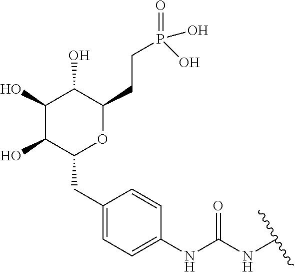 [SMILES: *NC(=O)Nc1ccc(C[C@H]2O[C@H](CCP(=O)(O)O)[C@@H](O)[C@H](O)[C@@H]2O)cc1] | 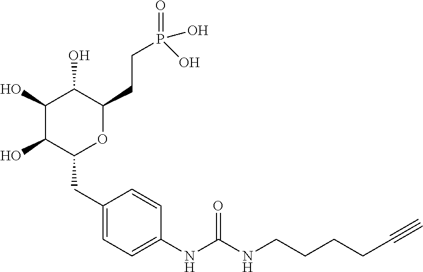 [SMILES: C#CCCCCNC(=O)Nc1ccc(C[C@H]2O[C@H](CCP(=O)(O)O)[C@@H](O)[C@H](O)[C@@H]2O)cc1] |
| X28 | 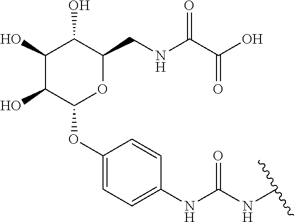 [SMILES: *NC(=O)Nc1ccc(O[C@H]2O[C@H](CNC(=O)C(=O)O)[C@@H](O)[C@H](O)[C@@H]2O)cc1] | 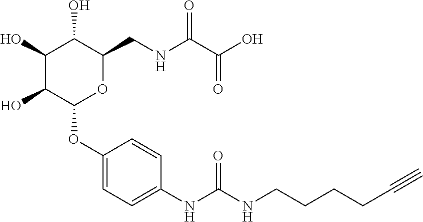 [SMILES: C#CCCCCNC(=O)Nc1ccc(O[C@H]2O[C@H](CNC(=O)C(=O)O)[C@@H](O)[C@H](O)[C@@H]2O)cc1] |
| X29 | 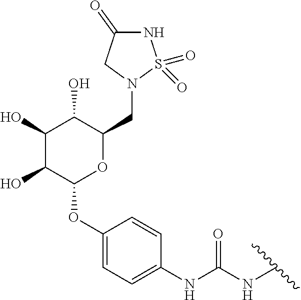 [SMILES: *NC(=O)Nc1ccc(O[C@H]2O[C@H](CN3CC(=O)NS3(=O)=O)[C@@H](O)[C@H](O)[C@@H]2O)cc1] | 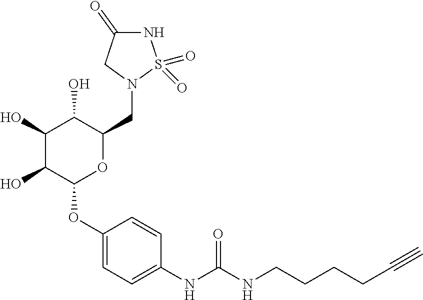 [SMILES: C#CCCCCNC(=O)Nc1ccc(O[C@H]2O[C@H](CN3CC(=O)NS3(=O)=O)[C@@H](O)[C@H](O)[C@@H]2O)cc1] |
| X30 | 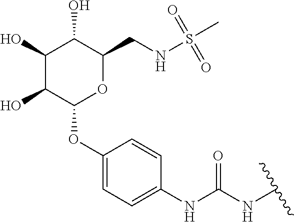 [SMILES: *NC(=O)Nc1ccc(O[C@H]2O[C@H](CNS(C)(=O)=O)[C@@H](O)[C@H](O)[C@@H]2O)cc1] | 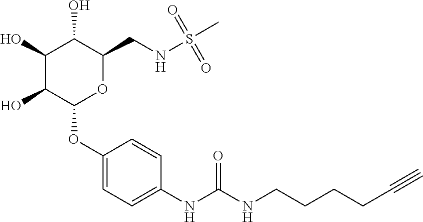 [SMILES: C#CCCCCNC(=O)Nc1ccc(O[C@H]2O[C@H](CNS(C)(=O)=O)[C@@H](O)[C@H](O)[C@@H]2O)cc1] |
| X31 | 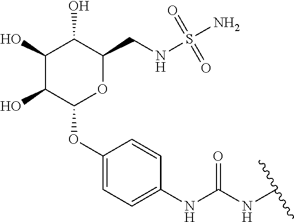 [SMILES: *NC(=O)Nc1ccc(O[C@H]2O[C@H](CNS(N)(=O)=O)[C@@H](O)[C@H](O)[C@@H]2O)cc1] | 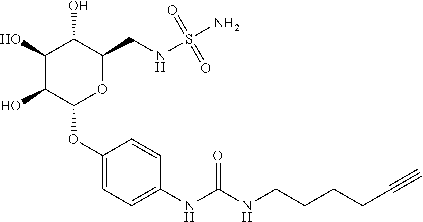 [SMILES: C#CCCCCNC(=O)Nc1ccc(O[C@H]2O[C@H](CNS(N)(=O)=O)[C@@H](O)[C@H](O)[C@@H]2O)cc1] |
| X32 | 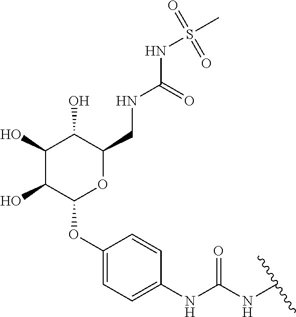 [SMILES: *NC(=O)Nc1ccc(O[C@H]2O[C@H](CNC(=O)NS(C)(=O)=O)[C@@H](O)[C@H](O)[C@@H]2O)cc1] | 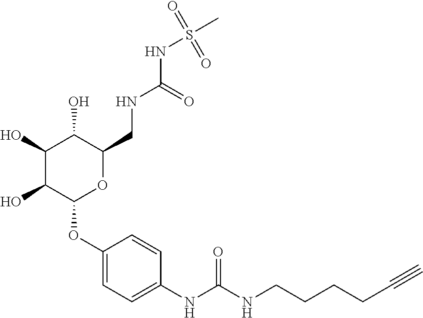 [SMILES: C#CCCCCNC(=O)Nc1ccc(O[C@H]2O[C@H](CNC(=O)NS(C)(=O)=O)[C@@H](O)[C@H](O)[C@@H]2O)cc1] |
| X33 | 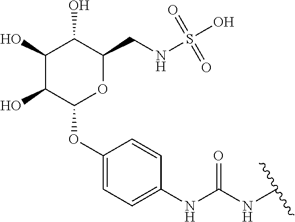 [SMILES: *NC(=O)Nc1ccc(O[C@H]2O[C@H](CNS(=O)(=O)O)[C@@H](O)[C@H](O)[C@@H]2O)cc1] | 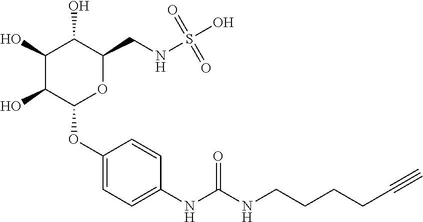 [SMILES: C#CCCCCNC(=O)Nc1ccc(O[C@H]2O[C@H](CNS(=O)(=O)O)[C@@H](O)[C@H](O)[C@@H]2O)cc1] |
| X34 | 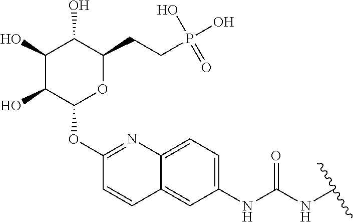 [SMILES: *NC(=O)Nc1ccc2nc(O[C@H]3O[C@H](CCP(=O)(O)O)[C@@H](O)[C@H](O)[C@@H]3O)ccc2c1] | 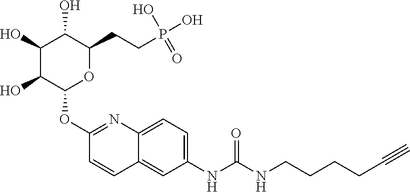 [SMILES: C#CCCCCNC(=O)Nc1ccc2nc(O[C@H]3O[C@H](CCP(=O)(O)O)[C@@H](O)[C@H](O)[C@@H]3O)ccc2c1] |
| X35 | 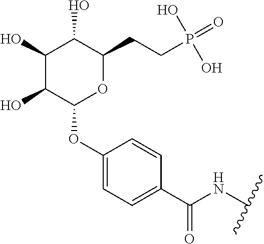 [SMILES: *NC(=O)c1ccc(O[C@H]2O[C@H](CCP(=O)(O)O)[C@@H](O)[C@H](O)[C@@H]2O)cc1] | 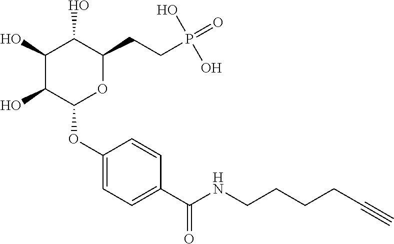 [SMILES: C#CCCCCNC(=O)c1ccc(O[C@H]2O[C@H](CCP(=O)(O)O)[C@@H](O)[C@H](O)[C@@H]2O)cc1] |
| X36 | 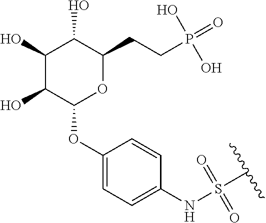 [SMILES: *S(=O)(=O)Nc1ccc(O[C@H]2O[C@H](CCP(=O)(O)O)[C@@H](O)[C@H](O)[C@@H]2O)cc1] | 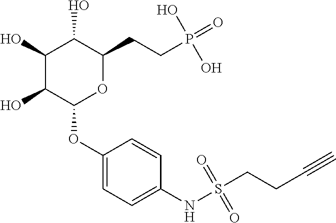 [SMILES: C#CCCS(=O)(=O)Nc1ccc(O[C@H]2O[C@H](CCP(=O)(O)O)[C@@H](O)[C@H](O)[C@@H]2O)cc1] |
| X37 | 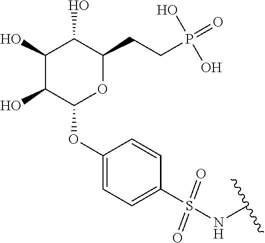 [SMILES: *NS(=O)(=O)c1ccc(O[C@H]2O[C@H](CCP(=O)(O)O)[C@@H](O)[C@H](O)[C@@H]2O)cc1] | 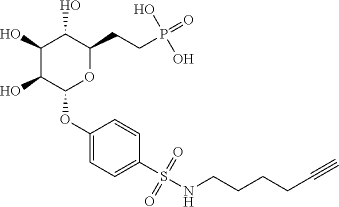 [SMILES: C#CCCCCNS(=O)(=O)c1ccc(O[C@H]2O[C@H](CCP(=O)(O)O)[C@@H](O)[C@H](O)[C@@H]2O)cc1] |
| X38 | 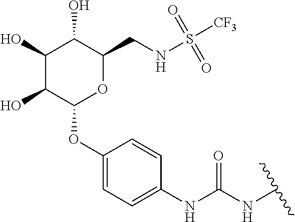 [SMILES: *NC(=O)Nc1ccc(O[C@H]2O[C@H](CNS(=O)(=O)C(F)(F)F)[C@@H](O)[C@H](O)[C@@H]2O)cc1] | 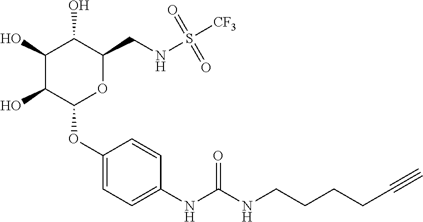 [SMILES: C#CCCCCNC(=O)Nc1ccc(O[C@H]2O[C@H](CNS(=O)(=O)C(F)(F)F)[C@@H](O)[C@H](O)[C@@H]2O)cc1] |
| X27* | 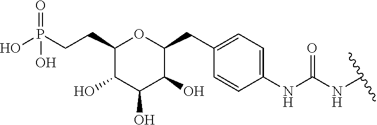 [SMILES: *NC(=O)Nc1ccc(C[C@@H]2O[C@H](CCP(=O)(O)O)[C@@H](O)[C@H](O)[C@@H]2O)cc1] | 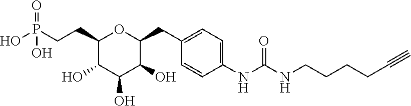 [SMILES: C#CCCCCNC(=O)Nc1ccc(C[C@@H]2O[C@H](CCP(=O)(O)O)[C@@H](O)[C@H](O)[C@@H]2O)cc1] |
| X39* | 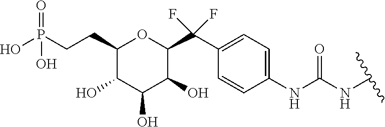 [SMILES: *NC(=O)Nc1ccc(C(F)(F)[C@@H]2O[C@H](CCP(=O)(O)O)[C@@H](O)[C@H](O)[C@@H]2O)cc1] | 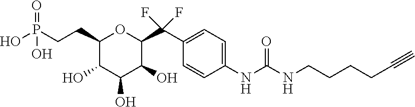 [SMILES: C#CCCCCNC(=O)Nc1ccc(C(F)(F)[C@@H]2O[C@H](CCP(=O)(O)O)[C@@H](O)[C@H](O)[C@@H]2O)cc1] |
| X39 | 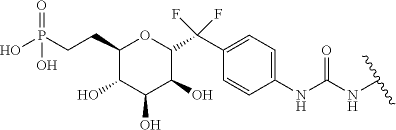 | 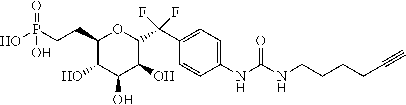 |
| X2* | 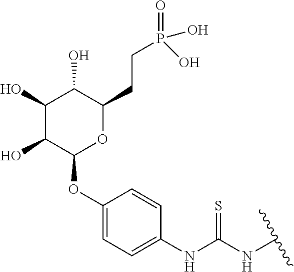 [SMILES: *NC(=S)Nc1ccc(O[C@@H]2O[C@H](CCP(=O)(O)O)[C@@H](O)[C@H](O)[C@@H]2O)cc1] | 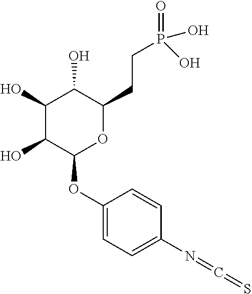 [SMILES: O=P(O)(O)CC[C@H]1O[C@@H](Oc2ccc(N=C=S)cc2)[C@@H](O)[C@@H](O)[C@@H]1O] |
| X3* | 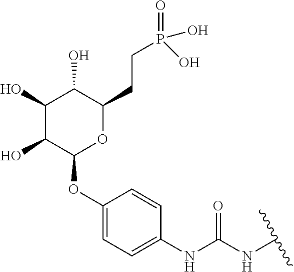 [SMILES: *NC(=O)Nc1ccc(O[C@@H]2O[C@H](CCP(=O)(O)O)[C@@H](O)[C@H](O)[C@@H]2O)cc1] | 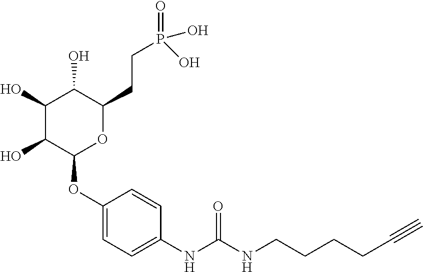 [SMILES: C#CCCCCNC(=O)Nc1ccc(O[C@@H]2O[C@H](CCP(=O)(O)O)[C@@H](O)[C@H](O)[C@@H]2O)cc1] |

Other M6PR binding moieties of interest and synthons or synthetic precursors thereof, are shown in Table 24. X101-X103 show compounds having a phosphate ester or thiophosphate ester head group. X109-X110 show exemplary compounds of formula (V). In some embodiments, such M6PR binding moieties are used in reference compounds for the assessment of compounds of formula (XII).

TABLE 24
Other Exemplary M6PR binding moieties and synthetic precursors

| Exemplary X for M6PR binding compounds | | Exemplary Synthetic precursors |
|---|---|---|
| # | Structure | Structure |
| X101 | 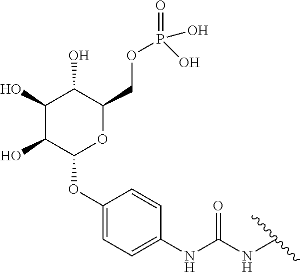 [SMILES: *NC(=O)Nc1ccc(O[C@H]2O[C@H](COP(=O)(O)O)[C@@H](O)[C@H](O)[C@@H]2O)cc1] | 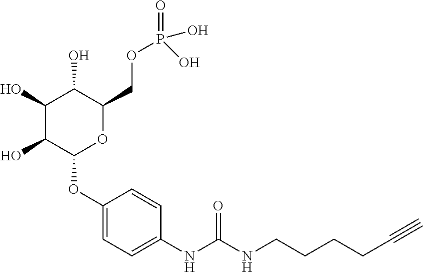 [SMILES: C#CCCCCNC(=O)Nc1ccc(O[C@H]2O[C@H](COP(=O)(O)O)[C@@H](O)[C@H](O)[C@@H]2O)cc1] |
| X102 | 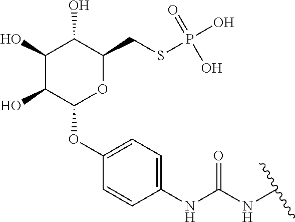 [SMILES: *NC(=O)Nc1ccc(O[C@H]2O[C@H](CSP(=O)(O)O)[C@@H](O)[C@H](O)[C@@H]2O)cc1] | 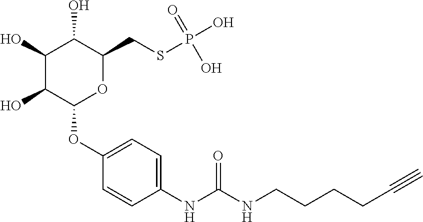 [SMILES: C#CCCCCNC(=O)Nc1ccc(O[C@H]2O[C@H](CSP(=O)(O)O)[C@@H](O)[C@H](O)[C@@H]2O)cc1] |
| X103 | 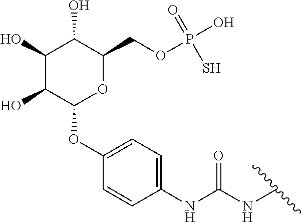 [SMILES: *NC(=O)Nc1ccc(O[C@H]2O[C@H](COP(=O)(O)S)[C@@H](O)[C@H](O)[C@@H]2O)cc1] | 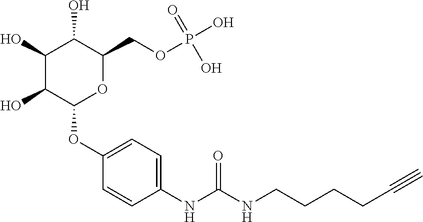 [SMILES: C#CCCCCNC(=O)Nc1ccc(O[C@H]2O[C@H](COP(=O)(O)O)[C@@H](O)[C@H](O)[C@@H]2O)cc1] |
| X104 | 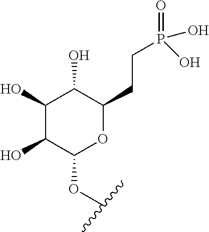 [SMILES: *O[C@H]1O[C@H](CCP(=O)(O)O)[C@@H](O)[C@H](O)[C@@H]1O] | 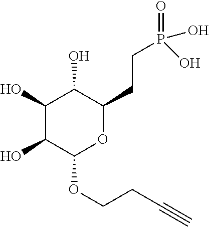 [SMILES: C#CCCO[C@H]1O[C@H](CCP(=O)(O)O)[C@@H](O)[C@H](O)[C@@H]1O] |
| X105 | 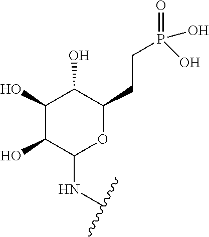 [SMILES: *NC1O[C@H](CCP(=O)(O)O)[C@@H](O)[C@H](O)[C@@H]1O] | 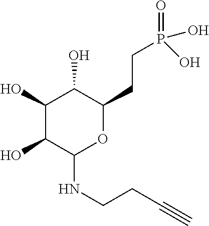 [SMILES: C#CCCNC1O[C@H](CCP(=O)(O)O)[C@@H](O)[C@H](O)[C@@H]1O] |
| X106 | 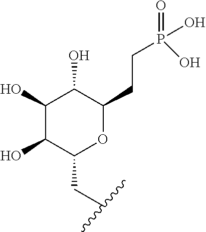 [SMILES: *C[C@H]1O[C@H](CCP(=O)(O)O)[C@@H](O)[C@H](O)[C@@H]1O] | 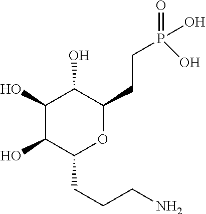 [SMILES: NCCC[C@H]1O[C@H](CCP(=O)(O)O)[C@@H](O)[C@H](O)[C@@H]1O] |
| X107 | 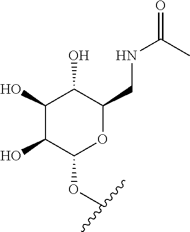 [SMILES: *O[C@H]1O[C@H](CNC(C)=O)[C@@H](O)[C@H](O)[C@@H]1O] | 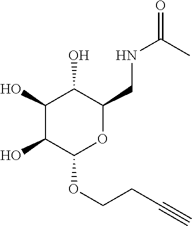 [SMILES: C#CCCO[C@H]1O[C@H](CNC(C)=O)[C@@H](O)[C@H](O)[C@@H]1O] |
| X108 | 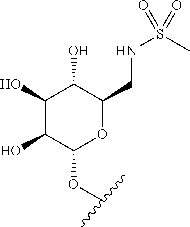 [SMILES: *O[C@H]1O[C@H](CNS(C)(=O)=O)[C@@H](O)[C@H](O)[C@@H]1O] | 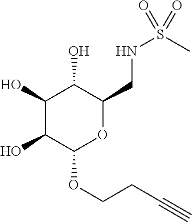 [SMILES: C#CCCO[C@H]1O[C@H](CNS(C)(=O)=O)[C@@H](O)[C@H](O)[C@@H]1O] |
| X109 | 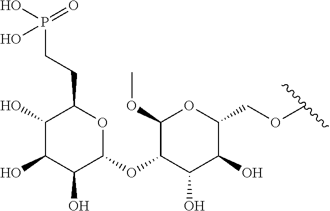 [SMILES: *OC[C@H]1O[C@H](OC)[C@@H](O[C@H]2O[C@H](CCP(=O)(O)O)[C@@H](O)[C@H](O)[C@@H]2O)[C@@H](O)[C@@H]1O] | 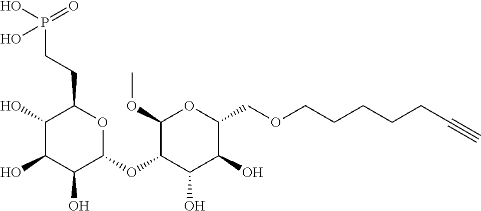 [SMILES: C#CCCCCCOC[C@H]1O[C@H](OC)[C@@H](O[C@H]2O[C@H](CCP(=O)(O)O)[C@@H](O)[C@H](O)[C@@H]2O)[C@@H](O)[C@@H]1O] |
| X110 | 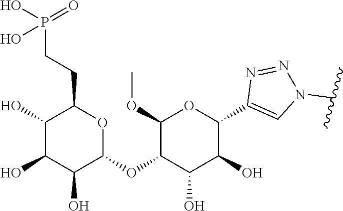 [SMILES: *n1cc([C@H]2O[C@H](OC)[C@@H](O[C@H]3O[C@H](CCP(=O)(O)O)[C@@H](O)[C@H](O)[C@@H]3O)[C@@H](O)[C@@H]2O)nn1] | 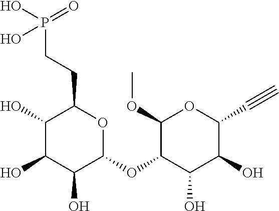 [SMILES: C#C[C@H]1O[C@H](OC)[C@@H](O[C@H]2O[C@H](CCP(=O)(O)O)[C@@H](O)[C@H](O)[C@@H]2O)[C@@H](O)[C@@H]1O] |

### Conjugates

The bifunctional molecules of this disclosure can be referred to as a conjugate, e.g., when the target binding moiety of interest is a polypeptide (e.g., in the form of B or Y—B) (e.g., as described herein, see Formula (Ia)). Such conjugates can be prepared by conjugation of a chemoselective ligation group of any one of the ligand moiety-linker compounds described herein with a compatible reactive group of a molecule B or Y—B. The compatible group of the molecule B or Y—B can be introduced by modification prior to conjugation, or can be a group present in the molecule. Alternatively, such conjugates can be prepared de novo, e.g., via modification of a B or Y—B molecule of interest starting material to introduce a linker, e.g., to which an ASGPR ligand moiety (X) can be attached. In some cases, the linking moiety between X-L and B or Y—B incorporates the residual group (e.g., Z of Formula (Ia)) that is the product of the chemoselective ligation chemistry.

As summarized above, in some embodiments of formula (I), the bifunctional molecule is a conjugate of formula (Ia):

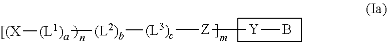

wherein:
Z is residual moiety resulting from the covalent linkage of a chemoselective ligation group of the linker to a compatible group of Y—B;
n is 1, 2, or 3; and
m is the average number of (XₙL) moieties conjugated to Y—B, wherein m is in the range from about 1 to about 80 (e.g., in is 1 to 20, 1 to 10, 1 to 8, 2 to 8, 3 to 6, or 4 to 5, or m is from 1 to 3, such as 1, 2 or 3).

In certain embodiments of formula (Ia), Z is a residual moiety resulting from the covalent linkage of a thiol-reactive chemoselective ligation group to one or more cysteine residue(s) of Y—B; or Z is a residual moiety resulting from the covalent linkage of an amine-reactive chemoselective ligation group to one or more lysine residue(s) of Y—B.

In certain embodiments of formula (Ia), Y—B is a chimeric fusion protein including a carrier polypeptide and a polypeptide that specifically binds the target autoantibody.

In certain embodiments of formula (I)-(Ia), Z is a residual moiety resulting from the covalent linkage of a chemoselective ligation moiety of Table 4.

In certain embodiments of the conjugates described herein, L is bonded through an amide bond (Z) to a lysine residue of the polypeptide. In certain embodiments of the conjugates described herein, L is bonded through a thioether bond (Z) to a cysteine residue of the polypeptide.

In certain embodiments, conjugation to the polypeptide may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide folding and assembly or alter polypeptide binding.

In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety. All such methodologies are contemplated for use in connection with making the conjugates described herein.

The loading of a ligand-linker moiety (e.g., Xn-L-) with respect to a polypeptide or protein construct (e.g., Y—B of a conjugate of formula (I)) refers to the number (a discrete or average loading) of ligand-linker moieties attached per molecule of polypeptide or protein construct. The loading is related to the concept of a drug to antibody ratio (“DAR”) in antibody drug conjugates. As used herein, the loading can be referred to as the linker to polypeptide ratio (“LPR”). The loading of the ligand-linker moiety (Xn-L-) with respect to a polypeptide bait-HSA protein construct (Y—B) of formula (I) described herein can be represented by the symbol “m”. The symbol “m” of formula (I) can refer to an average number of units of “Xn-L-” per polypeptide conjugate molecule, or alternatively, “m” can refer to a discrete number of units of “Xn-L-” per polypeptide conjugate molecule.

The number of “X” moieties per each unit of “Xn-L-” is represented by “n” in formula (I). As used herein, the terms “valency” and “multivalency” refer to a number of “X” moieties per unit (i.e., “n”). Thus, a bivalent ligand-linker moiety has n=2, and a trivalent ligand-linker moiety has n=3. It will be understood that the loading, or LPR, is not necessarily equivalent to the total number of “X” moieties per conjugate molecule, which is rather a combination of n and m. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and about four “Xn-L-” units per conjugate (m=4), there will be about 2×4=about 8 “X” moieties per conjugate. Accordingly, for the conjugates described herein, the total number of “X” moieties per conjugate molecule will be n×m.

LPR (loading) may range from 1 to 20 ligand-linker moieties per protein conjugate. The conjugates provided herein may include collections of polypeptides conjugated with a range of ligand-linker moieties, e.g., from 1 to 20. The number of ligand-linker moieties per polypeptide in preparations of the conjugate from conjugation reactions may be characterized by conventional means such as mass spectroscopy. It is understood that the definition of “m” as a discrete loading value, or as an average loading value has been determined in part by the nature of the chemoselective ligation chemistry and groups utilized in preparation of the conjugates. By way of example, stoichiometric thiol-maleimide conjugation chemistry can provide discrete conjugates via site specific conjugation to particular and specific target cysteine residues of interest. Alternatively, when an amine-specific conjugation chemistry is used, conjugation to one or up to several of the accessible lysine residues in the protein may occur, where the resulting conjugate preparation may contain several different species conjugates, and the loading can be represented by an average number. In some embodiments, the quantitative distribution of LPR (loading) in terms of “m” may also be determined. In some instances, separation, purification, and characterization of a homogeneous conjugate where “in” is a discrete value may be achieved by means such as electrophoresis.

In certain embodiments, the LPR for a conjugate provided herein ranges from 1 to 80. In certain embodiments, the LPR for a conjugate provided herein ranges from 1 to 20. In certain embodiments, the LPR for a conjugate provided herein ranges from 1 to 18. In certain embodiments, the LPR for a conjugate provided herein ranges from 1 to 15. In certain embodiments, the LPR for a conjugate provided herein ranges from 1 to 12. In certain embodiments, the LPR for a conjugate provided herein ranges from 1 to 10. In certain embodiments, the LPR for a conjugate provided herein ranges from 1 to 9. In certain embodiments, the LPR for a conjugate provided herein ranges from 1 to 8. In certain embodiments, the LPR for a conjugate provided herein ranges from 1 to 7. In certain embodiments, the LPR for a conjugate provided herein ranges from 1 to 6. In certain embodiments, the LPR for a conjugate provided herein ranges from 1 to 5. In certain embodiments, the LPR for a conjugate provided herein ranges from 1 to 4. In certain embodiments, the LPR for a conjugate provided herein ranges from 1 to 3. In certain embodiments, the LPR for a conjugate provided herein ranges from 2 to 12. In certain embodiments, the LPR for a conjugate provided herein ranges from 2 to 10. In certain embodiments, the LPR for a conjugate provided herein ranges from 2 to 9. In certain embodiments, the LPR for a conjugate provided herein ranges from 2 to 8. In certain embodiments, the LPR for a conjugate provided herein ranges from 2 to 7. In certain embodiments, the LPR for a conjugate provided herein ranges from 2 to 6. In certain embodiments, the LPR for a conjugate provided herein ranges from 2 to 5. In certain embodiments, the LPR for a conjugate provided herein ranges from 2 to 4. In certain embodiments, the LPR for a conjugate provided herein ranges from 3 to 12. In certain embodiments, the LPR for a conjugate provided herein ranges from 3 to 10. In certain embodiments, the LPR for a conjugate provided herein ranges from 3 to 9. In certain embodiments, the LPR for a conjugate provided herein ranges from 3 to 8. In certain embodiments, the LPR for a conjugate provided herein ranges from 3 to 7. In certain embodiments, the LPR for a conjugate provided herein ranges from 3 to 6. In certain embodiments, the LPR for a conjugate provided herein ranges from 3 to 5. In certain embodiments, the LPR for a conjugate provided herein ranges from 3 to 4.

In certain embodiments, the LPR for a conjugate provided herein ranges from 1 to about 8; from about 2 to about 6; from about 3 to about 5; from about 3 to about 4; or from about 4 to about 6. In certain embodiments, the LPR for a conjugate ranges from about 3.1 to about 3.9; from about 3.2 to about 3.8; from about 3.2 to about 3.7; from about 3.2 to about 3.6; from about 3.3 to about 3.8; or from about 3.3 to about 3.7. In certain embodiments, the LPR for a conjugate ranges from about 4.1 to about 4.9; from about 4.2 to about 4.8; from about 4.2 to about 4.7; from about 4.2 to about 4.6; from about 4.3 to about 4.8; or from about 4.3 to about 4.7.

In certain embodiments, the LPR for a conjugate provided herein is about 1, about 2, about 3, about 4, about 5, about 6, about 7, about 8, about 9, about 10, about 11, about 12, or more. In some embodiments, the LPR for a conjugate provided herein is about 3.1, about 3.2, about 3.3, about 3.4, about 3.5, about 3.6, about 3.7, about 3.8, or about 3.9. In some embodiments, the LPR for a conjugate provided herein is about 4.1, about 4.2, about 4.3, about 4.4, about 4.5, about 4.6, about 4.7, about 4.8, or about 4.9.

In some embodiments, the LPR for a conjugate provided herein ranges from 2 to 20, 2 to 19, 2 to 18, 2 to 17, 2 to 16, 2 to 15, 2 to 14, or 2 to 13. In some embodiments, the LPR for a conjugate provided herein ranges from 3 to 20, 3 to 19, 3 to 18, 3 to 17, 3 to 16, 3 to 15, 3 to 14, or 3 to 13. In some embodiments, the LPR for a conjugate provided herein is about 1. In some embodiments, the LPR for a conjugate provided herein is about 2. In some embodiments, the LPR for a conjugate provided herein is about 3. In some embodiments, the LPR for a conjugate provided herein is about 4. In some embodiments, the LPR for a conjugate provided herein is about 5. In some embodiments, the LPR for a conjugate provided herein is about 6. In some embodiments, the LPR for a conjugate provided herein is about 7. In some embodiments, the LPR for a conjugate provided herein is about 8. In some embodiments, the LPR for a conjugate provided herein is about 9. In some embodiments, the LPR for a conjugate provided herein is about 10.

In certain embodiments, fewer than the theoretical maximum of units are conjugated to the polypeptide during a conjugation reaction. A polypeptide may contain, for example, lysine residues that do not react with the compound or linker reagent. Generally, for example, polypeptides do not contain many free and reactive cysteine thiol groups which may be linked to a ligand-linker moiety; indeed many cysteine thiol residues in polypeptides or proteins exist as disulfide bridges. In certain embodiments, a polypeptide may be reduced with a reducing agent such as dithiothreitol (DTT) or tricarbonylethylphosphine (TCEP), under partial or total reducing conditions, to generate reactive cysteine thiol groups. In certain embodiments, a polypeptide is subjected to denaturing conditions to reveal reactive nucleophilic groups such as lysine or cysteine. In some embodiments, the ligand-linker moiety is conjugated via a lysine residue on the polypeptide. In some embodiments, the ligand-linker moiety is conjugated via a cysteine residue on the polypeptide.

The loading (LPR) of a conjugate may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the polypeptide, such that the number and position of cysteine residues is modified for control of the number and/or position of linker-drug attachments (such as for thiomabs prepared as disclosed in WO2006/034488.

It is to be understood that the preparation of the conjugates described herein may result in a mixture of conjugates with a distribution of one or more ligand-linker moieties attached to a polypeptide. Individual conjugate molecules may be identified in the mixture by mass spectroscopy and separated by HPLC, e.g., hydrophobic interaction chromatography, including such methods known in the art. In certain embodiments, a homogeneous conjugate with a single LPR (loading) value may be isolated from the conjugation mixture by electrophoresis or chromatography.

In certain embodiments of the conjugate of formula (I) m is 1 to 20, such as 2 to 10, 2 to 8, or 2 to 6. In certain cases, m is 10 or less. In certain cases, m is 2 to 8. In certain cases, m is 2 to 6. In certain cases, m is an average loading of about 4.

Polypeptides that Bind Extracellular Target Molecules

As summarized above, the bifunctional molecules of this disclosure can include a extracellular target molecule-binding moiety that specifically binds the target molecule. In some embodiments, the extracellular target molecule-binding moiety B is conjugated or linked connected to a carrier polypeptide Y.

By “extracellular target molecule” is meant a soluble molecule external to the cell membranes of any cells in the vicinity of the soluble molecule. The extracellular target molecule may be any extracellular target molecule which is desired for targeted degradation via the endosomal/lysosomal pathway. In certain embodiments, the extracellular target molecule binding moiety (B) specifically binds a target autoantibody.

In some embodiments, the target binding polypeptide is a protein antigen that contains the sites of binding (e.g., epitopes) of the disease-causing autoantibodies. In some embodiments, the target binding polypeptide is the primary immunogenic or epitope-containing region of the protein to which the autoantibodies predominantly bind in vivo.

### Antibody

In some embodiments, the extracellular target molecule is an antibody, e.g., an antibody (Ab) that specifically binds a cell surface molecule or different extracellular molecule. The target antibody can be an extracellular autoantibody associated with a disease or condition of interest.

In some embodiments, the extracellular target molecule is a human immunoglobulin A (IgA). In some embodiments, the IgA is a particular antibody that plays a crucial role in the immune function of mucous membranes.

In the blood, IgA interacts with an Fc receptor called CD89 expressed on immune effector cells, to initiate inflammatory reactions. Aberrant IgA expression has been implicated in a number of autoimmune and immune-mediated disorders. In some embodiments, the target is a human immunoglobulin G (IgG). The Fc regions of IgGs include a conserved N-glycosylation site at asparagine 297 in the constant region of the heavy chain. Various N-glycans can be attached to this site. The N-glycan IgG composition has been linked to several autoimmune, infectious and metabolic diseases. In addition, overexpression of IgG4 has been associated with IG4-related diseases.

In some embodiments, the target is human immunoglobulin E (IgE). IgE is a type of immunoglobulin that plays an essential role in type I hypersensitivity, which can manifest into various allergic diseases and conditions.

In some embodiments, the extracellular target molecule is an autoantibody. Autoantibodies are generally associated with autoimmune diseases. Non-limiting examples of the extracellular target molecule include rheumatoid factor (RF), antinuclear antibody (ANA), antineutrophil cytoplasmic antibodies (ANCA, aPR3, MPO), anti-double stranded DNA (dsDNA), anticentromere antibodies (ACA), antihistone antibodies, cyclic citrullinated peptide antibodies (CCP), extractable nuclear antigen antibodies (e.g., anti-SS-A (Ro) and anti-SS—B (La), anti-RNP, anti-Jo-1, anti-Sm, Scl-70), cardiolipin antibodies, beta-2 glycoprotein I antibodies, antiphospholipid antibodies (APA), lupus anticoagulants (LA), diabetes-related autoantibodies, anti-tissue transglutaminase (anti-tTC), anti-gliadin antibodies (AGA), intrinsic factor antibodies, parietal cell antibodies, thyroid autoantibodies (e.g., TPO antibodies, TSH receptor antibodies), smooth muscle antibodies (SMA), antimitochondrial antibodies (AMA), liver kidney microsome type 1 antibodies (LKM-1), anti-glomerular basement membrane (GBM), acetylcholine receptor (aChR) antibodies, N-methyl-D-aspartate (NMDAR) antibodies, myasthenia gravis antibodies (anti-aChR, anti-MuSK), antibodies related to autoimmune nephropathies (e.g., anti-IgA and anti-PLA2R), antibodies related to pregnancy induced precclampsia (anti-AT1R), encephalitis antibodies (anti-NMDAR), and pemphigus antibodies (e.g., anti-BP180 and anti-BP230). In some embodiments, the target molecule is a TSH receptor autoantibody. In some embodiments, the target molecule is a MuSK antibody. In some embodiments, the target molecule is an aChR antibody. In some embodiments, the target molecule is a BP180 antibody. In some embodiments, the target molecule is a PR3 antibody.

In some embodiments, the extracellular target molecule is a neutralizing antibody or an anti-drug antibody. A “neutralizing antibody” refers to an antibody that defends a cell from pathogen (e.g., viral antigen or protein) or infectious particles. Non-limiting examples of pathogen or infectious particles are corona virus, SARS-CoV-1, SARS-CoV-2, cytomegalovirus (CMV), hepatitis b virus (HBV), rabies virus, measles virus, respiratory syncytial vircytous (RSV), and Diphtheria antitoxin. As an illustration, neutralizing antibodies exert their effects by binding directly to virus particles and blocking subsequent interactions with receptors or by inhibiting post entry events such as viral uncoating and replication. In another example, neutralizing antibodies can neutralize the toxic effects of bacterial toxins (Diphtheria antitoxin).

In some embodiments, the extracellular target molecule is an anti-drug antibody. Anti-drug antibodies may bind to a drug or therapeutic protein product. In some embodiments, an anti-drug antibody is an antibody that specifically binds to the idiotope of another antibody, generally an antibody drug. An idiotope corresponds to a region within the Fv region binding to the paratope of a different antibody. Anti-drug antibodies may be used for cancer treatment or management.

### Extracellular Target Molecule Binding Moiety (B or Y—B)

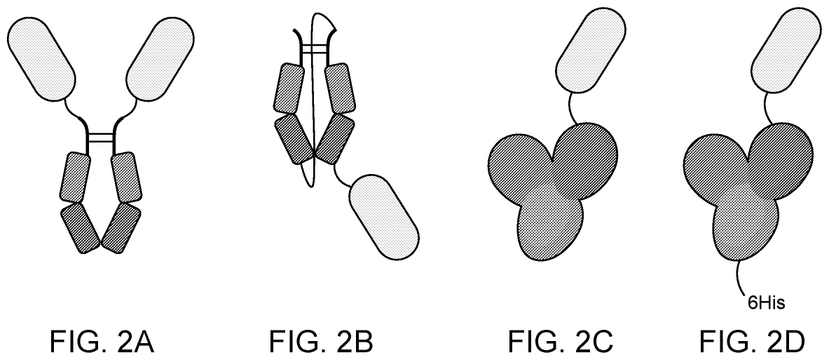

In some embodiments, the extracellular target molecule binding moiety includes a polypeptide B that specifically binds an extracellular target molecule (e.g., autoAb-A, autoAb-B. or autoAb-C) and optionally a carrier polypeptide (Y). Shown is an illustration of an extracellular target molecule binding moiety Y—B. FIG. 2 illustrates exemplary extracellular target molecule binding moieties described herein. In some embodiments, the carrier polypeptide Y comprises a protein domain. In some embodiments, the polypeptide B and the carrier polypeptide Y are fused directly. In some embodiments, the polypeptide B and the carrier polypeptide Y are fused indirectly via a spacer domain. In some embodiments, the extracellular target molecule binding moiety (Y—B) is a chimeric protein having a polypeptide encoded by a nucleic acid molecule of the polypeptide B and the carrier polypeptide Y. In some embodiments, the carrier polypeptide Y is a human serum albumin (HSA) conjugated to the polypeptide B at a cysteine residue on HSA to form an extracellular target molecule polypeptide binding moiety.

In some embodiments, the carrier polypeptide (Y) is a Fe fragment generated from the heavy chain constant region of an immunoglobulin, a Fc monomer, a Fc dimer, or fragments thereof (e.g., synthetic peptides). Shown is an exemplary FEc mono carrier polypeptide Y. In some embodiments, the carrier polypeptide (Y) Fc is conjugated to a single variable domain on a heavy chain (VHH) antibody, a nanobody, a domain, or a fragment thereof, to form an VHH antigen fusion. In some embodiments, the carrier polypeptide (Y) Fc is conjugated to an antigen to form an Fc-antigen fusion. In various embodiments, the Fc fragment is conjugated to polypeptide B at a cysteine residue on the Fc fragment.

### Carrier Polypeptide (Y)

A number of strategies, such as attachment of a carrier, for imparting desirable pharmacokinetic properties to a peptide or protein (e.g., avoidance of rapid renal clearance, and/or optimizing Fe receptor mediated recycling) can be adapted for use in the bifunctional molecules of this disclosure that contain a target binding polypeptide.

The target binding polypeptide can be connected to a carrier polypeptide Y that imparts one or more desirable properties onto the resulting bifunctional molecule (e.g., increase in vivo stability and/or half-life), and/or provides for conjugation sites to a ligand-linker moiety, e.g., without disrupting the autoantibody binding properties of the target binding polypeptide.

In some embodiments, the target binding polypeptide is synthetically linked to a carrier polypeptide Y. In some embodiments of formula (I), the carrier polypeptide Y is conjugated to a polypeptide (B) via a bifunctional linker.

In some embodiments, the target binding polypeptide is linked to a carrier polypeptide Y as part of a protein construct, such as a chimeric fusion protein. In some embodiments, the polypeptide B and the carrier polypeptide Y are fused directly to each other, e.g., via N-terminal to C-terminal fusion, or C-terminal to N-terminal fusion. In some embodiments, the polypeptide B and the carrier polypeptide Y are fused indirectly via a spacer domain. In some embodiments, the Y—B of formula (I) is a chimeric protein having a polypeptide encoded by a nucleic acid molecule that encodes for both a polypeptide B and the carrier polypeptide Y.

The carrier polypeptide (Y) can be a serum protein or domain of a serum protein. In some embodiments, the serum protein is an albumin. In some embodiments, the serum protein is an immunoglobulin. In some embodiments, the carrier polypeptide (Y) is an engineered serum protein or domain thereof. The carrier polypeptide (Y) can be prepared synthetically, or recombinantly.

FIG. 2 illustrates an exemplary protein (Y—B) that includes a target binding polypeptide (e.g., autoantibody “bait”) linked to carrier polypeptide (e.g., HSA carrier protein). In some embodiments, the Y—B is referred to as a protein construct, where the engineered chimeric protein is amenable to preparation using recombinant protein technology.

In some embodiments, the carrier polypeptide (Y) is a human serum albumin (HSA) or fragments thereof (e.g., synthetic peptides).

In some embodiments, the carrier polypeptide (Y) is HSA domain or fragments thereof (e.g., synthetic peptides).

In some embodiments, the carrier polypeptide (Y) is albumin binding domain or fragments thereof (e.g., synthetic peptides).

In some embodiments, the carrier polypeptide (Y) is Fc (monomer) or fragments thereof (e.g., synthetic peptides).

In some embodiments, the carrier polypeptide (Y) is Fc (dimer) or fragments thereof (e.g., synthetic peptides).

In some embodiments, the carrier polypeptide (Y) is linked to the extracellular target molecule (B) via a linker. The linker may comprise a non-peptide linking moiety.

### Albumin

The carrier polypeptide can be a serum albumin protein or domain or subdomain thereof, or fragment thereof. In some embodiments, the carrier polypeptide is human serum albumin (HSA).

Albumins generally have a long plasma half-life (e.g., about 3 weeks) and can provide a scaffold to which bioactive molecules can be attached or fused. HSA has a long serum half-life in humans that is attributed in part to its interaction with neonatal Fc receptor (FcRn). HSA (molecular mass 66.5 kDa) includes three structurally similar and flexible domains: I (residues 1-195), II (196-383) and III (384-585). Each domain (D) is composed of two subdomains A and B (e.g., DIA [5-105], DIB [119-195], DIIA [196-292], DIIB [314-383], DIIIA [384-491], DIIIB [510-582], e.g., as referenced to SEQ ID NO: 2 of WO2017/029407) with common structural motifs.

In some embodiments, the carrier protein is an HSA variant having enhanced FcRn binding. FcRn binds to the C-terminal end of DIII of HSA and protects albumin from intracellular degradation. For instance, a single amino acid substitution within HSA DIII (K537P) shows 12-fold improved binding affinity to FcRn, which translates into longer half-life. Various amino acid residues of albumin located in Domain I or Domain II also affect HSA interaction with FcRn (e.g., WO 2013/135896 describes albumin variants having one or more alterations in Domain I and one or more alterations in Domain III; WO 2015/036579 describes albumin variants having one or more alterations in Domain II).

Any convenient albumin protein can be a parent for an albumin variant that can be utilized as a carrier polypeptide. As an example, human serum albumin (HSA) such as AAA98797, P02768-1, SEQ ID NO. 25 (mature HSA), or SEQ ID NO. 24 (immature HSA).

In some embodiments, other albumins are utilized as a carrier polypeptide. Such other albumins include, but are not limited to, primate serum albumin (e.g., chimpanzee serum albumin. XP_517233.2), gorilla serum albumin or macaque serum albumin (e.g., NP_001 182578), rodent serum albumin (e.g., hamster serum albumin. A6YF56), guinea pig serum albumin (e.g., Q6WDN9-1), mouse serum albumin (e.g., AAH49971, P07724-1 Version 3) and rat serum albumin (e.g., AAH85359, P02770-1 Version 2), bovine serum albumin (e.g., cow serum albumin P02769-1), equine serum albumin such as horse serum albumin (e.g., P35747-1), or donkey serum albumin (e.g., Q5XLE4-1), rabbit serum albumin (e.g., P49065-1 Version 2), goat serum albumin (e.g., ACF10391), sheep serum albumin (e.g., P14639-1), dog serum albumin (e.g., P49822-1), chicken serum albumin (e.g., P19121-1 Version 2) and pig serum albumin (e.g., P08835-1 Version 2) or a polypeptide having at least 70%, 75%, 80%, 85%, 90%, 95%, 96%, 97%, 98%, 99%, 99.2%, 99.4%, 99.6%, or at least 99.8% amino acid sequence identity to such an albumin. Non-mammalian albumins of interest include ovalbumin (e.g., P01012.pro: chicken ovalbumin; 073860. Pro: turkey ovalbumin).

In some embodiments, albumin, a fragment thereof, or conjugation-competent albumin variant, or albumin part of a fusion polypeptide or conjugate comprising albumin or a fragment thereof has a polypeptide sequence having at least 80%, 85%, 90%, 91%, 92%, 93%, 94%, 95%, 96%, 97%, 98%, 99%, or 100% sequence identity to SEQ ID NO: 24 (e.g., P02768-1) below, where Domain I is in bold, Domain II is underlined, and Domain III is in italic:

(SEQ ID NO: 24)
MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLV AASQAALGL.

In some embodiments, albumin, a fragment thereof, or conjugation-competent albumin variant, or albumin part of a fusion polypeptide or conjugate comprising albumin or a fragment thereof has a polypeptide sequence having at least 80%, 85%, 90%, 91%, 92%, 93%, 94%, 95%, 96%, 97%, 98%, 99%, or 100% sequence identity to SEQ ID NO: 25 below:

(SEQ ID NO: 25)
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL.

In some embodiment, the albumin, a fragment thereof, or conjugation-competent albumin variant, or variant thereof of the bifunctional molecule has a sequence identity to the sequence of HSA shown in SEQ ID NO. 7 of at least 80%, 85%, 90%, 91% 92%, 93%, 94%, 95%, 96%, 97%, 98%, 99%, or 100%. It is preferred that the albumin maintains at least one of the major properties of albumin or a similar tertiary structure as an albumin, such as HSA. A functional fragment of albumin may have a sequence identity of at least 60%, 70%, 80%, 85%, 90%, 91%, 92%, 93%, 94%, 95%, 96%, 97%, 98%, 99%, 99.2%, 99.4%, 99.6%, 99.8%, or 100% to the sequence of HSA Domain III as shown in SEQ ID NO.26, or to the sequence of HSA Domain II and Domain III as shown in SEQ ID NO. 27, or to a molecule consisting of or comprising two copies of Domain III (e.g., SEQ ID NO. 28), or to a molecule consisting of or comprising three copies of Domain III (e.g., SEQ ID NO. 29), or to a molecule consisting of or comprising Domain I and two copies of Domain III (e.g., SEQ ID NO. 30).

TABLE 25

| Name | Sequence | SEQ ID NO. |
|---|---|---|
| HSA | VEEPQNLIKQNCELFEQLGEYKFQNALLVRYTKKVPQVSTPTL | 26 |
| Domain | VEVSRNLGKVGSKCCKHPEAKRMPCAEDYLSVVLNQLCVLH | (19 of |
| III | EKTPVSDRVTKCCTESLVNRRPCFSALEVDETYVPKEFNAETF | WO2017/02940) |
| | TFHADICTLSEKERQIKKQTALVELVKHKPKATKEQLKAVMD | |
| | DFAAFVEKCCKADDKETCFAEEGKKLVAASQAALGL | |
| HSA | DAHKSEVAHRFKDLGEENFKALVLIAFAQYLQQCPFEDHVKL | 27 |
| Domain | VNEVTEFAKTCVADESAENCDKSLHTLFGDKLCTVATLRETY | (21 of |
| II and | GEMADCCAKQEPERNECFLQHKDDNPNLPRLVRPEVDVMCT | WO2017/02940) |
| Domain | AFHDNEETFLKKYLYEIARRHPYFYAPELLFFAKRYKAAFTEC | |
| III | CQAADKAACLLPKLDELRDEGKASSAVEEPQNLIKQNCELFE | |
| | QLGEYKFQNALLVRYTKKVPQVSTPTLVEVSRNLGKVGSKCC | |
| | KHPEAKRMPCAEDYLSVVLNQLCVLHEKTPVSDRVTKCCTES | |
| | LVNRRPCFSALEVDETYVPKEFNAETFTFHADICTLSEKERQIK | |
| | KQTALVELVKHKPKATKEQLKAVMDDFAAFVEKCCKADDK | |
| | ETCFAEEGKKLVAASQAALGL | |
| HSA 2 | VEEPQNLIKQNCELFEQLGEYKFQNALLVRYTKKVPQVSTPTL | 28 |
| copies | VEVSRNLGKVGSKCCKHPEAKRMPCAEDYLSVVLNQLCVLH | (22 of |
| Domain | EKTPVSDRVTKCCTESLVNRRPCFSALEVDETYVPKEFNAETF | WO2017/02940) |
| III | TFHADICTLSEKERQIKKQTALVELVKHKPKATKEQLKAVMD | |
| | DFAAFVEKCCKADDKETCFAEEGKKLVAASQAALGLVEEPQ | |
| | NLIKQNCELFEQLGEYKFQNALLVRYTKKVPQVSTPTLVEVSR | |
| | NLGKVGSKCCKHPEAKRMPCAEDYLSVVLNQLCVLHEKTPV | |
| | SDRVTKCCTESLVNRRPCFSALEVDETYVPKEFNAETFTFHAD | |
| | ICTLSEKERQIKKQTALVELVKHKPKATKEQLKAVMDDFAAF | |
| | VEKCCKADDKETCFAEEGKKLVAASQAALGL | |
| HSA 3 | VEEPQNLIKQNCELFEQLGEYKFQNALLVRYTKKVPQVSTPTL | 29 |
| copies | VEVSRNLGKVGSKCCKHPEAKRMPCAEDYLSVVLNQLCVLH | (23 of |
| Domain | EKTPVSDRVTKCCTESLVNRRPCFSALEVDETYVPKEFNAETF | WO2017/02940) |
| III | TFHADICTLSEKERQIKKQTALVELVKHKPKATKEQLKAVMD | |
| | DFAAFVEKCCKADDKETCFAEEGKKLVAASQAALGLVEEPQ | |
| | NLIKQNCELFEQLGEYKFQNALLVRYTKKVPQVSTPTLVEVSR | |
| | NLGKVGSKCCKHPEAKRMPCAEDYLSVVLNQLCVLHEKTPV | |
| | SDRVTKCCTESLVNRRPCFSALEVDETYVPKEFNAETFTFHAD | |
| | ICTLSEKERQIKKQTALVELVKHKPKATKEQLKAVMDDFAAF | |
| | VEKCCKADDKETCFAEEGKKLVAASQAALGLVEEPQNLIKQN | |
| | CELFEQLGEYKFQNALLVRYTKKVPQVSTPTLVEVSRNLGKV | |
| | GSKCCKHPEAKRMPCAEDYLSVVLNQLCVLHEKTPVSDRVT | |
| | KCCTESLVNRRPCFSALEVDETYVPKEFNAETFTFHADICTLSE | |
| | KERQIKKQTALVELVKHKPKATKEQLKAVMDDFAAFVEKCC | |
| | KADDKETCFAEEGKKLVAASQAALGL | |
| HSA | DAHKSEVAHRFKDLGEENFKALVLIAFAQYLQQCPFEDHVKL | 30 |
| Domain I | VNEVTEFAKTCVADESAENCDKSLHTLFGDKLCTVATLRETY | (24 of |
| and 2 | GEMADCCAKQEPERNECFLQHKDDNPNLPRLVRPEVDVMCT | WO2017/02940) |
| copies | AFHDNEETFLKKYLYEIARRHPYFYAPELLFFAKRYKAAFTEC | |
| Domain | CQAADKAACLLPKLDELRDEGKASSAVEEPQNLIKQNCELFE | |
| III | QLGEYKFQNALLVRYTKKVPQVSTPTLVEVSRNLGKVGSKCC | |
| | KHPEAKRMPCAEDYLSVVLNQLCVLHEKTPVSDRVTKCCTES | |
| | LVNRRPCFSALEVDETYVPKEFNAETFTFHADICTLSEKERQIK | |
| | KQTALVELVKHKPKATKEQLKAVMDDFAAFVEKCCKADDK | |
| | ETCFAEEGKKLVAASQAALGLVEEPQNLIKQNCELFEQLGEY | |
| | KFQNALLVRYTKKVPQVSTPTLVEVSRNLGKVGSKCCKHPEA | |
| | KRMPCAEDYLSVVLNQLCVLHEKTPVSDRVTKCCTESLVNRR | |
| | PCFSALEVDETYVPKEFNAETFTFHADICTLSEKERQIKKQTAL | |
| | VELVKHKPKATKEQLKAVMDDFAAFVEKCCKADDKETCFAE | |
| | EGKKLVAASQAALGL | |

The human serum albumin (HSA) polypeptide chain has 35 cysteine residues, which form 17 disulphide bonds and one unpaired (free) cysteine at position 34 located in DI of the mature protein. The albumin, a fragment thereof, or conjugation-competent albumin variant, or albumin part of a fusion polypeptide or conjugate comprising albumin or a fragment thereof according to the invention, when folded, may have several, for example at least 1, 2, 3, 4, 5, 6, 7, 8, 9, 10, 11, 12, 13, 14, 15, 16 and suitably all 17, of the native disulphide bonds of the polypeptide of SEQ ID NO. 25.

Albumin carrier polypeptides which can be adapted for use in the bifunctional molecules of this disclosure are described in WO2017/029407, WO 2013/135896, WO 2015/036579, the disclosures of which are herein incorporated by reference in their entireties.

Conjugation of molecules to the free thiol on cysteine-34 of HSA can provide site-selective conjugation. To increase loading potential, recombinant HSA (rHSA) variants can be engineered to contain additional free, conjugation-competent cysteines.

The term “thio-albumin” is used to describe an albumin variant which comprises one or more (e.g. several) unpaired cysteine residues, particularly an albumin variant in which one or more (e.g. several) of the unpaired cysteine residues does not occur in a naturally occurring variant of an albumin. A thio-albumin refers to a conjugation-competent albumin. The rHSA variant can be a thio-albumin or a conjugation-competent albumin. The rHSA may have one or more free, conjugation-competent cysteines introduced in DI, DII and/or DIII. In some embodiments, the rHSA variant has at least one free, conjugation-competent cysteines introduced in DI or DII. In some embodiments, the rHSA variant has at least one free, conjugation-competent cysteine introduced in DI and DIII. In some embodiments, the rHSA variant has a conjugation site at Cys34 and at least one or more free, conjugation-competent cysteines introduced in DI, DII, and/or DIII.

In some embodiments, the carrier polypeptide is a rHSA variant having a conjugation position at Cys34 and one or more conjugation-competent cysteines introduced at positions selected from K93, A226, E230, I271, E294, E358, L24, F49, V54, D56, A92, Q94, E97, H128, F156, E227, D237, K240, D259, K262, N267, Q268, L275, L284, K317, A322, E333, D340, E354, K359, A362, E382, and L398 of SEQ ID NO. 25. The free, conjugation-competent cysteines may be introduced by substitution of an amino acid other than cysteine at any one of positions corresponding to or equivalent to any of residues selected from K93, A226, E230, I271, E294, E358, L24, F49, V54, D56, A92, Q94, E97, H128. F156. E227, D237, K240, D259, K262, N267, Q268, L275, L284, K317, A322, E333, D340, E354, K359, A362. E382, and L398 of SEQ ID NO. 25, or by insertion of a cysteine at a position adjacent to N- or C-side of an amino acid corresponding to a position equivalent to any of residues selected from K93, A226, E230, I271, E294, E358, L24. F49, V54. D56, A92, Q94, E97, H128, F156, E227, D237, K240, D259, K262, N267, Q268, L275, L284, K317, A322. E333, D340, E354, K359, A362, E382, and L398 of SEQ ID NO. 25. In some embodiments, at least one or more free, conjugation-competent cysteines is introduced at one or more positions corresponding to or equivalent to any of residues selected from C34, V54, H128, K240, and K262.

In some embodiments, the carrier polypeptide is a rHSA variant that exhibits up to 95% monomeric stability and retains hFcRn engagement compared with a wildtype unconjugated control (e.g., as demonstrated by Biolayer Interferometry). In some embodiments, introduction of the free, conjugation-competent cysteines into the rHSA variant does not interfere with FcRN binding. In some embodiments, introduction of the free, conjugation-competent cysteines maintains the albumin half-life in circulating blood.

### Immunoglobulin

In some embodiments, the carrier polypeptide is an Fe fragment, a monomer, a dimer, a domain, or fragments thereof. Fc fragments contain the CH2 and CH3 domains and part of the hinge region held together by one or more disulfides and noncovalent interactions. Fc and Fc5μ fragments are produced from fragmentation of IgG and IgM, respectively. Fc fragments are derived from the heavy chain constant region of an immunoglobulin. In some embodiments, Fc fragments are derived from IgG immunoglobulins of any subclass (e.g., IgG1, IgG2, IgG3, IgG4). In preferred embodiments, Fc fragments are derived from human IgG1.

The term “Fe dimer” refers to an Fe fragment containing two CH2-CH3 chains. The dimer is typically about 54k Da. An “Fe monomer” is generally half the size of the dimer (e.g., about 27k Da). Methods for generating Fe dimers and monomers are described in Wang et al. Engineering soluble monomeric IgG1 Fc with significantly decreased non-specific binding. Front. Immunol. (2017) 8:1545; Ying et al. Soluble monomeric IgG1 Fc. J. Biol Chem (2012) 287(23): 19399-408, each of which is incorporated herein by reference in its entirety.

In some embodiments, the Fc fragment has a polypeptide sequence having at least 80%, 85%, 90%, 91%, 92%, 93%, 94%, 95%, 96%, 97%, 98%, 99%, or 100% sequence identity to SEQ ID NO. 31 (human IgG1 hinge-Fc) below:

(SEQ ID NO: 31)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the Fc fragment is a variant of a human IgG Fc region. In some embodiments, the Fc fragment is a variant of human IgG Fc comprising one or more amino acid substitutions in the CH2 and/or CH3 domains.

In some embodiments, the carrier polypeptide (Y) is an Fc fragment generated from the heavy chain constant region of an immunoglobulin, an Fc monomer, an Fc dimer, or fragments thereof. In some embodiments, the carrier polypeptide (Y) Fc is conjugated to an antibody fragment comprising an antigen-binding region (e.g., nanobody, scFv, VHH) to form an engineered antibody. In some embodiments, the carrier polypeptide (Y) Fc is conjugated to an antigen to form an Fc-antigen fusion. In various embodiments, the Fc fragment is conjugated to polypeptide B at a cysteine residue on the Fc fragment.

In specific embodiments, the carrier polypeptide is a Fc fragment, a monomer, a dimer, a domain, or fragments thereof.

Fe fragments contain the CH2 and CH3 region and part of the hinge region held together by one or more disulfides and noncovalent interactions. Fe and Fc5p fragments are produced from fragmentation of IgG and IgM, respectively. The term Fe is derived from the ability of these antibody fragments to crystallize. Fc fragments are usually generated from the heavy chain constant region of an immunoglobulin.

### Polypeptide (B)

In some embodiments, the polypeptide (B) specifically binds a target antibody (e.g., an extracellular antibody associated with a disease or condition of interest). The target antibody can be an autoantibody. The target antibody can be a neutralizing antibody or an anti-drug antibody. In some embodiments, the polypeptide (B) comprises an antigen of the target antibody, or fragment thereof (e.g., synthetic peptide). In such example, the antigen can be an autoantigen. In accordance to various embodiments, the polypeptide (B) comprises a protein domain, such as an antibody, an antigen, or fragments thereof.

### Autoantibody

In specific embodiments, the polypeptide B specifically binds an extracellular target molecule that is associated with one or more autoimmune disorders or diseases. In some embodiments, the polypeptide B is an autoantibody described herein. Non-limiting examples of autoantibodies include rheumatoid factor (RF), antinuclear antibody (ANA), Antineutrophil Cytoplasmic Antibodies (ANCA), Anti-Double Stranded DNA (anti-dsDNA), Anticentromere Antibodies (ACA), Antihistone Antibodies, Cyclic Citrullinated Peptide Antibodies (CCP), Extractable Nuclear Antigen Antibodies (e.g., anti-SS-A (Ro) and anti-SS—B (La), anti-RNP, anti-Jo-1, anti-Sm, Scl-70), Cardiolipin Antibodies, Beta-2 Glycoprotein 1 Antibodies, Antiphospholipid Antibodies (APA). Lupus anticoagulants (LA), Diabetes-related Autoantibodies, Anti-Tissue Transglutaminase (anti-tTC), Anti-Gliadin Antibodies (AGA), Intrinsic Factor Antibodies, Parietal Cell Antibodies, Thyroid Autoantibodies (e.g., anti-TPO, TSH receptor antibodies), Smooth Muscle Antibodies (SMA), Antimitochondrial Antibodies (AMA), Liver Kidney Microsome Type 1 Antibodies (anti-LKM-1), Anti-Glomerular Basement Membrane (GBM), or Acetylcholine Receptor (aChR) Antibodies.

In various embodiments, the polypeptide B binds to an autoantibody that is associated with one or more autoimmune diseases including, but not limited to, Addison disease, Celiac disease such as sprue (gluten-sensitive enteropathy), dermatomyositis, Graves' disease, Hashimoto thyroiditis, multiple sclerosis, myasthenia gravis, pernicious anemia, reactive arthritis, Rheumatoid arthritis. Sjögren syndrome, systemic lupus erythematosus, and Type I diabetes, inflammatory bowel disease. Crohn's disease, Ulcerative colitis, psoriasis or psoriatic arthritis, and autoimmune vasculitis. In some embodiments, the autoimmune disease is associated with mucosal immunity or gut microbiota. Such diseases are described in Campbell A W. Autoimmunity and the gut. Autoimmune Diseases. 2014, Article ID 152428, which is incorporated herein by reference in its entirety.

### Pharmaceutical Compositions

In another embodiment, provided herein are pharmaceutical compositions comprising one or more conjugates disclosed herein and a pharmaceutically acceptable carrier.

In certain embodiments, the pharmaceutical compositions provided herein contain therapeutically effective amounts of one or more of the conjugates provided herein, and optionally one or more additional prophylactic or therapeutic agents, in a pharmaceutically acceptable carrier.

Pharmaceutical carriers suitable for administration of the conjugates provided herein include any such carriers known to those skilled in the art to be suitable for the particular mode of administration.

The conjugates described herein can be formulated as the sole pharmaceutically active ingredient in the composition or can be combined with other active ingredients.

In certain embodiments, the conjugate is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, a conjugate described herein may be mixed with a suitable pharmaceutical carrier. The concentration of the conjugate in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of conjugate is dissolved, suspended, dispersed or otherwise mixed in a selected carrier at an effective concentration such that the treated condition is relieved, prevented, or one or more symptoms are ameliorated.

Concentrations of the conjugate in a pharmaceutical composition provided herein will depend on, e.g., the physicochemical characteristics of the conjugate, the dosage schedule, and amount administered as well as other factors known to those of skill in the art.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a conjugate sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the conjugates herein are in a liquid pharmaceutical formulation. Liquid pharmaceutically administrable formulations can, for example, be prepared by dissolving, dispersing, or otherwise mixing a conjugate and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 2²ⁿd ed., Pharmaceutical Press, Philadelphia, PA Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutically carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a conjugate described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a conjugate provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the conjugates provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

### Uses and Methods

Binding of the ligand moiety of the bifunctional molecule to the ASGPR can trigger internalization and lysosomal degradation of a bound target autoantibody. In some embodiments, the bifunctional molecule is a conjugate of a protein that includes the polypeptide that specifically binds autoantibody and a linked ligand moiety. The bifunctional molecules of this disclosure find use in reducing levels of the extracellular target molecule autoantibody in a biological system or sample. The biological system can be a human subject.

The methods of using the conjugates described herein can thus remove autoantibodies from the extracellular space (the extracellular milieu) of a cell in the biological system by sequestering the target protein in the cell's lysosome and degrading the target autoantibodies. Removal of a target protein may refer to reduction of the amount of, or depletion of, the target protein from the extracellular space, or the extracellular milieu. In some embodiments, the biological system or sample is a cellular sample.

The term “sample” refers to an aliquot or portion taken from a source and/or provided for analysis or processing. In some embodiments, a sample is from a biological source such as a tissue, cell or component part (e.g., a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or includes a medium, such as a nutrient broth or gel, which may contain cellular components, such as proteins. In some embodiments, a “primary” sample is an aliquot of the source. In some embodiments, a primary sample is subjected to one or more processing (e.g., separation, purification, etc.) steps to prepare a sample for analysis or other use.

### Methods of Treating Disease

Provided herein are methods of treating a disease or disorder caused by the target autoantibody in a human subject. The present disclosure thus provides methods related to using the bifunctional molecules, conjugates and compositions of this disclosure for therapeutic treatment by depletion of patheogenic autoantibodies by degradation through the lysosomal pathway.

In some embodiments, the method of treating the disease includes administering to a subject, e.g., a human subject in need thereof, an effective amount of a bifunctional molecule or pharmaceutically acceptable salt thereof, or a pharmaceutical composition including the bifunctional molecule (e.g., as described herein).

In some embodiments, the patient treated according to the methods of this disclosure is a patient who is identified as being target autoantibody positive.

The terms “administer”, “administration”, or “administering” refer to the act of injecting or otherwise physically delivering a substance (e.g., a conjugate or pharmaceutical composition provided herein) to a subject or a patient (e.g., human), such as by mucosal, topical, intradermal, parenteral, intravenous, intramuscular delivery and/or any other method of physical delivery described herein or known in the art.

In some embodiments, administration is oral.

In some embodiments, administration is by subcutaneous injection.

In some embodiments, administration is by intravenous infusion.

The term “treating” refers to partially or completely alleviating, ameliorating, improving, relieving, delaying onset of, inhibiting progression of, reducing severity of, and/or reducing incidence of one or more symptoms or features of a particular disease, disorder, and/or condition (e.g., myasthenia gravis). Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and at least 20%, 30%, 40%, 50% or more can be indicative of effective treatment. Efficacy for a given compound or composition can also be judged using an experimental animal model for the given disease as known in the art. When using an experimental animal model, efficacy of treatment is evidenced when a statistically significant modulation in a marker or symptom is observed.

The terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result.

The term “treatment dose” refers to one or more doses of a therapeutic agent administered in the course of addressing or alleviating a therapeutic indication. Treatment doses may be adjusted to maintain a desired concentration or level of activity of a therapeutic agent in a body fluid or biological system.

A bifunctional molecule of this disclosure and additional therapeutic agent(s) and/or therapies for MG can be administered in combination. Such combinations may be in the same composition, or the additional therapeutic agents or therapies can be administered as part of a separate composition or by another method described herein.

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal. e.g., a human, diagnosed with a disease or disorder provided herein. In another embodiment, the subject is a mammal, e.g., a human, at risk of developing a disease or disorder provided herein. In some embodiments, the subject is human. The terms “patient”. “subject” and “individual” are used interchangeably herein. The term “screen” refers to a review or evaluation carried out for the purpose of selection or filtration. Patients may be screened to select individuals in need of treatment. In some embodiments, subjects are screened to select individuals most likely to respond favorably to treatment.

### Definitions

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. In certain embodiments, a polypeptide can occur as a single chain or as two or more associated chains, e.g., may be present as a multimer, e.g., dimer, a trimer. The terms also encompass an amino acid polymer that has been modified naturally or by intervention; for example, by disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. Polypeptides may contain L-amino acids. D-amino acids, or both and may contain any of a variety of amino acid modifications or analogs known in the art. Useful modifications include. e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%. 8%. 7%, 6%, 5%, 4%, 3%. 2%. 1%, 0.5%, 0.4%, 0.3%. 0.25%, 0.2%, 0.1% or 0.05% of a given value or range. In certain embodiments, where an integer is required, the term “about” means within plus or minus 10% of a given value or range, rounded either up or down to the nearest integer.

As used herein, the phrases “for example,” “for instance,” “such as.” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “autoantibody” or “autoantibodies” refers to an antibody that recognizes, binds, or otherwise interacts with an antigen normally found in a subject, or a tissue or cell of a subject. Autoantibodies are abnormal antibodies which are generated by pathogenic B cells when targeting an individual's own tissue. The term “autoimmunity” refers to the presence of antibodies (which are made by B lymphocytes) and T lymphocytes directed against normal components of a person (autoantigens). These components are called autoantigens or self-antigens and typically consist of proteins (or proteins complexed to nucleic acids). The antibodies and T lymphocytes that recognize autoantigens are called “autoantibodies” and “autoreactive T cells”.

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv fragments, and VHH fragments.

An “antigen” is a moiety or molecule that contains an epitope to which an antibody can specifically bind. As such, an antigen is also specifically bound by an antibody.

An “epitope” is a term known in the art and refers to a localized region of an antigen to which an antibody can specifically bind. An epitope can be a linear epitope of contiguous amino acids or can include amino acids from two or more non-contiguous regions of the antigen.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

A “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

The term “pharmaceutically acceptable salt” refers to those salts which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the conjugate compounds, or separately by reacting the free base function or group of a compound with a suitable organic acid.

Examples of pharmaceutically acceptable salts include, but are not limited to, nontoxic acid addition salts, or salts of an amino group formed with inorganic acids.

Compounds are described using standard nomenclature. The compounds in any of the formulas described herein may be in the form of a racemate, enantiomer, mixture of enantiomers, diastereomer, mixture of diastereomers, tautomer, N-oxide, isomer; such as rotamer, as if each is specifically described unless specifically excluded by context.

As used herein, the phrase “having the formula” or “having the structure” is not intended to be limiting and is used in the same way that the term “comprising” is commonly used. The term “independently selected from” is used herein to indicate that the recited elements. e.g., R groups or the like, can be identical or different.

A dash (“-”) that is not between two letters or symbols is used to indicate a point of attachment for a substituent. For example, —(C═O)NH₂ is attached through carbon of the carbonyl (C═O) group.

The present disclosure includes compounds (e.g., as described herein) with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C ¹⁴C N, ¹⁸F, ³¹P, ³²P, ³⁵S, ³⁶Cl, and ¹²⁵I respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in treatment of patients. In particular, an ¹⁸F labeled compound may be particularly desirable for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In some embodiments, the substitution of a hydrogen atom for a deuterium atom can be provided in any compound of Formulas described herein. In one non-limiting embodiment, the substitution of a hydrogen atom for a deuterium atom occurs within one or more groups selected from any of R¹, R², R³, R⁴, R⁶, R¹¹, R²¹, R²², R²³, R²⁴, R²⁵ R, R′, and R″ etc. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated.

“Aliphatic” refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In one embodiment, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In one embodiment, the aliphatic group contains from 1 to about 12 carbon atoms, more generally from 1 to about 6 carbon atoms or from 1 to about 4 carbon atoms. In one embodiment, the aliphatic group contains from 1 to about 8 carbon atoms. In certain embodiments, the aliphatic group is C₁-C₂, C₁-C₃, C₁-C₄, C₁-C₅ or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In one embodiment, the aliphatic group is substituted with one or more functional groups that results in the formation of a stable moiety.

“Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species and therefore each subset is considered separately disclosed. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In an alternative embodiment, the alkyl group is optionally substituted. The term “alkyl” also encompasses cycloalkyl or carbocyclic groups. For example, when a term is used that includes “alk” then “cycloalkyl” or “carbocyclic” can be considered part of the definition, unless unambiguously excluded by the context. For example and without limitation, the terms alkyl, alkoxy, haloalkyl, etc. can all be considered to include the cyclic forms of alkyl, unless unambiguously excluded by context.

“Alkenyl” is a linear or branched aliphatic hydrocarbon groups having one or more carbon-carbon double bonds that may occur at a stable point along the chain. The specified ranges as used herein indicate an alkenyl group having each member of the range described as an independent species, as described above for the alkyl moiety. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. In an alternative embodiment, the alkenyl group is optionally substituted. The term “Alkenyl” also encompasses cycloalkyl or carbocyclic groups possessing at least one point of unsaturation.

“Alkynyl” is a branched or straight chain aliphatic hydrocarbon group having one or more carbon-carbon triple bonds that may occur at any stable point along the chain. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl. 4-hexynyl and 5-hexynyl. In an alternative embodiment, the alkynyl group is optionally substituted. The term “Alkynyl” also encompasses cycloalkyl or carbocyclic groups possessing at least one triple bond.

“Alkylene” is a bivalent saturated hydrocarbon. Alkylenes, for example, can be a 1, 2, 3, 4, 5, 6, 7 to 8 carbon moiety, 1 to 6 carbon moiety, or an indicated number of carbon atoms, for example C₁-C₂alkylene, C₁-C₃alkylene, C₁-C₄alkylene, C₁-C₆alkylene, or C₁-C₆alkylene.

“Alkenylene” is a bivalent hydrocarbon having at least one carbon-carbon double bond. Alkenylenes, for example, can be a 2 to 8 carbon moiety, 2 to 6 carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, 2 to 6 carbon moiety, or an indicated number of carbon atoms, for example C₂-C₄alkynylene.

The term “amino” refers to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

“Chain” indicates a linear chain to which all other chains, long or short or both, may be regarded as being pendant. Where two or more chains could equally be considered to be the main chain, “chain” refers to the one which leads to the simplest representation of the molecule.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

“Halo” and “halogen” refers to fluorine, chlorine, bromine or iodine.

“Haloalkyl” is a branched or straight-chain alkyl groups substituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

“Haloalkoxy” indicates a haloalkyl group as defined herein attached through an oxygen bridge (oxygen of an alcohol radical).

The term “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In one embodiment, the only heteroatom is nitrogen. In one embodiment, the only heteroatom is oxygen. In one embodiment, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In one embodiment, “heteroaliphatic” is used to indicate a heteroaliphatic group (cyclic, acyclic, substituted, unsubstituted, branched or unbranched) having 1-20 carbon atoms. In one embodiment, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

“Heterocycloalkyl” is an alkyl group as defined herein substituted with a heterocyclo group as defined herein.

“Arylalkyl” is an alkyl group as defined herein substituted with an aryl group as defined herein.

“Heteroarylalkyl” is an alkyl group as defined herein substituted with a heteroaryl group as defined herein.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. The one or more fused carbocyclyl or heterocyclyl groups can be 4 to 7 or 5 to 7-membered saturated or partially unsaturated carbocyclyl or heterocyclyl groups that optionally contain 1, 2, or 3 heteroatoms independently selected from nitrogen, oxygen, phosphorus, sulfur, silicon and boron, to form, for example, a 3,4-methylenedioxyphenyl group. In one non-limiting embodiment, aryl groups are pendant. An example of a pendant ring is a phenyl group substituted with a phenyl group. In an alternative embodiment, the aryl group is optionally substituted as described above. In certain embodiments, the aryl group is an unsubstituted C₆₋₁₄ aryl. In certain embodiments, the aryl group is a substituted C₆₋₁₄ aryl. An aryl group may be optionally substituted with one or more functional groups that include but are not limited to, halo, hydroxy, nitro, amino, cyano, haloalkyl, aryl, heteroaryl, and heterocyclo.

The term “heterocyclyl” (or “heterocyclo”) includes saturated, and partially saturated heteroatom-containing ring radicals, where the heteroatoms may be selected from nitrogen, sulfur and oxygen. Heterocyclic rings comprise monocyclic 3-8 membered rings, as well as 5-16 membered bicyclic ring systems (which can include bridged fused and spiro-fused bicyclic ring systems). It does not include rings containing —O—O—, —O—S— or —S—S— portions. Said “heterocyclyl” group may be optionally substituted, for example, with 1, 2, 3, 4 or more substituents that include but are not limited to, hydroxyl, Boc, halo, haloalkyl, cyano, alkyl, aralkyl, oxo, alkoxy, and amino. Examples of saturated heterocyclo groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include but are not limited to, dihydrothienyl, dihydropyranyl, dihydrofuryl, and dihydrothiazolyl. Examples of partially saturated and saturated heterocyclo groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl.

Heterocyclo groups also include radicals where heterocyclic radicals are fused/condensed with aryl or heteroaryl radicals: such as unsaturated condensed heterocyclic group containing 1 to 5 nitrogen atoms, for example, indoline, isoindoline, unsaturated condensed heterocyclic group containing 1 to 2 oxygen atoms and 1 to 3 nitrogen atoms, unsaturated condensed heterocyclic group containing 1 to 2 sulfur atoms and 1 to 3 nitrogen atoms, and saturated, partially unsaturated and unsaturated condensed heterocyclic group containing 1 to 2 oxygen or sulfur atoms.

The term “heteroaryl” denotes aryl ring systems that contain one or more heteroatoms selected from O. N and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl. 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl. 1,2,5-thiadiazolyl].

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “optionally substituted” denotes the substitution of a group herein by a moiety including, but not limited to, C₁-C₁₀ alkyl, C₂-C₁₀ alkenyl, C₂-C₁₀ alkynyl, C₃-C₁₂ cycloalkyl, C₃-C₁₂cycloalkenyl, C₁-C₁₂heterocycloalkyl, C₃-C₁₂heterocycloalkenyl, C₁-C₁₀ alkoxy, aryl, aryloxy, heteroaryl, heteroaryloxy, amino, C₁-C₁₀ alkylamino, C₁-C₁₀ dialkylamino, arylamino, diarylamino, C₁-C₁₀alkylsulfonamino, arylsulfonamino, C₁-C₁₀ alkylimino, arylimino, C₁-C₁₀alkylsulfonimino, arylsulfonimino, hydroxyl, halo, thio, C₁-C₁₀ alkylthio, C₁-C₁₀alkylsulfonyl, arylsulfonyl, acylamino, aminoacyl, aminothioacyl, amidino, guanidine, ureido, cyano, nitro, azido, acyl, thioacyl, acyloxy, carboxyl, and carboxylic ester.

In one alternative embodiment any suitable group may be present on a “substituted” or “optionally substituted” position if indicated that forms a stable molecule and meets the desired purpose of the invention and includes, but is not limited to, e.g., halogen (which can independently be F, Cl, Br or I); cyano; hydroxyl; nitro; azido; alkanoyl (such as a C₂-C₆alkanoyl group); carboxamide; alkyl, cycloalkyl, alkenyl, alkynyl, alkoxy, aryloxy such as phenoxy; thioalkyl including those having one or more thioether linkages; alkylsulfinyl; alkylsulfonyl groups including those having one or more sulfonyl linkages; aminoalkyl groups including groups having more than one N atoms; aryl (e.g., phenyl, biphenyl, naphthyl, or the like, each ring either substituted or unsubstituted); arylalkyl having for example, 1 to 3 separate or fused rings and from 6 to about 14 or 18 ring carbon atoms, with benzyl being an exemplary arylalkyl group; arylalkoxy, for example, having 1 to 3 separate or fused rings with benzyloxy being an exemplary arylalkoxy group; or a saturated or partially unsaturated heterocycle having 1 to 3 separate or fused rings with one or more N. O or S atoms, or a heteroaryl having 1 to 3 separate or fused rings with one or more N, O or S atoms. e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such groups may be further substituted, e.g. with hydroxy, alkyl, alkoxy, halogen and amino. In certain embodiments “optionally substituted” includes one or more substituents independently selected from halogen, hydroxyl, amino, cyano, —CHO, —COOH, —CONH₂, alkyl including C₁-C₆alkyl, alkenyl including C₂-C₆alkenyl, alkynyl including C₂-C₆alkynyl, —C₁-C₆alkoxy, alkanoyl including C₂-C₆alkanoyl, C₁-C₆alkylester, (mono- and di-C₁-C₆alkylamino)C₀-C₂alkyl, haloalkyl including C₁-C₆haloalkyl, hydoxyC₁-C₆alkyl, ester, carbamate, urea, sulfonamide, —C₁-C₆alkyl(heterocyclo), C₁-C₆alkyl(heteroaryl), —C₁-C₆alkyl(C₃-C₇cycloalkyl), O—C₁-C₆alkyl(C₃-C₇cycloalkyl), B(OH)₂, phosphate, phosphonate and haloalkoxy including C₁-C₆haloalkoxy.

When the term “substituted” appears prior or after a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined herein.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “hydroxyalkyl” refers to the group HO-(alkyl)-.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound.

A compound of this disclosure may form a solvate with a solvent (including water). Therefore, in one non-limiting embodiment, the present disclosure includes a solvated form of the compound. The term “solvate” refers to a molecular complex of a compound (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound and water. Pharmaceutically acceptable solvates in accordance with the invention include those wherein the solvent may be isotopically substituted, e.g. D20, d6-acetone, d6-DMSO. A solvate can be in a liquid or solid form.

Salts, solvates, hydrates, and prodrug forms of a compound are of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Definitions of other terms and concepts appear throughout the detailed description.

### Additional Embodiments

Aspects of the present disclosure are described in the following clauses.

Clause 1: An extracellular target binding conjugate of formula (I):

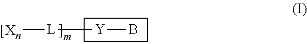

or a pharmaceutically acceptable salt thereof, wherein:
X is a moiety that binds to a lysosomal targeting molecule;
n is 1 to 50 (e.g., 1 to 40, 1 to 30, 1 to 20, or 1 to 10, 1 to 6, or 1 to 4, such as 1, 2, or 3);
L is a linker;
m is the average number of (Xₙ-L) moieties conjugated to Y—B, wherein m is in the range from about 1 to about 20 (e.g., about 1 to about 3, such as 1, 2 or 3, or about 1 to about 10, about 1 to about 8, about 2 to about 8, about 3 to about 6, or about 4 to about 5);
Y is an optional carrier polypeptide connected to B; and
B is a polypeptide that specifically binds an extracellular target molecule.

Clause 2: The conjugate of clause 1, wherein B specifically binds a target antibody (e.g., an extracellular antibody associated with a disease or condition of interest).

Clause 3: The conjugate of clause 2, wherein the target antibody is an autoantibody.

Clause 4: The conjugate of clause 2, wherein the target antibody is a neutralizing antibody or an anti-drug antibody.

Clause 5: The conjugate of any one of clauses 2 to 4 wherein B comprises an antigen of the target antibody, or fragment thereof (e.g., synthetic peptides).

Clause 6: The conjugate of any one of clauses 1 to 5, wherein B comprises a protein domain.

Clause 7: The conjugate of any one of clauses 1 to 6, wherein Y comprises a protein domain.

Clause 8: The conjugate of any one of clauses 1 to 7, wherein Y is fused directly to B.

Clause 9: The conjugate of any one of clauses 1 to 7, wherein Y is fused indirectly to B via a spacer domain.

Clause 10: The conjugate of any one of clauses 1 to 9, wherein Y—B is a chimeric protein.

Clause 11: The conjugate of any one of clauses 1 to 10, wherein Y is selected from human serum albumin (HSA), HSA domain, albumin binding domain, Fc (monomer). Fc (dimer), and fragments thereof (e.g., synthetic peptides).

Clause 12: The conjugate of clause 10 or 11, wherein Y—B is selected from an Fc-VHH antigen fusion, an Fc-antigen fusion, and an HSA-antigen fusion.

Clause 13: The conjugate of any one of clauses 1 to 6, wherein Y is covalently linked to B via a linker.

Clause 14: The conjugate of clause 13, wherein the linker comprises a non-peptidic linking moiety.

Clause 15: The conjugate of any one of clauses 13 to 14, wherein Y is selected from human serum albumin (HSA), HSA domain, albumin binding domain, Fc (monomer). Fc (dimer), and fragments thereof (e.g., synthetic peptides).

Clause 16: The conjugate of any one of clauses 1 to 15, wherein m is 1 to 10 (e.g., 1 to 6, or 1 to 4, where m can be a discrete loading or an average loading (i.e. DAR ratio)).

Clause 17: The conjugate of clause 16, wherein m is 1 to 2.

Clause 18: The conjugate of clause 17, wherein m is 1.

Clause 19: The conjugate of clause 17, wherein m is 2.

Clause 20: The conjugate of any one of clauses 16, wherein m is 2 to 4.

Clause 21: The conjugate of clause 20, wherein m is 3.

Clause 22: The conjugate of clause 20, wherein m is 4.

Clause 23: The conjugate of any one of clauses 1 to 22, wherein n is 1 to 20 (e.g., 1 to 10, 1 to 6, or 1 to 4 for M6PR ligands, or e.g., 1 to 3 for GalNAc ligands).

Clause 24: The conjugate of clause 23, wherein n is 1 to 2.

Clause 25: The conjugate of clause 24, wherein n is 1.

Clause 26: The conjugate of clause 24, wherein n is 2.

Clause 27: The conjugate of clause 23, wherein n is 3.

Clause 28: The conjugate of any one of clauses 1 to 27, wherein n is 1, and L comprises a linear linker having a backbone of 20 or more consecutive atoms covalently linking X to Y—B.

Clause 29: The conjugate of any one of clauses 1 to 27, wherein n is 2, and L is a branched linker that covalently links the X moieties to Y—B.

Clause 30: The conjugate of any one of clauses 1 to 27, wherein n is 3, and L is a branched linker that covalently links the X moieties to Y—B.

Clause 31: The conjugate of any one of clauses 1 to 30, wherein the linker L comprises a backbone of 20 to 60 consecutive atoms between each X and Y—B (e.g., 20 to 50, 20 to 40, 30 to 40, 30 to 60, or 40 to 60 consecutive atoms).

Clause 32: The conjugate of any one of clauses 1 to 31, wherein L is of formula (XI):

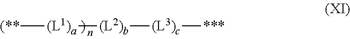

wherein
L¹ and L³ are independently a linear linking moiety, and L² is a branched linking moiety, wherein L¹ to L³ together provide a linear or branched linker between X and Y;
a, b and c are independently 0 or 1;
** represents the point of attachment to L¹ of X via Z¹; and
** represents the point of conjugation of the linker L to Y—B.

Clause 33: The conjugate of clause 32, wherein:
n is 1; and
a is 1, b is 0 and c is 1, whereby L is of formula (Xa):

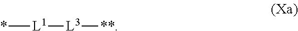

Clause 34: The conjugate of clause 32, wherein:
n is 2; and
a is 1, b is 1, and c is 1, whereby L is of formula (Xb):

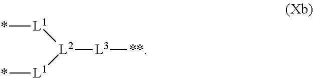

Clause 35: The conjugate of clause 32, wherein:
n is 3; and
a is 1, b is 1, and c is 1, whereby L is of formula (Xc):

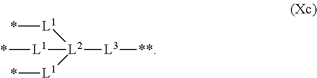

Clause 36: The conjugate of any one of clauses 32 to 35, wherein each L¹ is of the formula (XII)

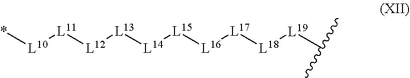

wherein:
L¹⁰ is a linking moiety; and
L¹¹ to L¹⁹ are independently absent or a linking moiety,
wherein each linking moiety of each L¹ is independently selected from —C₁₋₂₀-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ, —NHCO—, —CONH—, —NHSO₂—, —SO₂NH—, —CO—, —SO₂—, —O—, —S—, pyrrolidine-2,5-dione, 1,2,3-triazole, —NH—, and -nMe-, wherein each p is independently 1 to 50.

Clause 37: The conjugate of any one of clauses 32 to 36, wherein each L¹ comprises a linear backbone of 6 to 20 consecutive atoms (e.g., 6 to 16 consecutive atoms, such as 8, 9, 10, 11, 12, 13, 14, 15 or 16 consecutive atoms).

Clause 38: The conjugate of any one of clauses 32, and 34 to 37, wherein L² is of formula (XIIIa) or (XIIIb):

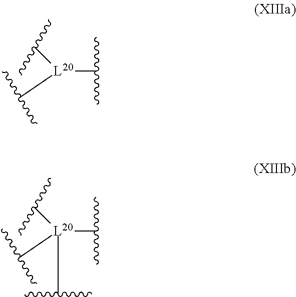

wherein:
L²⁰ is a branched linking moiety comprising one or more linking moieties independently selected from amino acid residue (e.g., a residue such as Gly, -Ala, Lys, Orn, Asp, Glu, Ser, Cys, or a derivative thereof), —NH—CH[(CH₂)_{q}]₂O— or —NH—C[(CH₂)_{q}]₃O—

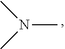

—C₁₋₆-alkylene-, —NHCO—, —CONH—, —NHSO₂—, —SO₂NH—, —CO—, —SO₂—, —O—, —S—, pyrrolidine-2,5-dione, 1,2,3-triazole, —NH—, and -nMe-, —NHC(═O)NH—, —NHC(═S)NH—, —O(CH₂)ₚ—, and —(OCH₂CH₂)ₚ—;
wherein each p is independently 1 to 50, and q is 1-6.

Clause 39: The conjugate of any one of clauses 32, and 34 to 38, wherein L² comprises a linking moiety selected from:

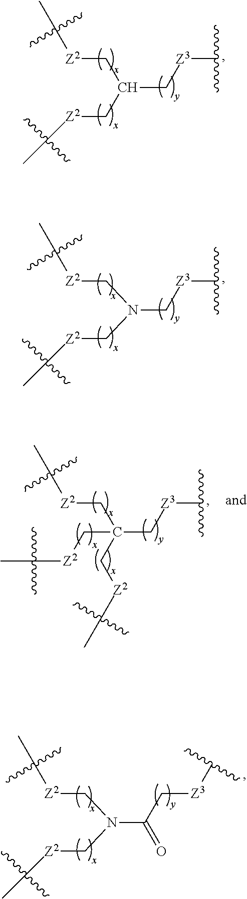

wherein:
each Z² and Z³ is independently selected from —NHCO—, —CONH—, —CO—, —O—, —NH—, and -nMe-;
x is 1 to 12 (e.g., 1 to 6, or 1 to 3); and
y is 0 to 12 (e.g., 1 to 6, or 1 to 3).

Clause 40: The conjugate of clause 39, wherein L² comprises a linking moiety selected from:

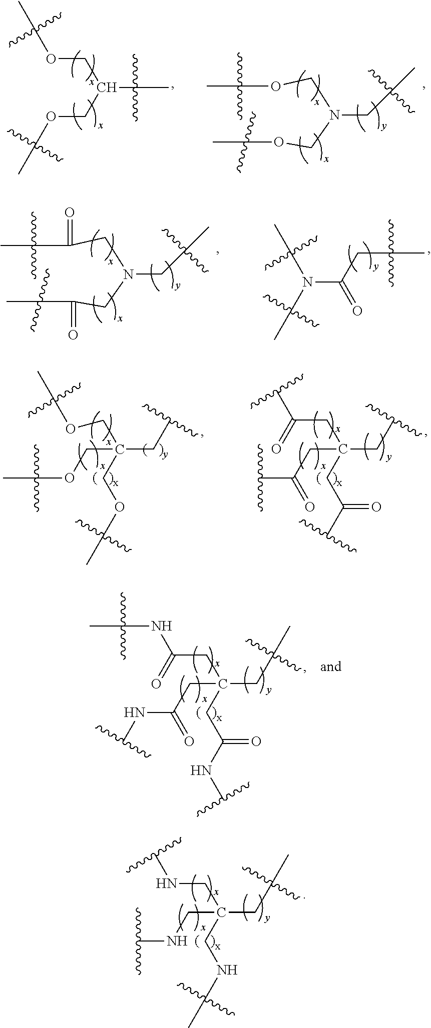

Clause 41: The conjugate of clause 40, wherein L² comprises a linking moiety of formula (XIV):

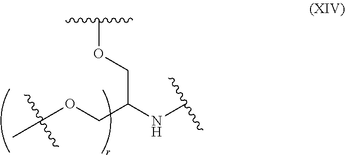

wherein:
r is 1 or 2; and
when n is 2, r is 1, and
when n is 3, r is 2.

Clause 42: The conjugate of clause 41, wherein L² is of formula (XVa) or (XVb)):

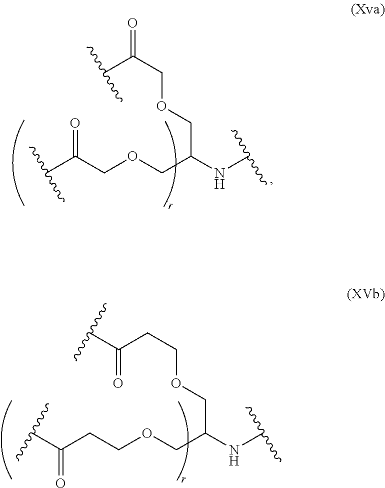

Clause 43: The conjugate of clause 38, wherein L² comprises two 2 or more amino acid residues (e.g., 3 or more, or 4 or more amino acid residues, linear or dendrimer).

Clause 44: The conjugate of clause 43, wherein L² comprises 4 or more amino acid residues that are branched linking moieties selected from Lys, Orn, Asp, Glu, Ser, and Cys (e.g., where the sidechain, amino and carboxylic acid are each linked to an adjacent moiety).

Clause 45: The conjugate of any one of clauses 32 to 44, wherein each L³ is of the formula (XVI):

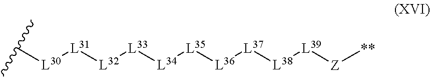

wherein:
L³⁰ to L³⁹ are independently absent or a linking moiety; and
Z is a residual moiety resulting from the covalent linkage of a chemoselective ligation group of the linker to a compatible group of Y—B;
wherein each linking moiety of L³ is independently selected from —C₁₋₂₀-alkylene-, —NHCO—C₁₋₆-alkylene-, —CONH—C₁₋₆-alkylene-, —NHC₁₋₆-alkylene-, —NHCONH—C₁₋₆-alkylene-, —NHCSNH—C₁₋₆-alkylene-, —C₁₋₆-alkylene-NHCO—, —C₁₋₆-alkylene-CONH—, —C₁₋₆-alkylene-NH—, —C₁₋₆-alkylene-NHCONH—, —C₁₋₆-alkylene-NHCSNH—, —O(CH₂)ₚ—, —(OCH₂CH₂)ₚ—, —NHCO—, —CONH—, —NHSO₂—, —SO₂NH—, —CO—, —SO₂—, —O—, —S—, pyrrolidine-2,5-dione, 1,2,3-triazole, —NH—, and -nMe-, wherein each p is independently 1 to 50.

Clause 46: The conjugate of clause 45, wherein L³ comprises a linear backbone of 6 to 40 consecutive atoms (e.g., 10 to 30 consecutive atoms, or 20 to 30 consecutive atoms).

Clause 47: The conjugate of clause 45 or 46, wherein the linker L has the following structure:

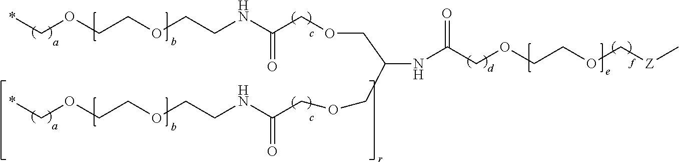

wherein:
a is 1 to 12 (e.g., 2 to 6, or 2, or 3);
b is 1 to 6 (e.g., 1, 2, or 3);
c is 1 to 6 (e.g., 1, 2, or 3);
r is 1 or 2;
d is 1 to 6 (e.g., 1, 2, or 3);
e is b is 1 to 6 (e.g., 1, 2, or 3);
f is 1 to 6 (e.g., 2, or 3);
Z is a residual moiety resulting from the covalent linkage of a chemoselective ligation group of the linker to a compatible group of Y—B.

Clause 48: The conjugate of clause 47, wherein the linker L comprises one of the following structures:

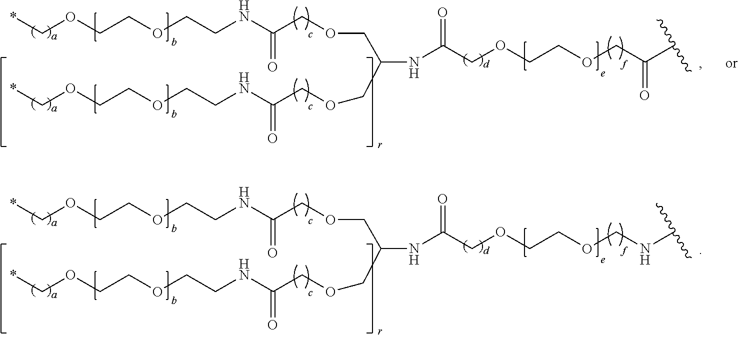

Clause 49: The conjugate of any one of clauses 32 to 45, wherein the conjugate is of formula (Ia):

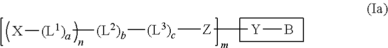

wherein:
Z is residual moiety resulting from the covalent linkage of a chemoselective ligation group of the linker to a compatible group of Y—B;
n is 1, 2, or 3; and
m is the average number of (Xₙ-L) moieties conjugated to Y—B, wherein m is in the range from about 1 to about 8.

Clause 50: The conjugate of clause 49, wherein Z is a residual moiety resulting from the covalent linkage of a thiol-reactive chemoselective ligation group to one or more cysteine residue(s) of Y—B.

Clause 51: The conjugate of clause 50, wherein the thiol-reactive chemoselective ligation group comprises maleimide, bromomaleimide, haloacetamide, vinyl sulfone, or thiolactone.

Clause 52: The conjugate of clause 51, wherein the thiol-reactive chemoselective ligation group is selected from one of the following structures:

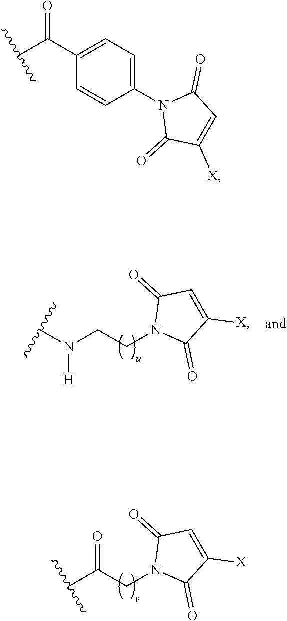

wherein:
u is 1 to 11 (e.g., 1 to 5);
v is 1 to 11 (e.g., 1 to 5); and
X is H or Br.

Clause 53: The conjugate of any one of clauses 50 to 52, wherein m is the average number of (Xₙ-L) moieties conjugated to Y—B, wherein m corresponds to the number of solvent accessible cysteine residues in Y—B.

Clause 54: The conjugate of clause 53, wherein m is about 3 or less.

Clause 55: The conjugate of clause 54, wherein m is about 1.

Clause 56: The conjugate of clause 54, wherein m is about 2.

Clause 57: The conjugate of clause 54, wherein n is about 3.

Clause 58: The conjugate of clause 49, wherein Z is a residual moiety resulting from the covalent linkage (e.g., via an amide bond) of an amine-reactive chemoselective ligation group to one or more lysine residue(s) of Y—B.

Clause 59: The conjugate of clause 58, wherein the amine-reactive chemoselective ligation group comprises an active ester (e.g., N-hydroxysuccinimidyl (NHS) ester, sulfo-NHS ester, pentafluorophenyl (PFP) ester, tetrafluorophenyl (TFP) ester, or the like).

Clause 60: The conjugate of clause 58 or 59, wherein in is the average number of (Xₙ-L) moieties conjugated to Y—B, wherein in is about 3 to about 6.

Clause 61: The conjugate of clause 60, wherein m is about 4 to about 5.

Clause 62: The conjugate of any one of clauses 1 to 61, wherein n is 1.

Clause 63: The conjugate of any one of clauses 1 to 61, wherein n is 2.

Clause 64: The conjugate of any one of clauses 1 to 61, wherein n is 3.

Clause 65: The conjugate of any one of clauses 1 to 64, wherein the lysosomal targeting molecule is selected from asialoglycoprotein receptor (ASGPR), M6PR, folate receptor, CD63, transferrin, sortilin, IFITM3, molecules in the endosome/lysosome pathway, LIMP-1, and LIMP-2.

Clause 66: The conjugate of any one of clauses 1 to 65, wherein X is a moiety that binds to a cell surface asialoglycoprotein receptor (ASGPR).

Clause 67: The conjugate of clause 65, wherein X is of formula (II):

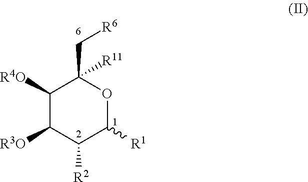

wherein:
R¹ is selected from —Z¹—*, —H, —OH, —CH₃, —OCH₃, and —OCH₂CH═CH;
R² is selected from —Z¹—*, —NHCOCH₃, —NHCOCF₃, —NHCOCH₂CF₃, —OH, and optionally substituted triazole;
R⁶ is selected from —Z¹—*, —OH, —OC(O)R, —C(O)NHR, and optionally substituted triazole, where R is optionally substituted (C₁-C₆)alkyl or optionally substituted aryl;
wherein one of R¹, R², and R⁶ is —Z¹—*, and “*” represents a point of connection of Z¹ to the linker (L):
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring;
Z¹ is a linking moiety selected from —Z¹¹—, —Z¹¹-A¹-, -A²-, —NR²¹CO—, —CONR²¹—, —NR²¹SO₂—, —SO₂NR²¹—, —NR²¹C(═O)NR²¹—, and —NR²¹C(═S)NR²¹—;
—Z¹¹— is —O—, —S—, —N(R²¹)—, or —C(R²²)₂:
-A¹- and -A²- are optionally substituted arylene or optionally substituted heteroarylene;
each R²¹ is independently selected from H, and optionally substituted (C₁-C₆)alkyl; and
each R²² is independently selected from H, halogen (e.g., F) and optionally substituted (C₁-C₆)alkyl.

Clause 68: The conjugate of clause 67, wherein R¹ is —Z¹—* and each X is independently of formula (IIa):

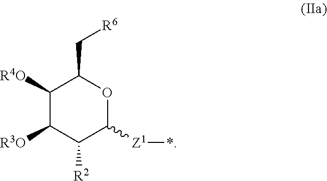

Clause 69: The conjugate of clause 68, wherein each X is independently of formula (IVa) or (IVb):

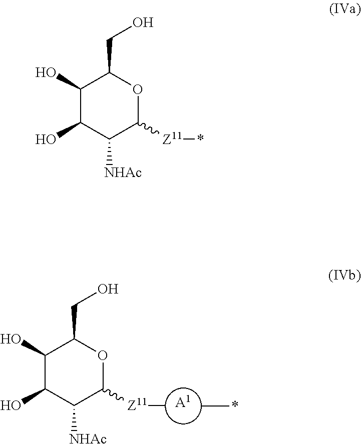

wherein:
—Z¹¹— is —O—, —S—, —N(R²¹)—, or —C(R²²)₂; and
-A¹- is arylene, substituted arylene, heteroarylene, or substituted heteroarylene.

Clause 70: The conjugate of clause 69, wherein Z¹¹ is —S—.

Clause 71: The conjugate of clause 68 or 69, wherein each X is independently of formula (iIa-1):

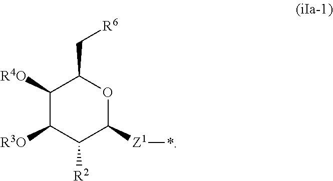

Clause 72: The conjugate of clause 71, wherein Z¹ is S, and each X is independently of formula (X^{A}):

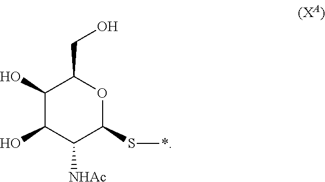

Clause 73: The conjugate of clause 68 or 69, wherein each X is independently of formula (IIa-2):

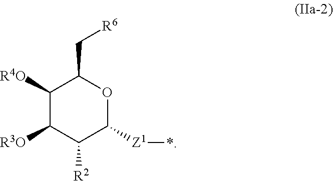

Clause 74: The conjugate of clause 73, wherein Z¹ is S, and each X is independently of formula (X^{B}):

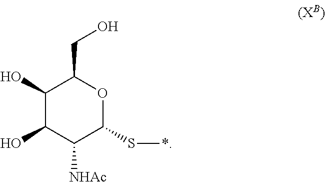

Clause 75: The conjugate of clause 68, 69, 71 or 73, wherein Z¹¹ is —C(R²²)₂.

Clause 76: The conjugate of clause 75, wherein each X is independently of formula (X^{C}):

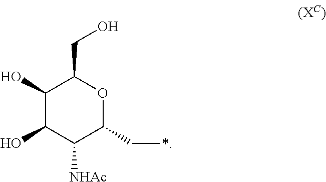

Clause 77: The conjugate of clause 75, wherein each X is independently of formula (IIIf):

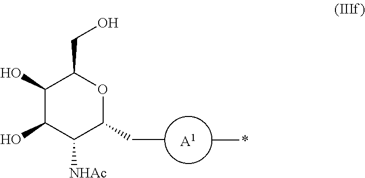

wherein:
-A¹- is arylene, substituted arylene, heteroarylene, or substituted heteroarylene.

Clause 78: The conjugate of clause 77, wherein each X is independently of formula (X^{C}) or (X^{D}-2):

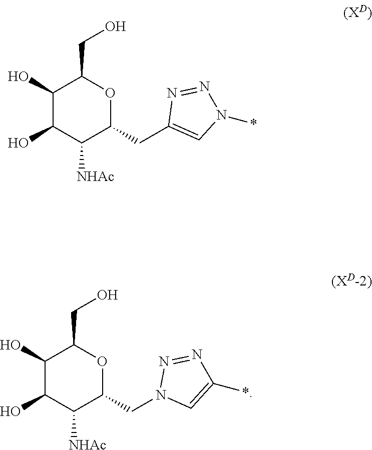

Clause 79: The conjugate of clause 67, wherein R² is —Z¹—* and each X is independently of formula (IIb):

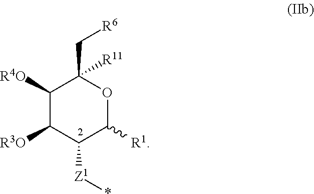

Clause 80: The conjugate of clause 79, wherein each X is independently of formula (IVa):

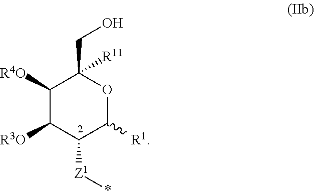

Clause 81: The conjugate of clause 80, wherein each X is independently of formula (IVb) or (IVc):

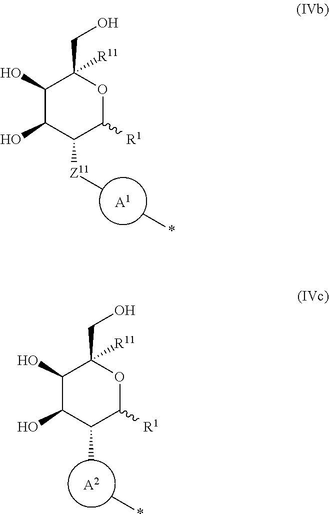

wherein —Z¹¹— is —O—, —S—, —N(R²¹)—, or —C(R²²)₂.

Clause 82: The conjugate of clause 80 or 81, wherein R¹ is H.

Clause 83: The conjugate of any one of clauses 80 to 82, wherein R¹¹ is H.

Clause 84: The conjugate of clause 80 or 81, wherein each X is independently of formula (IVb-1) or (IVc-1):

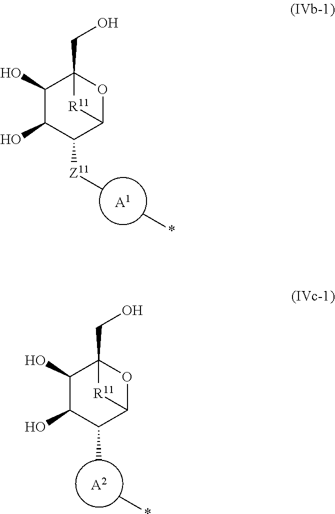

wherein R¹¹ is the bridging moiety that connects the 5-position carbon to the 1-position carbon.

Clause 85: The conjugate of clause 84, wherein R¹¹ is —CH₂O— or —OCH₂—.

Clause 86: The conjugate of any one of clauses 81 to 85, wherein: —Z¹¹— is —N(R²¹)—; and
-A¹- and -A²- are optionally substituted monocyclic heteroarylene (e.g., triazole or pyrimidine).

Clause 87: The conjugate of clause 86, wherein each X is independently of formula (X^{E}-1) or (X^{F}):

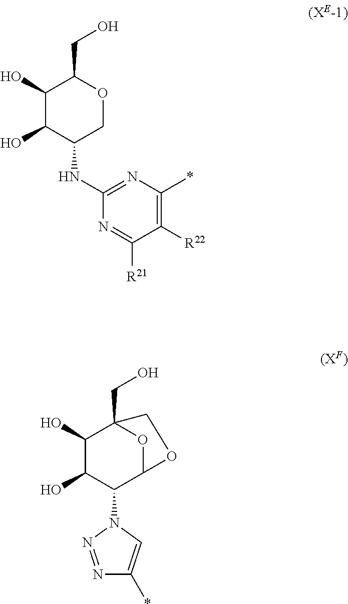

wherein R²¹ and R²² are independently selected from H, halogen, (C₁₋₆)alkyl and substituted (C₁-C₆)alkyl (e.g., CF₃).

Clause 88: The conjugate of clause 67, wherein R⁶ is —Z¹—* and each X is independently of formula (IIc):

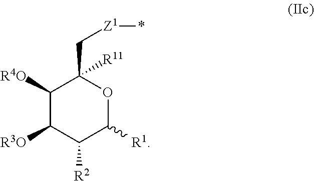

Clause 89: The conjugate of any one of clauses 1 to 88, wherein the conjugate is derived from a ligand linker compound of Table 1, wherein:
X is:

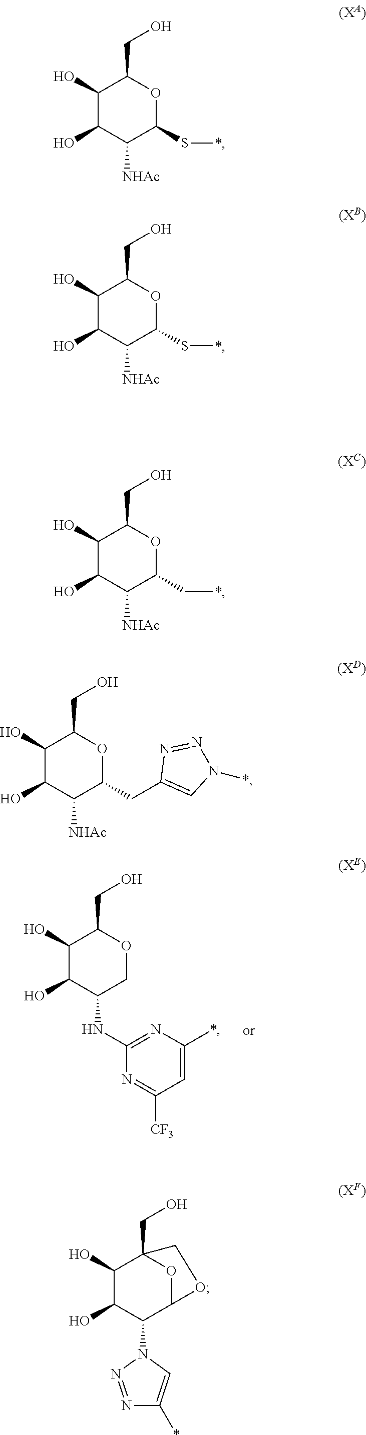

L is:

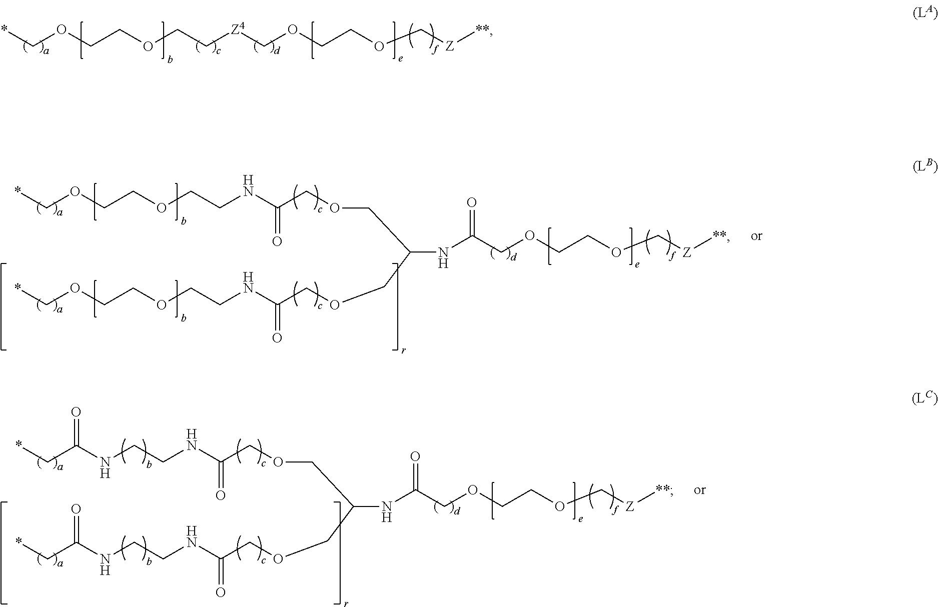

and
Z is derived from a Z precursor that is:

| | |
|---|---|
| | Z^{A} |
| 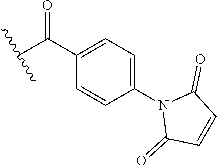 [SMILES: *C(=O)c1ccc(N2C(=O)C=CC2=O)cc1] | |
| | Z^{B} |
| 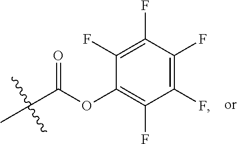 [SMILES: *C(=O)Oc1c(F)c(F)c(F)c(F)c1F ; "or"] | |
| | Z^{C} |
| 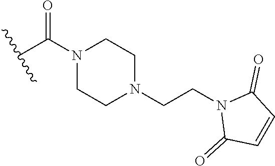 [SMILES: *C(=O)N1CCN(CCN2C(=O)C=CC2=O)CC1] | |

| Cmpd | | | | | | | | | | | Z |
|---|---|---|---|---|---|---|---|---|---|---|---|
| # | X | L | a | b | c | r | d | e | f | Z⁴ | precursor |
| 1 | X^{C} | L^{B} | 2 | 1 | 2 | 1 | 2 | 3 | 2 | — | Z^{B} |
| 2 | X^{C} | L^{B} | 2 | 1 | 2 | 2 | 2 | 3 | 2 | — | Z^{B} |
| 3 | X^{C} | L^{B} | 2 | 1 | 2 | 2 | 2 | 3 | 2 | — | Z^{C} |
| 4 | X^{C} | L^{B} | 2 | 1 | 2 | 1 | 2 | 3 | 2 | — | Z^{C} |
| 5 | X^{C} | L^{A} | 4 | 1 | 2 | — | 2 | 5 | 2 | —NHC(O)NH— | Z^{B} |
| 6 | X^{C} | L^{B} | 4 | 1 | 2 | 2 | 2 | 3 | 2 | — | Z^{A} |
| 7 | X^{C} | L^{B} | 4 | 1 | 2 | 1 | 2 | 3 | 2 | — | Z^{A} |
| 8 | X^{C} | L^{B} | 2 | 1 | 2 | 2 | 2 | 3 | 2 | — | Z^{A} |
| 9 | X^{D} | L^{B} | 2 | 2 | 2 | 2 | 2 | 3 | 2 | — | Z^{A} |
| 10 | X^{D} | L^{B} | 2 | 2 | 2 | 1 | 2 | 3 | 2 | — | Z^{A} |
| 11 | X^{B} | L^{B} | 2 | 2 | 2 | 2 | 2 | 3 | 2 | — | Z^{A} |
| 12 | X^{E} | L^{B} | 0 | 3 | 2 | 2 | 2 | 3 | 2 | — | Z^{A} |
| 13 | X^{F} | L^{B} | 1 | 2 | 2 | 2 | 2 | 3 | 2 | — | Z^{A} |
| 14 | X^{A} | L^{C} | 2 | 4 | 2 | 2 | 2 | 3 | 2 | — | Z^{A} |
| 15 | X^{B} | L^{C} | 2 | 4 | 2 | 2 | 2 | 3 | 2 | — | Z^{A}. |

Clause 90: The conjugate of any one of clauses 1 to 64, wherein X is a moiety that binds to a cell surface mannose-6-phosphate receptor (M6PR).

Clause 91: The conjugate of clause 90, wherein X is of formula (IV):

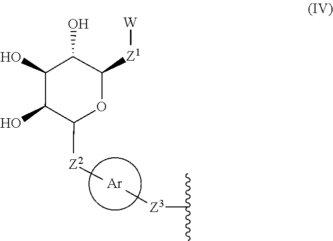

wherein:
W is a non-hydrolyzable hydrophilic head group;
Z¹ is selected from optionally substituted (C₁-C₃)alkylene and optionally substituted ethenylene;
Z² is selected from O. S, NR²¹ and C(R²²)₂, wherein each R²¹ is independently selected from H, and optionally substituted (C₁-C₆)alkyl, and each R²² is independently selected from H, halogen and optionally substituted (C₁-C₆)alkyl;
each A is independently an optionally substituted aryl or heteroaryl linking moiety; and
each Z³ is independently a linking moiety.

Clause 92: A method of reducing levels of an extracellular target molecule in a biological system, the method comprising:
contacting the biological system with an effective amount of a conjugate according to any one of clauses 1 to 91, wherein the compound specifically binds the extracellular target molecule and specifically binds a lysosomal targeting molecule of cells in the biological system to facilitate cellular uptake and degradation of the extracellular target molecule.

Clause 93: The conjugate of clause 12, wherein Y—B is an HSA-antigen fusion.

Clause 94: The conjugate of 15, wherein Y is HSA.

Clause 95: The method of clause 92 wherein the target molecule is an autoantibody.

Clause 96: The method of any of clauses 92-95 wherein the target molecule is a TSH receptor autoantibody.

Clause 97: The method of any of clauses 92-95 wherein the target molecule is a MuSK antibody.

Clause 98: The method of any of clauses 92-95 wherein the target molecule is an aChR antibody.

Clause 99: The method of any of clauses 92-95 wherein the target molecule is a BP180 antibody.

Clause 100: The method of any of clauses 92-95 wherein the target molecule is a PR3 antibody.

Clause 101: The method of any of clauses 92-95 wherein the lysosomal targeting molecule is ASGPR.

Clause 102: The method of any of clauses 92-95 wherein the lysosomal targeting molecule is M6PR.

### EXAMPLES

The examples in this section are offered by way of illustration, and not by way of limitation.

### Preparation of Compounds

The following are illustrative schemes and examples of how the compounds described herein can be prepared and tested. Although the examples can represent only some embodiments, it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

### Synthesis of Compound 1209

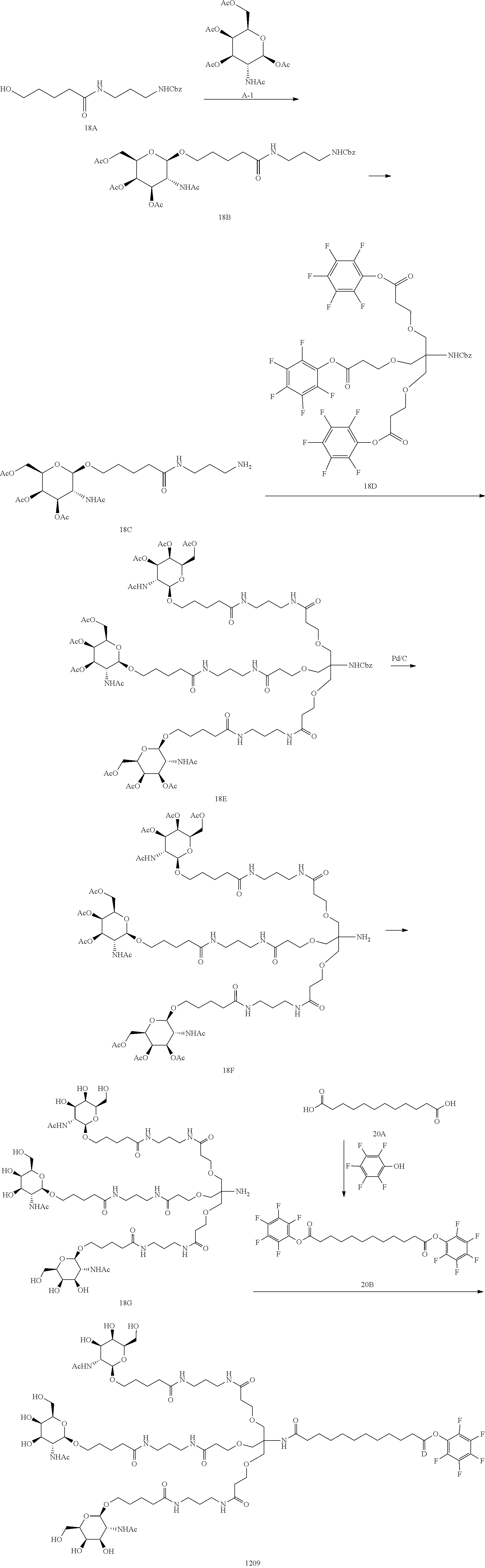

To the solution of Compound A-1 (1.0 eq, 5.05 g, 13.0 mmol) and benzyl N-[3-(5-hydroxypentanamido) propyl]carbamate (Compound 18A) (1.0 eq, 4.00 g, 13.0 mmol) in dichloromethane (50.0 mL), trimethylsilyl trifluoromethanesulfonate (1.1 eq, 2.52 mL, 14.3 mmol) was added dropwise at room temperature. The reaction mixture was stirred at 40° C. for 5 h. After completion, the reaction mixture was quenched with saturated sodium bicarbonate solution and extracted with dichloromethane. The organic layer was dried over sodium sulfate, filtered, and concentrated under high vacuum to get crude. The crude was purified by reverse phase chromatography using 0-30% acetonitrile in water to afford Compound 18B as yellow viscous liquid, Yield: (5.80 g, 70.12%); LCMS m/z 638.2 [M+1]⁻

To a solution of Compound 18B (1.0 eq, 4.80 g, 7.53 mmol) in methanol (40.0 mL), 10% palladium on carbon (1.60 g) was added and stirred at room temperature under hydrogen atmosphere for 4 h. After completion, the reaction mixture was filtered through syringe filter, filtrate was concentrated and dried to get crude. The crude was triturated with diethyl ether to afford Compound 18C as a pale yellow viscous liquid. Yield: (3.4 g, 80.73%); LCMS m/z 504.37 [M+1]⁺.

A solution of 2,3,4,5,6-pentafluorophenyl 3-(2-{[(benzyloxy)carbonyl]amino}-3-[3-oxo-3-(2,3,4,5,6-pentafluorophenoxy)propoxy]-2-{[3-oxo-3-(2,3,4,5,6-pentafluorophenoxy)propoxy]methyl}propoxy)propanoate (18D) (1.0 eq, 1.20 g, 1.24 mmol) and Compound 18C (3.0 eq, 1.87 g, 3.71 mmol) in N,N-dimethylformamide (30.0 mL) was stirred at room temperature for 1 h. After completion, the reaction mixture was concentrated and dried to get crude. The crude was purified by flash column chromatography using 20% methanol in dichloromethane to afford Compound 18E as pale yellow viscous liquid. Yield: (1.60 g; 67.05%); LCMS m/z 1926.78 [M−1]⁻.

To a solution of Compound 18E (1.0 eq, 1.60 g, 0.830 mmol) in methanol (20 mL) and acetic acid (1.0 mL), 10% palladium on carbon (250 mg) was added. The reaction mixture was stirred at room temperature under hydrogen atmosphere for 16 h. After completion, the reaction mixture was filtered through celite bed, filtrate was concentrated and dried to afford Compound 18F as pale yellow viscous liquid. Yield: 1.45 g (Crude); LCMS m/z 1794.05 [M+1]⁺.

To a solution of Compound 18F (1.0 eq. 1.45 g, 0.808 mmol) in methanol (10 mL), 25% sodium methanolate solution (8.0 eq, 1.45 mL, 6.47 mmol) was added at 0° C. The reaction mixture was stirred at room temperature for 1 h. After completion reaction, reaction mixture was concentrated and dry to get crude. The crude was diluted with acetonitrile and purified by preparative HPLC (30% acetonitrile in water with 0.1% TFA). Fractions containing the desired product were combined and lyophilized to dryness to afford Compound 18G as an off white semi solid. Yield: (0.20 g, 17.4%); LCMS m/z 1415.77 [M+1]⁺.

To the solution of dodecanedioic acid (20A) (1.00 g, 4.34 mmol) in ethyl acetate (10.00 mL) at 0° C., pentafluorophenol (1.60 g, 8.68 mmol) and diisopropylnethanediimine (1.91 mL, 13.0 mmol) were added and reaction mixture stirred at room temperature for 1 h. After completion, the reaction mixture was filtered through celite bed and filtrate was concentrated under reduced pressure to get crude compound. Crude compound obtained was purified by flash column chromatography on silica gel column using 5% ethyl acetate in hexanes as eluents to afford Compound 20B as off white solid. Yield: 1.00 g (40.95%); LCMS m/z 580.39 [M+18]f.

To a solution Compound 18G (45.0 mg, 0.031 nmol) in dimethyl sulfoxide (1.0 mL) was added N,N-diisopropylethylamine (0.016 mL, 0.093 mmol) and Compound 20B (17.9 mg, 0.031 mmol). Reaction mixture was stirred at room temperature for 2 h. After completion, the reaction mixture was purified via preparatory HPLC (40-60% acetonitrile in water with 0.1% trifluoroacetic acid). Fractions containing the desired product were combined and lyophilized to dryness to afford Compound 1209 as an off white solid. Yield: 0.006 g (10.52%); LCMS m/z 1793.94 [M+1]⁻, 897.99 [M/2+1]⁺. ¹H NMR (400 MHz, DMSO-d6) δ 7.83 (t, J=5.6 Hz, 3H), 7.73 (t, J=5.2 Hz, 3H), 7.60 (d, J=9.2 Hz, 3H), 6.99 (s, 1H), 4.57-4.47 (m, 6H), 4.46 (d, J=4.4 Hz, 3H), 4.21 (d, J=8.4 Hz, 3H). 3.70-3.63 (m, 9H), 3.55-3.49 (m, 21H), 3.32-3.28 (m, 4H), 3.02 (t, J=5.6 Hz, 12H), 2.76 (t, J=5.6 Hz, 2H), 2.27 (t, J=6.4 Hz, 6H), 2.03 (t, J=7.2 Hz, 8H), 1.79 (s, 9H), 1.70-1.67 (m, 2H), 1.52-1.41 (m, 20H), 1.23 (bs, 14H).

### Synthesis of Compound 1209-C

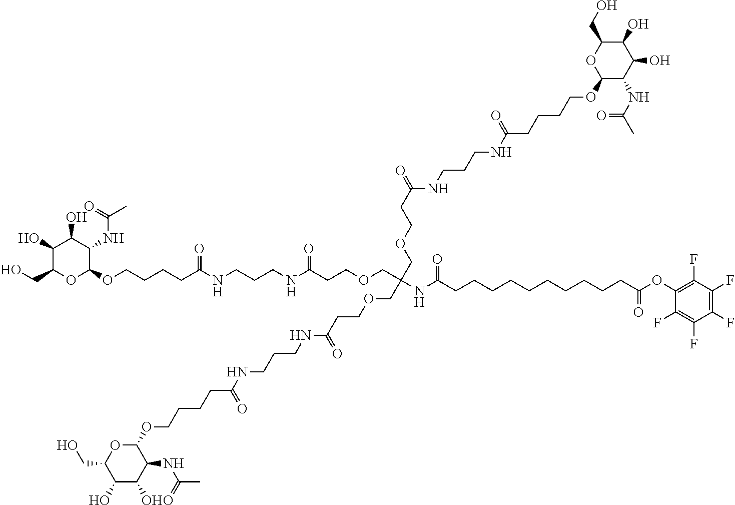 [SMILES: CC(=O)N[C@@H]1[C@@H](OCCCCC(=O)NCCCNC(=O)CCOCC(COCCC(=O)NCCCNC(=O)CCCCO[C@H]2O[C@@H](CO)[C@@H](O)[C@@H](O)[C@@H]2NC(C)=O)(COCCC(=O)NCCCNC(=O)CCCCO[C@H]2O[C@@H](CO)[C@@H](O)[C@@H](O)[C@@H]2NC(C)=O)NC(=O)CCCCCCCCCCC(=O)Oc2c(F)c(F)c(F)c(F)c2F)O[C@@H](CO)[C@@H](O)[C@H]1O]

Compound 1209-C was prepared analogously to compound 1209 using the synthetic route described for compound 1209, but using (3S,4S,5S,6S)-3-acetamido-6-(acetoxymethyl)tetrahydro-2H-pyran-2,4,5-triyl triacetate (6) as starting material in place of A-1 to give final product: LCMS, m/z 897.65 [M+2]⁻⁺.

### Synthesis of (3S,4S,5S,6S)-3-acetamido-6-(acetoxymethyl)tetrahydro-2H-pyran-2,4,5-triyl triacetate (6)

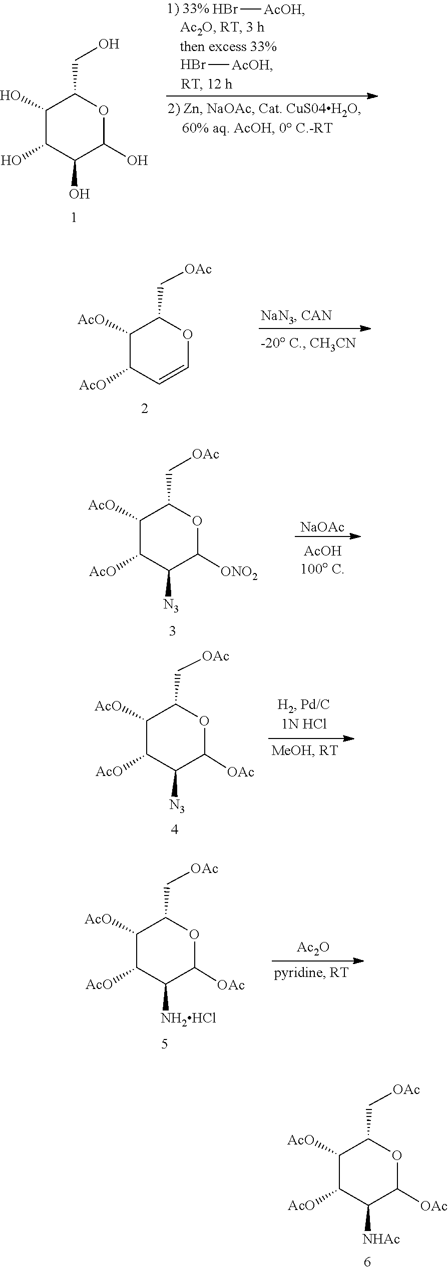

### Synthesis of (2S,3S,4S)-2-(acetoxymethyl)-3,4-dihydro-2H-pyran-3,4-diyl diacetate (2)

L-Galactose (5.9 g, 32.7 mmol) was suspended in a solution of acetic acid (15.0 mL) and acetic anhydride (21.7 mL, 7.0 eq, 229 mmol) and 33% HBr-acetic acid solution (6.0 g) was added. The reaction mixture was allowed to stir for 3 h at room temperature, after which 54.0 g more 33% HBr-acetic acid solution (total of 7.7 equiv HBr) was added and allowed to stir overnight. Sodium acetate was then added (22.0 g) to neutralize the excess HBr, and the reaction mixture was added to a suspension containing pulverized CuSO₄:5H₂O (1.92 g), zinc (6.3 g), water (60.0 mL), sodium acetate (54.8 g), and acetic acid (30.0 mL) and the resultant reaction mixture was stirred vigorously for 3 h. The solution was then filtered and the solid was washed with ethyl acetate (200 mL) and water (200 mL). The organic layer of the filtrate was then washed with saturated aqueous sodium bicarbonate solution (200 mL) and brine (100 mL), dried over sodium sulfate, filtered and concentrated under reduced pressure to provide crude compound. The crude product was purified by silica gel column chromatography (30% ethyl acetate-hexane) to give (2S,3S,4S)-2-(acetoxymethyl)-3,4-dihydro-2H-pyran-3,4-diyl diacetate (2) as colorless oil. Yield: 4.0 g, 45%, LCMS m/z 290.31 [M+18]⁺.

### Synthesis of (2S,3S,4S,5S)-2-(acetoxymethyl)-5-azido-6-(nitrooxy)tetrahydro-2H-pyran-3,4-diyl diacetate (3)

(2S,3S,4S)-2-(acetoxymethyl)-3,4-dihydro-2H-pyran-3,4-diyl diacetate (2, 4.0 g, 14.7 mmol) in dry acetonitrile (80 mL) was added to a mixture of ceric ammonium nitrate (32.2 g, 58.8 mmol) and sodium azide (1.91 g, 29.4 mmol) at −20° C. under nitrogen. After being stirred for 7 h at −20° C., the reaction mixture was diluted with cold diethyl ether and water, extracted with diethyl ether. The combined organic layer was washed with brine, dried over anhydrous sodium sulfate, filtered and concentrated under reduced pressure to yield crude (2S,3S,4S,5S)-2-(acetoxymethyl)-5-azido-6-(nitrooxy)tetrahydro-2H-pyran-3,4-diyl diacetate (3, 5.4 g) as light yellow syrup. The obtained crude mixture was used for the next reaction without further purification.

### Synthesis of (3S,4S,5S,6S)-6-(acetoxymethyl)-3-azidotetrahydro-2H-pyran-2,4,5-triyl triacetate (4)

To a solution of crude [(2S,3S,4S)-3,4-bis(acetyloxy)-5-azido-6-(nitrooxy)oxan-2-yl]methyl acetate (5.4 g) in acetic acid (50 mL) was added sodium acetate (3.53 g, 43.1 mmol) at room temperature under nitrogen atmosphere. After being stirred for 3 h at 100° C. the reaction mixture was cooled to 0° C., and diluted with ethyl acetate and saturated aqueous sodium bicarbonate, extracted with ethyl acetate. The combined organic layer was washed with brine, dried over anhydrous sodium sulfate, filtered and concentrated in vacuo. Purification by silica gel column chromatography (using 28-30% ethyl acetate/n-hexane) gave (3S,4S,5S,6S)-6-(acetoxymethyl)-3-azidotetrahydro-2H-pyran-2,4,5-triyl triacetate (4, 1.86 g, 34.64%) as a colorless viscous oil. Yield: 1.86 g, 34.64%, LCMS m/z 374.2 [M+1]⁺.

### Synthesis of (3S,4S,5S,6S)-6-(acetoxymethyl)-3-aminotetrahydro-2H-pyran-2,4,5-triyl triacetate hydrochloride (5)

To a stirred solution of (3S,4S,5S,6S)-6-(acetoxymethyl)-3-azidotetrahydro-2H-pyran-2,4,5-triyl triacetate (4, 1.8 g, 4.82 mmol) in methanol (40 mL) were added 10% Pd/C (1.8 g) and 1 N HCl aq. solution (5.79 mL, 5.79 mmol) at 0° C. After being stirred for 1 h at room temperature under hydrogen, the reaction mixture was filtered through a pad of Celite and concentrated in vacuo to give crude [(2S,3S,4S,5S)-3,4,6-tris(acetyloxy)-5-aminooxan-2-yl]methyl acetate hydrochloride (5) as an off-white solid. Yield: 1.7 g, 91.8%, LCMS m/z 348.1 [M+1]⁺.

### Synthesis of (3S,4S,5S,6S)-3-acetamido-6-(acetoxymethyl)tetrahydro-2H-pyran-2,4,5-triyl triacetate (6)

To a stirred solution of [(2S,3S,4S,5S)-3,4,6-tris(acetyloxy)-5-aminooxan-2-yl]methyl acetate hydrochloride (5, 1.7 g, 4.43 mmol) in pyridine (7.15 mL, 88.6 mmol) at 0° C. was added acetic anhydride (4.19 mL, 44.3 mmol) and stirred for 12 h at room temperature. The volatiles were then evaporated in reduced pressure. The crude material was purified by silica gel column chromatography (6.5% methanol in dichloromethane) to provide (3S,4S,5S,6S)-3-acetamido-6-(acetoxymethyl)tetrahydro-2H-pyran-2,4,5-triyl triacetate (6) as colorless syrup. Yield: 1.4 g, 81.17% LCMS m/z 448.1 [M+AcO]⁻.

### Synthesis of Compound 1226

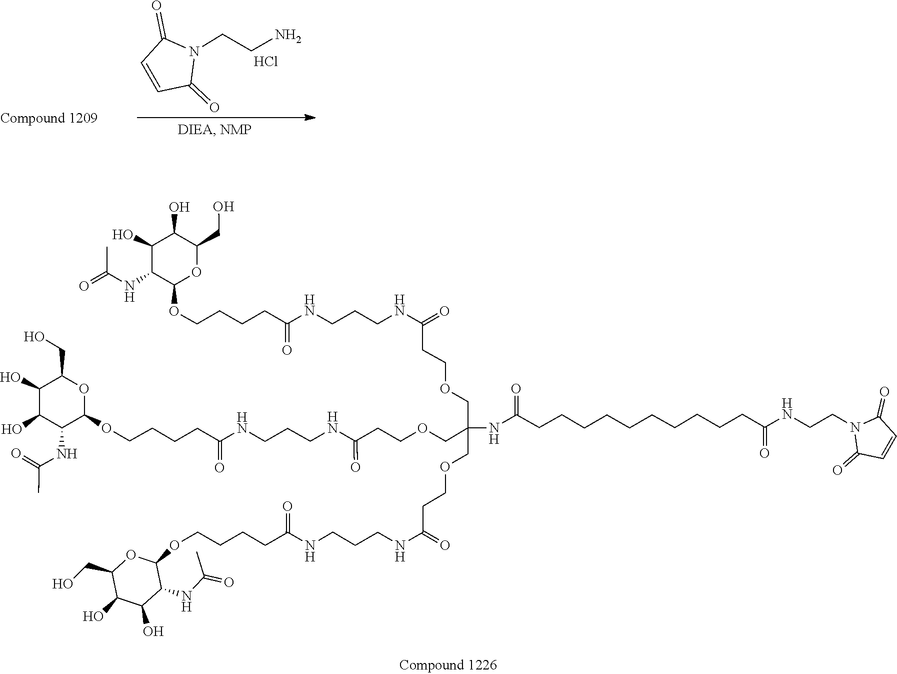 [SMILES: NCCN1C(=O)C=CC1=O ; Cl ; CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)NCCN2C(=O)C=CC2=O)O[C@H](CO)[C@H](O)[C@@H]1O]

To a mixture of Compound 1209 (1 eq, 17.2 mg, 0.00959 mmol) and 1-(2-aminoethyl) pyrrole-2,5-dione;hydrochloride (1.1 eq. 1.86 mg, 0.00105 mmol) in NMP (0.5 mL) was added DIEA (3 eq. 5 μL, 0.0288 mmol). The mixture was stirred at rt for 10 minutes and acetic acid (4 μL) was added. The mixture was purified by prep. HPLC (10-40% MeCN/water with 0.1% TFA) to give compound 1226 as a white solid (10.1 mg, yield 64%). (purity: 99%). LCMS m/z 1751.0 [M+H]+.

### Synthesis of Compound Y

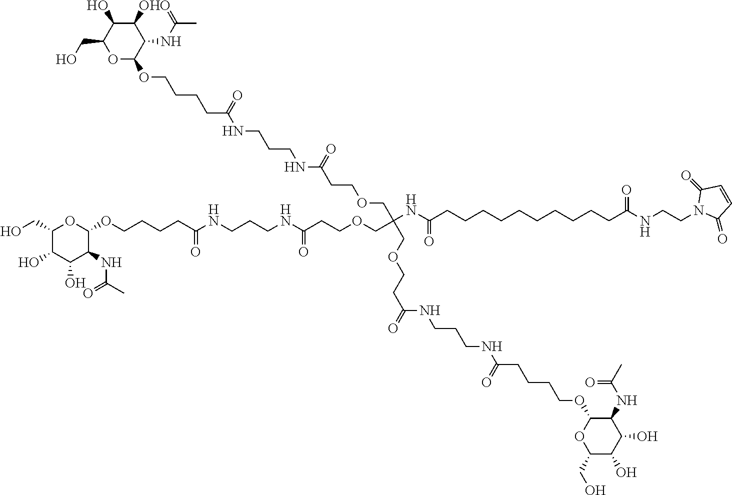 [SMILES: CC(=O)N[C@@H]1[C@@H](OCCCCC(=O)NCCCNC(=O)CCOCC(COCCC(=O)NCCCNC(=O)CCCCO[C@H]2O[C@@H](CO)[C@@H](O)[C@@H](O)[C@@H]2NC(C)=O)(COCCC(=O)NCCCNC(=O)CCCCO[C@H]2O[C@@H](CO)[C@@H](O)[C@@H](O)[C@@H]2NC(C)=O)NC(=O)CCCCCCCCCCC(=O)NCCN2C(=O)C=CC2=O)O[C@@H](CO)[C@@H](O)[C@H]1O]

Compound Y was prepared analogously to compound 1226 using synthetic routes described for compounds 1209 and 1226, but using (3S,4S,5S,6S)-3-acetamido-6-(acetoxymethyl)tetrahydro-2H-pyran-2,4,5-triyl triacetate (6) as starting material in place of A-1 to give final product: LCMS m/z 1750.9 [M+H]+.

### Synthesis of XB47 (Intermediate for Compound 1254 and Compound 2346)

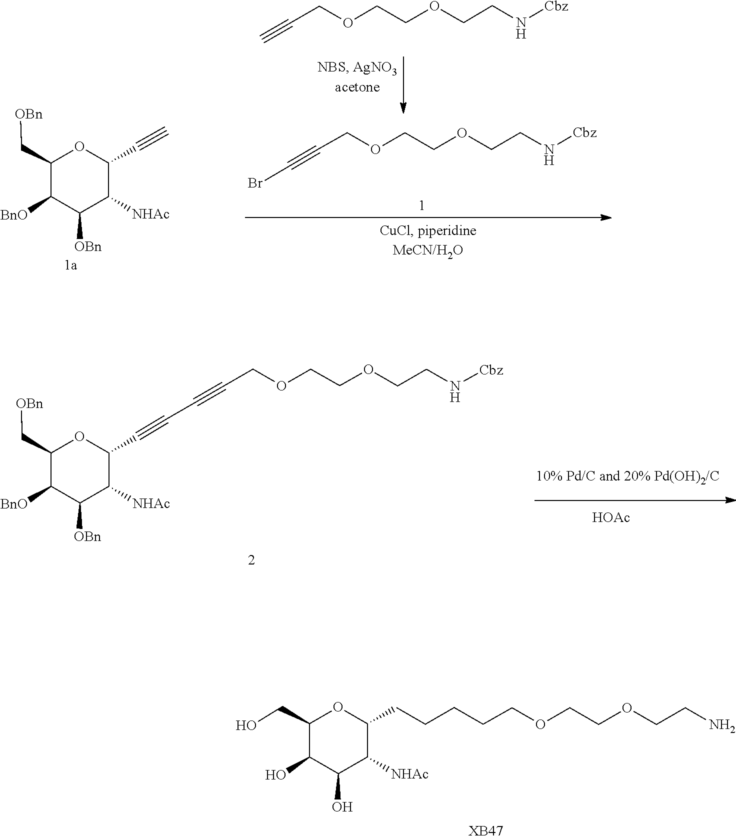 [SMILES: C#CCOCCOCCNC(=O)OCc1ccccc1 ; C#C[C@H]1O[C@H](COCc2ccccc2)[C@H](OCc2ccccc2)[C@H](OCc2ccccc2)[C@H]1NC(C)=O ; O=C(NCCOCCOCC#CBr)OCc1ccccc1 ; CC(=O)N[C@@H]1[C@@H](OCc2ccccc2)[C@@H](OCc2ccccc2)[C@@H](COCc2ccccc2)O[C@@H]1C#CC#CCOCCOCCNC(=O)OCc1ccccc1 ; CC(=O)N[C@@H]1[C@@H](O)[C@@H](O)[C@@H](CO)O[C@@H]1CCCCCOCCOCCN ; "HOAc"]

To a mixture of benzyl N-[2-(2-prop-2-ynoxyethoxy)ethyl]carbamate (1.00 eq, 816 mg, 2.94 mmol) in acetone (29 mL) were added NBS (1.34 eq, 704 mg, 3.95 mmol) and silver nitrate (0.144 eq, 72.0 mg, 0.424 mmol). The mixture was stirred at room temperature for 1 h and concentrated. The residue was diluted with EtOAC and washed with water (1×). The aqueous layer was extracted with EtOAc (2×). The combined organic layers were washed with brine, dried, concentrated, purified by column (0-50% EtOAc/hexane) to give 1 as clear oil (930 mg, yield: 89%). LCMS m/z 378.0 [M+Na]+.

To a mixture of N-[(2R,3S,4R,5R,6R)-4,5-dibenzyloxy-6-(benzyloxymethyl)-2-ethynyl-tetrahydropyran-3-yl]acetamide (prepared by literature routes described in Rouzier, et al, Synthesis (2019), 51(12), 2484-2488,) (ISP2-718, 1.00 eq, 201 mg, 0.403 mmol) 1 in MeCN (7.6 mL) and water (4.4 mL) was added benzyl N-[2-[2-(3-bromoprop-2-ynoxy)ethoxy]ethyl]carbamate (1, 1.20 eq, 172 mg, 0.484 mmol). The mixture was cooled to 0° C. and piperidine (5.00 eq, 0.20 mL, 2.02 mmol) was added. The mixture was purged with N₂ and CuCl (0.240 eq, 9.6 mg, 0.0967 mmol) was added. The mixture was slowly wanned to room temperature and stirred at room temperature overnight. The mixture was diluted with EtOAc, washed with 10% citric acid (1×) and brine (1×), dried, concentrated, purified by column (0-80% EtOAC/hexane) to give 2 as a white solid (232.2 mg, yield: 74%). LCMS m/z 775.2 [M+H]+.

To a mixture of benzyl N-[2-[2-[5-[(2R,3S,4R,5R,6R)-3-acetamido-4,5-dibenzyloxy-6-(benzyloxymethyl)tetrahydropyran-2-yl]penta-2,4-diynoxy]ethoxy]ethyl]carbamate (2, 1.00 eq, 232 mg, 0.300 mmol) in HOAc (6 mL) were added 10% Pd/C (220 mg) and 20% Pd(OH)₂/C (230 mg). The mixture was stirred at room temperature under hydrogen for 2.5 h, filtered, concentrated, and purified by prep. HPLC (2-40% MeCN/20 mM NH₄OH aqueous solution) to give XB47 as a white solid (83.6 mg, yield: 74%). LCMS m/z 379.3 [M+H]+.

### Synthesis of Compound 1254

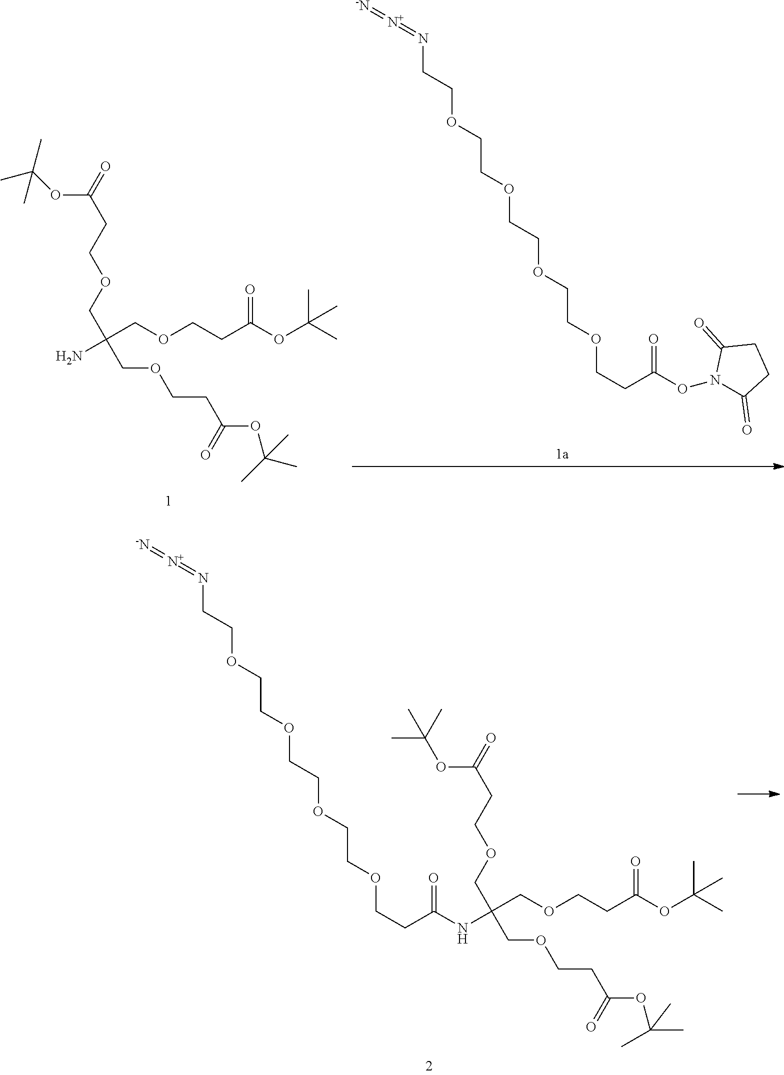

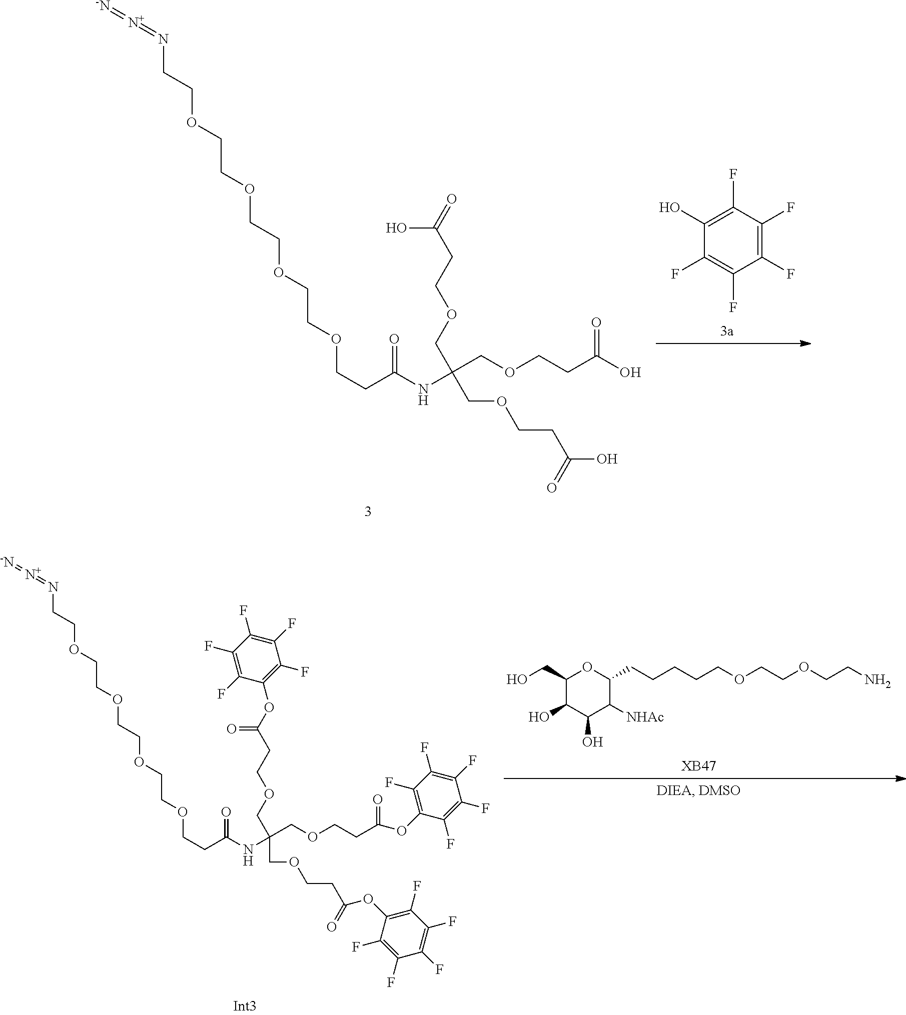

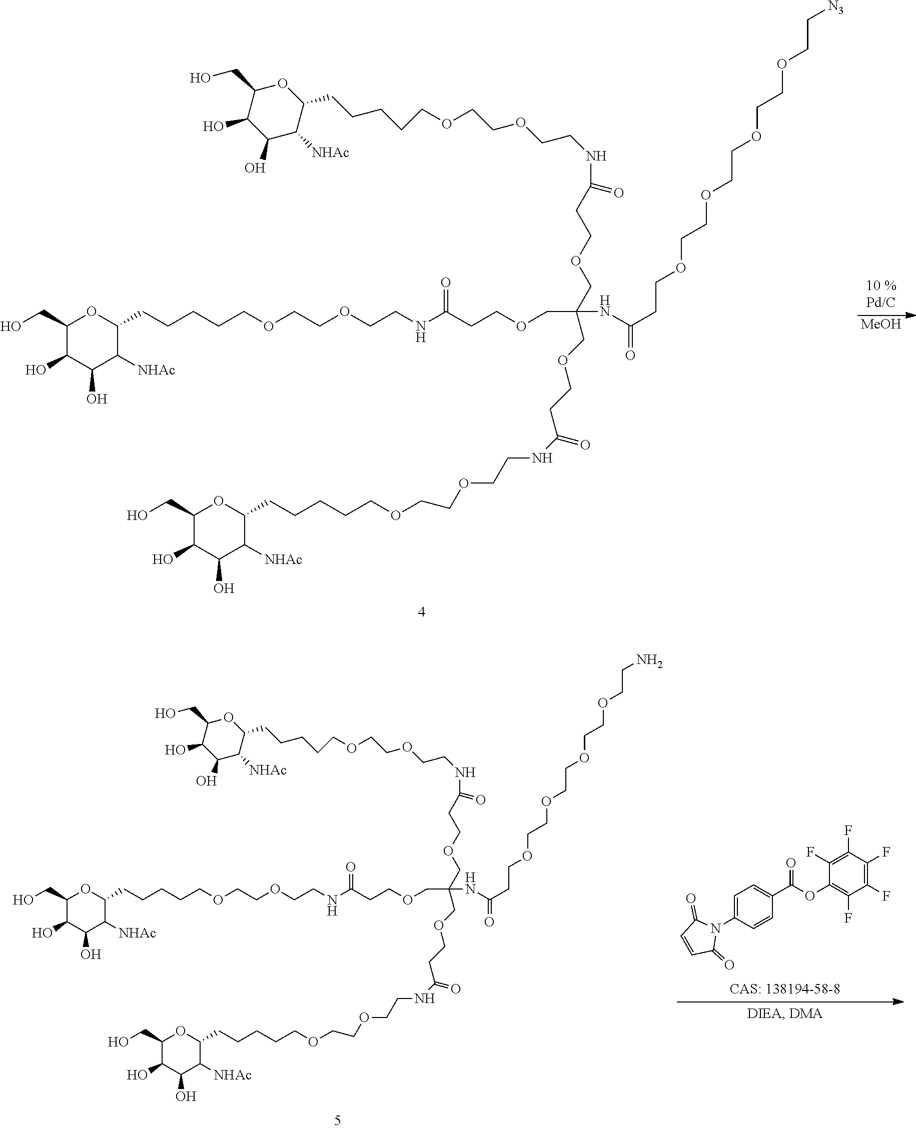

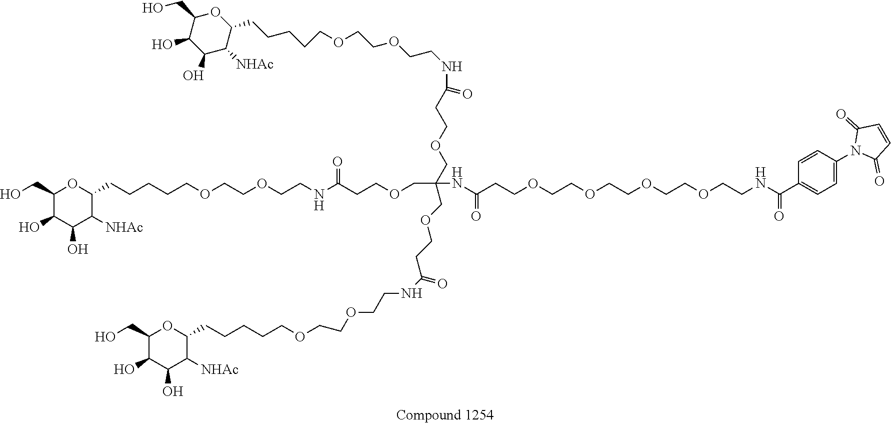 [SMILES: CC(=O)NC1[C@@H](O)[C@@H](O)[C@@H](CO)O[C@@H]1CCCCCOCCOCCNC(=O)CCOCC(COCCC(=O)NCCOCCOCCCCC[C@H]1O[C@H](CO)[C@H](O)[C@H](O)C1NC(C)=O)(COCCC(=O)NCCOCCOCCCCC[C@H]1O[C@H](CO)[C@H](O)[C@H](O)[C@H]1NC(C)=O)NC(=O)CCOCCOCCOCCOCCNC(=O)c1ccc(N2C(=O)C=CC2=O)cc1]

### Synthesis of tert-butyl 1-azido-17,17-bis((3-(tert-butoxy)-3-oxopropoxy)methyl)-15-oxo-3,6,9,12,19-pentaoxa-16-azadocosan-22-oate (2)

A solution of di-tert-butyl 3,3′-((2-amino-2-((3-(tert-butoxy)-3-oxopropoxy)methyl)propane-1,3-diyl)bis(oxy))dipropionate (1) (452 mg, 0.894 mmol, 1.00 eq.) and 2,5-dioxopyrrolidin-1-yl 1-azido-3,6,9,12-tetraoxapentadecan-15-oate (1a) (356 mg, 0.916 mmol, 1.02 eq.) in 1 mL acetonitrile was stirred under nitrogen atmosphere and heated at 45° C. for 2 days until minimal 1 remained. The reaction mixture was diluted with dichloromethane and evaporated onto silica, then purified by flash column chromatography column, eluting with 0-100% ethyl acetate/dichloromethane. Fractions containing product were concentrated and dried further under high vacuum at ambient temperature to afford 2 as a clear thick syrup. Yield: 559 mg (78%); LCMS m/z 779.2 [M+H].

### Synthesis of 1-azido-17,17-bis((2-carboxyethoxy)methyl)-15-oxo-3,6,9,12,19-pentaoxa-16-azadocosan-22-oic acid (3)

To tert-butyl 1-azido-17,17-bis((3-(tert-butoxy)-3-oxopropoxy)methyl)-15-oxo-3,6,9,12,19-pentaoxa-16-azadocosan-22-oate (2) (509 mg, 0.654 mmol) was added 10 mL of a pre-mixed solution of 30% trifluoroacetic acid in dichloromethane. The mixture stirred at ambient temperature for 3.5 hrs., until consumption of Compound 2. The solvent was evaporated to residue under a stream of nitrogen, dried further under high vacuum at ambient temperature, then dissolved in 20% water/acetonitrile and lyophilized to dryness to afford 3 as a thick dark yellow color syrup. Yield: 424 mg (99%); LCMS m/z 611.3 [M+H], 609.4 [M−1]−.

A solution of 1-azido-17,17-bis((2-carboxyethoxy)methyl)-15-oxo-3,6,9,12,19-pentaoxa-16-azadocosan-22-oic acid (3) (81.3 mg, 0.133 mmol, 1.0 eq.) and 2,3,4,5,6-pentafluorophenol (3a) (91.8 mg, 0.499 mmol, 3.7 eq.) in 1.5 mL of dichloromethane was added N,N′-diisopropylcarbodiimide (90 mL, 0.581 mmol, 4.4 eq.). The mixture stirred at ambient temperature for 1 hr, until consumption of Compound 3. The reaction mixture was diluted with dichloromethane, evaporated onto silica, then purified by flash column chromatography column, eluting with 0-100% ethyl acetate/hexanes. Fractions containing product were concentrated and dried further under high vacuum at ambient temperature to afford Int3 as a clear thick syrup. Yield: 104 mg (67%); LCMS m/z 1109.1 [M+H].

### Synthesis of (4)

To a mixture of perfluorophenyl 1-azido-15-oxo-17,17-bis((3-oxo-3-(perfluorophenoxy)propoxy)methyl)-3,6,9,12,19-pentaoxa-16-azadocosan-22-oate (Int3, 1.00 eq, 18.9 mg, 0.017 mmol) and N-[(2R,3R,4R,5R,6R)-2-[5-[2-(2-aminoethoxy)ethoxy]pentyl]-4,5-dihydroxy-6-(hydroxymethyl)tetrahydropyran-3-yl]acetamide (XB47, 3.50 eq, 22.6 mg, 0.0597 mmol) in DMSO (0.7 mL) was added DIPEA (10.0 eq, 0.030 mL, 0.170 mmol). The mixture was stirred at room temperature for 2 h and was purified by prep. HPLC (2-45% MeCN/water with 0.1% TFA) to give 4 as a white solid. Yield: 21 mg (73%): LCMS: 1691.4 [M+H]+.

### Synthesis of (5)

To a mixture of 4 (1.00 eq, 21.0 mg, 0.0124 mmol) in MeOH (3 mL) was added 10% Pd/C (8 mg). The mixture was stirred at room temperature under hydrogen for 1 h, filtered, and concentrated to give 5 as a white solid. Yield: 19.6 mg (95%): LCMS: 1665.8 [M+H]+.

### Synthesis of Compound 1254

To a mixture of (2,3,4,5,6-pentafluorophenyl) 4-(2,5-dioxopyrrol-1-yl)benzoate (CAS: 138194-58-8, 1.10 eq, 2.5 mg, 0.0066 mmol) in DMA (0.1 mL) at 0° C. was added a solution of 5 (1.00 eq, 10.0 mg, 0.00600 mmol) in DMA (0.1 mL) dropwise, followed by addition of DIPEA (5.00 eq, 0.0052 mL, 0.0300 mmol) The mixture was stirred at room temperature for 20 minutes. The mixture was purified by prep. HPLC (2-50% MeCN/water with 0.1% TFA) to give Compound 1254 as a white solid. Yield: 6.5 mg (58%); LCMS: 1865.6 [M+H]+, 1863.6 [M−H]−.

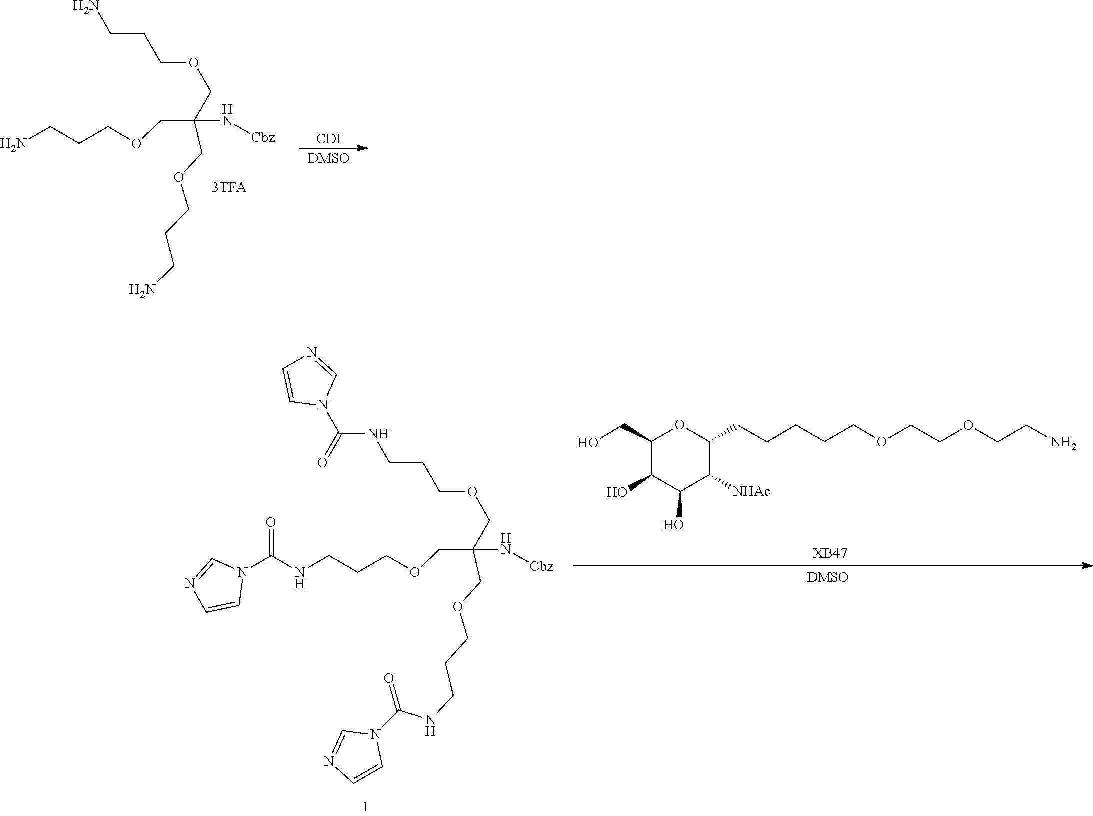

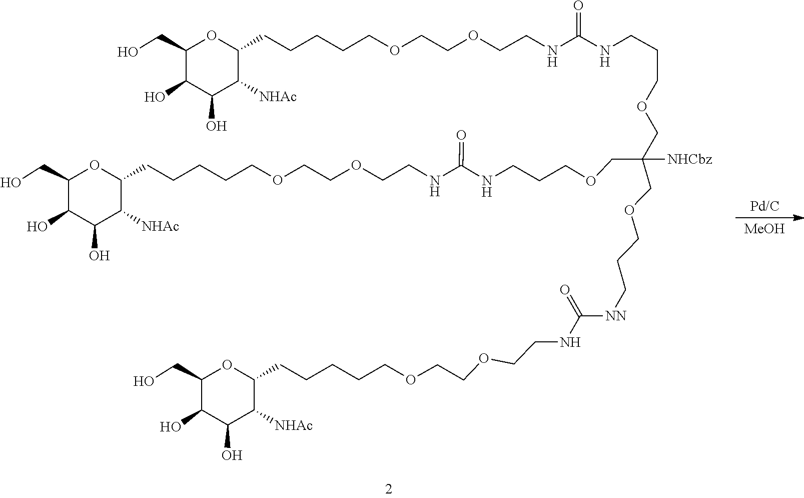

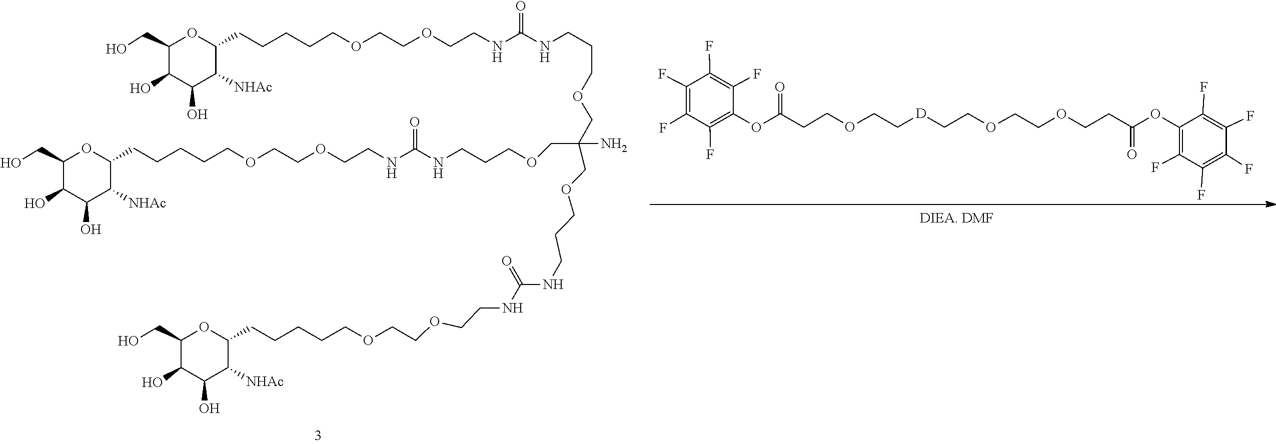

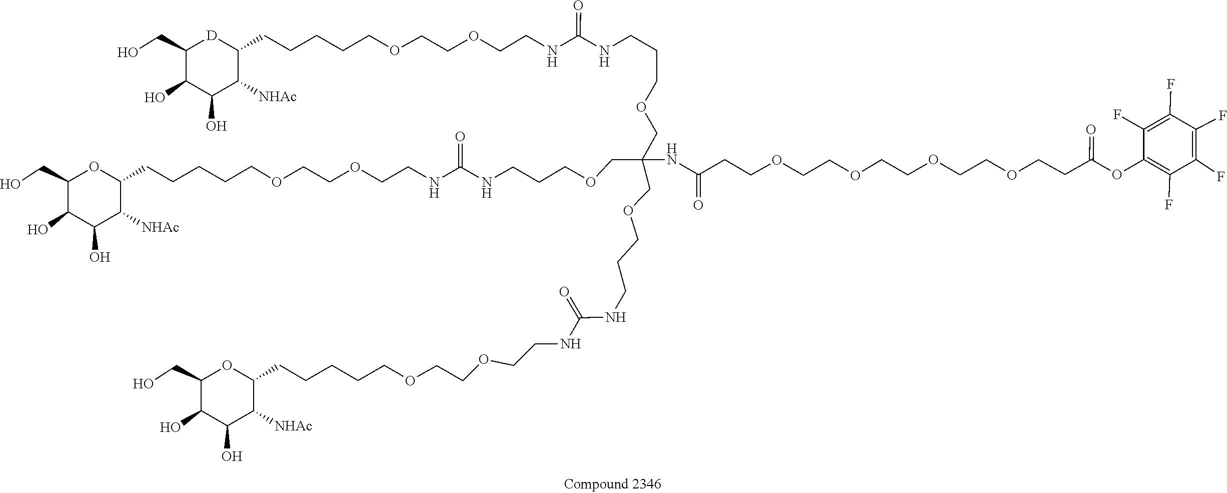

### Synthesis of benzyl (8-((3-(1H-imidazole-1-carboxamido)propoxy)methyl)-1,15-di(1H-imidazol-1-yl)-1,15-dioxo-6,10-dioxa-2,14-diazapentadecan-8-yl)carbamate (1)

To a solution of carbonyldiimidazole (7.00 eq, 142 mg, 0.875 mmol) in DMSO (0.4 mL) was added a mixture of benzyl (1,3-bis(3-aminopropoxy)-2-((3-aminopropoxy)methyl)propan-2-yl)carbamate;2,2,2-trifluoroacetic acid, which can be prepared by literature routes described in Sun, Chengzao; et al Bioorganic & Medicinal Chemistry Letters (2002), 12(16), 2213-2215, (1.00 eq, 96.1 mg, 0.125 mmol) in DMSO (1.3 mL). The mixture was stirred at rt for 2 h, purified by prep. HPLC (5-90% MeCN/water) and re-purified by column (0-10% MeOH/DCM) to give 1 as clear syrup. Yield: 55 mg (62%). LCMS m/z 573.1 [M-Cbz]

Synthesis of benzyl (1,39-bis((2R,3R,4R,5R,6R)-3-acetamido-4,5-dihydroxy-6-(hydroxymethyl)tetrahydro-2H-pyran-2-yl)-20-(19-((2R,3R,4R,5R,6R)-3-acetamido-4,5-dihydroxy-6-(hydroxymethyl)tetrahydro-2H-pyran-2-yl)-7-oxo-2,11,14-trioxa-6,8-diazanonadecyl)-13,27-dioxo-6,9,18,22,31,34-hexaoxa-12,14,26,28-tetraazanonatriacontan-20-yl)carbamate (2):

To a mixture of benzyl (8-((3-(1H-imidazole-1-carboxamido)propoxy)methyl)-1,15-di(1H-imidazol-1-yl)-1,15-dioxo-6,10-dioxa-2,14-diazapentadecan-8-yl)carbamate (1, 1.00 eq, 15.1 mg, 0.0213 mmol) in DMSO (0.25 mL) was added a solution of N-((2R,3R,4R,5R,6R)-2-(5-(2-(2-aminoethoxy)ethoxy)pentyl)-4,5-dihydroxy-6-(hydroxymethyl)tetrahydro-2H-pyran-3-yl)acetamide (ITX0025438, 3.00 eq, 24.2 mg, 0.0639 mmol). The mixture was stirred at 50° C. overnight and purified by prep. HPLC (5-12-30% MeCN/water with 0.1% TFA) to give 2 as a white solid. Yield: 32 mg (92%); LCMS 1641 [M+H].

### Synthesis of N-[(2R,3R,4R,5R,6R)-2-[5-[2-[2-[3-[3-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxy]-2-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxymethyl]-2-amino-propoxy]propylcarbamoylamino]ethoxy]ethoxy]pentyl]-4,5-dihydroxy-6-(hydroxymethyl)tetrahydropyran-3-yl]acetamide (3)

To a mixture of benzyl (1,39-bis((2R,3R,4R,5R,6R)-3-acetamido-4,5-dihydroxy-6-(hydroxymethyl)tetrahydro-2H-pyran-2-yl)-20-(19-((2R,3R,4R,5R,6R)-3-acetamido-4,5-dihydroxy-6-(hydroxymethyl)tetrahydro-2H-pyran-2-yl)-7-oxo-2,11,14-trioxa-6,8-diazanonadecyl)-13,27-dioxo-6,9,18,22,31,34-hexaoxa-12,14,26,28-tetraazanonatriacontan-20-yl)carbamate (2, 1.00 eq, 28.4 mg, 0.0173 mmol) in MeOH (4 mL) was added 10% Pd/C (10 mg). The mixture was stirred at rt under hydrogen for 1 h, filtered and concentrated to give 3 as a white solid. Yield: 25.2 mg (97%); LCMS 1505.4 [M+H].

### Synthesis of perfluorophenyl 37-((2R,3R,4R,5R,6R)-3-acetamido-4,5-dihydroxy-6-(hydroxymethyl)tetrahydro-2H-pyran-2-yl)-18,18-bis(19-((2R,3R,4R,5R,6R)-3-acetamido-4,5-dihydroxy-6-(hydroxymethyl)tetrahydro-2H-pyran-2-yl)-7-oxo-2,11,14-trioxa-6,8-diazanonadecyl)-16,25-dioxo-4,7,10,13,20,29,32-heptaoxa-17,24,26-triazaheptatriacontanoate (Compound 2346)

To a mixture of N-[(2R,3R,4R,5R,6R)-2-[5-[2-[2-[3-[3-[3-[2-[2-[5-[(2R,3R,4R,5R,6R)- 3-acetamido-4,5-dihydroxy-6-(hydroxymethyl)tetrahydropyran-2-yl]pentoxy]ethoxy]ethylcarbamoylamino]propoxy]-2-[3-[2-[2-[5-[(2R,3R,4R,5R,6R)-3-acetamido-4,5-dihydroxy-6-(hydroxymethyl)tetrahydropyran-2-yl]pentoxy]ethoxy]ethylcarbamoylamino]propoxymethyl]-2-amino-propoxy]propylcarbamoylamino]ethoxy]ethoxy]pentyl]-4,5-dihydroxy-6-(hydroxymethyl)tetrahydropyran-3-yl]acetamide (3, 1.00 eq, 9.6 mg, 0.0064 mmol) in DMF (0.1 mL) was added a solution of Bis-PEG4-PFP ester (1.5 eq, 6.0 mg, 0.0096 mmol) in DMF (0.15 mL) and Diisopropylethylamine (DIPEA) (2.0 eq, 0.0022 mL, 0.0128 mmol). The mixture was stirred at rt overnight and was purified by prep. HPLC (5-50% MeCN/water with 0.1% TFA) to give Compound 2346 as a white solid. Yield: 5.24 mg (42%); LCMS 1948.6 [M+H].

### Example 1. Depletion of Extracellular Target Molecules Using an Exemplary Target Binding Conjugate

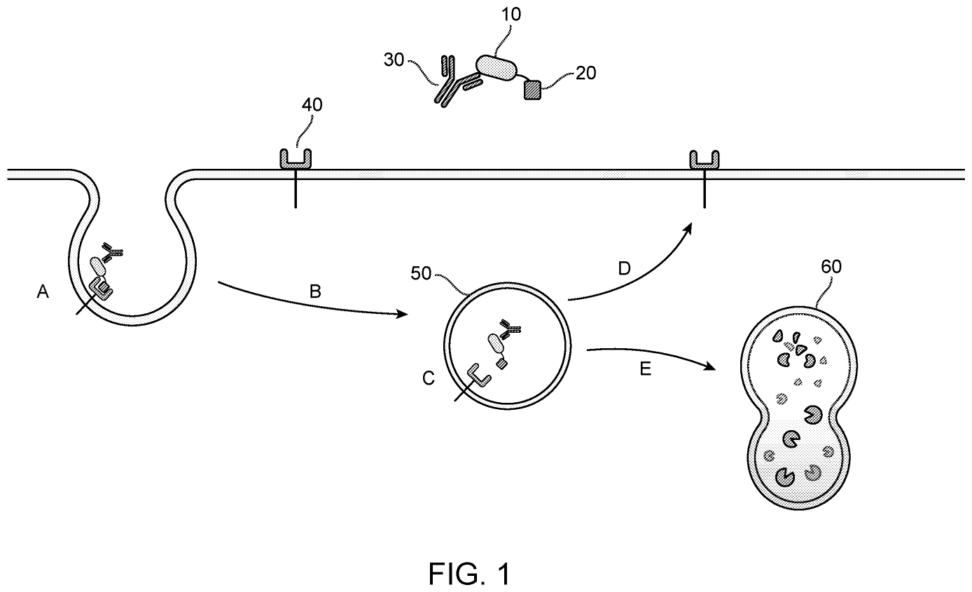

FIG. 1 shows a hypothesized mechanism for uptake and degradation of a target by bifunctional molecules described herein. The bifunctional molecule shown consists of polypeptide 10 and moiety 20 that binds to a lysosomal targeting molecule. Polypeptide 10 and moiety 20 correspond to B and X respectively in formula (I). Polypeptide 10 of the bifunctional molecule binds to a target molecule 30. Moiety 20 binds lysosomal targeting molecule 40 (e.g. M6PR, ASGPR). In step A, a ternary complex (a bifunctional molecule, a target, and a lysosomal targeting molecule) is internalized into a cell. e.g. via clathrin-mediated endocytosis. In step B, endosome 50 is formed. The bifunctional molecule then dissociates from the lysosomal targeting molecule in step C due to decreasing pH or a decreasing concentration of Ca²⁺. The lysosomal targeting molecule returns to the cell surface via step D. and the bifunctional molecule and target progress via step E to lysosome 60 and are degraded.

### Example 2. Display of Antigen Domains

FIGS. 2A-D show various displays of antigen domains capable of binding to antibody targets (e.g. autoantibodies). The domains were designed and expressed as genetic fusions containing N/C-terminal tags including human Fc (FIGS. 2A & 2B), and human serum albumin with 6×-poly-histidine (SEQ ID NO: 2) and without a 6×-poly-histidine (SEQ ID NO: 1) (FIGS. 2C & 2D).

Referring to FIG. 2A. Fc fusions were generated as homo-dimeric fusions with globular targets containing a Gly₄Ser linker, expressed in either Expi293 of ExpiCHO cells containing a C-terminal human IgG1-Fc domain including the hinge domain. Before purification, the conditioned material was filtered with a 0.22 uM clarification filter and then loaded on a 5 mL Hi Trap mAb Select sure column pre-equilibrated with 1×PBS (pH 7.4). After loading, the column was washed with 1×PBS until A280 achieved baseline, then the protein was eluted with 50 mM Acetate pH 3.5, followed by neutralization with addition of 1M Tris pH 8.0. In the second step, the Protein A pool was loaded on either a superdex 75 or superdex 200 (10/300 or 26/60) equilibrated in 1×PBS (pH 7.5). The eluted monomeric protein was collected and pooled based on gel and A280.

Referring to FIG. 2B. Fc fusions were generated as monovalent Fc-fusions, with globular targets designed as C-terminal scFc fusions containing a Gly₄Ser linker of sufficient length between the FEc domains and expressed in E. coli (BL21 DE3) and expressed into inclusion bodies. After cell rupture and clarification, the inclusion bodies were extensively washed and then solubilized in 50 mM Tris pH 8.5, 8M Urea, 8 mM DTT. Solubilized inclusion bodies were then refolded in 50 mM Tris pH 8.5, 0M-4M Urea, 3 mM Cysteine, 1 mM Cystamine, 160 mM L-Arginine and left shaking over night at room temperature. The refolded Fc-fusions were then diluted 1 fold with Milli Q water, and filtered with a 0.22 uM clarification filter and then loaded on a 5 mL Hi Trap mAb Select sure column pre-equilibrated with 1×PBS (pH 7.4). After loading, the column was washed with 1×PBS (pH 7.4) until A280 achieved baseline, then the protein was eluted with 50 mM Acetate pH 3.5, followed by neutralization with addition of 1M Tris pH 8.0. In the second step, the Protein A pool was loaded on either a superdex 75 or superdex 200 (10/300 or 26/60) equilibrated in 1×PBS (pH 7.4). The eluted monomeric protein was collected and pooled based on gel and A280.

Referring to FIG. 2C, antigens were generated with a C-terminal HSA as a genetic fusion partner, and were expressed in either Expi293 of ExpiCHO cells. Protein was purified from conditioned media on an AKTA Go using a combination of affinity purification and gel filtration. For purification, the conditioned material was filtered affinity purified using Thasmo HSA capture select resin, washed with 1×PBS, and eluted with 100 mM Glycine pH 3.2. In the second stephashe HSA pool was loaded on either a superdex 75 or superdex 200 (10/300 or 26/60) equilibrated in 1×PBS (pH 7.5). The eluted monomeric protein was collected and pooled based on gel and A280.

Referring to FIG. 2D, antigens were generated with a C-terminal HSA as a genetic fusion partner containing a 6×-poly-histidine, and were expressed in either Expi293 of ExpiCHO cells. Protein was purified from conditioned media on an AKTA Go using a combination of affinity purification and gel filtration. For Prior purification, the conditioned material was filtered and conditioned with 1 mM MgCl2, plus 30 mM Imidazole and pH adjusted to pH 7.5-8.0. In the first step, the protein was loaded onto a 5 mL HiTrap IMAC column (Ni2+ charged). The bound protein was washed in 1×PBS+30 mM Imidizole (pH 8.0) until A280 baseline and eluted with 1×PBS+300 mM Inidizole (pH 8.0). In the second stephashe HSA pool was loaded on either a superdex 75 or superdex 200 (10/300 or 26/60) equilibrated in 1×PBS (pH 7.5). The eluted monomeric protein was collected and pooled based on gel and A280.

Sequences of exemplary antigen protein, carrier polypeptides, linkers, and fusion proteins generated by methods described herein, are shown below in Table 26:

TABLE 26

| | |
|---|---|
| Sequence | SEQ ID NO |

Carrier proteins

| | | |
|---|---|---|
| Human | DAHKSEVAHRFKDLGEENFKALVLIAFAQYLQQCPFEDHVKLVN | 1 |
| Serum | EVTEFAKTCVADESAENCDKSLHTLFGDKLCTVATLRETYGEMA | |
| Albumin | DCCAKQEPERNECFLQHKDDNPNLPRLVRPEVDVMCTAFHDNEE | |
| (HSA) | TFLKKYLYEIARRHPYFYAPELLFFAKRYKAAFTECCQAADKAAC | |
| | LLPKLDELRDEGKASSAKQRLKCASLQKFGERAFKAWAVARLSQ | |
| | RFPKAEFAEVSKLVTDLTKVHTECCHGDLLECADDRADLAKYICE | |
| | NQDSISSKLKECCEKPLLEKSHCIAEVENDEMPADLPSLAADFVES | |
| | KDVCKNYAEAKDVFLGMFLYEYARRHPDYSVVLLLRLAKTYETT | |
| | LEKCCAAADPHECYAKVFDEFKPLVEEPQNLIKQNCELFEQLGEY | |
| | KFQNALLVRYTKKVPQVSTPTLVEVSRNLGKVGSKCCKHPEAKR | |
| | MPCAEDYLSVVLNQLCVLHEKTPVSDRVTKCCTESLVNRRPCFSA | |
| | LEVDETYVPKEFNAETFTFHADICTLSEKERQIKKQTALVELVKHK | |
| | PKATKEQLKAVMDDFAAFVEKCCKADDKETCFAEEGKKLVAAS | |
| | QAALGL | |
| Human | DAHKSEVAHRFKDLGEENFKALVLIAFAQYLQQCPFEDHVKLVN | 2 |
| Serum | EVTEFAKTCVADESAENCDKSLHTLFGDKLCTVATLRETYGEMA | |
| Albumin | DCCAKQEPERNECFLQHKDDNPNLPRLVRPEVDVMCTAFHDNEE | |
| with His-tag | TFLKKYLYEIARRHPYFYAPELLFFAKRYKAAFTECCQAADKAAC | |
| | LLPKLDELRDEGKASSAKQRLKCASLQKFGERAFKAWAVARLSQ | |
| | RFPKAEFAEVSKLVTDLTKVHTECCHGDLLECADDRADLAKYICE | |
| | NQDSISSKLKECCEKPLLEKSHCIAEVENDEMPADLPSLAADFVES | |
| | KDVCKNYAEAKDVFLGMFLYEYARRHPDYSVVLLLRLAKTYETT | |
| | LEKCCAAADPHECYAKVFDEFKPLVEEPQNLIKQNCELFEQLGEY | |
| | KFQNALLVRYTKKVPQVSTPTLVEVSRNLGKVGSKCCKHPEAKR | |
| | MPCAEDYLSVVLNQLCVLHEKTPVSDRVTKCCTESLVNRRPCFSA | |
| | LEVDETYVPKEFNAETFTFHADICTLSEKERQIKKQTALVELVKHK | |
| | PKATKEQLKAVMDDFAAFVEKCCKADDKETCFAEEGKKLVAAS | |
| | QAALGLGGHHHHHH | |
| Human Fc | DKTHTCPPCPAPEAAGGPSVFLFPPKPKDTLMISRTPEVTCVVVDV | 3 |
| | SHEDPEVKFNWYVDGVEVHNAKTKPREEQYNSTYRVVSVLTVL | |
| | HQDWLNGKEYKCKVSNKALPAPIEKTISKAKGQPREPQVYTLPPS | |
| | REEMTKNQVSLTCLVKGFYPSDIAVEWESNGQPENNYKTTPPVLD | |
| | SDGSFFLYSKLTVDKSRWQQGNVFSCSVMHEALHNHYTQKSLSL | |
| | SPGK | |
| Human scFc | DKTHTCPPCPAPELLGGPSVFLFPPKPKDTLMISRTPEVTCVVVDV | 4 |
| | SHEDPEVKFNWYVDGVEVHNAKTKPCEEQYGSTYRCVSVLTVLH | |
| | QDWLNGKEYKCKVSNKALPAPIEKTISKAKGQPREPQVYTLPPSR | |
| | EEMTKNQVSLTCLVKGFYPSDIAVEWESNGQPENNYKTTPPVLDS | |
| | DGSFFLYSKLTVDKSRWQQGNVFSCSVMHEALHNHYTQKSLSLS | |
| | PGKGGGGSGGGGSGGGGSGGGGSGGGGSGGGGSDKTHTCPPCP | |
| | APELLGGPSVFLFPPKPKDTLMISRTPEVTCVVVDVSHEDPEVKFN | |
| | WYVDGVEVHNAKTKPCEEQYGSTYRCVSVLTVLHQDWLNGKEY | |
| | KCKVSNKALPAPIEKTISKAKGQPREPQVYTLPPSREEMTKNQVSL | |
| | TCLVKGFYPSDIAVEWESNGQPENNYKTTPPVLDSDGSFFLYSKL | |
| | TVDKSRWQQGNVFSCSVMHEALHNHYTQKSLSLSPGK | |

Linkers

| | | |
|---|---|---|
| G4S | GGGGS | 5 |
| 2XG4S | GGGGSGGGGS | 6 |
| 3XG4S | GGGGSGGGGSGGGGS | 7 |
| 4XG4S | GGGGSGGGGSGGGGSGGGGS | 8 |
| 5XG4S | GGGGSGGGGSGGGGSGGGGSGGGGS | 9 |

Antigen proteins

| | | |
|---|---|---|
| MuSK-D1 | PVITTPLETVDALVEEVATFMCAVESYPQPEISWTRNKILIKLFDTR | 10 |
| (MuSK IG- | YSIRENGQLLTILSVEDSDDGIYCCTANNGVGGAVESCGALQVKM | |
| like domain | K | |
| 1-P28-K120) | | |
| Musk-D1D2 | EKLPKAPVITTPLETVDALVEEVATFMCAVESYPQPEISWTRNKILI | 11 |
| (MuSK Ig- | KLFDTRYSIRENGQLLTILSVEDSDDGIYCCTANNGVGGAVESCG | |
| like domains | ALQVKMKPKITRPPINVKIIEGLKAVLPCTTMGNPKPSVSWIKGDS | |
| 1/2-E22- | PLRENSRIAVLESGSLRIHNVQKEDAGQYRCVAKNSLGTAYSKVV | |
| E209) | KLEVE | |
| PR3 I28- | IVGGHEAQPHSRPYMASLQMRGNPGSHFCGGTLIHPSFVLTAAHC | 12 |
| R248 | LRDIPQRLVNVVLGAHNVRTQEPTQQHFSVAQVFLNNYDAENKL | |
| (S195A/I217N/ | NDVLLIQLSSPANLSASVATVQLPQQDQPVPHGTQCLAMGWGRV | |
| G219T) | GAHDPPAQVLQELNVTVVTFFCRPHNICTFVPRRKAGICFGDAGG | |
| | PLICDGIIQGIDSFVNWTCATRLFPDFFTRVALYVDWIRSTLR | |
| AchRa ECD | SEHETRLEAKLFKDYSSVVRPVEDHRQVVEVTVGLQLIQLINVDE | 13 |
| S1-P211 | VNQIVTTNVRLKQQWVDYNLKWNPDDYGGVKKIHIPSEKIWRPD | |
| (V8E/K104N/ | LVLYNNADGDFAIVNFTKVLLQYTGHITWTPPAIFKSYCEIIVTHFP | |
| W149R/V155A/ | FDEQNCSMKLGTRTYDGSAVAINPESDQPDLSNFMESGEWVIKES | |
| C192S/C193S) | RGWKHSVTYSSSPDTPYLDITYHFVMQRLP | |
| BP180 | EEVRKLKARVDELERIRRSILPYGDSMDRIEKDRLQGMAPAAGAD | 14 |
| NC16A | LDKIGLHSDSQEELWMFVRKKLMMEQEN | |
| domain | | |
| E490-N562 | | |
| TSHR ECD | MGCSSPPCECHQEEDFRVTCKDIQRIPSLPPSTQTLKLIETCLRTIPS | 15 |
| M22-L260 | HAFSNLPNISRIYVSIDVTLQQLESHSFYNLSKVTHIEIRNTPNLTYI | |
| JMG55 | DPDALKELPLLKFLGIFNTGLKMFPPLTKVYSTEIFFILEITDNPYM | |
| (H63C/R112P/ | TSIPRNAFQGLCNETLTLKLYNNGFTSVQGYAFNGTKLDAVYLNK | |
| D143P/D151E/ | NKYLTVIDKDAFGGVYSGPSLLDVSQTSVTALPSKGLEHLKELRA | |
| V169R/I253R) | RNTWTL | |

Fusion Protein

| | | |
|---|---|---|
| Musk-D1- | PVITTPLETVDALVEEVATFMCAVESYPQPEISWTRNKILIKLFDTR | 16 |
| 3XG4S-Fc | YSIRENGQLLTILSVEDSDDGIYCCTANNGVGGAVESCGALQVKM | |
| | KIEGRGGGGSGGGGSGGGGSDKTHTCPPCPAPEAAGGPSVFLFPP | |
| | KPKDTLMISRTPEVTCVVVDVSHEDPEVKFNWYVDGVEVHNAKT | |
| | KPREEQYNSTYRVVSVLTVLHQDWLNGKEYKCKVSNKALPAPIE | |
| | KTISKAKGQPREPQVYTLPPSREEMTKNQVSLTCLVKGFYPSDIAV | |
| | EWESNGQPENNYKTTPPVLDSDGSFFLYSKLTVDKSRWQQGNVF | |
| | SCSVMHEALHNHYTQKSLSLSPGK | |
| Musk-DID2- | EKLPKAPVITTPLETVDALVEEVATFMCAVESYPQPEISWTRNKILI | 17 |
| 3XG4S-Fc | KLFDTRYSIRENGQLLTILSVEDSDDGIYCCTANNGVGGAVESCG | |
| | ALQVKMKPKITRPPINVKIIEGLKAVLPCTTMGNPKPSVSWIKGDS | |
| | PLRENSRIAVLESGSLRIHNVQKEDAGQYRCVAKNSLGTAYSKVV | |
| | KLEVEIEGRGGGGSGGGGSGGGGSDKTHTCPPCPAPEAAGGPSVF | |
| | LFPPKPKDTLMISRTPEVTCVVVDVSHEDPEVKFNWYVDGVEVH | |
| | NAKTKPREEQYNSTYRVVSVLTVLHQDWLNGKEYKCKVSNKAL | |
| | PAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQ | |
| | GNVFSCSVMHEALHNHYTQKSLSLSPGK | |
| Musk-DID2- | EKLPKAPVITTPLETVDALVEEVATFMCAVESYPQPEISWTRNKILI | 18 |
| 3XG4S-HSA | KLFDTRYSIRENGQLLTILSVEDSDDGIYCCTANNGVGGAVESCG | |
| | ALQVKMKPKITRPPINVKIIEGLKAVLPCTTMGNPKPSVSWIKGDS | |
| | PLRENSRIAVLESGSLRIHNVQKEDAGQYRCVAKNSLGTAYSKVV | |
| | KLEVEGGGGSGGGGSGGGGSDAHKSEVAHRFKDLGEENFKALVL | |
| | IAFAQYLQQCPFEDHVKLVNEVTEFAKTCVADESAENCDKSLHTL | |
| | FGDKLCTVATLRETYGEMADCCAKQEPERNECFLQHKDDNPNLP | |
| | RLVRPEVDVMCTAFHDNEETFLKKYLYEIARRHPYFYAPELLFFA | |
| | KRYKAAFTECCQAADKAACLLPKLDELRDEGKASSAKQRLKCAS | |
| | LQKFGERAFKAWAVARLSQRFPKAEFAEVSKLVTDLTKVHTECC | |
| | HGDLLECADDRADLAKYICENQDSISSKLKECCEKPLLEKSHCIAE | |
| | VENDEMPADLPSLAADFVESKDVCKNYAEAKDVFLGMFLYEYA | |
| | RRHPDYSVVLLLRLAKTYETTLEKCCAAADPHECYAKVFDEFKPL | |
| | VEEPQNLIKQNCELFEQLGEYKFQNALLVRYTKKVPQVSTPTLVE | |
| | VSRNLGKVGSKCCKHPEAKRMPCAEDYLSVVLNQLCVLHEKTPV | |
| | SDRVTKCCTESLVNRRPCFSALEVDETYVPKEFNAETFTFHADICT | |
| | LSEKERQIKKQTALVELVKHKPKATKEQLKAVMDDFAAFVEKCC | |
| | KADDKETCFAEEGKKLVAASQAALGL | |
| PR3(S195A/ | IVGGHEAQPHSRPYMASLQMRGNPGSHFCGGTLIHPSFVLTAAHC | 19 |
| I217N/G219T)- | LRDIPQRLVNVVLGAHNVRTQEPTQQHFSVAQVFLNNYDAENKL | |
| 3XG4S- | NDVLLIQLSSPANLSASVATVQLPQQDQPVPHGTQCLAMGWGRV | |
| HSA-6his | GAHDPPAQVLQELNVTVVTFFCRPHNICTFVPRRKAGICFGDAGG | |
| | PLICDGIIQGIDSFVNWTCATRLFPDFFTRVALYVDWIRSTLRGGG | |
| | GSGGGGSGGGGSDAHKSEVAHRFKDLGEENFKALVLIAFAQYLQ | |
| | QCPFEDHVKLVNEVTEFAKTCVADESAENCDKSLHTLFGDKLCT | |
| | VATLRETYGEMADCCAKQEPERNECFLQHKDDNPNLPRLVRPEV | |
| | DVMCTAFHDNEETFLKKYLYEIARRHPYFYAPELLFFAKRYKAAF | |
| | TECCQAADKAACLLPKLDELRDEGKASSAKQRLKCASLQKFGER | |
| | AFKAWAVARLSQRFPKAEFAEVSKLVTDLTKVHTECCHGDLLEC | |
| | ADDRADLAKYICENQDSISSKLKECCEKPLLEKSHCIAEVENDEMP | |
| | ADLPSLAADFVESKDVCKNYAEAKDVFLGMFLYEYARRHPDYSV | |
| | VLLLRLAKTYETTLEKCCAAADPHECYAKVFDEFKPLVEEPQNLI | |
| | KQNCELFEQLGEYKFQNALLVRYTKKVPQVSTPTLVEVSRNLGK | |
| | VGSKCCKHPEAKRMPCAEDYLSVVLNQLCVLHEKTPVSDRVTKC | |
| | CTESLVNRRPCFSALEVDETYVPKEFNAETFTFHADICTLSEKERQI | |
| | KKQTALVELVKHKPKATKEQLKAVMDDFAAFVEKCCKADDKET | |
| | CFAEEGKKLVAASQAALGLGGHHHHHH | |
| AchRa(V8E/ | SEHETRLEAKLFKDYSSVVRPVEDHRQVVEVTVGLQLIQLINVDE | 20 |
| K104N/S143T/ | VNQIVTTNVRLKQQWVDYNLKWNPDDYGGVKKIHIPSEKIWRPD | |
| W149R/V155A/ | LVLYNNADGDFAIVNFTKVLLQYTGHITWTPPAIFKSYCEIIVTHFP | |
| C192S/C193S)- | FDEQNCTMKLGTRTYDGSAVAINPESDQPDLSNFMESGEWVIKES | |
| 3XG4S-HSA | RGWKHSVTYSSSPDTPYLDITYHFVMQRLPGGGGSGGGGSGGGG | |
| | SDAHKSEVAHRFKDLGEENFKALVLIAFAQYLQQCPFEDHVKLV | |
| | NEVTEFAKTCVADESAENCDKSLHTLFGDKLCTVATLRETYGEM | |
| | ADCCAKQEPERNECFLQHKDDNPNLPRLVRPEVDVMCTAFHDNE | |
| | ETFLKKYLYEIARRHPYFYAPELLFFAKRYKAAFTECCQAADKAA | |
| | CLLPKLDELRDEGKASSAKQRLKCASLQKFGERAFKAWAVARLS | |
| | QRFPKAEFAEVSKLVTDLTKVHTECCHGDLLECADDRADLAKYI | |
| | CENQDSISSKLKECCEKPLLEKSHCIAEVENDEMPADLPSLAADFV | |
| | ESKDVCKNYAEAKDVFLGMFLYEYARRHPDYSVVLLLRLAKTYE | |
| | TTLEKCCAAADPHECYAKVFDEFKPLVEEPQNLIKQNCELFEQLG | |
| | EYKFQNALLVRYTKKVPQVSTPTLVEVSRNLGKVGSKCCKHPEA | |
| | KRMPCAEDYLSVVLNQLCVLHEKTPVSDRVTKCCTESLVNRRPC | |
| | FSALEVDETYVPKEFNAETFTFHADICTLSEKERQIKKQTALVELV | |
| | KHKPKATKEQLKAVMDDFAAFVEKCCKADDKETCFAEEGKKLV | |
| | AASQAALGL | |
| Fc-6XG4S- | MDKTHTCPPCPAPELLGGPSVFLFPPKPKDTLMISRTPEVTCVVVD | |
| Fc-G4S-BP180 | VSHEDPEVKFNWYVDGVEVHNAKTKPCEEQYGSTYRCVSVLTVL | |
| | HQDWLNGKEYKCKVSNKALPAPIEKTISKAKGQPREPQVYTLPPS | |
| | REEMTKNQVSLTCLVKGFYPSDIAVEWESNGQPENNYKTTPPVLD | |
| | SDGSFFLYSKLTVDKSRWQQGNVFSCSVMHEALHNHYTQKSLSL | |
| | SPGKGGGGSGGGGSGGGGSGGGGSGGGGSGGGGSDKTHTCPPCP | 21 |
| | APELLGGPSVFLFPPKPKDTLMISRTPEVTCVVVDVSHEDPEVKFN | |
| | WYVDGVEVHNAKTKPCEEQYGSTYRCVSVLTVLHQDWLNGKEY | |
| | KCKVSNKALPAPIEKTISKAKGQPREPQVYTLPPSREEMTKNQVSL | |
| | TCLVKGFYPSDIAVEWESNGQPENNYKTTPPVLDSDGSFFLYSKL | |
| | TVDKSRWQQGNVFSCSVMHEALHNHYTQKSLSLSPGKGGGGSEE | |
| | VRKLKARVDELERIRRSILPYGDSMDRIEKDRLQGMAPAAGADLD | |
| | KIGLHSDSQEELWMFVRKKLMMEQEN | |
| TSHR- | GCSSPPCECHQEEDFRVTCKDIQRIPSLPPSTQTLKLIETCLRTIPSH | |
| JMG55- | AFSNLPNISRIYVSIDVTLQQLESHSFYNLSKVTHIEIRNTPNLTYID | |
| (H63C/R112P/ | PDALKELPLLKFLGIFNTGLKMFPPLTKVYSTEIFFILEITDNPYMTS | |
| D143P/D151E/ | IPRNAFQGLCNETLTLKLYNNGFTSVQGYAFNGTKLDAVYLNKN | 22 |
| V169R/I253R)- | KYLTVIDKDAFGGVYSGPSLLDVSQTSVTALPSKGLEHLKELRAR | |
| 3XG4S-Fc | NTWTLGGGGSGGGGSGGGGSDKTHTCPPCPAPEAAGGPSVFLFPP | |
| | KPKDTLMISRTPEVTCVVVDVSHEDPEVKFNWYVDGVEVHNAKT | |
| | KPREEQYNSTYRVVSVLTVLHQDWLNGKEYKCKVSNKALPAPIE | |
| | KTISKAKGQPREPQVYTLPPSREEMTKNQVSLTCLVKGFYPSDIAV | |
| | EWESNGQPENNYKTTPPVLDSDGSFFLYSKLTVDKSRWQQGNVF | |
| | SCSVMHEALHNHYTQKSLSLSPGK | |
| TSHR- | MGCSSPPCECHQEEDFRVTCKDIQRIPSLPPSTQTLKLIETCLRTIPS | 23 |
| JMG55- | HAFSNLPNISRIYVSIDVTLQQLESHSFYNLSKVTHIEIRNTPNLTYI | |
| (H63C/R112P/ | DPDALKELPLLKFLGIFNTGLKMFPPLTKVYSTEIFFILEITDNPYM | |
| D143P/D151E/ | TSIPRNAFQGLCNETLTLKLYNNGFTSVQGYAFNGTKLDAVYLNK | |
| V169R/I253R)- | NKYLTVIDKDAFGGVYSGPSLLDVSQTSVTALPSKGLEHLKELRA | |
| 3XG4S- | RNTWTLIEGRGGGGSGGGGSGGGGSDAHKSEVAHRFKDLGEENF | |
| HSA-6his | KALVLIAFAQYLQQCPFEDHVKLVNEVTEFAKTCVADESAENCD | |
| | KSLHTLFGDKLCTVATLRETYGEMADCCAKQEPERNECFLQHKD | |
| | DNPNLPRLVRPEVDVMCTAFHDNEETFLKKYLYEIARRHPYFYAP | |
| | ELLFFAKRYKAAFTECCQAADKAACLLPKLDELRDEGKASSAKQ | |
| | RLKCASLQKFGERAFKAWAVARLSQRFPKAEFAEVSKLVTDLTK | |
| | VHTECCHGDLLECADDRADLAKYICENQDSISSKLKECCEKPLLE | |
| | KSHCIAEVENDEMPADLPSLAADFVESKDVCKNYAEAKDVFLGM | |
| | FLYEYARRHPDYSVVLLLRLAKTYETTLEKCCAAADPHECYAKV | |
| | FDEFKPLVEEPQNLIKQNCELFEQLGEYKFQNALLVRYTKKVPQV | |
| | STPTLVEVSRNLGKVGSKCCKHPEAKRMPCAEDYLSVVLNQLCV | |
| | LHEKTPVSDRVTKCCTESLVNRRPCFSALEVDETYVPKEFNAETFT | |
| | FHADICTLSEKERQIKKQTALVELVKHKPKATKEQLKAVMDDFA | |
| | AFVEKCCKADDKETCFAEEGKKLVAASQAALGLGGHHHHHH | |

### Example 3. Conjugation of Protein Targets

Protein targets were concentrated to 10 mg/mL using a Amicon Ultra Concentator with 30K MWCO. The protein sample was then buffer exchanged into 100 mM Borate Buffer (pH 8.3), 50 mM Sodium Chloride and 1 mM EDTA using a 40K MWCO Zeba spin column. 6.5 molar equivalents of linker/payload was added to the protein solution and incubated at 25 C for 3 hours, followed by purification on an AKTA FPLC purification system containing a Superdex 200 column (26/60) pre-equilibrated in 1×PBS (pH 7.4). Fractions containing the conjugated protein were pooled and filtered using a 0.2 micron syringe filter (PES membrane).

### Example 4: MuSK Conjugate-Mediated Internalization, Degradation and In Vivo Serum Clearance of Patient-Derived Pathogenic Antibodies

In this example, conjugates targeted to MuSK antibodies (MuSK conjugates) were tested for the ability to mediate cellular uptake and degradation of patient-derived autoantibodies and tested in mouse for the ability to clear autoantibodies from serum.

Conjugate-mediated cellular uptake of patient-derived antibodies was tested in the Hep G2 human hepatocellular carcinoma cell line. 200,000 wild type or ASGPR ½ knock out Hep G2 cells were seeded into 24-well plates and incubated at 37° C. for 48-72 hr. Conjugates or unconjugated proteins were mixed in equimolar ratios with a patient-derived autoantibody labeled with Alexa Fluor 488 or Alexa Fluor 647 and serial diluted in 3-fold steps before incubating at 22° C. After 30 minutes, media was removed from the cells and the conjugate or protein with antibody solutions were added to the cells and incubated for 2 hr at 37° C. Cells were then washed with phosphate buffered saline, lifted with TrypLE and analyzed for fluorescence signal by flow cytometry.

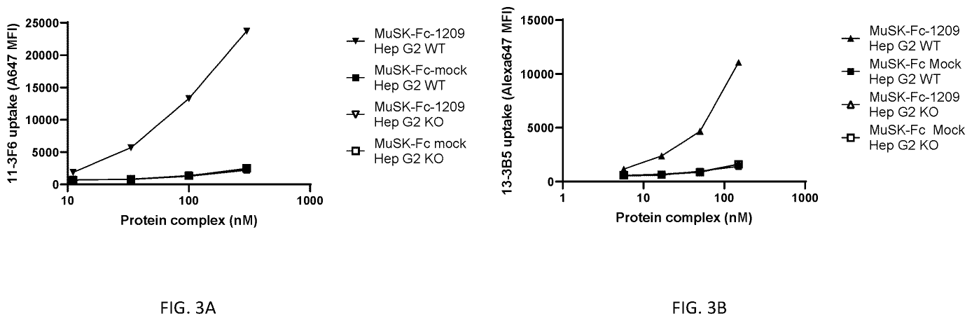

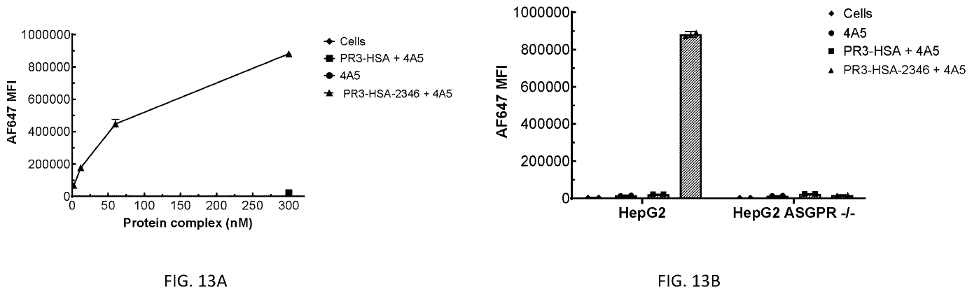

FIGS. 3A-3B show MuSK conjugate-mediated cellular uptake of two patient-derived pathogenic antibodies 11-3F6 (FIG. 3A) and 13-3B5 (FIG. 3B) (Huijbers M, et al, Neurol Neuroimmunol Neuroinflamm, 2019). Activity was tested with four treatment groups and is presented as mean fluorescence intensity (MFI) vs. protein/conjugate-antibody complex concentration. In these studies, the MuSK Ig1 domain (SEQ ID NO: 10) was produced as a fusion with an Fc carrier polypeptide (SEQ ID NO: 3) to provide a MuSK-Fc fusion protein (SEQ ID NO: 16) and conjugated to the 1209 tri-GalNAc or mock conjugated. The treatment groups were as follows: group 1) MuSK-Fc-1209 and antibody in wild type (WT) HepG2 cells, group 2) MuSK-Fc mock and antibody in wild type HepG2 cells, group 3) MuSK-Fc-1209 and antibody in ASGPR ½ knockout HepG2 cells (KO), group 4) MuSK-Fc mock and antibody in ASGPR ½ knockout HepG2 cells. A concentration-dependent increase in fluorescence signal was observed for both 11-3F6 and 13-3B5 antibodies in the presence of the MuSK conjugate, while low fluorescence signal was observed with the MuSK-Fc mock in WT HepG2 cells or either construct in KO cells. These results demonstrate that antibody uptake is dependent on the ASGPR internalizing receptor and the MuSK-Fc-ASGPR ligand conjugate.

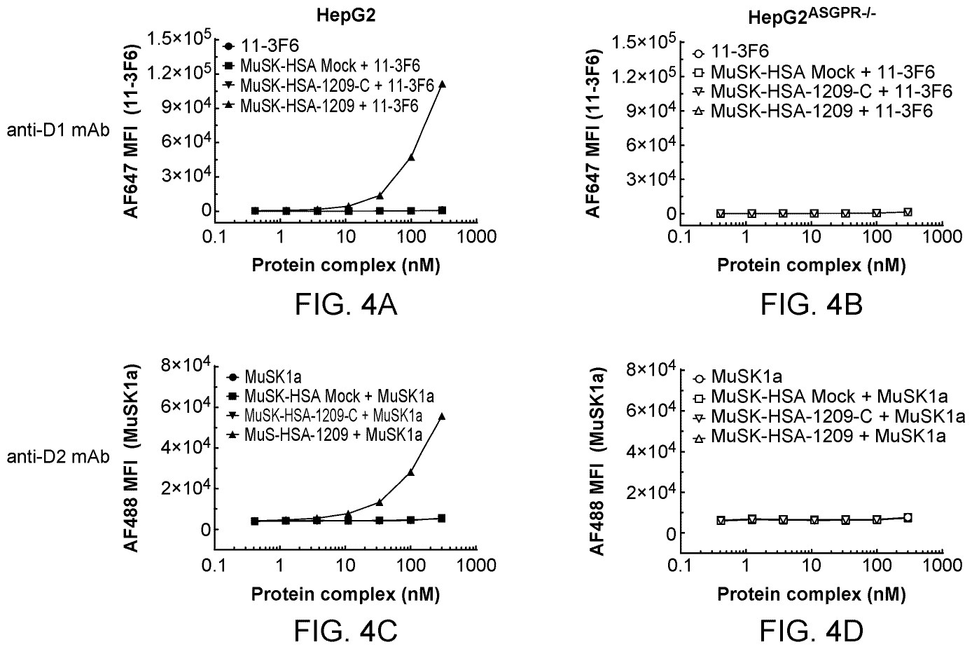

FIGS. 4A-4D illustrate MuSK-HSA polypeptide fusion conjugates mediating patient-derived, MuSK-binding antibody cellular uptake. In these studies, the MuSK Ig1 and Ig2 domains (SEQ ID NO: 11) were produced as a fusion with an HSA carrier polypeptide (SEQ ID NO: 1) to provide a MuSK-HSA fusion protein (SEQ ID NO: 18) and conjugated to the 1209 tri-GalNAc, 1209-C, the enantiomer GalNAc of 1209 with low ASGPR binding, or mock conjugated. WT Hep G2 (FIGS. 4A, 4C) or ASGPR ½ KO Hep G2 (FIGS. 4B, 4D) cells were tested with four different treatment groups: group 1) antibody alone, group 2) MuSK-HSA mock with antibody, group 3) MuSK-HSA-1209-C with antibody, group 4) MuSK-HSA-1209 with antibody. The antibodies tested in this study were the MuSK Ig1 domain binding 11-3F6 (FIGS. 4A, 4B) and the MuSK Ig2 domain binding antibody MuSK1a (Takata K, et al, JCI Insight, 2019) (FIGS. 4C, 4D). Data is presented as mean Alexa Fluor 488 of Alexa Fluor 647 fluorescence intensity vs. protein/conjugate-antibody complex concentration. Concentration-dependent antibody uptake was observed for both 11-3F6 and MuSK1a in the presence of MuSK-HSA-1209 in WT HepG2 cells, but not in HepG2 ASGPR ½ KO cells. Neither MuSK-HSA mock or MuSK-HSA-1209-C were capable of inducing antibody uptake. The data indicate that MuSK conjugates mediated antibody uptake in an ASGPR-dependent manner.

The ability of MuSK conjugates to mediate cellular uptake and lysosomal delivery of patient-derived antibodies was tested in the Hep G2 human hepatocellular carcinoma cell line is antibodies labeled with the pH-sensitive dye pHrodo green. An increase in fluorescence is observed with a decrease in pH such as upon accumulation in the lysosome, where protein degradation occurs. To test conjugate-dependent lysosomal delivery of antibodies, 50,000 wild type or ASGPR % knock out Hep G2 cells were seeded into 96-well plates and incubated at 37° C. for 48 hr. conjugates or unconjugated proteins were mixed in equimolar ratios with a patient-derived autoantibody labeled with pHrodo green and serial diluted in 3-fold steps before incubating at 22° C. After 30 minutes, media was removed from the cells and the conjugate or protein with antibody solutions were added to the cells and incubated for 24 hr at 37° C. Cells were then washed with phosphate buffered saline, lifted with TrypLE and analyzed for fluorescence signal by flow cytometry.

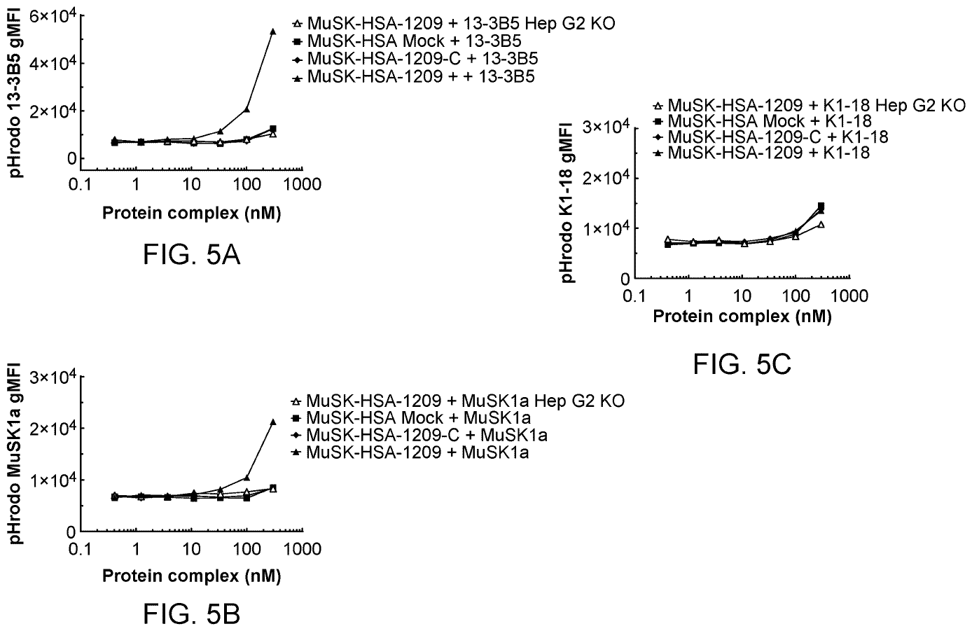

FIGS. 5A-5C show the ability of MuSK conjugates to mediate lysosomal delivery of patient-derived pathogenic anti-MuSK antibodies. The MuSK Ig1 and Ig2 domains fused to HSA, the MuSK-HSA fusion protein (SEQ ID NO: 18) were conjugated with the 1209 tri-GalNAc, 1209-C, the enantiomer GalNAc of 1209 with low ASGPR binding, or mock conjugated. Activity with the anti-MuSK antibodies 13-3B5 (FIG. 5A) and MuSK1a (FIG. 5B) or the anti-TSHR antibody K1-18 (FIG. 5C) was tested in WT HepG2 cells. Activity with MuSK-HSA-1209 was also tested in ASGPR ½ KO Hep G2 cells. Treatment groups were as follows: group 1) MuSK-HSA-1209 with antibody in ASGPR ½ KO Hep G2 cells, group 2) MuSK-HSA mock with antibody in WT Hep G2 cells, group 3) MuSK-HSA-1209-C with antibody in WT Hep G2 cells, group 4) MuSK-HSA-1209 in WT Hep G2 cells. MuSK-HSA-1209 induced a concentration-dependent increase in fluorescence with either 13-3B5 or MuSK1a but not K1-18, which does not bind to MuSK, in WT Hep G2 cells. The signal change was dependent on ASGPR, as little increase in signal was observed in ASGPR ½ KO Hep G2 cells. MuSK-HSA mock and MuSK-HSA-1209-C caused minimal increase in signal. Combined with the above antibody uptake results, the results demonstrate the ability of MuSK conjugates to cause selective cellular uptake and lysosomal delivery of patient-derived antibodies.

Conjugate activity was tested in mice with the patient-derived 13-3B5 antibody. Female C57BL/6 mice were injected intravenously with 1 mg/kg 13-3B5 labeled with biotin to enable detection by sandwich ELISA. 16 hr later, a blood sample was collected from each animal. Treatments were then administered intravenously. Blood samples were collected at 1, 4, 24 and 48 hr post treatment and serum was obtained using serum separator tubes (Beckton Dickinson). Antibody levels were measured using a sandwich ELISA method with a goat anti-human Fc capture antibody (Invitrogen) and streptavidin-horseradish peroxidase detection (Pierce). This method was optimized to prevent interference from MuSK protein or MuSK conjugate in the serum. Results are presented as the biotin-13-3B5 concentration in ng/ml or % starting biotin-13-3B5 as a function of time (hr) post treatment dose.

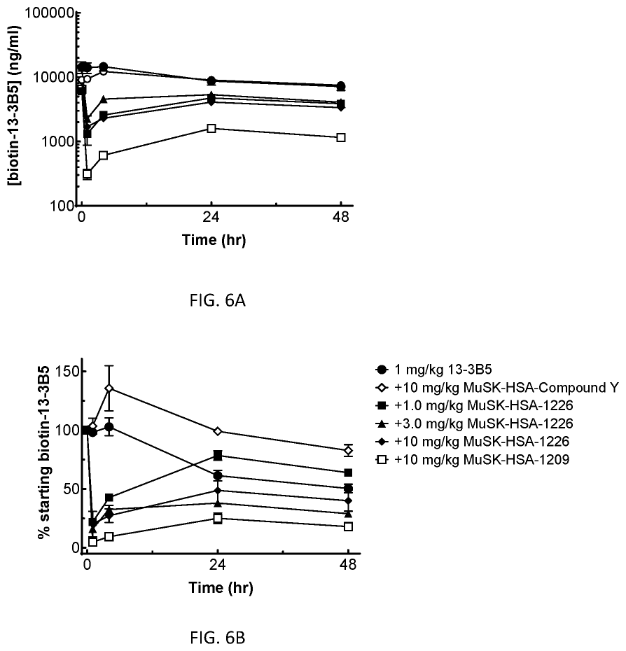

FIGS. 6A-6B show the ability of conjugates to clear the patient-derived antibody 13-3B5 from mouse serum. Mice were injected with biotin-13-3B5 then treated with conjugates, or left untreated as a control. The treatment groups consisted of group 1) no treatment, group 2) 10 mg/kg MuSK-HSA-Compound Y, a non-ASGPR binding enantiomer of 1226, group 3) 1.0 mg/kg MuSK-HSA-1226, group 4) 3.0 mg/kg MuSK-HSA-1226, group 5) 10 mg/kg MuSK-HSA-1226, and group 6) 10 mg/kg MuSK-HSA-1209. MuSK-HSA-1226 or MuSK-HSA-1209 caused rapid depletion of biotin-13-3B5, as compared to the untreated group, evident at 1 hr post dose. In contrast, MuSK-HSA-Compound Y stabilized Ab levels relative to the untreated control. 4 hr post treatment, antibody levels in the 10 mg/kg MuSK-HSA-Compound Y. 10 mg/kg MuSK-HSA-1226, 10 mg/kg, MuSK-HSA-1209 and untreated groups were 136%, 27.3%, 9.4 and 103% starting levels, respectively. These results demonstrate that conjugates can clear the patient-derived antibody 13-3B5 in a dose-dependent manner in mice.

### Example 5: Internalization, Degradation and In Vivo Serum Clearance of Autoantibodies Facilitated by Target Binding Conjugates with TSHR-HSA and TSHR-Fc Configuration

This example was designed to evaluate the ability of stabilized TSHR-based conjugates fused to human serum albumin (comprising an HSA carrier polypeptide) and Fe (comprising an Fc carrier polypeptide) (see FIG. 2) (conjugates targeted to TSHR antibodies) to mediate cellular internalization and degradation and in vivo clearance of autoantibodies.

To investigate the ability of target binding conjugates described herein to internalize and uptake Graves' patient-derived autoantibodies in human cells a cellular uptake assay was performed. 50,000 HepG2 cells were seeded onto 96 well plates and incubated at 37 degrees Celsius for 48 hrs. TSHR-based conjugates were mixed with equimolar concentrations of patient-derived autoantibodies and incubated for 30 mins at 22 degrees Celsius. A log 3 seral dilution was performed covering a final concentration range from 150-5.67 nM. Experimental groups were treated for 2 hrs with test articles as described below. Following incubation, the cells were washed with phosphate buffered saline, detached using 0.25% trypsin solution and analyzed by flow cytometry.

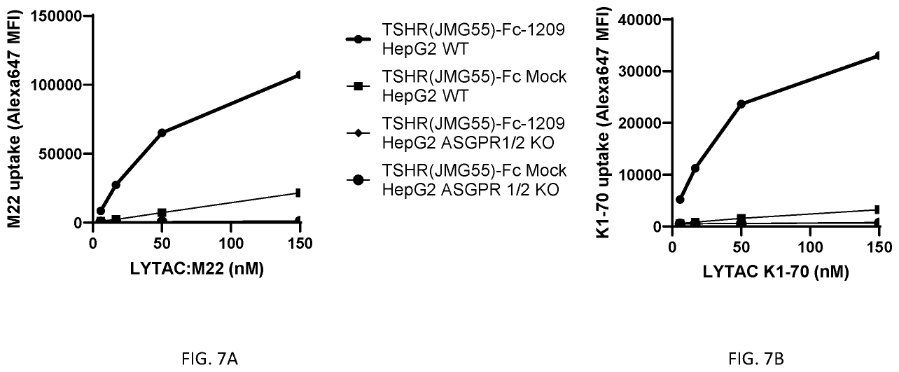

FIGS. 7A-7B illustrate conjugate-mediated in vitro uptake of two patient-derived autoantibodies, M22 (Sanders, Lancet, 2003) and K1-70. (Evans, Clin Endocrinol. 2008) in human hepatocellular carcinoma (HepG2) wild-type (WT) or ASGPR½ knockout (KO) cells. In these studies, thermostable TSHR, JMG55 (Miller-Gallacher, J Mol Endocrinol, 2009) (SEQ ID NO: 15), fused to an Fc carrier polypeptide (SEQ ID NO: 3) as described herein to provide a TSHR-Fc fusion protein (SEQ ID NO: 22) and conjugated to 1209, or mock conjugated. Shown are autoantibody uptake in human HepG2 cells in four treatment groups: (Group 1) TSHR-Fc-1209 in HepG2 WT cells; (Group 2) TSHR-Fc mock conjugated in HepG2 WT cells; (Group 3) TSHR-Fc-1209 in HepG2 ASGPR½ KO; and (Group 4) TSHR-Fc mock conjugated in HepG2 ASGPR½ KO. FIG. 7A shows treatment with Group 1 resulted in significant autoantibody uptake activity of M22 conjugated to AlexaFluor647 in HepG2 cells and low or minimal autoantibody uptake activity in cells with treatment Group 2. No autoantibody uptake activity was detected in treatment Groups 3 and 4. Autoantibody uptake activity was measured by flow cytometry in the APC channel and is indicated by mean fluorescence intensity (MFI) on the y-axis. The x-axis indicates concentration of TSHR-Fc-based conjugate: M22-Alexa647 (nM). FIG. 7B shows treatment with Group 1 resulted in significant autoantibody uptake activity of K1-70 autoantibody in HepG2 cells and low or minimal autoantibody uptake activity in cells with treatment Groups 2-4. Autoantibody uptake activity was measured by flow cytometry in the APC channel and is indicated by mean fluorescence intensity (MFI) on the y-axis. The x-axis indicates concentration of TSHR-Fc-based conjugate: K1-70-Alexa647 (nM). The results show that TSHR-based conjugates mediate uptake of Graves' patient-derived autoantibodies in human cells. The data demonstrate that uptake of autoantibodies with TSHR-based conjugates is ASGPR-dependent.

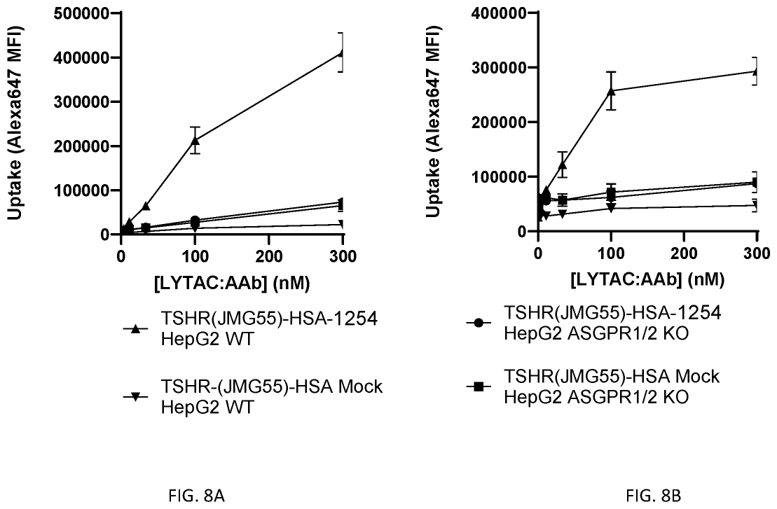

FIGS. 8A-8B illustrate uptake of patient-derived autoantibodies using thermostable TSHR variant-based target binding conjugates fused to HSA as described herein in human HepG2 cells. In these studies, thermostable TSHR, JMG55 (Miller-Gallacher, J Mol Endocrinol, 2009) (SEQ ID NO: 15), fused to an HSA carrier polypeptide with a poly-his tag (SEQ ID NO: 2) as described herein to provide a TSHR-HSA fusion protein (SEQ ID NO: 23) and conjugated to 1254, or mock conjugated. Shown are the results of autoantibody uptake assays, performed as described above, in four treatment groups: (Group 1) TSHR-HSA-1254 in HepG2 WT; (Group 2) TSHR-HSA mock conjugated in HepG2 WT; (Group 3) TSHR-HSA-1254 in HepG2 ASGPR½ KO; and (Group 4) TSHR-HSA mock conjugated in HepG2 ASGPR½ KO. Uptake of two patient-derived autoantibodies was indicated by quantification of Alexa647 in MFI (y-axis). FIG. 8A shows treatment with Group 1 resulted in significant autoantibody uptake activity of M22 conjugated to AlexaFluor647 in HepG2 cells and low or minimal autoantibody uptake activities in the other treatment groups. Autoantibody uptake activity was measured by flow cytometry in the APC channel and is indicated by mean fluorescence intensity (MFI) on the y-axis. The x-axis indicates concentration of TSHR-HSA-based conjugate: M22-Alexa647 (nM). FIG. 8B shows treatment with Group 1 resulted in significant autoantibody uptake activity of K1-18 (Evans, Clin Endocrinol, 2008) autoantibody in HepG2 cells and low or minimal autoantibody uptake activity in cells with treatment Groups 2-4. Autoantibody uptake activity was measured by flow cytometry in the APC channel and is indicated by mean fluorescence intensity (MFI) on the y-axis. The x-axis indicates concentration of TSHR-HSA-based conjugate: K1-18-Alexa647 (nM). The data demonstrate that TSHR-HSA-based target binding conjugate is effective in internalizing patient-derived autoantibody (e.g., M22 and K1-18) in human cells.

### Example 6. AChR Conjugate-Mediated Internalization and Degradation of Patient-Derived Pathogenic Antibodies

In this example, an acetylcholine receptor (AChR) conjugate based on the extracellular portion of the AChRα subunit fused to an HSA polypeptide (a conjugate targeted to AChR antibodies) was evaluated for the ability to mediate pathogenic antibody uptake and degradation in cells. The AChR protein contained stabilizing mutations described herein.

Conjugate-mediated cellular uptake of patient-derived antibodies was tested in the Hep G2 human hepatocellular carcinoma cell line. 200,000 wild type or ASGPR ½ knock out Hep G2 cells were seeded into 24-well plates and incubated at 37° C. for 48 hr. Conjugates or unconjugated proteins were mixed in equimolar ratios with a patient-derived autoantibody labeled with Alexa Fluor 647 and serial diluted in 5-fold steps from 300 nM to 12 nM before incubating at 22° C. After 30 minutes, media was removed from the cells and the conjugate or protein with antibody solutions were added to the cells and incubated for 2 hr at 37° C. Cells were then washed with phosphate buffered saline, lifted with TrypLE and analyzed for fluorescence signal by flow cytometry.

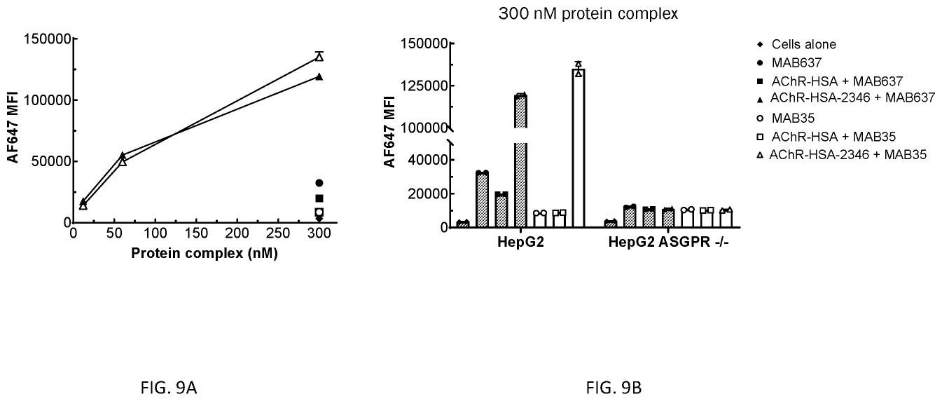

FIGS. 9A-B show AChR conjugate-mediated cellular uptake of the patient-derived antibody MAB637 (Graus Y et al, J Immunol, 1997) and the pathogenic tool antibody MAB35 (Tzartos S. et al, FEBS. 1983: Loutrari H, et al, Eur J Immunol, 1992). Activity was tested with three groups for each antibody and compared to signal from cells alone in wild type (WT) Hep G2 cells or Hep G2 ASGPR½ knockout (KO) cells. Results are presented as mean fluorescence intensity (MFI) vs. protein/conjugate-antibody complex concentration. The stabilized AChRα subunit (SEQ ID NO: 13) was produced as a fusion with an HSA carrier polypeptide (SEQ ID NO: 1) to provide a AChR-HSA fusion protein (SEQ ID NO: 20) and conjugated to 2346. The treatment groups for each antibody were as follows: group 1) antibody alone, group 2) AChR-HSA with antibody, group 3) AChR-HSA-2346 with antibody. A concentration-dependent increase in fluorescence signal was observed for both MAB637 and MAB35 in the presence of the AChR conjugate, while low fluorescence signal was observed with either antibody alone or in the presence of unconjugated AChR-HSA. Significantly lower fluorescence signal was observed with any treatment condition in HepG2 ASGPR½ KO cells. These results demonstrate that antibody uptake is dependent on the ASGPR internalizing receptor and the AChR-HSA-ASGPR ligand conjugate.

Conjugate-mediated antibody degradation was monitored indirectly using a labeled goat anti-human IgG F(ab′)₂ fragment (Jackson ImmunoResearch) probe. The F(ab′)₂ fragment was dual labeled with TAMRA and the QSY7 quencher as described (Wen Y, et al, American Assoc Pharm Sci J, 2020). Upon degradation of the probe, an increase in TAMRA fluorescence is observed. 200,000 wild type Hep G2 cells were seeded into 24-well plates and incubated at 37° C. for 48 hr. Conjugates or unconjugated proteins were mixed in equimolar ratios with unlabeled antibodies and the degradation probe giving a final concentration of 50 nM of each component in complete growth media. After 30 minutes, media was removed from the cells and the treatment solutions were added to the cells. Fluorescence was monitored every hour for 98 hr using an Incucyte S3 imaging system (Sartorius). Each condition was tested in duplicate, and the integrated fluorescence intensity from 3 images in each well were used to generate an average integrated intensity at each time point.

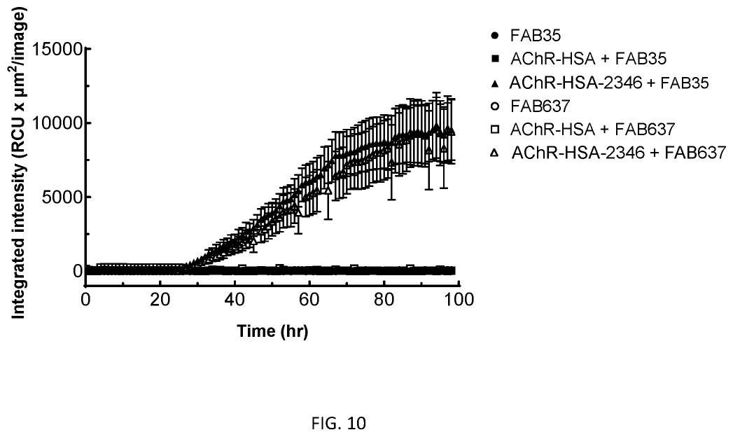

FIG. 10 illustrates the ability of an AChR conjugate to induce degradation of pathogenic antibodies in Hep G2 cells. Six treatment groups were tested: group 1) MAB35, group 2) AChR-HSA with MAB35, group 3) AChR-HSA-2346 with MAB35, group 4) MAB637, group 5) MAB637 with AChR-HSA and group 6) AChR-HSA-2346 with MAB637. All groups also contained the anti-human IgG F(ab′)₂ probe labeled with TAMRA and QSY7 that binds to the pathogenic antibodies. In the groups containing AChR-HSA-2346 and either MAB35 or MAB637, an increase in fluorescence occurs beginning about 20 hrs after treatment and continues rapidly for about 50 hrs before plateauing. In contrast, groups containing either antibody alone or antibody with AChR-HSA show minimal increase in signal over the course of the experiment. At 98 hrs, the AChR-HSA-2346 groups showed a 500-fold and a 201-fold increase in fluorescence vs antibody alone for MAB35 and MAB637, respectively. These results demonstrate the ability of an AChR conjugate to mediate degradation of pathogenic antibodies in a cellular assay.

### Example 6. BP180 Conjugate-Mediated Internalization and Degradation of a Patient-Derived Antibody

In this example, a BP180 conjugate based on the main immunogenic region of BP180 (NC16A domain) fused to an Fc polypeptide (a conjugate targeted to BP180 antibodies) was evaluated for the ability to mediate pathogenic antibody uptake and degradation in cells. The BP180 protein construct is described herein.

Conjugate-mediated cellular uptake of a patient-derived antibody was tested in the Hep G2 human hepatocellular carcinoma cell line. 200,000 wild type or ASGPR ½ knock out Hep G2 cells were seeded into 24-well plates and incubated at 37° C. for 48 hr. Conjugates or unconjugated proteins were mixed in equimolar ratios with a patient-derived autoantibody labeled with Alexa Fluor 647 and serial diluted in 5-fold steps from 300 nM to 2.4 nM before incubating at 22° C. After 30 minutes, media was removed from the cells and the conjugate or protein with antibody solutions were added to the cells and incubated for 2 hr at 37° C. Cells were then washed with phosphate buffered saline, lifted with TrypLE and analyzed for fluorescence signal by flow cytometry.

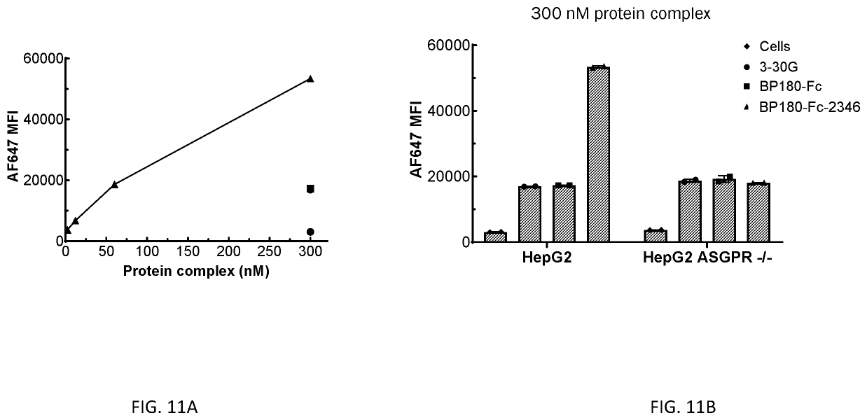

FIGS. 11A-B show BP180 conjugate-mediated cellular uptake of the patient-derived antibody 3-30G (WO2020072937A1). Activity was tested with three groups and compared to signal from cells alone in wild type (WT) Hep G2 cells or Hep G2 ASGPR½ knockout (KO) cells. Results are presented as mean fluorescence intensity (MFI) vs. protein/conjugate-antibody complex concentration. The stabilized BP180 (SEQ ID NO: 14) was produced as a fusion with an scFc carrier polypeptide (SEQ ID NO: 4) to provide a BP180-Fc fusion protein (SEQ ID NO: 21) and conjugated to 2346. The treatment groups were as follows: group 1) antibody alone, group 2) BP180-Fc with antibody, group 3) BP180-Fc-2346 with antibody. A concentration-dependent increase in fluorescence signal was observed for 3-30G in the presence of the BP180 conjugate, while lower fluorescence signal was observed with antibody alone or in the presence of unconjugated BP180-Fc. Significantly lower fluorescence signal was observed with any treatment condition in HepG2 ASGPR½ KO cells. These results demonstrate that antibody uptake is dependent on the ASGPR internalizing receptor and the BP180-Fc-GalNAc conjugate.

Conjugate-mediated antibody degradation was monitored indirectly using a labeled goat anti-human IgG F(ab′)₂ fragment (Jackson ImmunoResearch) probe. The F(ab′)₂ fragment was dual labeled with TAMRA and the QSY7 quencher as described (Wen Y, et al, American Assoc Pharm Sci J. 2020). Upon degradation of the probe, an increase in TAMRA fluorescence is observed. 200.000 wild type Hep G2 cells were seeded into 24-well plates and incubated at 37° C. for 48 hr. Conjugates or unconjugated proteins were mixed in equimolar ratios with unlabeled antibodies and the degradation probe giving a final concentration of 50 nM of each component in complete growth media. After 30 minutes, media was removed from the cells and the treatment solutions were added to the cells. Fluorescence was monitored every hour for 98 hr using an Incucyte S3 imaging system (Sartorius). Each condition was tested in duplicate, and the integrated fluorescence intensity from 3 images in each well were used to generate an average integrated intensity at each time point.

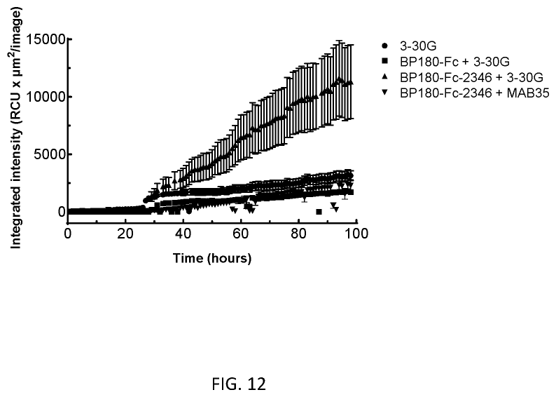

FIG. 12 illustrates the ability of a BP180 conjugate to induce degradation of the pathogenic antibody 3-30G in Hep G2 cells. Four treatment groups were tested: group 1) 3-30G, group 2) BP180-Fc with 3-30G, group 3) BP180-Fc-2346 with 3-30G and group 4) BP180-Fc-2346 with MAB35 (anti-AChR antibody). All groups also contained the anti-human IgG F(ab′)₂ probe labeled with TAMRA and QSY7 that binds to the pathogenic antibodies. In the group containing BP180-Fc-2346 and 3-30G, an increase in fluorescence occurs beginning ~20 hr after treatment and continues until the last time point at 98 hr. In contrast, groups containing either antibody alone or antibody with BP180-Fc show minimal increase in signal over the course of the experiment. Antibody degradation is selective, as minimal signal increase is observed in the group containing BP180-Fc-2346 and MAB35, which does not bind to BP180. These results demonstrate the ability of a BP180 conjugate to mediate selective degradation of anti-BP180 antibodies in a cellular assay.

### Example 7. PR3 Conjugate-Mediated Internalization and Degradation of a Pathogenic Antibody

In this example, a conjugate based on PR3 fused to an HSA polypeptide (a conjugate targeted to PR3 antibodies) was evaluated for the ability to mediate pathogenic antibody uptake and degradation in cells. The PR3 protein construct contains a point mutation to inhibit protease activity and mutations to stabilize the monomeric form of the protein and is further described herein.

Conjugate-mediated cellular uptake of a pathogenic antibody was tested in the Hep G2 human hepatocellular carcinoma cell line. 200,000 wild type or ASGPR ½ knock out Hep G2 cells were seeded into 24-well plates and incubated at 37° C. for 48 hr. Conjugates or unconjugated proteins were mixed in equimolar ratios with an antibody labeled with Alexa Fluor 647 and serial diluted in 5-fold steps from 300 nM to 2.4 nM before incubating at 22° C. After 30 minutes, media was removed from the cells and the conjugate or protein with antibody solutions were added to the cells and incubated for 2 hr at 37° C. Cells were then washed with phosphate buffered saline, lifted with TrypLE and analyzed for fluorescence signal by flow cytometry.

FIGS. 13A-13B show PR3 conjugate-mediated cellular uptake of the pathogenic antibody 4A5 (Hattar K, et al, J Immunol, 2002). Activity was tested with three groups and compared to signal from cells alone in wild type (WT) Hep G2 cells or Hep G2 ASGPR½ knockout (KO) cells. Results are presented as mean fluorescence intensity (MFI) vs. protein/conjugate-antibody complex concentration. The stabilized PR3 (SEQ ID NO: 12) was produced as a fusion with a HSA carrier polypeptide with a poly-his tag (SEQ ID NO: 2) to provide a PR3-HSA fusion protein (SEQ ID NO: 19) and conjugated to 2346. The treatment groups were as follows: group 1) antibody alone, group 2) PR3-HSA with antibody, group 3) PR3-HSA-2346 with antibody. A concentration-dependent increase in fluorescence signal was observed for 4A5 in the presence of the PR3 conjugate, while lower fluorescence signal was observed with antibody alone or in the presence of unconjugated PR3-HSA. Significantly lower fluorescence signal was observed with any treatment condition in HepG2 ASGPR½ KO cells. These results demonstrate that antibody uptake is dependent on the ASGPR internalizing receptor and the PR3-HSA-ASGPR ligand conjugate.

Conjugate-mediated antibody degradation was monitored indirectly using a labeled goat anti-human IgG F(ab′)₂ fragment (Jackson ImmunoResearch) probe. The F(ab′)₂ fragment was dual labeled with TAMRA and the QSY7 quencher as described (Wen Y, et al. American Assoc Pharm Sci J, 2020). Upon degradation of the probe, an increase in TAMRA fluorescence is observed. 200,000 wild type Hep G2 cells were seeded into 24-well plates and incubated at 37° C. for 48 hr. Conjugates or unconjugated proteins were mixed in equimolar ratios with unlabeled antibodies and the degradation probe giving a final concentration of 50 nM of each component in complete growth media. After 30 minutes, media was removed from the cells and the treatment solutions were added to the cells. Fluorescence was monitored every hour for 98 hrs using an Incucyte S3 imaging system (Sartorius). Each condition was tested in duplicate, and the integrated fluorescence intensity from 3 images in each well were used to generate an average integrated intensity at each time point.

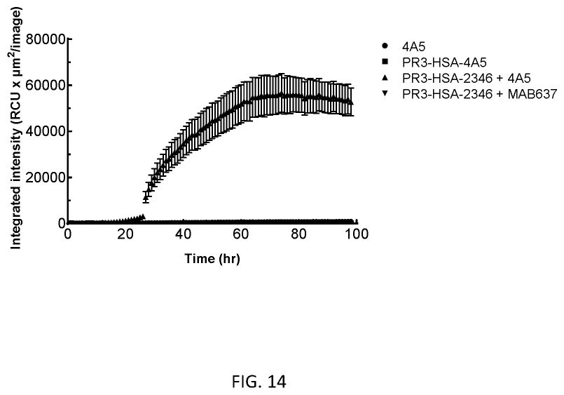

FIG. 14 illustrates the ability of a PR3 conjugate to induce degradation of the pathogenic antibody 4A5 in Hep G2 cells. Four treatment groups were tested: group 1) 4A5, group 2) PR3-HSA with 4A5, group 3) PR3-HSA-2346 with 4A5 and group 4) PR3-HSA-2346 with MAB637 (anti-AChR antibody). All groups also contained the anti-human IgG F(ab′)₂ probe labeled with TAMRA and QSY7 that binds to the pathogenic antibodies. In the group containing PR3-HSA-2346 and 4A5, an increase in fluorescence occurs beginning at about 20 hrs after treatment and peaks at about 60 hrs after treatment. In contrast, groups containing either antibody alone or antibody with PR3-HSA show minimal increase in signal over the course of the experiment. Antibody degradation is selective, as minimal signal increase is observed in the group containing PR3-HSA-2346 and MAB637, which does not bind to PR3. These results demonstrate the ability of a PR3 conjugate to mediate selective degradation of anti-PR3 antibodies in a cellular assay.

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All publications, patents, patent applications, and other documents cited in this application are hereby incorporated by reference in their entireties for all purposes to the same extent as if each individual publication, patent, patent application, or other document were individually indicated to be incorporated by reference for all purposes.

What is claimed is:

1. An extracellular target binding conjugate of formula (I): 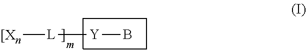
or a pharmaceutically acceptable salt thereof, wherein:
X is a moiety that binds to a lysosomal targeting molecule;
n is 1 to 50;
L is a linker;
m is the average number of (Xₙ-L) moieties conjugated to Y—B, wherein m is in the range from 1 to 20:
Y is an optional carrier polypeptide connected to B; and
B is a polypeptide that specifically binds an extracellular target molecule.

2. The conjugate of claim 1, wherein B specifically binds a target antibody.

3. The conjugate of claim 2, wherein the target antibody is an autoantibody.

4. The conjugate of claim 2, wherein the target antibody is a neutralizing antibody or an anti-drug antibody.

5. The conjugate of claim 2, wherein B comprises an antigen of the target antibody, or fragment thereof.

6. The conjugate of claim 2, wherein B comprises a protein domain.

7. The conjugate of claim 1, wherein Y comprises a protein domain.

8. The conjugate of claim 1, wherein Y is fused directly to B.

9. The conjugate of claim 1, wherein Y is fused indirectly to B via a spacer domain.

10. The conjugate of claim 1, wherein Y—B is a chimeric protein.

11. The conjugate of claim 1, wherein Y is selected from human serum albumin (HSA), HSA domain, albumin binding domain, Fc (monomer), Fc (dimer), and fragments thereof (e.g., synthetic peptides).

12. The conjugate of claim 10, wherein Y—B is selected from an Fc-VHH antigen fusion, an Fc-antigen fusion, and an HSA-antigen fusion.

13. The conjugate of claim 12, wherein Y—B is an HSA-antigen fusion.

14. The conjugate of claim 1, wherein Y is covalently linked to B via a linker.

15. The conjugate of claim 14, wherein the linker comprises a non-peptidic linking moiety.

16. The conjugate of any one of claims 14 to 15, wherein Y is selected from human serum albumin (HSA), HSA domain, albumin binding domain, Fc (monomer), Fc (dimer), and fragments thereof (e.g., synthetic peptides).

17. The conjugate of any preceding claim, wherein the conjugate is of formula (I′): 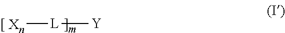
or a prodrug thereof, or a or a pharmaceutically acceptable salt thereof,
wherein:
n is 1 to 500;
m is 1 to 20;
X is the ligand moiety; and
Y is the target-binding moiety.

18. The conjugate of any preceding claim, wherein the lysosomal targeting molecule is a cell surface receptor that provides for internalization of the conjugate.

19. The conjugate of any preceding claim, wherein the lysosomal targeting molecule is selected from asialoglycoprotein receptor (ASGPR), cation independent mannose-6-phosphate receptor (CI-M6PR also referred to herein as M6PR), folate receptor, LDLR, CD63, sortilin, IFITM3, molecules in the endosome/lysosome pathway, LIMP-1, and LIMP-2.

20. The conjugate of any preceding claim, wherein X is a moiety that binds ASGPR or M6PR.

21. The conjugate of any preceding claim, wherein X is a moiety that binds ASGPR.

22. The conjugate of claim 21, wherein the conjugate is of formula (I′): 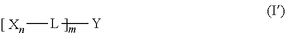
or a prodrug thereof, or a pharmaceutically acceptable salt thereof,
wherein:
n is 1 to 500:
m is 1 to 20;
L is a linker; and
X is an asialoglycoprotein receptor (ASGPR) binding moiety of formula (II): 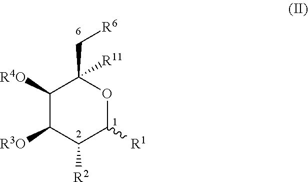
wherein:
R¹ is selected from Z¹—*, —H, —OH, optionally substituted (C₁-C₆)alkyl, —OCH₃, —OCH₂CH═CH, optionally substituted —S—(C₁-C₆)alkyl, optionally substituted aryl, optionally substituted heteroaryl, optionally substituted —S-aryl, and optionally substituted —S-heteroaryl;
R² is selected from —Z¹—*, —NHCOCH₃, —NHCOCF₃, —NHCOCH₂CF₃, —OH, —NHR, and optionally substituted triazole;
R⁶ is selected from —Z¹—*, —OH, —OR, optionally substituted (C₁-C₆)alkyl, —OC(O)R, —C(O)NHR, —NR^{xx}R^{yy}, optionally substituted aryl, optionally substituted heteroaryl, —NHCOR, and —NRCOR;
each R is independently optionally substituted (C₁-C₆)alkyl, optionally substituted aryl, or optionally substituted heteroaryl;
R^{xx} and R^{yy} are independently H, optionally substituted (C₁-C₆)alkyl, or R^{xx} and R^{yy} can cyclize to form an optionally substituted heterocyclyl;
wherein one of R¹, R², and R⁶ is —Z¹—*, and “*” represents a point of connection of Z¹ to the linker (L);
R³ and R⁴ are each independently H, or a promoiety, or R³ and R⁴ are cyclically linked to form a promoiety;
R¹¹ is H, or a bridging moiety that connects the 5-position carbon to the 1-position carbon of the ring;
Z¹ is a linking moiety selected from —Z¹¹—, —Z¹¹-A¹-, -A²-, —NR²¹CO—, —CONR²¹—, —NR²¹SO₂—, —SO₂NR²¹—, —NR²¹C(═O)NR²¹—, and —NR²¹C(═S)NR²¹—;
—Z¹¹— is —O—, —S—, —N(R²¹)—, or —C(R²²)₂:
-A¹- and -A²- are optionally substituted heterocyclylene, optionally substituted arylene, or optionally substituted heteroarylene;
each R²² is independently selected from H, optionally substituted (C₁-C₆)alkyl, —COR, and optionally substituted heteroaryl; and
each R²² is independently selected from H, halogen, and optionally substituted (C₁-C₆)alkyl.

23. The conjugate of claim 34, wherein -L-Y comprises: 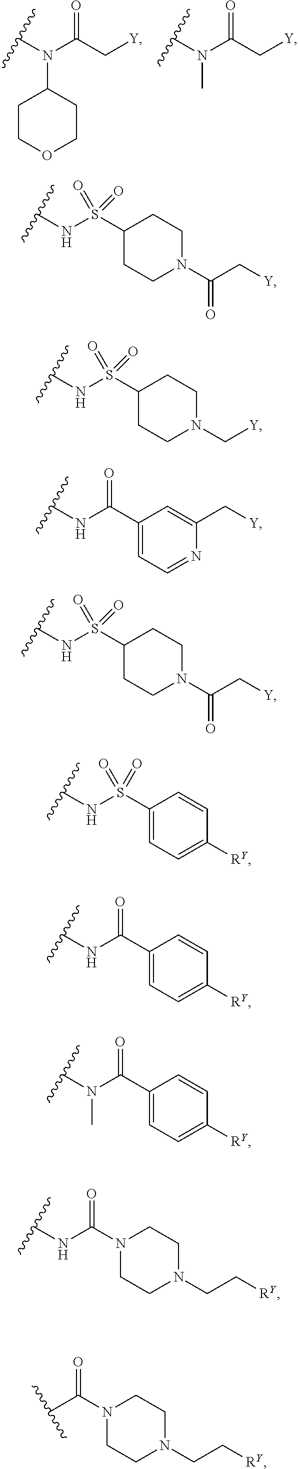 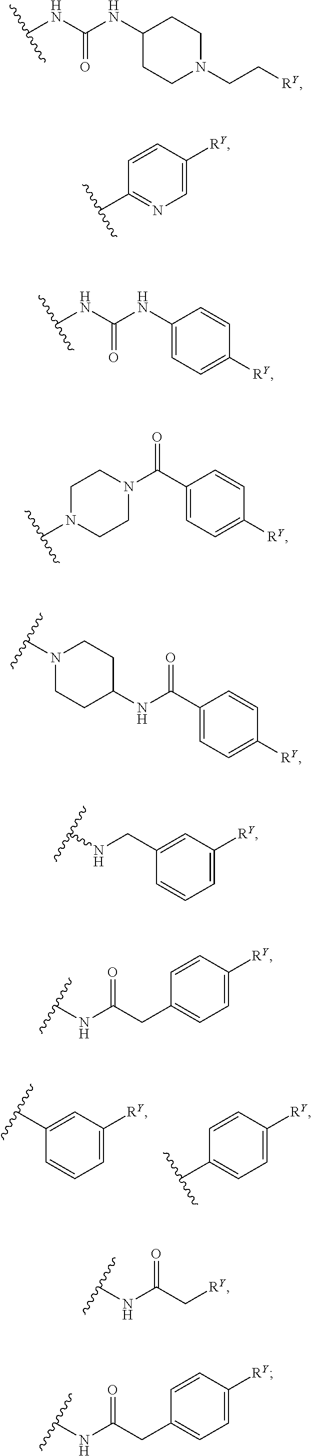
wherein R^{Y} is

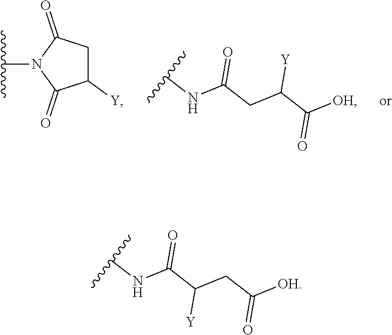

24. The conjugate of claim 22 or 23, wherein X is represented by formula (a-II):

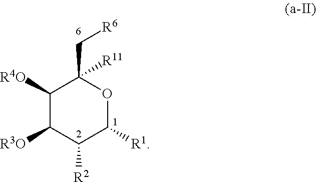

25. The conjugate of any one of claims 22-24, wherein R¹ is —Z¹—*, —H, or (C₁-C₆)alkyl.

26. The conjugate of any one of claims 22-24, wherein R² is —Z¹—* or —NHCOCH₃.

27. The conjugate of any one of claims 22-26, wherein R³ and R⁴ are each —H.

28. The conjugate of claim 20 wherein X is a moiety that binds to M6PR.

29. The conjugate of claim 28, wherein X is of formula (IV: 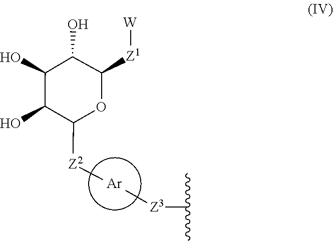
wherein:
W is a non-hydrolyzable hydrophilic head group;
Z¹ is selected from optionally substituted (C₁-C₃)alkylene and optionally substituted ethenylene;
Z² is selected from S, NR²¹ and C(R²²)₂, wherein each R²¹ is independently selected from H, and optionally substituted (C₁-C₆)alkyl, and each R²² is independently selected from H, halogen and optionally substituted (C₁-C₆)alkyl:
each A is independently an optionally substituted aryl or heteroaryl linking moiety; and
each Z³ is independently a linking moiety.

30. The conjugate of claim 29, wherein Z² is S.

31. The conjugate of claim 29 or 30, wherein W is phosphonate, thiophosphonate, carboxylic or malonic acid, or a salt thereof.

32. The conjugate of any one of claims 28-31, wherein X is: 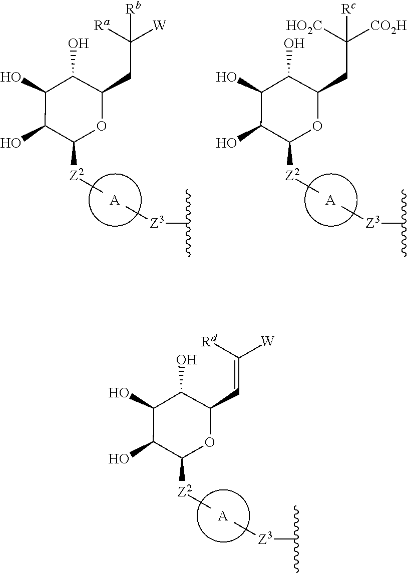
wherein R^{a}, R^{b}, R^{c} and R^{d} are independently H or F.

33. The conjugate of any one of claims 28-31, wherein X is: 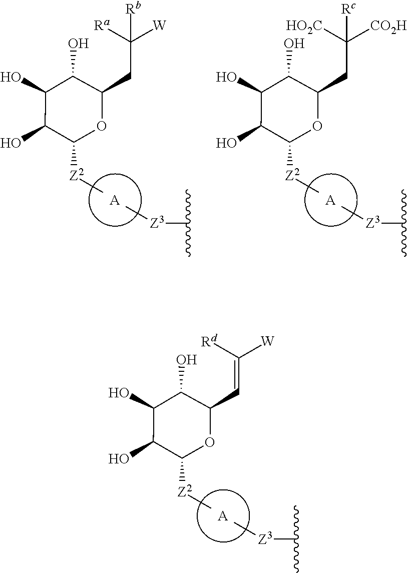
wherein R^{a}, R^{b}, R^{c} and R^{d} are independently H or F.

34. The conjugate of any one of claims 29-33, wherein A is optionally substituted aryl or optionally substituted heteroaryl, preferably A is independently selected from optionally substituted phenyl, optionally substituted pyridyl, optionally substituted biphenyl, optionally substituted naphthalene, optionally substituted triazole and optionally substituted phenylene-triazole.

35. The conjugate of any preceding claim, wherein L comprises of 10 to 60 consecutive branched or linear chain atoms.

36. The conjugate of any preceding claim, wherein L is of formula (IIb′): 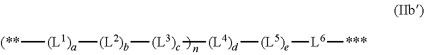
wherein:
n is 1, 2, or 3;
each L¹ to L⁶ is independently a linking moiety which together provide a linear or branched linker between Z¹ and Y;
a, b, c, d, and e are each independently 1, 2, 3, 4, or 5;
** represents the point of attachment to L¹ of X via Z¹; and
*** represents the point of attachment to Y.

37. The conjugate of claim 36, wherein each L¹ to L⁵ independently comprises one or more linking moieties independently selected from −C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50:
L⁶ is a linking group comprising one or more linking moieties independently selected from —C₁₋₂₀-alkylene-, —NR¹⁶C(O)—C₁₋₆-alkylene-, —C(O)NR¹⁶—C₁₋₆-alkylene-, —NR¹⁶—C₁₋₆-alkylene-, —NR¹⁶C(O)NR¹⁶—C₁₋₆-alkylene-, —NR¹⁶C(S)NR¹⁶—C₁₋₆-alkylene-, —C₁₋₆-alkylene-NR¹⁶C(O)—, —C₁₋₆-alkylene-C(O)NR¹⁶—, —C₁₋₆-alkylene-NR¹⁶—, —C₁₋₆-alkylene-NR¹⁶C(O)NR¹—, —C₁₋₆-alkylene-NR¹⁶C(S)NR¹⁶—, —O(CH₂), —, —(OCH₂CH₂)—, —NR¹⁶C(O)—, —C(O)NR¹⁶—, —NHS(O)₂—, —S(O)₂NH—, —C(O)—, —S(O)₂—, —O—, —S—, monocyclic heteroaryl, monocyclic aryl, monocyclic heterocycle, amino acid residue, or —NR¹⁶—; and
each R¹⁶ is independently —H, optionally substituted (C₁-C₆)alkyl, optionally substituted aryl, optionally substituted monocyclic heteroaryl or monocyclic heteroaryl.

38. The conjugate of claim 36, wherein each L¹ to L⁵ is independently selected from —C₁₋₂₀-alkylene-, —NHC(O)—C₁₋₆-alkylene-, —C(O)NH—C₁₋₆-alkylene-, —NH—C₁₋₆-alkylene-, —NHC(O)NH—C₁₋₆-alkylene-, —NHC(S)NH—C₁₋₆-alkylene-, —C₁₋₆-alkylene-NHC(O)—, —C₁₋₆-alkylene-C(O)NH—, —C₁₋₆-alkylene-NH—, —C₁₋₆-alkylene-NHC(O)NH—, —C₁₋₆-alkylene-NHC(S)NH—, —O(CH₂)ₚ—, —(OCH₂CH₂)ₚ—, —NHC(O)—, —C(O)NH—, —NHS(O)₂—, —S(O)₂NH—, —C(O)—, —S(O)₂—, —O—, —S—, monocyclic heteroaryl, monocyclic aryl, monocyclic heterocycle, monocyclic carbocycle, amino acid residue, —NH—, and —NMe-; wherein each L¹ to L⁵ is independently optionally substituted with one to five halo;
each p is independently 1 to 50; and
L⁶ is 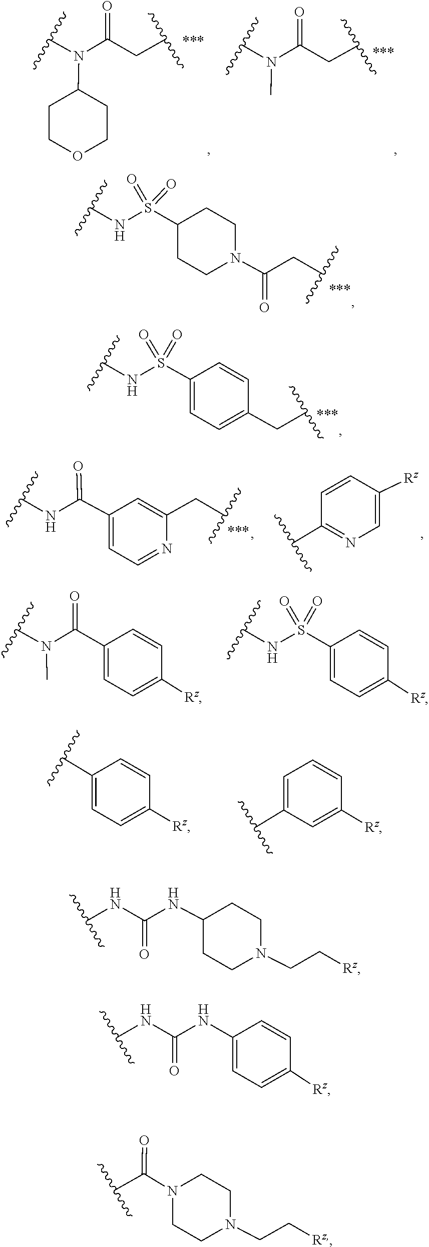 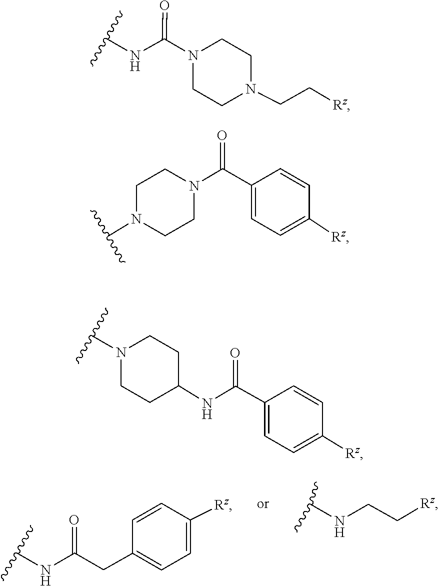
wherein R^{z} is

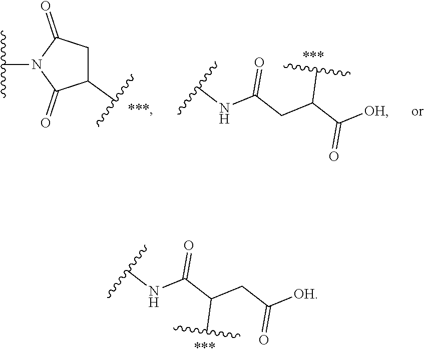 [SMILES: *C1CC(=O)N(*)C1=O ; *NC(=O)CC(*)C(=O)O ; *NC(=O)C(*)CC(=O)O]

39. A method of reducing levels of an extracellular target molecule in a biological system, the method comprising:
contacting the biological system with an effective amount of a conjugate according to any one of claims 1 to 38, wherein the compound specifically binds the extracellular target molecule and specifically binds a lysosomal targeting molecule of cells in the biological system to facilitate cellular uptake and degradation of the extracellular target molecule.
